(19) United States
(12) Patent Application Publication
(10) Pub. No.: US 2025/0041345 A1
(43) Pub. Date: Feb. 6, 2025

(54) MULTICISTRONIC CHIMERIC PROTEIN EXPRESSION SYSTEMS

(71) Applicant: Senti Biosciences, Inc., South San Francisco, CA (US)

(72) Inventors: Brian Scott Garrison, Saratoga, CA (US); Michelle Elizabeth Hung, South San Francisco, CA (US); Nicholas Frankel, San Francisco, CA (US); Han Deng, Millbrae, CA (US); Gozde Yucel, Fremont, CA (US)

(21) Appl. No.: 18/922,220

(22) Filed: Oct. 21, 2024

Related U.S. Application Data
(63) Continuation of application No. PCT/US2023/065860, filed on Apr. 17, 2023.
(60) Provisional application No. 63/440,668, filed on Jan. 23, 2023.
(60) Provisional application No. 63/370,219, filed on Aug. 2, 2022.
(60) Provisional application No. 63/333,483, filed on Apr. 21, 2022.

(51) Int. Cl. A61K 35/17; A61K 39/00; A61P 35/00; C07K 14/54; C07K 14/705; C07K 14/725; C07K 16/28; C07K 16/30

(52) U.S. Cl. CPC A61K 35/17; A61K 39/4613; A61K 39/4631; A61K 39/464403; A61K 39/464411; A61K 39/46447; A61P 35/00; C07K 14/5443; C07K 14/7051; C07K 14/70517; C07K 14/70532; C07K 16/2803; C07K 16/2863; C07K 16/3092; A61K 2239/13; A61K 2239/17; A61K 2239/21; C07K 2317/31; C07K 2317/35; C07K 2317/53; C07K 2317/565; C07K 2317/622; C07K 2319/02; C07K 2319/03; C07K 2319/50

(57) ABSTRACT

Described herein are multicistronic expression systems that encode chimeric proteins, specifically membrane-cleavable chimeric systems and chimeric antigen receptors. Also described herein are nucleic acids, cells, and methods directed to the same.

### CROSS REFERENCE TO RELATED APPLICATIONS

This application is a continuation of International Application No. PCT/US2023/065860, filed Apr. 17, 2023, which claims the benefit of each of U.S. Provisional Application Nos. 63/333,483 filed on Apr. 21, 2022; 63/370,219 filed on Aug. 2, 2022; and 63/440,668 filed Jan. 23, 2023, each of which is hereby incorporated in its entirety by reference.

### SEQUENCE LISTING

The instant application contains a Sequence Listing which has been submitted electronically in XML format and is hereby incorporated by reference in its entirety. Said XML copy, created on 27 Jul. 2023, is named STB-041WO and is 660,642 bytes in size.

### BACKGROUND

Cell-based therapy platforms provide promising avenues for treating a variety of diseases. One such promising platform is CAR-T based therapies in the treatment of cancer. Given their promise, improvements in cell-based therapies are needed. An active area of exploration is engineering cell-based therapies to produce and/or secrete effector molecules such as cytokines, a process referred to as armoring, that enhance the cell-based therapy. For example, unarmored CAR-T therapies have poor efficacy in solid tumors and armoring can impact the entire cancer immunity cycle and boost the activity of CAR-T. However, uncontrolled or unregulated armoring strategies can have negative impacts on treatment, such as off-target effects and toxicity in subjects. Thus, additional methods of controlling and regulating the armoring of cell-based therapies, such as regulating production and/or secretion of payload effector molecules, are required.

### SUMMARY

Provided herein, in some embodiments, is a cell-based therapy platform involving regulated armoring of the cell-based therapy, including chimeric antigen receptor (CAR)-based therapies (e.g. NK CARs), such as regulated secretion of payload effector molecules. Also provided herein, in some embodiments, is a combinatorial cell-based immunotherapy involving regulated armoring for the targeted treatment of cancer, such as ovarian cancer, breast cancer, colon cancer, lung cancer, and pancreatic cancer.

The therapy provided herein, however, can limit systemic toxicity of armoring. For example, the immunotherapy provided herein can be tumor-specific and effective while limiting systemic toxicity and/or other off-target effects due to armoring. These therapies deliver proteins of interest, such as immunomodulatory effector molecules, in particular IL-15, in a regulated manner, including regulation of secretion kinetics, cell state specificity, and cell or tissue specificity. The design of the delivery vehicle is optimized to improve overall function in cell-based therapies, such as cancer therapy, including, but not limited to, optimization of the membrane-cleavage sites, promoters, linkers, signal peptides, delivery methods, combination, regulation, and order of the immunomodulatory effector molecules.

The design of the delivery vehicle is also optimized for expression of multiple components for CAR-based therapies in a multicistronic system, including chimeric antigen receptors (both activating aCARs and inhibitory iCARs) and membrane-cleavable chimeric protein.

Provided for herein is a multicistronic expression system comprising an engineered nucleic acid comprising: (A) an exogenous polynucleotide encoding a membrane-cleavable chimeric protein, oriented from N-terminal to C-terminal, having the formula: S—C-MT or MT-C—S, wherein S comprises a secretable effector molecule, wherein the secretable effector molecule comprises IL-15, C comprises a protease cleavage site, and MT comprises a cell membrane tethering domain, wherein S—C-MT or MT-C—S is configured to be expressed as a single polypeptide; (B) an exogenous polynucleotide encoding an inhibitory chimeric antigen receptor (iCAR) comprising: (i) an antigen-binding domain specific for endomucin (EMCN); (ii) one or more intracellular inhibitory domains that inhibit an immune response, and (iii) one or more polypeptides selected from the group consisting of: a signal peptide, a transmembrane domain, a hinge domain, a spacer region, one or more peptide linkers, and combinations thereof; and (C) an exogenous polynucleotide encoding a bivalent activating chimeric antigen receptor (aCAR) comprising: (i) an antigen-binding domain specific for FLT3; (ii) an antigen-binding domain specific for CD33; (iii) one or more intracellular signaling domains that stimulate an immune response, and (iv) one or more polypeptides selected from the group consisting of: a signal peptide, a transmembrane domain, a hinge domain, a spacer region, one or more peptide linkers, and combinations thereof.

In some aspects, the exogenous polynucleotides encoding each of the membrane-cleavable chimeric protein, the iCAR, and the bivalent aCAR are linked together by a polynucleotide linker encoding a 2A ribosome skipping element. In some aspects, the 2A ribosome skipping element is selected from the group consisting of: a T2A ribosome skipping element, a E2A ribosome skipping element, a P2A ribosome skipping element, a F2A ribosome skipping element, ribosome skipping element fusions thereof, and combinations thereof. In some aspects, the ribosome skipping element fusion comprises an E2A/T2A ribosome skipping element. In some aspects, the E2A/T2A ribosome skipping element comprises the amino acid sequence QCTNYALLKLAGDVESNPGPGSGEGRGSLLTCGDVEENPGP (SEQ ID NO: 221). In some aspects, the E2A/T2A ribosome skipping element is encoded by a polynucleotide sequence comprising the sequence CAGTGTACCAACTACGCCCTGCTGAAACTGGCCGGCGACGTGGAATCTAATCCTGG ACCTGGATCTGGCGAGGGACGCGGGAGTCTACTGACGTGTGGAGACGTGGAGGAA AACCCTGGACCT (SEQ ID NO: 222) or the sequence CAGTGCACAAATTATGCACTGCTGAAGCTCGCCGGGGATGTCGAGAGTAACCCAGG ACCTGGAAGCGGAGAAGGTCGTGGTAGTCTACTAACGTGTGGTGATGTAGAAGAAA ATCCTGGACCT (SEQ ID NO: 223).

In some aspects, the membrane-cleavable chimeric protein, the iCAR, and the bivalent aCAR are encoded in order from 5′ to 3′: (i) membrane-cleavable chimeric protein; (ii) bivalent aCAR; and (iii) iCAR. In some aspects, the membrane-cleavable chimeric protein, the iCAR, and the bivalent aCAR are encoded in order from 5′ to 3′: (i) membrane-cleavable chimeric protein; (ii) iCAR; and (iii) bivalent aCAR.

In some aspects, the secretable effector molecule comprises a signal peptide or a signal-anchor sequence. In some aspects, the signal peptide comprises a native signal peptide native to the secretable effector molecule. In some aspects, the signal peptide comprises a non-native signal peptide or the signal-anchor sequence comprises a non-native signal-anchor sequence non-native to the secretable effector molecule. In some aspects, the non-native signal peptide or the non-native signal-anchor sequence is selected from the group consisting of: IgE, IL-12, IL-2, optimized IL-2, trypsiongen-2, Gaussia luciferase, CD5, human IgKVII, murine IgKVII, VSV-G, prolactin, serum albumin preprotein, azurocidin preprotein, osteonectin, CD33, IL-6, IL-8, CCL2, TIMP2, VEGFB, osteoprotegerin, serpin E1, GROalpha, CXCL12, IL-21, CD8, NKG2D, TNFR2, GMCSF, and GM-CSFRa. In some aspects, the non-native signal peptide comprises an IgE signal peptide. In some aspects, the IgE signal peptide comprises the amino acid sequence MDWTWILFLVAAATRVHS (SEQ ID NO: 228). In some aspects, the IgE signal peptide is encoded by a polynucleotide sequence comprising the sequence

(SEQ ID NO: 229)
ATGGACTGGACTTGGATACTCTTTCTGGTCGCTGCCGCCACACGG
GTGCACTCT.

In some aspects, the IL-15 comprises the amino acid sequence NWVNVISDLKKIEDLIQSMHIDATLYTESDVHPSCKVTAMKCFLLELQVISLESGDASIH DTVENLIILANNSLSSNGNVTESGCKECEELEEKNIKEFLQSFVHIVQMFINTS (SEQ ID NO: 224). In some aspects, the IL-15 is encoded by a polynucleotide sequence comprising the sequence

(SEQ ID NO: 225)
AATTGGGTCAACGTGATCAGCGACCTGAAGAAGATCGAGGACCTG
ATCCAGAGCATGCACATCGACGCCACACTGTACACCGAGTCCGAT
GTGCACCCTAGCTGCAAAGTGACCGCCATGAAGTGCTTTCTGCTG
GAACTGCAAGTGATCAGCCTGGAAAGCGGCGACGCCAGCATCCAC
GATACCGTGGAAAATCTGATCATCCTGGCCAACAACAGCCTGTCC
AGCAACGGCAATGTGACCGAGAGCGGCTGCAAAGAGTGCGAGGAA
CTGGAAGAGAAGAACATCAAAGAGTTTCTGCAGAGCTTCGTCCAC
ATCGTGCAGATGTTCATCAACACCTCA.

In some aspects, the protease cleavage site further comprises the N-terminal peptide linker, optionally selected from the group consisting of: SGGGGSGGGGSG (SEQ ID NO: 230); GGGSGGGGSGGGSLQ (SEQ ID NO: 231); GGS (SEQ ID NO: 232); GGSGGS (SEQ ID NO: 233); GGSGGSGGS (SEQ ID NO: 234); GGSGGSGGSGGS (SEQ ID NO: 235); GGSGGSGGSGGSGGS (SEQ ID NO: 236); GGGS (SEQ ID NO: 237); GGGSGGGS (SEQ ID NO: 238); GGGSGGGSGGGS (SEQ ID NO: 239); GGGSGGGSGGGSGGGS (SEQ ID NO: 240); GGGSGGGSGGGSGGGSGGGS (SEQ ID NO: 241); GGGGS (SEQ ID NO: 242); GGGGSGGGGS (SEQ ID NO: 243); GGGGSGGGGSGGGGS (SEQ ID NO: 244); GGGGSGGGGSGGGGSGGGGS (SEQ ID NO: 245); GGGGSGGGGSGGGGSGGGGSGGGGS (SEQ ID NO: 246); GSTSGSGKPGSGEGSTKG (SEQ ID NO: 247); and EAAAKEAAAKEAAAKEAAAK (SEQ ID NO: 248). In some aspects, the protease cleavage site further comprises the N-terminal peptide linker SGGGGSGGGGSG (SEQ ID NO: 230).

In some aspects, the protease cleavage site further comprises a C-terminal peptide linker, optionally selected from the group consisting of: SGGGGSGGGGSG (SEQ ID NO: 230); GGGSGGGGSGGGSLQ (SEQ ID NO: 231); GGS (SEQ ID NO: 232); GGSGGS (SEQ ID NO: 233); GGSGGSGGS (SEQ ID NO: 234); GGSGGSGGSGGS (SEQ ID NO: 235); GGSGGSGGSGGSGGS (SEQ ID NO: 236); GGGS (SEQ ID NO: 237); GGGSGGGS (SEQ ID NO: 238); GGGSGGGSGGGS (SEQ ID NO: 239); GGGSGGGSGGGSGGGS (SEQ ID NO: 240); GGGSGGGSGGGSGGGSGGGS (SEQ ID NO: 241); GGGGS (SEQ ID NO: 242); GGGGSGGGGS (SEQ ID NO: 243); GGGGSGGGGSGGGGS (SEQ ID NO: 244); GGGGSGGGGSGGGGSGGGGS (SEQ ID NO: 245); GGGGSGGGGSGGGGSGGGGSGGGGS (SEQ ID NO: 246); GSTSGSGKPGSGEGSTKG (SEQ ID NO: 247); and EAAAKEAAAKEAAAKEAAAK (SEQ ID NO: 248). In some aspects, the protease cleavage site further comprises the C-terminal linker GGGSGGGGSGGGSLQ (SEQ ID NO: 231).

In some aspects, the protease cleavage site comprises a Tumor Necrosis Factor-α Converting Enzyme (TACE)-specific cleavage site. In some aspects, the TACE-specific cleavage site comprises the amino acid sequence VTPEPIFSLI (SEQ ID NO: 191). In some aspects, the TACE-specific cleavage site comprises the amino acid sequence SGGGGSGGGGSGVTPEPIFSLIGGGSGGGGSGGGSLQ (SEQ ID NO: 250). In some aspects, the TACE-specific cleavage site is encoded by a polynucleotide sequence comprising the sequence

(SEQ ID NO: 251)
TCAGGCGGCGGTGGTAGTGGAGGCGGAGGCTCAGGCGTGACCCCT
GAGCCTATCTTCAGCCTGATCGGCGGAGGTTCCGGAGGTGGCGGT
TCCGGCGGAGGATCTCTTCAA.

In some aspects, the cell membrane tethering domain comprises a transmembrane domain selected from the group consisting of: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, LIR1, B7-1, and BTLA. In some aspects, the cell membrane tethering domain comprises a B7-1 transmembrane domain. In some aspects, B7-1 transmembrane domain comprises the amino acid sequence LLPSWAITLISVNGIFVICCLTYCFAPRCRERRRNERLRRESVRPV (SEQ ID NO: 204). In some aspects, B7-1 transmembrane domain is encoded by a polynucleotide sequence comprising the sequence

(SEQ ID NO: 252)
TTGCTGCCTAGCTGGGCCATCACACTGATCTCCGTGAACGGCATC
TTCGTGATCTGCTGCCTGACCTACTGCTTCGCCCCTAGATGCAGA
GAGCGGAGAAGAAACGAGCGGCTGAGAAGAGAAAGCGTGCGGCCT
GTG.

In some aspects, the membrane-cleavable chimeric protein, oriented from N-terminal to C-terminal, has the formula S—C-MT. In some aspects, the membrane-cleavable chimeric protein comprises the amino acid sequence MDWTWILFLVAAATRVHSNWVNVISDLKKIEDLIQSMHIDATLYTESDVHPSCKVTAM KCFLLELQVISLESGDASIHDTVENLIILANNSLSSNGNVTESGCKECEELEEKNIKEFLQS FVHIVQMFINTSSGGGGSGGGGSGVTPEPIFSLIGGGSGGGGSGGGSLQLLPSWAITLISV NGIFVICCLTYCFAPRCRERRRNERLRRESVRPV (SEQ ID NO: 226). In some aspects, the membrane-cleavable chimeric protein is encoded by a polynucleotide sequence comprising the sequence

(SEQ ID NO: 227)
ATGGACTGGACTTGGATACTCTTTCTGGTCGCTGCCGCCACACGG
GTGCACTCTAATTGGGTCAACGTGATCAGCGACCTGAAGAAGATC
GAGGACCTGATCCAGAGCATGCACATCGACGCCACACTGTACACC
GAGTCCGATGTGCACCCTAGCTGCAAAGTGACCGCCATGAAGTGC
TTTCTGCTGGAACTGCAAGTGATCAGCCTGGAAAGCGGCGACGCC
AGCATCCACGATACCGTGGAAAATCTGATCATCCTGGCCAACAAC
AGCCTGTCCAGCAACGGCAATGTGACCGAGAGCGGCTGCAAAGAG
TGCGAGGAACTGGAAGAGAAGAACATCAAAGAGTTTCTGCAGAGC
TTCGTCCACATCGTGCAGATGTTCATCAACACCTCATCAGGCGGC
GGTGGTAGTGGAGGCGGAGGCTCAGGCGTGACCCCTGAGCCTATC
TTCAGCCTGATCGGCGGAGGTTCCGGAGGTGGCGGTTCCGGCGGA
GGATCTCTTCAATTGCTGCCTAGCTGGGCCATCACACTGATCTCC
GTGAACGGCATCTTCGTGATCTGCTGCCTGACCTACTGCTTCGCC
CCTAGATGCAGAGAGCGGAGAAGAAACGAGCGGCTGAGAAGAGAA
AGCGTGCGGCCTGTG.

In some aspects, the antigen-binding domain specific for EMCN comprises a heavy chain variable (VH) region and a light chain variable (VL) region, wherein: the EMCN-VH comprises: a heavy chain complementarity determining region 1 (CDR-H1) having the amino acid sequence of RYDMH (SEQ ID NO: 291), a heavy chain complementarity determining region 2 (CDR-H2) having the amino acid sequence of VIWGNGNTHYHSALKS (SEQ ID NO: 296), and a heavy chain complementarity determining region 3 (CDR-H3) having the amino acid sequence of RIKD (SEQ ID NO: 298), and the EMCN-VL comprises: a light chain complementarity determining region 1 (CDR-L1) having the amino acid sequence of KSSQSLVASDENTYLN (SEQ ID NO: 299), a light chain complementarity determining region 2 (CDR-L2) having the amino acid sequence of QVSKLDS (SEQ ID NO: 300), and a light chain complementarity determining region 3 (CDR-L3) having the amino acid sequence of LQGIHLPWT (SEQ ID NO: 301), and wherein the amino acid sequences of the CDR-H1, the CDR-H2, the CDR-H3, the CDR-L1, the CDR-L2, and the CDR-L3 of the reference antibody are defined based on the Kabat numbering scheme. In some aspects, the EMCN-VH comprises the amino acid sequence EVOLVESGGGLVQPGGSLRLSCAASGFTFSRYDMHWVRQAPGKGLEWVSVIWGNGNT HYHSALKSRFTISRDNSKNTLYLQMNSLRAEDTAVYYCTLRIKDWGQGTMVTVSS (SEQ ID NO: 302); and the EMCN-VL comprises the amino acid sequence DVVMTQSPLSLPVTLGQPASISCKSSQSLVASDENTYLNWFQQRPGQSPRRLIYQVSKL DSGVPDRFSGSGSGTDFTLKISRVEAEDVGVYYCLQGIHLPWTFGQGTKLEIK (SEQ ID NO: 310).

In some aspects, the EMCN-VH and the EMCN-VL are separated by a peptide linker. In some aspects, the antigen-binding domain specific for EMCN comprises the structure VH-L-VL or VL-L-VH, wherein L is the peptide linker. In some aspects, the peptide linker comprises the amino acid sequence SGGGGSGGGGSG (SEQ ID NO: 230); GGGSGGGGSGGGSLQ (SEQ ID NO: 231); GGS (SEQ ID NO: 232); GGSGGS (SEQ ID NO: 233); GGSGGSGGS (SEQ ID NO: 234); GGSGGSGGSGGS (SEQ ID NO: 235); GGSGGSGGSGGSGGS (SEQ ID NO: 236); GGGS (SEQ ID NO: 237); GGGSGGGS (SEQ ID NO: 238); GGGSGGGSGGGS (SEQ ID NO: 239); GGGSGGGSGGGSGGGS (SEQ ID NO: 240); GGGSGGGSGGGSGGGSGGGS (SEQ ID NO: 241); GGGGS (SEQ ID NO: 242); GGGGSGGGGS (SEQ ID NO: 243); GGGGSGGGGSGGGGS (SEQ ID NO: 244); GGGGSGGGGSGGGGSGGGGS (SEQ ID NO: 245); GGGGSGGGGSGGGGSGGGGSGGGGS (SEQ ID NO: 246); GSTSGSGKPGSGEGSTKG (SEQ ID NO: 247); or EAAAKEAAAKEAAAKEAAAK (SEQ ID NO: 248). In some aspects, the peptide linker comprises the amino acid sequence GGGGSGGGGSGGGGS (SEQ ID NO: 244). In some aspects, the peptide linker is encoded by a polynucleotide sequence comprising the sequence

(SEQ ID NO: 253)
GGAGGCGGAGGATCTGGTGGCGGAGGAAGTGGCGGAGGCGGTTCT.

In some aspects, the antigen-binding domain specific for EMCN comprises the structure VH-L-VL and comprises the amino acid sequence EVOLVESGGGLVQPGGSLRLSCAASGFTFSRYDMHWVRQAPGKGLEWVSVIWGNGNT HYHSALKSRFTISRDNSKNTLYLQMNSLRAEDTAVYYCTLRIKDWGQGTMVTVSSGGG GSGGGGSGGGGSDVVMTQSPLSLPVTLGQPASISCKSSQSLVASDENTYLNWFQQRPG QSPRRLIYQVSKLDSGVPDRFSGSGSGTDFTLKISRVEAEDVGVYYCLQGIHLPWTFGQ GTKLEIK (SEQ ID NO: 311). In some aspects, the antigen-binding domain specific for EMCN is encoded by a polynucleotide sequence comprising the sequence

(SEQ ID NO: 312)
GAGGTGCAGCTGGTTGAATCTGGCGGAGGACTGGTTCAGCCTGGC
GGATCTCTGAGACTGTCTTGTGCCGCCAGCGGCTTCACCTTCAGC
AGATACGATATGCACTGGGTCCGACAGGCCCCTGGCAAAGGACTT
GAATGGGTGTCCGTGATCTGGGGCAACGGCAACACACACTACCAC
AGCGCCCTGAAGTCCCGGTTCACCATCTCCAGAGACAACAGCAAG
AACACCCTGTACCTGCAGATGAACAGCCTGAGAGCCGAGGACACC
GCCGTGTACTACTGCACCCTGAGAATCAAGGATTGGGGCCAGGGC
ACCATGGTCACCGTTTCTTCTGGAGGCGGAGGATCTGGTGGCGGA
GGAAGTGGCGGAGGCGGTTCTGACGTGGTCATGACACAGAGCCCT
CTGAGCCTGCCTGTGACACTGGGACAGCCTGCCAGCATCAGCTGC
AAGTCTAGCCAGTCTCTGGTGGCCAGCGACGAGAACACCTACCTG
AACTGGTTCCAGCAGAGGCCCGGACAGTCTCCTAGACGGCTGATC
TACCAGGTGTCCAAGCTGGATAGCGGCGTGCCCGATAGATTTTCT
GGCAGCGGCTCTGGCACCGACTTCACCCTGAAGATCAGCAGAGTG
GAAGCCGAGGACGTGGGCGTGTACTACTGTCTGCAAGGCATCCAT
CTGCCTTGGACCTTTGGCCAGGGCACAAAGCTGGAAATCAAG.

In some aspects, the intracellular inhibitory domain comprises a LIR1 intracellular inhibitory domain. In some aspects, the LIR1 intracellular inhibitory domain comprises the amino acid sequence LRHRRQGKHWTSTQRKADFQHPAGAVGPEPTDRGLQWRSSPAADAQEENLYAAVKH TQPEDGVEMDTRSPHDEDPQAVTYAEVKHSRPRREMASPPSPLSGEFLDTKDRQAEED RQMDTEAAASEAPQDVTYAQLHSLTLRREATEPPPSQEGPSPAVPSIYATLAIH (SEQ ID NO: 285). In some aspects, the LIR1 intracellular inhibitory domain is encoded by a polynucleotide sequence comprising the sequence

(SEQ ID NO: 286)
CTGCGGCACAGAAGGCAGGGCAAGCACTGGACAAGCACCCAGAGA
AAGGCCGACTTTCAGCATCCTGCTGGCGCCGTTGGACCTGAGCCT
ACAGATAGAGGACTGCAGTGGCGGTCTAGCCCTGCCGCTGATGCC
CAAGAGGAAAATCTTTACGCCGCCGTGAAGCACACCCAGCCTGAG
GATGGCGTGGAAATGGACACCAGATCTCCCCACGATGAGGACCCT
CAGGCCGTGACATACGCAGAAGTGAAGCACTCCAGACCTCGGAGA
GAGATGGCAAGCCCTCCATCTCCTCTGAGCGGCGAGTTCCTGGAC
ACCAAAGACAGACAGGCCGAAGAGGACAGACAGATGGATACCGAA
GCCGCCGCTTCTGAAGCCCCACAGGATGTGACATATGCCCAGCTG
CATAGCCTGACACTGCGGAGAGAAGCCACAGAGCCTCCACCTTCT
CAAGAAGGCCCATCTCCTGCCGTGCCTTCCATCTATGCCACTCTG
GCCATTCAC.

In some aspects, the signal peptide is present in the iCAR. In some aspects, the signal peptide of the iCAR comprises a native signal peptide native or a non-native signal peptide, optionally wherein the non-native signal peptide or the non-native signal-anchor sequence is selected from the group consisting of: IgE, IL-12, IL-2, optimized IL-2, trypsiongen-2, Gaussia luciferase, CD5, human IgKVII, murine IgKVII, VSV-G, prolactin, serum albumin preprotein, azurocidin preprotein, osteonectin, CD33, IL-6, IL-8, CCL2, TIMP2, VEGFB, osteoprotegerin, serpin E1, GROalpha, CXCL12, IL-21, CD8, NKG2D, TNFR2, and GMCSF. In some aspects, the signal peptide of the iCAR comprises a CD8 signal peptide. In some aspects, the CD8 signal peptide comprises the amino acid sequence MALPVTALLLPLALLLHAARP (SEQ ID NO: 137). In some aspects, the CD8 signal peptide is encoded by a polynucleotide sequence comprising the sequence

(SEQ ID NO: 416)
ATGGCTCTGCCCGTGACAGCTTTGCTGCTGCCTTTGGCACTGCTG
CTGCATGCTGCTAGACCA.

In some aspects, the hinge domain is present in the iCAR comprises. In some aspects, the hinge domain of the iCAR is selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof. In some aspects, the hinge domain of the iCAR comprises a LIR1 hinge. In some aspects, the LIR1 hinge comprises the amino acid sequence HPSDPLELVVSGPSGGPSSPTTGPTSTSGPEDQPLTPTGSDPQSGLGRHLGV (SEQ ID NO: 417). In some aspects, the LIR1 hinge comprises is encoded by a polynucleotide sequence comprising the sequence CACCCATCCGATCCTCTCGAGCTGGTGGTTTCTGGACCTTCTGGCGGCCCTAGCAGC CCTACAACAGGACCTACAAGCACAAGCGGCCCTGAGGACCAACCTCTGACACCAAC AGGCAGCGATCCTCAGTCTGGACTGGGGAGACATCTGGGCGTT (SEQ ID NO: 418). In some aspects, the hinge domain of the iCAR comprises a CD8 hinge. In some aspects, the CD8 hinge comprises the amino acid sequence TTTPAPRPPTPAPTIALQPLSLRPEACRPAAGGAVHTRGLDFACD (SEQ ID NO: 271). In some aspects, the CD8 hinge comprises is encoded by a polynucleotide sequence comprising the sequence

(SEQ ID NO: 283)
ACAACAACACCCGCACCTCGGCCTCCAACTCCAGCTCCAACAATT
GCACTGCAACCCCTGAGTCTGAGGCCCGAGGCCTGTAGGCCAGCA
GCTGGCGGAGCTGTTCACACTAGAGGCCTGGACTTTGCCTGTGAC.

In some aspects, the transmembrane domain of the iCAR is present. In some aspects, the transmembrane domain of the iCAR comprises a transmembrane domain selected from the group consisting of: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, LIR1, and BTLA. In some aspects, the transmembrane domain of the iCAR comprises a LIR1 transmembrane domain. In some aspects, the LIR1 transmembrane domain comprises the amino acid sequence VIGILVAVILLLLLLLLLFLI (SEQ ID NO: 259). In some aspects, the LIR1 transmembrane domain is encoded by a polynucleotide sequence comprising the sequence

(SEQ ID NO: 260)
GTGATCGGCATTCTGGTCGCCGTGATCCTGCTCCTGTTGCTCCTG
CTGCTTCTGTTCCTGATC.

In some aspects, the iCAR comprises the amino acid sequence

(SEQ ID NO: 430)
MALPVTALLLPLALLLHAARPEVOLVESGGGLVQPGGSLRLSCAA
SGFTFSRYDMHWVRQAPGKGLEWVSVIWGNGNTHYHSALKSRFTI
SRDNSKNTLYLQMNSLRAEDTAVYYCTLRIKDWGQGTMVTVSSGG
GGSGGGGSGGGGSDVVMTQSPLSLPVTLGQPASISCKSSQSLVAS
DENTYLNWFQQRPGQSPRRLIYQVSKLDSGVPDRFSGSGSGTDFT
LKISRVEAEDVGVYYCLQGIHLPWTFGQGTKLEIKHPSDPLELVV
SGPSGGPSSPTTGPTSTSGPEDQPLTPTGSDPQSGLGRHLGVVIG
ILVAVILLLLLLLLLFLILRHRRQGKHWTSTQRKADFQHPAGAVG
PEPTDRGLQWRSSPAADAQEENLYAAVKHTQPEDGVEMDTRSPHD
EDPQAVTYAEVKHSRPRREMASPPSPLSGEFLDTKDRQAEEDRQM
DTEAAASEAPQDVTYAQLHSLTLRREATEPPPSQEGPSPAVPSIY
ATLAIH;
or
(SEQ ID NO: 419)
MALPVTALLLPLALLLHAARPEVOLVESGGGLVQPGGSLRLSCAA
SGFTFSRYDMHWVRQAPGKGLEWVSVIWGNGNTHYHSALKSRFTI
SRDNSKNTLYLQMNSLRAEDTAVYYCTLRIKDWGQGTMVTVSSGG
GGSGGGGSGGGGSDVVMTQSPLSLPVTLGQPASISCKSSQSLVAS
DENTYLNWFQQRPGQSPRRLIYQVSKLDSGVPDRFSGSGSGTDFT
LKISRVEAEDVGVYYCLQGIHLPWTFGQGTKLEIKngaaHPSDPL
ELVVSGPSGGPSSPTTGPTSTSGPEDQPLTPTGSDPQSGLGRHLG
VVIGILVAVILLLLLLLLLFLILRHRRQGKHWTSTQRKADFQHPA
GAVGPEPTDRGLQWRSSPAADAQEENLYAAVKHTQPEDGVEMDTR
SPHDEDPQAVTYAEVKHSRPRREMASPPSPLSGEFLDTKDRQAEE
DRQMDTEAAASEAPQDVTYAQLHSLTLRREATEPPPSQEGPSPAV
PSIYATLAIH.

In some aspects, the iCAR is encoded by a polynucleotide sequence comprising the sequence

(SEQ ID NO: 420)
ATGGCTCTGCCCGTGACAGCTTTGCTGCTGCCTTTGGCACTGCTG
CTGCATGCTGCTAGACCAGAGGTGCAGCTGGTTGAATCTGGCGGA
GGACTGGTTCAGCCTGGCGGATCTCTGAGACTGTCTTGTGCCGCC
AGCGGCTTCACCTTCAGCAGATACGATATGCACTGGGTCCGACAG
GCCCCTGGCAAAGGACTTGAATGGGTGTCCGTGATCTGGGGCAAC
GGCAACACACACTACCACAGCGCCCTGAAGTCCCGGTTCACCATC
TCCAGAGACAACAGCAAGAACACCCTGTACCTGCAGATGAACAGC
CTGAGAGCCGAGGACACCGCCGTGTACTACTGCACCCTGAGAATC
AAGGATTGGGGCCAGGGCACCATGGTCACCGTTTCTTCTGGAGGC
GGAGGATCTGGTGGCGGAGGAAGTGGCGGAGGCGGTTCTGACGTG
GTCATGACACAGAGCCCTCTGAGCCTGCCTGTGACACTGGGACAG
CCTGCCAGCATCAGCTGCAAGTCTAGCCAGTCTCTGGTGGCCAGC
GACGAGAACACCTACCTGAACTGGTTCCAGCAGAGGCCCGGACAG
TCTCCTAGACGGCTGATCTACCAGGTGTCCAAGCTGGATAGCGGC
GTGCCCGATAGATTTTCTGGCAGCGGCTCTGGCACCGACTTCACC
CTGAAGATCAGCAGAGTGGAAGCCGAGGACGTGGGCGTGTACTAC
TGTCTGCAAGGCATCCATCTGCCTTGGACCTTTGGCCAGGGCACA
AAGCTGGAAATCAAGAATGGCGCTGCACACCCATCCGATCCTCTC
GAGCTGGTGGTTTCTGGACCTTCTGGCGGCCCTAGCAGCCCTACA
ACAGGACCTACAAGCACAAGCGGCCCTGAGGACCAACCTCTGACA
CCAACAGGCAGCGATCCTCAGTCTGGACTGGGGAGACATCTGGGC
GTTGTGATCGGCATTCTGGTCGCCGTGATCCTGCTCCTGTTGCTC
CTGCTGCTTCTGTTCCTGATCCTGCGGCACAGAAGGCAGGGCAAG
CACTGGACAAGCACCCAGAGAAAGGCCGACTTTCAGCATCCTGCT
GGCGCCGTTGGACCTGAGCCTACAGATAGAGGACTGCAGTGGCGG
TCTAGCCCTGCCGCTGATGCCCAAGAGGAAAATCTTTACGCCGCC
GTGAAGCACACCCAGCCTGAGGATGGCGTGGAAATGGACACCAGA
TCTCCCCACGATGAGGACCCTCAGGCCGTGACATACGCAGAAGTG
AAGCACTCCAGACCTCGGAGAGAGATGGCAAGCCCTCCATCTCCT
CTGAGCGGCGAGTTCCTGGACACCAAAGACAGACAGGCCGAAGAG
GACAGACAGATGGATACCGAAGCCGCCGCTTCTGAAGCCCCACAG
GATGTGACATATGCCCAGCTGCATAGCCTGACACTGCGGAGAGAA
GCCACAGAGCCTCCACCTTCTCAAGAAGGCCCATCTCCTGCCGTG
CCTTCCATCTATGCCACTCTGGCCATTCAC.

In some aspects, the iCAR comprises the amino acid sequence

(SEQ ID NO: 431)
MALPVTALLLPLALLLHAARPEVOLVESGGGLVQPGGSLRLSCAA
SGFTFSRYDMHWVRQAPGKGLEWVSVIWGNGNTHYHSALKSRFTI
SRDNSKNTLYLQMNSLRAEDTAVYYCTLRIKDWGQGTMVTVSSGG
GGSGGGGSGGGGSDVVMTQSPLSLPVTLGQPASISCKSSQSLVAS
DENTYLNWFQQRPGQSPRRLIYQVSKLDSGVPDRFSGSGSGTDFT
LKISRVEAEDVGVYYCLQGIHLPWTFGQGTKLEIKTTTPAPRPPT
PAPTIALQPLSLRPEACRPAAGGAVHTRGLDFACDVIGILVAVI
LLLLLLLLLFLILRHRRQGKHWTSTQRKADFQHPAGAVGPEPTDR
GLQWRSSPAADAQEENLYAAVKHTQPEDGVEMDTRSPHDEDPQAV
TYAEVKHSRPRREMASPPSPLSGEFLDTKDRQAEEDRQMDTEAAA
SEAPQDVTYAQLHSLTLRREATEPPPSQEGPSPAVPSIYATLAIH;
or
(SEQ ID NO: 421)
MALPVTALLLPLALLLHAARPEVOLVESGGGLVQPGGSLRLSCAA
SGFTFSRYDMHWVRQAPGKGLEWVSVIWGNGNTHYHSALKSRFTI
SRDNSKNTLYLQMNSLRAEDTAVYYCTLRIKDWGQGTMVTVSSGG
GGSGGGGSGGGGSDVVMTQSPLSLPVTLGQPASISCKSSQSLVAS
DENTYLNWFQQRPGQSPRRLIYQVSKLDSGVPDRFSGSGSGTDFT
LKISRVEAEDVGVYYCLQGIHLPWTFGQGTKLEIKngaaTTTPAP
RPPTPAPTIALQPLSLRPEACRPAAGGAVHTRGLDFACDVIGILV
AVILLLLLLLLLFLILRHRRQGKHWTSTQRKADFQHPAGAVGPEP
TDRGLQWRSSPAADAQEENLYAAVKHTQPEDGVEMDTRSPHDEDP
QAVTYAEVKHSRPRREMASPPSPLSGEFLDTKDRQAEEDRQMDTE
AAASEAPQDVTYAQLHSLTLRREATEPPPSQEGPSPAVPSIYATL
AIH.

In some aspects, the iCAR is encoded by a polynucleotide sequence comprising the sequence

(SEQ ID NO: 422)
ATGGCTCTGCCCGTGACAGCTTTGCTGCTGCCTTTGGCACTGCTG
CTGCATGCTGCTAGACCAGAGGTGCAGCTGGTTGAATCTGGCGGA
GGACTGGTTCAGCCTGGCGGATCTCTGAGACTGTCTTGTGCCGCC
AGCGGCTTCACCTTCAGCAGATACGATATGCACTGGGTCCGACAG
GCCCCTGGCAAAGGACTTGAATGGGTGTCCGTGATCTGGGGCAAC
GGCAACACACACTACCACAGCGCCCTGAAGTCCCGGTTCACCATC
TCCAGAGACAACAGCAAGAACACCCTGTACCTGCAGATGAACAGC
CTGAGAGCCGAGGACACCGCCGTGTACTACTGCACCCTGAGAATC
AAGGATTGGGGCCAGGGCACCATGGTCACCGTTTCTTCTGGAGGC
GGAGGATCTGGTGGCGGAGGAAGTGGCGGAGGCGGTTCTGACGTG
GTCATGACACAGAGCCCTCTGAGCCTGCCTGTGACACTGGGACAG
CCTGCCAGCATCAGCTGCAAGTCTAGCCAGTCTCTGGTGGCCAGC
GACGAGAACACCTACCTGAACTGGTTCCAGCAGAGGCCCGGACAG
TCTCCTAGACGGCTGATCTACCAGGTGTCCAAGCTGGATAGCGGC
GTGCCCGATAGATTTTCTGGCAGCGGCTCTGGCACCGACTTCACC
CTGAAGATCAGCAGAGTGGAAGCCGAGGACGTGGGCGTGTACTAC
TGTCTGCAAGGCATCCATCTGCCTTGGACCTTTGGCCAGGGCACA
AAGCTGGAAATCAAGAATGGCGCTGCAACAACAACACCCGCACCT
CGGCCTCCAACTCCAGCTCCAACAATTGCACTGCAACCCCTGAGT
CTGAGGCCCGAGGCCTGTAGGCCAGCAGCTGGCGGAGCTGTTCAC
ACTAGAGGCCTGGACTTTGCCTGTGACGTGATCGGCATTCTGGTC
GCCGTGATCCTGCTCCTGTTGCTCCTGCTGCTTCTGTTCCTGATC
CTGCGGCACAGAAGGCAGGGCAAGCACTGGACAAGCACCCAGAGA
AAGGCCGACTTTCAGCATCCTGCTGGCGCCGTTGGACCTGAGCCT
ACAGATAGAGGACTGCAGTGGCGGTCTAGCCCTGCCGCTGATGCC
CAAGAGGAAAATCTTTACGCCGCCGTGAAGCACACCCAGCCTGAG
GATGGCGTGGAAATGGACACCAGATCTCCCCACGATGAGGACCCT
CAGGCCGTGACATACGCAGAAGTGAAGCACTCCAGACCTCGGAGA
GAGATGGCAAGCCCTCCATCTCCTCTGAGCGGCGAGTTCCTGGAC
ACCAAAGACAGACAGGCCGAAGAGGACAGACAGATGGATACCGAA
GCCGCCGCTTCTGAAGCCCCACAGGATGTGACATATGCCCAGCTG
CATAGCCTGACACTGCGGAGAGAAGCCACAGAGCCTCCACCTTCT
CAAGAAGGCCCATCTCCTGCCGTGCCTTCCATCTATGCCACTCTG
GCCATTCAC.

In some aspects, the antigen-binding domain specific for FLT3 comprises a heavy chain variable (VH) region and a light chain variable (VL) region, wherein: the FLT3-VH comprises: a heavy chain complementarity determining region 1 (CDR-H1) having the amino acid sequence of GGTFSSYAIS (SEQ ID NO: 360), a heavy chain complementarity determining region 2 (CDR-H2) having the amino acid sequence of GIIPIFGTANYAQKFQG (SEQ ID NO: 361), and a heavy chain complementarity determining region 3 (CDR-H3) having the amino acid sequence of FALFGFREQAFDI (SEQ ID NO: 362), and the FLT3-VL comprises: a light chain complementarity determining region 1 (CDR-L1) having the amino acid sequence of RASQSISSYLN (SEQ ID NO: 363), a light chain complementarity determining region 2 (CDR-L2) having the amino acid sequence of AASSLQS (SEQ ID NO: 364), and a light chain complementarity determining region 3 (CDR-L3) having the amino acid sequence of QQSYSTPFT (SEQ ID NO: 365), and wherein the amino acid sequences of the CDR-H1, the CDR-H2, the CDR-H3, the CDR-L1, the CDR-L2, and the CDR-L3 of the reference antibody are defined based on the Kabat numbering scheme. In some aspects, the FLT3-VH comprises the amino acid sequence EVQLVQSGAEVKKPGSSVKVSCKASGGTFSSYAISWVRQAPGQGLEWMGGIIPIFGTAN YAQKFQGRVTITADKSTSTAYMELSSLRSEDTAVYYCATFALFGFREQAFDIWGQGTTV TVSS (SEQ ID NO: 315); and the FLT3-VL comprises the amino acid sequence DIQMTQSPSSLSASVGDRVTITCRASQSISSYLNWYQQKPGKAPKLLIYAASSLQSGVPS RFSGSGSGTDFTLTISSLQPEDLATYYCQQSYSTPFTFGPGTKVDIK (SEQ ID NO: 316).

In some aspects, the antigen-binding domain specific for CD33 comprises a heavy chain variable (VH) region and a light chain variable (VL) region, wherein: the CD33-VH comprises: a heavy chain complementarity determining region 1 (CDR-H1) having the amino acid sequence of DYNMH (SEQ ID NO: 402), a heavy chain complementarity determining region 2 (CDR-H2) having the amino acid sequence of YIYPYNGGTGYNQKFKSKA (SEQ ID NO: 403), and a heavy chain complementarity determining region 3 (CDR-H3) having the amino acid sequence of GRPAMDYWGQ (SEQ ID NO: 404), and the CD33-VL comprises: a light chain complementarity determining region 1 (CDR-L1) having the amino acid sequence of RASESVDNYGISFMN (SEQ ID NO: 405), a light chain complementarity determining region 2 (CDR-L2) having the amino acid sequence of AASNQGS (SEQ ID NO: 406), and a light chain complementarity determining region 3 (CDR-L3) having the amino acid sequence of QQSKEVPWT (SEQ ID NO: 407), and wherein the amino acid sequences of the CDR-H1, the CDR-H2, the CDR-H3, the CDR-L1, the CDR-L2, and the CDR-L3 of the reference antibody are defined based on the Kabat numbering scheme. In some aspects: the CD33-VH comprises the amino acid sequence QVQLVQSGAEVKKPGSSVKVSCKASGYTFTDYNMHWVRQAPGQGLEWIGYIYPYNGG TGYNQKFKSKATITADESTNTAYMELSSLRSEDTAVYYCARGRPAMDYWGQGTLVTV SS (SEQ ID NO: 329); and the CD33-VL comprises the amino acid sequence DIQMTQSPSSLSASVGDRVTITCRASESVDNYGISFMNWFQQKPGKAPKLLIYAASNQG SGVPSRFSGSGSGTDFTLTISSLQPDDFATYYCQQSKEVPWTFGQGTKVEIK (SEQ ID NO: 330).

In some aspects, the FLT3-VH, the FLT3-VL, the CD33-VH, and the CD33-VL are separated by peptide linkers. In some aspects, the aCAR antigen binding domains comprises the structure (FLT3-VH)-L1-(CD33-VH)-L2-(CD33-VL)-L3-(FLT3-VL), wherein L1, L2, and L3 are a first, a second, and a third peptide linker, respectively. In some aspects, the L1, L2, and/or L3 are each independently selected from the group consisting of: SGGGGSGGGGSG (SEQ ID NO: 230); GGGSGGGGSGGGSLQ (SEQ ID NO: 231); GGS (SEQ ID NO: 232); GGSGGS (SEQ ID NO: 233); GGSGGSGGS (SEQ ID NO: 234); GGSGGSGGSGGS (SEQ ID NO: 235); GGSGGSGGSGGSGGS (SEQ ID NO: 236); GGGS (SEQ ID NO: 237); GGGSGGGS (SEQ ID NO: 238); GGGSGGGSGGGS (SEQ ID NO: 239); GGGSGGGSGGGSGGGS (SEQ ID NO: 240); GGGSGGGSGGGSGGGSGGGS (SEQ ID NO: 241); GGGGS (SEQ ID NO: 242); GGGGSGGGGS (SEQ ID NO: 243); GGGGSGGGGSGGGGS (SEQ ID NO: 244); GGGGSGGGGSGGGGSGGGGS (SEQ ID NO: 245); GGGGSGGGGSGGGGSGGGGSGGGGS (SEQ ID NO: 246); GSTSGSGKPGSGEGSTKG (SEQ ID NO: 247); and EAAAKEAAAKEAAAKEAAAK (SEQ ID NO: 248). In some aspects, the L1 peptide linker is the amino acid sequence GGGGS (SEQ ID NO: 242) or GGGGSGGGGS (SEQ ID NO: 243). In some aspects, the L1 peptide linker GGGGS (SEQ ID NO: 242) is encoded by a polynucleotide sequence comprising the sequence GGCGGCGGTGGCTCT (SEQ ID NO: 254) or the L1 peptide linker GGGGSGGGGS (SEQ ID NO: 243) is encoded by a polynucleotide sequence comprising the sequence GGAGGCGGAGGATCTGGTGGTGGTGGATCT (SEQ ID NO: 256). In some aspects, the L2 peptide linker is the amino acid sequence GSTSGSGKPGSGEGSTKG (SEQ ID NO: 247). In some aspects, the L2 peptide linker is encoded by a polynucleotide sequence comprising the sequence GGCTCTACATCTGGCTCTGGCAAACCTGGAAGCGGCGAGGGATCTACCAAGGGC (SEQ ID NO: 249). In some aspects, the L2 peptide linker is the amino acid sequence GGGGGGGGS (SEQ ID NO: 243). In some aspects, the L2 peptide linker is encoded by a polynucleotide sequence comprising the sequence GGTGGCGGAGGAAGTGGCGGCGGAGGCTCT (SEQ ID NO: 257). In some aspects, the L3 peptide linker is the amino acid sequence GGGGS (SEQ ID NO: 242) or GGGGGGGGS (SEQ ID NO: 243). In some aspects, the L3 peptide linker GGGGS (SEQ ID NO: 242) is encoded by a polynucleotide sequence comprising the sequence GGTGGCGGCGGATCC (SEQ ID NO: 255) or the L3 peptide linker GGGGSGGGGS (SEQ ID NO: 243) is encoded by a polynucleotide sequence comprising the sequence GGCGGTGGCGGATCTGGCGGAGGTGGCAGT (SEQ ID NO: 258).

In some aspects, the aCAR intracellular signaling domains that stimulate an immune response is selected from the group consisting of: CD3-zeta, FcR gamma, FcR beta, CD3 gamma, CD3 delta, CD3 epsilon, CD5, CD22, CD79a, CD79b, CD278, FcεRI, DAP10, DAP12, CD66d, CD97, CD2, ICOS, CD27, CD154, CD8, OX40, 4-1BB, CD28, ZAP40, CD30, GITR, HVEM, DAP10, DAP12, MyD88, 2B4, CD40, PD-1, LFA-1, CD7, LIGHT, NKG2C, B7-H3, an MHC class I molecule, a TNF receptor protein, an Immunoglobulin-like protein, a cytokine receptor, an integrin, a SLAM protein, an activating NK cell receptor, BTLA, a Toll ligand receptor, CDS, ICAM-1, (CD11a/CD18), BAFFR, KIRDS2, SLAMF7, NKp80 (KLRF1), NKp44, NKp30, NKp46, CD19, CD4, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and combinations thereof. In some aspects, the aCAR intracellular signaling domains that stimulate an immune response comprise a CD28 co-stimulatory domain and a CD3ζ signaling domain. In some aspects, the CD28 co-stimulatory domain comprises the amino acid sequence RSKRSRLLHSDYMNMTPRRPGPTRKHYQPYAPPRDFAAYRS (SEQ ID NO: 287). In some aspects, the CD28 co-stimulatory domain is encoded by a polynucleotide sequence comprising the sequence AGAAGCAAGCGGAGCAGACTGCTGCACAGCGACTACATGAACATGACCCCTAGAC GGCCCGGACCTACCAGAAAGCACTACCAGCCTTACGCTCCTCCTAGAGATTTCGCC GCCTACCGGTCC (SEQ ID NO: 288). In some aspects, the CD3ζ signaling domain comprises the amino acid sequence RVKFSRSADAPAYKQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO: 289). In some aspects, the CD35 signaling domain is encoded by a polynucleotide sequence comprising the sequence

(SEQ ID NO: 290)
AGAGTGAAGTTCAGCAGGAGCGCAGACGCCCCCGCGTACAAGCAG
GGCCAGAACCAGCTCTATAACGAGCTCAATCTAGGACGAAGAGAG
GAGTACGATGTTTTGGACAAGAGACGTGGCCGGGACCCTGAGATG
GGGGGAAAGCCGAGAAGGAAGAACCCTCAGGAAGGCCTGTACAAT
GAACTGCAGAAAGATAAGATGGCGGAGGCCTACAGTGAGATTGGG
ATGAAAGGCGAGCGCCGGAGGGGCAAGGGGCACGATGGCCTTTAC
CAGGGTCTCAGTACAGCCACCAAGGACACCTACGACGCCCTTCAC
ATGCAGGCCCTGCCCCCTCGC.

In some aspects, the hinge domain of the aCAR is present. In some aspects, the hinge domain of the aCAR is selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof. In some aspects, the hinge domain of the aCAR comprises a CD8 hinge. In some aspects, the CD8 hinge comprises the amino acid sequence ALSNSIMYFSHFVPVFLPAKPTTTPAPRPPTPAPTIASQPLSLRPEACRPAAGGAVHTRGL DFACD (SEQ ID NO: 272). In some aspects, the CD8 hinge comprises is encoded by a polynucleotide sequence comprising the sequence

(SEQ ID NO: 284)
GCCCTGAGCAACAGCATCATGTACTTCAGCCACTTCGTGCCCGTG
TTTCTGCCCGCCAAGCCTACAACAACCCCTGCTCCTAGACCACCT
ACACCAGCTCCTACAATCGCCAGCCAGCCTCTGTCTCTGAGGCCC
GAAGCTTGTAGACCAGCTGCTGGCGGAGCCGTGCATACAAGAGGA
CTGGATTTTGCCTGCGAC.

In some aspects, the transmembrane domain of the aCAR is present. In some aspects, the transmembrane domain of the aCAR is selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof. In some aspects, the transmembrane domain of the aCAR comprises a CD8 hinge. In some aspects, the CD8 transmembrane comprises the amino acid sequence IYIWAPLAGTCGVLLLSLVITLYCNHR (SEQ ID NO: 206). In some aspects, the CD8 transmembrane comprises is encoded by a polynucleotide sequence comprising the sequence

(SEQ ID NO: 261)
ATCTATATCTGGGCCCCTCTGGCTGGCACATGCGGAGTTCTGCTG
CTCAGCCTGGTCATCACCCTGTACTGCAACCACAGA.

In some aspects, the aCAR signal peptide of the aCAR is present. In some aspects, the signal peptide of the aCAR is selected from the group consisting of: IgE, IL-12, IL-2, optimized IL-2, trypsiongen-2, Gaussia luciferase, CD5, human IgKVII, murine IgKVII, VSV-G, prolactin, serum albumin preprotein, azurocidin preprotein, osteonectin, CD33, IL-6, IL-8, CCL2, TIMP2, VEGFB, osteoprotegerin, serpin E1, GROalpha, CXCL12, IL-21, CD8, NKG2D, TNFR2, GMCSF, and GM-CSFRa. In some aspects, the signal peptide of the aCAR comprises a GM-CSFRa signal peptide. In some aspects, the GM-CSFRa signal peptide comprises the amino acid sequence MLLLVTSLLLCELPHPAFLLIP (SEQ ID NO: 423). In some aspects, the GM-CSFRa signal peptide is encoded by a polynucleotide sequence comprising the sequence

(SEQ ID NO: 424)
ATGCTGCTGCTGGTTACATCTCTGCTGCTGTGCGAGCTGCCCCAT
CCTGCCTTTCTGCTTATTCCT.

In some aspects, the aCAR comprises the amino acid sequence

(SEQ ID NO: 414)
MLLLVTSLLLCELPHPAFLLIPEVQLVQSGAEVKKPGSSVKVSCK
ASGGTFSSYAISWVRQAPGQGLEWMGGIIPIFGTANYAQKFQGRV
TITADKSTSTAYMELSSLRSEDTAVYYCATFALFGFREQAFDIWG
QGTTVTVSSGGGGSQVQLVQSGAEVKKPGSSVKVSCKASGYTFTD
YNMHWVRQAPGQGLEWIGYIYPYNGGTGYNQKFKSKATITADEST
NTAYMELSSLRSEDTAVYYCARGRPAMDYWGQGTLVTVSSGSTSG
SGKPGSGEGSTKGDIQMTQSPSSLSASVGDRVTITCRASESVDNY
GISFMNWFQQKPGKAPKLLIYAASNQGSGVPSRFSGSGSGTDFTL
TISSLQPDDFATYYCQQSKEVPWTFGQGTKVEIKGGGGSDIQMTQ
SPSSLSASVGDRVTITCRASQSISSYLNWYQQKPGKAPKLLIYAA
SSLQSGVPSRFSGSGSGTDFTLTISSLOPEDLATYYCQQSYSTPF
TFGPGTKVDIKALSNSIMYFSHFVPVFLPAKPTTTPAPRPPTPAP
TIASQPLSLRPEACRPAAGGAVHTRGLDFACDIYIWAPLAGTCGV
LLLSLVITLYCNHRRSKRSRLLHSDYMNMTPRRPGPTRKHYQPY
APPRDFAAYRSRVKFSRSADAPAYKQGQNQLYNELNLGRREEYDV
LDKRRGRDPEMGGKPRRKNPQEGLYNELQKDKMAEAYSEIGMKGE
RRRGKGHDGLYQGLSTATKDTYDALHMQALPPR.

In some aspects, the aCAR comprises the amino acid sequence

(SEQ ID NO: 415)
MLLLVTSLLLCELPHPAFLLIPEVQLVQSGAEVKKPGSSVKVSCK
ASGGTFSSYAISWVRQAPGQGLEWMGGIIPIFGTANYAQKFQGRV
TITADKSTSTAYMELSSLRSEDTAVYYCATFALFGFREQAFDIWG
QGTTVTVSSGGGGSGGGGSQVQLVQSGAEVKKPGSSVKVSCKASG
YTFTDYNMHWVRQAPGQGLEWIGYIYPYNGGTGYNQKFKSKATIT
ADESTNTAYMELSSLRSEDTAVYYCARGRPAMDYWGQGTLVTVSS
GGGGSGGGGSDIQMTQSPSSLSASVGDRVTITCRASESVDNYGIS
FMNWFQQKPGKAPKLLIYAASNQGSGVPSRFSGSGSGTDFTLTIS
SLQPDDFATYYCQQSKEVPWTFGQGTKVEIKGGGGSGGGGSDIQM
TQSPSSLSASVGDRVTITCRASQSISSYLNWYQQKPGKAPKLLIY
AASSLQSGVPSRFSGSGSGTDFTLTISSLQPEDLATYYCQQSYST
PFTFGPGTKVDIKALSNSIMYFSHFVPVFLPAKPTTTPAPRPPTP
APTIASQPLSLRPEACRPAAGGAVHTRGLDFACDIYIWAPLAGTC
GVLLLSLVITLYCNHRRSKRSRLLHSDYMNMTPRRPGPTRKHYQP
YAPPRDFAAYRSRVKFSRSADAPAYKQGQNQLYNELNLGRREEYD
VLDKRRGRDPEMGGKPRRKNPQEGLYNELQKDKMAEAYSEIGMKG
ERRRGKGHDGLYQGLSTATKDTYDALHMQALPPR.

Also provided herein is an expression vector comprising any of the multicistronic expression systems provided herein. In some aspects, the expression vector comprises the polynucleotide of SEQ ID NO: 425, SEQ ID NO: 426, SEQ ID NO: 427, SEQ ID NO: 428, SEQ ID NO: 433, SEQ ID NO: 432, or SEQ ID NO: 429.

Also provided herein is an isolated cell comprising any of the multicistronic expression systems provided herein or any of the expression vectors provided herein. In some aspects, the cell comprises an immune cell. In some aspects, the cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell. In some aspects, the cell is an NK cell.

Also provided herein is a polynucleotide comprising the nucleic acid sequence of SEQ ID NO: 425, SEQ ID NO: 426, SEQ ID NO: 427, SEQ ID NO: 428, SEQ ID NO: 433, SEQ ID NO: 432, or SEQ ID NO: 429.

Also provided herein is a polynucleotide comprising the nucleic acid sequence of SEQ ID NO: 425.

Also provided herein is a polynucleotide comprising the nucleic acid sequence of SEQ ID NO: 426.

Also provided herein is a polynucleotide comprising the nucleic acid sequence of SEQ ID NO: 427.

Also provided herein is a polynucleotide comprising the nucleic acid sequence of SEQ ID NO: 428.

Also provided herein is a polynucleotide comprising the nucleic acid sequence of SEQ ID NO: 429.

Also provided herein is a polynucleotide comprising the nucleic acid sequence of SEQ ID NO: 432.

Also provided herein is a polynucleotide comprising the nucleic acid sequence of SEQ ID NO: 433.

Also provided herein is a method of treating a subject in need thereof, the method comprising administering a therapeutically effective dose of any of the isolated cells provided herein or any of the polynucleotides provided herein.

Also provided herein is a method of treating a subject with cancer, the method comprising administering to the subject an immunotherapy comprising of any of the isolated cells provided herein or any of the polynucleotides provided herein.

A method of stimulating a cell-mediated immune response to a tumor cell in a subject, the method comprising administering to a subject having a tumor a therapeutically effective dose of any of the isolated cells of any of the isolated cells provided herein or any of the polynucleotides provided herein. In some aspects, the isolated cell is derived from the subject. In some aspects, the isolated cell is allogeneic with reference to the subject.

Also provided herein is a method of making an engineered cell, comprising transducing an isolated cell with any of the multicistronic expression systems provided herein, any of the expression vectors provided herein, or any of the polynucleotides provided herein.

### BRIEF DESCRIPTION OF THE DRAWINGS

FIG. 1 provides a schematic illustrating components and organization of the overall FLT3 OR CD33 NOT EMCN logic-gated CAR-NK cell system.

FIG. 2 provides a schematic illustrating components assessed in the optimization of the FLT3 OR CD33 NOT EMCN logic-gated CAR-NK cell system.

FIG. 3 shows the gating strategy for assessing iCAR and aCAR expression by flow cytometry.

FIG. 4 shows aCAR and iCAR expression at Day 15 for the indicated series of Retro Vec constructs.

FIG. 5 shows aCAR and iCAR expression at Day 15 for the indicated series of self-inactivated (SIN) γ-retroviral SINvec constructs.

FIG. 6 shows representative flow cytometry plots for membrane-bound IL-15 expression at Day 15 for the indicated series of Retro Vec constructs.

FIG. 7 shows representative flow cytometry plots for membrane-bound IL-15 expression at Day 15 for the indicated series of SINvec constructs.

FIG. 8 shows a first series of comparisons for aCAR, iCAR expression, and membrane-bound IL-15 for the indicated series of Retro Vec and SINvec constructs.

FIG. 9 shows a second series of comparisons for aCAR, iCAR expression, and membrane-bound IL-15 for the indicated series of Retro Vec and SINvec constructs.

FIG. 10 shows a third series of comparisons for aCAR, iCAR expression, and membrane-bound IL-15 for the indicated series of Retro Vec and SINvec constructs.

FIG. 11 shows a summary of aCAR and iCAR expression at Day 15 as assessed by MFI for the indicated series of Retro Vec and SINvec constructs.

FIG. 12 shows a general summary of expression considerations for the various components and organizations assessed.

FIG. 13 shows total cytotoxicity against target cell lines expressing FLT3/CD33 (AML cell line MV4-11) and target cell lines engineered to also express the NOT gate target antigen EMCN (MV4-11+EMCN) for constructs using a LIR1 ICD, both for constructs ordered in an aCAR to iCAR organization (top panel) and an iCAR to aCAR organization (bottom panel).

FIG. 14 shows normalized cytotoxicity against target cell lines expressing FLT3/CD33 (AML cell line MV4-11) and target cell lines engineered to also express the NOT gate target antigen EMCN (MV4-11+EMCN) for constructs using a LIR1 ICD.

FIG. 15 shows total cytotoxicity at Day 17 against target cell lines expressing FLT3/CD33 (AML cell line MV4-11) and target cell lines engineered to also express the NOT gate target antigen EMCN (MV4-11+EMCN) at the indicated effector:target ratios for constructs using a LIR1 ICD for constructs ordered in an aCAR to iCAR organization.

FIG. 16 shows normalized cytotoxicity at Day 10 against target cell lines expressing FLT3/CD33 (AML cell line MV4-11) and target cell lines engineered to also express the NOT gate target antigen EMCN (MV4-11+EMCN) at the indicated effector:target ratios for constructs using a LIR1 ICD, both for constructs ordered in an aCAR to iCAR organization (top panel) and an iCAR to aCAR organization (bottom panel).

FIG. 17 illustrates a schematic of the Membrane-Cleavable system described herein in which a desired payload is expressed as a chimeric protein in which a protease cleavage site is inserted between the payload and membrane-tethering domain. The left panel illustrates a schematic of the Membrane-Cleavable system using a transmembrane spanning architecture (e.g., the chimeric protein contains a transmembrane domain). The right panel illustrates a schematic of the Membrane-Cleavable system using a membrane-associated architecture (e.g., the chimeric protein contains a post-translational modification tag allowing association with a cell membrane).

FIG. 18 illustrates a representative Membrane-Cleavable system for the regulated secretion of IL-15 in which IL-15 is expressed as a chimeric protein having a cleavage site capable of cleavage by TACE inserted between the IL-15 payload and membrane-tethering domain. The left panel illustrates a Type I transmembrane architecture (e.g., S—C-MT) through use of Type I transmembrane domains, such as PDGFR-beta and CD8. The right panel a Type II transmembrane architecture (e.g., MT-C—S) through use of Type II transmembrane domains, such as NKG2D and TNFR2.

FIG. 19 shows components and their order of the indicated constructs encoding either bivalent FLT3 OR CD33 aCARs or monospecific aCARs.

FIG. 20 shows survival curves in MV4-11 murine tumor models for bivalent FLT3 OR CD33 CAR-NK cells and monospecific CAR-NK cells.

FIG. 21 shows representative tumor imaging in MV4-11 murine tumor models for bivalent FLT3 OR CD33 CAR-NK cells and monospecific CAR-NK cells.

FIG. 22 illustrates various tagged and tagless combinations of aCARs and iCARS.

FIG. 23 shows representative flow cytometry plots for a “no virus” control used for assessing tagged and tagless CARs.

FIG. 24 shows representative flow cytometry plots for tagless constructs SB07401 and SB07405 at Day 8.

FIG. 25 shows representative flow cytometry plots for iCAR tagged only construct SB06467 at Day 8.

FIG. 26 shows a summary of CD33/EMCN double positive cell percentage for the indicated constructs having either Loop 6 (top panel) or Loop 4 (bottom panel) linkers.

FIG. 27 shows a summary of expression for each of the CD33, FLT3, and EMCN CARs for the indicated constructs having Loop 4 linkers.

FIG. 28 shows a summary of expression for each of the CD33, FLT3, and EMCN CARs for the indicated constructs having Loop 6 linkers.

FIG. 29 shows total cytotoxicity against target cell lines expressing FLT3/CD33 (AML cell line MV4-11) and target cell lines engineered to also express the NOT gate target antigen EMCN (MV4-11+EMCN) at a 1:2 effector:target ratio for constructs using a LIR1 ICD.

FIG. 30 shows normalized cytotoxicity (normalized to no-virus control) against target cell lines expressing FLT3/CD33 (AML cell line MV4-11) and target cell lines engineered to also express the NOT gate target antigen EMCN (MV4-11+EMCN) at a 1:2 effector:target ratio for constructs using a LIR1 ICD.

FIG. 31 shows normalized cytotoxicity (normalized to no-virus control) against target cell lines expressing FLT3/CD33 (AML cell line MV4-11) and target cell lines engineered to also express the NOT gate target antigen EMCN (MV4-11+EMCN) at a 1:4 effector:target ratio for constructs using a LIR1 ICD.

FIG. 32 shows specific cytotoxicity of target cell lines expressing CD33 (SEM) and target cell lines also expressing the NOT gate target antigen (SEM+EMCN) at a 1:2 effector:target ratio. The figure provides SEM EMCN− in the left columns, and EMCN+ in the right columns, respectively.

FIG. 33A shows specific cytotoxicity of target cell lines expressing CD33 and target cell lines also expressing the NOT gate target antigen (SEM+EMCN) at a 1:2 effector:target ratio in a serial killing assay at 24 hours. FIG. 33B shows specific cytotoxicity of target cell lines expressing CD33 and target cell lines also expressing the NOT gate target antigen (SEM+EMCN) at a 1:2 effector:target ratio in a serial killing assay at 48 hours. FIG. 33C shows specific cytotoxicity of target cell lines expressing CD33 and target cell lines also expressing the NOT gate target antigen (SEM+EMCN) at a 1:2 effector:target ratio in a serial killing assay at 96 hours. EMCN+ right columns, respectively.

FIG. 34A shows a general schematic of the in vivo experiment to assess the NOT-logic gated CAR-NK cells. FIG. 34B shows the characterization the 50:50 mixture of SEM CD33+ and SEM CD33+/EMCN+ cells shown in FIG. 34A.

FIG. 35 shows the results of an in vivo experiment where a 1:1 mixture of SEM+/−EMCN was injected into mice and treated with NK cells transduced with the respective vector. The top row shows the BLI image at day 11, whereas the bottom panel shows the BLI image at 19 days after tumor injection.

FIG. 36 shows the results of FIG. 35, where the region of interest (ROI) is quantified at days 11 and 19.

FIG. 37A shows the proportion of healthy cells in mice on day 14 of the in vivo study where mice were injected with a 1:1 mixture of SEM+/−EMCN and treated with NK cells transduced with the respective constructs. The circled portion compares the percentage of EMCN+ SEM cells between OR/NOT- and OR-GATED CAR-NK cell treatments showing a significantly higher percentage of EMCN+ SEM cells in the animal treated with OR/NOT GATED CAR-NK cells, demonstrating NOT GATE protection of EMCN+ cells. FIG. 37B shows the proportion of EMCN+ SEM cells on day 21 of the study shown in FIG. 37A. FIG. 37C shows the proportion of EMCN+ SEM cells on day 27 of the study shown in FIG. 37A.

FIG. 38A shows the average BLI measurement of mice on the respective days of the in vivo study where mice were injected with MV4-11 cells and treated with NK cells transduced with the respective constructs. FIG. 38B shows the quantified BLI signal of the mice shown in FIG. 38A over the course of the in vivo study shown in FIG. 38A.

FIG. 39A shows the BLI measurement of mice on the respective days of the in vivo study where mice were injected with MV4-11 cells and treated with NK cells transduced with the respective constructs.

FIG. 39B shows a representative image on day 55 post tumor transplantation of mice treated with PBS, untransduced NK cells, or NK cells transduced with SB07412 or SB07418.

FIG. 39C shows a survival curve and the media survival (days) of the tested treated groups.

FIG. 39D shows a continuation of the study shown in FIG. 39C, where data has been collected until day 120.

FIG. 40 shows testing of NK cells expressing an aCAR and different iCAR intracellular domains in a cytotoxicity assay.

FIG. 41A shows a comparison of cell-mediated cytotoxicity against MOLM-13 using non-engineered or engineered NK cells.

FIG. 41B shows a comparison of cell-mediated cytotoxicity against MV4-11 using non-engineered or engineered NK cells.

FIG. 41C shows a comparison of cell-mediated cytotoxicity against SEM-CD33 using non-engineered or engineered NK cells.

FIG. 41D shows NK cytotoxicity of MOLM-13 cells at multiple E:T ratios.

FIG. 41E shows NK cytotoxicity of MV4-11 at multiple E:T ratios.

FIG. 41F shows NK cytotoxicity of engineered NK cells and non-engineered NK cells from multiple experiments plotted against CD33 CAR expression.

FIG. 42 shows a heat map to visualize fold expression analysis of cytokine production by NK cells following co-culture with MV4-11 or MOLM-13.

FIG. 43A shows NK-cell cytotolxicity of engineered and non engineered NK cells derived from donor A that are co-cultured with primary AML samples.

FIG. 43B shows NK-cell cytotolxicity of engineered and non engineered NK cells derived from donor B that are co-cultured with primary AML samples.

FIG. 44 shows a heat map analysis of NK activation markers as analyzed by flow cytometry.

FIG. 45A shows expression of surface associated IL15 in transduced NK cells derived from different donors.

FIG. 45B shows expression of secreted IL15 in transduced NK cells derived from different donors.

FIG. 45C shows expression of the CD33/FLT3 bivalent aCAR in transduced NK cells derived from different donors.

FIG. 45D shows expression of the EMCN iCAR in engineered NK cells derived from different donors.

FIG. 46A shows results from a cytotoxicity assay of engineered NK cells incubated with human stem cells expressing EMCN.

FIG. 46B shows results from a cytoxicity assay of engineered NK cells incubated with AML (Leukemia cells).

FIG. 46C shows results from a cytoxicity assay of engineerered NK cells incubated with human stem progenitor cells.

FIG. 46D shows results from a cytoxicity assay of engineered NK cells incubated with primary healthy MPPs. Shown are results for the whole healthy MPP population.

FIG. 46E shows results from a cytoxicity assay of engineered NK cells incubated with primary healthy MPPs. Shown are results for the healthy EMCN+ MPPs population.

FIG. 47 shows EMCN expression on various hematopoietic stem and progenitor cell (HSPC) subpopulations in CD34-enriched primary human bone marrow cells, including hematopoietic progenitor cells (HPC), lympho-myeloid primed progenitor cells (LMPPs), multipotent progenitor cells (MPPs), in addition to HSCs, across two representative donors.

FIG. 48 shows a flow cytometry gating strategy to count the number of live remaining EMCN+ HSCs after killing assay. From left to right and top to bottom, progressive gates on (1) cell size parameters, (2) single cells, (3) CD56-negative cells (i.e., not NK cells), (4) live CD34+ undifferentiated cells, (5) CD45RA-CD38-cells (i.e., HSCs and MPPs), (6) CD90+ cells (i.e., HSCs), and (7) EMCN+ HSCs. Gates for CD90 and EMCN were set using FMO controls.

### DETAILED DESCRIPTION

The present disclosure provides multicistronic expression systems. The multicistronic systems encode (i) a membrane-cleavable chimeric protein; (ii) a bivalent aCAR; and (iii) an iCAR. The multicistronic systems include FLT3 OR CD33 NOT EMCN logic-gated CARs and a membrane-cleavable chimeric protein encoding IL-15.

Provided are multicistronic expression systems that include an engineered nucleic acid encoding:
(A) an exogenous polynucleotide encoding a membrane-cleavable chimeric protein, oriented from N-terminal to C-terminal, having the formula: S—C-MT or MT-C—S wherein S includes a secretable effector molecule, wherein the secretable effector molecule includes IL-15, C includes a protease cleavage site, and MT includes a cell membrane tethering domain. S—C-MT or MT-C—S is configured to be expressed as a single polypeptide;
(B) an exogenous polynucleotide encoding an inhibitory chimeric antigen receptor (iCAR) including: (i) an antigen-binding domain specific for endomucin (EMCN); (ii) one or more intracellular inhibitory domains that inhibit an immune response, and (iii) one or more polypeptides selected from the group consisting of: a signal peptide, a transmembrane domain, a hinge domain, a spacer region, one or more peptide linkers, and combinations thereof; and
(C) an exogenous polynucleotide encoding a bivalent activating chimeric antigen receptor (aCAR) including: (i) an antigen-binding domain specific for FLT3; (ii) an antigen-binding domain specific for CD33; (iii) one or more intracellular signaling domains that stimulate an immune response, and (iv) one or more polypeptides selected from the group consisting of: a signal peptide, a transmembrane domain, a hinge domain, a spacer region, one or more peptide linkers, and combinations thereof.

The expression systems described herein are multicistronic, i.e., more than one separate polypeptide (e.g., multiple chimeric proteins) can be produced from a single mRNA transcript. Engineered nucleic acids can be multicistronic through the use of various linkers, e.g., a polynucleotide sequence encoding a first chimeric proteins can be linked to a nucleotide sequence encoding a second chimeric protein, such as in a first gene: linker: second gene 5′ to 3′ orientation. A linker can encode a 2A ribosome skipping element, such as T2A. Other 2A ribosome skipping elements include, but are not limited to, E2A, P2A, and F2A. 2A ribosome skipping elements allow production of separate polypeptides encoded by the first and second genes are produced during translation. 2A ribosome skipping elements can include fusion peptides of 2A ribosome skipping elements, including, but not limited to, an E2A/T2A ribosome skipping element. In certain embodiments, the E2A/T2A ribosome skipping element comprises the amino acid sequence of GSGQCTNYALLKLAGDVESNPGPGSGEGRGSLLTCGDVEENPGP (SEQ ID NO: 219). One exemplary nucleic acid encoding the E2A/T2A ribosome skipping element is GGTAGCGGCCAGTGTACCAACTACGCCCTGCTGAAACTGGCCGGCGACGTGGAATC TAATCCTGGACCTGGATCTGGCGAGGGACGCGGGAGTCTACTGACGTGTGGAGACG TGGAGGAAAACCCTGGACCT (SEQ ID NO: 220). In certain embodiments, a nucleic acid encoding the E2A/T2A ribosome skipping element includes a sequence that is at least 90%, at least 91%, at least 92%, at least 93%, at least 94%, at least 95%, at least 96%, at least 97%, at least 98%, or at least 99% identical to SEQ ID NO: 220. In some embodiments, an engineered nucleic acid disclosed herein comprises an E2A/T2A ribosome skipping element. In certain embodiments, the E2A/T2A ribosome skipping element comprises the amino acid sequence of QCTNYALLKLAGDVESNPGPGSGEGRGSLLTCGDVEENPGP (SEQ ID NO: 221). One exemplary nucleic acid encoding the E2A/T2A ribosome skipping element is CAGTGTACCAACTACGCCCTGCTGAAACTGGCCGGCGACGTGGAATCTAATCCTGG ACCTGGATCTGGCGAGGGACGCGGGAGTCTACTGACGTGTGGAGACGTGGAGGAA AACCCTGGACCT (SEQ ID NO: 222). Another exemplary nucleic acid encoding the E2A/T2A ribosome skipping element is CAGTGCACAAATTATGCACTGCTGAAGCTCGCCGGGGATGTCGAGAGTAACCCAGG ACCTGGAAGCGGAGAAGGTCGTGGTAGTCTACTAACGTGTGGTGATGTAGAAGAAA ATCCTGGACCT (SEQ ID NO: 223). In certain embodiments, a nucleic acid encoding the E2A/T2A ribosome skipping element includes a sequence that is at least 90%, at least 91%, at least 92%, at least 93%, at least 94%, at least 95%, at least 96%, at least 97%, at least 98%, or at least 99% identical to SEQ ID NO: 222 or SEQ ID NO: 223.

A linker can encode a cleavable linker polypeptide sequence, such as a Furin cleavage site or a TEV cleavage site, wherein following expression the cleavable linker polypeptide is cleaved such that separate polypeptides encoded by the first and second genes are produced. A cleavable linker can include a polypeptide sequence, such as such a flexible linker (e.g., a Gly-Ser-Gly sequence), that further promotes cleavage.

A linker can encode an Internal Ribosome Entry Site (IRES), such that separate polypeptides encoded by the first and second genes are produced during translation. A linker can encode a splice acceptor, such as a viral splice acceptor.

A linker can be a combination of linkers, such as a Furin-2A linker that can produce separate polypeptides through 2A ribosome skipping followed by further cleavage of the Furin site to allow for complete removal of 2A residues. In some embodiments, a combination of linkers can include a Furin sequence, a flexible linker, and 2A linker. Accordingly, in some embodiments, the linker is a Furin-Gly-Ser-Gly-2A fusion polypeptide. In some embodiments, a linker of the present disclosure is a Furin-Gly-Ser-Gly-T2A fusion polypeptide.

In general, a multicistronic system can use any number or combination of linkers, to express any number of genes or portions thereof (e.g., an engineered nucleic acid can encode a first, a second, and a third chimeric protein, each separated by linkers such that separate polypeptides encoded by the first, second, and third chimeric proteins are produced).

In general, the exogenous polynucleotides encoding each of the membrane-cleavable chimeric protein, the iCAR, and the bivalent aCAR can be encoded in any order. For example, the membrane-cleavable chimeric protein, the iCAR, and the bivalent aCAR can be encoded by the multicistronic system in order from 5′ to 3′: (i) membrane-cleavable chimeric protein; (ii) bivalent aCAR; and (iii) iCAR. In another example, the membrane-cleavable chimeric protein, the iCAR, and the bivalent aCAR can be encoded by the multicistronic system in order from 5′ to 3′: i) membrane-cleavable chimeric protein; (ii) iCAR; and (iii) bivalent aCAR.

### Membrane Cleavable Chimeric Proteins

The multicistronic systems herein encode membrane-cleavable chimeric proteins having the formula S—C-MT or MT-C—S, oriented from N-terminal to C-terminal and expressed as a single polypeptide. S refers to a secretable effector molecule. C refers to a protease cleavage site. MT refers to a cell membrane tethering domain. The membrane-cleavable chimeric protein is engineered such that secretion of the effector molecule can be regulated in a protease-dependent manner. Specifically, the membrane-cleavable chimeric protein is engineered such that secretion of the effector molecule can be regulated as part of a “Membrane-Cleavable” system, where incorporation of a protease cleavage site (“C”) and a cell membrane tethering domain (“MT”) allow for regulated secretion of an effector molecule in a protease-dependent manner. Without wishing to be bound by theory, the components of the Membrane-Cleavable system present in the membrane-cleavable chimeric protein generally regulate secretion through the below cellular processes:
MT: The cell membrane tethering domain contains a transmembrane domain (or a transmembrane-intracellular domain) that directs cellular-trafficking of the chimeric protein such that the protein is inserted into, or otherwise associated with, a cell membrane (“tethered”)
C: Following expression and localization of the chimeric protein into the cell membrane, the protease cleavage site directs cleavage of the chimeric protein such that the effector molecule is released (“secreted”) into the extracellular space. Generally, the protease cleavage site is protease-specific, including sites engineered to be protease-specific. The protease cleavage site can be selected or engineered to achieve optimal protein expression, cell-type specific cleavage, cell-state specific cleavage, and/or cleavage and release of the payload at desired kinetics (e.g., ratio of membrane-bound to secreted chimeric protein levels)

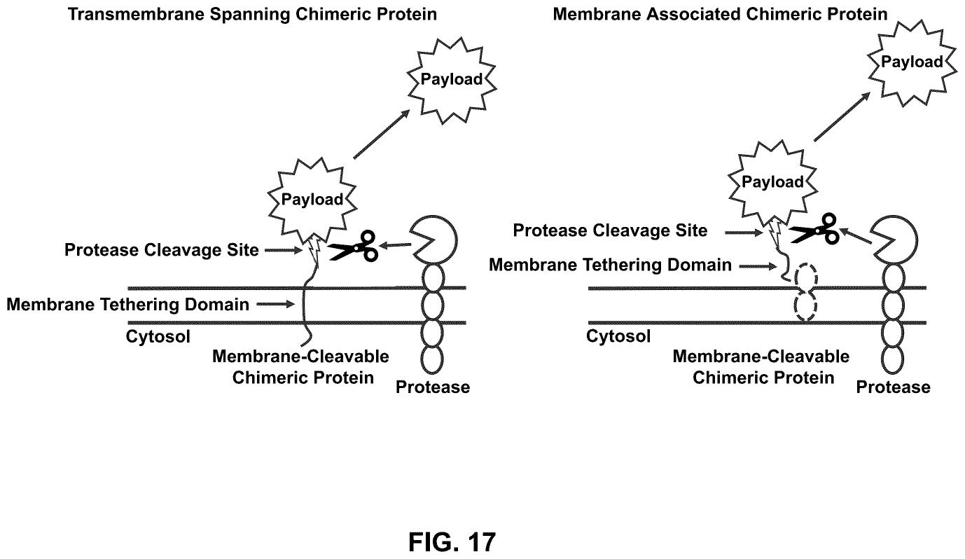

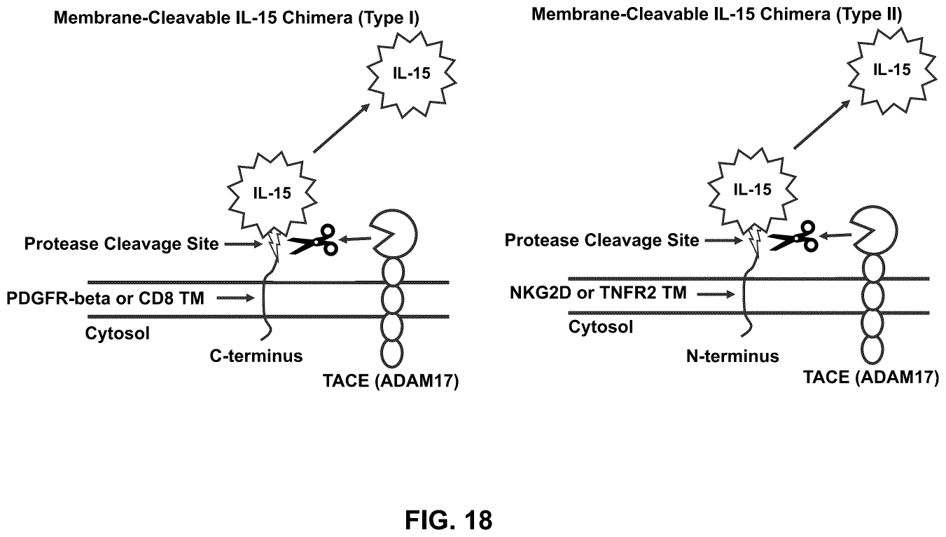

FIG. 17 illustrates a schematic of the Membrane-Cleavable system described herein in which a desired payload is expressed as a chimeric protein in which a protease cleavage site is inserted between the payload and membrane-tethering domain. The left panel illustrates a schematic of the Membrane-Cleavable system using a transmembrane spanning architecture (e.g., the chimeric protein contains a transmembrane domain). The right panel illustrates a schematic of the Membrane-Cleavable system using a membrane-associated architecture (e.g., the chimeric protein contains a post-translational modification tag allowing association with a cell membrane). FIG. 18 illustrates a representative Membrane-Cleavable system for the regulated secretion of IL-15 in which IL-15 is expressed as a chimeric protein having a cleavage site capable of cleavage by TACE inserted between the IL-15 payload and membrane-tethering domain. The left panel illustrates a Type I transmembrane architecture (e.g., S—C-MT) through use of Type I transmembrane domains, such as PDGFR-beta and CD8. The right panel a Type II transmembrane architecture (e.g., MT-C—S) through use of Type II transmembrane domains, such as NKG2D and TNFR2.

In some aspects, membrane-cleavable chimeric proteins (or engineered nucleic acids encoding the membrane-cleavable chimeric proteins) are provided for herein having a protein of interest (e.g., any of the effector molecules described herein), a protease cleavage site, and a cell membrane tethering domain.

An “effector molecule,” refers to a molecule (e.g., a nucleic acid such as DNA or RNA, or a protein (polypeptide) or peptide) that binds to another molecule and modulates the biological activity of that molecule to which it binds. For example, an effector molecule may act as a ligand to increase or decrease enzymatic activity, gene expression, or cell signaling. Thus, in some embodiments, an effector molecule modulates (activates or inhibits) different immunomodulatory mechanisms. By directly binding to and modulating a molecule, an effector molecule may also indirectly modulate a second, downstream molecule.

In certain embodiments described herein (e.g., in general, for all membrane-cleavable chimeric proteins described herein), an effector molecule is a secretable effector molecule (e.g., referred to as “S” in the formula S—C-MT or MT-C—S for membrane-cleavable chimeric proteins described herein). Non-limiting examples of effector molecules include cytokines, chemokines, enzymes that modulate metabolite levels, growth factors, co-activation molecules, tumor microenvironment modifiers, ligands, peptides, enzymes, antibodies, antibodies or decoy molecules that modulate cytokines, homing molecules, and/or integrins.

The term “modulate” encompasses maintenance of a biological activity, inhibition (partial or complete) of a biological activity, and stimulation/activation (partial or complete) of a biological activity. The term also encompasses decreasing or increasing (e.g., enhancing) a biological activity. Two different effector molecules are considered to “modulate different tumor-mediated immunosuppressive mechanisms” when one effector molecule modulates a tumor-mediated immunosuppressive mechanism (e.g., stimulates T cell signaling) that is different from the tumor-mediated immunosuppressive mechanism modulated by the other effector molecule (e.g., stimulates antigen presentation and/or processing).

Modulation by an effector molecule may be direct or indirect. Direct modulation occurs when an effector molecule binds to another molecule and modulates activity of that molecule. Indirect modulation occurs when an effector molecule binds to another molecule, modulates activity of that molecule, and as a result of that modulation, the activity of yet another molecule (to which the effector molecule is not bound) is modulated.

In some embodiments, modulation of a tumor-mediated immunosuppressive mechanism by at least one effector molecule results in an increase in an immunostimulatory and/or anti-tumor immune response (e.g., systemically or in the tumor microenvironment) by at least 10% (e.g., 10%, 20%, 30%, 40%, 50%, 60%, 70%, 80%, 90%, 100%, or 200%). For example, modulation of a tumor-mediated immunosuppressive mechanism may result in an increase in an immunostimulatory and/or anti-tumor immune response by at least 20%, at least 30%, at least 40%, at least 50%, at least 60%, at least 70%, at least 80%, at least 90%, at least 100%. In some embodiments, modulation of a tumor-mediated immunosuppressive mechanism results in an increase in an immunostimulatory and/or anti-tumor immune response 10-20%, 10-30%, 10-40%, 10-50%, 10-60%, 10-70%, 10-80%, 10-90%, 10-100%, 10-200%, 20-30%, 20-40%, 20-50%, 20-60%, 20-70%, 20-80%, 20-90%, 20-100%, 20-200%, 50-60%, 50-70%, 50-80%, 50-90%, 50-100%, or 50-200%. It should be understood that “an increase” in an immunostimulatory and/or anti-tumor immune response, for example, systemically or in a tumor microenvironment, is relative to the immunostimulatory and/or anti-tumor immune response that would otherwise occur, in the absence of the effector molecule(s).

In some embodiments, modulation of a tumor-mediated immunosuppressive mechanism by at least one effector molecule results in an increase in an immunostimulatory and/or anti-tumor immune response (e.g., systemically or in the tumor microenvironment) by at least 2 fold (e.g., 2, 3, 4, 5, 10, 25, 20, 25, 50, or 100 fold). For example, modulation of a tumor-mediated immunosuppressive mechanism may result in an increase in an immunostimulatory and/or anti-tumor immune response by at least 3 fold, at least 5 fold, at least 10 fold, at least 20 fold, at least 50 fold, or at least 100 fold. In some embodiments, modulation of a tumor-mediated immunosuppressive mechanism results in an increase in an immunostimulatory and/or anti-tumor immune response by 2-10, 2-20, 2-30, 2-40, 2-50, 2-60, 2-70, 2-80, 2-90, or 2-100 fold.

Non-limiting examples of immunostimulatory and/or anti-tumor immune mechanisms include T cell signaling, activity and/or recruitment, antigen presentation and/or processing, natural killer cell-mediated cytotoxic signaling, activity and/or recruitment, dendritic cell differentiation and/or maturation, immune cell recruitment, pro-inflammatory macrophage signaling, activity and/or recruitment, stroma degradation, immunostimulatory metabolite production, stimulator of interferon genes (STING) signaling (which increases the secretion of IFN and Th1 polarization, promoting an anti-tumor immune response), and/or Type I interferon signaling. An effector molecule may stimulate at least one (one or more) of the foregoing immunostimulatory mechanisms, thus resulting in an increase in an immunostimulatory response. Changes in the foregoing immunostimulatory and/or anti-tumor immune mechanisms may be assessed, for example, using in vitro assays for T cell proliferation or cytotoxicity, in vitro antigen presentation assays, expression assays (e.g., of particular markers), and/or cell secretion assays (e.g., of cytokines).

In some embodiments, modulation of a tumor-mediated immunosuppressive mechanism by at least one effector molecule results in a decrease in an immunosuppressive response (e.g., systemically or in the tumor microenvironment) by at least 10% (e.g., 10%, 20%, 30%, 40%, 50%, 60%, 70%, 80%, 90%, 100%, or 200%). For example, modulation of a tumor-mediated immunosuppressive mechanism may result in a decrease in an immunosuppressive response by at least 20%, at least 30%, at least 40%, at least 50%, at least 60%, at least 70%, at least 80%, at least 90%, at least 100%. In some embodiments, modulation of a tumor-mediated immunosuppressive mechanism results in a decrease in an immunosuppressive response 10-20%, 10-30%, 10-40%, 10-50%, 10-60%, 10-70%, 10-80%, 10-90%, 10-100%, 10-200%, 20-30%, 20-40%, 20-50%, 20-60%, 20-70%, 20-80%, 20-90%, 20-100%, 20-200%, 50-60%, 50-70%, 50-80%, 50-90%, 50-100%, or 50-200%. It should be understood that “a decrease” in an immunosuppressive response, for example, systemically or in a tumor microenvironment, is relative to the immunosuppressive response that would otherwise occur, in the absence of the effector molecule(s).

In some embodiments, modulation of a tumor-mediated immunosuppressive mechanism by at least one effector molecule results in a decrease in an immunosuppressive response (e.g., systemically or in the tumor microenvironment) by at least 2 fold (e.g., 2, 3, 4, 5, 10, 25, 20, 25, 50, or 100 fold). For example, modulation of a tumor-mediated immunosuppressive mechanism may result in a decrease in an immunosuppressive response by at least 3 fold, at least 5 fold, at least 10 fold, at least 20 fold, at least 50 fold, or at least 100 fold. In some embodiments, modulation of a tumor-mediated immunosuppressive mechanism results in a decrease in an immunosuppressive response by 2-10, 2-20, 2-30, 2-40, 2-50, 2-60, 2-70, 2-80, 2-90, or 2-100 fold.

Non-limiting examples of immunosuppressive mechanisms include negative costimulatory signaling, pro-apoptotic signaling of cytotoxic cells (e.g., T cells and/or NK cells), T regulatory (Treg) cell signaling, tumor checkpoint molecule production/maintenance, myeloid-derived suppressor cell signaling, activity and/or recruitment, immunosuppressive factor/metabolite production, and/or vascular endothelial growth factor signaling. An effector molecule may inhibit at least one (one or more) of the foregoing immunosuppressive mechanisms, thus resulting in a decrease in an immunosuppressive response. Changes in the foregoing immunosuppressive mechanisms may be assessed, for example, by assaying for an increase in T cell proliferation and/or an increase in IFNγ production (negative co-stimulatory signaling, T_{reg} cell signaling and/or MDSC); Annexin V/PI flow staining (pro-apoptotic signaling); flow staining for expression, e.g., PDL1 expression (tumor checkpoint molecule production/maintenance); ELISA, LUMINEX®, RNA via qPCR, enzymatic assays, e.g., IDO tryptophan catabolismimmunosuppressive factor/metabolite production); and phosphorylation of PI3K, Akt, p38 (VEGF signaling).

In some embodiments, effector molecules function additively: the effect of two effector molecules, for example, may be equal to the sum of the effect of the two effector molecules functioning separately. In other embodiments, effector molecules function synergistically: the effect of two effector molecules, for example, may be greater than the combined function of the two effector molecules.

Effector molecules that modulate tumor-mediated immunosuppressive mechanisms and/or modify tumor microenvironments may be, for example, secreted factors (e.g., cytokines, chemokines, antibodies, and/or decoy receptors that modulate extracellular mechanisms involved in the immune system), inhibitors (e.g., antibodies, antibody fragments, ligand TRAP and/or small blocking peptides), intracellular factors that control cell state (e.g., microRNAs and/or transcription factors that modulate the state of cells to enhance pro-inflammatory properties), factors packaged into exosomes (e.g., microRNAs, cytosolic factors, and/or extracellular factors), surface displayed factors (e.g., checkpoint inhibitors, TRAIL), and and/or metabolic genes (e.g., enzymes that produce/modulate or degrade metabolites or amino acids).

In some embodiments, at least one of the effector molecules stimulates an immunostimulatory mechanism in the tumor microenvironment and/or inhibits an immunosuppressive mechanism in the tumor microenvironment.

In some embodiments, at least one of the effector molecules (a) stimulates T cell signaling, activity and/or recruitment, (b) stimulates antigen presentation and/or processing, (c) stimulates natural killer cell-mediated cytotoxic signaling, activity and/or recruitment, (d) stimulates dendritic cell differentiation and/or maturation, (c) stimulates immune cell recruitment, (f) stimulates pro-inflammatory macrophage signaling, activity and/or recruitment or inhibits anti-inflammatory macrophage signaling, activity and/or recruitment, (g) stimulates stroma degradation, (h) stimulates immunostimulatory metabolite production, (i) stimulates Type I interferon signaling, (j) inhibits negative costimulatory signaling, (k) inhibits pro-apoptotic signaling of anti-tumor immune cells, (l) inhibits T regulatory (T_{reg}) cell signaling, activity and/or recruitment, (m) inhibits tumor checkpoint molecules, (n) stimulates stimulator of interferon genes (STING) signaling, (o) inhibits myeloid-derived suppressor cell signaling, activity and/or recruitment, (p) degrades immunosuppressive factors/metabolites, (q) inhibits vascular endothelial growth factor signaling, and/or (r) directly kills tumor cells.

In some embodiments, effector molecules may be selected from the following non-limiting classes of molecules: cytokines, antibodies, chemokines, nucleotides, peptides, and enzymes. Non-limiting examples of the foregoing classes of effector molecules are listed in Table 1 and specific sequences encoding exemplary effector molecules are listed in Table 2. Effector molecules can be human, such as those listed in Table 1 or Table 2 or human equivalents of murine effector molecules listed in Table 1 or Table 2. Effector molecules can be human-derived, such as the endogenous human effector molecule or an effector molecule modified and/or optimized for function, e.g., codon optimized to improve expression, modified to improve stability, or modified at its signal sequence (see below). Various programs and algorithms for optimizing function are known to those skilled in the art and can be selected based on the improvement desired, such as codon optimization for a specific species (e.g., human, mouse, bacteria, etc.).

In some embodiments, the effector molecule comprises interleukin 12 (IL-12), for example, p35 and p40 as a dimer that is generally referred to in the art as IL12p70. In some embodiments, the first effector molecule comprises an IL12p70 fusion protein. In some embodiments, the IL12p70 fusion protein is a human IL12p70 fusion protein. In some embodiments, the human IL12p70 fusion protein comprises the sequence shown in SEQ ID NO: 203.

In some embodiments, the effector molecule comprises interleukin 15 (IL-15). In some embodiments, the effector molecule consists of IL-15 (see, e.g., SEQ ID NO: 199). In some embodiments, the effector molecule comprises a fusion protein including IL-15 and an extracellular portion of IL-15 Receptor a (IL-15Ra), such as the sushi domain as shown in SEQ ID NO: 201. An exemplary IL-15/IL-15Ra sushi domain fusion is provided as SEQ ID NO: 202. In some embodiments, the effector molecule includes IL-15 having the amino acid sequence NWVNVISDLKKIEDLIQSMHIDATLYTESDVHPSCKVTAMKCFLLELQVISLESGDASIH DTVENLIILANNSLSSNGNVTESGCKECEELEEKNIKEFLQSFVHIVQMFINTS (SEQ ID NO: 224). In some embodiments, IL-15 is encoded by a polynucleotide sequence comprising the sequence AATTGGGTCAACGTGATCAGCGACCTGAAGAAGATCGAGGACCTGATCCAGAGCAT GCACATCGACGCCACACTGTACACCGAGTCCGATGTGCACCCTAGCTGCAAAGTGA CCGCCATGAAGTGCTTTCTGCTGGAACTGCAAGTGATCAGCCTGGAAAGCGGCGAC GCCAGCATCCACGATACCGTGGAAAATCTGATCATCCTGGCCAACAACAGCCTGTC CAGCAACGGCAATGTGACCGAGAGCGGCTGCAAAGAGTGCGAGGAACTGGAAGAG AAGAACATCAAAGAGTTTCTGCAGAGCTTCGTCCACATCGTGCAGATGTTCATCAA CACCTCA (SEQ ID NO: 225). In some embodiments, IL-15 is encoded by a polynucleotide sequence that includes a sequence that is at least 90%, at least 91%, at least 92%, at least 93%, at least 94%, at least 95%, at least 96%, at least 97%, at least 98%, or at least 99% identical to

(SEQ ID NO: 225)
AATTGGGTCAACGTGATCAGCGACCTGAAGAAGATCGAGGACCTG
ATCCAGAGCATGCACATCGACGCCACACTGTACACCGAGTCCGAT
GTGCACCCTAGCTGCAAAGTGACCGCCATGAAGTGCTTTCTGCTG
GAACTGCAAGTGATCAGCCTGGAAAGCGGCGACGCCAGCATCCAC
GATACCGTGGAAAATCTGATCATCCTGGCCAACAACAGCCTGTCC
AGCAACGGCAATGTGACCGAGAGCGGCTGCAAAGAGTGCGAGGAA
CTGGAAGAGAAGAACATCAAAGAGTTTCTGCAGAGCTTCGTCCAC
ATCGTGCAGATGTTCATCAACACCTCA.

In some embodiments, the membrane-cleavable chimeric protein includes the amino acid sequence MDWTWILFLVAAATRVHSNWVNVISDLKKIEDLIQSMHIDATLYTESDVHPSCKVTAM KCFLLELQVISLESGDASIHDTVENLIILANNSLSSNGNVTESGCKECEELEEKNIKEFLQS FVHIVQMFINTSSGGGGSGGGGSGVTPEPIFSLIGGGSGGGGSGGGSLQLLPSWAITLISV NGIFVICCLTYCFAPRCRERRRNERLRRESVRPV (SEQ ID NO: 226). In some embodiments, the membrane-cleavable chimeric protein is encoded by a polynucleotide sequence comprising the sequence ATGGACTGGACTTGGATACTCTTTCTGGTCGCTGCCGCCACACGGGTGCACTCTAAT TGGGTCAACGTGATCAGCGACCTGAAGAAGATCGAGGACCTGATCCAGAGCATGCA CATCGACGCCACACTGTACACCGAGTCCGATGTGCACCCTAGCTGCAAAGTGACCG CCATGAAGTGCTTTCTGCTGGAACTGCAAGTGATCAGCCTGGAAAGCGGCGACGCC AGCATCCACGATACCGTGGAAAATCTGATCATCCTGGCCAACAACAGCCTGTCCAG CAACGGCAATGTGACCGAGAGCGGCTGCAAAGAGTGCGAGGAACTGGAAGAGAAG AACATCAAAGAGTTTCTGCAGAGCTTCGTCCACATCGTGCAGATGTTCATCAACACC TCATCAGGCGGCGGTGGTAGTGGAGGCGGAGGCTCAGGCGTGACCCCTGAGCCTAT CTTCAGCCTGATCGGCGGAGGTTCCGGAGGTGGCGGTTCCGGCGGAGGATCTCTTC AATTGCTGCCTAGCTGGGCCATCACACTGATCTCCGTGAACGGCATCTTCGTGATCT GCTGCCTGACCTACTGCTTCGCCCCTAGATGCAGAGAGCGGAGAAGAAACGAGCGG CTGAGAAGAGAAAGCGTGCGGCCTGTG (SEQ ID NO: 227). In some embodiments, the membrane-cleavable chimeric protein is encoded by a polynucleotide sequence that includes a sequence that is at least 90%, at least 91%, at least 92%, at least 93%, at least 94%, at least 95%, at least 96%, at least 97%, at least 98%, or at least 99% identical to

(SEQ ID NO: 227)
ATGGACTGGACTTGGATACTCTTTCTGGTCGCTGCCGCCACACGG
GTGCACTCTAATTGGGTCAACGTGATCAGCGACCTGAAGAAGATC
GAGGACCTGATCCAGAGCATGCACATCGACGCCACACTGTACACC
GAGTCCGATGTGCACCCTAGCTGCAAAGTGACCGCCATGAAGTGC
TTTCTGCTGGAACTGCAAGTGATCAGCCTGGAAAGCGGCGACGCC
AGCATCCACGATACCGTGGAAAATCTGATCATCCTGGCCAACAAC
AGCCTGTCCAGCAACGGCAATGTGACCGAGAGCGGCTGCAAAGAG
TGCGAGGAACTGGAAGAGAAGAACATCAAAGAGTTTCTGCAGAGC
TTCGTCCACATCGTGCAGATGTTCATCAACACCTCATCAGGCGGC
GGTGGTAGTGGAGGCGGAGGCTCAGGCGTGACCCCTGAGCCTATC
TTCAGCCTGATCGGCGGAGGTTCCGGAGGTGGCGGTTCCGGCGGA
GGATCTCTTCAATTGCTGCCTAGCTGGGCCATCACACTGATCTCC
GTGAACGGCATCTTCGTGATCTGCTGCCTGACCTACTGCTTCGCC
CCTAGATGCAGAGAGCGGAGAAGAAACGAGCGGCTGAGAAGAGAA
AGCGTGCGGCCTGTG.

TABLE 1
Exemplary Effector Molecules

| Effector name | Category | Function |
|---|---|---|
| anti-CD40 or CD40 | Agonist antibody | Stimulates B-cells and |
| Ligand | | antigen presenting cells. |
| Flt3L | Ligand agonist | Stimulates myeloid |
| | | cells and antigen |
| | | presenting cells |
| CXCL10-11 fusion | Chemokine | Attracts T-cells |
| TGFb blocking | Antagonist peptides | Inhibit TGFb pathway, |
| peptides | | TME modifier |
| Adenosine | TME modifier | Degradation of |
| deaminase | | suppressive adenosine in |
| (ADA) | | the TME |
| Kyneurinase | TME modifier | Degradation of kyneurine |
| HPGE2 | TME modifier | Degradation of PGE2 |
| CXCL13 | Chemokine | Attracts B-cells |
| anti PD-1/PD-L1 | Agonist antibody | Remove checkpoint |
| anti-CTLA-4 | Agonist antibody | Remove checkpoint |
| anti-VEGF | Antagonist | Neutralizes an |
| | antibody | immunosuppressive/ |
| | | angiogenesis factor |
| anti-TNFa | Antagonist | Neutralizes cytokine/ |
| | antibody | pro-tumor factor |
| anti-IL-10 | Antagonist | Neutralizes |
| | antibody | immunosuppressive |
| | | cytokine |
| anti-SDF1/ | Antagonist | Neutralizes pro-tumor |
| CXCL12 | | chemokine |
| | antibody | |
| (TβRII)2 trap | Capture trap | Neutralizes an |
| | | immunosuppressive |
| | | cytokine |
| CCL21 | Chemokine | Attracts leukocytes/NK |
| CCL1 | Chemokine | Attracts leukocytes/NK |
| CCL17 | Chemokine | Attracts leukocytes/NK |
| CCL19 | Chemokine | Attracts leukocytes/NK |
| CCL21 | Chemokine | Attracts leukocytes/NK |
| CCL20 | Chemokine | Attracts leukocytes/NK |
| CCL21a | Chemokine | Attracts leukocytes/NK |
| MIP1b (CCL5) | Chemokine | Attracts leukocytes/NK |
| CXCL10 | Chemokine | Attracts leukocytes/NK |
| CXCL11 | Chemokine | Attracts leukocytes/NK |
| CCL2 | Chemokine | Attracts monocytes |
| MIP-1 alpha | Chemokine | Attracts leukocytes/NK |
| (CCL3) | | |
| XCL1 | Chemokine | Attracts leukocytes/NK |
| IFNbeta | Cytokine | T cell response, tumor |
| | | cell killing |
| IFNgamma | Cytokine | T cell response, tumor |
| | | cell killing |
| IL-12 | Cytokine | T cells, NK cells |
| IL-1beta | Cytokine | T cells, NK cells |
| IL-15 | Cytokine | Stimulates T-cells and NK |
| IL-2 | Cytokine | Stimulates T-cells and NK |
| IL-21 | Cytokine | Stimulates T-cells |
| IL-24 | Cytokine | Stimulates T-cells |
| IL36-gamma | Cytokine | Stimulates T-cells |
| IL-7 | Cytokine | Stimulates T-cells |
| IL-22 | Cytokine | Stimulates T-cells |
| IL-18 | Cytokine | Stimulates T-cells |
| Granzymes/Perforin | Enzyme | Direct tumor cell killing |
| OX86 (anti-OX40) | ligand | Stimulates T-cells |
| anti-TGFbeta | Neutralizing | Neutralizes an |
| | antibody | Immunosuppressive |
| | | cytokine |
| TRAIL | Receptor/ligand | Direct tumor cell killing |
| FASL (CD49L) | Receptor/ligand | Direct tumor cell killing |
| OX40-L | Receptor/Ligand | Stimulates T-cells |
| cGAS | secreted molecule | Stimulates antigen- |
| | | presenting cells |
| 41BBL | secreted molecule | Co-activation of T-cells |
| CD40L | secreted molecule | Stimulates T-cells |
| GM-CSF | secreted molecule | Growth factor for monocytes |
| STING | secreted molecule | Stimulates antigen- |
| | | presenting cells |
| HAC-V | Antagonist | inhibits checkpoint |
| ‘microbody’_PD1 | antibody | |
| yCD | Pro-drug | Converts to cytotoxic |
| | | molecule upon |
| | | activation |
| CpG/Nucleotides | Nucleotides | STING agonist |

TABLE 2
Exemplary effector molecule sequences

IL-12 (Human) (SEQ ID NO: 56)
ATGTGCCATCAGCAGCTTGTCATATCTTGGTTTTCACTTGTATTCCTGGCCAGCCCTTTGGTTGCG
ATCTGGGAGCTCAAGAAGGATGTGTACGTTGTAGAGCTGGACTGGTACCCCGATGCTCCCGGTG
AGATGGTCGTTTTGACATGTGACACTCCAGAAGAGGACGGTATTACGTGGACTCTGGACCAGTC
CTCCGAAGTTCTTGGTTCTGGTAAGACTCTGACTATCCAGGTGAAAGAATTTGGGGATGCGGGAC
AATACACATGCCACAAGGGAGGCGAGGTGTTGTCTCATAGTTTGCTGCTTCTCCACAAGAAAGA
GGATGGAATCTGGAGCACCGACATACTCAAGGATCAAAAGGAACCCAAAAATAAGACATTTCTG
CGATGTGAGGCTAAGAACTATAGTGGCCGCTTCACTTGTTGGTGGCTGACTACCATCAGCACAG
ATCTCACGTTTTCAGTAAAAAGTAGTAGAGGTTCAAGTGATCCTCAAGGGGTAACGTGCGGTGC
TGCAACACTGTCTGCTGAACGCGTAAGAGGAGATAATAAGGAGTACGAGTATTCCGTAGAATGC
CAAGAGGACAGTGCTTGTCCTGCGGCCGAGGAGTCTCTCCCAATAGAAGTGATGGTGGACGCGG
TGCATAAACTGAAATATGAGAACTACACAAGCAGTTTTTTTATAAGAGATATCATCAAGCCCGA
TCCGCCGAAGAATTTGCAACTTAAACCGCTTAAAAACTCACGCCAGGTTGAAGTATCCTGGGAG
TATCCGGATACATGGTCAACACCACACAGCTATTTTTCCCTTACCTTCTGTGTGCAGGTCCAAGG
GAAGAGCAAAAGGGAGAAGAAGGACAGGGTATTCACTGATAAAACTTCCGCGACGGTCATCTG
CCGAAAAAACGCTAGTATATCTGTACGGGCGCAGGATAGGTACTATAGTTCTTCTTGGTCTGAGT
GGGCCTCAGTTCCGTGCTCTGGGGGAGGAAGTGGAGGAGGGTCCGGCGGTGGAAGCGGGGGAG
GGAGTCGCAACTTGCCAGTGGCTACACCAGATCCAGGCATGTTTCCATGTCTGCATCATTCCCAG
AATCTCCTGAGAGCGGTGTCAAATATGCTCCAAAAAGCGAGACAAACACTGGAATTTTACCCGT
GTACCAGTGAGGAGATTGATCACGAGGACATAACCAAGGACAAGACCTCAACTGTAGAAGCGT
GTTTGCCGCTGGAGTTGACTAAGAATGAGTCCTGCCTCAATTCCAGAGAAACTTCATTCATTACT
AACGGCAGTTGTCTTGCATCCCGGAAAACGTCCTTTATGATGGCCCTTTGCCTTAGTTCAATTTAC
GAGGATCTTAAAATGTATCAAGTGGAGTTTAAAACCATGAATGCTAAACTTCTTATGGACCCCA
AACGACAAATTTTTCTGGATCAGAATATGCTTGCCGTGATAGACGAACTCATGCAGGCGCTTAAT
TTTAACTCCGAAACAGTTCCACAAAAATCTAGCCTTGAAGAACCTGATTTTTATAAAACGAAGAT
TAAACTGTGTATCCTGCTGCATGCCTTTCGCATCCGAGCTGTCACAATCGATAGGGTTATGTCCT
ACCTTAACGCGAGCtaG
IL-12p70 (Human; codon optimized; bold denotes signal sequence) (SEQ ID NO: 57)
ATGTGCCATCAGCAACTCGTCATCTCCTGGTTCTCCCTTGTGTTCCTCGCTTCCCCTCTGGT
CGCCATTTGGGAACTGAAGAAGGACGTCTACGTGGTCGAGCTGGATTGGTACCCGGACGCCCCT
GGAGAAATGGTCGTGCTGACTTGCGATACGCCAGAAGAGGACGGCATAACCTGGACCCTGGATC
AGAGCTCCGAGGTGCTCGGAAGCGGAAAGACCCTGACCATTCAAGTCAAGGAGTTCGGCGACGC
GGGCCAGTACACTTGCCACAAGGGTGGCGAAGTGCTGTCCCACTCCCTGCTGCTGCTGCACAAG
AAAGAGGATGGAATCTGGTCCACTGACATCCTCAAGGACCAAAAAGAACCGAAGAACAAGACC
TTCCTCCGCTGCGAAGCCAAGAACTACAGCGGTCGGTTCACCTGTTGGTGGCTGACGACAATCTC
CACCGACCTGACTTTCTCCGTGAAGTCGTCACGGGGATCAAGCGATCCTCAGGGCGTGACCTGTG
GAGCCGCCACTCTGTCCGCCGAGAGAGTCAGGGGAGACAACAAGGAATATGAGTACTCCGTGG
AATGCCAGGAGGACAGCGCCTGCCCTGCCGCGGAAGAGTCCCTGCCTATCGAGGTCATGGTCGA
TGCCGTGCATAAGCTGAAATACGAGAACTACACTTCCTCCTTCTTTATCCGCGACATCATCAAGC
CTGACCCCCCCAAGAACTTGCAGCTGAAGCCACTCAAGAACTCCCGCCAAGTGGAAGTGTCTTG
GGAATATCCAGACACTTGGAGCACCCCGCACTCATACTTCTCGCTCACTTTCTGTGTGCAAGTGC
AGGGAAAGTCCAAACGGGAGAAGAAAGACCGGGTGTTCACCGACAAAACCTCCGCCACTGTGA
TTTGTCGGAAGAACGCGTCAATCAGCGTCCGGGCGCAGGATAGATACTACTCGTCCTCCTGGAG
CGAATGGGCCAGCGTGCCTTGTTCCGGTGGCGGATCAGGCGGAGGTTCAGGAGGAGGCTCCGGA
GGAGGTTCCCGGAACCTCCCTGTGGCAACCCCCGACCCTGGAATGTTCCCGTGCCTACACCACTC
CCAAAACCTCCTGAGGGCTGTGTCGAACATGTTGCAGAAGGCCCGCCAGACCCTTGAGTTCTAC
CCCTGCACCTCGGAAGAAATTGATCACGAGGACATCACCAAGGACAAGACCTCGACCGTGGAAG
CCTGCCTGCCGCTGGAACTGACCAAGAACGAATCGTGTCTGAACTCCCGCGAGACAAGCTTTAT
CACTAACGGCAGCTGCCTGGCGTCGAGAAAGACCTCATTCATGATGGCGCTCTGTCTTTCCTCGA
TCTACGAAGATCTGAAGATGTATCAGGTCGAGTTCAAGACCATGAACGCCAAGCTGCTCATGGA
CCCGAAGCGGCAGATCTTCCTGGACCAGAATATGCTCGCCGTGATTGATGAACTGATGCAGGCC
CTGAATTTCAACTCCGAGACTGTGCCTCAAAAGTCCAGCCTGGAAGAACCGGACTTCTACAAGA
CCAAGATCAAGCTGTGCATCCTGTTGCACGCTTTCCGCATTCGAGCCGTGACCATTGACCGCGTG
ATGTCCTACCTGAACGCCAGT
IL-12 (Mouse) (SEQ ID NO: 58)
ATGTGTCCACAGAAGCTGACAATAAGTTGGTTTGCCATTGTCCTCCTGGTGAGCCCACTCATGGC
AATGTGGGAACTCGAAAAGGATGTCTACGTGGTAGAAGTAGATTGGACTCCAGACGCGCCAGGG
GAGACAGTGAATTTGACATGTGACACACCAGAAGAAGATGACATTACATGGACATCTGACCAAC
GCCATGGCGTAATAGGGAGTGGGAAAACACTCACGATCACAGTTAAAGAGTTCTTGGATGCTGG
TCAATATACTTGCCATAAAGGCGGCGAGACACTCAGCCACTCACATTTGCTTTTGCATAAAAAAG
AGAATGGCATTTGGAGCACTGAAATACTTAAGAACTTTAAGAACAAGACATTTCTCAAGTGTGA
GGCCCCTAATTACAGCGGCAGGTTCACGTGCTCATGGCTGGTCCAGCGCAACATGGACCTCAAG
TTTAACATAAAATCTTCTTCCTCTTCACCTGACTCCAGAGCTGTTACTTGCGGCATGGCTTCTCTG
AGCGCAGAAAAAGTAACGTTGGATCAAAGAGACTACGAAAAGTACTCTGTTTCTTGTCAAGAGG
ATGTTACGTGCCCGACGGCCGAAGAAACGCTTCCAATTGAACTCGCGTTGGAAGCTCGCCAACA
AAACAAGTATGAAAACTACAGTACAAGCTTCTTTATACGGGATATAATTAAACCCGATCCCCCC
AAGAACTTGCAAATGAAACCACTTAAGAACAGCCAGGTGGAAGTTTCCTGGGAGTATCCAGACT
CATGGAGTACTCCTCACAGCTATTTTTCTCTGAAATTCTTTGTAAGGATACAACGGAAGAAAGAG
AAGATGAAAGAGACCGAGGAGGGTTGTAATCAGAAGGGAGCGTTTCTCGTGGAGAAAACGTCT
ACCGAAGTCCAATGTAAAGGTGGCAATGTGTGCGTCCAAGCTCAGGATAGATACTATAATTCAA
GTTGCTCCAAGTGGGCCTGTGTTCCATGCCGCGTTCGGAGCGGGGGAGGTAGCGGAGGAGGTAG
TGGGGGTGGGTCAGGAGGAGGGAGTCGAGTTATCCCGGTGTCAGGCCCCGCACGCTGCTTGAGC
CAGAGTCGCAACCTCCTTAAGACAACAGATGACATGGTGAAAACAGCACGCGAAAAGCTTAAA
CACTACTCTTGTACGGCGGAGGATATTGATCACGAGGATATTACCCGAGACCAAACTAGCACTTT
GAAAACCTGTCTGCCCCTTGAACTTCATAAAAATGAGAGCTGTCTGGCTACACGAGAGACGTCA
AGTACGACTAGGGGCAGCTGTCTCCCGCCGCAAAAGACAAGCCTCATGATGACGCTCTGTTTGG
GTTCCATTTACGAGGACTTGAAAATGTATCAAACGGAGTTCCAGGCTATAAATGCGGCGTTGCA
GAACCATAACCATCAACAAATTATACTTGATAAAGGCATGTTGGTGGCGATTGATGAACTCATG
CAGAGTCTCAATCACAACGGGGAAACGTTGAGACAGAAACCCCCAGTCGGTGAAGCGGACCCA
TATCGAGTAAAAATGAAGCTCTGCATTCTGCTTCACGCATTCAGCACTAGAGTTGTTACCATCAA
CCGGGTAATGGGATATCTCTCCAGTGCGtaG
IL21 (Human; codon optimized; bold denotes signal sequence) (SEQ ID NO: 59)
ATGGAACGCATTGTGATCTGCCTGATGGTCATCTTCCTGGGCACCTTAGTGCACAAGTCGA
GCAGCCAGGGACAGGACAGGCACATGATTAGAATGCGCCAGCTCATCGATATCGTGGACCAGT
TGAAGAACTACGTGAACGACCTGGTGCCCGAGTTCCTGCCGGCCCCCGAAGATGTGGAAACCAA
TTGCGAATGGTCGGCATTTTCCTGCTTTCAAAAGGCACAGCTCAAGTCCGCTAACACCGGGAACA
ACGAACGGATCATCAACGTGTCCATCAAAAAGCTGAAGCGGAAGCCTCCCTCCACCAACGCCGG
ACGGAGGCAGAAGCATAGGCTGACTTGCCCGTCATGCGACTCCTACGAGAAGAAGCCGCCGAA
GGAGTTCCTGGAGCGGTTCAAGTCGCTCCTGCAAAAGATGATTCATCAGCACCTGTCCTCCCGGA
CTCATGGGTCTGAGGATTCA
IL-12p70_T2A_IL21 (Human; codon optimized; bold denotes signal sequences) (SEQ ID NO: 60)
ATGTGCCATCAGCAACTCGTCATCTCCTGGTTCTCCCTTGTGTTCCTCGCTTCCCCTCTGGT
CGCCATTTGGGAACTGAAGAAGGACGTCTACGTGGTCGAGCTGGATTGGTACCCGGACGCCCCT
GGAGAAATGGTCGTGCTGACTTGCGATACGCCAGAAGAGGACGGCATAACCTGGACCCTGGATC
AGAGCTCCGAGGTGCTCGGAAGCGGAAAGACCCTGACCATTCAAGTCAAGGAGTTCGGCGACGC
GGGCCAGTACACTTGCCACAAGGGTGGCGAAGTGCTGTCCCACTCCCTGCTGCTGCTGCACAAG
AAAGAGGATGGAATCTGGTCCACTGACATCCTCAAGGACCAAAAAGAACCGAAGAACAAGACC
TTCCTCCGCTGCGAAGCCAAGAACTACAGCGGTCGGTTCACCTGTTGGTGGCTGACGACAATCTC
CACCGACCTGACTTTCTCCGTGAAGTCGTCACGGGGATCAAGCGATCCTCAGGGCGTGACCTGTG
GAGCCGCCACTCTGTCCGCCGAGAGAGTCAGGGGAGACAACAAGGAATATGAGTACTCCGTGG
AATGCCAGGAGGACAGCGCCTGCCCTGCCGCGGAAGAGTCCCTGCCTATCGAGGTCATGGTCGA
TGCCGTGCATAAGCTGAAATACGAGAACTACACTTCCTCCTTCTTTATCCGCGACATCATCAAGC
CTGACCCCCCCAAGAACTTGCAGCTGAAGCCACTCAAGAACTCCCGCCAAGTGGAAGTGTCTTG
GGAATATCCAGACACTTGGAGCACCCCGCACTCATACTTCTCGCTCACTTTCTGTGTGCAAGTGC
AGGGAAAGTCCAAACGGGAGAAGAAAGACCGGGTGTTCACCGACAAAACCTCCGCCACTGTGA
TTTGTCGGAAGAACGCGTCAATCAGCGTCCGGGCGCAGGATAGATACTACTCGTCCTCCTGGAG
CGAATGGGCCAGCGTGCCTTGTTCCGGTGGCGGATCAGGCGGAGGTTCAGGAGGAGGCTCCGGA
GGAGGTTCCCGGAACCTCCCTGTGGCAACCCCCGACCCTGGAATGTTCCCGTGCCTACACCACTC
CCAAAACCTCCTGAGGGCTGTGTCGAACATGTTGCAGAAGGCCCGCCAGACCCTTGAGTTCTAC
CCCTGCACCTCGGAAGAAATTGATCACGAGGACATCACCAAGGACAAGACCTCGACCGTGGAAG
CCTGCCTGCCGCTGGAACTGACCAAGAACGAATCGTGTCTGAACTCCCGCGAGACAAGCTTTAT
CACTAACGGCAGCTGCCTGGCGTCGAGAAAGACCTCATTCATGATGGCGCTCTGTCTTTCCTCGA
TCTACGAAGATCTGAAGATGTATCAGGTCGAGTTCAAGACCATGAACGCCAAGCTGCTCATGGA
CCCGAAGCGGCAGATCTTCCTGGACCAGAATATGCTCGCCGTGATTGATGAACTGATGCAGGCC
CTGAATTTCAACTCCGAGACTGTGCCTCAAAAGTCCAGCCTGGAAGAACCGGACTTCTACAAGA
CCAAGATCAAGCTGTGCATCCTGTTGCACGCTTTCCGCATTCGAGCCGTGACCATTGACCGCGTG
ATGTCCTACCTGAACGCCAGTAGACGGAAACGCGGAAGCGGAGAGGGCAGAGGCTCGCTGCTT
ACATGCGGGGACGTGGAAGAGAACCCCGGTCCGATGGAACGCATTGTGATCTGCCTGATGGT
CATCTTCCTGGGCACCTTAGTGCACAAGTCGAGCAGCCAGGGACAGGACAGGCACATGATTA
GAATGCGCCAGCTCATCGATATCGTGGACCAGTTGAAGAACTACGTGAACGACCTGGTGCCCGA
GTTCCTGCCGGCCCCCGAAGATGTGGAAACCAATTGCGAATGGTCGGCATTTTCCTGCTTTCAAA
AGGCACAGCTCAAGTCCGCTAACACCGGGAACAACGAACGGATCATCAACGTGTCCATCAAAAA
GCTGAAGCGGAAGCCTCCCTCCACCAACGCCGGACGGAGGCAGAAGCATAGGCTGACTTGCCCG
TCATGCGACTCCTACGAGAAGAAGCCGCCGAAGGAGTTCCTGGAGCGGTTCAAGTCGCTCCTGC
AAAAGATGATTCATCAGCACCTGTCCTCCCGGACTCATGGGTCTGAGGATTCA
IL-12_2A_CCL21a (Human) (SEQ ID NO: 61)
ATGTGCCATCAGCAGCTTGTCATATCTTGGTTTTCACTTGTATTCCTGGCCAGCCCTTTGGTTGCG
ATCTGGGAGCTCAAGAAGGATGTGTACGTTGTAGAGCTGGACTGGTACCCCGATGCTCCCGGTG
AGATGGTCGTTTTGACATGTGACACTCCAGAAGAGGACGGTATTACGTGGACTCTGGACCAGTC
CTCCGAAGTTCTTGGTTCTGGTAAGACTCTGACTATCCAGGTGAAAGAATTTGGGGATGCGGGAC
AATACACATGCCACAAGGGAGGCGAGGTGTTGTCTCATAGTTTGCTGCTTCTCCACAAGAAAGA
GGATGGAATCTGGAGCACCGACATACTCAAGGATCAAAAGGAACCCAAAAATAAGACATTTCTG
CGATGTGAGGCTAAGAACTATAGTGGCCGCTTCACTTGTTGGTGGCTGACTACCATCAGCACAG
ATCTCACGTTTTCAGTAAAAAGTAGTAGAGGTTCAAGTGATCCTCAAGGGGTAACGTGCGGTGC
TGCAACACTGTCTGCTGAACGCGTAAGAGGAGATAATAAGGAGTACGAGTATTCCGTAGAATGC
CAAGAGGACAGTGCTTGTCCTGCGGCCGAGGAGTCTCTCCCAATAGAAGTGATGGTGGACGCGG
TGCATAAACTGAAATATGAGAACTACACAAGCAGTTTTTTTATAAGAGATATCATCAAGCCCGA
TCCGCCGAAGAATTTGCAACTTAAACCGCTTAAAAACTCACGCCAGGTTGAAGTATCCTGGGAG
TATCCGGATACATGGTCAACACCACACAGCTATTTTTCCCTTACCTTCTGTGTGCAGGTCCAAGG
GAAGAGCAAAAGGGAGAAGAAGGACAGGGTATTCACTGATAAAACTTCCGCGACGGTCATCTG
CCGAAAAAACGCTAGTATATCTGTACGGGCGCAGGATAGGTACTATAGTTCTTCTTGGTCTGAGT
GGGCCTCAGTTCCGTGCTCTGGGGGAGGAAGTGGAGGAGGGTCCGGCGGTGGAAGCGGGGGAG
GGAGTCGCAACTTGCCAGTGGCTACACCAGATCCAGGCATGTTTCCATGTCTGCATCATTCCCAG
AATCTCCTGAGAGCGGTGTCAAATATGCTCCAAAAAGCGAGACAAACACTGGAATTTTACCCGT
GTACCAGTGAGGAGATTGATCACGAGGACATAACCAAGGACAAGACCTCAACTGTAGAAGCGT
GTTTGCCGCTGGAGTTGACTAAGAATGAGTCCTGCCTCAATTCCAGAGAAACTTCATTCATTACT
AACGGCAGTTGTCTTGCATCCCGGAAAACGTCCTTTATGATGGCCCTTTGCCTTAGTTCAATTTAC
GAGGATCTTAAAATGTATCAAGTGGAGTTTAAAACCATGAATGCTAAACTTCTTATGGACCCCA
AACGACAAATTTTTCTGGATCAGAATATGCTTGCCGTGATAGACGAACTCATGCAGGCGCTTAAT
TTTAACTCCGAAACAGTTCCACAAAAATCTAGCCTTGAAGAACCTGATTTTTATAAAACGAAGAT
TAAACTGTGTATCCTGCTGCATGCCTTTCGCATCCGAGCTGTCACAATCGATAGGGTTATGTCCT
ACCTTAACGCGAGCCGGCGCAAGAGGGGTTCCGGAGAGGGAAGGGGTAGTCTGCTCACCTGCG
GCGATGTTGAAGAAAATCCTGGTCCCATGGCGCAAAGTCTGGCTCTTTCACTCCTGATCCTGGTC
TTGGCCTTCGGGATTCCGAGGACCCAAGGAAGTGATGGTGGCGCCCAAGATTGTTGCCTTAAAT
ACAGCCAGCGGAAAATACCCGCGAAAGTGGTCAGGAGTTATAGAAAACAGGAGCCTTCCCTGG
GTTGTAGTATCCCCGCCATACTTTTCCTCCCGAGAAAACGGAGCCAGGCCGAACTGTGCGCTGAC
CCTAAGGAACTTTGGGTGCAACAACTTATGCAACACCTGGATAAGACACCTTCTCCTCAAAAGC
CAGCTCAGGGCTGCCGAAAAGATAGAGGCGCCTCAAAAACCGGAAAAAAGGGCAAAGGTTCTA
AAGGATGTAAGCGGACTGAACGCTCTCAAACGCCTAAAGGGCCGtaG
IL-12_2A_CCL21a (Mouse) (SEQ ID NO: 62)
ATGTGTCCACAGAAGCTGACAATAAGTTGGTTTGCCATTGTCCTCCTGGTGAGCCCACTCATGGC
AATGTGGGAACTCGAAAAGGATGTCTACGTGGTAGAAGTAGATTGGACTCCAGACGCGCCAGGG
GAGACAGTGAATTTGACATGTGACACACCAGAAGAAGATGACATTACATGGACATCTGACCAAC
GCCATGGCGTAATAGGGAGTGGGAAAACACTCACGATCACAGTTAAAGAGTTCTTGGATGCTGG
TCAATATACTTGCCATAAAGGCGGCGAGACACTCAGCCACTCACATTTGCTTTTGCATAAAAAAG
AGAATGGCATTTGGAGCACTGAAATACTTAAGAACTTTAAGAACAAGACATTTCTCAAGTGTGA
GGCCCCTAATTACAGCGGCAGGTTCACGTGCTCATGGCTGGTCCAGCGCAACATGGACCTCAAG
TTTAACATAAAATCTTCTTCCTCTTCACCTGACTCCAGAGCTGTTACTTGCGGCATGGCTTCTCTG
AGCGCAGAAAAAGTAACGTTGGATCAAAGAGACTACGAAAAGTACTCTGTTTCTTGTCAAGAGG
ATGTTACGTGCCCGACGGCCGAAGAAACGCTTCCAATTGAACTCGCGTTGGAAGCTCGCCAACA
AAACAAGTATGAAAACTACAGTACAAGCTTCTTTATACGGGATATAATTAAACCCGATCCCCCC
AAGAACTTGCAAATGAAACCACTTAAGAACAGCCAGGTGGAAGTTTCCTGGGAGTATCCAGACT
CATGGAGTACTCCTCACAGCTATTTTTCTCTGAAATTCTTTGTAAGGATACAACGGAAGAAAGAG
AAGATGAAAGAGACCGAGGAGGGTTGTAATCAGAAGGGAGCGTTTCTCGTGGAGAAAACGTCT
ACCGAAGTCCAATGTAAAGGTGGCAATGTGTGCGTCCAAGCTCAGGATAGATACTATAATTCAA
GTTGCTCCAAGTGGGCCTGTGTTCCATGCCGCGTTCGGAGCGGGGGAGGTAGCGGAGGAGGTAG
TGGGGGTGGGTCAGGAGGAGGGAGTCGAGTTATCCCGGTGTCAGGCCCCGCACGCTGCTTGAGC
CAGAGTCGCAACCTCCTTAAGACAACAGATGACATGGTGAAAACAGCACGCGAAAAGCTTAAA
CACTACTCTTGTACGGCGGAGGATATTGATCACGAGGATATTACCCGAGACCAAACTAGCACTTT
GAAAACCTGTCTGCCCCTTGAACTTCATAAAAATGAGAGCTGTCTGGCTACACGAGAGACGTCA
AGTACGACTAGGGGCAGCTGTCTCCCGCCGCAAAAGACAAGCCTCATGATGACGCTCTGTTTGG
GTTCCATTTACGAGGACTTGAAAATGTATCAAACGGAGTTCCAGGCTATAAATGCGGCGTTGCA
GAACCATAACCATCAACAAATTATACTTGATAAAGGCATGTTGGTGGCGATTGATGAACTCATG
CAGAGTCTCAATCACAACGGGGAAACGTTGAGACAGAAACCCCCAGTCGGTGAAGCGGACCCA
TATCGAGTAAAAATGAAGCTCTGCATTCTGCTTCACGCATTCAGCACTAGAGTTGTTACCATCAA
CCGGGTAATGGGATATCTCTCCAGTGCGCGGCGCAAGAGGGGTTCCGGAGAGGGAAGGGGTAG
TCTGCTCACCTGCGGCGATGTTGAAGAAAATCCTGGTCCCATGGCGCAAATGATGACCCTTTCCC
TGCTGAGTCTTGTCCTCGCGCTCTGCATCCCGTGGACGCAGGGGTCTGATGGGGGGGGCCAAGA
CTGTTGCCTGAAGTATTCACAAAAAAAGATACCGTACTCTATTGTCAGAGGGTACAGGAAGCAA
GAACCCTCCTTGGGTTGCCCTATACCAGCAATTCTTTTCTCCCCACGCAAGCATTCCAAACCAGA
ACTGTGTGCGAACCCCGAGGAGGGTTGGGTACAGAACTTGATGCGAAGGCTTGACCAGCCCCCA
GCCCCTGGCAAGCAGTCACCTGGGTGCAGAAAAAACAGAGGTACTTCAAAGAGCGGCAAGAAA
GGCAAAGGGAGTAAAGGATGTAAAAGAACGGAGCAGACCCAGCCTTCACGAGGCtaG
CCL21a_2A_IL-12 (Mouse) (SEQ ID NO: 63)
ATGGCGCAAATGATGACCCTTTCCCTGCTGAGTCTTGTCCTCGCGCTCTGCATCCCGTGGACGCA
GGGGTCTGATGGGGGGGGCCAAGACTGTTGCCTGAAGTATTCACAAAAAAAGATACCGTACTCT
ATTGTCAGAGGGTACAGGAAGCAAGAACCCTCCTTGGGTTGCCCTATACCAGCAATTCTTTTCTC
CCCACGCAAGCATTCCAAACCAGAACTGTGTGCGAACCCCGAGGAGGGTTGGGTACAGAACTTG
ATGCGAAGGCTTGACCAGCCCCCAGCCCCTGGCAAGCAGTCACCTGGGTGCAGAAAAAACAGA
GGTACTTCAAAGAGCGGCAAGAAAGGCAAAGGGAGTAAAGGATGTAAAAGAACGGAGCAGAC
CCAGCCTTCACGAGGCCGGCGCAAGAGGGGTTCCGGAGAGGGAAGGGGTAGTCTGCTCACCTGC
GGCGATGTTGAAGAAAATCCTGGTCCCATGTGTCCACAGAAGCTGACAATAAGTTGGTTTGCCA
TTGTCCTCCTGGTGAGCCCACTCATGGCAATGTGGGAACTCGAAAAGGATGTCTACGTGGTAGA
AGTAGATTGGACTCCAGACGCGCCAGGGGAGACAGTGAATTTGACATGTGACACACCAGAAGA
AGATGACATTACATGGACATCTGACCAACGCCATGGCGTAATAGGGAGTGGGAAAACACTCACG
ATCACAGTTAAAGAGTTCTTGGATGCTGGTCAATATACTTGCCATAAAGGCGGCGAGACACTCA
GCCACTCACATTTGCTTTTGCATAAAAAAGAGAATGGCATTTGGAGCACTGAAATACTTAAGAA
CTTTAAGAACAAGACATTTCTCAAGTGTGAGGCCCCTAATTACAGCGGCAGGTTCACGTGCTCAT
GGCTGGTCCAGCGCAACATGGACCTCAAGTTTAACATAAAATCTTCTTCCTCTTCACCTGACTCC
AGAGCTGTTACTTGCGGCATGGCTTCTCTGAGCGCAGAAAAAGTAACGTTGGATCAAAGAGACT
ACGAAAAGTACTCTGTTTCTTGTCAAGAGGATGTTACGTGCCCGACGGCCGAAGAAACGCTTCC
AATTGAACTCGCGTTGGAAGCTCGCCAACAAAACAAGTATGAAAACTACAGTACAAGCTTCTTT
ATACGGGATATAATTAAACCCGATCCCCCCAAGAACTTGCAAATGAAACCACTTAAGAACAGCC
AGGTGGAAGTTTCCTGGGAGTATCCAGACTCATGGAGTACTCCTCACAGCTATTTTTCTCTGAAA
TTCTTTGTAAGGATACAACGGAAGAAAGAGAAGATGAAAGAGACCGAGGAGGGTTGTAATCAG
AAGGGAGCGTTTCTCGTGGAGAAAACGTCTACCGAAGTCCAATGTAAAGGTGGCAATGTGTGCG
TCCAAGCTCAGGATAGATACTATAATTCAAGTTGCTCCAAGTGGGCCTGTGTTCCATGCCGCGTT
CGGAGCGGGGGAGGTAGCGGAGGAGGTAGTGGGGGTGGGTCAGGAGGAGGGAGTCGAGTTATC
CCGGTGTCAGGCCCCGCACGCTGCTTGAGCCAGAGTCGCAACCTCCTTAAGACAACAGATGACA
TGGTGAAAACAGCACGCGAAAAGCTTAAACACTACTCTTGTACGGCGGAGGATATTGATCACGA
GGATATTACCCGAGACCAAACTAGCACTTTGAAAACCTGTCTGCCCCTTGAACTTCATAAAAATG
AGAGCTGTCTGGCTACACGAGAGACGTCAAGTACGACTAGGGGCAGCTGTCTCCCGCCGCAAAA
GACAAGCCTCATGATGACGCTCTGTTTGGGTTCCATTTACGAGGACTTGAAAATGTATCAAACGG
AGTTCCAGGCTATAAATGCGGCGTTGCAGAACCATAACCATCAACAAATTATACTTGATAAAGG
CATGTTGGTGGCGATTGATGAACTCATGCAGAGTCTCAATCACAACGGGGAAACGTTGAGACAG
AAACCCCCAGTCGGTGAAGCGGACCCATATCGAGTAAAAATGAAGCTCTGCATTCTGCTTCACG
CATTCAGCACTAGAGTTGTTACCATCAACCGGGTAATGGGATATCTCTCCAGTGCGtaG
IL7 (Mouse) (SEQ ID NO: 64)
ATGTTTCATGTGTCCTTCAGGTACATATTTGGTATCCCACCACTTATATTGGTGCTCTTGCCTGTA
ACCAGCTCTGAATGTCATATAAAAGACAAGGAGGGCAAAGCATACGAGTCCGTATTGATGATCT
CAATCGATGAACTTGACAAGATGACAGGGACCGATTCTAATTGTCCAAATAACGAGCCAAACTT
CTTTCGGAAACACGTGTGTGATGATACAAAAGAAGCTGCTTTTCTTAACAGAGCTGCCAGAAAA
CTCAAGCAGTTCCTCAAGATGAATATATCCGAGGAATTTAACGTGCATCTCCTCACAGTATCTCA
GGGAACTCAAACCCTTGTAAACTGCACTTCTAAGGAGGAGAAGAATGTCAAAGAGCAGAAGAA
AAATGATGCATGTTTTTTGAAACGGCTGTTGAGGGAGATCAAAACATGCTGGAATAAAATCCTC
AAGGGCTCAATTtaG
IL-15 (Human) (SEQ ID NO: 65)
ATGGAAACAGACACATTGCTGCTTTGGGTATTGTTGCTCTGGGTGCCTGGATCAACAGGAAACTG
GGTAAACGTAATTTCAGATCTGAAGAAGATCGAGGACCTTATTCAATCCATGCACATCGATGCC
ACTCTCTACACCGAAAGCGACGTTCACCCATCTTGCAAGGTGACCGCTATGAAATGTGAATTGTT
GGAACTTCAGGTAATTTCTCTGGAGAGCGGCGATGCCTCAATACATGACACCGTTGAAAATCTTA
TCATCCTTGCTAATGATTCACTCTCTAGTAATGGGAACGTAACAGAGAGCGGGTGTAAGGAGTG
TGAAGAACTGGAGGAGAAAAACATTAAGGAATTTTTGCAGTCATTCGTCCATATAGTGCAAATG
TTCATAAACACTTCCAGAAGAAAGCGAGGCTCTGGGGAGGGGCGAGGCTCTCTGCTGACCTGTG
GGGATGTAGAAGAGAATCCAGGTCCCATGGACCGGCTGACCAGCTCATTCCTGCTTCTGATTGTG
CCAGCCTACGTGCTCTCCATCACATGTCCTCCCCCAATGAGCGTCGAGCATGCTGACATCTGGGT
GAAGTCATACTCCTTGTACAGCAGAGAGAGATACATTTGTAATTCCGGATTCAAGCGCAAGGCC
GGCACCTCCTCTCTGACAGAGTGCGTCCTTAACAAAGCAACCAACGTAGCACATTGGACCACAC
CATCCTTGAAGTGCATACGAGAACCTAAATCTTGCGATAAGACTCATACTTGTCCACCTTGTCCA
GCCCCAGAACTGCTTGGCGGACCCTCAGTATTTTTGTTCCCACCAAAGCCAAAAGACACACTCAT
GATATCCAGAACTCCTGAGGTGACCTGTGTCGTTGTAGACGTTTCCCACGAAGATCCTGAAGTAA
AATTCAACTGGTACGTGGATGGGGTCGAAGTCCATAACGCCAAGACTAAACCAAGGGAGGAAC
AGTATAACTCTACTTACCGAGTAGTTTCTGTGTTGACCGTGCTGCACCAGGACTGGTTGAACGGG
AAGGAGTACAAATGCAAGGTGAGCAATAAAGCTCTGCCCGCACCAATCGAAAAGACAATATCT
AAGGCCAAGGGGCAGCCACGAGAGCCCCAGGTATACACACTGCCACCCTCACGCGATGAATTG
ACTAAGAACCAGGTTTCCCTGACCTGTCTTGTAAAAGGTTTCTACCCTTCCGACATAGCTGTTGA
GTGGGAAAGTAACGGGCAGCCAGAGAACAATTACAAGACAACTCCACCCGTTCTTGATAGCGAT
GGATCATTTTTTCTGTATTCCAAACTCACTGTCGATAAAAGTCGCTGGCAGCAAGGCAATGTTTT
TAGCTGCTCAGTCATGCACGAAGCACTGCATAATCACTACACACAAAAAAGTTTGTCCCTTAGCC
CTGGTAAGtaG
IL-15 (Human) (SEQ ID NO: 66)
ATGTACTCAATGCAGTTGGCCTCCTGTGTAACATTGACCTTGGTCCTCTTGGTCAACAGCAATTG
GATCGATGTACGCTACGACTTGGAGAAGATTGAGTCCCTTATACAGAGTATACACATAGATACA
ACCTTGTATACTGACAGTGACTTCCATCCCAGCTGTAAAGTGACTGCAATGAACTGTTTTTTGTT
GGAGTTGCAAGTAATTCTGCATGAATACAGCAACATGACCCTCAATGAAACCGTTAGGAATGTC
CTTTATCTCGCAAATTCTACTCTGAGTAGCAATAAGAATGTTGCCGAAAGCGGCTGCAAGGAGT
GCGAAGAACTGGAGGAAAAAACTTTCACCGAGTTTCTCCAGAGTTTCATCAGAATTGTCCAAAT
GTTCATTAATACAAGTAGTGGTGGTGGGAGCGGGGGTGGAGGCAGTGGGGGAGGTGGGAGCGG
AGGTGGAGGGTCCGGAGGGGGGAGCCTTCAAGGCACTACTTGTCCTCCACCCGTATCCATCGAG
CACGCCGATATTCGAGTTAAAAATTATAGTGTTAATAGCAGAGAACGATACGTCTGCAACTCAG
GGTTTAAGAGAAAGGCCGGAACTTCAACTCTCATAGAATGCGTGATTAATAAGAATACTAACGT
CGCACATTGGACTACTCCCAGTCTCAAGTGCATACGCGATCCATCTCTCGCTCATTACTCACCAG
TACCTACAGTGGTTACTCCTAAGGTGACCTCTCAGCCCGAATCACCATCTCCCAGCGCAAAAGAG
CCTGAGGCCTTTTCTCCTAAATCAGACACTGCTATGACTACAGAAACAGCCATAATGCCAGGAA
GCCGGCTGACACCATCTCAAACTACCAGCGCAGGCACAACTGGGACTGGCTCCCACAAAAGCTC
ACGCGCACCAAGTCTCGCCGCAACAATGACATTGGAGCCTACAGCCAGCACATCTCTTAGAATC
ACAGAAATTTCTCCCCACAGTAGCAAGATGACCAAGGTGGCAATTAGTACCAGCGTCCTTCTTGT
AGGAGCTGGAGTTGTGATGGCATTTTTGGCATGGTATATCAAAAGCAGGtaG
IL-15 (Mouse) (SEQ ID NO: 67)
ATGAAGATCCTCAAGCCATACATGCGAAACACTAGTATTAGCTGTTACTTGTGTTTTCTGCTGAA
TAGTCATTTTTTGACTGAAGCAGGAATCCATGTATTTATACTCGGTTGTGTGTCTGTAGGTCTGCC
AAAGACTGAGGCTAATTGGATTGACGTGCGCTATGATCTTGAAAAAATAGAGTCCTTGATTCAA
TCAATACACATCGATACCACTCTCTACACCGACAGTGATTTCCATCCTTCCTGCAAGGTAACAGC
TATGAATTGCTTCCTCCTGGAGCTCCAAGTCATTCTCCATGAGTACTCCAACATGACTTTGAACG
AAACTGTAAGAAACGTATTGTATCTGGCTAATAGCACCTTGTCTAGTAACAAAAATGTGGCAGA
GAGCGGCTGCAAAGAATGTGAAGAATTGGAAGAGAAAACATTTACAGAGTTCCTGCAATCCTTT
ATTCGCATCGTCCAAATGTTTATCAATACCTCTtaG
IL-15 (Mouse) (SEQ ID NO: 68)
ATGTATTCCATGCAACTTGCCAGTTGTGTAACCCTTACTCTCGTCCTGCTCGTTAATTCCGCTGGT
GCTAACTGGATAGATGTTCGATACGATCTGGAAAAGATTGAGTCCCTTATCCAATCCATTCATAT
AGATACCACCCTTTATACTGACAGCGACTTCCATCCTTCTTGCAAGGTGACCGCTATGAATTGTT
TCCTGCTGGAACTCCAAGTTATTCTGCATGAATACTCTAATATGACACTTAACGAGACCGTAAGA
AATGTTCTCTATCTCGCTAATAGTACTTTGAGCTCAAATAAGAACGTGGCCGAGTCTGGGTGTAA
GGAATGCGAAGAGCTGGAAGAAAAGACATTCACCGAGTTTCTCCAGTCTTTCATACGGATTGTG
CAGATGTTTATCAACACATCAGATTACAAAGACGACGATGATAAGtaG
IL-18 (Mouse) (SEQ ID NO: 69)
ATGGCAGCCATGTCTGAGGACTCTTGTGTGAACTTTAAAGAAATGATGTTCATAGACAATACACT
CTACTTTATACCTGAGGAGAATGGAGATTTGGAATCTGACAACTTTGGCAGGCTGCATTGCACTA
CCGCAGTTATCCGAAACATCAACGATCAGGTACTGTTTGTTGATAAAAGACAACCTGTATTCGAG
GACATGACCGACATAGATCAGTCTGCCTCAGAGCCCCAGACTAGGCTTATCATCTATATGTACAA
GGACAGCGAAGTACGAGGCCTGGCTGTTACACTCTCAGTCAAAGACTCTAAGATGAGCACCCTG
TCATGCAAGAACAAAATTATCAGTTTTGAGGAGATGGACCCACCTGAAAACATAGATGACATTC
AGTCAGACCTCATTTTTTTTCAAAAGCGGGTACCAGGACACAACAAAATGGAATTTGAATCATC
ACTCTACGAAGGACATTTCCTTGCATGCCAGAAAGAGGATGACGCATTCAAATTGATCCTGAAA
AAAAAGGACGAAAATGGTGATAAATCAGTCATGTTTACATTGACCAATCTTCACCAAAGTtaG
IL-18 (Mouse) (SEQ ID NO: 70)
ATGGCTGCAATGTCTGAAGATAGCTGTGTCAACTTTAAGGAGATGATGTTCATTGATAATACTTT
GTACTTTATACCTGAAGAAAATGGAGACCTTGAGTCAGACAACTTCGGGAGACTGCACTGCACA
ACTGCCGTTATCCGAAACATAAATGATCAAGTATTGTTCGTGGACAAAAGACAACCAGTCTTTG
AGGATATGACAGACATCGACCAATCCGCATCTGAACCTCAGACTAGGCTGATCATCTATATGTA
CGCCGACTCCGAAGTAAGAGGCCTTGCTGTGACACTTAGTGTTAAGGATAGTAAGATGAGCACA
CTGTCCTGTAAGAATAAGATTATATCTTTTGAAGAGATGGACCCTCCCGAGAACATAGATGACAT
CCAGAGCGACTTGATCTTCTTTCAGAAGCGAGTGCCAGGCCATAACAAGATGGAATTTGAATCA
TCTCTTTATGAAGGCCATTTCCTCGCATGTCAAAAGGAGGACGATGCCTTCAAGCTCATTCTGAA
AAAAAAAGACGAGAACGGTGATAAGAGCGTGATGTTCACTCTGACAAATCTGCACCAGTCAtaG
IL-18 (Human) (SEQ ID NO: 71)
ATGTATCGCATGCAACTCCTGTCCTGCATTGCTCTGAGCTTGGCTTTGGTAACCAACTCATACTTC
GGGAAACTGGAGAGTAAACTCTCCGTAATCAGGAATCTTAATGACCAAGTATTGTTTATTGACC
AGGGCAACCGCCCGTTGTTCGAGGATATGACTGATTCTGACTGTCGGGATAACGCTCCGAGAAC
TATCTTTATCATTTCAATGTACAAGGACAGCCAACCGCGGGGTATGGCTGTGACAATCAGTGTCA
AATGTGAGAAGATTTCCACGCTGTCCTGCGAAAACAAGATAATTTCTTTCAAAGAAATGAACCC
CCCTGACAATATAAAGGATACAAAGAGTGATATCATCTTCTTTCAGAGGTCCGTGCCCGGCCAC
GATAATAAGATGCAATTTGAAAGTTCATCTTATGAGGGGTACTTTTTGGCATGCGAGAAAGAAA
GGGATCTCTTCAAGTTGATCCTGAAGAAGGAGGACGAATTGGGCGACCGCTCCATCATGTTCAC
AGTCCAGAACGAGGACtaG
IL-18 (Human) (SEQ ID NO: 72)
ATGTACCGCATGCAGCTCCTGAGTTGTATTGCCCTTTCCCTCGCTCTCGTTACCAATTCTTACTTC
GGTAAGCTTGCCTCTAAACTCTCTGTTATTAGGAACTTGAACGACCAAGTCCTTTTCATAGACCA
AGGGAACAGACCACTGTTTGAAGATATGACGGATAGCGATTGCCGAGATAATGCCCCTAGGACG
ATTTTTATCATTAGTATGTATGCGGACTCTCAACCGAGGGGGATGGCCGTTACTATAAGTGTGAA
ATGCGAGAAAATATCAACGCTCAGTTGTGAGAACAAAATCATAAGTTTCAAGGAGATGAATCCA
CCTGATAACATCAAAGACACTAAGTCTGATATTATATTTTTCCAACGAAGTGTTCCGGGACACGA
TAACAAAATGCAATTTGAGAGCTCCTCATACGAGGGCTACTTCCTCGCGTGTGAGAAAGAAAGG
GATTTGTTTAAGCTTATCCTCAAGAAAGAGGACGAGTTGGGGGATCGGAGCATAATGTTTACCG
TACAGAATGAGGACtaG
IL-21 (Mouse) (SEQ ID NO: 73)
ATGGAGCGGACACTCGTGTGTCTTGTCGTAATTTTTCTCGGGACAGTCGCACACAAGTCCTCACC
CCAGGGTCCTGATCGCCTTCTCATACGCCTCCGACATTTGATCGACATTGTAGAGCAGCTCAAAA
TTTACGAGAATGACCTCGATCCCGAGCTTTTGAGTGCTCCCCAAGACGTTAAGGGTCATTGCGAG
CACGCAGCTTTTGCTTGCTTCCAGAAGGCCAAGTTGAAACCAAGCAACCCTGGTAATAATAAGA
CTTTCATCATCGACTTGGTCGCCCAACTCCGAAGGAGGCTGCCTGCCCGGCGCGGAGGAAAAAA
ACAAAAGCATATTGCAAAGTGTCCTTCATGTGATTCATACGAAAAGCGGACTCCCAAAGAGTTC
TTGGAAAGGTTGAAATGGCTTCTTCAGAAGATGATTCATCAACATTTGTCAtaG
IFN-beta (Human) (SEQ ID NO: 74)
ATGACCAACAAATGCCTTTTGCAAATTGCCCTGCTTTTGTGTTTTAGCACTACCGCATTGAGCAT
GTCATATAACCTCCTCGGCTTCCTTCAGAGATCATCAAACTTTCAGTGTCAGAAACTGCTTTGGC
AACTTAATGGCAGGCTCGAATATTGTCTGAAAGATCGGATGAATTTCGACATTCCAGAAGAAAT
AAAACAGCTTCAACAATTCCAGAAAGAGGACGCCGCCCTGACTATTTACGAGATGCTCCAGAAT
ATCTTCGCCATTTTCCGGCAGGACAGCTCATCCACGGGGTGGAATGAGACTATTGTAGAAAATCT
TCTGGCTAATGTGTACCATCAAATTAATCACCTCAAAACGGTGCTTGAGGAAAAACTTGAAAAG
GAAGATTTCACACGGGGCAAGTTGATGTCCTCCCTGCACCTTAAACGATACTACGGCAGGATTCT
TCATTACTTGAAGGCTAAGGAGTATAGCCATTGCGCGTGGACAATTGTACGGGTAGAAATACTG
CGAAACTTTTATTTCATCAACCGGCTCACTGGATACCTTAGAAATtaG
IFN-beta (Mouse) (SEQ ID NO: 75)
ATGAACAATCGGTGGATACTCCACGCCGCATTTCTCCTCTGCTTTAGCACGACGGCCCTGTCCAT
CAACTACAAACAGCTTCAGTTGCAGGAGCGGACTAACATAAGGAAGTGCCAGGAACTGCTGGA
ACAGCTTAATGGTAAAATTAATCTTACATACCGAGCTGACTTCAAAATTCCTATGGAAATGACCG
AGAAGATGCAGAAATCCTACACGGCATTCGCCATCCAGGAAATGCTCCAGAACGTATTTCTCGT
GTTCCGCAATAATTTCTCTTCTACGGGTTGGAACGAAACCATTGTTGTTAGACTGCTTGACGAAC
TGCATCAGCAAACCGTGTTCCTTAAAACCGTGCTTGAGGAGAAGCAGGAGGAGCGCCTGACTTG
GGAGATGTCTAGTACCGCACTTCACTTGAAATCCTACTACTGGCGCGTTCAGCGGTATCTGAAGC
TGATGAAGTATAACTCATACGCCTGGATGGTAGTGCGCGCAGAGATCTTCAGAAACTTTCTTATC
ATCCGGCGACTGACCCGAAACTTTCAGAATtaG
IFN-gamma (Human) (SEQ ID NO: 76)
ATGAAGTACACTAGCTATATATTGGCCTTCCAGCTTTGCATCGTATTGGGTAGCCTCGGATGCTA
TTGCCAAGACCCGTATGTCAAAGAAGCCGAAAATCTCAAAAAGTATTTCAATGCCGGACACTCA
GACGTCGCGGATAACGGTACACTGTTTCTTGGCATCCTGAAAAATTGGAAGGAAGAGAGTGACA
GAAAAATAATGCAGTCACAAATAGTGTCCTTTTACTTTAAGCTGTTCAAAAATTTCAAGGATGAC
CAAAGTATCCAGAAGAGTGTTGAAACTATCAAAGAGGACATGAATGTGAAATTCTTTAACAGTA
ATAAGAAGAAGCGCGATGACTTCGAGAAACTCACTAATTACAGCGTAACGGATCTTAACGTCCA
ACGCAAGGCAATCCACGAGCTTATACAGGTAATGGCTGAGCTTAGTCCCGCAGCCAAGACAGGG
AAGAGAAAAAGGTCTCAAATGCTTTTTCGGGGCCGGCGAGCTTCACAAtaG
IFN-gamma (Mouse) (SEQ ID NO: 77)
ATGAACGCTACGCATTGCATCCTCGCACTCCAATTGTTCCTCATGGCTGTGTCAGGGTGTTACTG
TCACGGTACTGTCATAGAAAGCCTCGAATCCCTGAATAACTATTTTAACAGTAGCGGTATAGATG
TAGAAGAAAAGTCTCTCTTTCTTGACATCTGGAGGAATTGGCAAAAGGATGGAGACATGAAGAT
TCTCCAATCTCAGATTATATCATTTTACTTGAGGCTTTTTGAGGTTCTGAAGGATAACCAGGCGA
TCAGCAATAATATCAGCGTAATTGAATCTCACCTTATTACAACATTTTTCTCAAATTCCAAGGCA
AAGAAAGATGCTTTCATGTCTATCGCGAAATTTGAGGTGAACAATCCTCAGGTACAAAGGCAAG
CCTTTAACGAGCTGATTAGAGTTGTACATCAGTTGTTGCCCGAAAGTAGTCTTAGAAAACGCAAA
CGGAGCCGATGCtaG
IFN-alpha (Mouse) (SEQ ID NO: 78)
ATGGCAAGGTTGTGCGCTTTTCTCATGGTACTGGCTGTGCTCTCCTATTGGCCTACTTGTTCTCTG
GGATGCGACTTGCCACAGACCCACAATCTGCGGAATAAGAGGGCTCTGACTCTGCTGGTGCAAA
TGAGACGGCTCTCTCCACTTAGCTGTTTGAAAGATAGAAAGGATTTCGGGTTCCCCCAGGAGAA
GGTGGATGCCCAGCAGATCAAGAAGGCACAGGCTATCCCCGTCCTTTCCGAGCTGACCCAGCAA
ATTTTGAACATCTTTACAAGTAAGGATAGTTCAGCTGCATGGAATACCACACTTTTGGATTCTTTT
TGTAACGATCTGCATCAGCAGCTGAACGATCTCCAGGGATGCCTGATGCAGCAAGTCGGCGTGC
AAGAATTTCCACTCACCCAGGAGGACGCTCTGCTCGCAGTGCGAAAGTATTTTCACCGAATTACC
GTGTACCTCCGGGAGAAAAAGCATTCACCCTGCGCTTGGGAAGTAGTCAGGGCCGAAGTATGGA
GAGCCCTTAGTAGCTCCGCTAATGTACTGGGCCGGTTGCGGGAAGAGAAAtaG
CCL21 (Human) (SEQ ID NO: 79)
ATGGCGCAAAGTCTGGCTCTTTCACTCCTGATCCTGGTCTTGGCCTTCGGGATTCCGAGGACCCA
AGGAAGTGATGGTGGCGCCCAAGATTGTTGCCTTAAATACAGCCAGCGGAAAATACCCGCGAAA
GTGGTCAGGAGTTATAGAAAACAGGAGCCTTCCCTGGGTTGTAGTATCCCCGCCATACTTTTCCT
CCCGAGAAAACGGAGCCAGGCCGAACTGTGCGCTGACCCTAAGGAACTTTGGGTGCAACAACTT
ATGCAACACCTGGATAAGACACCTTCTCCTCAAAAGCCAGCTCAGGGCTGCCGAAAAGATAGAG
GCGCCTCAAAAACCGGAAAAAAGGGCAAAGGTTCTAAAGGATGTAAGCGGACTGAACGCTCTC
AAACGCCTAAAGGGCCGtaG
CCL21a (Mouse) (SEQ ID NO: 80)
ATGGCGCAAATGATGACCCTTTCCCTGCTGAGTCTTGTCCTCGCGCTCTGCATCCCGTGGACGCA
GGGGTCTGATGGGGGGGGCCAAGACTGTTGCCTGAAGTATTCACAAAAAAAGATACCGTACTCT
ATTGTCAGAGGGTACAGGAAGCAAGAACCCTCCTTGGGTTGCCCTATACCAGCAATTCTTTTCTC
CCCACGCAAGCATTCCAAACCAGAACTGTGTGCGAACCCCGAGGAGGGTTGGGTACAGAACTTG
ATGCGAAGGCTTGACCAGCCCCCAGCCCCTGGCAAGCAGTCACCTGGGTGCAGAAAAAACAGA
GGTACTTCAAAGAGCGGCAAGAAAGGCAAAGGGAGTAAAGGATGTAAAAGAACGGAGCAGAC
CCAGCCTTCACGAGGCtaG
Tail-less CCL21 (Human) (SEQ ID NO: 81)
ATGGCGCAAAGTCTGGCTCTTTCACTCCTGATCCTGGTCTTGGCCTTCGGGATTCCGAGGACCCA
AGGAAGTGATGGTGGCGCCCAAGATTGTTGCCTTAAATACAGCCAGCGGAAAATACCCGCGAAA
GTGGTCAGGAGTTATAGAAAACAGGAGCCTTCCCTGGGTTGTAGTATCCCCGCCATACTTTTCCT
CCCGAGAAAACGGAGCCAGGCCGAACTGTGCGCTGACCCTAAGGAACTTTGGGTGCAACAACTT
ATGCAACACCTGGATAAGACACCTTCTCCTCAAAAGCCAGCTCAGGGCtaG
Tail-less CCL21 (Mouse) (SEQ ID NO: 82)
ATGGCGCAAATGATGACCCTTTCCCTGCTGAGTCTTGTCCTCGCGCTCTGCATCCCGTGGACGCA
GGGGTCTGATGGGGGGGGCCAAGACTGTTGCCTGAAGTATTCACAAAAAAAGATACCGTACTCT
ATTGTCAGAGGGTACAGGAAGCAAGAACCCTCCTTGGGTTGCCCTATACCAGCAATTCTTTTCTC
CCCACGCAAGCATTCCAAACCAGAACTGTGTGCGAACCCCGAGGAGGGTTGGGTACAGAACTTG
ATGCGAAGGCTTGACCAGCCCCCAGCCCCTGGCAAGCAGTCACCTGGGtaG
CCL19 (Mouse) (SEQ ID NO: 83)
ATGGCACCCCGCGTCACACCCTTGCTTGCTTTTTCTCTGCTTGTCCTCTGGACCTTCCCCGCTCCT
ACCCTTGGAGGAGCCAATGATGCCGAGGATTGCTGCCTGAGTGTTACACAAAGGCCAATACCAG
GGAATATAGTGAAGGCATTCCGGTATCTGCTCAATGAAGATGGGTGCAGAGTCCCCGCAGTTGT
CTTTACAACATTGCGAGGTTACCAGCTTTGTGCTCCCCCAGACCAGCCTTGGGTAGATCGCATTA
TTCGCCGGTTGAAGAAGAGCTCAGCAAAGAATAAGGGCAATTCCACACGGAGAAGCCCCGTCTC
CtaG
CCL19 (Mouse) (SEQ ID NO: 84)
ATGAAATCAGCAGTCCTTTTCTTGCTCGGGATTATTTTTCTGGAACAATGTGGAGTGAGGGGAAC
ACTCGTAATAAGAAACGCTCGGTGCTCATGCATATCAACATCACGGGGCACTATCCACTACAAA
TCCCTGAAGGATCTGAAGCAGTTCGCCCCAAGCCCTAACTGTAACAAGACCGAAATTATCGCAA
CTCTCAAAAATGGAGATCAGACTTGTCTTGACCCAGATTCAGCAAATGTCAAGAAGCTGATGAA
AGAGTGGGAAAAGAAGATTTCACAAAAAAAAAAGCAAAAACGCGGCAAGAAACATCAAAAGA
ACATGAAAAACAGGAAACCTAAGACTCCCCAGTCAAGGAGAAGATCCCGCAAGACAACCtaG
CXCL11 (Mouse) (SEQ ID NO: 85)
ATGAACAGAAAAGTTACCGCTATAGCACTTGCTGCCATAATATGGGCCACCGCAGCTCAAGGGT
TCCTGATGTTCAAGCAGGGCCGATGCCTCTGCATTGGCCCTGGAATGAAGGCCGTGAAAATGGC
CGAAATAGAAAAAGCTAGTGTCATATACCCCTCTAACGGTTGCGATAAAGTCGAGGTTATAGTC
ACAATGAAAGCTCATAAACGCCAACGCTGCCTCGACCCCCGGTCTAAGCAGGCTAGGCTCATAA
TGCAAGCAATCGAGAAGAAAAACTTTCTTAGACGGCAAAACATGtaG
CXCL10 (Mouse) (SEQ ID NO: 86)
ATGAACCCATCTGCCGCCGTTATTTTCTGTCTGATACTCCTTGGGCTGAGTGGCACACAAGGCAT
ACCCCTCGCCCGCACAGTCCGGTGTAATTGTATACATATTGACGACGGCCCTGTTAGAATGCGGG
CCATCGGTAAGCTGGAGATTATACCAGCAAGCCTTAGTTGTCCCAGGGTTGAAATCATAGCAAC
TATGAAAAAAAACGACGAACAAAGATGTTTGAATCCCGAGAGCAAGACAATCAAAAACCTTAT
GAAAGCATTTAGTCAAAAACGCTCTAAACGCGCTCCAtaG
CXCL10 (Human) (SEQ ID NO: 87)
ATGAATCAGACGGCAATCCTTATATGCTGCCTTATATTCCTTACTCTCTCAGGGATACAAGGGGT
ACCACTTTCTCGGACTGTTCGCTGCACTTGCATTTCAATATCTAACCAACCTGTAAATCCGCGGA
GCCTGGAAAAATTGGAGATTATACCTGCTTCTCAATTCTGCCCTCGGGTGGAAATCATCGCCACT
ATGAAGAAGAAGGGCGAGAAAAGGTGTCTGAATCCAGAGTCAAAGGCAATCAAAAACCTGCTG
AAAGCGGTGTCAAAGGAACGGTCCAAGAGATCACCCtaG
CXCL11-CXCL10 (Mouse) (SEQ ID NO: 88)
ATGAACAGGAAAGTAACAGCCATTGCATTGGCTGCCATCATCTGGGCCACCGCAGCACAGGGTT
TTCTGATGTTTAAGCAAGGGCGCTGTCTCTGTATAGGCCCAGGCATGAAGGCCGTGAAGATGGC
AGAGATTGAGAAGGCATCTGTGATTTATCCTTCTAACGGGTGCGATAAAGTCGAAGTTATTGTGA
CAATGAAGGCACACAAACGCCAACGGTGTTTGGACCCACGATCTAAACAGGCAAGATTGATTAT
GCAAGCCATCGAGAAAAAGAACTTTCTCCGAAGGCAAAATATGATCCCTTTGGCTCGGACAGTG
CGGTGTAACTGTATTCACATCGACGATGGGCCAGTACGGATGAGAGCAATAGGAAAGCTCGAAA
TCATACCCGCCTCATTGTCTTGTCCCAGGGTGGAAATAATCGCCACTATGAAAAAGAACGATGA
ACAGAGGTGTCTCAACCCAGAGAGTAAGACTATCAAGAACCTTATGAAGGCATTCAGTCAGAAG
AGGTCAAAGCGAGCACCAtaG
XCL1 (Human) (SEQ ID NO: 89)
ATGAGACTTCTCATATTGGCGCTTCTCGGGATATGTTCTCTTACGGCATACATAGTTGAGGGGGT
GGGATCTGAGGTTAGCGATAAACGAACTTGTGTTAGTCTTACAACACAGAGGCTTCCAGTCTCCA
GGATAAAAACATATACGATAACTGAGGGATCTCTCAGAGCGGTCATCTTCATAACGAAGAGGGG
CCTGAAGGTCTGTGCTGACCCACAAGCGACTTGGGTAAGGGACGTTGTGCGGAGCATGGACAGG
AAGAGCAATACTCGCAACAACATGATCCAAACCAAACCTACGGGCACCCAACAGTCAACCAAT
ACTGCGGTAACATTGACGGGGtaG
XCL1 (Mouse) (SEQ ID NO: 90)
ATGCGCCTCCTTCTGCTGACTTTTCTGGGTGTATGTTGCCTGACACCCTGGGTCGTAGAAGGAGT
AGGAACCGAGGTTCTGGAAGAGTCCTCATGTGTAAACTTGCAGACACAACGACTCCCCGTCCAA
AAAATCAAGACCTATATAATCTGGGAGGGGGCAATGCGGGCCGTCATTTTCGTGACTAAACGAG
GTCTCAAAATCTGCGCCGACCCCGAGGCTAAGTGGGTGAAGGCAGCCATTAAGACCGTGGATGG
GAGAGCCAGCACCAGAAAGAACATGGCCGAAACAGTACCTACTGGCGCACAGCGGTCAACCTC
AACTGCTATAACCTTGACAGGAtaG
m_sCD40L #1 (SEQ ID NO: 91)
ATGGAGACTGACACTCTGCTTCTGTGGGTGTTGCTGCTGTGGGTGCCTGGCAGTACAGGCGATAT
GCAACGAGGTGACGAGGACCCTCAAATCGCCGCCCATGTAGTCTCTGAAGCTAATAGCAACGCT
GCATCCGTCTTGCAGTGGGCAAAGAAAGGCTACTATACTATGAAGTCCAACTTGGTAATGCTTG
AAAACGGCAAGCAGTTGACTGTCAAGAGAGAGGGACTTTATTACGTCTATACCCAAGTCACATT
CTGTAGCAATCGAGAACCCTCCTCACAGAGGCCTTTTATAGTGGGACTCTGGCTTAAACCAAGTA
GCGGCTCTGAGCGCATACTGTTGAAAGCCGCAAACACACACAGCTCTTCCCAACTCTGCGAGCA
GCAATCCGTGCATCTCGGTGGAGTATTTGAGCTTCAAGCCGGTGCCTCAGTGTTTGTGAACGTCA
CTGAGGCCTCCCAGGTCATACATCGAGTTGGGTTCAGCTCCTTCGGCTTGCTCAAGCTCtaG
m_sCD40L #2 (SEQ ID NO: 92)
ATGGAAACTGATACATTGCTGCTCTGGGTTTTGCTGCTCTGGGTGCCTGGGAGTACAGGCGACAT
GAGGAGGCAGTTCGAGGATCTCGTTAAGGATATTACCCTTAATAAGGAGGAGAAGAAAGAAAA
CTCTTTTGAGATGCAACGAGGGGACGAAGATCCTCAGATCGCTGCTCACGTGGTCTCTGAAGCTA
ACAGCAACGCCGCTTCTGTCCTCCAGTGGGCCAAGAAAGGTTATTACACCATGAAATCAAACCT
TGTAATGCTTGAAAACGGGAAACAGCTTACAGTGAAGAGGGAAGGTCTTTACTACGTCTATACC
CAGGTAACCTTCTGCTCAAACAGAGAACCATCAAGCCAGAGGCCATTCATAGTGGGGCTCTGGC
TCAAACCTTCCAGTGGCAGCGAGAGAATCTTGTTGAAAGCTGCTAATACACATAGTAGTAGCCA
GCTTTGCGAGCAACAGTCAGTCCACCTCGGGGGGGTGTTTGAGTTGCAAGCAGGGGCCTCAGTA
TTCGTGAATGTCACTGAGGCTTCCCAGGTAATTCACAGGGTAGGCTTTAGTTCATTCGGTTTGCT
GAAGCTTtaG
m_sCD40L #3 (SEQ ID NO: 93)
ATGCGAAGAATGCAGCTTCTGCTCCTTATTGCTCTGAGTCTCGCCCTTGTCACCAACTCCGGGGA
CAGAATGAAACAAATCGAGGACAAAATTGAAGAAATACTGAGTAAAATATATCACATCGAAAA
CGAAATTGCACGCATTAAGAAATTGATTGGCGAACGCACCAGTGGCGGCTCTGGTGGCACCGGA
GGTTCAGGCGGGACCGGGGGCTCTGACAAAGTCGAAGAGGAGGTTAACCTTCATGAGGACTTTG
TGTTCATCAAGAAGCTGAAACGGTGCAATAAAGGAGAAGGTTCTTTGAGCCTCCTTAATTGCGA
AGAGATGCGACGACAGTTCGAGGATCTGGTTAAGGACATTACACTTAATAAGGAAGAGAAAAA
GGAGAACTCTTTCGAAATGCAGCGCGGCGATGAAGATCCCCAGATAGCCGCCCATGTCGTCTCT
GAGGCCAACTCTAACGCAGCATCCGTCCTCCAGTGGGCTAAGAAAGGATATTATACTATGAAAA
GCAATTTGGTCATGCTCGAAAACGGTAAACAGCTCACTGTTAAGAGAGAAGGCCTCTATTACGT
ATATACTCAAGTAACTTTCTGTTCTAATAGGGAACCCTCCTCTCAAAGACCTTTTATCGTAGGAC
TCTGGTTGAAACCAAGTAGCGGTAGTGAAAGGATTCTGCTCAAAGCAGCTAATACTCACTCCAG
CAGTCAACTGTGCGAACAACAAAGCGTTCACCTCGGGGGCGTCTTTGAACTTCAGGCAGGTGCC
AGTGTTTTCGTCAACGTAACAGAAGCATCCCAGGTAATTCATCGAGTAGGGTTTTCTAGCTTTGG
TTTGCTGAAGCTGtaG
anti-CD40_FGK4.5 (SEQ ID NO: 94)
ATGGAAACTGATCGCCTGTTGCTCTGGGTACTTCTTCTGTGGGTGCCTGGGTCCACTGGTGACAC
TGTACTTACACAATCACCCGCTTTGGCCGTTTCTCCTGGTGAACGGGTCACAATTAGTTGCCGAG
CTTCCGATTCTGTATCTACTCTTATGCATTGGTATCAACAAAAACCTGGTCAGCAGCCAAAATTG
CTCATTTATCTTGCTAGTCACTTGGAGTCCGGCGTACCTGCTCGATTCAGCGGTAGTGGGTCTGG
CACAGATTTCACTTTGACCATAGATCCCGTGGAGGCCGATGACACTGCAACCTACTATTGCCAGC
AATCCTGGAACGACCCTTGGACTTTCGGCGGCGGCACCAAGCTGGAACTCAAGCGAGCAGATGC
TGCCCCAACCGTTAGTATATTCCCACCCTCAACCGAACAACTCGCCACAGGAGGCGCTAGTGTC
GTGTGTCTTATGAACAATTTCTATCCACGAGACATTAGCGTCAAGTGGAAAATTGATGGGACAG
AAAGGCGAGATGGAGTTTTGGATTCAGTAACAGACCAGGATTCAAAGGATTCTACCTATAGCAT
GAGCTCCACCTTGAGCCTGACCAAAGCTGATTATGAATCTCATAACCTGTATACTTGTGAAGTGG
TGCATAAGACTTCTAGCTCACCAGTGGTTAAATCTTTTAACCGCAACGAATGTCGGCGCAAGAG
GGGTTCCGGAGAGGGAAGGGGTAGTCTGCTCACCTGCGGCGATGTTGAAGAAAATCCTGGTCCC
ATGGACATTCGGCTCTCTTTGGTATTCCTGGTACTTTTTATAAAGGGGGTGCAATGTGAAGTCCA
GCTCGTGGAAAGCGGTGGGGGCCTGGTTCAGCCCGGTCGCAGCCTTAAACTTAGTTGCGCAGCA
TCCGGATTTACATTTTCTGACTATAACATGGCCTGGGTTCGACAGGCACCCAAAAAAGGGCTGG
AGTGGGTCGCAACTATCATATACGATGGTTCCCGGACATACTATAGAGATTCAGTGAAGGGGCG
CTTTACAATAAGCAGGGACAATGCTAAGTCTACCTTGTATCTTCAGATGGACTCCCTGAGGAGCG
AAGATACAGCAACATATTATTGTGCTACAAACCGCTGGTTGCTGCTTCATTATTTCGACTACTGG
GGTCAGGGCGTCATGGTAACTGTATCAAGCGCCGAGACCACAGCCCCTTCTGTATATCCATTGGC
ACCAGGTACTGCTCTGAAATCCAACTCAATGGTAACCCTTGGATGTCTGGTTAAGGGTTATTTTC
CCGAGCCCGTCACAGTTACTTGGAACTCTGGGGCCCTTTCTAGCGGAGTCCATACCTTTCCCGCC
GTTTTGCAGAGTGGTCTGTACACCCTTACCTCAAGCGTCACAGTTCCATCTAGCACATGGAGCTC
CCAGGCAGTAACTTGTAATGTGGCCCATCCAGCCTCCTCAACTAAGGTAGATAAAAAGATCGTT
CCCAGAGAATGCAATCCATGTGGATGCACCGGGTCTGAGGTCAGCAGTGTGTTCATTTTCCCACC
CAAGACTAAAGATGTATTGACTATTACTCTTACACCCAAAGTAACCTGCGTGGTGGTTGATATTA
GTCAAAATGATCCCGAGGTACGGTTCTCTTGGTTTATCGACGACGTCGAAGTACATACAGCTCAG
ACACACGCTCCCGAGAAACAAAGCAATTCCACTCTTAGGAGCGTGTCCGAGTTGCCAATCGTAC
ATAGGGATTGGCTTAATGGCAAGACCTTTAAGTGTAAGGTCAATTCAGGGGCATTCCCCGCACC
AATAGAGAAGAGTATAAGCAAACCCGAGGGGACACCCAGAGGTCCACAGGTCTATACAATGGC
TCCCCCCAAGGAAGAGATGACCCAAAGTCAAGTCTCAATTACATGTATGGTGAAGGGCTTTTAT
CCACCCGACATATACACTGAGTGGAAGATGAATGGACAGCCCCAAGAGAATTATAAAAACACTC
CCCCTACCATGGACACCGACGGGTCCTATTTTCTTTATAGTAAATTGAACGTGAAAAAGGAGACC
TGGCAACAAGGCAACACTTTCACCTGCTCCGTTCTTCACGAGGGCCTGCATAATCATCATACCGA
AAAGTCTCTCAGTCATTCTCCAGGTAAGtaG
CD40L_2 (Human) (SEQ ID NO: 95)
ATGGAAACAGATACGTTGCTGTTGTGGGTACTTCTCCTTTGGGTCCCTGGCAGCACAGGGGACGA
GAATAGTTTCGAAATGCAGAAGGGCGACCAGAACCCACAGATCGCGGCTCACGTTATATCAGAA
GCAAGTAGTAAGACCACTTCCGTACTTCAGTGGGCTGAAAAAGGATATTACACCATGTCCAACA
ATCTCGTGACACTGGAGAACGGTAAACAACTTACGGTGAAACGACAGGGCCTCTATTACATCTA
CGCTCAGGTGACATTCTGCTCAAATAGGGAGGCTTCTAGTCAAGCGCCCTTCATCGCCAGCCTGT
GCCTCAAATCTCCCGGCCGGTTCGAACGAATCCTGTTGCGAGCGGCCAATACCCATAGCTCAGCT
AAACCTTGCGGCCAGCAGAGTATTCATCTTGGTGGTGTGTTTGAACTTCAGCCGGGAGCATCTGT
GTTCGTCAACGTAACGGACCCTAGCCAAGTGTCTCATGGGACAGGTTTTACATCCTTCGGACTCC
TCAAGTTGtaG
Flt3L (Human) (SEQ ID NO: 96)
ATGACAGTTCTCGCGCCAGCTTGGAGTCCCACCACATACTTGCTTTTGCTTCTGCTTCTGTCCTCT
GGCCTGAGTGGGACCCAAGATTGTTCCTTTCAACATTCCCCAATTAGTTCTGATTTTGCAGTGAA
GATTAGAGAGCTCTCAGACTATCTGCTGCAAGATTATCCTGTCACAGTCGCTTCAAACCTGCAAG
ACGAAGAGCTCTGCGGTGCCTTGTGGCGGTTGGTCTTGGCTCAAAGATGGATGGAGAGACTGAA
AACCGTAGCAGGCAGCAAGATGCAGGGTCTCCTGGAAAGGGTGAACACGGAAATCCATTTTGTG
ACCAAGTGCGCGTTCCAGCCCCCACCGAGTTGTCTCCGGTTTGTTCAAACGAATATATCCCGGTT
GCTCCAGGAAACCTCAGAACAACTGGTGGCTTTGAAACCCTGGATCACAAGACAAAACTTTAGT
CGGTGCCTCGAACTCCAGTGCCAACCAGATTCTTCTACACTTCCCCCCCCGTGGTCCCCGCGCCC
GTTGGAAGCAACGGCCCCAtaG
TGFb TRAP (Human) (SEQ ID NO: 97)
ATGGCCTGGAGTCCTCTGTTTCTGACTCTTATAACTCACTGTGCCGGCAGTTGGGCTATACCCCCT
CATGTACAGAAGTCTGTAAACAACGACATGATTGTAACCGACAATAATGGCGCAGTGAAATTCC
CACAACTGTGTAAGTTCTGTGATGTACGGTTTAGTACATGCGACAATCAAAAAAGCTGTATGTCT
AACTGCTCTATTACATCCATATGTGAAAAACCTCAGGAGGTGTGTGTTGCCGTTTGGCGAAAAAA
TGATGAGAATATCACACTGGAGACAGTATGTCATGACCCTAAACTGCCATACCATGATTTCATAC
TGGAGGACGCCGCCAGTCCTAAGTGCATTATGAAAGAGAAAAAGAAACCCGGTGAAACATTCTT
TATGTGCTCTTGTAGCTCTGACGAGTGTAACGACAACATTATATTCAGCGAGGAGTACAATACAA
GCAACCCCGATATACCACCTCACGTACAAAAAAGTGTCAACAACGATATGATTGTTACCGACAA
TAACGGAGCTGTTAAGTTTCCTCAGTTGTGCAAGTTCTGCGATGTACGATTCTCTACCTGCGACA
ACCAAAAGTCATGTATGTCTAACTGTTCCATAACCTCCATCTGCGAGAAGCCCCAGGAAGTCTGC
GTCGCCGTGTGGCGGAAAAACGACGAGAATATCACTCTTGAAACCGTTTGTCATGATCCTAAAC
TGCCCTATCACGACTTTATTCTGGAAGATGCTGCTTCCCCTAAGTGTATCATGAAAGAAAAGAAG
AAACCTGGGGAGACATTCTTTATGTGTTCATGCTCCTCCGATGAGTGTAACGACAATATCATCTT
CTCTGAGGAATACAACACTTCTAACCCTGATtaG
Fresolimumab (Human) (SEQ ID NO: 98)
ATGGCCTGGTCCCCTCTTTTTCTGACCCTCATCACACACTGTGCAGGCTCATGGGCTGAGACCGT
CTTGACCCAGTCCCCAGGAACTTTGTCTCTGTCTCCTGGTGAAAGAGCTACCCTTAGTTGTCGAG
CCTCTCAGTCCCTTGGTTCTAGCTATCTCGCTTGGTACCAGCAAAAGCCAGGCCAGGCCCCACGA
CTGCTGATCTACGGAGCATCTTCACGGGCTCCCGGCATTCCCGATCGATTTTCCGGATCTGGTAG
TGGTACAGATTTCACACTGACCATATCTCGCCTGGAGCCCGAGGACTTTGCTGTTTATTATTGTC
AGCAGTACGCCGATTCTCCTATCACTTTTGGACAGGGAACCCGCCTGGAGATTAAGCGCACAGT
AGCAGCTCCATCCGTCTTTATCTTTCCACCATCAGATGAACAGCTCAAGAGTGGGACCGCAAGTG
TAGTATGCCTGCTGAACAATTTTTACCCTAGAGAGGCCAAAGTGCAGTGGAAGGTGGATAACGC
CCTCCAGAGTGGCAATAGTCAAGAAAGTGTTACTGAGCAAGATAGTAAGGACTCTACATACTCT
TTGAGTTCTACTTTGACCCTGTCAAAAGCAGATTATGAAAAACATAAGGTGTATGCATGTGAAGT
TACACACCAAGGGTTGTCCTCTCCAGTTACAAAATCTTTTAATAGAGGAGAGTGCCGCCGCAAA
CGCGGTAGTGGAGAAGGTCGAGGCTCACTCTTGACCTGTGGCGACGTGGAAGAAAATCCCGGTC
CTATGGATTGGACTTGGAGGGTATTTTGTCTTTTGGCAGTAACACCTGGAGCTCACCCCCAAGTA
CAGCTCGTCCAATCTGGTGCCGAGGTTAAAAAGCCTGGAAGTTCAGTGAAGGTCTCTTGCAAGG
CATCTGGATACACCTTTTCATCTAACGTCATATCCTGGGTACGGCAAGCCCCAGGACAGGGACTT
GAGTGGATGGGAGGGGTCATCCCCATCGTGGACATTGCTAATTACGCTCAGCGATTCAAAGGGC
GGGTTACTATAACTGCCGACGAGTCTACCTCAACTACCTACATGGAGTTGTCCTCTCTCCGCTCC
GAGGACACTGCTGTATATTACTGTGCCAGCACTCTCGGGTTGGTGTTGGATGCCATGGACTATTG
GGGACAAGGAACCCTGGTGACAGTTAGCTCCGCAAGCACTAAAGGCCCTTCTGTTTTTCCCTTGG
CACCTTGTAGTAGGTCTACCTCTGAGTCTACAGCAGCACTTGGATGCTTGGTTAAGGACTATTTT
CCCGAGCCAGTTACAGTCTCTTGGAACAGTGGTGCCCTCACAAGTGGGGTTCATACCTTCCCCGC
AGTCCTCCAGAGTAGTGGCCTTTACAGCCTCTCATCAGTTGTGACTGTTCCTAGTTCATCACTCGG
TACTAAGACATATACATGTAACGTAGACCACAAGCCAAGCAACACAAAAGTAGACAAACGAGT
CGAATCTAAGTATGGACCCCCTTGTCCCTCCTGTCCTGCTCCCGAGTTCCTTGGGGGCCCTTCCGT
GTTCTTGTTTCCTCCCAAGCCCAAGGATACCCTCATGATCTCACGAACCCCAGAGGTAACATGTG
TGGTTGTTGACGTAAGTCAGGAAGATCCCGAAGTGCAATTTAATTGGTACGTGGATGGCGTCGA
AGTCCATAACGCTAAAACAAAACCCCGAGAGGAACAATTCAATTCCACATATCGGGTGGTGAGT
GTATTGACCGTTCTTCACCAAGATTGGCTGAACGGCAAGGAGTATAAGTGTAAAGTAAGCAACA
AAGGTCTGCCAAGTAGCATAGAAAAAACAATATCTAAAGCTAAGGGCCAACCAAGGGAACCAC
AAGTATATACATTGCCCCCCTCTCAGGAAGAGATGACAAAGAATCAAGTTAGCCTGACCTGTTT
GGTAAAGGGGTTCTATCCCTCAGATATAGCAGTCGAGTGGGAATCTAACGGCCAGCCCGAGAAT
AATTATAAAACAACCCCCCCTGTGTTGGACTCAGACGGCAGCTTCTTTCTCTATTCACGGCTCAC
TGTTGATAAGTCCCGATGGCAGGAGGGGAATGTTTTCAGCTGTAGCGTGATGCACGAAGCTCTC
CACAACCACTATACACAGAAAAGTTTGTCTTTGTCCCTTGGAAAAtaG
TGFb neutralizing peptide (Human) (SEQ ID NO: 99)
ATGAGTACATCCTTTCCAGAGCTGGATCTGGAGAATTTTGAGTATGACGACAGTGCCGAAGCCT
GCTACCTCGGGGACATAGTCGCATTCGGGACAATCTTTTTGTCTGTATTTTACGCCCTGGTGTTTA
CATTTGGCCTGGTTGGAAATCTGTTGGTCGTACTCGCTCTCACCAATTCCCGAAAACCCAAAAGT
ATAACAGACATATACCTGTTGAATCTGGCACTGAGTGACCTTTTGTTCGTCGCCACCCTTCCTTTT
TGGACACACTACCTTATCAGTCACGAGGGGCTTCATAATGCTATGTGCAAGCTCACTACTGCCTT
CTTCTTTATCGGATTCTTCGGGGGTATCTTTTTTATCACAGTTATTAGCATTGACCGATACCTTGC
CATAGTGCTCGCAGCCAACTCAATGAACAACCGCACCGTGCAGCATGGAGTGACTATTTCCTTG
GGTGTGTGGGCCGCTGCTATACTTGTCGCCAGCCCTCAATTCATGTTTACCAAAAGGAAAGACAA
TGAGTGCCTCGGAGATTACCCTGAGGTGTTGCAAGAAATGTGGCCTGTACTTCGAAATAGCGAA
GTGAATATACTCGGCTTTGCTCTTCCTCTGCTCATCATGTCATTCTGTTATTTTCGAATAATCCAA
ACATTGTTCAGCTGTAAGAACCGAAAGAAAGCCCGCGCCGTACGCCTGATTCTGCTCGTTGTGTT
CGCCTTTTTTCTGTTTTGGACTCCTTACAACATAATGATATTCCTGGAGACTCTCAAATTCTATAA
CTTTTTTCCCTCCTGTGATATGAAAAGGGACCTTAGATTGGCTCTCAGTGTCACTGAAACAGTAG
CCTTTAGCCATTGTTGTCTCAACCCTTTCATATATGCATTTGCAGGGGAAAAGTTCCGGCGGTAT
CTCGGACATTTGTATCGGAAGTGCTTGGCCGTGTTGTGTGGTCATCCTGTCCATACCGGATTCTCT
CCTGAGAGTCAACGGAGCCGCCAAGATTCAATCCTGTCCAGTTTCACTCACTATACTTCAGAGGG
GGATGGCAGCCTTCTGCTC
Kyneurinase #1 (SEQ ID NO: 100)
ATGGAGACCGACACTTTGTTGCTGTGGGTACTTTTGTTGTGGGTCCCAGGATCTACCGGGGATAT
GGAACCCTCTCCTCTTGAACTGCCAGTAGACGCCGTGCGCCGCATTGCAGCCGAGTTGAATTGCG
ATCCAACAGATGAACGCGTTGCCCTGAGGCTCGACGAAGAGGATAAATTGTCACATTTCAGGAA
CTGCTTTTACATTCCAAAGATGAGGGATCTTCCATCCATAGATCTTAGCCTCGTGTCCGAGGATG
ACGATGCCATATATTTTCTTGGGAACAGTCTTGGGTTGCAGCCAAAAATGGTACGGACATATCTC
GAAGAGGAGCTGGACAAATGGGCTAAAATGGGTGCTTACGGCCACGACGTGGGAAAACGCCCC
TGGATAGTTGGCGACGAATCTATCGTGAGTCTTATGAAAGATATAGTTGGAGCACATGAGAAAG
AAATTGCACTGATGAATGCCCTTACTATCAATCTGCATCTCCTCTTGCTTTCATTCTTTAAGCCCA
CTCCTAAACGCCACAAAATACTTTTGGAAGCAAAAGCCTTTCCAAGCGACCACTACGCTATTGA
GTCACAAATACAACTCCATGGACTTGATGTGGAAAAGTCTATGCGGATGGTAAAACCACGCGAA
GGCGAGGAGACCCTTCGAATGGAGGACATACTTGAGGTCATCGAAGAAGAAGGAGATAGTATA
GCAGTTATCCTTTTCAGCGGGCTGCACTTCTACACAGGTCAACTCTTTAACATTCCAGCTATTACT
AAGGCAGGCCACGCTAAAGGATGCTTCGTGGGCTTTGACCTTGCACACGCAGTAGGAAACGTAG
AGCTCCGCTTGCACGATTGGGGCGTTGATTTCGCCTGCTGGTGTTCATATAAGTATCTTAACTCA
GGAGCTGGTGGGTTGGCAGGCGCATTCGTACACGAGAAACACGCTCATACCGTAAAGCCTGCAC
TGGTAGGGTGGTTCGGACACGATCTCTCTACCCGCTTCAATATGGATAATAAACTCCAGCTTATA
CCTGGCGCCAATGGATTCAGGATCTCAAATCCTCCTATTTTGCTCGTTTGCAGTTTGCACGCATCT
CTTGAGGTGTTCCAGCAGGCTACCATGACTGCACTCCGCCGGAAGTCAATCCTTTTGACCGGATA
CTTGGAGTATATGCTGAAACATTATCACTCAAAAGATAACACTGAGAATAAGGGCCCCATAGTA
AACATTATCACTCCATCTCGGGCTGAAGAGCGCGGCTGCCAACTCACATTGACTTTTTCCATTCC
CAAGAAGTCAGTGTTCAAAGAGTTGGAGAAACGGGGGGTTGTATGTGATAAGCGGGAGCCAGA
TGGAATCCGCGTTGCCCCAGTCCCCCTCTATAATTCTTTTCACGATGTATACAAGTTTATTAGACT
GCTGACAAGTATCTTGGACTCATCTGAGCGATCTtaG
Kyneurinase #2 (SEQ ID NO: 101)
ATGGAACCCTCTCCTCTTGAACTGCCAGTAGACGCCGTGCGCCGCATTGCAGCCGAGTTGAATTG
CGATCCAACAGATGAACGCGTTGCCCTGAGGCTCGACGAAGAGGATAAATTGTCACATTTCAGG
AACTGCTTTTACATTCCAAAGATGAGGGATCTTCCATCCATAGATCTTAGCCTCGTGTCCGAGGA
TGACGATGCCATATATTTTCTTGGGAACAGTCTTGGGTTGCAGCCAAAAATGGTACGGACATATC
TCGAAGAGGAGCTGGACAAATGGGCTAAAATGGGTGCTTACGGCCACGACGTGGGAAAACGCC
CCTGGATAGTTGGCGACGAATCTATCGTGAGTCTTATGAAAGATATAGTTGGAGCACATGAGAA
AGAAATTGCACTGATGAATGCCCTTACTATCAATCTGCATCTCCTCTTGCTTTCATTCTTTAAGCC
CACTCCTAAACGCCACAAAATACTTTTGGAAGCAAAAGCCTTTCCAAGCGACCACTACGCTATTG
AGTCACAAATACAACTCCATGGACTTGATGTGGAAAAGTCTATGCGGATGGTAAAACCACGCGA
AGGCGAGGAGACCCTTCGAATGGAGGACATACTTGAGGTCATCGAAGAAGAAGGAGATAGTAT
AGCAGTTATCCTTTTCAGCGGGCTGCACTTCTACACAGGTCAACTCTTTAACATTCCAGCTATTAC
TAAGGCAGGCCACGCTAAAGGATGCTTCGTGGGCTTTGACCTTGCACACGCAGTAGGAAACGTA
GAGCTCCGCTTGCACGATTGGGGCGTTGATTTCGCCTGCTGGTGTTCATATAAGTATCTTAACTC
AGGAGCTGGTGGGTTGGCAGGCGCATTCGTACACGAGAAACACGCTCATACCGTAAAGCCTGCA
CTGGTAGGGTGGTTCGGACACGATCTCTCTACCCGCTTCAATATGGATAATAAACTCCAGCTTAT
ACCTGGCGCCAATGGATTCAGGATCTCAAATCCTCCTATTTTGCTCGTTTGCAGTTTGCACGCATC
TCTTGAGGTGTTCCAGCAGGCTACCATGACTGCACTCCGCCGGAAGTCAATCCTTTTGACCGGAT
ACTTGGAGTATATGCTGAAACATTATCACTCAAAAGATAACACTGAGAATAAGGGCCCCATAGT
AAACATTATCACTCCATCTCGGGCTGAAGAGCGCGGCTGCCAACTCACATTGACTTTTTCCATTC
CCAAGAAGTCAGTGTTCAAAGAGTTGGAGAAACGGGGGGTTGTATGTGATAAGCGGGAGCCAG
ATGGAATCCGCGTTGCCCCAGTCCCCCTCTATAATTCTTTTCACGATGTATACAAGTTTATTAGAC
TGCTGACAAGTATCTTGGACTCATCTGAGCGATCTtaG
VEGF (SEQ ID NO: 102)
ATGAATTTCTTGCTGAGCTGGGTGCATTGGACACTCGCATTGTTGCTGTACTTGCACCATGCCAA
GTGGTCCCAGGCTGCACCCACTACTGAGGGCGAGCAAAAGTCTCATGAGGTGATTAAATTTATG
GACGTTTACCAACGATCATACTGTCGGCCAATCGAAACCCTCGTAGATATATTCCAGGAGTACCC
AGACGAGATCGAATACATTTTCAAGCCCTCATGTGTCCCATTGATGCGATGTGCTGGGTGCTGTA
ACGACGAAGCACTTGAATGTGTCCCCACCTCCGAGAGTAACATCACAATGCAAATAATGAGAAT
CAAGCCCCACCAATCCCAACATATCGGTGAAATGTCATTCCTTCAGCATTCCCGCTGCGAGTGCC
GGCCTAAGAAGGACCGCACCAAACCAGAGAACCATTGTGAACCCTGTTCTGAGAGACGGAAGC
ACTTGTTCGTACAGGACCCTCAAACATGCAAGTGCAGCTGTAAGAATACCGACTCACGGTGTAA
AGCTAGGCAACTGGAGCTTAATGAAAGGACCTGCCGATGCGATAAACCCAGGAGGtaa
GM-CSF (SEQ ID NO: 103)
ATGTGGTTGCAGAATTTGCTCTTCCTGGGGATTGTGGTCTACAGCCTCTCCGCACCTACCCGCTCT
CCTATCACAGTTACAAGACCCTGGAAACATGTGGAGGCCATTAAAGAAGCATTGAATTTGTTGG
ACGATATGCCCGTCACCCTGAATGAAGAAGTAGAAGTTGTTTCTAATGAGTTCAGCTTTAAAAA
ATTGACCTGTGTGCAGACACGGCTTAAAATTTTTGAACAGGGACTTAGAGGAAACTTTACTAAG
CTGAAGGGGGCACTTAACATGACAGCTTCTTATTATCAGACCTATTGTCCTCCAACACCTGAAAC
CGACTGTGAAACACAGGTAACCACTTACGCCGATTTTATTGATTCTTTGAAAACATTCCTCACCG
ATATACCATTTGAGTGTAAGAAGCCAGGCCAAAAGtaG
Anti-PD1 (SEQ ID NO: 104)
ATGGAAACTGACACACTTCTTCTGTGGGTCTTGCTCCTGTGGGTCCCAGGCTCTACTGGTGACAG
TCCTGATAGGCCATGGAACCCACCTACCTTTAGTCCAGCCTTGCTCGTCGTAACCGAAGGGGACA
ACGCTACATTCACCTGCTCTTTTAGCAATACTTCTGAGAGTTTTCATGTAGTCTGGCATCGGGAG
AGTCCATCCGGACAAACAGATACTTTGGCCGCTTTTCCAGAGGATAGGTCTCAACCTGGGCAAG
ACGCAAGGTTTCGAGTCACACAGCTTCCTAACGGGAGAGATTTTCACATGTCTGTAGTTCGGGCA
CGCCGAAATGATTCTGGCACATATGTTTGCGGTGTGATCTCACTTGCTCCAAAGATTCAAATAAA
GGAGAGCCTTCGCGCCGAGTTGCGGGTGACTGAGCGGGAGCCCAAGTCCTGCGACAAAACCCAT
ACTTGTCCACCCTGTGGCGGCGGGTCATCCGGTGGCGGGTCTGGGGGGCAACCAAGAGAGCCAC
AGGTATATACTCTTCCCCCCAGCAGAGAAGAAATGACAAAAAACCAAGTGTCCCTGACATGTCT
GGTTAAAGGATTTTATCCCAGTGACATTGCTGTAGAATGGGAATCCAATGGTCAACCCGAGAAT
AACTACAAAACCACTCCTCCAGTATTGGACAGTGACGGTTCCTTCTTCCTCTATTCCAAACTTAC
AGTGGATAAATCCCGCTGGCAGCAAGGGAATGTATTCAGCTGTAGTGTCATGCACGAAGCTCTT
CATAACCATTATACACAGAAATCTCTTTCCCTGAGCCCAGGTAAAtaG
Adenosine Deaminase (ADA) #1 (Mouse) (SEQ ID NO: 105)
ATGGAGACTGATACACTTTTGCTCTGGGTTTTGCTCTTGTGGGTACCAGGGTCTACTGGAGATGC
ACAAACTCCTGCATTCAACAAGCCTAAGGTAGAGCTTCATGTCCATTTGGACGGAGCCATAAAA
CCTGAAACCATACTCTATTTCGGCAAGAAACGGGGTATAGCACTTCCCGCTGATACCGTGGAAG
AGTTGAGAAATATCATTGGCATGGACAAACCTCTTAGCCTGCCTGGCTTTCTTGCAAAGTTCGAC
TACTATATGCCAGTTATAGCAGGGTGTAGAGAAGCAATAAAGCGAATCGCCTATGAGTTCGTTG
AGATGAAGGCTAAAGAAGGAGTTGTTTACGTGGAAGTCCGGTACTCACCTCATCTGCTTGCTAAT
AGCAAGGTGGACCCAATGCCATGGAATCAAACTGAAGGTGATGTAACCCCTGACGATGTGGTCG
ATTTGGTCAATCAAGGTCTCCAAGAAGGCGAGCAGGCTTTCGGCATTAAGGTAAGAAGTATATT
GTGCTGTATGCGACATCAACCTTCATGGTCCCTGGAGGTCCTCGAATTGTGCAAAAAGTACAATC
AAAAAACAGTGGTCGCAATGGATCTCGCTGGAGATGAGACCATAGAAGGTTCCTCTCTTTTCCCC
GGTCATGTCGAAGCATATGAAGGGGCTGTCAAAAATGGTATCCACCGCACCGTCCACGCAGGGG
AAGTAGGGTCCCCAGAAGTAGTCAGGGAAGCCGTTGACATTTTGAAAACAGAAAGAGTCGGGC
ATGGCTACCATACAATAGAGGACGAAGCCTTGTACAATCGACTTTTGAAAGAAAATATGCACTT
CGAGGTCTGTCCCTGGAGTTCATATCTCACCGGAGCATGGGACCCCAAAACAACCCACGCCGTC
GTACGCTTCAAGAATGATAAGGCAAACTACAGTTTGAATACAGATGATCCACTGATATTCAAGT
CAACACTTGACACTGACTACCAGATGACAAAAAAAGATATGGGTTTCACCGAAGAAGAGTTCAA
GAGATTGAACATTAACGCAGCAAAAAGCTCCTTCCTGCCAGAGGAAGAGAAAAAAGAATTGCTT
GAAAGGTTGTATCGAGAATACCAA
Adenosine Deaminase (ADA) #2 (Mouse) (SEQ ID NO: 106)
ATGGCACAAACTCCAGCTTTTAATAAGCCCAAAGTGGAACTTCATGTTCATCTGGATGGGGCAAT
TAAGCCCGAAACTATATTGTACTTTGGCAAAAAGAGGGGTATTGCCCTGCCAGCAGATACCGTT
GAGGAGCTTCGCAACATCATTGGGATGGACAAGCCCCTCTCTCTGCCAGGTTTTCTCGCTAAATT
CGATTATTATATGCCTGTTATTGCTGGTTGCCGGGAGGCCATCAAGAGGATAGCCTACGAGTTTG
TTGAGATGAAGGCCAAAGAGGGCGTGGTGTACGTAGAGGTCAGATACAGCCCTCACCTGCTTGC
CAACAGCAAGGTGGACCCAATGCCCTGGAACCAAACCGAGGGGGATGTCACTCCCGACGACGTT
GTAGACCTCGTAAATCAGGGCCTTCAAGAGGGCGAGCAGGCATTTGGCATAAAAGTCCGGTCTA
TACTCTGCTGTATGAGGCACCAACCCTCCTGGTCTTTGGAGGTACTTGAGTTGTGTAAGAAATAC
AATCAAAAGACTGTAGTCGCCATGGATCTTGCAGGCGATGAAACCATCGAGGGTAGCTCCTTGT
TCCCTGGACATGTTGAAGCCTACGAGGGGGCCGTAAAAAATGGGATACACAGGACTGTCCACGC
TGGTGAAGTCGGAAGCCCAGAGGTGGTAAGGGAGGCAGTTGACATACTCAAGACAGAGCGGGT
TGGACACGGATACCACACAATTGAGGACGAGGCCCTGTATAACCGCCTCCTCAAAGAGAACATG
CATTTTGAGGTGTGTCCTTGGTCCAGCTACCTGACTGGTGCTTGGGACCCTAAAACAACTCACGC
CGTGGTCCGGTTCAAGAACGATAAAGCCAATTACTCTTTGAATACCGACGACCCCCTCATATTCA
AATCAACATTGGATACCGACTACCAAATGACCAAAAAGGATATGGGGTTTACTGAAGAGGAGTT
CAAGAGGCTCAACATAAATGCCGCTAAATCCTCCTTTCTCCCCGAGGAAGAAAAAAAAGAACTC
CTTGAGCGGCTGTATAGGGAGTATCAA
4-1BBL #1 (Mouse) (SEQ ID NO: 107)
ATGGAAACAGATACACTCTTGCTCTGGGTACTGCTTCTGTGGGTCCCCGGCTCTACTGGGGATGA
AGATGATGTAACTACTACAGAAGAACTCGCTCCCGCTCTTGTCCCCCCACCCAAGGGTACCTGCG
CCGGTTGGATGGCTGGCATCCCAGGACATCCAGGTCACAACGGTACCCCCGGAAGAGATGGTCG
GGATGGAACTCCCGGCGAGAAGGGCGAAAAAGGGGATGCAGGGCTTCTGGGACCTAAAGGTGA
AACAGGGGACGTTGGAATGACTGGTGCAGAAGGGCCTCGCGGCTTTCCTGGCACCCCTGGGAGG
AAAGGAGAGCCCGGAGAGCTCCAGAGAACTGAACCTCGGCCTGCACTCACTATAACTACTTCCC
CTAATCTTGGGACCCGCGAGAACAACGCCGATCAGGTTACACCTGTAAGCCATATCGGGTGCCC
CAATACTACCCAGCAAGGGAGTCCCGTGTTCGCAAAGCTTTTGGCTAAAAACCAAGCATCCCTG
TGTAACACTACTCTTAATTGGCATTCACAAGACGGTGCTGGTAGCTCTTATCTTTCTCAGGGGCT
GCGGTACGAAGAAGATAAGAAGGAATTGGTTGTGGATTCTCCAGGACTCTATTATGTCTTTCTCG
AATTGAAGCTCAGTCCCACCTTCACAAACACTGGACACAAAGTCCAGGGCTGGGTAAGTCTGGT
ACTCCAAGCAAAGCCCCAGGTTGACGATTTCGACAATTTGGCACTCACCGTAGAGCTTTTCCCAT
GCTCCATGGAAAATAAACTTGTTGATCGGTCATGGTCACAGCTCTTGCTGCTTAAGGCAGGGCAT
CGCCTCTCAGTGGGTCTGAGAGCTTATTTGCATGGTGCACAAGATGCTTACAGGGATTGGGAATT
GTCCTACCCAAACACTACAAGTTTCGGGTTGTTCCTTGTCAAACCTGATAACCCATGGGAGtaG
4-1BBL #2 (Mouse) (SEQ ID NO: 108)
ATGGAAACTGATACACTCCTCCTGTGGGTCCTTCTTTTGTGGGTGCCCGGATCAACCGGCGATGG
CTGGATGGCAGGCATCCCAGGACACCCAGGACACAACGGTACTCCAGGTCGAGACGGTCGGGAT
GGGACTCCTGGGGAGAAAGGCGAGAAAGGGGACGCTGGTTTGCTCGGTCCTAAGGGGGAAACC
GGGGATGTAGGAATGACAGGGGCTGAAGGGCCTCGGGGATTTCCTGGGACACCAGGCAGGAAG
GGTGAACCAGGGGAGGCCCTCCAGCGCACCGAGCCACGGCCAGCTCTGACCATAACAACAAGT
CCAAACCTGGGCACACGCGAAAACAATGCTGACCAGGTGACTCCTGTAAGTCACATCGGATGCC
CTAACACTACACAACAGGGCTCTCCTGTATTTGCAAAGCTTCTCGCAAAAAATCAAGCATCACTT
TGTAATACAACCCTGAACTGGCATTCTCAGGACGGAGCAGGGTCCTCTTATTTGTCTCAAGGGCT
CCGCTACGAAGAAGATAAAAAGGAATTGGTTGTTGACAGTCCAGGTTTGTATTATGTGTTTTTGG
AACTTAAGCTGTCACCAACCTTCACTAACACCGGCCACAAGGTCCAAGGCTGGGTTAGTCTTGTT
TTGCAAGCCAAACCTCAAGTGGATGATTTTGACAATCTGGCTTTGACTGTTGAGCTTTTTCCATG
CAGTATGGAGAATAAACTGGTTGATCGGTCATGGTCACAGCTCCTTCTGCTCAAGGCCGGACAT
AGGCTGAGTGTGGGACTTCGGGCCTACTTGCACGGCGCCCAGGACGCATACCGAGACTGGGAAC
TCAGCTACCCTAACACAACTTCTTTTGGGTTGTTCCTTGTCAAACCCGATAATCCTTGGGAAtaG
HPGE2 #1 (Mouse) (SEQ ID NO: 109)
ATGGAGACTGATACTTTGCTCCTGTGGGTTCTTCTCCTGTGGGTTCCTGGTTCCACAGGGGATAT
GCATGTCAATGGCAAGGTAGCACTCGTGACTGGGGCTGCACAGGGTATCGGGAAAGCTTTTGCC
GAGGCCCTGTTGCTGCATGGCGCCAAGGTCGCTTTGGTAGATTGGAACTTGGAGGCTGGAGTTA
AATGCAAAGCTGCACTCGACGAACAATTTGAGCCTCAAAAAACCCTCTTTGTGCAGTGTGACGTT
GCTGACCAAAAGCAACTCAGGGACACATTCAGGAAGGTCGTAGACCATTTCGGACGCCTCGATA
TACTCGTTAATAATGCCGGGGTAAACAACGAAAAGAACTGGGAACAAACATTGCAAATCAACCT
GGTAAGTGTCATTAGCGGAACTTATCTGGGTCTTGATTATATGAGCAAGCAGAACGGGGGCGAG
GGCGGGATCATTATCAACATGTCAAGTCTTGCCGGATTGATGCCAGTTGCTCAGCAGCCTGTTTA
CTGTGCCAGCAAGCACGGTATTATTGGGTTTACCCGGAGTGCCGCCATGGCCGCAAATCTTATGA
AGAGTGGGGTAAGACTGAATGTTATCTGCCCAGGTTTCGTAGATACCCCAATCCTGGAGAGCAT
CGAGAAGGAGGAAAATATGGGACAATACATTGAATATAAAGATCAAATCAAGGCTATGATGAA
GTTCTACGGGGTTCTGCATCCATCCACAATTGCCAACGGGCTCATTAATCTGATTGAGGACGACG
CCTTGAACGGAGCTATAATGAAAATCACAGCTTCCAAAGGCATTCACTTCCAAGATTATGATATA
TCACCCTTGCTTGTCAAGGCTCCTCTGACAAGT
HPGE2 #2 (Mouse) (SEQ ID NO: 110)
ATGCATGTCAATGGCAAGGTAGCACTCGTGACTGGGGCTGCACAGGGTATCGGGAAAGCTTTTG
CCGAGGCCCTGTTGCTGCATGGCGCCAAGGTCGCTTTGGTAGATTGGAACTTGGAGGCTGGAGTT
AAATGCAAAGCTGCACTCGACGAACAATTTGAGCCTCAAAAAACCCTCTTTGTGCAGTGTGACG
TTGCTGACCAAAAGCAACTCAGGGACACATTCAGGAAGGTCGTAGACCATTTCGGACGCCTCGA
TATACTCGTTAATAATGCCGGGGTAAACAACGAAAAGAACTGGGAACAAACATTGCAAATCAAC
CTGGTAAGTGTCATTAGCGGAACTTATCTGGGTCTTGATTATATGAGCAAGCAGAACGGGGGCG
AGGGCGGGATCATTATCAACATGTCAAGTCTTGCCGGATTGATGCCAGTTGCTCAGCAGCCTGTT
TACTGTGCCAGCAAGCACGGTATTATTGGGTTTACCCGGAGTGCCGCCATGGCCGCAAATCTTAT
GAAGAGTGGGGTAAGACTGAATGTTATCTGCCCAGGTTTCGTAGATACCCCAATCCTGGAGAGC
ATCGAGAAGGAGGAAAATATGGGACAATACATTGAATATAAAGATCAAATCAAGGCTATGATG
AAGTTCTACGGGGTTCTGCATCCATCCACAATTGCCAACGGGCTCATTAATCTGATTGAGGACGA
CGCCTTGAACGGAGCTATAATGAAAATCACAGCTTCCAAAGGCATTCACTTCCAAGATTATGAT
ATATCACCCTTGCTTGTCAAGGCTCCTCTGACAAGT
Human IL-15 Polypeptide Sequence (SEQ ID NO: 199)
MVLGTIDLCSCFSAGLPKTEANWVNVISDLKKIEDLIQSMHIDATLYTESDVHPSCKVTAMKCFLLEL
QVISLESGDASIHDTVENLIILANNSLSSNGNVTESGCKECEELEEKNIKEFLQSFVHIVQMFINTS
Human IL-15 Polypeptide Sequence (SEQ ID NO: 224)
NWVNVISDLKKIEDLIQSMHIDATLYTESDVHPSCKVTAMKCFLLELQVISLESGDASIHDTVENLIIL
ANNSLSSNGNVTESGCKECEELEEKNIKEFLQSFVHIVQMFINTS
Human IL-15Rx Polypeptide Sequence (SEQ ID NO: 200)
MAPRRARGCRTLGLPALLLLLLLRPPATRGITCPPPMSVEHADIWVKSYSLYSRERYICNSGFKRKAG
TSSLTECVLNKATNVAHWTTPSLKCIRDPALVHQRPAPPSTVTTAGVTPQPESLSPSGKEPAASSPSSN
NTAATTAAIVPGSQLMPSKSPSTGTTEISSHESSHGTPSQTTAKNWELTASASHQPPGVYPQGHSDTT
VAISTSTVLLCGLSAVSLLACYLKSRQTPPLASVEMEAMEALPVTWGTSSRDEDLENCSHHL
Human IL-15Rx sushi domain Polypeptide Sequence (SEQ ID NO: 201)
ITCPPPMSVEHADIWVKSYSLYSRERYICNSGFKRKAGTSSLTECVLNKATNVAHWTTPSLKCIR
Human IL-15/IL-15Rx sushi domain Polypeptide Sequence (SEQ ID NO: 202)
MDWTWILFLVAAATRVHSNWVNVISDLKKIEDLIQSMHIDATLYTESDVHPSCKVTAMKCFLLELQ
VISLESGDASIHDTVENLIILANNSLSSNGNVTESGCKECEELEEKNIKEFLQSFVHIVQMFINTSSGGSG
GGGSGGGSGGGGSLQITCPPPMSVEHADIWVKSYSLYSRERYICNSGFKRKAGTSSLTECVLNKATN
VAHWTTPSLKCIR
IL-12 (IL-12p70) Polypeptide Sequence (SEQ ID NO: 203)
MCHQQLVISWFSLVFLASPLVAIWELKKDVYVVELDWYPDAPGEMVVLTCDTPEEDGITWTLDQSS
EVLGSGKTLTIQVKEFGDAGQYTCHKGGEVLSHSLLLLHKKEDGIWSTDILKDQKEPKNKTFLRCEA
KNYSGRFTCWWLTTISTDLTFSVKSSRGSSDPQGVTCGAATLSAERVRGDNKEYEYSVECQEDSACP
AAEESLPIEVMVDAVHKLKYENYTSSFFIRDIIKPDPPKNLQLKPLKNSRQVEVSWEYPDTWSTPHSY
FSLTFCVQVQGKSKREKKDRVFTDKTSATVICRKNASISVRAQDRYYSSSWSEWASVPCSGGGSGGG
SGGGSGGGSRNLPVATPDPGMFPCLHHSQNLLRAVSNMLQKARQTLEFYPCTSEEIDHEDITKDKTS
TVEACLPLELTKNESCLNSRETSFITNGSCLASRKTSFMMALCLSSIYEDLKMYQVEFKTMNAKLLM
DPKRQIFLDQNMLAVIDELMQALNFNSETVPQKSSLEEPDFYKTKIKLCILLHAFRIRAVTIDRVMSYL
NAS

### Secretion Signals and Signal-Anchors

The one or more effector molecules of the chimeric proteins provided for herein can be secretable effector molecules having a secretion signal peptide (also referred to as a signal peptide or signal sequence) at the chimeric protein's N-terminus (e.g., an effector molecule's N-terminus for S—C-MT) that direct newly synthesized proteins destined for secretion or membrane localization (also referred to as membrane insertion) to the proper protein processing pathways. For chimeric proteins having the formula MT-C—S, a membrane tethering domain generally has a signal-anchor sequence (e.g., signal-anchor sequences of a Type II transmembrane protein) that direct newly synthesized proteins destined for membrane localization to the proper protein processing pathways. For chimeric proteins having the formula S—C-MT, a membrane tethering domain having a reverse signal-anchor sequence (e.g., signal-anchor sequences of certain Type III transmembrane proteins) can be used, generally without a separate secretion signal peptide, that direct newly synthesized proteins destined for membrane localization to the proper protein processing pathways.

In general, for all membrane-cleavable chimeric proteins described herein, the one or more effector molecules are secretable effector molecules (referred to as “S” in the formula S—C-MT or MT-C—S). In embodiments with two or more chimeric proteins, each chimeric protein can comprise a secretion signal. In embodiments with two or more chimeric proteins, each chimeric protein can comprise a secretion signal such that each effector molecule is capable of secretion from an engineered cell following cleavage of the protease cleavage site.

The secretion signal peptide operably associated with an effector molecule can be a native secretion signal peptide (e.g., the secretion signal peptide generally endogenously associated with the given effector molecule). The secretion signal peptide operably associated with an effector molecule can be a non-native secretion signal peptide native secretion signal peptide. Non-native secretion signal peptides can promote improved expression and function, such as maintained secretion, in particular environments, such as tumor microenvironments. Non-limiting examples of non-native secretion signal peptide are shown in Table 3.

A secretion signal peptide can be an IgE signal peptide. An IgE signal peptide can include the amino acid sequence MDWTWILFLVAAATRVHS (SEQ ID NO: 228). An IgE signal peptide can be encoded by a polynucleotide sequence that includes the sequence ATGGACTGGACTTGGATACTCTTTCTGGTCGCTGCCGCCACACGGGTGCACTCT (SEQ ID NO: 229). An IgE signal peptide can be encoded by a polynucleotide sequence that includes a sequence that is at least 90%, at least 91%, at least 92%, at least 93%, at least 94%, at least 95%, at least 96%, at least 97%, at least 98%, or at least 99% identical to

(SEQ ID NO: 229)
ATGGACTGGACTTGGATACTCTTTCTGGTCGCTGCCGCCACACGG
GTGCACTCT.

A secretion signal peptide of the membrane-cleavable chimeric protein (e.g., IL-15) can be an IgE signal peptide. An IgE signal peptide of the membrane-cleavable chimeric protein (e.g., IL-15) can include the amino acid sequence MDWTWILFLVAAATRVHS (SEQ ID NO: 228). An IgE signal peptide of the membrane-cleavable chimeric protein (e.g., IL-15) can be encoded by a polynucleotide sequence that includes the sequence ATGGACTGGACTTGGATACTCTTTCTGGTCGCTGCCGCCACACGGGTGCACTCT (SEQ ID NO: 229). An IgE signal peptide of the membrane-cleavable chimeric protein (e.g., IL-15) can be encoded by a polynucleotide sequence that includes a sequence that is at least 90%, at least 91%, at least 92%, at least 93%, at least 94%, at least 95%, at least 96%, at least 97%, at least 98%, or at least 99% identical to

(SEQ ID NO: 229)
ATGGACTGGACTTGGATACTCTTTCTGGTCGCTGCCGCCACACGG
GTGCACTCT.

TABLE 3
Exemplary Signal Secretion Peptides

| Name | Protein SEQUENCE | Source (Uniprot) | DNA SEQUENCE |
|---|---|---|---|
| IL-12 | MCHQQLVISWFSLV | P29460 | ATGTGTCACCAGCAGCTCGTTATATC |
| | FLASPLVA (SEQ ID | | CTGGTTTAGTTTGGTGTTTCTCGCTT |
| | NO: 112) | | CACCCCTGGTGGCA (SEQ ID NO: 31) |
| IL-12 (Codon | MCHQQLVISWFSLV | — | ATGTGCCATCAGCAACTCGTCATCTC |
| Optimized) | FLASPLVA (SEQ ID | | CTGGTTCTCCCTTGTGTTCCTCGCTTC |
| | NO: 112) | | CCCTCTGGTCGCC (SEQ ID NO: 32) |
| IL-2 (Optimized) | MQLLSCIALILALV | — | ATGCAACTGCTGTCATGTATCGCACT |
| | (SEQ ID NO: 113) | | CATCCTGGCGCTGGTA (SEQ ID NO: |
| | | | 33) |
| IL-2 (Native) | MYRMQLLSCIALSL | P60568 | ATGTATCGGATGCAACTTTTGAGCTG |
| | ALVTNS (SEQ ID | | CATCGCATTGTCTCTGGCGCTGGTGA |
| | NO: 114) | | CAAATTCC (SEQ ID NO: 34) |
| Trypsinogen-2 | MNLLLILTFVAAAV | P07478 | ATGAATCTCTTGCTCATACTTACGTT |
| | A (SEQ ID NO: 115) | | TGTCGCTGCTGCCGTTGCG (SEQ ID |
| | | | NO: 35) |
| Gaussia | MGVKVLFALICIAV | — | ATGGGCGTGAAGGTCTTGTTTGCCCT |
| Luciferase | AEA (SEQ ID NO: | | TATCTGCATAGCTGTTGCGGAGGCG |
| | 116) | | (SEQ ID NO: 36) |
| CD5 | MPMGSLQPLATLYL | P06127 | ATGCCGATGGGGAGCCTTCAACCTTT |
| | LGMLVASCLG (SEQ | | GGCAACGCTTTATCTTCTGGGGATGT |
| | ID NO: 117) | | TGGTTGCTAGTTGCCTTGGG (SEQ ID |
| | | | NO: 37) |
| IgKVII (mouse) | METDTLLLWVLLL | | ATGGAAACTGACACGTTGTTGCTGT |
| | WVPGSTGD (SEQ ID | | GGGTATTGCTCTTGTGGGTCCCAGGA |
| | NO: 118) | | TCTACGGGCGAC (SEQ ID NO: 38) |
| IgKVII (human) | MDMRVPAQLLGLL | P01597 | ATGGATATGAGGGTTCCCGCCCAGC |
| | LLWLRGARC (SEQ | | TTTTGGGGCTGCTTTTGTTGTGGCTT |
| | ID NO: 119) | | CGAGGGGCTCGGTGT (SEQ ID NO: |
| | | | 39) |
| VSV-G | MKCLLYLAFLFIGV | — | ATGAAGTGTCTGTTGTACCTGGCGTT |
| | NC (SEQ ID NO: 120) | | TCTGTTCATTGGTGTAAACTGT (SEQ |
| | | | ID NO: 40) |
| Prolactin | MNIKGSPWKGSLLL | P01236 | ATGAATATCAAAGGAAGTCCGTGGA |
| | LLVSNLLLCQSVAP | | AGGGTAGTCTCCTGCTGCTCCTCGTA |
| | (SEQ ID NO: 121) | | TCTAACCTTCTCCTTTGTCAATCCGT |
| | | | GGCACCC (SEQ ID NO: 41) |
| Serum albumin | MKWVTFISLLFLFSS | P02768 | ATGAAATGGGTAACATTCATATCAC |
| preproprotein | AYS (SEQ ID NO: | | TTCTCTTTCTGTTCAGCTCTGCGTATT |
| | 122) | | CT (SEQ ID NO: 42) |
| Azurocidin | MTRLTVLALLAGLL | 20160 | ATGACAAGGCTTACTGTTTTGGCTCT |
| preproprotein | ASSRA (SEQ ID NO: | | CCTCGCTGGACTCTTGGCTTCCTCCC |
| | 123) | | GAGCA (SEQ ID NO: 43) |
| Osteonectin | MRA WIFFLLCLAGR | P09486 | ATGAGGGCTTGGATTTTTTTTCTGCT |
| (BM40) | ALA (SEQ ID NO: | | CTGCCTTGCCGGTCGAGCCCTGGCG |
| | 124) | | (SEQ ID NO: 44) |
| CD33 | MPLLLLLPLLWAGA | P20138 | ATGCCTCTTCTGCTTTTGCTTCCTCTT |
| | LA (SEQ ID NO: 125) | | TTGTGGGCAGGTGCCCTCGCA (SEQ |
| | | | ID NO: 45) |
| IL-6 | MNSFSTSAFGPVAFS | P05231 | ATGAACTCTTTCTCAACCTCTGCGTT |
| | LGLLLVLPAAFPAP | | TGGTCCGGTCGCTTTCTCCCTTGGGC |
| | (SEQ ID NO: 126) | | TCCTGCTTGTCTTGCCAGCAGCGTTT |
| | | | CCTGCGCCA (SEQ ID NO: 46) |
| IL-8 | MTSKLAVALLAAFL | P10145 | ATGACAAGTAAACTGGCGGTAGCCT |
| | ISAALC (SEQ ID NO: | | TGCTCGCGGCCTTTTTGATTTCCGCA |
| | 127) | | GCCCTTTGT (SEQ ID NO: 47) |
| CCL2 | MKVSAALLCLLLIA | P13500 | ATGAAGGTAAGTGCAGCGTTGCTTT |
| | ATFIPQGLA (SEQ ID | | GCCTTCTCCTCATTGCAGCGACCTTT |
| | NO: 128) | | ATTCCTCAAGGGCTGGCC (SEQ ID |
| | | | NO: 48) |
| TIMP2 | MGAAARTLRLALGL | P16035 | ATGGGAGCGGCAGCTAGAACACTTC |
| | LLLATLLRPADA | | GACTTGCCCTTGGGCTCTTGCTCCTT |
| | (SEQ ID NO: 129) | | GCAACCCTCCTTAGACCTGCCGACG |
| | | | CA (SEQ ID NO: 49) |
| VEGFB | MSPLLRRLLLAALL | P49765 | ATGTCACCGTTGTTGCGGAGATTGCT |
| | QLAPAQA (SEQ ID | | GTTGGCCGCACTTTTGCAACTGGCTC |
| | NO: 130) | | CTGCTCAAGCC (SEQ ID NO: 50) |
| Osteoprotegerin | MNNLLCCALVFLDI | O00300 | ATGAATAACCTGCTCTGTTGTGCGCT |
| | SIKWTTQ (SEQ ID | | CGTGTTCCTGGACATTTCTATAAAAT |
| | NO: 131) | | GGACAACGCAA (SEQ ID NO: 51) |
| Serpin E1 | MQMSPALTCLVLGL | P05121 | ATGCAAATGTCTCCTGCCCTTACCTG |
| | ALVFGEGSA (SEQ | | TCTCGTACTTGGTCTTGCGCTCGTAT |
| | ID NO: 132) | | TTGGAGAGGGATCAGCC (SEQ ID NO: |
| | | | 52) |
| GROalpha | MARAALSAAPSNPR | P09341 | ATGGCAAGGGCTGCACTCAGTGCTG |
| | LLRVALLLLLLVAA | | CCCCGTCTAATCCCAGATTGCTTCGA |
| | GRRAAG (SEQ ID | | GTTGCATTGCTTCTTCTGTTGCTGGT |
| | NO: 133) | | TGCAGCTGGTAGGAGAGCAGCGGGT |
| | | | (SEQ ID NO: 53) |
| CXCL12 | MNAKVVVVLVLVL | P48061 | ATGAATGCAAAAGTCGTGGTCGTGC |
| | TALCLSDG (SEQ ID | | TGGTTTTGGTTCTGACGGCGTTGTGT |
| | NO: 134) | | CTTAGTGATGGG (SEQ ID NO: 54) |
| IL-21 (Codon | MERIVICLMVIFLGT | Q9HBE4 | ATGGAACGCATTGTGATCTGCCTGAT |
| Optimized) | LVHKSSS (SEQ ID | | GGTCATCTTCCTGGGCACCTTAGTGC |
| | NO: 135) | | ACAAGTCGAGCAGC (SEQ ID NO: 55) |
| CD8 | MALPVTALLLPLAL | — | ATGGCCTTACCAGTGACCGCCTTGCT |
| | LLHAARP (SEQ ID | | CCTGCCGCTGGCCTTGCTGCTCCACG |
| | NO: 136) | | CCGCCAGGCCG (SEQ ID NO: 139) |
| CD8 (Codon | MALPVTALLLPLAL | — | ATGGCGCTCCCGGTGACAGCACTTCT |
| Optimized) | LLHAARP (SEQ ID | | CTTGCCTCTTGCCCTGCTGTTGCATG |
| | NO: 137) | | CCGCGCGCCCA (SEQ ID NO: 140) |
| | | | OR |
| | | | ATGGCTCTGCCCGTGACAGCTTTGCT |
| | | | GCTGCCTTTGGCACTGCTGCTGCATG |
| | | | CTGCTAGACCA (SEQ ID NO: 416) |
| GMCSF | MLLVTSLLLCELPHP | — | ATGTTGCTCGTGACATCCCTCTTGCT |
| | AFLLIP (SEQ ID NO: | | TTGTGAGTTGCCTCATCCCGCATTCC |
| | 138) | | TGCTCATCCCA (SEQ ID NO: 141) |
| NKG2D | PFFFCCFIAVAMGIR | | CCCTTCTTCTTCTGTTGCTTTATCGCC |
| | FIIMVA (SEQ ID NO: | | GTGGCCATGGGCATCCGCTTCATCAT |
| | 192) | | TATGGTGGCC (SEQ ID NO: 193) |
| IgE | MDWTWILFLVAAA | | ATGGACTGGACTTGGATACTCTTTCT |
| | TRVHS (SEQ ID NO: | | GGTCGCTGCCGCCACACGGGTGCAC |
| | 228) | | TCT (SEQ ID NO: 229) |
| GM-CSFRα | MLLLVTSLLLCELPH | | ATGCTGCTGCTGGTTACATCTCTGCT |
| | PAFLLIP (SEQ ID | | GCTGTGCGAGCTGCCCCATCCTGCCT |
| | 423). | | TTCTGCTTATTCCT (SEQ ID NO: 424) |

### Protease Cleavage Site

In certain embodiments, the chimeric proteins provided for herein (e.g., in general, for all membrane-cleavable chimeric proteins described herein) contain a protease cleavage site (e.g., referred to as “C” in the formula S—C-MT or MT-C—S for membrane-cleavable chimeric proteins described herein). In general, the protease cleavage site can be any amino acid sequence motif capable of being cleaved by a protease. Examples of protease cleavage sites include, but are not limited to, a Type 1 transmembrane protease cleavage site, a Type II transmembrane protease cleavage site, a GPI anchored protease cleavage site, an ADAM8 protease cleavage site, an ADAM9 protease cleavage site, an ADAM10 protease cleavage site, an ADAM12 protease cleavage site, an ADAM15 protease cleavage site, an ADAM17 protease cleavage site, an ADAM19 protease cleavage site, an ADAM20 protease cleavage site, an ADAM21 protease cleavage site, an ADAM28 protease cleavage site, an ADAM30 protease cleavage site, an ADAM33 protease cleavage site, a BACE1 protease cleavage site, a BACE2 protease cleavage site, a SIP protease cleavage site, an MT1-MMP protease cleavage site, an MT3-MMP protease cleavage site, an MT5-MMP protease cleavage site, a furin protease cleavage site, a PCSK7 protease cleavage site, a matriptase protease cleavage site, a matriptase-2 protease cleavage site, an MMP9 protease cleavage site, or an NS3 protease cleavage site.

One example of a protease cleavage site is a hepatitis C virus (HCV) nonstructural protein 3 (NS3) protease cleavage site, including, but not limited to, a NS3/NS4A, a NS4A/NS4B, a NS4B/NS5A, or a NS5A/NS5B cleavage site. For a description of NS3 protease and representative sequences of its cleavage sites for various strains of HCV, see, e.g., Hepatitis C Viruses: Genomes and Molecular Biology (S. L. Tan ed., Taylor & Francis, 2006), Chapter 6, pp. 163-206; herein incorporated by reference in its entirety. For example, the sequences of HCV NS4A/4B protease cleavage site; HCV NS5A/5B protease cleavage site; C-terminal degron with NS4A/4B protease cleavage site; N-terminal degron with HCV NS5A/5B protease cleavage site are provided. Representative NS3 sequences are listed in the National Center for Biotechnology Information (NCBI) database. See, for example, NCBI entries: Accession Nos. YP_001491553, YP_001469631, YP_001469632, NP_803144, NP_671491, YP_001469634, YP_001469630, YP_001469633, ADA68311, ADA68307, AFP99000, AFP98987, ADA68322, AFP99033, ADA68330, AFP99056, AFP99041, CBF60982, CBF60817, AHH29575, AIZ00747, AIZ00744, ABI36969, ABN05226, KF516075, KF516074, KF516056, AB826684, AB826683, JX171009, JX171008, JX171000, EU847455, EF154714, GU085487, JX171065, JX171063; all of which sequences (as entered by the date of filing of this application) are herein incorporated by reference.

Another example of a protease cleavage site is an ADAM17-specific protease (also referred to as Tumor Necrosis Factor-α Converting Enzyme [TACE]) cleavage site. An ADAM17-specific protease cleavage site can be an endogenous sequence of a substrate naturally cleaved by ADAM17. An ADAM17-specific protease cleavage site can be an engineered sequence capable of being cleaved by ADAM17. An engineered ADAM17-specific protease cleavage site can be an engineered for specific desired properties including, but not limited to, optimal expression of the chimeric proteins, specificity for ADAM17, rate-of-cleavage by ADAM17, ratio of secreted and membrane-bound chimeric protein levels, and cleavage in different cell states. A protease cleavage site can be selected for specific cleavage by ADAM17. For example, certain protease cleavage sites capable of being cleaved by ADAM17 are also capable of cleavage by additional ADAM family proteases, such as ADAM10. Accordingly, an ADAM17-specific protease cleavage site can be selected and/or engineered such that cleavage by other proteases, such as ADAM10, is reduced or eliminated. A protease cleavage site can be selected for rate-of-cleavage by ADAM17. For example, it can be desirable to select a protease cleavage site demonstrating a specific rate-of-cleavage by ADAM17, such as reduced cleavage kinetics relative to an endogenous sequence of a substrate naturally cleaved by ADAM17. In such cases, in general, a specific rate-of-cleavage can be selected to regulate the rate of processing of the chimeric protein, which in turn regulates the rate of release/secretion of the payload effector molecule. Accordingly, an ADAM17-specific protease cleavage site can be selected and/or engineered such that the sequence demonstrates a desired rate-of-cleavage by ADAM17. A protease cleavage site can be selected for both specific cleavage by ADAM17 and rate-of-cleavage by ADAM17. Exemplary ADAM17-specific protease cleavage sites, including those demonstrating particular specificity and rate-of-cleavage kinetics, are shown in Table 4A below with reference to the site of cleavage (P5-P1: N-terminal; P1′-P5′: C-terminal). Further details of ADAM17 and ADAM10, including expression and protease cleavage sites, are described in Sharma, et al. (J Immunol Oct. 15, 2017, 199 (8) 2865-2872), Pham et al. (Anticancer Res. 2017 October; 37 (10): 5507-5513), Caescu et al. (Biochem J. 2009 Oct. 23; 424 (1): 79-88), and Tucher et al. (J. Proteome Res. 2014, 13, 4, 2205-2214), each herein incorporated by reference for purposes.

TABLE 4A
Various ADAM17 Protease Cleavage
Site Sequences

| | | | | | | | | | | | SEQ ID |
|---|---|---|---|---|---|---|---|---|---|---|---|
| P5 | P4 | P3 | P2 | P1 | P1′ | P2′ | P3′ | P4′ | P5′ | FULL SEQ | NO |
| P | R | A | E | A | V | K | G | G | | PRAEAVKGG | 179 |
| P | R | A | E | A | L | K | G | G | | PRAEALKGG | 180 |
| P | R | A | E | Y | S | K | G | G | | PRAEYSKGG | 181 |
| P | R | A | E | P | I | K | G | G | | PRAEPIKGG | 182 |
| P | R | A | E | A | Y | K | G | G | | PRAEAYKGG | 183 |
| P | R | A | E | S | S | K | G | G | | PRAESSKGG | 184 |
| P | R | A | E | F | T | K | G | G | | PRAEFTKGG | 185 |
| D | E | P | H | Y | S | Q | R | R | | DEPHYSQRR | 187 |
| P | P | L | G | P | I | F | N | P | G | PPLGPIFNPG | 188 |
| P | L | A | Q | A | Y | R | S | S | | PLAQAYRSS | 189 |
| T | P | I | D | S | S | F | N | P | D | TPIDSSFNPD | 190 |
| V | T | P | E | P | I | F | S | L | I | VTPEPIFSLI | 191 |

In some embodiments, the protease cleavage site comprises a first region having the amino acid sequence of PRAE (SEQ ID NO: 176). In some embodiments, the protease cleavage site comprises a second region having the amino acid sequence of KGG (SEQ ID NO: 177). In some embodiments, the first region is located N-terminal to the second region. In some embodiments, the protease cleavage site comprises the amino acid sequence of PRAEX₁X₂KGG (SEQ ID NO: 178), wherein X₁ is A, Y, P, S, or F, and wherein X₂ is V, L, S, I, Y, T, or A. In some embodiments, the protease cleavage site comprises the amino acid sequence of PRAEX₁X₂KGG (SEQ ID NO: 178), wherein X₁ is A, Y, P, S, or F, and wherein X₂ is V, L, S, I, Y, or T. In some embodiments, the protease cleavage site comprises the amino acid sequence of PRAEAVKGG (SEQ ID NO: 179). In some embodiments, the protease cleavage site comprises the amino acid sequence of PRAEALKGG (SEQ ID NO: 180). In some embodiments, the protease cleavage site comprises the amino acid sequence of PRAEYSKGG (SEQ ID NO: 181). In some embodiments, the protease cleavage site comprises the amino acid sequence of PRAEPIKGG (SEQ ID NO: 182). In some embodiments, the protease cleavage site comprises the amino acid sequence of PRAEAYKGG (SEQ ID NO: 183). In some embodiments, the protease cleavage site comprises the amino acid sequence of PRAESSKGG (SEQ ID NO: 184). In some embodiments, the protease cleavage site comprises the amino acid sequence of PRAEFTKGG (SEQ ID NO: 185). In some embodiments, the protease cleavage site comprises the amino acid sequence of PRAEAAKGG (SEQ ID NO: 186).

In some embodiments, the protease cleavage site comprises the amino acid sequence of DEPHYSQRR (SEQ ID NO: 187). In some embodiments, the protease cleavage site comprises the amino acid sequence of PPLGPIFNPG (SEQ ID NO: 188). In some embodiments, the protease cleavage site comprises the amino acid sequence of PLAQAYRSS (SEQ ID NO: 189). In some embodiments, the protease cleavage site comprises the amino acid sequence of TPIDSSFNPD (SEQ ID NO: 190). In some embodiments, the protease cleavage site comprises the amino acid sequence of VTPEPIFSLI (SEQ ID NO: 191). The protease cleavage sites of SEQ ID NOs: 187, 189, and 191 are cleavable by ADAM17.

In some embodiments, the protease cleavage site can include an N-terminal peptide linker, such as SGGGGSGGGGSG (SEQ ID NO: 230). In some embodiments, the protease cleavage site can include a C-terminal peptide linker, such as GGGSGGGGSGGGSLQ (SEQ ID NO: 231). In some embodiments, the protease cleavage site can include an N-terminal peptide linker and a C-terminal peptide linker, such as both table (SEQ ID NO: 230) and GGGSGGGGSGGGSLQ (SEQ ID NO: 231).

In some embodiments, the protease cleavage site can include a Tumor Necrosis Factor-α Converting Enzyme (TACE)-specific cleavage site (also referred to as ADAM17), an N-terminal peptide linker, and a C-terminal peptide linker. In some embodiments, the protease cleavage site can include the TACE-specific cleavage site VTPEPIFSLI (SEQ ID NO: 191), an N-terminal peptide linker, and a C-terminal peptide linker. In some embodiments, the protease cleavage site can include the TACE-specific cleavage site, an N-terminal peptide linker, and a C-terminal peptide linker and have the amino acid sequence SGGGGSGGGGSGVTPEPIFSLIGGGSGGGGSGGGSLQ (SEQ ID NO: 250). In some embodiments, the protease cleavage site can include the TACE-specific cleavage site, an N-terminal peptide linker, and a C-terminal peptide linker encoded by a polynucleotide sequence comprising the sequence TCAGGCGGCGGTGGTAGTGGAGGCGGAGGCTCAGGCGTGACCCCTGAGCCTATCTT CAGCCTGATCGGCGGAGGTTCCGGAGGTGGCGGTTCCGGCGGAGGATCTCTTCAA (SEQ ID NO: 251). In some embodiments, the protease cleavage site can include the TACE-specific cleavage site, an N-terminal peptide linker, and a C-terminal peptide linker encoded by a polynucleotide sequence that is at least 90%, at least 91%, at least 92%, at least 93%, at least 94%, at least 95%, at least 96%, at least 97%, at least 98%, or at least 99% identical to

(SEQ ID NO: 251)
TCAGGCGGCGGTGGTAGTGGAGGCGGAGGCTCAGGCGTGACCCCT
GAGCCTATCTTCAGCCTGATCGGCGGAGGTTCCGGAGGTGGCGGT
TCCGGCGGAGGATCTCTTCAA.

In some embodiments, the protease cleavage site comprises the amino acid sequence of ITQGLAVSTISSFF (SEQ ID NO: 198), which is a cleavage site that is native to CD16 and is cleavable by ADAM17.

The protease cleavage site can be C-terminal of the secretable effector molecule. The protease cleavage site can be N-terminal of the secretable effector molecule. In general, for all membrane-cleavable chimeric proteins described herein, the protease cleavage site is either: (1) C-terminal of the secretable effector molecule and N-terminal of the cell membrane tethering domain (in other words, the protease cleavage site is in between the secretable effector molecule and the cell membrane tethering domain); or (2)N-terminal of the secretable effector molecule and C-terminal of the cell membrane tethering domain (also between the secretable effector molecule and the cell membrane tethering domain with domain orientation inverted). The protease cleavage site can be connected to the secretable effector molecule by a polypeptide linker, i.e., a polypeptide sequence not generally considered to be part of the effector molecule or protease cleavage site. The protease cleavage site can be connected to the cell membrane tethering domain by a polypeptide linker, i.e., a polypeptide sequence not generally considered to be part of the cell membrane tethering domain or protease cleavage site. A polypeptide linker can be any amino acid sequence that connects a first polypeptide sequence and a second polypeptide sequence. A polypeptide linker can be a flexible linker (e.g., a Gly-Ser-Gly sequence). Examples of polypeptide linkers include, but are not limited to, GSG linkers (e.g., [GS]₄GG [SEQ ID NO: 504]), A(EAAAK)₃A (SEQ ID NO: 505), and Whitlow linkers (e.g., a “KEGS” (SEQ ID NO: 506) linker such as the amino acid sequence KESGSVSSEQLAQFRSLD (SEQ ID NO: 184), an eGK linker such as the amino acid sequence EGKSSGSGSESKST (SEQ ID NO: 185), and linkers described in more detail in Issued U.S. Pat. No. 5,990,275 herein incorporated by reference). Additional exemplary polypeptide linkers include SEQ ID NO: 194, SEQ ID NO: 195, SEQ ID NO: 196, and SEQ ID NO: 197. Other polypeptide linkers may be selected based on desired properties (e.g., length, flexibility, amino acid composition etc.) and are known to those skilled in the art.

In the Membrane-Cleavable system, following expression and localization of the chimeric protein into the cell membrane, the protease cleavage site directs cleavage of the chimeric protein such that the effector molecule is released (“secreted”) into the extracellular space of a cell.

In general, a protease that cleaves the protease cleavage site is a protease specific for that specific protease cleavage site. For example, in the case of a disintegrin and metalloproteinase (“ADAM”) family protease, the protease that cleaves a specific ADAM protease cleavage site is generally limited to the ADAM protease(s) that specifically recognize the specific ADAM protease cleavage site motif. A protease cleavage site can be selected and/or engineered such that cleavage by undesired proteases is reduced or eliminated. Proteases can be membrane-bound or membrane-associated. Proteases can be secreted, e.g., secreted in a specific cellular environment, such as a tumor microenvironment (“TME”).

A protease that cleaves the protease cleavage site of the chimeric protein can be expressed in the same cell that expresses the chimeric protein. A protease that cleaves the protease cleavage site of the chimeric protein can be endogenous to a cell expressing the chimeric protein. In other words, a cell engineered to express the chimeric protein can endogenously express the protease specific for the protease cleavage site present in the chimeric protein. Endogenous expression of the protease refers to both expression under generally homeostatic conditions (e.g., a cell generally considered to be healthy), and also to differential expression under non-homeostatic conditions (e.g., upregulated expression in a tumor cell). The protease cleavage site can be selected based on the known proteases endogenously expressed by a desired cell population. In such cases, in general, the cleavage of the protease cleavage site (and thus release/secretion of a payload) can be restricted to only those cells of interest due to the cell-restricted protease needing to come in contact with the protease cleavage site of chimeric protein expressed in the same cell. For example, and without wishing to be bound by theory, ADAM17 is believed to be restricted in its endogenous expression to NK cell and T cells. Thus, selection of an ADAM17-specific protease cleavage site may restrict the cleavage of the protease cleavage site to NK cell and T cells co-expressing the chimeric protein. In other examples, a protease cleavage site can be selected for a specific tumor-associated protease known to be expressed in a particular tumor population of interest (e.g., in a specific tumor cell engineered to express the chimeric protein). Protease and/or expression databases can be used to select an appropriate protease cleavage site, such as selecting a protease cleavage site cleaved by a tumor-associated proteases through consulting Oncomine (www.oncominc.org), the European Bioinformatic Institute (www.cbi.ac.uk) in particular (www.cbi.ac.uk/gxa), PMAP (www.proteolysis.org), ExPASy Peptide Cutter (ca.expasy.org/tools/peptide cutter) and PMAP.Cut DB (cutdb.burnham.org), each of which is incorporated by reference for all purposes.

A protease that cleaves the protease cleavage site of the chimeric protein can be heterologous to a cell expressing the chimeric protein. For example, a cell engineered to express the chimeric protein can also be engineered to express a protease not generally expressed by the cell that is specific for the protease cleavage site present in the chimeric protein. A cell engineered to express both the chimeric protein and the protease can be engineered to express each from separate engineered nucleic acids or from a multicistronic systems (multicistronic and multi-promoter systems are described in greater detail in the Section herein titled “Multicistronic and Multiple Promoter Systems”). Heterologous proteases and their corresponding protease cleavage site can be selected as described above with reference to endogenous proteases.

A protease that cleaves the protease cleavage site of the chimeric protein can be expressed on a separate distinct cell than the cell that expresses the chimeric protein. For example, the protease can be generally expressed in a specific cellular environment, such as a tumor microenvironment. In such cases, in general, the cleavage of the protease cleavage site can be restricted to only those cellular environments of interest (e.g., a tumor microenvironment) due to the environment-restricted protease needing to come in contact with the protease cleavage site. In embodiments having membrane-cleavable chimeric proteins, in general, the secretion of the effector molecule can be restricted to only those cellular environments of interest (e.g., a tumor microenvironment) due to the environment-restricted protease needing to come in contact with the protease cleavage site. A protease that cleaves the protease cleavage site of the chimeric protein can be endogenous to the separate distinct cell. A protease that cleaves the protease cleavage site of the chimeric protein can be heterologous to the separate distinct cell. For example, the separate distinct cell can be engineered to express a protease not generally expressed by the separate distinct cell.

Proteases include, but are not limited to, a Type 1 transmembrane protease, a Type II transmembrane protease, a GPI anchored protease, an ADAM8 protease, an ADAM9 protease, an ADAM10 protease, an ADAM12 protease, an ADAM15 protease, an ADAM17 protease, an ADAM19 protease, an ADAM20 protease, an ADAM21 protease, an ADAM28 protease, an ADAM30 protease, an ADAM33 protease, a BACE1 protease, a BACE2 protease, a SIP protease, an MT1-MMP protease, an MT3-MMP protease, an MT5-MMP protease, a furin protease, a PCSK7 protease, a matriptase protease, a matriptase-2 protease, and an MMP9 protease. A protease can be an NS3 protease. A protease can be an ADAM17 protease.

Proteases can be tumor associated proteases, such as, a cathepsin, a cysteine protease, an aspartyl protease, a serine protease, or a metalloprotease. Specific examples of tumor associated proteases include Cathepsin B, Cathepsin L, Cathepsin S, Cathepsin D, Cathepsin E, Cathepsin A, Cathepsin G, Thrombin, Plasmin, Urokinase, Tissue Plasminogen Activator, Metalloproteinas 1 (MMP1), MMP2, MMP3, MMP4, MMP7, MMP8, MMP9, MMP10, MMP11, MMP12, MMP13, MMP14, MMP15, MMP16, MMP17, MMP20, MMP21, MMP23, MMP24, MMP25, MMP26, MMP28, ADAM, ADAMTS, CD10 (CALLA), or prostate specific antigen. Proteases can also include, but are not limited to, proteases listed in Table 4B below. Exemplary cognate protease cleavage sites for certain proteases are also listed in Table 4B.

TABLE 4B
Exemplary Proteases with Cognate Cleavage Sites and Inhibitors

| Protease | | |
|---|---|---|
| (UniProt Accession No.) | Cognate cleavage site | Protease inhibitors |
| HCV NS4A/4B | DEMEECSQHL | Simeprevir, Danoprevir, |
| | (SEQ ID NO: 142) | Asunaprevir, Ciluprevir, |
| | EDVVPCSMG | Boceprevir, Sovaprevir, |
| | (SEQ ID NO: 143) | Paritaprevir, Telaprevir, |
| | | Grazoprevir |
| HCV NS5A/5B | DEMEECSQHL | Simeprevir, Danoprevir, |
| | (SEQ ID NO: 142) | Asunaprevir, Ciluprevir, |
| | EDVVPCSMG | Boceprevir, Sovaprevir, |
| | (SEQ ID NO: 143) | Paritaprevir, Telaprevir, |
| | | Grazoprevir |
| HCV NS3 | DEMEECSQHL | Simeprevir, Danoprevir, |
| | (SEQ ID NO: 142) | Asunaprevir, Ciluprevir, |
| | EDVVPCSMG | Boceprevir, Sovaprevir, |
| | (SEQ ID NO: 143) | Paritaprevir, Telaprevir, |
| | | Grazoprevir |
| HCV NS2-3 | DEMEECSQHL | Simeprevir, Danoprevir, |
| | (SEQ ID NO: 142) | Asunaprevir, Ciluprevir, |
| | EDVVPCSMG | Boceprevir, Sovaprevir, |
| | (SEQ ID NO: 143) | Paritaprevir, Telaprevir, |
| | | Grazoprevir |
| HIV-1 protease | | Amprenavir, Atazanavir, |
| (SEQ ID NO: 144) | | Darunavir, |
| | | Fosamprenavir, |
| | | Indinavir, Lopinavir, |
| | | Nelfinavir, Ritonavir, |
| | | Saquinavir, Tipranavir |
| Signal peptidase (P67812, | preference of eukaryotic signal | |
| P15367, P00804, P0803) | peptidase for cleavage after residue | |
| | 20 (Xaa^{20↓}) of pre(Δpro)apoA-II: | |
| | Ala, Cys > Gly > Ser, Thr > Pro > | |
| | Asn, Val, Ile, Leu, Tyr, His, Arg, | |
| | Asp. | |
| proprotein convertases | (R/K)-X-(hydrophobic)-X↓, where | |
| cleaving at hydrophobic | X is any amino acid | |
| residues (e.g., Leu, Phe, | | |
| Val, or Met) (Q16549, | | |
| Q8NBP7, Q92824, | | |
| P29120, Q6UW60, | | |
| P29122, Q9QXV0) | | |
| proprotein convertases | (K/R)-(X)n-(K/R)↓, where n is 0, 2, | |
| cleaving at small amino | 4 or 6 and X is any amino acid | |
| acid residues such as Ala | | |
| or Thr (Q16549, | | |
| Q8NBP7, Q92824, | | |
| P29120, Q6UW60, | | |
| P29122) | | |
| proopiomelanocortin | Cleavage at paired basic residues in | |
| converting enzyme (PCE) | certain prohormones, either between | |
| (Q9UO77615, O776133) | them, or on the carboxyl side | |
| chromaffin granule | tends to cleave dipeptide bonds that | |
| aspartic protease (CGAP) | have hydrophobic residues as well | |
| | as a beta-methylene group | |
| prohormone thiol protease | | |
| (cathepsin L1) (P07154, | | |
| P07711, P06797, P25975, | | |
| Q28944) | | |
| carboxypeptidases (e.g., | cleaves a peptide bond at the | |
| carboxypeptidase E/H, | carboxy-terminal (C-terminal) end | |
| carboxypeptidase D and | of a protein or peptide | |
| carboxypeptidase Z) | | |
| (Q9M099, P15169, | | |
| Q04609, P08819, P08818, | | |
| O77564, P70627, | | |
| O35409, P07519, | | |
| Q8VZU3, P22792, | | |
| P15087, P16870, | | |
| Q9JHH6, Q96IY4, | | |
| Q7L8A9) | | |
| aminopeptidases (e.g., | cleaves a peptide bond at the amino- | |
| arginine aminopeptidase, | terminal (N-terminal) end of a | |
| lysine aminopeptidase, | protein or peptide | |
| aminopeptidase B) | | |
| prolyl endopeptidase | Hydrolysis of Pro-\|-Xaa >> Ala-\|- | |
| (Q12884, P48147, | Xaa in oligopeptides. | |
| P97321, Q4J6C6) | Release of an N-terminal dipeptide, | |
| | Xaa-Yaa-\|-Zaa-, from a polypeptide, | |
| | preferentially when Yaa is Pro, | |
| | provided Zaa is neither Pro nor | |
| | hydroxyproline | |
| aminopeptidase N | Release of an N-terminal amino | |
| (P97449, P15144, | acid, Xaa-\|-Yaa- from a peptide, | |
| P15145, P15684) | amide or arylamide. Xaa is | |
| | preferably Ala, but may be most | |
| | amino acids including Pro (slow | |
| | action). When a terminal | |
| | hydrophobic residue is followed by | |
| | a prolyl residue, the two may be | |
| | released as an intact Xaa-Pro | |
| | dipeptide | |
| insulin degrading enzyme | Degradation of insulin, glucagon | |
| (P14735, P35559, | and other polypeptides. No action on | |
| Q9JHR7, P22817, | proteins. | |
| Q24K02) | Cleaves multiple short polypeptides | |
| | that vary considerably in sequence | |
| Calpain (O08529, | No specific amino acid sequence is | |
| P17655, Q07009, | uniquely recognized by calpains. | |
| Q27971, P20807, P07384, | Amongst protein substrates, tertiary | |
| O35350, O14815, | structure elements rather than | |
| P04632, Q9Y6Q1, | primary amino acid sequences | |
| O15484, Q9HC96, | appear to be responsible for | |
| A6NHC0, Q9UMQ6) | directing cleavage to a specific | |
| | substrate. Amongst peptide and | |
| | small-molecule substrates, the most | |
| | consistently reported specificity is | |
| | for small, hydrophobic amino acids | |
| | (e.g., leucine, valine and isoleucine) | |
| | at the P2 position, and large | |
| | hydrophobic amino acids (e.g., | |
| | phenylalanine and tyrosine) at the | |
| | P1 position. One fluorogenic calpain | |
| | substrate is (EDANS)-Glu-Pro-Leu- | |
| | Phe=Ala-Glu-Arg-Lys-(SEQ ID | |
| | NO: 507) (DABCYL), | |
| | (EDANSEPLFAERKDABCYL | |
| | (SEQ ID NO: 145)) with cleavage | |
| | occurring at the Phe=Ala bond. | |
| caspase 1 (P29466, | Strict requirement for an Asp | |
| P29452) | residue at position P1 and has a | |
| | preferred cleavage sequence of Tyr- | |
| | Val-Ala-Asp-\|- (YVAD; SEQ ID | |
| | NO: 146). | |
| caspase 2 (P42575, | Strict requirement for an Asp | |
| P29594) | residue at P1, with 316-asp being | |
| | essential for proteolytic activity and | |
| | has a preferred cleavage sequence of | |
| | Val-Asp-Val-Ala-Asp-\|- (VDVAD; | |
| | SEQ ID NO: 147). | |
| caspase 3 (P42574, | Strict requirement for an Asp | |
| P70677) | residue at positions P1 and P4. It has | |
| | a preferred cleavage sequence of | |
| | Asp-Xaa-Xaa-Asp-\|- with a | |
| | hydrophobic amino-acid residue at | |
| | P2 and a hydrophilic amino-acid | |
| | residue at P3, although Val or Ala | |
| | are also accepted at this position. | |
| caspase 4 (P70343, | Strict requirement for Asp at the P1 | |
| P49662) | position. It has a preferred cleavage | |
| | sequence of Tyr-Val-Ala-Asp-\|- | |
| | (YVAD; SEQ ID NO: 146) but also | |
| | cleaves at Asp-Glu-Val-Asp-\|- | |
| | (DEVD; SEQ ID NO: 148). | |
| caspase 5 (P51878) | Strict requirement for Asp at the P1 | |
| | position. It has a preferred cleavage | |
| | sequence of Tyr-Val-Ala-Asp-I- | |
| | (YVAD; SEQ ID NO: 146) but also | |
| | cleaves at Asp-Glu-Val-Asp-\|- -\|- | |
| | (DEVD; SEQ ID NO: 148). | |
| caspase 6 (P55212) | Strict requirement for Asp at | |
| | position P1 and has a preferred | |
| | cleavage sequence of Val-Glu-His- | |
| | Asp-\|-(VEHD; SEQ ID NO: 149). | |
| caspase 7 (P97864, | Strict requirement for an Asp | |
| P55210) | residue at position P1 and has a | |
| | preferred cleavage sequence of Asp- | |
| | Glu-Val-Asp-\|- (DEVD; SEQ ID | |
| | NO: 148). | |
| caspase 8 (Q8IRY7, | Strict requirement for Asp at | |
| O89110, Q14790) | position P1 and has a preferred | |
| | cleavage sequence of | |
| | (Leu/Asp/Val)-Glu-Thr-Asp-\|- | |
| | (Gly/Ser/Ala). | |
| caspase 9 (P55211, | Strict requirement for an Asp | |
| Q8C3Q9, Q5IS54) | residue at position P1 and with a | |
| | marked preference for His at | |
| | position P2. It has a preferred | |
| | cleavage sequence of Leu-Gly-His- | |
| | Asp-\|-Xaa (LGHD; SEQ ID NO: | |
| | 150). | |
| caspase 10 (Q92851) | Strict requirement for Asp at | |
| | position P1 and has a preferred | |
| | cleavage sequence of Leu-Gln-Thr- | |
| | Asp-\|-Gly (LQTDG; SEQ ID NO: | |
| | 151). | |
| puromycin sensitive | Release of an N-terminal amino | |
| aminopeptidase (P55786, | acid, preferentially alanine, from a | |
| Q11011) | wide range of peptides, amides and | |
| | arylamides. | |
| angiotensin converting | Release of a C-terminal dipeptide, | Benazepril (Lotensin), |
| enzyme (ACE) (P12821, | oligopeptide-\|-Xaa-Yaa, when Xaa | Captopril, Enalapril |
| P09470, Q9BYF1) | is not Pro, and Yaa is neither Asp | (Vasotec), Fosinopril, |
| SEQ ID NO: 156 | nor Glu. | Lisinopril (Prinivil, |
| | | Zestril), Moexipril, |
| | | Perindopril (Aceon), |
| | | Quinapril (Accupril), |
| | | Ramipril (Altace), |
| | | Trandolapril (Mavik), |
| | | Zofenopril |
| pyroglutamyl peptidase II | Release of the N-terminal | |
| (Q9NXJ5) | pyroglutamyl group from pGlu -- His- | |
| | Xaa tripeptides and pGlu -- His-Xaa- | |
| | Gly tetrapeptides | |
| dipeptidyl peptidase IV | Release of an N-terminal dipeptide, | |
| (P27487, P14740, | Xaa-Yaa-\|-Zaa-, from a polypeptide, | |
| P28843) | preferentially when Yaa is Pro, | |
| | provided Zaa is neither Pro nor | |
| | hydroxyproline | |
| N-arginine dibasic | Hydrolysis of polypeptides, | |
| convertase (O43847, | preferably at -Xaa-\|-Arg-Lys-, and | |
| Q8BHG1) | less commonly at -Arg-\|-Arg-Xaa-, | |
| | in which Xaa is not Arg or Lys | |
| endopeptidase 24.15 | Preferential cleavage of bonds with | |
| (thimet oligopeptidase) | hydrophobic residues at P1, P2 and | |
| (P52888, P24155) | P3′ and a small residue at P1′ in | |
| | substrates of 5 to 15 residues | |
| endopeptidase 24.16 | Preferential cleavage in neurotensin: | |
| (neurolysin) (Q9BYT8, | 10-Pro-\|-Tyr-11 | |
| Q91YP2) | | |
| amyloid precursor protein | Endopeptidase of broad specificity. | |
| secretase alpha (P05067, | | |
| P12023, Q9Y5Z0, | | |
| P56817) | | |
| amyloid precursor protein | Broad endopeptidase specificity. | |
| secretase beta (P05067, | Cleaves Glu-Val-Asn-Leu-\|-Asp- | |
| P12023, Q9Y5Z0, | Ala-Glu-Phe (EVNLDAEF; SEQ ID | |
| P56817) | NO: 152) in the Swedish variant of | |
| | Alzheimer's amyloid precursor | |
| | protein | |
| amyloid precursor protein | intramembrane cleavage of integral | |
| secretase gamma (P05067, | membrane proteins | |
| P12023, Q9Y5Z0, | | |
| P56817) | | |
| MMP 1 (P03956, | Cleavage of the triple helix of | SB-3CT |
| Q9EPL5uy) | collagen at about three-quarters of | p-OH SB-3CT |
| | the length of the molecule from the | O-phosphate SB-3CT |
| | N-terminus, at 775-Gly-\|-Ile-776 in | RXP470.1 |
| | the alpha-1(I) chain. Cleaves | |
| | synthetic substrates and alpha- | |
| | macroglobulins at bonds where P1′ | |
| | is a hydrophobic residue. | |
| MMP 2 (P08253, P33434) | Cleavage of gelatin type I and | SB-3CT |
| | collagen types IV, V, VII, X. | p-OH SB-3CT |
| | Cleaves the collagen-like sequence | O-phosphate SB-3CT |
| | Pro-Gln-Gly-\|-Ile-Ala-Gly-Gln | RXP470.1 |
| | (PQGIAGQ; SEQ ID NO: 153). | |
| MMP 3 (P08254, P28862) | Preferential cleavage where P1′, P2′ | SB-3CT |
| | and P3′ are hydrophobic residues. | p-OH SB-3CT |
| | | O-phosphate SB-3CT |
| | | RXP470.1 |
| MMP 7 (P09237, | Cleavage of 14-Ala-\|-Leu-15 and | SB-3CT |
| Q10738) | 16-Tyr-\|-Leu-17 in B chain of | p-OH SB-3CT |
| | insulin. No action on collagen types | O-phosphate SB-3CT |
| | I, II, IV, V. Cleaves gelatin chain | RXP470.1 |
| | alpha-2(I) > alpha-1(I). | |
| MMP 8 (P22894, | Can degrade fibrillar type I, II, and | SB-3CT |
| O70138) | III collagens. | p-OH SB-3CT |
| | Cleavage of interstitial collagens in | O-phosphate SB-3CT |
| | the triple helical domain. Unlike EC | RXP470.1 |
| | 3.4.24.7, this enzyme cleaves type | |
| | III collagen more slowly than type I. | |
| MMP 9 (P14780, P41245) | Cleavage of gelatin types I and V | SB-3CT |
| | and collagen types IV and V. | p-OH SB-3CT |
| | Cleaves KiSS1 at a Gly-\|-Leu bond. | O-phosphate SB-3CT |
| | Cleaves type IV and type V collagen | RXP470.1 |
| | into large C-terminal three quarter | |
| | fragments and shorter N-terminal | |
| | one quarter fragments. Degrades | |
| | fibronectin but not laminin or Pz- | |
| | peptide. | |
| MMP 10 (P09238, | Can degrade fibronectin, gelatins of | SB-3CT |
| O55123) | type I, III, IV, and V; weakly | p-OH SB-3CT |
| | collagens III, IV, and V. | O-phosphate SB-3CT |
| | | RXP470.1 |
| MMP 11 (P24347, | A(A/Q)(N/A)↓(L/Y)(T/V/M/R)(R/K) | SB-3CT |
| Q02853) | G(G/A)EILR (SEQ ID NO: 508) | p-OH SB-3CT |
| | ↓ denotes the cleavage site | O-phosphate SB-3CT |
| | | RXP470.1 |
| MMP 12 (P39900, | Hydrolysis of soluble and insoluble | SB-3CT |
| P34960) | elastin. Specific cleavages are also | p-OH SB-3CT |
| | produced at 14-Ala-\|-Leu-15 and 16- | O-phosphate SB-3CT |
| | Tyr-\|-Leu-17 in the B chain of | RXP470.1 |
| | insulin | |
| | Has significant elastolytic activity. | |
| | Can accept large and small amino | |
| | acids at the Pl′ site, but has a | |
| | preference for leucine. Aromatic or | |
| | hydrophobic residues are preferred | |
| | at the P1 site, with small | |
| | hydrophobic residues (preferably | |
| | alanine) occupying P3 | |
| MMP 13 (P45452, | Cleaves triple helical collagens, | SB-3CT |
| P33435) | including type I, type II and type III | p-OH SB-3CT |
| | collagen, but has the highest activity | O-phosphate SB-3CT |
| | with soluble type II collagen. Can | RXP470.1 |
| | also degrade collagen type IV, type | |
| | XIV and type X | |
| MMP 14 (P50281, | Activates progelatinase A by | SB-3CT |
| P53690) | cleavage of the propeptide at 37- | p-OH SB-3CT |
| | Asn-\|-Leu-38. Other bonds | O-phosphate SB-3CT |
| | hydrolyzed include 35-Gly-\|-Ile-36 | RXP470.1 |
| | in the propeptide of collagenase 3, | |
| | and 341-Asn-\|-Phe-342, 441-Asp-\|- | |
| | Leu-442 and 354-Gln-\|-Thr-355 in | |
| | the aggrecan interglobular domain. | |
| urokinase plasminogen | Specific cleavage of Arg-\|-Val bond | Plasminogen activator |
| activator (uPA) (P00749, | in plasminogen to form plasmin. | inhibitors (PAI) |
| P06869) | | |
| tissue plasminogen | Specific cleavage of Arg-\|-Val bond | Plasminogen activator |
| activator (tPA) (P00750, | in plasminogen to form plasmin. | inhibitors (PAI) |
| P11214) | | |
| tissue plasminogen | Specific cleavage of Arg-\|-Val bond | Plasminogen activator |
| activator (tPA) (P00750, | in plasminogen to form plasmin. | inhibitors (PAI) |
| P11214) | | |
| Plasmin (P00747, | Preferential cleavage: Lys-\|-Xaa > | α-2-antiplasmin (AP) |
| P20918) | Arg-\|-Xaa, higher selectivity than | |
| | trypsin. Converts fibrin into soluble | |
| | products. | |
| Thrombin (P00734, | Cleaves bonds after Arg and Lys | |
| P19221) | Converts fibrinogen to fibrin and | |
| | activates factors V, VII, VIII, XIII, | |
| | and, in complex with | |
| | thrombomodulin, protein C. | |
| BMP-1 (procollagen C- | Cleavage of the C-terminal | |
| peptidase) (P13497, | propeptide at Ala-\|-Asp in type I and | |
| P98063) | II procollagens and at Arg-\|-Asp in | |
| | type III. | |
| ADAM (Q9POK1, | | SB-3CT |
| Q9UKQ2, Q9JLN6, | | p-OH SB-3CT |
| 014672, Q13444, | | O-phosphate SB-3CT |
| P78536, Q13443, | | RXP470.1 |
| O43184, P78325, | | |
| Q9UKF5, Q9BZ11, | | |
| Q9H2U9, Q99965, | | |
| O75077, Q9H013, | | |
| O43506) | | |
| granzyme A (P12544, | Preferential cleavage: - Arg-\|-Xaa-, | |
| P11032) | -Lys-\|-Xaa- >> -Phe-\|-Xaa- in small | |
| | molecule substrates. | |
| granzyme B (P10144, | Preference for bulky and aromatic | |
| P04187) | residues at the P1 position and acidic | |
| | residues at the P3′ and P4′ sites. | |
| granzyme M (P51124, | Cleaves peptide substrates after | |
| Q03238) | methionine, leucine, and norleucine. | |
| tobacco Etch virus (TEV) | E-Xaa-Xaa-Y -Xaa-Q-(G/S), with | |
| protease (P04517, | cleavage occurring between Q and | |
| POCK09) | G/S. The most common sequence is | |
| | ENLYFQS (SEQ ID NO: 154) | |
| chymotrypsin-like serine | | -Thermobifida fusca |
| protease (P08217, | | Thermopin |
| Q9UNII, Q91X79, | | -Pyrobaculum |
| P08861, P09093, P08218) | | aerophilum Aeropin |
| | | -Thermococcus |
| | | kodakaraensis Tk-serpin |
| | | -Alteromonas sp. |
| | | Marinostatin |
| | | -Streptomyces |
| | | misionensis SMTI |
| | | -Streptomyces sp. |
| | | chymostatin |
| alphavirus proteases | | |
| (P08411, P03317, | | |
| P13886, Q8JUX6, | | |
| Q86924, Q4QXJ8, | | |
| Q8QL53, P27282, | | |
| Q5XXP4) | | |
| chymotrypsin-like | | -Thermobifida fusca |
| cysteine proteases | | Thermopin |
| (Q86TL0, Q14790, | | -Pyrobaculum |
| Q99538, O15553) | | aerophilum Aeropin |
| | | -Thermococcus |
| | | kodakaraensis Tk-serpin |
| | | -Alteromonas sp. |
| | | Marinostatin |
| | | -Streptomyces |
| | | misionensis SMTI |
| | | -Streptomyces sp. |
| | | chymostatin |
| papain-like cysteine | | |
| proteases (P25774, | | |
| P53634, Q96K76) | | |
| picornavirus leader | | |
| proteases (P03305, | | |
| P03311, P13899) | | |
| HIV proteases (P04585, | | |
| P03367, P04584, P03369, | | |
| P12497, P03366, P04587) | | |
| Herpesvirus proteases | | |
| (P10220, Q2HRB6, | | |
| 040922, Q69527) | | |
| adenovirus proteases | | |
| (P03252, P24937, | | |
| Q83906, P68985, P09569, | | |
| P11825, P10381) | | |
| Streptomyces griseus | | |
| protease A (SGPA) | | |
| (P00776) | | |
| Streptomyces griseus | | |
| protease B (SGPB) | | |
| (P00777) | | |
| alpha-lytic protease | | |
| (P85142, P00778) | | |
| serine proteases (P48740, | | |
| P98064, Q9UL52, | | |
| P05981, 060235) | | |
| cysteine proteases | | |
| (Q86TL0, Q14790, | | |
| Q8WYNO, Q96DT6, | | |
| P55211) | | |
| aspartic proteases | | |
| (Q9Y5Z0, P56817, | | |
| Q00663, Q53RT3, | | |
| P0CY27) | | |
| threonine proteases | | |
| (Q9UI38, Q16512, | | |
| Q9H6P5, Q8IWU2) | | |
| Mast cell (MC) chymase | Abz-HPFHL(SEQ ID NO: 155)- | BAY 1142524 |
| (CMA1) (NM_001836) | Lys(Dnp)-NH2 | SUN13834 |
| Rat mast cell protease-1, | Abz-HPFHL(SEQ ID NO: 155)- | TY-51469 |
| -2, -3, -4, -5 (NM_017145, | Lys(Dnp)-NH2 | |
| NM_172044, | | |
| NM_001170466, | | |
| NM_019321, | | |
| NM_013092) | | |
| Rat vascular chymase | Abz-HPFHL(SEQ ID NO: 155)- | |
| (RVCH) (O70500) | Lys(Dnp)-NH2 | |
| DENV NS3pro | A strong preference for basic amino | Anthraquinone |
| (NS2B/NS3) | acid residues (Arg/Lys) at the P1 | BP13944 |
| SEQ ID NOs: 157, 158, | positions was observed, whereas the | ZINC04321905 |
| 159, 160 | preferences for the P2- 4 sites were | MB21 |
| | in the order of Arg > Thr > | Policresulen |
| | Gln/Asn/Lys for P2, Lys > Arg > | SK-12 |
| | Asn for P3, and Nle > Leu > Lys > | NSC135618 |
| | Xaa for P4. The prime site substrate | Biliverdin |
| | specificity was for small and polar | |
| | amino acids in P1 and P3. | |

A protease can be any of the following human proteases (MEROPS peptidase database number provided in parentheses; Rawlings N. D., Morton F. R., Kok, C. Y., Kong, J. & Barrett A. J. (2008) MEROPS: the peptidase database. Nucleic Acids Res. 36 Database issue, D320-325; herein incorporated by reference for all purposes): pepsin A (MER000885), gastricsin (MER000894), memapsin-2 (MER005870), renin (MER000917), cathepsin D (MER000911), cathepsin E (MER000944), memapsin-1 (MER005534), napsin A (MER004981), Mername-AA034 peptidase (MER014038), pepsin A4 (MER037290), pepsin A5 (Homo sapiens) (MER037291), hCG1733572 (Homo sapiens)-type putative peptidase (MER107386), napsin B pseudogenc (MER004982), CYMP g.p. (Homo sapiens) (MER002929), subfamily AlA unassigned peptidases (MER 181559), mouse mammary tumor virus retropepsin (MER048030), rabbit endogenous retrovirus endopeptidase (MER043650), S71-related human endogenous retropepsin (MER001812), RTVL-H-type putative peptidase (MER047117), RTVL-H-type putative peptidase (MER047133), RTVL-H-type putative peptidase (MER047160), RTVL-H-type putative peptidase (MER047206), RTVL-H-type putative peptidase (MER047253), RTVL-H-type putative peptidase (MER047260), RTVL-H-type putative peptidase (MER047291), RTVL-H-type putative peptidase (MER047418), RTVL-H-type putative peptidase (MER047440), RTVL-H-type putative peptidase (MER047479), RTVL-H-type putative peptidase (MER047559), RTVL-H-type putative peptidase (MER047583), RTVL-H-type putative peptidase (MER015446), human endogenous retrovirus retropepsin homologue 1 (MER015479), human endogenous retrovirus retropepsin homologue 2 (MER015481), endogenous retrovirus retropepsin pseudogene 1 (Homo sapiens chromosome 14) (MER029977), endogenous retrovirus retropepsin pseudogene 2 (Homo sapiens chromosome 8) (MER029665), endogenous retrovirus retropepsin pseudogene 3 (Homo sapiens chromosome 17) (MER002660), endogenous retrovirus retropepsin pseudogene 3 (Homo sapiens chromosome 17) (MER030286), endogenous retrovirus retropepsin pseudogene 3 (Homo sapiens chromosome 17) (MER047144), endogenous retrovirus retropepsin pseudogene 5 (Homo sapiens chromosome 12) (MER029664), endogenous retrovirus retropepsin pseudogene 6 (Homo sapiens chromosome 7) (MER002094), endogenous retrovirus retropepsin pseudogene 7 (Homo sapiens chromosome 6) (MER029776), endogenous retrovirus retropepsin pseudogene 8 (Homo sapiens chromosome Y) (MER030291), endogenous retrovirus retropepsin pseudogene 9 (Homo sapiens chromosome 19) (MER029680), endogenous retrovirus retropepsin pseudogene 10 (Homo sapiens chromosome 12) (MER002848), endogenous retrovirus retropepsin pseudogene 11 (Homo sapiens chromosome 17) (MER004378), endogenous retrovirus retropepsin pseudogene 12 (Homo sapiens chromosome 11) (MER003344), endogenous retrovirus retropepsin pseudogene 13 (Homo sapiens chromosome 2 and similar) (MER029779), endogenous retrovirus retropepsin pseudogene 14 (Homo sapiens chromosome 2) (MER029778), endogenous retrovirus retropepsin pseudogene 15 (Homo sapiens chromosome 4) (MER047158), endogenous retrovirus retropepsin pseudogene 15 (Homo sapiens chromosome 4) (MER047332), endogenous retrovirus retropepsin pseudogene 15 (Homo sapiens chromosome 4) (MER003182), endogenous retrovirus retropepsin pseudogene 16 (MER047165), endogenous retrovirus retropepsin pseudogene 16 (MER047178), endogenous retrovirus retropepsin pseudogene 16 (MER047200), endogenous retrovirus retropepsin pseudogene 16 (MER047315), endogenous retrovirus retropepsin pseudogene 16 (MER047405), endogenous retrovirus retropepsin pseudogene 16 (MER030292), endogenous retrovirus retropepsin pseudogene 17 (Homo sapiens chromosome 8) (MER005305), endogenous retrovirus retropepsin pseudogene 18 (Homo sapiens chromosome 4) (MER030288), endogenous retrovirus retropepsin pseudogene 19 (Homo sapiens chromosome 16) (MER001740), endogenous retrovirus retropepsin pseudogene 21 (Homo sapiens) (MER047222), endogenous retrovirus retropepsin pseudogene 21 (Homo sapiens) (MER047454), endogenous retrovirus retropepsin pseudogene 21 (Homo sapiens) (MER047477), endogenous retrovirus retropepsin pseudogene 21 (Homo sapiens) (MER004403), endogenous retrovirus retropepsin pseudogene 22 (Homo sapiens chromosome X) (MER030287), subfamily A2A non-peptidase homologues (MER047046), subfamily A2A non-peptidase homologues (MER047052), subfamily A2A non-peptidase homologues (MER047076), subfamily A2A non-peptidase homologues (MER047080), subfamily A2A non-peptidase homologues (MER047088), subfamily A2A non-peptidase homologues (MER047089), subfamily A2A non-peptidase homologues (MER047091), subfamily A2A non-peptidase homologues (MER047092), subfamily A2A non-peptidase homologues (MER047093), subfamily A2A non-peptidase homologues (MER047094), subfamily A2A non-peptidase homologues (MER047097), subfamily A2A non-peptidase homologues (MER047099), subfamily A2A non-peptidase homologues MER047101), subfamily A2A non-peptidase homologues (MER047102), subfamily A2A non-peptidase homologues (MER047107), subfamily A2A non-peptidase homologues (MER047108), subfamily A2A non-peptidase homologues (MER047109), subfamily A2A non-peptidase homologues (MER047110), subfamily A2A non-peptidase homologues MER047111), subfamily A2A non-peptidase homologues (MER047114), subfamily A2A non-peptidase homologues (MER047118), subfamily A2A non-peptidase homologues (MER047121), subfamily A2A non-peptidase homologues (MER047122), subfamily A2A non-peptidase homologues (MER047126), subfamily A2A non-peptidase homologues (MER047129), subfamily A2A non-peptidase homologues (MER047130), subfamily A2A non-peptidase homologues (MER047134), subfamily A2A non-peptidase homologues (MER047135), subfamily A2A non-peptidase homologues (MER047137), subfamily A2A non-peptidase homologues (MER047140), subfamily A2A non-peptidase homologues (MER047141), subfamily A2A non-peptidase homologues (MER047142), subfamily A2A non-peptidase homologues (MER047148), subfamily A2A non-peptidase homologues (MER047149), subfamily A2A non-peptidase homologues (MER047151), subfamily A2A non-peptidase homologues (MER047154), subfamily A2A non-peptidase homologues (MER047155), subfamily A2A non-peptidase homologues (MER047156), subfamily A2A non-peptidase homologues (MER047157), subfamily A2A non-peptidase homologues (MER047159), subfamily A2A non-peptidase homologues (MER047161), subfamily A2A non-peptidase homologues (MER047163), subfamily A2A non-peptidase homologues (MER047166), subfamily A2A non-peptidase homologues (MER047171), subfamily A2A non-peptidase homologues (MER047173), subfamily A2A non-peptidase homologues (MER047174), subfamily A2A non-peptidase homologues (MER047179), subfamily A2A non-peptidase homologues (MER047183), subfamily A2A non-peptidase homologues (MER047186), subfamily A2A non-peptidase homologues (MER047190), subfamily A2A non-peptidase homologues (MER047191), subfamily A2A non-peptidase homologues (MER047196), subfamily A2A non-peptidase homologues (MER047198), subfamily A2A non-peptidase homologues (MER047199), subfamily A2A non-peptidase homologues (MER047201), subfamily A2A non-peptidase homologues (MER047202), subfamily A2A non-peptidase homologues (MER047203), subfamily A2A non-peptidase homologues (MER047204), subfamily A2A non-peptidase homologues (MER047205), subfamily A2A non-peptidase homologues (MER047207), subfamily A2A non-peptidase homologues (MER047208), subfamily A2A non-peptidase homologues (MER047210), subfamily A2A non-peptidase homologues (MER047211), subfamily A2A non-peptidase homologues (MER047212), subfamily A2A non-peptidase homologues (MER047213), subfamily A2A non-peptidase homologues (MER047215), subfamily A2A non-peptidase homologues (MER047216), subfamily A2A non-peptidase homologues (MER047218), subfamily A2A non-peptidase homologues (MER047219), subfamily A2A non-peptidase homologues (MER047221), subfamily A2A non-peptidase homologues (MER047224), subfamily A2A non-peptidase homologues (MER047225), subfamily A2A non-peptidase homologues (MER047226), subfamily A2A non-peptidase homologues (MER047227), subfamily A2A non-peptidase homologues (MER047230), subfamily A2A non-peptidase homologues (MER047232), subfamily A2A non-peptidase homologues (MER047233), subfamily A2A non-peptidase homologues (MER047234), subfamily A2A non-peptidase homologues (MER047236), subfamily A2A non-peptidase homologues (MER047238), subfamily A2A non-peptidase homologues (MER047239), subfamily A2A non-peptidase homologues (MER047240), subfamily A2A non-peptidase homologues (MER047242), subfamily A2A non-peptidase homologues (MER047243), subfamily A2A non-peptidase homologues (MER047249), subfamily A2A non-peptidase homologues (MER047251), subfamily A2A non-peptidase homologues (MER047252), subfamily A2A non-peptidase homologues (MER047254), subfamily A2A non-peptidase homologues (MER047255), subfamily A2A non-peptidase homologues (MER047263), subfamily A2A non-peptidase homologues (MER047265), subfamily A2A non-peptidase homologues (MER047266), subfamily A2A non-peptidase homologues (MER047267), subfamily A2A non-peptidase homologues (MER047268), subfamily A2A non-peptidase homologues (MER047269), subfamily A2A non-peptidase homologues (MER047272), subfamily A2A non-peptidase homologues (MER047273), subfamily A2A non-peptidase homologues (MER047274), subfamily A2A non-peptidase homologues (MER047275), subfamily A2A non-peptidase homologues (MER047276), subfamily A2A non-peptidase homologues (MER047279), subfamily A2A non-peptidase homologues (MER047280), subfamily A2A non-peptidase homologues (MER047281), subfamily A2A non-peptidase homologues (MER047282), subfamily A2A non-peptidase homologues (MER047284), subfamily A2A non-peptidase homologues (MER047285), subfamily A2A non-peptidase homologues (MER047289), subfamily A2A non-peptidase homologues (MER047290), subfamily A2A non-peptidase homologues (MER047294), subfamily A2A non-peptidase homologues (MER047295), subfamily A2A non-peptidase homologues (MER047298), subfamily A2A non-peptidase homologues (MER047300), subfamily A2A non-peptidase homologues (MER047302), subfamily A2A non-peptidase homologues (MER047304), subfamily A2A non-peptidase homologues (MER047305), subfamily A2A non-peptidase homologues (MER047306), subfamily A2A non-peptidase homologues (MER047307), subfamily A2A non-peptidase homologues (MER047310), subfamily A2A non-peptidase homologues (MER047311), subfamily A2A non-peptidase homologues (MER047314), subfamily A2A non-peptidase homologues (MER047318), subfamily A2A non-peptidase homologues (MER047320), subfamily A2A non-peptidase homologues (MER047321), subfamily A2A non-peptidase homologues (MER047322), subfamily A2A non-peptidase homologues (MER047326), subfamily A2A non-peptidase homologues (MER047327), subfamily A2A non-peptidase homologues (MER047330), subfamily A2A non-peptidase homologues (MER047333), subfamily A2A non-peptidase homologues (MER047362), subfamily A2A non-peptidase homologues (MER047366), subfamily A2A non-peptidase homologues (MER047369), subfamily A2A non-peptidase homologues (MER047370), subfamily A2A non-peptidase homologues (MER047371), subfamily A2A non-peptidase homologues (MER047375), subfamily A2A non-peptidase homologues (MER047376), subfamily A2A non-peptidase homologues (MER047381), subfamily A2A non-peptidase homologues (MER047383), subfamily A2A non-peptidase homologues (MER047384), subfamily A2A non-peptidase homologues (MER047385), subfamily A2A non-peptidase homologues (MER047388), subfamily A2A non-peptidase homologues (MER047389), subfamily A2A non-peptidase homologues (MER047391), subfamily A2A non-peptidase homologues (MER047394), subfamily A2A non-peptidase homologues (MER047396), subfamily A2A non-peptidase homologues (MER047400), subfamily A2A non-peptidase homologues (MER047401), subfamily A2A non-peptidase homologues (MER047403), subfamily A2A non-peptidase homologues (MER047406), subfamily A2A non-peptidase homologues (MER047407), subfamily A2A non-peptidase homologues (MER047410), subfamily A2A non-peptidase homologues (MER047411), subfamily A2A non-peptidase homologues (MER047413), subfamily A2A non-peptidase homologues (MER047414), subfamily A2A non-peptidase homologues (MER047416), subfamily A2A non-peptidase homologues (MER047417), subfamily A2A non-peptidase homologues (MER047420), subfamily A2A non-peptidase homologues (MER047423), subfamily A2A non-peptidase homologues (MER047424), subfamily A2A non-peptidase homologues (MER047428), subfamily A2A non-peptidase homologues (MER047429), subfamily A2A non-peptidase homologues (MER047431), subfamily A2A non-peptidase homologues (MER047434), subfamily A2A non-peptidase homologues (MER047439), subfamily A2A non-peptidase homologues (MER047442), subfamily A2A non-peptidase homologues (MER047445), subfamily A2A non-peptidase homologues (MER047449), subfamily A2A non-peptidase homologues (MER047450), subfamily A2A non-peptidase homologues (MER047452), subfamily A2A non-peptidase homologues (MER047455), subfamily A2A non-peptidase homologues (MER047457), subfamily A2A non-peptidase homologues (MER047458), subfamily A2A non-peptidase homologues (MER047459), subfamily A2A non-peptidase homologues (MER047463), subfamily A2A non-peptidase homologues (MER047468), subfamily A2A non-peptidase homologues (MER047469), subfamily A2A non-peptidase homologues (MER047470), subfamily A2A non-peptidase homologues (MER047476), subfamily A2A non-peptidase homologues (MER047478), subfamily A2A non-peptidase homologues (MER047483), subfamily A2A non-peptidase homologues (MER047488), subfamily A2A non-peptidase homologues (MER047489), subfamily A2A non-peptidase homologues (MER047490), subfamily A2A non-peptidase homologues (MER047493), subfamily A2A non-peptidase homologues (MER047494), subfamily A2A non-peptidase homologues (MER047495), subfamily A2A non-peptidase homologues (MER047496), subfamily A2A non-peptidase homologues (MER047497), subfamily A2A non-peptidase homologues (MER047499), subfamily A2A non-peptidase homologues (MER047502), subfamily A2A non-peptidase homologues (MER047504), subfamily A2A non-peptidase homologues (MER047511), subfamily A2A non-peptidase homologues (MER047513), subfamily A2A non-peptidase homologues (MER047514), subfamily A2A non-peptidase homologues (MER047515), subfamily A2A non-peptidase homologues (MER047516), subfamily A2A non-peptidase homologues (MER047520), subfamily A2A non-peptidase homologues (MER047533), subfamily A2A non-peptidase homologues (MER047537), subfamily A2A non-peptidase homologues (MER047569), subfamily A2A non-peptidase homologues (MER047570), subfamily A2A non-peptidase homologues (MER047584), subfamily A2A non-peptidase homologues (MER047603), subfamily A2A non-peptidase homologues (MER047604), subfamily A2A non-peptidase homologues (MER047606), subfamily A2A non-peptidase homologues (MER047609), subfamily A2A non-peptidase homologues (MER047616), subfamily A2A non-peptidase homologues (MER047619), subfamily A2A non-peptidase homologues (MER047648), subfamily A2A non-peptidase homologues (MER047649), subfamily A2A non-peptidase homologues (MER047662), subfamily A2A non-peptidase homologues (MER048004), subfamily A2A non-peptidase homologues (MER048018), subfamily A2A non-peptidase homologues (MER048019), subfamily A2A non-peptidase homologues (MER048023), subfamily A2A non-peptidase homologues (MER048037), subfamily A2A unassigned peptidases (MER047164), subfamily A2A unassigned peptidases (MER047231), subfamily A2A unassigned peptidases (MER047386), skin aspartic protease (MER057097), presenilin 1 (MER005221), presenilin 2 (MER005223), impas 1 peptidase (MER019701), impas 1 peptidase (MER184722), impas 4 peptidase (MER019715), impas 2 peptidase (MER019708), impas 5 peptidase (MER019712), impas 3 peptidase (MER019711), possible family A22 pseudogene (Homo sapiens chromosome 18) (MER029974), possible family A22 pseudogene (Homo sapiens chromosome 11) (MER023159), cathepsin V (MER004437), cathepsin X (MER004508), cathepsin F (MER004980), cathepsin L (MER000622), cathepsin S (MER000633), cathepsin O (MER001690), cathepsin K (MER000644), cathepsin W (MER003756), cathepsin H (MER000629), cathepsin B (MER000686), dipeptidyl-peptidase I (MER001937), bleomycin hydrolase (animal) (MER002481), tubulointerstitial nephritis antigen (MER016137), tubulointerstitial nephritis antigen-related protein (MER021799), cathepsin L-like pseudogene 1 (Homo sapiens) (MER002789), cathepsin B-like pseudogene (chromosome 4, Homo sapiens) (MER029469), cathepsin B-like pseudogene (chromosome 1, Homo sapiens) (MER029457), CTSLL2 g.p. (Homo sapiens) (MER005210), CTSLL3 g.p. (Homo sapiens) (MER005209), calpain-1 (MER000770), calpain-2 (MER000964), calpain-3 (MER001446), calpain-9 (MER004042), calpain-8 (MER021474), calpain-15 (MER004745), calpain-5 (MER002939), calpain-11 (MER005844), calpain-12 (MER029889), calpain-10 (MER013510), calpain-13 (MER020139), calpain-14 (MER029744), Mername-AA253 peptidase (MER005537), calpamodulin (MER000718), hypothetical protein 940251 (MER003201), ubiquitinyl hydrolase-L1 (MER000832), ubiquitinyl hydrolase-L3 (MER000836), ubiquitinyl hydrolase-BAP1 (MER003989), ubiquitinyl hydrolase-UCH37 (MER005539), ubiquitin-specific peptidase 5 (MER002066), ubiquitin-specific peptidase 6 (MER000863), ubiquitin-specific peptidase 4 (MER001795), ubiquitin-specific peptidase 8 (MER001884), ubiquitin-specific peptidase 13 (MER002627), ubiquitin-specific peptidase 2 (MER004834), ubiquitin-specific peptidase 11 (MER002693), ubiquitin-specific peptidase 14 (MER002667), ubiquitin-specific peptidase 7 (MER002896), ubiquitin-specific peptidase 9X (MER005877), ubiquitin-specific peptidase 10 (MER004439), ubiquitin-specific peptidase 1 (MER004978), ubiquitin-specific peptidase 12 (MER005454), ubiquitin-specific peptidase 16 (MER005493), ubiquitin-specific peptidase 15 (MER005427), ubiquitin-specific peptidase 17 (MER002900), ubiquitin-specific peptidase 19 (MER005428), ubiquitin-specific peptidase 20 (MER005494), ubiquitin-specific peptidase 3 (MER005513), ubiquitin-specific peptidase 9Y (MER004314), ubiquitin-specific peptidase 18 (MER005641), ubiquitin-specific peptidase 21 (MER006258), ubiquitin-specific peptidase 22 (MER012130), ubiquitin-specific peptidase 33 (MER014335), ubiquitin-specific peptidase 29 (MER012093), ubiquitin-specific peptidase 25 (MER011115), ubiquitin-specific peptidase 36 (MER014033), ubiquitin-specific peptidase 32 (MER014290), ubiquitin-specific peptidase 26 (Homo sapiens-type) (MER014292), ubiquitin-specific peptidase 24 (MER005706), ubiquitin-specific peptidase 42 (MER011852), ubiquitin-specific peptidase 46 (MER014629), ubiquitin-specific peptidase 37 (MER014633), ubiquitin-specific peptidase 28 (MER014634), ubiquitin-specific peptidase 47 (MER014636), ubiquitin-specific peptidase 38 (MER014637), ubiquitin-specific peptidase 44 (MER014638), ubiquitin-specific peptidase 50 (MER030315), ubiquitin-specific peptidase 35 (MER014646), ubiquitin-specific peptidase 30 (MER014649), Mername-AA091 peptidase (MER014743), ubiquitin-specific peptidase 45 (MER030314), ubiquitin-specific peptidase 51 (MER014769), ubiquitin-specific peptidase 34 (MER014780), ubiquitin-specific peptidase 48 (MER064620), ubiquitin-specific peptidase 40 (MER015483), ubiquitin-specific peptidase 41 (MER045268), ubiquitin-specific peptidase 31 (MER015493), Mername-AA129 peptidase (MER016485), ubiquitin-specific peptidase 49 (MER016486), Mername-AA187 peptidase (MER052579), USP17-like peptidase (MER030192), ubiquitin-specific peptidase 54 (MER028714), ubiquitin-specific peptidase 53 (MER027329), ubiquitin-specific endopeptidase 39 [misleading] (MER064621), Mername-AA090 non-peptidase homologue (MER014739), ubiquitin-specific peptidase 43 [misleading] (MER030140), ubiquitin-specific peptidase 52 [misleading] (MER030317), NEK2 pseudogene (MER014736), C19 pseudogene (Homo sapiens: chromosome 5) (MER029972), Mername-AA088 peptidase (MER014750), autophagin-2 (MER013564), autophagin-1 (MER013561), autophagin-3 (MER014316), autophagin-4 (MER064622), Cezanne deubiquitinylating peptidase (MER029042), Cezanne-2 peptidase (MER029044), tumor necrosis factor alpha-induced protein 3 (MER029050), trabid peptidase (MER029052), VCIP135 deubiquitinating peptidase (MER152304), otubain-1 (MER029056), otubain-2 (MER029061), CylD protein (MER030104), UfSP1 peptidase (MER042724), UfSP2 peptidase (MER060306), DUBA deubiquitinylating enzyme (MER086098), KIAA0459 (Homo sapiens)-like protein (MER 122467), Otud1 protein (MER125457), glycosyltransferase 28 domain containing 1, isoform CRA_c (Homo sapiens)-like (MER123606), hin1L g.p. (Homo sapiens) (MER139816), ataxin-3 (MER099998), ATXN3L putative peptidase (MER115261), Josephin domain containing 1 (Homo sapiens) (MER125334), Josephin domain containing 2 (Homo sapiens) (MER 124068), YOD1 peptidase (MER116559), legumain (plant alpha form) (MER044591), legumain (MER001800), glycosylphosphatidylinositol: protein transamidase (MER002479), legumain pseudogene (Homo sapiens) (MER029741), family C13 unassigned peptidases (MER 175813), caspase-1 (MER000850), caspase-3 (MER000853), caspase-7 (MER002705), caspase-6 (MER002708), caspase-2 (MER001644), caspase-4 (MER001938), caspase-5 (MER002240), caspase-8 (MER002849), caspase-9 (MER002707), caspase-10 (MER002579), caspase-14 (MER012083), paracaspase (MER019325), Mername-AA143 peptidase (MER021304), Mername-AA186 peptidase (MER020516), putative caspase (Homo sapiens) (MER021463), FLIP protein (MER003026), Mername-AA142 protein (MER021316), caspase-12 pseudogene (Homo sapiens) (MER019698), Mername-AA093 caspase pseudogene (MER014766), subfamily C14A non-peptidase homologues (MER185329), subfamily C14A non-peptidase homologues (MER179956), separase (Homo sapiens-type) (MER011775), separase-like pseudogene (MER014797), SENP1 peptidase (MER011012), SENP3 peptidase (MER011019), SENP6 peptidase (MER011109), SENP2 peptidase (MER012183), SENP5 peptidase (MER014032), SENP7 peptidase (MER014095), SENP8 peptidase (MER016161), SENP4 peptidase (MER005557), pyroglutamyl-peptidase I (chordate) (MER011032), Mername-AA073 peptidase (MER029978), Sonic hedgehog protein (MER002539), Indian hedgehog protein (MER002538), Desert hedgehog protein (MER012170), dipeptidyl-peptidase III (MER004252), Mername-AA164 protein (MER020410), LOC138971 g.p. (Homo sapiens) (MER020074), Atp23 peptidase (MER060642), prenyl peptidase 1 (MER004246), aminopeptidase N (MER000997), aminopeptidase A (MER001012), leukotriene A4 hydrolase (MER001013), pyroglutamyl-peptidase II (MER012221), cytosol alanyl aminopeptidase (MER002746), cystinyl aminopeptidase (MER002060), aminopeptidase B (MER001494), aminopeptidase PILS (MER005331), arginyl aminopeptidase-like 1 (MER012271), leukocyte-derived arginine aminopeptidase (MER002968), aminopeptidase Q (MER052595), aminopeptidase O (MER019730), Tata binding protein associated factor (MER026493), angiotensin-converting enzyme peptidase unit 1 (MER004967), angiotensin-converting enzyme peptidase unit 2 (MER001019), angiotensin-converting enzyme-2 (MER011061), Mername-AA153 protein (MER020514), thimet oligopeptidase (MER001737), neurolysin (MER010991), mitochondrial intermediate peptidase (MER003665), Mername-AA154 protein (MER021317), leishmanolysin-2 (MER014492), leishmanolysin-3 (MER180031), matrix metallopeptidase-1 (MER001063), matrix metallopeptidase-8 (MER001084), matrix metallopeptidase-2 (MER001080), matrix metallopeptidase-9 (MER001085), matrix metallopeptidase-3 (MER001068), matrix metallopeptidase-10 (Homo sapiens-type) (MER001072), matrix metallopeptidase-11 (MER001075), matrix metallopeptidase-7 (MER001092), matrix metallopeptidase-12 (MER001089), matrix metallopeptidase-13 (MER001411), membrane-type matrix metallopeptidase-1 (MER001077), membrane-type matrix metallopeptidase-2 (MER002383), membrane-type matrix metallopeptidase-3 (MER002384), membrane-type matrix metallopeptidase-4 (MER002595), matrix metallopeptidase-20 (MER003021), matrix metallopeptidase-19 (MER002076), matrix metallopeptidase-23B (MER004766), membrane-type matrix metallopeptidase-5 (MER005638), membrane-type matrix metallopeptidase-6 (MER012071), matrix metallopeptidase-21 (MER006101), matrix metallopeptidase-22 (MER014098), matrix metallopeptidase-26 (MER012072), matrix metallopeptidase-28 (MER013587), matrix metallopeptidase-23A (MER037217), macrophage elastase homologue (chromosome 8, Homo sapiens) (MER030035), Mername-AA156 protein (MER021309), matrix metallopeptidase-like 1 (MER045280), subfamily M10A non-peptidase homologues (MER175912), subfamily M10A non-peptidase homologues (MER187997), subfamily M10A non-peptidase homologues (MER187998), subfamily M10A non-peptidase homologues (MER180000), meprin alpha subunit (MER001111), meprin beta subunit (MER005213), procollagen C-peptidase (MER001113), mammalian tolloid-like 1 protein (MER005124), mammalian-type tolloid-like 2 protein (MER005866), ADAMTS9 peptidase (MER012092), ADAMTS14 peptidase (MER016700), ADAMTS15 peptidase (MER017029), ADAMTS16 peptidase (MER015689), ADAMTS17 peptidase (MER016302), ADAMTS18 peptidase (MER016090), ADAMTS19 peptidase (MER015663), ADAM8 peptidase (MER003902), ADAM9 peptidase (MER001140), ADAM10 peptidase (MER002382), ADAM12 peptidase (MER005107), ADAM19 peptidase (MER012241), ADAM15 peptidase (MER002386), ADAM17 peptidase (MER003094), ADAM20 peptidase (MER004725), ADAMDEC1 peptidase (MER000743), ADAMTS3 peptidase (MER005100), ADAMTS4 peptidase (MER005101), ADAMTS1 peptidase (MER005546), ADAM28 peptidase (Homo sapiens-type) (MER005495), ADAMTS5 peptidase (MER005548), ADAMTS8 peptidase (MER005545), ADAMTS6 peptidase (MER005893), ADAMTS7 peptidase (MER005894), ADAM30 peptidase (MER006268), ADAM21 peptidase (Homo sapiens-type) (MER004726), ADAMTS10 peptidase (MER014331), ADAMTS12 peptidase (MER014337), ADAMTS13 peptidase (MER015450), ADAM33 peptidase (MER015143), ovastacin (MER029996), ADAMTS20 peptidase (Homo sapiens-type) (MER026906), procollagen I N-peptidase (MER004985), ADAM2 protein (MER003090), ADAM6 protein (MER047044), ADAM7 protein (MER005109), ADAM18 protein (MER012230), ADAM32 protein (MER026938), non-peptidase homologue (Homo sapiens chromosome 4) (MER029973), family M12 non-peptidase homologue (Homo sapiens chromosome 16) (MER047654), family M12 non-peptidase homologue (Homo sapiens chromosome 15) (MER047250), ADAM3B protein (Homo sapiens-type) (MER005199), ADAM11 protein (MER001146), ADAM22 protein (MER005102), ADAM23 protein (MER005103), ADAM29 protein (MER006267), protein similar to ADAM21 peptidase preproprotein (Homo sapiens) (MER026944), Mername-AA225 peptidase homologue (Homo sapiens) (MER047474), putative ADAM pseudogene (chromosome 4, Homo sapiens) (MER029975), ADAM3A g.p. (Homo sapiens) (MER005200), ADAM1 g.p. (Homo sapiens) (MER003912), subfamily M12B non-peptidase homologues (MER188210), subfamily M12B non-peptidase homologues (MER188211), subfamily M12B non-peptidase homologues (MER188212), subfamily M12B non-peptidase homologues (MER188220), neprilysin (MER001050), endothelin-converting enzyme 1 (MER001057), endothelin-converting enzyme 2 (MER004776), DINE peptidase (MER005197), neprilysin-2 (MER013406), Kell blood-group protein (MER001054), PHEX peptidase (MER002062), i-AAA peptidase (MER001246), i-AAA peptidase (MER005755), paraplegin (MER004454), Afg3-like protein 2 (MER005496), Afg3-like protein 1A (MER014306), pappalysin-1 (MER002217), pappalysin-2 (MER014521), farnesylated-protein converting enzyme 1 (MER002646), metalloprotease-related protein-1 (MER030873), aminopeptidase AMZ2 (MER011907), aminopeptidase AMZ1 (MER058242), carboxypeptidase A1 (MER001190), carboxypeptidase A2 (MER001608), carboxypeptidase B (MER001194), carboxypeptidase N (MER001198), carboxypeptidase E (MER001199), carboxypeptidase M (MER001205), carboxypeptidase U (MER001193), carboxypeptidase A3 (MER001187), metallocarboxypeptidase D peptidase unit 1 (MER003781), metallocarboxypeptidase Z (MER003428), metallocarboxypeptidase D peptidase unit 2 (MER004963), carboxypeptidase A4 (MER013421), carboxypeptidase A6 (MER013456), carboxypeptidase A5 (MER017121), metallocarboxypeptidase O (MER016044), cytosolic carboxypeptidase-like protein 5 (MER033174), cytosolic carboxypeptidase 3 (MER033176), cytosolic carboxypeptidase 6 (MER033178), cytosolic carboxypeptidase 1 (MER033179), cytosolic carboxypeptidase 2 (MER037713), metallocarboxypeptidase D non-peptidase unit (MER004964), adipocyte-enhancer binding protein 1 (MER003889), carboxypeptidase-like protein X1 (MER013404), carboxypeptidase-like protein X2 (MER078764), cytosolic carboxypeptidase (MER026952), family M14 non-peptidase homologues (MER199530), insulysin (MER001214), mitochondrial processing peptidase beta-subunit (MER004497), nardilysin (MER003883), cupitrilysin (MER004877), mitochondrial processing peptidase non-peptidase alpha subunit (MER001413), ubiquinol-cytochrome c reductase core protein I (MER003543), ubiquinol-cytochrome c reductase core protein II (MER003544), ubiquinol-cytochrome c reductase core protein domain 2 (MER043998), insulysin unit 2 (MER046821), nardilysin unit 2 (MER046874), insulysin unit 3 (MER078753), mitochondrial processing peptidase subunit alpha unit 2 (MER124489), nardilysin unit 3 (MER142856), LOC133083 g.p. (Homo sapiens) (MER021876), subfamily M16B non-peptidase homologues (MER188757), leucyl aminopeptidase (animal) (MER003100), Mername-AA040 peptidase (MER003919), leucyl aminopeptidase-1 (Caenorhabditis-type) (MER013416), methionyl aminopeptidase 1 (MER001342), methionyl aminopeptidase 2 (MER001728), aminopeptidase P2 (MER004498), Xaa-Pro dipeptidase (eukaryote) (MER001248), aminopeptidase PI (MER004321), mitochondrial intermediate cleaving peptidase 55 kDa (MER013463), mitochondrial methionyl aminopeptidase (MER014055), Mername-AA020 peptidase homologue (MER010972), proliferation-association protein 1 (MER005497), chromatin-specific transcription elongation factor 140 kDa subunit (MER026495), proliferation-associated protein 1-like (Homo sapiens chromosome X) (MER029983), Mername-AA226 peptidase homologue (Homo sapiens) (MER056262), Mername-AA227 peptidase homologue (Homo sapiens) (MER047299), subfamily M24A non-peptidase homologues (MER179893), aspartyl aminopeptidase (MER003373), Gly-Xaa carboxypeptidase (MER033182), carnosine dipeptidase II (MER014551), carnosine dipeptidase I (MER015142), Mername-AA161 protein (MER021873), aminoacylase (MER001271), glutamate carboxypeptidase II (MER002104), NAALADASE L peptidase (MER005239), glutamate carboxypeptidase III (MER005238), plasma glutamate carboxypeptidase (MER005244), Mername-AA103 peptidase (MER015091), Fxna peptidase (MER029965), transferrin receptor protein (MER002105), transferrin receptor 2 protein (MER005152), glutaminyl cyclise (MER015095), glutamate carboxypeptidase II (Homo sapiens)-type non-peptidase homologue (MER026971), nicalin (MER044627), membrane dipeptidase (MER001260), membrane-bound dipeptidase-2 (MER013499), membrane-bound dipeptidase-3 (MER013496), dihydro-orotase (MER005767), dihydropyrimidinase (MER033266), dihydropyrimidinase related protein-1 (MER030143), dihydropyrimidinase related protein-2 (MER030155), dihydropyrimidinase related protein-3 (MER030151), dihydropyrimidinase related protein-4 (MER030149), dihydropyrimidinase related protein-5 (MER030136), hypothetical protein like 5730457F11RIK (MER033184), 1300019j08rik protein (MER033186)), guanine aminohydrolase (MER037714), Kael putative peptidase (MER001577), OSGEPL1-like protein (MER013498), S2P peptidase (MER004458), subfamily M23B non-peptidase homologues (MER 199845), subfamily M23B non-peptidase homologues (MER 199846), subfamily M23B non-peptidase homologues (MER199847), subfamily M23B non-peptidase homologues (MER137320), subfamily M23B non-peptidase homologues (MER201557), subfamily M23B non-peptidase homologues (MER 199417), subfamily M23B non-peptidase homologues (MER199418), subfamily M23B non-peptidase homologues (MER199419), subfamily M23B non-peptidase homologues (MER 199420), subfamily M23B non-peptidase homologues (MER175932), subfamily M23B non-peptidase homologues (MER199665), Pohl peptidase (MER020382), Jab1/MPN domain metalloenzyme (MER022057), Mername-AA165 peptidase (MER021865), Brcc36 isopeptidase (MER021890), histone H2A deubiquitinase MYSM1 (MER021887), AMSH deubiquitinating peptidase (MER030146), putative peptidase (Homo sapiens chromosome 2) (MER029970), Mername-AA168 protein (MER021886), COP9 signalosome subunit 6 (MER030137), 26S proteasome non-ATPase regulatory subunit 7 (MER030134), eukaryotic translation initiation factor 3 subunit 5 (MER030133), IFP38 peptidase homologue (MER030132), subfamily M67A non-peptidase homologues (MER191181), subfamily M67A unassigned peptidases (MER191144), granzyme B (Homo sapiens-type) (MER000168), testisin (MER005212), tryptase beta (MER000136), kallikrein-related peptidase 5 (MER005544), corin (MER005881), kallikrein-related peptidase 12 (MER006038), DESC1 peptidase (MER006298), tryptase gamma 1 (MER011036), kallikrein-related peptidase 14 (MER011038), hyaluronan-binding peptidase (MER003612), transmembrane peptidase, serine 4 (MER011104), intestinal serine peptidase (rodent) (MER016130), adrenal secretory serine peptidase (MER003734), tryptase delta 1 (Homo sapiens) (MER005948), matriptase-3 (MER029902), marapsin (MER006119), tryptase-6 (MER006118), ovochymase-1 domain 1 (MER099182), transmembrane peptidase, serine 3 (MER005926), kallikrein-related peptidase 15 (MER000064), Mername-AA031 peptidase (MER014054), TMPRSS13 peptidase (MER014226), Mername-AA038 peptidase (MER062848), Mername-AA204 peptidase (MER029980), cationic trypsin (Homo sapiens-type) (MER000020), elastase-2 (MER000118), mannan-binding lectin-associated serine peptidase-3 (MER031968), cathepsin G (MER000082), myeloblastin (MER000170), granzyme A (MER001379), granzyme M (MER001541), chymase (Homo sapiens-type) (MER000123), tryptase alpha (MER000135), granzyme K (MER001936), granzyme H (MER000166), chymotrypsin B (MER000001), elastase-1 (MER003733), pancreatic endopeptidase E (MER000149), pancreatic elastase II (MER000146), enteropeptidase (MER002068), chymotrypsin C (MER000761), prostasin (MER002460), kallikrein 1 (MER000093), kallikrein-related peptidase 2 (MER000094), kallikrein-related peptidase 3 (MER000115), mesotrypsin (MER000022), complement component C1r-like peptidase (MER016352), complement factor D (MER000130), complement component activated C1r (MER000238), complement component activated C1s (MER000239), complement component C2a (MER000231), complement factor B (MER000229), mannan-binding lectin-associated serine peptidase 1 (MER000244), complement factor I (MER000228), pancreatic endopeptidase E form B (MER000150), pancreatic elastase IIB (MER000147), coagulation factor XIIa (MER000187), plasma kallikrein (MER000203) coagulation factor Xia (MER000210), coagulation factor IXa (MER000216), coagulation factor Vila (MER000215), coagulation factor Xa (MER000212), thrombin (MER000188), protein C (activated) (MER000222), acrosin (MER000078), hepsin (MER000156), hepatocyte growth factor activator (MER000186), mannan-binding lectin-associated serine peptidase 2 (MER002758), u-plasminogen activator (MER000195), t-plasminogen activator (MER000192), plasmin (MER000175), kallikrein-related peptidase 6 (MER002580), neurotrypsin (MER004171), kallikrein-related peptidase 8 (MER005400), kallikrein-related peptidase 10 (MER003645), epitheliasin (MER003736), kallikrein-related peptidase 4 (MER005266), prosemin (MER004214), chymopasin (MER001503), kallikrein-related peptidase 11 (MER004861), kallikrein-related peptidase 11 (MER216142), trypsin-2 type A (MER000021), HtrA1 peptidase (Homo sapiens-type) (MER002577), HtrA2 peptidase (MER208413), HtrA2 peptidase (MER004093), HtrA3 peptidase (MER014795), HtrA4 peptidase (MER016351), Tysnd1 peptidase (MER050461), TMPRSS12 peptidase (MER017085), HAT-like putative peptidase 2 (MER021884), trypsin C (MER021898), kallikrein-related peptidase 7 (MER002001), matriptase (MER003735), kallikrein-related peptidase 13 (MER005269), kallikrein-related peptidase 9 (MER005270), matriptase-2 (MER005278), umbilical vein peptidase (MER005421), LCLP peptidase (MER001900), spinesin (MER014385), marapsin-2 (MER021929), complement factor D-like putative peptidase (MER056164), ovochymase-2 (MER022410), HAT-like 4 peptidase (MER044589), ovochymase 1 domain 1 (MER022412), epidermis-specific SP-like putative peptidase (MER029900), testis serine peptidase 5 (MER029901), Mername-AA258 peptidase (MER000285), polyserase-IA unit 1 (MER030879), polyserase-IA unit 2 (MER030880), testis serine peptidase 2 (human-type) (MER033187), hypothetical acrosin-like peptidase (Homo sapiens) (MER033253), HAT-like 5 peptidase (MER028215), polyserase-3 unit 1 (MER061763), polyserase-3 unit 2 (MER061748), peptidase similar to tryptophan/serine protease (MER056263), polyserase-2 unit 1 (MER061777), Mername-AA123 peptidase (MER021930), HAT-like 2 peptidase (MER099184), hCG2041452-like protein (MER099172), hCG22067 (Homo sapiens) (MER099169), brain-rescue-factor-1 (Homo sapiens) (MER098873), hCG2041108 (Homo sapiens) (MER099173), polyserase-2 unit 2 (MER061760), polyserase-2 unit 3 (MER065694), Mername-AA201 (peptidase homologue) MER099175, secreted trypsin-like serine peptidase homologue (MER030000), polyserase-1A unit 3 (MER029880), azurocidin (MER000119), haptoglobin-1 (MER000233), haptoglobin-related protein (MER000235), macrophage-stimulating protein (MER001546), hepatocyte growth factor (MER000185), protein Z (MER000227), TESP1 protein (MER047214), LOC136242 protein (MER016132), plasma kallikrein-like protein 4 (MER016346), PRSS35 protein (MER016350), DKFZp586H2123-like protein (MER066474), apolipoprotein (MER000183), psi-KLK1 pseudogene (Homo sapiens) (MER033287), tryptase pseudogene I (MER015077), tryptase pseudogene II (MER015078), tryptase pseudogene III (MER015079), subfamily S1A unassigned peptidases (MER216982), subfamily S1A unassigned peptidases (MER216148), amidophosphoribosyltransferase precursor (MER003314), glutamine-fructose-6-phosphate transaminase 1 (MER003322), glutamine: fructose-6-phosphate amidotransferase (MER012158), Mername-AA144 protein (MER021319), asparagine synthetase (MER033254), family C44 non-peptidase homologues (MER159286), family C44 unassigned peptidases (MER185625) family C44 unassigned peptidases (MER185626), secernin 1 (MER045376), secernin 2 (MER064573), secernin 3 (MER064582), acid ceramidase precursor (MER100794), N-acylethanolamine acid amidase precursor (MER 141667), proteasome catalytic subunit 1 (MER000556), proteasome catalytic subunit 2 (MER002625), proteasome catalytic subunit 3 (MER002149), proteasome catalytic subunit 1i (MER000552), proteasome catalytic subunit 2i (MER001515), proteasome catalytic subunit 3i (MER000555), proteasome catalytic subunit 5t (MER026203), protein serine kinase c17 (MER026497), proteasome subunit alpha 6 (MER000557), proteasome subunit alpha 2 (MER000550), proteasome subunit alpha 4 (MER000554), proteasome subunit alpha 7 (MER033250), proteasome subunit alpha 5 (MER000558), proteasome subunit alpha 1 (MER000549), proteasome subunit alpha 3 (MER000553), proteasome subunit XAPC7 (MER004372), proteasome subunit beta 3 (MER001710), proteasome subunit beta 2 (MER002676), proteasome subunit beta 1 (MER000551), proteasome subunit beta 4 (MER001711), Mername-AA230 peptidase homologue (Homo sapiens) (MER047329), Mername-AA231 pseudogene (Homo sapiens) (MER047172), Mername-AA232 pseudogene (Homo sapiens) (MER047316), glycosylasparaginase precursor (MER003299), isoaspartyl dipeptidase (threonine type) (MER031622), taspase-1 (MER016969), gamma-glutamyltransferase 5 (mammalian-type) (MER001977), gamma-glutamyltransferase 1 (mammalian-type) (MER001629), gamma-glutamyltransferase 2 (Homo sapiens) (MER001976), gamma-glutamyltransferase-like protein 4 (MER002721), gamma-glutamyltransferase-like protein 3 (MER016970), similar to gamma-glutamyltransferase 1 precursor (Homo sapiens) (MER026204), similar to gamma-glutamyltransferase 1 precursor (Homo sapiens) (MER026205), Mername-AA211 putative peptidase (MER026207), gamma-glutamyltransferase 6 (MER159283), gamma-glutamyl transpeptidase homologue (chromosome 2, Homo sapiens) (MER037241), polycystin-1 (MER126824), KIAA1879 protein (MER 159329), polycystic kidney disease 1-like 3 (MER172554), gamma-glutamyl hydrolase (MER002963), guanine 5″-monophosphate synthetase (MER043387), carbamoyl-phosphate synthase (Homo sapiens-type) (MER078640), dihydro-orotase (N-terminal unit) (Homo sapiens-type) (MER060647), DJ-1 putative peptidase (MER003390), Mername-AA 100 putative peptidase (MER014802), Mername-AA101 non-peptidase homologue (MER014803), KIAA0361 protein (Homo sapiens-type) (MER042827), F1134283 protein (Homo sapiens) (MER044553), non-peptidase homologue chromosome 21 open reading frame 33 (Homo sapiens) (MER160094), family C56 non-peptidase homologues (MER177016), family C56 non-peptidase homologues (MER176613), family C56 non-peptidase homologues (MER176918), EGF-like module containing mucin-like hormone receptor-like 2 (MER037230), CD97 antigen (human type) (MER037286), EGF-like module containing mucin-like hormone receptor-like 3 (MER037288), EGF-like module containing mucin-like hormone receptor-like 1 (MER037278), EGF-like module containing mucin-like hormone receptor-like 4 (MER037294), cadherin EGF LAG seven-pass G-type receptor 2 precursor (Homo sapiens) (MER045397), Gpr64 (Mus musculus)-type protein (MER123205), GPR56 (Homo sapiens)-type protein (MER122057), latrophilin 2 (MER 122199), latrophilin-1 (MER126380), latrophilin 3 (MER 124612), protocadherin Flamingo 2 (MER 124239), ETL protein (MER 126267), G protein-coupled receptor 112 (MER126114), seven transmembrane helix receptor (MER 125448), Gpr114 protein (MER 159320), GPR126 vascular inducible G protein-coupled receptor (MER140015), GPR125 (Homo sapiens)-type protein (MER159279), GPR116 (Homo sapiens)-type G-protein coupled receptor (MER159280), GPR 128 (Homo sapiens)-type G-protein coupled receptor (MER 162015), GPR 133 (Homo sapiens)-type protein (MER159334), GPR 110 G-protein coupled receptor (MER 159277), GPR97 protein (MER159322), KPG_006 protein (MER 161773), KPG_008 protein (MER161835), KPG_009 protein (MER 159335), unassigned homologue (MER166269), GPR113 protein (MER159352), brain-specific angiogenesis inhibitor 2 (MER159746), PIDD auto-processing protein unit 1 (MER020001), PIDD auto-processing protein unit 2 (MER063690), MUC1 self-cleaving mucin (MER074260), dystroglycan (MER054741), proprotein convertase 9 (MER022416), site-1 peptidase (MER001948), furin (MER000375), proprotein convertase 1 (MER000376), proprotein convertase 2 (MER000377), proprotein convertase 4 (MER028255), PACE4 proprotein convertase (MER000383), proprotein convertase 5 (MER002578), proprotein convertase 7 (MER002984), tripeptidyl-peptidase II (MER000355), subfamily S8A non-peptidase homologues (MER201339), subfamily S8A non-peptidase homologues (MER191613), subfamily S8A unassigned peptidases (MER 191611), subfamily S8A unassigned peptidases (MER191612), subfamily S8A unassigned peptidases (MER191614), tripeptidyl-peptidase I (MER003575), prolyl oligopeptidase (MER000393), dipeptidyl-peptidase IV (eukaryote) (MER000401), acylaminoacyl-peptidase (MER000408), fibroblast activation protein alpha subunit (MER000399), PREPL A protein (MER004227), dipeptidyl-peptidase 8 (MER013484), dipeptidyl-peptidase 9 (MER004923), FLJ1 putative peptidase (MER017240), Mername-AA194 putative peptidase (MER017353), Mername-AA195 putative peptidase (MER017367), Mername-AA196 putative peptidase (MER017368), Mername-AA197 putative peptidase (MER017371), C14orf29 protein (MER033244), hypothetical protein (MER033245), hypothetical esterase/lipase/thioesterase (MER047309), protein bat5 (MER037840), hypothetical protein flj40219 (MER033212), hypothetical protein flj37464 (MER033240), hypothetical protein flj33678 (MER033241), dipeptidylpeptidase homologue DPP6 (MER000403), dipeptidylpeptidase homologue DPP10 (MER005988), protein similar to Mus musculus chromosome 20 open reading frame 135 (MER037845), kynurenine formamidase (MER046020), thyroglobulin precursor (MER011604), acetylcholinesterase (MER033188), cholinesterase (MER033198), carboxylesterase DI (MER033213), liver carboxylesterase (MER033220), carboxylesterase 3 (MER033224), carboxylesterase 2 (MER033226), bile salt-dependent lipase (MER033227), carboxylesterase-related protein (MER033231), neuroligin 3 (MER033232), neuroligin 4, X-linked (MER033235), neuroligin 4, Y-linked (MER033236), esterase D (MER043126), arylacetamide deacetylase (MER033237), KIAA1363-like protein (MER033242), hormone-sensitive lipase (MER033274), neuroligin 1 (MER033280), neuroligin 2 (MER033283), family S9 non-peptidase homologues (MER212939), family S9 non-peptidase homologues (MER211490), subfamily S9C unassigned peptidases (MER 192341), family S9 unassigned peptidases (MER209181), family S9 unassigned peptidases (MER200434), family S9 unassigned peptidases (MER209507), family S9 unassigned peptidases (MER209142), serine carboxypeptidase A (MER000430), vitellogenic carboxypeptidase-like protein (MER005492), RISC peptidase (MER010960), family S15 unassigned peptidases (MER 199442), family S15 unassigned peptidases (MER200437), family S15 unassigned peptidases (MER212825), lysosomal Pro-Xaa carboxypeptidase (MER000446), dipeptidyl-peptidase II (MER004952), thymus-specific serine peptidase (MER005538), epoxide hydrolase-like putative peptidase (MER031614), Loc328574-like protein (MER033246), abhydrolase domain-containing protein 4 (MER031616), epoxide hydrolase (MER000432), mesoderm specific transcript protein (MER199890), mesoderm specific transcript protein (MER017123), cytosolic epoxide hydrolase (MER029997), cytosolic epoxide hydrolase (MER213866), similar to hypothetical protein FLJ22408 (MER031608), CGI-58 putative peptidase (MER030163), Williams-Beuren syndrome critical region protein 21 epoxide hydrolase (MER031610), epoxide hydrolase (MER031612), hypothetical protein 922408 (epoxide hydrolase) (MER031617), monoglyceride lipase (MER033247), hypothetical protein (MER033249), valacyclovir hydrolase (MER033259), Ccg1-interacting factor b (MER210738), glycosylasparaginase precursor (MER003299), isoaspartyl dipeptidase (threonine type) (MER031622), taspase-1 (MER016969), gamma-glutamyltransferase 5 (mammalian-type) (MER001977), gamma-glutamyltransferase 1 (mammalian-type) (MER001629), gamma-glutamyltransferase 2 (Homo sapiens) (MER001976), gamma-glutamyltransferase-like protein 4 (MER002721), gamma-glutamyltransferase-like protein 3 (MER016970), similar to gamma-glutamyltransferase 1 precursor (Homo sapiens) (MER026204), similar to gamma-glutamyltransferase 1 precursor (Homo sapiens) (MER026205). Mername-AA211 putative peptidase (MER026207), gamma-glutamyltransferase 6 (MER 159283), gamma-glutamyl transpeptidase homologue (chromosome 2, Homo sapiens) (MER037241), polycystin-1 (MER126824), KIAA1879 protein (MER159329), polycystic kidney disease 1-like 3 (MER 172554), gamma-glutamyl hydrolase (MER002963), guanine 5″-monophosphate synthetase (MER043387), carbamoyl-phosphate synthase (Homo sapiens-type) (MER078640), dihydro-orotase (N-terminal unit) (Homo sapiens-type) (MER060647). DJ-1 putative peptidase (MER003390). Mername-AA100 putative peptidase (MER014802). Mername-AA101 non-peptidase homologue (MER014803). KIAA0361 protein (Homo sapiens-type) (MER042827). F1134283 protein (Homo sapiens) (MER044553), non-peptidase homologue chromosome 21 open reading frame 33 (Homo sapiens) (MER160094), family C56 non-peptidase homologues (MER 177016), family C56 non-peptidase homologues (MER176613), family C56 non-peptidase homologues (MER176918). EGF-like module containing mucin-like hormone receptor-like 2 (MER037230). CD97 antigen (human type) (MER037286). EGF-like module containing mucin-like hormone receptor-like 3 (MER037288). EGF-like module containing mucin-like hormone receptor-like 1 (MER037278). EGF-like module containing mucin-like hormone receptor-like 4 (MER037294). cadherin EGF LAG seven-pass G-type receptor 2 precursor (Homo sapiens) (MER045397), Gpr64 (Mus musculus)-type protein (MER 123205). GPR56 (Homo sapiens)-type protein (MER122057). latrophilin 2 (MER122199). latrophilin-1 (MER126380). latrophilin 3 (MER124612). protocadherin Flamingo 2 (MER124239). ETL protein (MER126267). G protein-coupled receptor 112 (MER126114). seven transmembrane helix receptor (MER125448). Gpr114 protein (MER159320). GPR 126 vascular inducible G protein-coupled receptor (MER140015). GPR125 (Homo sapiens)-type protein (MER159279). GPR116 (Homo sapiens)-type G-protein coupled receptor (MER159280). GPR128 (Homo sapiens)-type G-protein coupled receptor (MER162015). GPR 133 (Homo sapiens)-type protein (MER159334) GPR 110 G-protein coupled receptor (MER159277), GPR97 protein (MER159322), KPG_006 protein (MER161773) KPG_008 protein (MER161835), KPG_009 protein (MER159335), unassigned homologue (MER166269), GPR113 protein (MER159352), brain-specific angiogenesis inhibitor 2 (MER 159746), PIDD auto-processing protein unit 1 (MER020001), PIDD auto-processing protein unit 2 (MER063690), MUC1 self-cleaving mucin (MER074260), dystroglycan (MER054741), proprotein convertase 9 (MER022416), site-1 peptidase (MER001948), furin (MER000375), proprotein convertase 1 (MER000376), proprotein convertase 2 (MER000377), proprotein convertase 4 (MER028255), PACE4 proprotein convertase (MER000383), proprotein convertase 5 (MER002578), proprotein convertase 7 (MER002984), tripeptidyl-peptidase II (MER000355), subfamily S8A non-peptidase homologues (MER201339), subfamily S8A non-peptidase homologues (MER191613), subfamily S8A unassigned peptidases (MER191611), subfamily S8A unassigned peptidases (MER191612), subfamily S8A unassigned peptidases (MER191614), tripeptidyl-peptidase I (MER003575), prolyl oligopeptidase (MER000393), dipeptidyl-peptidase IV (eukaryote) (MER000401), acylaminoacyl-peptidase (MER000408), fibroblast activation protein alpha subunit (MER000399), PREPL A protein (MER004227), dipeptidyl-peptidase 8 (MER013484), dipeptidyl-peptidase 9 (MER004923), FLJ1 putative peptidase (MER017240), Mername-AA194 putative peptidase (MER017353), Mername-AA195 putative peptidase (MER017367), Mername-AA196 putative peptidase (MER017368), Mername-AA197 putative peptidase (MER017371), C14orf29 protein (MER033244), hypothetical protein (MER033245), hypothetical esterase/lipase/thioesterase (MER047309), protein bat5 (MER037840), hypothetical protein flj40219 (MER033212), hypothetical protein flj37464 (MER033240), hypothetical protein flj33678 (MER033241), dipeptidylpeptidase homologue DPP6 (MER000403), dipeptidylpeptidase homologue DPP10 (MER005988), protein similar to Mus musculus chromosome 20 open reading frame 135 (MER037845), kynurenine formamidase (MER046020), thyroglobulin precursor (MER011604), acetylcholinesterase (MER033188), cholinesterase (MER033198), carboxylesterase DI (MER033213), liver carboxylesterase (MER033220), carboxylesterase 3 (MER033224), carboxylesterase 2 (MER033226), bile salt-dependent lipase (MER033227), carboxylesterase-related protein (MER033231), neuroligin 3 (MER033232), neuroligin 4, X-linked (MER033235), neuroligin 4, Y-linked (MER033236), esterase D (MER043126), arylacetamide deacetylase (MER033237), KIAA1363-like protein (MER033242), hormone-sensitive lipase (MER033274), neuroligin 1 (MER033280), neuroligin 2 (MER033283), family S9 non-peptidase homologues (MER212939), family S9 non-peptidase homologues (MER211490), subfamily S9C unassigned peptidases (MER192341), family S9 unassigned peptidases (MER209181), family S9 unassigned peptidases (MER200434), family S9 unassigned peptidases (MER209507), family S9 unassigned peptidases (MER209142), serine carboxypeptidase A (MER000430), vitellogenic carboxypeptidase-like protein (MER005492), RISC peptidase (MER010960), family S15 unassigned peptidases (MER199442), family S15 unassigned peptidases (MER200437), family S15 unassigned peptidases (MER212825), lysosomal Pro-Xaa carboxypeptidase (MER000446), dipeptidyl-peptidase II (MER004952), thymus-specific serine peptidase (MER005538), epoxide hydrolase-like putative peptidase (MER031614), Loc328574-like protein (MER033246), abhydrolase domain-containing protein 4 (MER031616), epoxide hydrolase (MER000432), mesoderm specific transcript protein (MER 199890), mesoderm specific transcript protein (MER017123), cytosolic epoxide hydrolase (MER029997), cytosolic epoxide hydrolase (MER213866), similar to hypothetical protein FLJ22408 (MER031608), CGI-58 putative peptidase (MER030163), Williams-Beuren syndrome critical region protein 21 epoxide hydrolase (MER031610), epoxide hydrolase (MER031612), hypothetical protein flj22408 (epoxide hydrolase) (MER031617), monoglyceride lipase (MER033247), hypothetical protein (MER033249), valacyclovir hydrolase (MER033259), Ccg1-interacting factor b (MER210738).

Protease enzymatic activity can be regulated. For example, certain proteases can be inactivated by the presence or absence of a specific agent (e.g., that binds to the protease, such as specific small molecule inhibitors). Such proteases can be referred to as a “repressible protease.” Exemplary inhibitors for certain proteases are listed in Table 4B. For example, an NS3 protease can be repressed by a protease inhibitor including, but not limited to, simeprevir, danoprevir, asunaprevir, ciluprevir, boceprevir, sovaprevir, paritaprevir, telaprevir, grazoprevir, glecaprevir, and voxiloprevir. In another example, protease activity can be regulated through regulating expression of the protease itself, such as engineering a cell to express a protease using an inducible promoter system (e.g., Tet On/Off systems) or cell-specific promoters (promoters that can be used to express a heterologous protease are described in more detail in the Section herein titled “Promoters”). A protease can also contain a degron, such as any of the degrons described herein, and can be regulated using any of the degron systems described herein.

Protease enzymatic activity can also be regulated through selection of a specific protease cleavage site. For example, a protease cleavage site can be selected and/or engineered such that the sequence demonstrates a desired rate-of-cleavage by a desired protease, such as reduced cleavage kinetics relative to an endogenous sequence of a substrate naturally cleaved by the desired protease. As another example, a protease cleavage site can be selected and/or engineered such that the sequence demonstrates a desired rate-of-cleavage in a cell-state specific manner. For example, various cell states (e.g., following cellular signaling, such as immune cell activation) can influence the expression and/or localization of certain proteases. As an illustrative example, ADAM17 protein levels and localization is known to be influenced by signaling, such as through Protein kinase C (PKC) signaling pathways (e.g., activation by the PKC activator Phorbol-12-myristat-13-acetat [PMA]). Accordingly, a protease cleavage site can be selected and/or engineered such that cleavage of the protease cleavage site and subsequent release of an effector molecule is increased or decreased, as desired, depending on the protease properties (e.g., expression and/or localization) in a specific cell state. As another example, a protease cleavage site (particularly in combination with a specific membrane tethering domain) can be selected and/or engineered for optimal protein expression of the chimeric protein.

### Cell Membrane Tethering Domain

The membrane-cleavable chimeric proteins provided for herein contain a cell-membrane tethering domain (referred to as “MT” in the formula S—C-MT or MT-C—S). In general, the cell-membrane tethering domain can be any amino acid sequence motif capable of directing the chimeric protein to be localized to (e.g., inserted into), or otherwise associated with, the cell membrane of the cell expressing the chimeric protein. The cell-membrane tethering domain can be a transmembrane-intracellular domain. The cell-membrane tethering domain can be a transmembrane domain. The cell-membrane tethering domain can be an integral membrane protein domain (e.g., a transmembrane domain). The cell-membrane tethering domain can be derived from a Type I, Type II, or Type III transmembrane protein. The cell-membrane tethering domain can include post-translational modification tag, or motif capable of post-translational modification to modify the chimeric protein to include a post-translational modification tag, where the post-translational modification tag allows association with a cell membrane. Examples of post-translational modification tags include, but are not limited to, lipid-anchor domains (e.g., a GPI lipid-anchor, a myristoylation tag, or palmitoylation tag). Examples of cell-membrane tethering domains include, but are not limited to, a transmembrane-intracellular domain and/or transmembrane domain derived from PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, or BTLA. The cell membrane tethering domain can be a cell surface receptor or a cell membrane-bound portion thereof.

In some embodiments, the cell-membrane tethering domain comprises a transmembrane domain derived from a B7-1 polypeptide. In some embodiments, the B7-1 transmembrane domain comprises the sequence LLPSWAITLISVNGIFVICCLTYCFAPRCRERRRNERLRRESVRPV (SEQ ID NO: 204). In some embodiments, the B7-1 transmembrane domain is encoded by a polynucleotide sequence having the sequence TTGCTGCCTAGCTGGGCCATCACACTGATCTCCGTGAACGGCATCTTCGTGATCTGC TGCCTGACCTACTGCTTCGCCCCTAGATGCAGAGAGCGGAGAAGAAACGAGCGGCT GAGAAGAGAAAGCGTGCGGCCTGTG (SEQ ID NO: 252). In some embodiments, the B7-1 transmembrane domain is encoded by a polynucleotide sequence that is at least 90%, at least 91%, at least 92%, at least 93%, at least 94%, at least 95%, at least 96%, at least 97%, at least 98%, or at least 99% identical to

(SEQ ID NO: 252)
TTGCTGCCTAGCTGGGCCATCACACTGATCTCCGTGAACGGCATC
TTCGTGATCTGCTGCCTGACCTACTGCTTCGCCCCTAGATGCAGA
GAGCGGAGAAGAAACGAGCGGCTGAGAAGAGAAAGCGTGCGGCCT
GTG.

In some embodiments, the cell-membrane tethering domain of the membrane-cleavable chimeric protein (e.g., IL-15) includes a transmembrane domain derived from a B7-1 polypeptide. In some embodiments, the B7-1 transmembrane domain of the membrane-cleavable chimeric protein (e.g., IL-15) includes the sequence LLPSWAITLISVNGIFVICCLTYCFAPRCRERRRNERLRRESVRPV (SEQ ID NO: 204). In some embodiments, the B7-1 transmembrane domain of the membrane-cleavable chimeric protein (e.g., IL-15) is encoded by a polynucleotide sequence having the sequence TTGCTGCCTAGCTGGGCCATCACACTGATCTCCGTGAACGGCATCTTCGTGATCTGC TGCCTGACCTACTGCTTCGCCCCTAGATGCAGAGAGCGGAGAAGAAACGAGCGGCT GAGAAGAGAAAGCGTGCGGCCTGTG (SEQ ID NO: 252). In some embodiments, the B7-1 transmembrane domain of the membrane-cleavable chimeric protein (e.g., IL-15) is encoded by a polynucleotide sequence that is at least 90%, at least 91%, at least 92%, at least 93%, at least 94%, at least 95%, at least 96%, at least 97%, at least 98%, or at least 99% identical to

(SEQ ID NO: 252)
TTGCTGCCTAGCTGGGCCATCACACTGATCTCCGTGAACGGCATC
TTCGTGATCTGCTGCCTGACCTACTGCTTCGCCCCTAGATGCAGA
GAGCGGAGAAGAAACGAGCGGCTGAGAAGAGAAAGCGTGCGGCCT
GTG.

In some embodiments, the cell-membrane tethering domain comprises a transmembrane domain derived from a CD8 polypeptide. Any suitable CD8 polypeptide may be used. Exemplary CD8 polypeptides include, without limitation, NCBI Reference Nos. NP_001139345 and AAA92533.1. Examples of CD8 transmembrane domains include IYIWAPLAGTCGVLLLSLVIT (SEQ ID NO: 205), IYIWAPLAGTCGVLLLSLVITLYCNHR (SEQ ID NO: 206), and IYIWAPLAGTCGVLLLSLVITLYCNHRN (SEQ ID NO: 207). In some embodiments, the transmembrane domain comprises the sequence IYIWAPLAGTCGVLLLSLVIT (SEQ ID NO: 205). In some embodiments, the transmembrane domain comprises the sequence IYIWAPLAGTCGVLLLSLVITLYCNHR (SEQ ID NO: 206). In some embodiments, the transmembrane domain comprises the sequence IYIWAPLAGTCGVLLLSLVITLYCNHRN (SEQ ID NO: 207). In some embodiments, the cell-membrane tethering domain comprises a hinge and transmembrane domain derived from CD8. In some embodiments, the CD8 hinge comprises the sequence TTTPAPRPPTPAPTIALQPLSLRPEACRPAAGGAVHTRGLDFACD (SEQ ID NO: 271). In some embodiments, the CD8 hinge comprises the sequence AAAFVPVFLPAKPTTTPAPRPPTPAPTIASQPLSLRPEACRPAAGGAVHTRGLDFACDIYI WAPLAGTCGVLLLSLVITLYCNHRN (SEQ ID NO: 209).

In general, for all membrane-cleavable chimeric proteins described herein, the cell membrane tethering domain is either: (1)C-terminal of the protease cleavage site and N-terminal of any intracellular domain, if present (in other words, the cell membrane tethering domain is in between the protease cleavage site and, if present, an intracellular domain); or (2) N-terminal of the protease cleavage site and C-terminal of any intracellular domain, if present (also between the protease cleavage site and, if present, an intracellular domain with domain orientation inverted). In embodiments featuring a degron associated with the chimeric protein, the degron domain is the terminal cytoplasmic-oriented domain, specifically relative to the cell membrane tethering (in other words, the cell membrane tethering domain is in between the protease cleavage site and the degron). The cell membrane tethering domain can be connected to the protease cleavage site by a polypeptide linker, i.e., a polypeptide sequence not generally considered to be part of cell membrane tethering domain or protease cleavage site. The cell membrane tethering domain can be connected to an intracellular domain, if present, by a polypeptide linker, i.e., a polypeptide sequence not generally considered to be part of the cell membrane tethering domain or the intracellular domain. The cell membrane tethering domain can be connected to the degron, if present, by a polypeptide linker, i.e., a polypeptide sequence not generally considered to be part of the cell membrane tethering domain or degron. A polypeptide linker can be any amino acid sequence that connects a first polypeptide sequence and a second polypeptide sequence. A polypeptide linker can be a flexible linker (e.g., a Gly-Ser-Gly sequence). Examples of polypeptide linkers include, but are not limited to, GSG linkers (e.g., [GS]₄GG [SEQ ID NO: 504]), A(EAAAK)₃A (SEQ ID NO: 505), and Whitlow linkers (e.g., a “KEGS” (SEQ ID NO: 506) linker such as the amino acid sequence KESGSVSSEQLAQFRSLD (SEQ ID NO: 184), an eGK linker such as the amino acid sequence EGKSSGSGSESKST (SEQ ID NO: 185), and linkers described in more detail in Issued U.S. Pat. No. 5,990,275 herein incorporated by reference). Additional polypeptide linkers include SEQ ID NO: 194, SEQ ID NO: 195, SEQ ID NO: 196, and SEQ ID NO: 197. Other polypeptide linkers may be selected based on desired properties (e.g., length, flexibility, amino acid composition etc.) and are known to those skilled in the art.

In general, the cell-membrane tethering domain is oriented such that the secreted effector molecule and the protease cleavage site are extracellularly exposed following insertion into, or association with, the cell membrane, such that the protease cleavage site is capable of being cleaved by its respective protease and releasing (“secreting”) the effector molecule into the extracellular space.

### Degron Systems and Domains

In some embodiments, any of the proteins described herein can include a degron domain including, but not limited to, a protease, a transcription factor, a promoter or constituent of a promoter system (e.g., an ACP), and/or any of the membrane-cleavable chimeric protein described herein. In general, the degron domain can be any amino acid sequence motif capable of directing regulated degradation, such as regulated degradation through a ubiquitin-mediated pathway. In the presence of an immunomodulatory drug (IMiD), the degron domain directs ubiquitin-mediated degradation of a degron-fusion protein.

The degron domain can be a cereblon (CRBN) polypeptide substrate domain capable of binding CRBN in response to an immunomodulatory drug (IMiD) including, but not limited to, IKZF1, IKZF3, CK1a, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN. The CRBN polypeptide substrate domain can be a chimeric fusion product of native CRBN polypeptide sequences, such as a IKZF3/ZFP91/IKZF3 chimeric fusion product having the amino acid sequence of FNVLMVHKRSHTGERPLQCEICGFTCRQKGNLLRHIKLHTGEKPFKCHLCNYACQRRD AL (SEQ ID NO: 175). Degron domains, and in particular CRBN degron systems, are described in more detail in International Application Pub. No. WO2019/089592A1, herein incorporated by reference for all purposes. Other examples of degron domains include, but are not limited to HCV NS4 degron, PEST (two copies of residues 277-307 of human IκBα; SEQ ID NO: 161), GRR (residues 352-408 of human p105; SEQ ID NO: 162), DRR (residues 210-295 of yeast Cdc34; SEQ ID NO: 163), SNS (tandem repeat of SP2 and NB (SP2-NB-SP2 of influenza A or influenza B; e.g., SEQ ID NO: 164), RPB (four copies of residues 1688-1702 of yeast RPB; SEQ ID NO: 165), SPmix (tandem repeat of SP1 and SP2 (SP2-SP1-SP2-SP1-SP2 of influenza A virus M2 protein; SEQ ID NO: 166), NS2 (three copies of residues 79-93 of influenza A virus NS protein; SEQ ID NO: 167), ODC (residues 106-142 of ornithine decarboxylase; SEQ ID NO: 168), Nek2A, mouse ODC (residues 422-461; SEQ ID NO: 169), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS (SEQ ID NO: 509) phospho-dependent degron, an Siah binding motif, an SPOP SBC docking motif, or a PCNA binding PIP box.

Regulated degradation can be drug-inducible. Drugs capable of mediating/regulating degradation can be small-molecule compounds. Drugs capable of mediating/regulating degradation can include an “immunomodulatory drug” (IMiD). In general, as used herein, IMiDs refer to a class of small-molecule immunomodulatory drugs containing an imide group. Cereblon (CRBN) is known target of IMiDs and binding of an IMiD to CRBN or a CRBN polypeptide substrate domain alters the substrate specificity of the CRBN E3 ubiquitin ligase complex leading to degradation of proteins having a CRBN polypeptide substrate domain (e.g., any of secretable effector molecules or other proteins of interest described herein). For degron domains having a CRBN polypeptide substrate domain, examples of imide-containing IMiDs include, but are not limited to, a thalidomide, a lenalidomide, or a pomalidomide. The IMID can be an FDA-approved drug.

Chimeric proteins described herein can contain a degron domain (e.g., referred to as “D” in the formula S—C-MT-D or D-MT-C—S for membrane-cleavable chimeric proteins described herein). In the absence of an IMiD, degron/ubiquitin-mediated degradation of the chimeric protein does not occur. Following expression and localization of the chimeric protein into the cell membrane, the protease cleavage site directs cleavage of the chimeric protein such that the effector molecule is released (“secreted”) into the extracellular space. In the presence of an immunomodulatory drug (IMiD), the degron domain directs ubiquitin-mediated degradation of the chimeric protein such that secretion of the effector molecule is reduced or eliminated. In general, for membrane-cleavable chimeric proteins fused to a degron domain, the degron domain is the terminal cytoplasmic-oriented domain, specifically relative to the cell membrane tethering domain, e.g., the most C-terminal domain in the formula S—C-MT-D or the most N-terminal domain in the formula D-MT-C—S. The degron domain can be connected to the cell membrane tethering domain by a polypeptide linker, i.e., a polypeptide sequence not generally considered to be part of the cell membrane tethering domain or the degron domain. A polypeptide linker can be any amino acid sequence that connects a first polypeptide sequence and a second polypeptide sequence. A polypeptide linker can be a flexible linker (e.g., a Gly-Ser-Gly sequence). Examples of polypeptide linkers include, but are not limited to, GSG linkers (e.g., [GS]₄GG [SEQ ID NO: 504]), A(EAAAK)₃A (SEQ ID NO: 505), and Whitlow linkers (e.g., a “KEGS” (SEQ ID NO: 506) linker such as the amino acid sequence KESGSVSSEQLAQFRSLD (SEQ ID NO: 184), an eGK linker such as the amino acid sequence EGKSSGSGSESKST (SEQ ID NO: 185), and linkers described in more detail in Issued U.S. Pat. No. 5,990,275 herein incorporated by reference). Additional polypeptide linkers include SEQ ID NO: 194, SEQ ID NO: 195, SEQ ID NO: 196, and SEQ ID NO: 197. Other polypeptide linkers may be selected based on desired properties (e.g., length, flexibility, amino acid composition etc.) and are known to those skilled in the art. In general, the degron is oriented in relation to the cell membrane tethering domain such that the degron is exposed to the cytosol following localization to the cell membrane such that the degron domain is capable of mediating degradation (e.g., exposure to the cytosol and cytosol) and is capable of mediating ubiquitin-mediated degradation.

For degron-fusion proteins, the degron domain can be N-terminal or C-terminal of the protein of interest, e.g., the effector molecule. The degron domain can be connected to the protein of interest by a polypeptide linker, i.e., a polypeptide sequence not generally considered to be part of the protein of interest or the degron domain. A polypeptide linker can be any amino acid sequence that connects a first polypeptide sequence and a second polypeptide sequence. A polypeptide linker can be a flexible linker (e.g., a Gly-Ser-Gly sequence). Examples of polypeptide linkers include, but are not limited to, GSG linkers (e.g., [GS]₄GG [SEQ ID NO: 504], A(EAAAK)₃A (SEQ ID NO: 505), and Whitlow linkers (e.g., a “KEGS” (SEQ ID NO: 506) linker such as the amino acid sequence KESGSVSSEQLAQFRSLD (SEQ ID NO: 184), an eGK linker such as the amino acid sequence EGKSSGSGSESKST (SEQ ID NO: 185), and linkers described in more detail in Issued U.S. Pat. No. 5,990,275 herein incorporated by reference). Additional polypeptide linkers include SEQ ID NO: 194, SEQ ID NO: 195, SEQ ID NO: 196, and SEQ ID NO: 197. Other polypeptide linkers may be selected based on desired properties (e.g., length, flexibility, amino acid composition etc.) and are known to those skilled in the art. A polypeptide linker can be cleavable, e.g., any of the protease cleavage sites described herein.

### Homing Molecules

A “tumor microenvironment” is the cellular environment in which a tumor exists, including surrounding blood vessels, immune cells, fibroblasts, bone marrow-derived inflammatory cells, lymphocytes, signaling molecules and the extracellular matrix (ECM) (see, e.g., Pattabiraman, D. R. & Weinberg, R. A. Nature Reviews Drug Discovery 13, 497-512 (2014); Balkwill, F. R. et al. J Cell Sci 125, 5591-5596, 2012; and Li, H. et al. J Cell Biochem 101 (4), 805-15, 2007).

In some embodiments, engineered nucleic acids are configured to produce at least one homing molecule. For example, in membrane-cleavable chimeric proteins described herein containing a secreted effector molecule, the secreted effector molecule can be a homing molecule. “Homing,” refers to active navigation (migration) of a cell to a target site (e.g., a cell, tissue (e.g., tumor), or organ). A “homing molecule” refers to a molecule that directs cells to a target site. In some embodiments, a homing molecule functions to recognize and/or initiate interaction of an engineered cell to a target site. Non-limiting examples of homing molecules include CXCR1, CCR9, CXCR2, CXCR3, CXCR4, CCR2, CCR4, FPR2, VEGFR, IL6R, CXCR1, CSCR7, and PDGFR.

In some embodiments, a homing molecule is a chemokine receptor (cell surface molecule that binds to a chemokine). Chemokines are small cytokines or signaling proteins secreted by cells that can induce directed chemotaxis in cells. Chemokines can be classified into four main subfamilies: CXC, CC, CX3C and XC, all of which exert biological effects by binding selectively to chemokine receptors located on the surface of target cells. In some embodiments, engineered nucleic acids are configured to produce CXCR4, a chemokine receptor which allows engineered cells to home along a chemokine gradient towards a stromal cell-derived factor 1 (also known as SDF1, C—X—C motif chemokine 12, and CXCL12)-expressing cell, tissue, or tumor. Non-limiting examples of chemokine receptors that may be encoded by the engineered nucleic acids of the present disclosure include: CXC chemokine receptors (e.g., CXCR1, CXCR2, CXCR3, CXCR4, CXCR5, CXCR6, and CXCR7), CC chemokine receptors (CCR1, CCR2, CCR3, CCR4, CCR5, CCR6, CCR7, CCR8, CCR9, CCR10, and CCR11), CX3C chemokine receptors (e.g., CX3CR1, which binds to CX3CL1), and XC chemokine receptors (e.g., XCR1). In some embodiments, a chemokine receptor is a G protein-linked transmembrane receptor, or a member of the tumor necrosis factor (TNF) receptor superfamily (including but not limited to TNFRSF1A, TNFRSF1B). In some embodiments, engineered nucleic acids are configured to produce CXCL8, CXCL9, and/or CXCL10 (promote T-cell recruitment), CCL3 and/or CXCL5, CCL21 (Th1 recruitment and polarization).

In some embodiments, engineered nucleic acids are configured to produce G-protein coupled receptors (GPCRs) that detect N-formylated-containing oligopeptides (including but not limited to FPR2 and FPRL1).

In some embodiments, engineered nucleic acids are configured to produce receptors that detect interleukins (including but not limited to IL6R).

In some embodiments, engineered nucleic acids are configured to produce receptors that detect growth factors secreted from other cells, tissues, or tumors (including but not limited to FGFR, PDGFR, EGFR, and receptors of the VEGF family, including but not limited to VEGF-C and VEGF-D).

In some embodiments, a homing molecule is an integrin. Integrins are transmembrane receptors that facilitate cell-extracellular matrix (ECM) adhesion. Integrins are obligate heterodimers having two subunits: α (alpha) and β (beta). The a subunit of an integrin may be, without limitation: ITGA1, ITGA2, ITGA3, ITGA4, ITGA5, ITGA6, IGTA7, ITGA8, ITGA9, IGTA10, IGTA11, ITGAD, ITGAE, ITGAL, ITGAM, ITGAV, ITGA2B, ITGAX. The β subunit of an integrin may be, without limitation: ITGB1, ITGB2, ITGB3, ITGB4, ITGB5, ITGB6, ITGB7, and ITGB8. Engineered nucleic acids can be configured to produce any combination of the integrin α and β subunits.

In some embodiments, a homing molecule is a matrix metalloproteinase (MMP). MMPs are enzymes that cleave components of the basement membrane underlying the endothelial cell wall. Non-limiting examples of MMPs include MMP-2, MMP-9, and MMP. In some embodiments, engineered nucleic acids are configured to produce an inhibitor of a molecule (e.g., protein) that inhibits MMPs. For example, engineered nucleic acids can be configured to express an inhibitor (e.g., an RNAi molecule) of membrane type 1 MMP (MT1-MMP) or TIMP metallopeptidase inhibitor 1 (TIMP-1).

In some embodiments, a homing molecule is a ligand that binds to selectin (e.g., hematopoietic cell E-/L-selectin ligand (HCELL), Dykstran et al., Stem Cells. 2016 October; 34 (10): 2501-2511) on the endothelium of a target tissue, for example.

The term “homing molecule” also encompasses transcription factors that regulate the production of molecules that improve/enhance homing of cells.

In some embodiments, a homing molecule includes an antibody, such as anti-integrin alpha4,beta7 or anti-MAdCAM.

### Chimeric Antigen Receptors (CARs)

Certain aspects of the present disclosure relate to chimeric receptors that have any one of the antigen-specific binding domains described herein (e.g., EMCN-specific, FLT3-specific, and/or CD33-specific) and are capable of specifically binding to a protein, an antigen-derived antigen, or an antigen-derived epitope.

In some embodiments, the chimeric receptor is a chimeric antigen receptor (CAR). In general, CARs are chimeric proteins that include an antigen-binding domain and polypeptide molecules that are heterologous to the antigen-binding domain, such as peptides heterologous to an antibody that an antigen-binding domain may be derived from. Polypeptide molecules that are heterologous to the antigen-binding domain can include, but are not limited to, a transmembrane domain, one or more intracellular signaling domains, a hinge domain, a spacer region, one or more peptide linkers, or combinations thereof.

In some embodiments, CARs are engineered receptors that graft or confer a specificity of interest (e.g., EMCN, FLT3, or CD33) onto an immune effector cell. In certain embodiments, CARs can be used to graft the specificity of an antibody onto an immunoresponsive cell, such as a T cell. In some embodiments, CARs of the present disclosure comprise an extracellular antigen-binding domain (e.g., an scFv) fused to a transmembrane domain, fused to one or more intracellular signaling domains.

In some embodiments, the chimeric antigen receptor is an activating chimeric antigen receptor (aCAR and also generally referred to as CAR unless otherwise specified). In some embodiments, binding of the chimeric antigen receptor to its cognate ligand is sufficient to induce activation of the immunoresponsive cell. In some embodiments, binding of the chimeric antigen receptor to its cognate ligand is sufficient to induce stimulation of the immunoresponsive cell. In some embodiments, activation of an immunoresponsive cell results in killing of target cells. In some embodiments, activation of an immunoresponsive cell results in cytokine or chemokine expression and/or secretion by the immunoresponsive cell. In some embodiments, stimulation of an immunoresponsive cell results in cytokine or chemokine expression and/or secretion by the immunoresponsive cell. In some embodiments, stimulation of an immunoresponsive cell induces differentiation of the immunoresponsive cell. In some embodiments, stimulation of an immunoresponsive cell induces proliferation of the immunoresponsive cell. In some embodiments, activation and/or stimulation of the immunoresponsive cell can be combinations of the above responses.

The number of ABDs in a binding molecule, such as the chimeric proteins described herein, defines the “valency” of the binding molecule. A binding molecule having a single ABD is “monovalent”. A binding molecule having a plurality of ABDs is said to be “multivalent”. A multivalent binding molecule having two ABDs is “bivalent.” A multivalent binding molecule having three ABDs is “trivalent.” A multivalent binding molecule having four ABDs is “tetravalent.” In various multivalent embodiments, all of the plurality of ABDs have the same recognition specificity and can be referred to as a “monospecific multivalent” binding molecule. In other multivalent embodiments, at least two of the plurality of ABDs have different recognition specificities. Such binding molecules are multivalent and “multispecific.” In multivalent embodiments in which the ABDs collectively have two recognition specificities, the binding molecule is “bispecific.” In multivalent embodiments in which the ABDs collectively have three recognition specificities, the binding molecule is “trispecific.” In multivalent embodiments in which the ABDs collectively have a plurality of recognition specificities for different epitopes present on the same antigen, the binding molecule is “multiparatopic.” Multivalent embodiments in which the ABDs collectively recognize two epitopes on the same antigen are “biparatopic.”

In various multivalent embodiments, multivalency of the binding molecule improves the avidity of the binding molecule for a specific target. As described herein, “avidity” refers to the overall strength of interaction between two or more molecules, e.g. a multivalent binding molecule for a specific target, wherein the avidity is the cumulative strength of interaction provided by the affinities of multiple ABDs. Avidity can be measured by the same methods as those used to determine affinity, as described above. In certain embodiments, the avidity of a binding molecule for a specific target is such that the interaction is a specific binding interaction, wherein the avidity between two molecules has a KD value below 10⁻⁶M, 10⁻⁷M, 10⁻⁸M, 10⁻⁹M, or 10⁻¹⁰M. In certain embodiments, the avidity of a binding molecule for a specific target has a KD value such that the interaction is a specific binding interaction, wherein the one or more affinities of individual ABDs do not have has a KD value that qualifies as specifically binding their respective antigens or epitopes on their own. In certain embodiments, the avidity is the cumulative strength of interaction provided by the affinities of multiple ABDs for separate antigens on a shared specific target or complex, such as separate antigens found on an individual cell. In certain embodiments, the avidity is the cumulative strength of interaction provided by the affinities of multiple ABDs for separate epitopes on a shared individual antigen.

In some embodiments, an aCAR can be a bivalent-bispecific CAR. In some embodiments, an aCAR can be a bivalent-bispecific CAR expressed on a cell of the present disclosure (e.g., an immunoresponsive cell) as OR-logic gates to increase the potential targets of an activating chimeric receptor (e.g., an OR-logic gate targeting multiple tumor targets). A bivalent-bispecific aCAR can include an antigen-binding domain specific for FLT3 and an antigen-binding domain specific for CD33. A bivalent-bispecific aCAR can include i) an antigen-binding domain specific for FLT3; (ii) an antigen-binding domain specific for CD33; (iii) one or more intracellular signaling domains that stimulate an immune response, and (iv) one or more polypeptides including, but not limited to, a signal peptide, a transmembrane domain, a hinge domain, a spacer region, one or more peptide linkers, and combinations thereof.

A CAR of the present disclosure may be a first, second, or third generation CAR. “First generation” CARs comprise a single intracellular signaling domain, generally derived from a T cell receptor chain. “First generation” CARs generally have the intracellular signaling domain from the CD3-zeta (CD3ζ) chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add a second intracellular signaling domain from one of various co-stimulatory molecules (e.g., CD28, 4-1BB, ICOS, OX40) to the cytoplasmic tail of the CAR to provide additional signals to the T cell. “Second generation” CARs provide both co-stimulation (e.g., CD28 or 4-1BB) and activation (CD35). Preclinical studies have indicated that “Second Generation” CARs can improve the anti-tumor activity of immunoresponsive cell, such as a T cell. “Third generation” CARs have multiple intracellular co-stimulation signaling domains (e.g., CD28 and 4-1BB) and an intracellular activation signaling domain (CD3ζ).

In some embodiments, the chimeric antigen receptor is a chimeric inhibitory receptor (iCAR). In some embodiments, the one or more chimeric inhibitory receptors bind antigens that are expressed on a non-tumor cell derived from a tissue selected from the group consisting of brain, neuronal tissue, endocrine, bone, bone marrow, immune system, endothelial tissue, muscle, lung, liver, gallbladder, pancreas, gastrointestinal tract, kidney, urinary bladder, male reproductive organs, female reproductive organs, adipose, soft tissue, and skin.

In some embodiments, a chimeric inhibitory receptor (e.g., an EMCN-specific chimeric inhibitory receptor) may be used, for example, with one or more activating chimeric receptors (e.g., activating chimeric TCRs or CARs, such as FLT3 and/or CD33 aCARs) expressed on a cell of the present disclosure (e.g., an immunoresponsive cell) as NOT-logic gates to control, modulate, or otherwise inhibit one or more activities of the one or more activating chimeric receptors. For instance, if a healthy cell expresses both an antigen that is recognized by a tumor-targeting chimeric receptor and an antigen that is recognized by an chimeric inhibitory receptor, an immunoresponsive cell expressing the tumor antigen may bind to the healthy cell. In such a case, the inhibitory chimeric antigen will also bind its cognate ligand on the healthy cell and the inhibitory function of the chimeric inhibitory receptor will reduce, decrease, prevent, or inhibit the activation of the immunoresponsive cell via the tumor-targeting chimeric receptor (“NOT-logic gating”). In some embodiments, a chimeric inhibitory receptor of the present disclosure may inhibit one or more activities of a cell of the present disclosure (e.g., an immunoresponsive cell). In some embodiments, an immunoresponsive cell may comprise one or more tumor-targeting chimeric receptors and one or more chimeric inhibitory receptors that targets an antigen that is not expressed, or generally considered to be expressed, on the tumor (e.g., EMCN). Combinations of tumor-targeting chimeric receptors and chimeric inhibitory receptors in the same immunoresponsive cell may be used to reduce on-target off-tumor toxicity.

In some embodiments, the extracellular antigen-binding domain of a CAR of the present disclosure binds to one or more antigens (e.g., EMCN, FLT3, or CD33) with a dissociation constant (K_{d}) of about 2×10⁻⁷ M or less, about 1×10⁻⁷M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ M or less, about 2×10⁻⁹ M or less, or about 1×10⁻⁹ M or less. In some embodiments, the K_{d} ranges from about is about 2×10⁻⁷ M to about 1×10⁻⁹ M.

Binding of the extracellular antigen-binding domain of a CAR of the present disclosure can be determined by, for example, an enzyme-linked immunosorbent assay (ELISA), a radioimmunoassay (RIA), FACS analysis, a bioassay (e.g., growth inhibition), bio-layer interferometry (e.g., Octet/FORTEBIO®), surface plasmon resonance (SPR) technology (e.g., Biacore®), or a Western Blot assay. Each of these assays generally detect the presence of protein-antibody complexes of particular interest by employing a labeled reagent (e.g., an antibody or scFv) specific for the complex of interest. For example, the scFv can be radioactively labeled and used in an RIA assay. The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPct), and yellow fluorescent protein (e.g., YFP, Citrine, Venus, and YPet). In certain embodiments, the extracellular antigen-binding domain of the CAR is labeled with a secondary antibody specific for the extracellular antigen-binding domain and wherein the secondary antibody is labeled (e.g., radioactively or with a fluorescent marker).

In some embodiments, CARs of the present disclosure comprise an extracellular antigen-binding domain that binds to EMCN, FLT3, or CD33. In some embodiments, CARs of the present disclosure comprise an extracellular antigen-binding domain that binds to an EMCN protein, an EMCN-derived antigen, or an EMCN-derived epitope. In some embodiments, CARs of the present disclosure comprise an extracellular antigen-binding domain that binds to a FLT3 protein, a FLT3-derived antigen, or a FLT3-derived epitope. In some embodiments. In some embodiments, CARs of the present disclosure comprise an extracellular antigen-binding domain that binds to an CD33 protein, a CD33-derived antigen, or a CD33-derived epitope. CARs of the present disclosure comprise an extracellular antigen-binding domain, a transmembrane domain, and one or more intracellular signaling domains. In some embodiments, the extracellular antigen-binding domain comprises an scFv. In some embodiments, the extracellular antigen-binding domain comprises a Fab fragment, which may be crosslinked. In certain embodiments, the extracellular binding domain is a F(ab)₂ fragment

### Extracellular Antigen-Binding Domain

Antigen-binding domains of the present disclosure can include any domain that binds to the antigen including, without limitation, a monoclonal antibody, a polyclonal antibody, a recombinant antibody, a bispecific antibody, a conjugated antibody, a human antibody, a humanized antibody, and a functional fragment thereof, including but not limited to a single-domain antibody (sdAb) such as a heavy chain variable domain (VH), a light chain variable domain (VL) and a variable domain (VHH) of camelid derived nanobody, and to an alternative scaffold known in the art to function as antigen-binding domain, such as a recombinant fibronectin domain, a T cell receptor (TCR), a recombinant TCR with enhanced affinity, or a fragment thereof, e.g., single chain TCR, and the like. In some instances, it is beneficial for the antigen-binding domain to be derived from the same species in which the CAR will ultimately be used in.

In some embodiments, the extracellular antigen-binding domain comprises an antibody. In certain embodiments, the antibody is a human antibody. In certain embodiments, the antibody is a chimeric antibody. In some embodiments, the extracellular antigen-binding domain comprises an antigen-binding fragment of an antibody.

In some embodiments, the extracellular antigen-binding domain comprises a F(ab) fragment. In certain embodiments, the extracellular antigen-binding domain comprises a F(ab′) fragment.

In some embodiments, the extracellular antigen-binding domain comprises an scFv. In some embodiments, the extracellular antigen-binding domain comprises two single chain variable fragments (scFvs). In some embodiments, each of the two scFvs binds to a distinct epitope on the same antigen. In some embodiments, the extracellular antigen-binding domain comprises a first scFv and a second scFv. In some embodiments, the first scFv and the second scFv bind distinct epitopes on the same antigen. In certain embodiments, the scFv is a mammalian scFv. In certain embodiments, the scFv is a chimeric scFv. In certain embodiments, the scFv comprises a heavy chain variable domain (VH) and a light chain variable domain (VL).

In certain embodiments, the VH and VL are separated by a peptide linker. In certain embodiments, the peptide linker comprises any of the amino acid sequences shown in Table 6.

In certain embodiments, the scFv comprises the structure VH-L-VL or VL-L-VH, wherein VH is the heavy chain variable domain, L is the peptide linker, and VL is the light chain variable domain. In some embodiments, each of the one or more scFvs comprises the structure VH-L-VL or VL-L-VH, wherein VH is the heavy chain variable domain, L is the peptide linker, and VL is the light chain variable domain. When there are two or more scFv linked together, each scFv can be linked to the next scFv with a peptide linked. In some embodiments, each of the one or more scFvs is separated by a peptide linker.

In some embodiments, a peptide linker includes the amino acid sequence GGGGSGGGGSGGGGS (SEQ ID NO: 244). In some embodiments, a peptide linker between antigen binding domains of an iCAR comprises the amino acid sequence GGGGGGGGSGGGGS (SEQ ID NO: 244). In some embodiments, a peptide linker is encoded by a polynucleotide sequence comprising the sequence GGAGGCGGAGGATCTGGTGGCGGAGGAAGTGGCGGAGGCGGTTCT (SEQ ID NO: 253). In some embodiments, a nucleic acid encoding peptide linker includes a sequence that is at least 90%, at least 91%, at least 92%, at least 93%, at least 94%, at least 95%, at least 96%, at least 97%, at least 98%, or at least 99% identical to

(SEQ ID NO: 253)
GGAGGCGGAGGATCTGGTGGCGGAGGAAGTGGCGGAGGCGGTTCT.

In some embodiments, a peptide linker includes the amino acid sequence GGGGS (SEQ ID NO: 242). In some embodiments, a peptide linker between antigen binding domains of an aCAR comprises the amino acid sequence GGGGS (SEQ ID NO: 242). In some embodiments, a peptide linker is encoded by a polynucleotide sequence comprising the sequence GGCGGCGGTGGCTCT (SEQ ID NO: 254) or GGTGGCGGCGGATCC (SEQ ID NO: 255). In some embodiments, a nucleic acid encoding peptide linker includes a sequence that is at least 90%, at least 91%, at least 92%, at least 93%, at least 94%, at least 95%, at least 96%, at least 97%, at least 98%, or at least 99% identical to GGCGGCGGTGGCTCT (SEQ ID NO: 254) or GGTGGCGGCGGATCC (SEQ ID NO: 255).

In some embodiments, a peptide linker includes the amino acid sequence GGGGSGGGGS (SEQ ID NO: 243). In some embodiments, a peptide linker between antigen binding domains of an aCAR comprises the amino acid sequence GGGGSGGGGS (SEQ ID NO: 243). In some embodiments, a peptide linker is encoded by a polynucleotide sequence comprising the sequence GGAGGCGGAGGATCTGGTGGTGGTGGATCT (SEQ ID NO: 256), GGTGGCGGAGGAAGTGGCGGCGGAGGCTCT (SEQ ID NO: 257), or GGCGGTGGCGGATCTGGCGGAGGTGGCAGT (SEQ ID NO: 258). In some embodiments, a nucleic acid encoding peptide linker includes a sequence that is at least 90%, at least 91%, at least 92%, at least 93%, at least 94%, at least 95%, at least 96%, at least 97%, at least 98%, or at least 99% identical to GGAGGCGGAGGATCTGGTGGTGGTGGATCT (SEQ ID NO: 256), GGTGGCGGAGGAAGTGGCGGCGGAGGCTCT (SEQ ID NO: 257), or GGCGGTGGCGGATCTGGCGGAGGTGGCAGT (SEQ ID NO: 258).

In some embodiments, a peptide linker includes the amino acid sequence GSTSGSGKPGSGEGSTKG (SEQ ID NO: 247). In some embodiments, a peptide linker between antigen binding domains of an aCAR comprises the amino acid sequence GSTSGSGKPGSGEGSTKG (SEQ ID NO: 247). In some embodiments, a peptide linker is encoded by a polynucleotide sequence comprising the sequence GGCTCTACATCTGGCTCTGGCAAACCTGGAAGCGGCGAGGGATCTACCAAGGGC (SEQ ID NO: 249). In some embodiments, a nucleic acid encoding peptide linker includes a sequence that is at least 90%, at least 91%, at least 92%, at least 93%, at least 94%, at least 95%, at least 96%, at least 97%, at least 98%, or at least 99% identical to

(SEQ ID NO: 249)
GGCTCTACATCTGGCTCTGGCAAACCTGGAAGCGGCGAGGGATCT
ACCAAGGGC.

TABLE 6
Peptide Linkers

| | | SEQ ID |
|---|---|---|
| Linker | Amino Acid Sequence | NO: |
| GS-linker | SGGGGSGGGGSG | 230 |
| GS-linker | GGGSGGGGSGGGSLQ | 231 |
| (G₂S)₁ scFv linker | GGS | 232 |
| (G₂S)₂ scFv linker | GGSGGS | 233 |
| (G₂S)₃ scFv linker | GGSGGSGGS | 234 |
| (G₂S)₄ scFv linker | GGSGGSGGSGGS | 235 |
| (G₂S)₅ scFv linker | GGSGGSGGSGGSGGS | 236 |
| (G₃S)₁ scFv linker | GGGS | 237 |
| (G₃S)₂ scFv linker | GGGSGGGS | 238 |
| (G₃S)₃ scFv linker | GGGSGGGSGGGS | 239 |
| (G₃S)₄ scFv linker | GGGSGGGSGGGSGGGS | 240 |
| (G₃S)₅ scFv linker | GGGSGGGSGGGSGGGSGGGS | 241 |
| (G₄S)₁ linker | GGGGS | 242 |
| (G₄S)₂ linker | GGGGSGGGGS | 243 |
| (G₄S)₃ linker | GGGGSGGGGSGGGGS | 244 |
| (G₄S)₄ linker | GGGGSGGGGSGGGGSGGGGS | 245 |
| (G₄S)₅ linker | GGGGSGGGGSGGGGSGGGGSGGGGS | 246 |
| Whitlow linker | GSTSGSGKPGSGEGSTKG | 247 |
| [“Loop 6” linker] | | |
| scFv linker 2 | EAAAKEAAAKEAAAKEAAAK | 248 |

In some embodiments, the immune effector cell comprises a first chimeric receptor and a second chimeric receptor. The antigen-binding domain of the first chimeric receptor and the antigen-binding domain of the second chimeric receptor can be an appropriate antigen biding domain described herein or known in the art. For example, the first or second antigen-binding domain can be one or more antibodies, antigen-binding fragments of an antibody, F(ab) fragments, F(ab′) fragments, single chain variable fragments (scFvs), or single-domain antibodies (sdAbs). In some embodiments, the antigen-binding domain of the first chimeric receptor and/or the second chimeric receptor comprises two single chain variable fragments (scFvs). In some embodiments, each of the two scFvs binds to a distinct epitope on the same antigen. In some embodiments, the antigen-binding domain of the first chimeric receptor can be specific for EMCN and the chimeric receptor can be specific for a second distinct antigen, such as a cancer antigen (e.g., an antigen expressed on a myeloid cell, such as an AML cell).

In some embodiments, the extracellular antigen-binding domain comprises a single-domain antibody (sdAb). In certain embodiments, the sdAb is a humanized sdAb. In certain embodiments, the sdAb is a chimeric sdAb.

In some embodiments, a CAR of the present disclosure may comprise two or more antigen-binding domains, three or more antigen-binding domains, four or more antigen-binding domains, five or more antigen-binding domains, six or more antigen-binding domains, seven or more antigen-binding domains, eight or more antigen-binding domains, nine or more antigen-binding domains, or ten or more antigen-binding domains. In some embodiments, each of the two or more antigen-binding domains binds the same antigen. In some embodiments, each of the two or more antigen-binding domains binds a different epitope of the same antigen. In some embodiments, each of the two or more antigen-binding domains binds a different antigen.

In some embodiments, the CAR comprises two antigen-binding domains. In some embodiments, the two antigen-binding domains are attached to one another via a flexible linker. In some embodiments, each of the two-antigen-binding domains may be independently selected from an antibody, an antigen-binding fragment of an antibody, an scFv, a sdAb, a recombinant fibronectin domain, a T cell receptor (TCR), a recombinant TCR with enhanced affinity, and a single chain TCR. In some embodiments, the CAR comprising two antigen-binding domains is a bispecific CAR or a tandem CAR (tanCAR).

In certain embodiments, the bispecific CAR or tanCAR comprises an antigen-binding domain comprising a bispecific antibody or antibody fragment (e.g., scFv). In some embodiments, within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁-VL₁-VL₂-VH₂. In other embodiments, the upstream antibody or antibody fragment (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific antibody molecule has the arrangement VL₁ VH₁-VH₂-VL₂. In some embodiments, a linker is disposed between the two antibodies or antibody fragments (e.g., scFvs), for example, between VL and VL₂ if the construct is arranged as VH₁-VL₁-VL₂-VH₂, or between VH₁ and VH₂ if the construct is arranged as VL₁-VH₁-VH₂-VL₂. The linker may be a linker as described herein, e.g., a (Gly₄-Ser) n linker, wherein n is 1, 2, 3, 4, 5, or 6. In general, the linker between the two scFvs should be long enough to avoid mispairing between the domains of the two scFvs. In some embodiments, a linker is disposed between the VL and VH of the first scFv. In some embodiments, a linker is disposed between the VL and VH of the second scFv. In constructs that have multiple linkers, any two or more of the linkers may be the same or different. Accordingly, in some embodiments, a bispecific CAR or tanCAR comprises VLs, VHs, and may further comprise one or more linkers in an arrangement as described herein.

In an illustrative non-limiting example, a CD33/FLT3 bispecific bivalent aCAR can have antigen binding domains encoded in the following order: (FLT3-VH)-L1-(CD33-VH)-L2-(CD33-VL)-L3-(FLT3-VL), wherein L1, L2, and L3 are a first, a second, and a third peptide linker, respectively. L1, L2, and L3 can be the same peptide linker. L1, L2, and L3 can be the same peptide linker encoded by the same polynucleotide sequence. L1, L2, and L3 can be the same peptide linker encoded by different polynucleotide sequences. L1 and L3 can be the same peptide linker and L2 a different peptide linker. L1 and L3 can be the same peptide linker encoded by the same polynucleotide sequence. L1 and L3 can be the same peptide linker encoded by different polynucleotide sequences. L1, L2, and L3 can each be different peptide linkers.

### CAR Transmembrane Domains

In some embodiments, the transmembrane domain of a CAR of the present disclosure (e.g., the EMCN-specific, FLT3-specific, and/or CD33-specific CARs described herein) comprises a hydrophobic alpha helix that spans at least a portion of a cell membrane. It has been shown that different transmembrane domains can result in different receptor stability. After antigen recognition, receptors cluster and a signal is transmitted to the cell. In some embodiments, the transmembrane domain of a CAR of the present disclosure can comprise the transmembrane domain of a CD8 polypeptide, a CD28 polypeptide, a CD3-zeta polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a LIR-1 (LILRB1) polypeptide, or can be a synthetic peptide, or any combination thereof.

In some embodiments, the transmembrane domain is derived from a CD8 polypeptide. Any suitable CD8 polypeptide may be used. Exemplary CD8 polypeptides include, without limitation, NCBI Reference Nos. NP_001139345 and AAA92533.1. In some embodiments, the transmembrane domain is derived from a CD28 polypeptide. Any suitable CD28 polypeptide may be used. Exemplary CD28 polypeptides include, without limitation, NCBI Reference Nos. NP_006130.1 and NP_031668.3. In some embodiments, the transmembrane domain is derived from a CD3-zeta polypeptide. Any suitable CD3-zeta polypeptide may be used. Exemplary CD3-zeta polypeptides include, without limitation, NCBI Reference Nos. NP_932170.1 and NP_001106862.1. In some embodiments, the transmembrane domain is derived from a CD4 polypeptide. Any suitable CD4 polypeptide may be used. Exemplary CD4 polypeptides include, without limitation, NCBI Reference Nos. NP_000607.1 and NP_038516.1. In some embodiments, the transmembrane domain is derived from a 4-1BB polypeptide. Any suitable 4-1BB polypeptide may be used. Exemplary 4-1BB polypeptides include, without limitation, NCBI Reference Nos. NP_001552.2 and NP_001070977.1. In some embodiments, the transmembrane domain is derived from an OX40 polypeptide. Any suitable OX40 polypeptide may be used. Exemplary OX40 polypeptides include, without limitation, NCBI Reference Nos. NP_003318.1 and NP_035789.1. In some embodiments, the transmembrane domain is derived from an ICOS polypeptide. Any suitable ICOS polypeptide may be used. Exemplary ICOS polypeptides include, without limitation, NCBI Reference Nos. NP_036224 and NP_059508. In some embodiments, the transmembrane domain is derived from a CTLA-4 polypeptide. Any suitable CTLA-4 polypeptide may be used. Exemplary CTLA-4 polypeptides include, without limitation, NCBI Reference Nos. NP_005205.2 and NP_033973.2. In some embodiments, the transmembrane domain is derived from a PD-1 polypeptide. Any suitable PD-1 polypeptide may be used. Exemplary PD-1 polypeptides include, without limitation, NCBI Reference Nos. NP_005009 and NP_032824. In some embodiments, the transmembrane domain is derived from a LAG-3 polypeptide. Any suitable LAG-3 polypeptide may be used. Exemplary LAG-3 polypeptides include, without limitation, NCBI Reference Nos. NP_002277.4 and NP_032505.1. In some embodiments, the transmembrane domain is derived from a 2B4 polypeptide. Any suitable 2B4 polypeptide may be used. Exemplary 2B4 polypeptides include, without limitation, NCBI Reference Nos. NP_057466.1 and NP_061199.2. In some embodiments, the transmembrane domain is derived from a BTLA polypeptide. Any suitable BTLA polypeptide may be used. Exemplary BTLA polypeptides include, without limitation, NCBI Reference Nos. NP_861445.4 and NP_001032808.2. Any suitable LIR-1 (LILRB1) polypeptide may be used. Exemplary LIR-1 (LILRB1) polypeptides include, without limitation, NCBI Reference Nos. NP_001075106.2 and NP_001075107.2.

In some embodiments, the transmembrane domain comprises a polypeptide comprising an amino acid sequence that is at least 85%, at least 90%, at least 91%, at least 92%, at least 93%, at least 94%, at least 95%, at least 96%, at least 97%, at least 98%, at least 99%, or 100% homologous to the sequence of NCBI Reference No. NP_001139345, AAA92533.1, NP_006130.1, NP_031668.3, NP_932170.1, NP_001106862.1, NP_000607.1, NP_038516.1, NP_001552.2, NP_001070977.1, NP_003318.1, NP_035789.1, NP_036224, NP_059508, NP_005205.2, NP_033973.2, NP_005009, NP_032824, NP_002277.4, NP_032505.1, NP_057466.1, NP_061199.2, NP_861445.4, or NP_001032808.2, or fragments thereof. In some embodiments, the homology may be determined using standard software such as BLAST or FASTA. In some embodiments, the polypeptide may comprise one conservative amino acid substitution, up to two conservative amino acid substitutions, or up to three conservative amino acid substitutions. In some embodiments, the polypeptide can have an amino acid sequence that is a consecutive portion of NCBI Reference No. NP_001139345, AAA92533.1, NP_006130.1, NP_031668.3, NP_932170.1, NP_001106862.1, NP_000607.1, NP_038516.1, NP_001552.2, NP_001070977.1, NP_003318.1, NP_035789.1, NP_036224, NP_059508, NP_005205.2, NP_033973.2, NP_005009, NP_032824, NP_002277.4, NP_032505.1, NP_057466.1, NP_061199.2, NP_861445.4, or NP_001032808.2 that is at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, or at least 240 amino acids in length.

Further examples of suitable polypeptides from which a transmembrane domain may be derived include, without limitation, the transmembrane region(s) of the alpha, beta or zeta chain of the T-cell receptor, CD27, CD3 epsilon, CD45, CD5, CD9, CD16, CD22, CD33, CD37, CD64, CD80, CD86, CD134, CD137, CD154, KIRDS2, CD2, CD27, LFA-1 (CD11a, CD18), GITR, CD40, BAFFR, HVEM (LIGHTR), SLAMF7, NKp80 (KLRF1), NKp44, NKp30, NKp46, CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, and NG2C.

In some embodiments, the transmembrane domain comprises the sequence IYIWAPLAGTCGVLLLSLVIT (SEQ ID NO: 205). In some embodiments, the transmembrane domain comprises the sequence IYIWAPLAGTCGVLLLSLVITLYCNHR (SEQ ID NO: 206). In some embodiments, the transmembrane domain comprises the sequence IYIWAPLAGTCGVLLLSLVITLYCNHRN (SEQ ID NO: 207).

In some embodiments, an iCAR transmembrane domain includes a LIR1 transmembrane domain. In some embodiments, an iCAR transmembrane domain includes a LIR1 transmembrane domain having the sequence VIGILVAVILLLLLLLLLFLI (SEQ ID NO: 259). In some embodiments, an iCAR transmembrane domain includes a LIR1 transmembrane domain encoded by a polynucleotide sequence having the sequence GTGATCGGCATTCTGGTCGCCGTGATCCTGCTCCTGTTGCTCCTGCTGCTTCTGTTCC TGATC (SEQ ID NO: 260). In some embodiments, an iCAR transmembrane domain includes a LIR1 transmembrane domain encoded by a polynucleotide sequence that is at least 90%, at least 91%, at least 92%, at least 93%, at least 94%, at least 95%, at least 96%, at least 97%, at least 98%, or at least 99% identical to

(SEQ ID NO: 260)
GTGATCGGCATTCTGGTCGCCGTGATCCTGCTCCTGTTGCTCCTG
CTGCTTCTGTTCCTGATC.

In some embodiments, an aCAR transmembrane domain includes a CD8 transmembrane domain. In some embodiments, an aCAR transmembrane domain includes a CD8 transmembrane domain having the sequence IYIWAPLAGTCGVLLLSLVITLYCNHR (SEQ ID NO: 206). In some embodiments, an aCAR transmembrane domain includes a CD8 transmembrane domain encoded by a polynucleotide sequence having the sequence ATCTATATCTGGGCCCCTCTGGCTGGCACATGCGGAGTTCTGCTGCTCAGCCTGGTC ATCACCCTGTACTGCAACCACAGA (SEQ ID NO: 261). In some embodiments, an aCAR transmembrane domain includes a CD8 transmembrane domain encoded by a polynucleotide sequence that is at least 90%, at least 91%, at least 92%, at least 93%, at least 94%, at least 95%, at least 96%, at least 97%, at least 98%, or at least 99% identical to

(SEQ ID NO: 261)
ATCTATATCTGGGCCCCTCTGGCTGGCACATGCGGAGTTCTGCTG
CTCAGCCTGGTCATCACCCTGTACTGCAACCACAGA.

### Spacer Region

In some embodiments, a CAR of the present disclosure (e.g., the EMCN-specific, FLT3-specific, and/or CD33-specific CARs described herein) can also comprise a spacer region that links the extracellular antigen-binding domain to the transmembrane domain. The spacer region may be flexible enough to allow the antigen-binding domain to orient in different directions to facilitate antigen recognition. In some embodiments, the spacer region may be a hinge from a human protein. For example, the hinge may be a human Ig (immunoglobulin) hinge, including without limitation an IgG4 hinge, an IgG2 hinge, a CD8a hinge, or an IgD hinge. In some embodiments, the spacer region may comprise an IgG4 hinge, an IgG2 hinge, an IgD hinge, a CD28 hinge, a KIR2DS2 hinge, an LNGFR hinge, or a PDGFR-beta extracellular linker. In some embodiments, the spacer region is localized between the antigen-binding domain and the transmembrane domain. In some embodiments, a spacer region may comprise any of the amino acid sequences listed in Table 7, or an amino acid sequence that is at least 90%, at least 91%, at least 92%, at least 93%, at least 94%, at least 95%, at least 96%, at least 97%, at least 98%, or at least 99% identical to any of the amino acid sequences listed in Table 7. In some embodiments, nucleic acids encoding any of the spacer regions of the present disclosure may comprise any of the nucleic acid sequences listed in Table 8, or a nucleic acid sequence that is at least 90%, at least 91%, at least 92%, at least 93%, at least 94%, at least 95%, at least 96%, at least 97%, at least 98%, or at least 99% identical to any of the nucleic acid sequences listed in Table 8.

In some embodiments, an aCAR hinge domain includes a CD8 hinge. In some embodiments, an aCAR hinge domain includes a CD8 hinge having the amino acid sequence ALSNSIMYFSHFVPVFLPAKPTTTPAPRPPTPAPTIASQPLSLRPEACRPAAGGAVHTRGL DFACD (SEQ ID NO: 272). In some embodiments, an aCAR hinge domain includes a CD8 hinge encoded by a polynucleotide sequence having the sequence GCCCTGAGCAACAGCATCATGTACTTCAGCCACTTCGTGCCCGTGTTTCTGCCCGCC AAGCCTACAACAACCCCTGCTCCTAGACCACCTACACCAGCTCCTACAATCGCCAG CCAGCCTCTGTCTCTGAGGCCCGAAGCTTGTAGACCAGCTGCTGGCGGAGCCGTGC ATACAAGAGGACTGGATTTTGCCTGCGAC (SEQ ID NO: 284). In some embodiments, an aCAR hinge domain includes a CD8 hinge encoded by a polynucleotide sequence that is at least 90%, at least 91%, at least 92%, at least 93%, at least 94%, at least 95%, at least 96%, at least 97%, at least 98%, or at least 99% identical to

(SEQ ID NO: 284)
GCCCTGAGCAACAGCATCATGTACTTCAGCCACTTCGTGCCCGTG
TTTCTGCCCGCCAAGCCTACAACAACCCCTGCTCCTAGACCACCT
ACACCAGCTCCTACAATCGCCAGCCAGCCTCTGTCTCTGAGGCCC
GAAGCTTGTAGACCAGCTGCTGGCGGAGCCGTGCATACAAGAGGA
CTGGATTTTGCCTGCGAC.

In some embodiments, an iCAR hinge domain includes a CD8 hinge. In some embodiments, an iCAR hinge domain includes a CD8 hinge having the amino acid sequence TTTPAPRPPTPAPTIALQPLSLRPEACRPAAGGAVHTRGLDFACD (SEQ ID NO: 271). In some embodiments, an iCAR hinge domain includes a CD8 hinge encoded by a polynucleotide sequence having the sequence ACAACAACACCCGCACCTCGGCCTCCAACTCCAGCTCCAACAATTGCACTGCAACC CCTGAGTCTGAGGCCCGAGGCCTGTAGGCCAGCAGCTGGCGGAGCTGTTCACACTA GAGGCCTGGACTTTGCCTGTGAC (SEQ ID NO: 283). In some embodiments, an iCAR hinge domain includes a CD8 hinge encoded by a polynucleotide sequence that is at least 90%, at least 91%, at least 92%, at least 93%, at least 94%, at least 95%, at least 96%, at least 97%, at least 98%, or at least 99% identical to

(SEQ ID NO: 283)
ACAACAACACCCGCACCTCGGCCTCCAACTCCAGCTCCAACAATT
GCACTGCAACCCCTGAGTCTGAGGCCCGAGGCCTGTAGGCCAGCA
GCTGGCGGAGCTGTTCACACTAGAGGCCTGGACTTTGCCTGTGAC.

In some embodiments, an iCAR hinge domain includes a LIR1 hinge. In some embodiments, an iCAR hinge domain includes a LIR1 hinge having the amino acid sequence HPSDPLELVVSGPSGGPSSPTTGPTSTSGPEDQPLTPTGSDPQSGLGRHLGV (SEQ ID NO: 417). In some embodiments, an iCAR hinge domain includes a LIR1 hinge encoded by a polynucleotide sequence having the sequence CACCCATCCGATCCTCTCGAGCTGGTGGTTTCTGGACCTTCTGGCGGCCCTAGCAGC CCTACAACAGGACCTACAAGCACAAGCGGCCCTGAGGACCAACCTCTGACACCAAC AGGCAGCGATCCTCAGTCTGGACTGGGGAGACATCTGGGCGTT (SEQ ID NO: 418). In some embodiments, an iCAR hinge domain includes a LIR1 hinge encoded by a polynucleotide sequence that is at least 90%, at least 91%, at least 92%, at least 93%, at least 94%, at least 95%, at least 96%, at least 97%, at least 98%, or at least 99% identical to

(SEQ ID NO: 418)
CACCCATCCGATCCTCTCGAGCTGGTGGTTTCTGGACCTTCTGGCGGCCC
TAGCAGCCCTACAACAGGACCTACAAGCACAAGCGGCCCTGAGGACCAAC
CTCTGACACCAACAGGCAGCGATCCTCAGTCTGGACTGGGGAGACATCTG
GGCGTT.

TABLE 7
Spacer Amino Acid Sequences

| | SEQ | |
|---|---|---|
| | ID | |
| Amino Acid Sequence | NO: | Description |
| AAAIEVMYPPPYLDNEKSNGTIIH | 262 | CD28 hinge |
| VKGKHLCPSPLFPGPSKP | | |
| ESKYGPPCPSCP | 263 | IgG4 minimal hinge |
| ESKYGPPAPSAP | 264 | IgG4 minimal hinge, |
| | | no disulfides |
| ESKYGPPCPPCP | 265 | IgG4 S228P minimal |
| | | hinge, enhanced |
| | | disulfide formation |
| EPKSCDKTHTCP | 266 | IgG1 minimal hinge |
| AAAFVPVFLPAKPTTTPAPRPPTP | 267 | Extended CD8a hinge |
| APTIASQPLSLRPEACRPAAGGAV | | |
| HTRGLDFACDIYIWAPLAGTCGVL | | |
| LLSLVITLYCNHRN | | |
| ACPTGLYTHSGECCKACNLGEGVA | 268 | LNGFR hinge |
| QPCGANQTVCEPCLDSVTFSDVVS | | |
| ATEPCKPCTECVGLQSMSAPCVEA | | |
| DDAVCRCAYGYYQDETTGRCEACR | | |
| VCEAGSGLVFSCQDKQNTVCEECP | | |
| DGTYSDEADAEC | | |
| ACPTGLYTHSGECCKACNLGEGVA | 269 | Truncated LNGFR |
| QPCGANQTVC | | hinge (TNFR-Cys1) |
| AVGQDTQEVIVVPHSLPFKV | 270 | PDGFR-beta extra- |
| | | cellular linker |
| TTTPAPRPPTPAPTIALQPLSLRP | 271 | Example spacer |
| EACRPAAGGAVHTRGLDFACD | | derived from |
| | | CD8 hinge |
| ALSNSIMYFSHFVPVFLPAKPTTT | 272 | Example spacer |
| PAPRPPTPAPTIASQPLSLRPEAC | | derived from |
| RPAAGGAVHTRGLDFACD | | CD8 hinge |
| FVPVFLPAKPTTTPAPRPPTPAPT | 273 | Example spacer |
| IALQPLSLRPEACRPAAGGAVHTR | | |
| GLDFACD | | |
| HPSDPLELVVSGPSGGPSSPTTGP | 417 | LIR1 Hinge |
| TSTSGPEDQPLTPTGSDPQSGLGR | | |
| HLGV | | |

TABLE 8
Spacer Nucleic Acid Sequences

| Nucleic Acid Sequence | SEQ ID NO: | Description |
|---|---|---|
| GCAGCAGCTATCGAGGTGATGTATCCTCCGCCCTACC | 274 | CD28 hinge |
| TGGATAATGAAAAGAGTAATGGGACTATCATTCATGT | | |
| AAAAGGGAAGCATCTTTGTCCTTCTCCCCTTTTCCCC | | |
| GGTCCGTCTAAACCT | | |
| GAAAGCAAGTACGGTCCACCTTGCCCTAGCTGTCCG | 275 | IgG4 minimal hinge |
| GAATCCAAGTACGGCCCCCCAGCGCCTAGTGCCCCA | 276 | IgG4 minimal hinge, no |
| | | disulfides |
| GAATCTAAATATGGCCCGCCATGCCCGCCTTGCCCA | 277 | IgG4 S228P minimal hinge, |
| | | enhanced disulfide formation |
| GAACCGAAGTCTTGTGATAAAACTCATACGTGCCCG | 278 | IgG1 minimal hinge |
| GCTGCTGCTTTCGTACCCGTGTTCCTCCCTGCTAAGC | 279 | Extended CD8a hinge |
| CTACGACTACCCCCGCACCGAGACCACCCACGCCAGC | | |
| ACCCACGATTGCTAGCCAGCCCCTTAGTTTGCGACCA | | |
| GAAGCTTGTCGGCCTGCTGCTGGTGGCGCGGTACATA | | |
| CCCGCGGCCTTGATTTTGCTTGCGATATATATATCTG | | |
| GGCGCCTCTGGCCGGAACATGCGGGGTCCTCCTCCTT | | |
| TCTCTGGTTATTACTCTCTACTGTAATCACAGGAAT | | |
| GCCTGCCCGACCGGGCTCTACACTCATAGCGGGGAAT | 280 | LNGFR hinge |
| GTTGTAAGGCATGTAACTTGGGTGAGGGCGTCGCACA | | |
| GCCCTGCGGAGCTAACCAAACAGTGTGCGAACCCTGC | | |
| CTCGATAGTGTGACGTTCTCTGATGTTGTATCAGCTA | | |
| CAGAGCCTTGCAAACCATGTACTGAGTGCGTTGGACT | | |
| TCAGTCAATGAGCGCTCCATGTGTGGAGGCAGATGAT | | |
| GCGGTCTGTCGATGTGCTTACGGATACTACCAAGACG | | |
| AGACAACAGGGCGGTGCGAGGCCTGTAGAGTTTGTGA | | |
| GGCGGGCTCCGGGCTGGTGTTTTCATGTCAAGACAAG | | |
| CAAAATACGGTCTGTGAAGAGTGCCCTGATGGCACCT | | |
| ACTCAGACGAAGCAGATGCAGAATGC | | |
| GCCTGCCCTACAGGACTCTACACGCATAGCGGTGAGT | 281 | Truncated LNGFR hinge |
| GTTGTAAAGCATGCAACCTCGGGGAAGGTGTAGCCCA | | (TNFR-Cys1) |
| GCCATGCGGGGCTAACCAAACCGTTTGC | | |
| GCTGTGGGCCAGGACACGCAGGAGGTCATCGTGGTGC | 282 | PDGFR-beta extracellular |
| CACACTCCTTGCCCTTTAAGGTG | | linker |
| ACAACAACACCCGCACCTCGGCCTCCAACTCCAGCTC | 283 | Example spacer derived from |
| CAACAATTGCACTGCAACCCCTGAGTCTGAGGCCCGA | | CD8 hinge |
| GGCCTGTAGGCCAGCAGCTGGCGGAGCTGTTCACACT | | |
| AGAGGCCTGGACTTTGCCTGTGAC | | |
| GCCCTGAGCAACAGCATCATGTACTTCAGCCACTTCG | 284 | Example spacer derived from |
| TGCCCGTGTTTCTGCCCGCCAAGCCTACAACAACCCC | | CD8 hinge |
| TGCTCCTAGACCACCTACACCAGCTCCTACAATCGCC | | |
| AGCCAGCCTCTGTCTCTGAGGCCCGAAGCTTGTAGAC | | |
| CAGCTGCTGGCGGAGCCGTGCATACAAGAGGACTGGA | | |
| TTTTGCCTGCGAC | | |
| CACCCATCCGATCCTCTCGAGCTGGTGGTTTCTGGAC | 418 | LIR1 Hinge |
| CTTCTGGCGGCCCTAGCAGCCCTACAACAGGACCTAC | | |
| AAGCACAAGCGGCCCTGAGGACCAACCTCTGACACCA | | |
| ACAGGCAGCGATCCTCAGTCTGGACTGGGGAGACATC | | |
| TGGGCGTT | | |

In some embodiments, a CAR of the present disclosure may further include a short oligopeptide or polypeptide linker that is between 2 amino acid residues and 10 amino acid residues in length, and that may form the linkage between the transmembrane domain and the cytoplasmic region of the CAR. A non-limiting example of a suitable linker is a glycine-serine doublet. In some embodiments, the linker comprises the ammo acid sequence of GGCKJSGGCKJS (SEQ ID NO: 208).

In some aspects, the transmembrane domain further comprises at least a portion of an extracellular domain of the same protein.

### Intracellular Signaling Domains

In some embodiments, a CAR of the present disclosure (e.g., the EMCN-specific, FLT3-specific, and/or CD33-specific CARs described herein) comprises one or more cytoplasmic domains or regions. The cytoplasmic domain or region of the CAR may include an intracellular signaling domain.

Examples of suitable intracellular signaling domains that may be used in CARs of the present disclosure include, without limitation, cytoplasmic sequences of the T cell receptor (TCR) and co-receptors that act in concert to modulate signal transduction following antigen receptor engagement, as well as any derivative or variant of these sequences and any recombinant sequence that has the same functional capability.

Without wishing to be bound by theory, it is believed that signals generated through the TCR alone are insufficient for full activation of the T cell and that a secondary and/or co-stimulatory signal is thus also typically required for full activation. Accordingly, T cell activation may be mediated by two distinct classes of cytoplasmic signaling sequences, those that initiate antigen-dependent primary activation through the TCR (primary intracellular signaling domains) and those that act in an antigen-independent manner to provide a secondary or co-stimulatory signal (secondary cytoplasmic domain, e.g., a co-stimulatory domain). In addition, T cell signaling and function (e.g., an activating signaling cascade) can be negatively regulated by inhibitory receptors present in a T cell through intracellular inhibitory co-signaling domains.

In some embodiments, the intracellular signaling domain of a CAR of the present disclosure can include an inhibitory intracellular signaling domains. Examples of inhibitory intracellular domains that can be used include PD-1, CTLA4, TIGIT, BTLA, and LIR-1 (LILRB1), TIM3, KIR3DL1, NKG2A, LAG3, SLAP1, SLAP2, Dok-1, Dok-2, LAIR1, GRB-2, CD200R, SIRPα, HAVR, GITR, PD-L1, KIR2DL1, KIR2DL2, KIR2DL3, KIR3DL2, CD94, KLRG-1, CEACAM1, LIR2, LIR3, LIR5, SIGLEC-2, and SIGLEC-10. Table 10 and Table 11 provide the amino acid and nucleotide sequneces, respectively, of the exemplary inhibitory intracellular signaling domains. In some embodiments, the inhibitory intracellular signaling domain includes one or more intracellular inhibitory co-signaling domains (e.g., see Table 10 and Table 11. In some embodiments, the one or more intracellular inhibitory co-signaling domains are linked to other domains (e.g., a transmembrane domain) through a peptide linker (e.g., see Table 6) or a spacer or hinge sequence (e.g., see Tables 7 and 8). In some embodiments, when two or more intracellular inhibitory co-signaling domains are present, the two or more intracellular inhibitory co-signaling domains can be linked through a peptide linker (e.g., see Table 6) or a spacer or hinge sequence (e.g., see Tables 7 and 8). In some embodiments, the intracellular inhibitory co-signaling domain is an inhibitory domain. In some embodiments, the one or more intracellular inhibitory co-signaling domains of a chimeric protein comprises one or more ITIM-containing protein, or fragment(s) thereof. ITIMs are conserved amino acid sequences found in cytoplasmic tails of many inhibitory immune receptors. Examples of ITIM-containing proteins include, but are not limited to, PD-1, TIGIT, BTLA, and LIR-1 (LILRB1), TIM3, KIR3DL1, NKG2A, LAG3, LAIR1, SIRPα, KIR2DL1, KIR2DL2, KIR2DL3, KIR3DL2, CD94, KLRG-1, CEACAM1, LIR2, LIR3, LIR5, SIGLEC-2, and SIGLEC-10.

TABLE 10
Exemplary inhibitory intracellular signaling
domain amino acid sequences

| | SEQ | |
|---|---|---|
| Amino Acid Sequence | ID NO: | Description |
| CSRAARGTIGARRTGQPLKEDPSAVPVFSVDYGELDFQWREKTPEPPVP | 435 | PD-1 intracellular |
| CVPEQTEYATIVFPSGMGTSSPARRGSADGPRSAQPLRPEDGHCSWPL | | signaling domain |
| AVSLSKMLKKRSPLTTGVYVKMPPTEPECEKQFQPYFIPIN | 436 | CTLA4 |
| | | intracellular |
| | | signaling domain |
| RRHQGKQNELSDTAGREINLVDAHLKSEQTEASTRQNSQVLLSETGIYD | 437 | BTLA |
| NDPDLCFRMQEGSEVYSNPCLEENKPGIVYASLNHSVIGPNSRLARNVK | | intracellular |
| EAPTEYASICVRS | | signaling domain |
| LRHRRQGKHWTSTQRKADFQHPAGAVGPEPTDRGLQWRSSPAADAQEEN | 285 | LIR1 intracellular |
| LYAAVKHTQPEDGVEMDTRSPHDEDPQAVTYAEVKHSRPRREMASPPSP | | signaling domain |
| LSGEFLDTKDRQAEEDRQMDTEAAASEAPQDVTYAQLHSLTLRREATEP | | |
| PPSQEGPSPAVPSIYATLAIH | | |
| HLWCSNKKNAAVMDQEPAGNRTANSEDSDEQDPEEVTYAQLDHCVFTQR | 438 | KIR3DL1 |
| KITRPSQRPKTPPTDTILYTELPNAKPRSKVVSCP | | intracellular |
| | | signaling domain |
| AVSLSKMLKKRSPLTTGVGVKMPPTEPECEKQFQPYFIPIN | 439 | CTLA4 |
| | | intracellular |
| | | signaling domain |
| KEPASPLDKCHYTKDNGQFDQSAKQLNLEAYTIEQETALISNKNGKPKR | 440 | NKG2A |
| QQRKPNPPLNLDSYIVGQNDM | | (reversed) |
| | | intracellular |
| | | signaling domain |
| LTRKKKALRIHSVEGDLRRKSAGQEEWSPSAPSPPGSCVQAEAAPAGLC | 441 | TIGIT |
| GEQRGEDCAELHDYFNVLSYRSLGNCSFFTETG | | intracellular |
| | | signaling domain |
| MDNQGVIYSDLNLPPNPKRQQRKPKGNKNSILATEQEITYAELNLQKAS | 442 | NKG2A |
| QDFQGNDKTYHCKDLPSAPEK | | intracellular |
| | | signaling domain |
| MEAIAKYDFKATADDELSFKRGDILKVLNEECDQNWYKAELNGKDGFIP | 443 | GRB2 |
| KNYIEMKPHPWFFGKIPRAKAEEMLSKQRHDGAFLIRESESAPGDFSLS | | intracellular |
| VKFGNDVQHFKVLRDGAGKYFLWVVKFNSLNELVDYHRSTSVSRNQQIF | | signaling domain |
| LRDIEQVPQQPTYVQALFDFDPQEDGELGFRRGDFIHVMDNSDPNWWKG | | |
| ACHGQTGMFPRNYVTPVNRNV | | |
| MDGAVMEGPLFLQSQRFGTKRWRKTWAVLYPASPHGVARLEFFDHKGSS | 444 | Dok-1 |
| SGGGRGSSRRLDCKVIRLAECVSVAPVTVETPPEPGATAFRLDTAQRSH | | intracellular |
| LLAADAPSSAAWVQTLCRNAFPKGSWTLAPTDNPPKLSALEMLENSLYS | | signaling domain |
| PTWEGSQFWVTVQRTEAAERCGLHGSYVLRVEAERLTLLTVGAQSQILE | | |
| PLLSWPYTLLRRYGRDKVMFSFEAGRRCPSGPGTFTFQTAQGNDIFQAV | | |
| ETAIHRQKAQGKAGQGHDVLRADSHEGEVAEGKLPSPPGPQELLDSPPA | | |
| LYAEPLDSLRIAPCPSQDSLYSDPLDSTSAQAGEGVQRKKPLYWDLYEH | | |
| AQQQLLKAKLTDPKEDPIYDEPEGLAPVPPQGLYDLPREPKDAWWCQAR | | |
| VKEEGYELPYNPATDDYAVPPPRSTKPLLAPKPQGPAFPEPGTATGSGI | | |
| KSHNSALYSQVQKSGASGSWDCGLSRVGTDKTGVKSEGST | | |
| MGDGAVKQGFLYLQQQQTFGKKWRRFGASLYGGSDCALARLELQEGPEK | 445 | Dok-2 |
| PRRCEAARKVIRLSDCLRVAEAGGEASSPRDTSAFFLETKERLYLLAAP | | intracellular |
| AAERGDWVQAICLLAFPGQRKELSGPEGKQSRPCMEENELYSSAVTVGP | | signaling domain |
| HKEFAVTMRPTEASERCHLRGSYTLRAGESALELWGGPEPGTQLYDWPY | | |
| RFLRRFGRDKVTFSFEAGRRCVSGEGNFEFETRQGNEIFLALEEAISAQ | | |
| KNAAPATPQPQPATIPASLPRPDSPYSRPHDSLPPPSPTTPVPAPRPRG | | |
| QEGEYAVPFDAVARSLGKNFRGILAVPPQLLADPLYDSIEETLPPRPDH | | |
| IYDEPEGVAALSLYDSPQEPRGEAWRRQATADRDPAGLQHVQPAGQDFS | | |
| ASGWQPGTEYDNVVLKKGPK | | |
| PAPAERPLPNPEGLDSDFLAVLSDYPSPDISPPIFRRGEKLRVISDEGG | 446 | SLAP1ₐₐ₈₋ₐₐ₂₇₆ |
| WWKAISLSTGRESYIPGICVARVYHGWLFEGLGRDKAEELLQLPDTKVG | | intracellular |
| SFMIRESETKKGFYSLSVRHRQVKHYRIFRLPNNWYYISPRLTFQCLED | | signaling domain |
| LVNHYSEVADGLCCVLTTPCLTQSTAAPAVRASSSPVTLRQKTVDWRRV | | |
| SRLQEDPEGTENPLGVDESLFSYGLRESIASYLSLTSEDNTSFDRKKKS | | |
| ISLMYGGSKRKSSFFSSPPYFED | | |
| PAPAERPLPNPEGLDSDFLAVLSDYPSPDISPPIFRRGEKLRVISDEGG | 447 | SLAP1ₐₐ₈₋ₐₐ₂₄₇ |
| WWKAISLSTGRESYIPGICVARVYHGWLFEGLGRDKAEELLQLPDTKVG | | intracellular |
| SFMIRESETKKGFYSLSVRHRQVKHYRIFRLPNNWYYISPRLTFQCLED | | signaling domain |
| LVNHYSEVADGLCCVLTTPCLTQSTAAPAVRASSSPVTLRQKTVDWRRV | | |
| SRLQEDPEGTENPLGVDESLFSYGLRESIASYLSLTSEDNTSF | | |
| RKSLPSPSLSSSVQGQGPVTMEAERSKATAVALGSFPAGGPAELSLRLG | 448 | SLAP2ₐₐ₈₋ₐₐ₂₆₁ |
| EPLTIVSEDGDWWTVLSEVSGREYNIPSVHVAKVSHGWLYEGLSREKAE | | intracellular |
| ELLLLPGNPGGAFLIRESQTRRGSYSLSVRLSRPASWDRIRHYRIHCLD | | signaling domain |
| NGWLYISPRLTFPSLQALVDHYSELADDICCLLKEPCVLQRAGPLPGKD | | |
| IPLPVTVQRTPLNWKELDSSLLFSEAATGEESLLSEGLRESLSFYISLN | | |
| DEAVSLDDA | | |
| KVNGCRKYKLNKTESTPVVEEDEMQPYASYTEKNNPLYDTTNKVKASEA | 449 | CD200R |
| LQSEVDTDLHTL | | intracellular |
| | | signaling domain |
| RIRQKKAQGSTSSTRLHEPEKNAREITQDTNDITYADLNLPKGKKPAPQ | 450 | SIRPα |
| AAEPNNHTEYASIQTSPQPASEDTLTYADLDMVHLNRTPKQPAPKPEPS | | intracellular |
| FSEYASVQVPRK | | signaling domain |
| HRWCSNKKNAAVMDQESAGNRTANSEDSDEQDPQEVTYTQLNHCVFTQR | 451 | KIR2DL1 |
| KITRPSQRPKTPPTDIIVYTELPNAESRSKVVSCP | | intracellular |
| | | signaling domain |
| MTDSVIYSMLELPTATQAQNDYGPQQKSSSSRPSCSCLGSG | 452 | KLRG1 |
| | | intracellular |
| | | signaling domain |
| HRQNQIKQGPPRSKDEEQKPQQRPDLAVDVLERTADKATVNGLPEKDRE | 453 | LAIR1 |
| TDTSALAAGSSQEVTYAQLDHWALTQRTARAVSPQSTKPMAESITYAAV | | intracellular |
| ARH | | signaling domain |
| LRHRRQGKHWTSTQRKADFQHPAGAVGPEPTDRGLQWRSSPAADAQEEN | 454 | LIR2 intracellular |
| LYAAVKDTQPEDGVEMDTRAAASEAPQDVTYAQLHSLTLRRKATEPPPS | | signaling domain |
| QEREPPAEPSIYATLAIH | | |
| RRQRHSKHRTSDQRKTDFQRPAGAAETEPKDRGLLRRSSPAADVQEENL | 455 | LIR3 intracellular |
| YAAVKDTQSEDRVELDSQSPHDEDPQAVTYAPVKHSSPRREMASPPSSL | | signaling domain |
| SGEFLDTKDRQVEEDRQMDTEAAASEASQDVTYAQLHSLTLRRKATEPP | | |
| PSQEGEPPAEPSIYATLAIH | | |
| QHWRQGKHRTLAQRQADFQRPPGAAEPEPKDGGLQRRSSPAADVQGENF | 456 | LIR5 intracellular |
| CAAVKNTQPEDGVEMDTRQSPHDEDPQAVTYAKVKHSRPRREMASPPSP | | signaling domain |
| LSGEFLDTKDRQAEEDRQMDTEAAASEAPQDVTYAQLHSFTLRQKATEP | | |
| PPSQEGASPAEPSVYATLAIH | | |
| KLQRRWKRTQSQQGLQENSSGQSFFVRNKKVRRAPLSEGPHSLGCYNPM | 457 | SIGLEC-2 |
| MEDGISYTTLRFPEMNIPRTGDAESSEMQRPPPDCDDTVTYSALHKRQV | | intracellular |
| GDYENVIPDFPEDEGIHYSELIQFGVGERPQAQENVDYVILKH | | signaling domain |
| KILPKRRTQTETPRPRFSRHSTILDYINVVPTAGPLAQKRNQKATPNSP | 458 | SIGLEC-10 |
| RTPLPPGAPSPESKKNQKKQYQLPSFPEPKSSTQAPESQESQEELHYAT | | intracellular |
| LNFPGVRPRPEARMPKGTQADYAEVKFQ | | signaling domain |

TABLE 11
Exemplary inhibitory intracellular signaling domain
nucleic acid sequences

| | SEQ | Description |
|---|---|---|
| Nucleic Acid Sequence | ID NO: | |
| TGTAGCAGAGCCGCCAGAGGAACAATCGGCGCCAGAAGAACAGGCC | 459 | PD-1 intracellular |
| AGCCTCTGAAAGAGGACCCCTCTGCCGTTCCTGTGTTCAGCGTGGA | | signaling domain |
| CTATGGCGAGCTGGATTTCCAGTGGCGGGAAAAGACACCCGAGCCT | | |
| CCAGTGCCTTGTGTGCCTGAGCAGACAGAGTACGCCACCATCGTGT | | |
| TCCCTAGCGGCATGGGCACATCTAGCCCTGCCAGAAGAGGATCTGC | | |
| CGACGGACCTAGATCTGCCCAGCCTCTTAGACCTGAGGACGGCCAC | | |
| TGTTCTTGGCCTCTT | | |
| TGTAGCCGAGCGGCCAGAGGCACAATCGGGGCAAGACGAACAGGAC | 460 | PD-1 intracellular |
| AGCCGCTCAAAGAGGACCCCAGTGCGGTCCCCGTTTTCTCCGTGGA | | signaling domain |
| TTACGGAGAACTGGATTTCCAGTGGCGGGAGAAGACACCAGAGCCC | | |
| CCGGTGCCCTGCGTGCCGGAGCAGACTGAGTACGCCACGATTGTGT | | |
| TTCCCTCTGGAATGGGGACTTCATCCCCCGCTAGGCGCGGCTCAGC | | |
| TGATGGCCCAAGATCCGCTCAACCGTTGCGGCCAGAGGACGGGCAT | | |
| TGCAGTTGGCCTCTG | | |
| GCCGTGTCTCTGAGCAAGATGCTGAAGAAGCGGAGCCCTCTGACCA | 461 | CTLA4 |
| CCGGCGTGTACGTGAAAATGCCTCCTACCGAGCCTGAGTGCGAGAA | | intracellular |
| GCAGTTCCAGCCTTACTTCATCCCCATCAAC | | signaling domain |
| AGGAGACATCAGGGGAAGCAGAATGAACTCAGCGATACAGCAGGGC | 462 | BTLA |
| GAGAAATTAATTTGGTAGACGCGCATCTGAAGTCCGAACAGACAGA | | intracellular |
| GGCTTCTACTAGACAGAACTCCCAAGTTTTGTTGAGTGAGACGGGG | | signaling domain |
| ATCTATGATAATGATCCCGATCTGTGTTTTAGAATGCAGGAGGGTA | | |
| GTGAAGTCTACTCAAACCCGTGCCTGGAAGAAAATAAGCCCGGCAT | | |
| TGTTTACGCTAGTTTGAATCATTCTGTAATAGGCCCGAACTCCAGA | | |
| CTGGCTCGCAATGTGAAGGAGGCCCCAACTGAGTATGCGTCCATTT | | |
| GCGTGCGGTCT | | |
| AGAAGACATCAGGGGAAGCAGAATGAACTCAGCGATACAGCAGGGC | 463 | BTLA |
| GAGAAATTAATTTGGTAGACGCGCATCTGAAGTCCGAACAGACAGA | | intracellular |
| GGCTTCTACTAGACAGAACTCCCAAGTTTTGTTGAGTGAGACGGGG | | signaling domain |
| ATCTATGATAATGATCCCGATCTGTGTTTTAGAATGCAGGAGGGTA | | |
| GTGAAGTCTACTCAAACCCGTGCCTGGAAGAAAATAAGCCCGGCAT | | |
| TGTTTACGCTAGTTTGAATCATTCTGTAATAGGCCCGAACTCCAGA | | |
| CTGGCTCGCAATGTGAAGGAGGCCCCAACTGAGTATGCGTCCATTT | | |
| GCGTGCGGTCT | | |
| AGAAGGCACCAGGGAAAGCAGAACGAGCTGAGCGATACCGCCGGCA | 464 | BTLA |
| GAGAAATCAACCTGGTGGACGCCCACCTGAAAAGCGAGCAGACAGA | | intracellular |
| GGCCAGCACCAGACAGAATAGCCAGGTGCTGCTGAGCGAGACAGGC | | signaling domain |
| ATCTACGACAACGACCCCGACCTGTGCTTCCGGATGCAAGAGGGAA | | |
| GCGAGGTGTACAGCAACCCCTGCCTGGAAGAGAACAAGCCCGGCAT | | |
| CGTGTACGCTAGCCTGAACCACTCTGTGATCGGCCCCAATTCCAGA | | |
| CTGGCCCGGAACGTGAAAGAGGCCCCTACAGAGTACGCCAGCATCT | | |
| GCGTCAGAAGC | | |
| TTGCGCCACAGACGGCAGGGAAAGCACTGGACTAGTACGCAGAGGA | 465 | LIR1 intracellular |
| AAGCGGACTTCCAGCATCCCGCAGGAGCCGTGGGGCCTGAACCCAC | | signaling domain |
| TGATCGCGGCCTTCAATGGAGGTCTAGCCCGGCGGCAGACGCACAA | | |
| GAGGAAAACTTGTACGCAGCCGTTAAGCACACCCAACCGGAGGACG | | |
| GCGTTGAGATGGATACCCGCTCCCCTCACGATGAAGACCCTCAAGC | | |
| AGTCACTTACGCGGAAGTAAAGCATAGCCGCCCCAGACGGGAAATG | | |
| GCTAGCCCGCCGTCCCCCCTTAGCGGGGAATTTCTGGACACTAAAG | | |
| ATAGGCAGGCGGAAGAGGACCGCCAAATGGATACAGAGGCGGCGGC | | |
| AAGTGAAGCACCTCAAGACGTTACTTACGCTCAACTTCACAGCCTT | | |
| ACCCTCAGGCGAGAAGCGACTGAACCACCCCCTTCCCAAGAAGGGC | | |
| CAAGCCCAGCGGTTCCTTCTATCTATGCTACTCTTGCTATTCAC | | |
| CTGCGGCACAGACGGCAGGGCAAGCACTGGACAAGCACACAGAGAA | 466 | LIR1 intracellular |
| AGGCCGACTTTCAGCACCCTGCTGGTGCCGTTGGACCTGAGCCTAC | | signaling domain |
| AGATAGAGGACTGCAGTGGCGGTCTAGCCCTGCCGCTGATGCTCAA | | |
| GAGGAAAACCTGTACGCCGCCGTGAAGCACACCCAACCTGAAGATG | | |
| GCGTGGAAATGGACACCAGATCTCCCCACGATGAGGACCCTCAGGC | | |
| CGTGACATATGCCGAAGTGAAGCACTCCCGGCCTCGGAGAGAAATG | | |
| GCTAGCCCTCCAAGTCCTCTGAGCGGCGAGTTCCTGGACACCAAGG | | |
| ATAGACAGGCCGAAGAGGACCGGCAGATGGATACAGAAGCTGCCGC | | |
| ATCTGAGGCCCCACAGGATGTGACTTATGCCCAGCTGCACAGCCTG | | |
| ACACTGCGGAGAGAAGCCACAGAGCCTCCACCTTCTCAAGAGGGCC | | |
| CATCTCCAGCCGTGCCTAGCATCTATGCCACACTGGCCATCCAC | | |
| GCCGTGTCACTTAGTAAGATGCTGAAGAAGAGGTCACCACTGACGA | 467 | CTLA4 |
| CAGGGGTTGGAGTGAAGATGCCACCCACAGAACCCGAATGTGAGAA | | intracellular |
| GCAATTCCAGCCTTATTTCATTCCAATAAAT | | signaling domain |
| CATCTGTGGTGTTCTAATAAGAAGAATGCTGCTGTGATGGATCAAG | 468 | KIR3DL1 |
| AGCCCGCTGGTAACAGAACGGCCAACAGTGAAGATAGCGATGAGCA | | intracellular |
| GGACCCAGAAGAAGTGACCTACGCCCAACTCGACCACTGTGTTTTT | | signaling domain |
| ACGCAGCGGAAAATCACTCGACCCTCTCAACGACCCAAAACGCCGC | | |
| CTACGGACACCATACTCTACACCGAACTGCCGAACGCCAAACCACG | | |
| GTCCAAGGTGGTATCATGTCCG | | |
| CTGCGGCACAGAAGGCAGGGCAAGCACTGGACAAGCACCCAGAGAA | 469 | LIR1 intracellular |
| AGGCCGATTTTCAGCACCCTGCTGGCGCCGTTGGACCTGAGCCTAC | | signaling domain |
| AGATAGAGGACTGCAGTGGCGGTCTAGCCCTGCTGCCGATGCTCAA | | |
| GAGGAAAACCTGTACGCCGCCGTGAAGCACACCCAACCTGAAGATG | | |
| GCGTGGAAATGGACACCAGATCTCCCCACGATGAGGACCCTCAGGC | | |
| CGTGACATACGCTGAAGTGAAGCACTCCCGGCCTCGGAGAGAAATG | | |
| GCTAGCCCTCCAAGTCCTCTGAGCGGCGAGTTCCTGGACACCAAGG | | |
| ATAGACAGGCCGAAGAGGACCGGCAGATGGATACAGAAGCTGCCGC | | |
| CTCTGAAGCCCCACAGGATGTGACATATGCCCAGCTGCATAGCCTG | | |
| ACACTGCGGAGAGAAGCCACAGAGCCTCCACCTTCTCAAGAGGGCC | | |
| CATCTCCAGCCGTGCCTAGCATCTATGCCACACTGGCCATTCAC | | |
| AAGGAGCCTGCGTCCCCGTTGGATAAATGCCACTATACTAAGGATA | 470 | NKG2A |
| ACGGTCAGTTCGATCAGAGTGCAAAGCAACTTAACTTGGAGGCTTA | | (reversed) |
| CACTATAGAGCAAGAAACAGCGCTGATAAGTAATAAGAACGGTAAG | | intracellular |
| CCAAAGCGACAGCAGAGGAAACCCAATCCTCCGCTTAACTTGGATA | | signaling domain |
| GCTACATCGTCGGGCAAAATGACATG | | |
| CTGACCAGAAAGAAGAAGGCCCTGAGAATCCACAGCGTGGAAGGCG | 471 | TIGIT |
| ACCTGCGGAGAAAGTCTGCCGGACAAGAAGAGTGGTCCCCTAGCGC | | intracellular |
| TCCATCTCCACCTGGATCTTGTGTGCAGGCCGAAGCAGCTCCTGCT | | signaling domain |
| GGACTGTGTGGCGAACAGAGAGGCGAAGATTGCGCCGAGCTGCACG | | |
| ACTACTTCAACGTGCTGAGCTACAGAAGCCTGGGCAACTGCAGCTT | | |
| CTTCACCGAGACAGGA | | |
| ATGGACAACCAGGGCGTGATCTACAGCGACCTGAACCTGCCTCCTA | 472 | NKG2A |
| ATCCTAAGCGGCAGCAGAGAAAGCCCAAGGGCAACAAGAACAGCAT | | intracellular |
| CCTGGCCACCGAGCAAGAGATCACCTACGCCGAGCTGAATCTGCAG | | signaling domain |
| AAGGCCAGCCAGGACTTCCAGGGCAACGACAAGACCTACCACTGCA | | |
| AGGACCTGCCTAGCGCTCCCGAGAAG | | |
| ATGGACAACCAGGGCGTCATCTACAGCGACCTGAACCTGCCTCCTA | 473 | NKG2A |
| ATCCAAAGCGGCAGCAGCGGAAGCCCAAGGGCAACAAGAATAGCAT | | intracellular |
| CCTGGCCACCGAGCAAGAGATCACCTACGCCGAGCTGAATCTGCAG | | signaling domain |
| AAGGCCAGCCAGGATTTCCAGGGCAACGACAAGACCTACCACTGCA | | (codon optimized) |
| AGGACCTGCCTAGCGCTCCTGAGAAA | | |
| ATGGAAGCCATTGCCAAATACGACTTCAAGGCCACCGCCGACGACG | 474 | GRB2 |
| AGCTGAGCTTCAAGAGAGGCGACATCCTGAAGGTGCTGAACGAGGA | | intracellular |
| ATGCGACCAGAACTGGTACAAGGCCGAGCTGAACGGCAAGGACGGC | | signaling domain |
| TTCATCCCCAAGAACTACATCGAGATGAAGCCCCATCCATGGTTCT | | |
| TCGGCAAGATCCCCAGAGCCAAGGCCGAAGAGATGCTGAGCAAGCA | | |
| GAGACACGACGGCGCCTTTCTGATCCGGGAATCTGAATCTGCCCCT | | |
| GGCGACTTCAGCCTGAGCGTGAAGTTCGGCAACGACGTGCAGCACT | | |
| TCAAGGTCCTGAGAGATGGCGCCGGAAAGTACTTCCTGTGGGTCGT | | |
| GAAGTTTAACAGCCTGAACGAGCTGGTGGACTACCACAGATCCACC | | |
| AGCGTGTCCCGGAACCAGCAGATCTTCCTGCGGGACATCGAACAGG | | |
| TGCCACAGCAGCCAACATACGTGCAGGCCCTGTTCGACTTCGACCC | | |
| TCAAGAGGATGGCGAGCTGGGCTTTAGACGGGGCGATTTCATCCAC | | |
| GTGATGGACAACAGCGACCCCAACTGGTGGAAGGGCGCTTGTCATG | | |
| GACAGACCGGCATGTTCCCCAGAAACTACGTGACCCCTGTGAACCG | | |
| GAACGTG | | |
| ATGGATGGCGCCGTGATGGAAGGCCCTCTGTTTCTCCAGAGCCAGA | 475 | Dok-1 |
| GATTCGGCACCAAGCGGTGGCGGAAAACATGGGCCGTTCTGTACCC | | intracellular |
| TGCCTCTCCTCATGGCGTGGCCCGGCTGGAATTTTTCGATCACAAG | | signaling domain |
| GGCTCTAGCAGCGGCGGAGGCAGAGGATCTAGTAGACGGCTGGACT | | |
| GCAAAGTGATCCGGCTGGCCGAGTGTGTGTCTGTGGCTCCTGTGAC | | |
| CGTGGAAACCCCTCCTGAACCTGGCGCCACAGCCTTCAGACTGGAT | | |
| ACAGCCCAGAGAAGCCATCTGCTGGCCGCCGATGCTCCTTCTTCTG | | |
| CTGCTTGGGTGCAGACCCTGTGCCGGAACGCTTTTCCTAAAGGCAG | | |
| CTGGACACTGGCCCCTACCGACAATCCTCCTAAGCTGAGCGCCCTG | | |
| GAAATGCTGGAAAACAGCCTGTACAGCCCCACCTGGGAGGGCTCTC | | |
| AGTTTTGGGTCACCGTGCAGAGAACAGAGGCCGCCGAAAGATGTGG | | |
| CCTGCACGGCTCTTATGTGCTGAGAGTGGAAGCCGAGAGACTGACC | | |
| CTGCTGACAGTGGGAGCCCAGTCTCAGATCCTGGAACCTCTGCTGA | | |
| GCTGGCCCTACACACTGCTGAGAAGATACGGCCGGGACAAAGTGAT | | |
| GTTCAGCTTCGAGGCCGGCAGAAGATGTCCTTCTGGCCCTGGCACA | | |
| TTCACATTTCAGACAGCCCAGGGCAACGACATCTTCCAGGCTGTGG | | |
| AAACCGCCATCCACAGACAGAAGGCCCAGGGAAAAGCCGGCCAGGG | | |
| ACACGATGTTCTGAGAGCCGATTCTCACGAGGGCGAAGTGGCCGAG | | |
| GGAAAGCTTCCTTCTCCACCTGGACCTCAAGAGCTGCTGGATAGCC | | |
| CTCCTGCTCTGTATGCCGAGCCTCTGGACAGCCTGAGAATCGCCCC | | |
| TTGTCCAAGCCAGGACTCTCTGTACAGCGATCCCCTGGATAGCACA | | |
| TCTGCCCAAGCTGGCGAAGGCGTGCAGAGGAAGAAGCCCCTGTACT | | |
| GGGATCTGTACGAGCACGCTCAGCAGCAACTGCTGAAGGCCAAGCT | | |
| GACAGACCCCAAAGAGGACCCCATCTACGACGAGCCTGAAGGACTT | | |
| GCTCCAGTGCCACCTCAGGGCCTGTACGATCTGCCTAGAGAGCCTA | | |
| AAGACGCCTGGTGGTGTCAGGCCAGAGTGAAAGAGGAAGGCTACGA | | |
| GCTGCCTTACAACCCCGCCACCGATGATTATGCCGTGCCTCCTCCA | | |
| AGAAGCACCAAACCACTGCTGGCCCCAAAGCCTCAGGGACCTGCTT | | |
| TTCCTGAGCCTGGAACAGCCACAGGCAGCGGCATCAAGAGCCACAA | | |
| TAGCGCCCTGTATAGCCAGGTGCAGAAAAGCGGCGCCAGCGGCTCT | | |
| TGGGATTGTGGACTTAGCAGAGTGGGCACCGACAAGACCGGCGTGA | | |
| AGTCTGAGGGAAGCACA | | |
| ATGGGAGATGGCGCCGTGAAGCAGGGCTTTCTGTATCTCCAGCAGC | 476 | Dok-2 |
| AGCAGACCTTCGGCAAGAAGTGGCGGAGATTTGGCGCCTCTCTGTA | | intracellular |
| CGGCGGCTCTGATTGTGCTCTGGCCCGACTGGAACTGCAAGAGGGA | | signaling domain |
| CCTGAGAAGCCCAGAAGATGCGAGGCCGCCAGAAAAGTGATCCGGC | | |
| TGAGCGATTGTCTGAGAGTGGCTGAAGCAGGCGGCGAAGCCAGCTC | | |
| TCCTAGAGATACCAGCGCATTCTTCCTGGAAACAAAAGAGCGGCTG | | |
| TACCTGCTGGCCGCTCCTGCTGCTGAAAGAGGCGATTGGGTCCAAG | | |
| CCATCTGCCTGCTGGCTTTTCCCGGCCAGAGAAAAGAGCTGTCTGG | | |
| CCCTGAGGGCAAGCAGAGCAGGCCTTGCATGGAAGAGAACGAGCTG | | |
| TACTCCAGCGCCGTGACAGTGGGCCCTCACAAAGAATTTGCCGTGA | | |
| CCATGAGGCCCACCGAGGCCAGCGAAAGATGTCACCTGAGAGGCAG | | |
| CTACACCCTGAGAGCCGGCGAATCTGCTCTGGAACTTTGGGGAGGA | | |
| CCTGAGCCTGGCACACAGCTGTACGACTGGCCCTACAGATTCCTGC | | |
| GGAGATTCGGCCGGGACAAAGTGACCTTCAGCTTTGAGGCTGGCCG | | |
| CAGATGTGTGTCCGGCGAGGGCAATTTCGAGTTCGAGACAAGACAG | | |
| GGCAACGAGATCTTCCTGGCTCTGGAAGAGGCCATCAGCGCCCAGA | | |
| AAAATGCTGCCCCTGCTACACCTCAGCCTCAGCCTGCTACAATCCC | | |
| TGCCTCTCTGCCCAGACCTGACAGCCCTTATAGCAGACCCCACGAC | | |
| TCTCTGCCTCCACCTTCTCCAACAACACCCGTGCCTGCTCCTAGAC | | |
| CTAGAGGACAAGAGGGCGAGTACGCCGTGCCTTTTGATGCCGTGGC | | |
| TAGAAGCCTGGGCAAGAACTTCAGAGGCATCCTGGCTGTGCCTCCA | | |
| CAGCTGCTGGCTGACCCTCTGTACGACAGCATCGAGGAAACCCTGC | | |
| CTCCAAGACCTGACCACATCTACGACGAGCCTGAAGGCGTGGCAGC | | |
| CCTGTCTCTGTATGACTCCCCTCAAGAGCCTAGAGGCGAAGCCTGG | | |
| CGTAGACAGGCTACCGCCGATAGAGATCCTGCCGGACTGCAACATG | | |
| TGCAGCCAGCCGGCCAGGATTTTTCTGCCTCTGGATGGCAGCCAGG | | |
| CACCGAGTACGATAACGTGGTGCTGAAGAAGGGCCCCAAG | | |
| CCAGCCCCAGCGGAGCGACCGCTGCCAAACCCTGAAGGGCTCGACA | 477 | SLAP1ₐₐ₈₋ₐₐ₂₇₆ |
| GTGACTTTTTGGCTGTCCTCTCCGACTATCCTAGTCCCGATATCAG | | intracellular |
| CCCCCCGATATTCAGGCGCGGTGAAAAACTCCGAGTCATCAGCGAT | | signaling domain |
| GAGGGGGGTTGGTGGAAAGCCATCAGCCTGAGTACCGGACGAGAGT | | |
| CATACATTCCTGGAATATGTGTAGCGAGGGTGTACCACGGTTGGCT | | |
| GTTCGAGGGTCTGGGAAGAGATAAGGCCGAGGAACTTCTCCAACTC | | |
| CCAGATACAAAAGTCGGTAGTTTTATGATTCGGGAAAGTGAAACTA | | |
| AAAAGGGGTTCTATAGCCTCTCAGTTCGGCATAGGCAGGTCAAGCA | | |
| TTATCGGATATTTCGCTTGCCTAACAACTGGTACTACATAAGTCCC | | |
| CGACTCACATTCCAATGCCTGGAGGACCTCGTGAATCACTATTCAG | | |
| AAGTTGCAGATGGCCTCTGCTGCGTACTCACGACACCCTGCCTTAC | | |
| CCAGAGTACAGCGGCCCCGGCTGTTCGGGCATCTTCCAGCCCAGTA | | |
| ACACTCAGGCAAAAAACTGTGGATTGGCGCAGAGTCTCACGCCTTC | | |
| AGGAAGATCCTGAGGGTACGGAAAATCCGCTCGGTGTGGACGAGTC | | |
| ACTGTTCTCCTATGGCTTGAGGGAATCTATAGCGTCTTACCTTTCC | | |
| TTGACGTCTGAAGATAATACGTCTTTCGATCGCAAAAAAAAATCAA | | |
| TATCTCTTATGTATGGCGGAAGCAAAAGGAAATCATCATTCTTTTC | | |
| CTCTCCACCATATTTCGAAGAT | | |
| CCAGCCCCAGCGGAGCGACCGCTGCCAAACCCTGAAGGGCTCGACA | 478 | SLAP1ₐₐ₈₋ₐₐ₂₄₇ |
| GTGACTTTTTGGCTGTCCTCTCCGACTATCCTAGTCCCGATATCAG | | intracellular |
| CCCCCCGATATTCAGGCGCGGTGAAAAACTCCGAGTCATCAGCGAT | | signaling domain |
| GAGGGGGGTTGGTGGAAAGCCATCAGCCTGAGTACCGGACGAGAGT | | |
| CATACATTCCTGGAATATGTGTAGCGAGGGTGTACCACGGTTGGCT | | |
| GTTCGAGGGTCTGGGAAGAGATAAGGCCGAGGAACTTCTCCAACTC | | |
| CCAGATACAAAAGTCGGTAGTTTTATGATTCGGGAAAGTGAAACTA | | |
| AAAAGGGGTTCTATAGCCTCTCAGTTCGGCATAGGCAGGTCAAGCA | | |
| TTATCGGATATTTCGCTTGCCTAACAACTGGTACTACATAAGTCCC | | |
| CGACTCACATTCCAATGCCTGGAGGACCTCGTGAATCACTATTCAG | | |
| AAGTTGCAGATGGCCTCTGCTGCGTACTCACGACACCCTGCCTTAC | | |
| CCAGAGTACAGCGGCCCCGGCTGTTCGGGCATCTTCCAGCCCAGTA | | |
| ACACTCAGGCAAAAAACTGTGGATTGGCGCAGAGTCTCACGCCTTC | | |
| AGGAAGATCCTGAGGGTACGGAAAATCCGCTCGGTGTGGACGAGTC | | |
| ACTGTTCTCCTATGGCTTGAGGGAATCTATAGCGTCTTACCTTTCC | | |
| TTGACGTCTGAAGATAATACGTCTTTC | | |
| AGAAAATCCCTCCCCAGTCCAAGCCTGTCTAGTAGCGTTCAGGGTC | 479 | SLAP2ₐₐ₈₋ₐ₂₆₁ |
| AGGGCCCAGTTACTATGGAAGCGGAACGATCCAAGGCTACCGCAGT | | intracellular |
| TGCTCTGGGTTCATTCCCGGCTGGAGGGCCAGCGGAACTCTCCTTG | | signaling domain |
| CGCCTGGGTGAGCCACTTACCATCGTTTCTGAGGACGGAGATTGGT | | |
| GGACGGTTCTTTCTGAAGTATCTGGGAGAGAATACAACATACCGTC | | |
| AGTTCATGTCGCAAAAGTTTCACACGGTTGGCTTTACGAGGGACTC | | |
| AGCAGGGAAAAAGCGGAGGAATTGTTGCTTTTGCCCGGCAATCCTG | | |
| GCGGAGCATTTTTGATAAGAGAGAGTCAGACCCGGAGAGGCAGTTA | | |
| TTCCCTGTCAGTTAGACTCAGCAGACCTGCGAGTTGGGATAGGATT | | |
| CGGCACTACAGGATTCACTGCCTTGATAACGGCTGGCTGTATATAT | | |
| CTCCTCGCCTGACATTCCCTAGCCTCCAAGCGTTGGTTGATCATTA | | |
| CTCTGAACTGGCAGACGATATCTGCTGCCTGCTCAAGGAACCGTGC | | |
| GTCCTCCAGAGGGCAGGCCCACTTCCTGGCAAGGATATTCCACTTC | | |
| CAGTCACGGTTCAAAGAACCCCCCTCAATTGGAAGGAGCTGGATAG | | |
| CTCTCTCCTCTTTTCCGAGGCCGCTACAGGTGAAGAATCTCTGTTG | | |
| TCTGAAGGATTGAGAGAGAGTCTCTCCTTCTACATCTCTCTGAATG | | |
| ATGAAGCAGTGTCATTGGACGACGCA | | |
| AAAGTGAACGGCTGCCGGAAGTACAAGCTGAACAAGACCGAGAGCA | 480 | CD200R |
| CCCCTGTGGTGGAAGAGGACGAGATGCAGCCTTACGCCAGCTACAC | | intracellular |
| CGAGAAGAACAACCCTCTGTACGACACCACCAACAAAGTGAAGGCC | | signaling domain |
| AGCGAGGCCCTCCAGAGCGAGGTTGACACAGATCTGCACACCCTG | | |
| CACCGGTGGTGCAGCAACAAGAAAAACGCCGCCGTGATGGACCAAG | 481 | KIR2DL1 |
| AGAGCGCCGGAAATAGAACCGCCAACAGCGAGGATAGCGACGAGCA | | intracellular |
| GGACCCTCAAGAAGTGACCTACACACAGCTGAACCACTGCGTGTTC | | signaling domain |
| ACCCAGCGGAAGATCACCAGACCTAGCCAGCGGCCTAAGACACCAC | | |
| CTACCGACATCATCGTGTACACCGAGCTGCCCAACGCCGAGAGCAG | | |
| ATCCAAGGTCGTGTCCTGTCCT | | |
| ATGACCGACAGCGTGATCTACAGCATGCTCGAGCTGCCTACAGCCA | 482 | KLRG1 |
| CACAGGCCCAGAATGATTACGGCCCTCAGCAGAAGTCCAGCTCCAG | | intracellular |
| CAGACCTAGCTGTAGCTGTCTTGGCTCCGGC | | signaling domain |
| CACCGGCAGAACCAGATCAAGCAGGGCCCTCCTAGAAGCAAGGACG | 483 | LAIR1 |
| AGGAACAGAAGCCTCAGCAGAGGCCTGATCTGGCCGTGGACGTGCT | | intracellular |
| GGAAAGAACAGCCGATAAGGCCACCGTGAACGGCCTGCCTGAGAAG | | signaling domain |
| GACAGAGAGACAGACACATCTGCCCTGGCCGCTGGCAGCTCCCAAG | | |
| AAGTGACATACGCCCAGCTGGACCACTGGGCCCTGACACAGAGAAC | | |
| TGCCAGAGCTGTGTCCCCTCAGAGCACCAAACCTATGGCCGAGAGC | | |
| ATCACCTATGCCGCCGTGGCCAGACAT | | |
| CTGCGGCACAGAAGGCAGGGCAAGCACTGGACAAGCACCCAGAGAA | 484 | LIR2 intracellular |
| AGGCCGATTTTCAGCACCCTGCTGGCGCCGTTGGACCTGAGCCTAC | | signaling domain |
| AGATAGAGGACTGCAGTGGCGGTCTAGCCCTGCTGCCGATGCTCAA | | |
| GAGGAAAACCTGTACGCCGCCGTGAAGGACACCCAACCTGAAGATG | | |
| GCGTGGAAATGGACACCAGAGCCGCCGCATCTGAAGCCCCTCAGGA | | |
| TGTGACATACGCCCAGCTGCATAGCCTGACACTGAGGCGGAAAGCC | | |
| ACAGAGCCTCCACCTAGCCAAGAGAGAGAGCCTCCTGCCGAGCCTA | | |
| GCATCTATGCCACACTGGCCATTCAC | | |
| CGGAGACAGAGACACAGCAAGCACAGAACCAGCGACCAGAGAAAGA | 485 | LIR3 intracellular |
| CCGACTTCCAGAGGCCTGCTGGCGCCGCTGAGACAGAGCCTAAAGA | | signaling domain |
| TAGAGGACTGCTGCGGAGAAGCAGCCCTGCTGCCGATGTGCAAGAG | | |
| GAAAATCTGTACGCCGCCGTGAAGGACACCCAGAGCGAGGATAGAG | | |
| TGGAACTGGACAGCCAGTCTCCTCACGACGAAGATCCTCAGGCCGT | | |
| GACATACGCCCCTGTGAAGCACAGCAGCCCTCGGAGAGAAATGGCC | | |
| TCTCCACCTTCTAGCCTGAGCGGCGAGTTCCTGGACACCAAGGACA | | |
| GACAGGTGGAAGAGGACCGGCAGATGGATACAGAAGCTGCCGCCTC | | |
| TGAAGCCAGCCAGGATGTGACATATGCCCAGCTGCATAGCCTGACA | | |
| CTGCGGCGGAAAGCTACAGAGCCTCCTCCATCTCAAGAGGGCGAGC | | |
| CTCCAGCCGAGCCTAGCATCTATGCCACACTGGCCATTCAC | | |
| CAGCATTGGAGACAGGGAAAGCACAGAACACTGGCCCAGCGGCAGG | 486 | LIR5 intracellular |
| CCGATTTCCAAAGACCTCCAGGTGCCGCCGAACCTGAGCCTAAAGA | | signaling domain |
| TGGTGGCCTGCAGCGGAGATCTTCTCCTGCTGCCGATGTGCAGGGC | | |
| GAGAATTTTTGTGCCGCCGTGAAGAACACCCAGCCTGAGGATGGCG | | |
| TGGAAATGGACACCAGACAGAGCCCTCACGACGAGGATCCACAGGC | | |
| CGTGACATACGCCAAAGTGAAGCACAGCCGGCCTCGGAGAGAAATG | | |
| GCTAGCCCTCCAAGTCCTCTGAGCGGCGAGTTCCTGGACACCAAAG | | |
| ACAGACAGGCCGAAGAGGACCGGCAGATGGATACAGAAGCTGCCGC | | |
| CTCTGAAGCCCCTCAGGATGTGACATATGCCCAGCTGCATAGCTTC | | |
| ACCCTGCGGCAGAAAGCCACAGAGCCTCCACCTTCTCAAGAGGGCG | | |
| CTTCTCCAGCCGAGCCATCTGTGTATGCCACACTGGCCATTCAC | | |
| AAGCTGCAGCGGAGATGGAAGAGAACACAGAGCCAGCAGGGCCTGC | 487 | SIGLEC-2 |
| AAGAGAATAGCAGCGGCCAGAGCTTCTTCGTGCGGAACAAGAAAGT | | intracellular |
| GCGGAGAGCCCCTCTGTCTGAGGGACCTCATAGCCTGGGCTGTTAC | | signaling domain |
| AACCCCATGATGGAAGATGGCATCAGCTACACCACACTGCGGTTCC | | |
| CCGAGATGAACATCCCTAGAACAGGCGACGCCGAGAGCAGCGAAAT | | |
| GCAAAGACCTCCTCCTGACTGCGACGACACCGTGACATATAGCGCC | | |
| CTGCACAAGAGACAAGTGGGCGACTACGAGAACGTGATCCCTGACT | | |
| TCCCTGAGGACGAGGGCATCCACTACAGCGAGCTGATCCAGTTTGG | | |
| CGTGGGCGAAAGACCCCAGGCTCAAGAGAATGTGGACTACGTGATC | | |
| CTGAAGCAC | | |
| AAGATCCTGCCTAAGCGGCGCACCCAGACCGAGACTCCTAGACCTA | 488 | SIGLEC-10 |
| GATTCAGCCGGCACAGCACCATCCTGGACTACATCAACGTGGTGCC | | intracellular |
| TACCGCCGGACCACTGGCTCAGAAGAGAAACCAGAAGGCCACACCT | | signaling domain |
| AACAGCCCCAGAACACCTCTTCCACCTGGCGCACCTTCTCCAGAGA | | |
| GCAAGAAGAACCAGAAGAAGCAGTACCAGCTGCCTAGCTTCCCCGA | | |
| GCCTAAGAGCAGTACACAGGCCCCTGAGAGCCAAGAGTCCCAAGAG | | |
| GAACTGCACTACGCCACACTGAACTTCCCCGGCGTCAGACCTAGAC | | |
| CTGAGGCCAGAATGCCTAAGGGCACCCAGGCCGATTACGCCGAAGT | | |
| GAAGTTTCAA | | |

In some embodiments, an iCAR includes a LIR1 intracellular inhibitory domain. In some embodiments, an iCAR includes a LIR1 intracellular inhibitory domain having the amino acid sequence LRHRRQGKHWTSTQRKADFQHPAGA VGPEPTDRGLQWRSSPAADAQEENLYAAVKH TQPEDGVEMDTRSPHDEDPQAVTYAEVKHSRPRREMASPPSPLSGEFLDTKDRQAEED RQMDTEAAASEAPQDVTYAQLHSLTLRREATEPPPSQEGPSPAVPSIYATLAIH (SEQ ID NO: 285). In some embodiments, an iCAR includes a LIR1 intracellular inhibitory domain encoded by a polynucleotide sequence comprising the sequence CTGCGGCACAGAAGGCAGGGCAAGCACTGGACAAGCACCCAGAGAAAGGCCGACT TTCAGCATCCTGCTGGCGCCGTTGGACCTGAGCCTACAGATAGAGGACTGCAGTGG CGGTCTAGCCCTGCCGCTGATGCCCAAGAGGAAAATCTTTACGCCGCCGTGAAGCA CACCCAGCCTGAGGATGGCGTGGAAATGGACACCAGATCTCCCCACGATGAGGACC CTCAGGCCGTGACATACGCAGAAGTGAAGCACTCCAGACCTCGGAGAGAGATGGC AAGCCCTCCATCTCCTCTGAGCGGCGAGTTCCTGGACACCAAAGACAGACAGGCCG AAGAGGACAGACAGATGGATACCGAAGCCGCCGCTTCTGAAGCCCCACAGGATGT GACATATGCCCAGCTGCATAGCCTGACACTGCGGAGAGAAGCCACAGAGCCTCCAC CTTCTCAAGAAGGCCCATCTCCTGCCGTGCCTTCCATCTATGCCACTCTGGCCATTC AC (SEQ ID NO: 286). In some embodiments, an iCAR includes a LIR1 intracellular inhibitory domain encoded by a polynucleotide sequence that is at least 90%, at least 91%, at least 92%, at least 93%, at least 94%, at least 95%, at least 96%, at least 97%, at least 98%, or at least 99% identical to

(SEQ ID NO: 286)
CTGCGGCACAGAAGGCAGGGCAAGCACTGGACAAGCACCCAGAGAAAGGC
CGACTTTCAGCATCCTGCTGGCGCCGTTGGACCTGAGCCTACAGATAGAG
GACTGCAGTGGCGGTCTAGCCCTGCCGCTGATGCCCAAGAGGAAAATCTT
TACGCCGCCGTGAAGCACACCCAGCCTGAGGATGGCGTGGAAATGGACAC
CAGATCTCCCCACGATGAGGACCCTCAGGCCGTGACATACGCAGAAGTGA
AGCACTCCAGACCTCGGAGAGAGATGGCAAGCCCTCCATCTCCTCTGAGC
GGCGAGTTCCTGGACACCAAAGACAGACAGGCCGAAGAGGACAGACAGAT
GGATACCGAAGCCGCCGCTTCTGAAGCCCCACAGGATGTGACATATGCCC
AGCTGCATAGCCTGACACTGCGGAGAGAAGCCACAGAGCCTCCACCTTCT
CAAGAAGGCCCATCTCCTGCCGTGCCTTCCATCTATGCCACTCTGGCCAT
TCAC.

In some embodiments, the one or more intracellular inhibitory co-signaling domains comprise one or more non-ITIM scaffold proteins, or a fragment(s) thereof. In some embodiments, the one or more non-ITIM scaffold proteins, or fragments thereof, are selected from GRB-2, Dok-1, Dok-2, SLAP, LAG3, HAVR, GITR, and PD-L1.

The inhibitory intracellular signaling domain can further include an enzymatic inhibitory domain. In some embodiments, the enzymatic inhibitory domain comprises an enzyme catalytic domain. In some embodiments, the enzyme catalytic domain is derived from an enzyme selected from the group consisting of: CSK, SHP-1, PTEN, CD45, CD148, PTP-MEG1, PTP-PEST, c-CBL, CBL-b, PTPN22, LAR, PTPH1, SHIP-1, and RasGAP. Examples of enzymatic regulation of signaling is described in more detail in Pavel Otáhal et al. (Biochim Biophys Acta. 2011 February; 1813 (2): 367-76), Kosugi A., et al. (Involvement of SHP-1 tyrosine phosphatase in TCR-mediated signaling pathways in lipid rafts, Immunity, 2001 June; 14 (6): 669-80), and Stanford, et al. (Regulation of TCR signaling by tyrosine phosphatases: from immune homeostasis to autoimmunity, Immunology, 2012 September; 137 (1): 1-19), each of which is incorporated herein by reference for all purposes.

In some embodiments, the intracellular signaling domain of a CAR of the present disclosure can comprise a primary signaling domain regulates primary activation of the TCR complex either in a stimulatory way or in an inhibitory way. Primary intracellular signaling domains that act in a stimulatory manner may contain signaling motifs which are known as immunoreceptor tyrosine-based activation motifs (ITAMs). Examples of suitable ITAM-containing primary intracellular signaling domains that that may be used in the CARs of the present disclosure include, without limitation, those of CD3-zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d.

In some embodiments, a CAR of the present disclosure comprises an intracellular signaling domain, e.g., a primary signaling domain of CD3-zeta polypeptide. A CD3-zeta polypeptide of the present disclosure may have an amino acid sequence that is at least 85%, at least 90%, at least 91%, at least 92%, at least 93%, at least 94%, at least 95%, at least 96%, at least 97%, at least 98%, at least 99%, or 100% homologous to the sequence of NCBI Reference No. NP_932170 or NP_001106864.2, or fragments thereof. In some embodiments, the CD3-zeta polypeptide may comprise one conservative amino acid substitution, up to two conservative amino acid substitutions, or up to three conservative amino acid substitutions. In some embodiments, the polypeptide can have an amino acid sequence that is a consecutive portion of NCBI Reference No. NP_932170 or NP_001106864.2 that is at least 20, at least 30, at least 40, at least 50, at least 60, at least 70, at least 80, at least 90, at least 100, at least 110, at least 120, at least 130, at least 140, at least 150, or at least 160, at least 170, or at least 180 amino acids in length.

In other embodiments, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In one embodiment, a primary signaling domain comprises one, two, three, four or more ITAM motifs.

In some embodiments, the intracellular signaling domain of a CAR of the present disclosure can comprise the CD3-zeta signaling domain by itself or it can be combined with any other desired intracellular signaling domain(s) useful in the context of a CAR of the present disclosure. For example, the intracellular signaling domain of the CAR can comprise a CD3-zeta chain portion and a costimulatory signaling domain. The costimulatory signaling domain may refer to a portion of the CAR comprising the intracellular domain of a costimulatory molecule. A costimulatory molecule of the present disclosure is a cell surface molecule other than an antigen receptor or its ligands that may be required for an efficient response of lymphocytes to an antigen. Examples of suitable costimulatory molecules include, without limitation, CD97, CD2, ICOS, CD27, CD154, CD8, OX40, 4-1BB, CD28, ZAP40, CD30, GITR, HVEM, DAP10, DAP12, MyD88, 2B4, CD40, PD-1, lymphocyte function-associated antigen-1 (LFA-1), CD7, LIGHT, NKG2C, B7-H3, and a ligand that specifically binds with CD83, an MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, CDS, ICAM-1, (CD11a/CD18), BAFFR, KIRDS2, SLAMF7, NKp80 (KLRF1), NKp44, NKp30, NKp46, CD19, CD4, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and the like.

In some embodiments, aCAR intracellular signaling domains include a CD28 co-stimulatory domain. In some embodiments, aCAR intracellular signaling domains include a CD28 co-stimulatory domain having the amino acid sequence RSKRSRLLHSDYMNMTPRRPGPTRKHYQPYAPPRDFAAYRS (SEQ ID NO: 287). In some embodiments, aCAR intracellular signaling domains include a CD28 co-stimulatory domain encoded by a polynucleotide sequence comprising the sequence AGAAGCAAGCGGAGCAGACTGCTGCACAGCGACTACATGAACATGACCCCTAGAC GGCCCGGACCTACCAGAAAGCACTACCAGCCTTACGCTCCTCCTAGAGATTTCGCC GCCTACCGGTCC (SEQ ID NO: 288). In some embodiments, aCAR intracellular signaling domains include a CD28 co-stimulatory domain encoded by a polynucleotide sequence that is at least 90%, at least 91%, at least 92%, at least 93%, at least 94%, at least 95%, at least 96%, at least 97%, at least 98%, or at least 99% identical to

(SEQ ID NO: 288)
AGAAGCAAGCGGAGCAGACTGCTGCACAGCGACTACATGAACATGACCCC
TAGACGGCCCGGACCTACCAGAAAGCACTACCAGCCTTACGCTCCTCCTA
GAGATTTCGCCGCCTACCGGTCC.

In some embodiments, aCAR intracellular signaling domains include a CD35 signaling domain. In some embodiments, aCAR intracellular signaling domains include a CD35 signaling domain having the amino acid sequence RVKFSRSADAPAYKQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR (SEQ ID NO: 289). In some embodiments, aCAR intracellular signaling domains include a CD35 signaling domain encoded by a polynucleotide sequence having the sequence AGAGTGAAGTTCAGCAGGAGCGCAGACGCCCCCGCGTACAAGCAGGGCCAGAACC AGCTCTATAACGAGCTCAATCTAGGACGAAGAGAGGAGTACGATGTTTTGGACAAG AGACGTGGCCGGGACCCTGAGATGGGGGGAAAGCCGAGAAGGAAGAACCCTCAGG AAGGCCTGTACAATGAACTGCAGAAAGATAAGATGGCGGAGGCCTACAGTGAGAT TGGGATGAAAGGCGAGCGCCGGAGGGGCAAGGGGCACGATGGCCTTTACCAGGGT CTCAGTACAGCCACCAAGGACACCTACGACGCCCTTCACATGCAGGCCCTGCCCCC TCGC (SEQ ID NO: 290). In some embodiments, aCAR intracellular signaling domains include a CD3ζ signaling domain encoded by a polynucleotide sequence that is at least 90%, at least 91%, at least 92%, at least 93%, at least 94%, at least 95%, at least 96%, at least 97%, at least 98%, or at least 99% identical to

(SEQ ID NO: 290)
AGAGTGAAGTTCAGCAGGAGCGCAGACGCCCCCGCGTACAAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC.

In some embodiments, aCAR intracellular signaling domains include a CD28 co-stimulatory domain and a CD3ζ signaling domain. In some embodiments, aCAR intracellular signaling domains include a CD28 co-stimulatory domain having the amino acid sequence RSKRSRLLHSDYMNMTPRRPGPTRKHYQPYAPPRDFAAYRS (SEQ ID NO: 287) and a CD3ζ signaling domain having the amino acid sequence

(SEQ ID NO: 289)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

In some embodiments, the intracellular signaling sequences within the cytoplasmic portion of a CAR of the present disclosure may be linked to each other in a random or specified order. In some embodiments, a short oligopeptide or polypeptide linker, for example, between 2 amino acids and 10 amino acids (e.g., 2 amino acids, 3 amino acids, 4 amino acids, 5 amino acids, 6 amino acids, 7 amino acids, 8 amino acids, 9 amino acids, or 10 amino acids) in length may form the linkage between intracellular signaling sequences. In one embodiment, a glycine-serine doublet can be used as a suitable linker. In one embodiment, a single ammo acid, e.g., an alanine or a glycine, can be used as a suitable linker.

In some embodiments, the intracellular signaling domain comprises two or more costimulatory signaling domains, e.g., two costimulatory signaling domains, three costimulatory signaling domains, four costimulatory signaling domains, five costimulatory signaling domains, six costimulatory signaling domains, seven costimulatory signaling domains, eight costimulatory signaling domains, nine costimulatory signaling domains, 10 costimulatory signaling domains, or more costimulatory signaling domains. In one embodiment, the intracellular signaling domain comprises two costimulatory signaling domains. In some embodiments, the two or more costimulatory signaling domains are separated by a linker of the present disclosure (e.g., any of the linkers described in Table 6). In one embodiment, the linker is a glycine residue. In another embodiment, the linker is an alanine residue.

In some embodiments, a cell of the present disclosure expresses a CAR that includes an antigen-binding domain, a transmembrane domain, a primary signaling domain, and one or more costimulatory signaling domains.

In some embodiments, the transmembrane domain is derived from the same protein as one of the one or more intracellular signaling domains. In some embodiments, the CAR is an inhibitory CAR and includes a transmembrane domain and at least one intracellular inhibitory co-signaling domain each derived from a protein selected from PD-1, CTLA4, TIGIT, BTLA, and LIR1 (LILRB1), TIM3, KIR3DL1, NKG2A, LAG3, SLAP1, SLAP2, Dok-1, Dok-2, LAIR1, GRB-2, CD200R, SIRPα, HAVR, GITR, PD-L1, KIR2DL1, KIR2DL2, KIR2DL3, KIR3DL2, CD94, KLRG-1, CEACAM1, LIR2, LIR3, LIR5, SIGLEC-2, and SIGLEC-10.

In some embodiments, the transmembrane domain is derived from a first protein and the one or more intracellular signaling domains are derived from a second protein that are distinct from the first protein.

### Natural Killer Cell Receptor (NKR) CARs

In some embodiments, a CAR of the present disclosure comprises one or more components of a natural killer cell receptor (NKR), thereby forming an NKR-CAR. The NKR component may be a transmembrane domain, a hinge domain, or a cytoplasmic domain from any suitable natural killer cell receptor, including without limitation, a killer cell immunoglobulin-like receptor (KIR),such as KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2 DPI, and KIRS DPI; a natural cytotoxicity receptor (NCR), such as NKp30, NKp44, NKp46; a signaling lymphocyte activation molecule (SLAM) family of immune cell receptor, such as CD48, CD229, 2B4, CD84, NTB-A, CRACC, BLAME, and CD2F-10; an Fc receptor (FcR), such as CD16, and CD64; and an Ly49 receptor, such as LY49A and LY49C. In some embodiments, the NKR-CAR may interact with an adaptor molecule or intracellular signaling domain, such as DAP12. Exemplary configurations and sequences of CARs comprising NKR components are described in International Patent Publication WO2014/145252, published Sep. 18, 2014.

### Immunoresponsive Cells

Certain aspects of the present disclosure relate to a cell, such as an immunoresponsive cell, that has been genetically engineered to comprise one or more chimeric receptors of the present disclosure or one or more nucleic acids encoding such chimeric receptors, and to methods of using such cells for treating myeloid malignancies (e.g., AML).

In some embodiments, the cell is a mammalian cell. In some embodiments, the mammalian cell is a primary cell. In some embodiments, the mammalian cell is a cell line. In some embodiments, the mammalian cell, a bone marrow cell, a blood cell, a skin cell, bone cell, a muscle cell, a neuronal cell, a fat cell, a liver cell, or a heart cell. In some embodiments, the cell is a stem cell. Exemplary stem cells include, without limitation embryonic stem cells (ESCs), induced pluripotent stem cells (iPSCs), adult stem cells, and tissue-specific stem cells, such as hematopoietic stem cells (blood stem cells), mesenchymal stem cells (MSC), neural stem cells, epithelial stem cells, or skin stem cells. In some embodiments, the cell is a cell that is derived or differentiated from a stem cell of the present disclosure. In some embodiments, the cell is an immune cell. Immune cells of the present disclosure may be isolated or differentiated from a stem cell of the present disclosure (e.g., from an ESC or iPSC). Exemplary immune cells include, without limitation, T cells (e.g., helper T cells, cytotoxic T cells, memory T cells, regulatory T cells, natural killer T cells, alpha beta T cells, and gamma delta T cells), B cells, natural killer (NK) cells, dendritic cells, myeloid cells, macrophages, and monocytes. In some embodiments, the cell is a neuronal cell. Neuronal cells of the present disclosure may be isolated or differentiated from a stem cell of the present disclosure (e.g., from an ESC or iPSC). Exemplary neuronal cells include, without limitation, neural progenitor cells, neurons (e.g., sensory neurons, motor neurons, cholinergic neurons, GABAergic neurons, glutamatergic neurons, dopaminergic neurons, or serotonergic neurons), astrocytes, oligodendrocytes, and microglia.

In some embodiments, the cell is an immunoresponsive cell. Immunoresponsive cells of the present disclosure may be isolated or differentiated from a stem cell of the present disclosure (e.g., from an ESC or iPSC). Exemplary immunoresponsive cells of the present disclosure include, without limitation, cells of the lymphoid lineage. The lymphoid lineage, comprising B cells, T cells, and natural killer (NK) cells, provides for the production of antibodies, regulation of the cellular immune system, detection of foreign agents in the blood, detection of cells foreign to the host, and the like. Examples of immunoresponsive cells of the lymphoid lineage include, without limitation, T cells, Natural Killer (NK) cells, embryonic stem cells, pluripotent stem cells, and induced pluripotent stem cells (e.g., those from which lymphoid cells may be derived or differentiated). T cells can be lymphocytes that mature in the thymus and are chiefly responsible for cell-mediated immunity. T cells are involved in the adaptive immune system. In some embodiments, T cells of the present disclosure can be any type of T cells, including, without limitation, T helper cells, cytotoxic T cells, memory T cells (including central memory T cells, stem-cell-like memory T cells (or stem-like memory T cells), and two types of effector memory T cells: e.g., T_{EM} cells and T_{EMRA} cells, regulatory T cells (also known as suppressor T cells), natural killer T cells, mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of one or more chimeric receptors, such as a chimeric TCRs or CARs.

Natural killer (NK) cells can be lymphocytes that are part of cell-mediated immunity and act during the innate immune response. NK cells do not require prior activation in order to perform their cytotoxic effect on target cells.

In some embodiments, an immunoresponsive cell of the present disclosure is a T cell. T cells of the present disclosure may be autologous, allogeneic, or derived in vitro from engineered progenitor or stem cells.

In some embodiments, an immunoresponsive cell of the present disclosure is a universal T cell with deficient TCR-αβ. Methods of developing universal T cells are described in the art, for example, in Valton et al., Molecular Therapy (2015); 23 9, 1507-1518, and Torikai et al., Blood 2012 119:5697-5705.

In some embodiments, an immunoresponsive cell of the present disclosure is an isolated immunoresponsive cell comprising one or more chimeric receptors of the present disclosure. In some embodiments, the immunoresponsive cell comprises one or more, two or more, three or more, four or more, five or more, six or more, seven or more, eight or more, nine or more, or ten or more chimeric receptors of the present disclosure.

In some embodiments, an immunoresponsive cell is a T cell. In some embodiments, an immunoresponsive cell is a Natural Killer (NK) cell.

In some embodiments, an immunoresponsive cell expresses or is capable of expressing an immune receptor. Immune receptors generally are capable of inducing signal transduction or changes in protein expression in the immune receptor-expressing cell that results in the modulation of an immune response upon binding to a cognate ligand (e.g., regulate, activate, initiate, stimulate, increase, prevent, attenuate, inhibit, reduce, decrease, inhibit, or suppress an immune response). For example, when CD3 chains present in a TCR/CAR cluster in response to ligand binding, an immunoreceptor tyrosine-based activation motifs (ITAMs)-meditated signal transduction cascade is produced. Specifically, in certain embodiments, when an endogenous TCR, exogenous TCR, chimeric TCR, or a CAR (specifically an activating CAR) binds their respective antigen, a formation of an immunological synapse occurs that includes clustering of many molecules near the bound receptor (e.g., CD4 or CD8, CD3γ/δ/ε/ζ, etc.). This clustering of membrane bound signaling molecules allows for ITAM motifs contained within the CD3 chains to become phosphorylated that in turn can initiate a T cell activation pathway and ultimately activates transcription factors, such as NF-κB and AP-1. These transcription factors are capable of inducing global gene expression of the T cell to increase IL-2 production for proliferation and expression of master regulator T cell proteins in order to initiate a T cell mediated immune response, such as cytokine production and/or T cell mediated killing.

### Cells Expressing Multiple Chimeric Receptors

In some embodiments, a cell of the present disclosure (e.g., an immunoresponsive cell) comprises two or more chimeric receptors of the present disclosure. In some embodiments, the cell comprises two or more chimeric receptors, wherein one of the two or more chimeric receptors is a chimeric inhibitory receptor. In some embodiments, the cell comprises three or more chimeric receptors, wherein at least one of the three or more chimeric receptors is a chimeric inhibitory receptor. In some embodiments, the cell comprises four or more chimeric receptors, wherein at least one of the four or more chimeric receptors is a chimeric inhibitory receptor. In some embodiments, the cell comprises five or more chimeric receptors, wherein at least one of the five or more chimeric receptors is a chimeric inhibitory receptor.

In some embodiments, each of the two or more chimeric receptors comprise a different antigen-binding domain, e.g., that binds to the same antigen or to a different antigen. In some embodiments each antigen bound by the two or more chimeric receptors are expressed on the same cell, such as a myeloid cell type (e.g., same AML cell type). In some embodiments each antigen bound by the two or more chimeric receptors is an AML-associated antigen (e.g., FLT3, CD33, CD123, CLEC12A, CXCR4, EphA3, etc.).

In embodiments where a cell of the present disclosure (e.g., an immunoresponsive cell) expresses two or more distinct chimeric receptors, the antigen-binding domain of each of the different chimeric receptors may be designed such that the antigen-binding domains do not interact with one another. For example, a cell of the present disclosure (e.g., an immunoresponsive cell) expressing a first chimeric receptor (e.g., an EMCN-specific chimeric receptor) and a second chimeric receptor may comprise a first chimeric receptor that comprises an antigen-binding domain that does not form an association with the antigen-binding domain of the second chimeric receptor. For example, the antigen-binding domain of the first chimeric receptor may comprise an antibody fragment, such as an scFv, while the antigen-binding domain of the second chimeric receptor may comprise a VHH.

Without wishing to be bound by theory, it is believed that in cells having a plurality of chimeric membrane embedded receptors that each comprise an antigen-binding domain, interactions between the antigen-binding domains of each of the receptors can be undesirable, because such interactions may inhibit the ability of one or more of the antigen-binding domains to bind their cognate antigens. Accordingly, in embodiments where cells of the present disclosure (e.g., immunoresponsive cells) express two or more chimeric receptors, the chimeric receptors comprise antigen-binding domains that minimize such inhibitory interactions. In one embodiment, the antigen-binding domain of one chimeric receptor comprises an scFv and the antigen-binding domain of the second chimeric receptor comprises a single VH domain, e.g., a camelid, shark, or lamprey single VH domain, or a single VH domain derived from a human or mouse sequence.

In some embodiments, when present on the surface of a cell, binding of the antigen-binding domain of the first chimeric receptor to its cognate antigen is not substantially reduced by the presence of the second chimeric receptor. In some embodiments, binding of the antigen-binding domain of the first chimeric receptor to its cognate antigen in the presence of the second chimeric receptor is 85%, 90%, 95%, 96%, 97%, 98%, or 99% of binding of the antigen-binding domain of the first chimeric receptor to its cognate antigen in the absence of the second chimeric receptor. In some embodiments, when present on the surface of a cell, the antigen-binding domains of the first chimeric receptor and the second chimeric receptor associate with one another less than if both were scFv antigen-binding domains. In some embodiments, the antigen-binding domains of the first chimeric receptor and the second chimeric receptor associate with one another 85%, 90%, 95%, 96%, 97%, 98%, or 99% less than if both were scFv antigen-binding domains.

### Chimeric Inhibitory Receptors

In some embodiments, a cell of the present disclosure (e.g., an immunoresponsive cell) comprises one or more chimeric inhibitory receptors of the present disclosure. In some embodiments, each of the one or more chimeric inhibitory receptors comprises an antigen-binding domain that binds an antigen generally expressed on normal cells (e.g., cells generally considered to be healthy) but not on tumor cells, such as AML cells. In some embodiments, a chimeric inhibitory receptor includes an antigen-binding domain that binds EMCN (e.g., an EMCN-specific antigen-binding domain having one or more of the amino acid sequences listed in Table 1).

In some embodiments, the one or more chimeric inhibitory receptors bind antigens that are expressed on a non-tumor cell derived from a tissue selected from the group consisting of brain, neuronal tissue, endocrine, bone, bone marrow, immune system, endothelial tissue, muscle, lung, liver, gallbladder, pancreas, gastrointestinal tract, kidney, urinary bladder, male reproductive organs, female reproductive organs, adipose, soft tissue, and skin.

In some embodiments, a chimeric inhibitory receptor (e.g., an EMCN-specific chimeric inhibitory receptor) may be used, for example, with one or more activating chimeric receptors (e.g., activating chimeric TCRs or CARs) expressed on a cell of the present disclosure (e.g., an immunoresponsive cell) as NOT-logic gates to control, modulate, or otherwise inhibit one or more activities of the one or more activating chimeric receptors. In some embodiments, a chimeric inhibitory receptor of the present disclosure may inhibit one or more activities of a cell of the present disclosure (e.g., an immunoresponsive cell).

In some embodiments, a cell of the present disclosure comprises one or more chimeric inhibitory receptors of the present disclosure and further comprises a tumor-targeting chimeric receptor that binds to one or more tumor-associated antigens. In some embodiments, the one or more tumor-associated antigens include an AML-associated antigen. In some embodiments, the one or more tumor-associated antigens include CD33. In some embodiments, the one or more tumor-associated antigens include FLT3. In some embodiments, the one or more tumor-associated antigens include CD33 and FLT3.

### Co-Stimulatory Ligands

In some embodiments, a cell of the present disclosure (e.g., an immunoresponsive cell) can further include one or more recombinant or exogenous co-stimulatory ligands. For example, the cell can be further transduced with one or more co-stimulatory ligands, such that the cell co-expresses or is induced to co-express one or more chimeric receptors of the present disclosure (e.g., the EMCN-specific, FLT3-specific, and/or CD33-specific CARs described herein) and one or more co-stimulatory ligands. Without wishing to be bound by theory, it is believed that the interaction between the one or more chimeric receptors and the one or more co-stimulatory ligands may provide a non-antigen-specific signal important for full activation of the cell. Examples of suitable co-stimulatory ligands include, without limitation, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. Examples of suitable TNF superfamily members include, without limitation, nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LTa), lymphotoxin-beta (LTP), CD257/B cell-activating factor (B AFF)/Bly s/THANK/Tall-1, glucocorticoid-induced TNF Receptor ligand (GITRL), and TNF-related apoptosis-inducing ligand (TRAIL), LIGHT (TNFSF 14). The immunoglobulin (Ig) superfamily is a large group of cell surface and soluble proteins that are involved in the recognition, binding, or adhesion processes of cells. These proteins share structural features with immunoglobulins and possess an immunoglobulin domain (fold). Examples of suitable immunoglobulin superfamily ligands include, without limitation, CD80 and CD86, both ligands for CD28, PD-L1/(B7-H1) that ligands for PD-1. In certain embodiments, the one or more co-stimulatory ligands are selected from 4-1BBL, CD80, CD86, CD70, OX40L, CD48, TNFRSF14, PD-L1, and combinations thereof.

### Chemokine Receptor

In some embodiments, a cell of the present disclosure (e.g., an immunoresponsive cell) comprises one or more chimeric receptors (e.g., the EMCN-specific, FLT3-specific, and/or CD33-specific CARs described herein) and may further include one or more chemokine receptors. For example, transgenic expression of chemokine receptor CCR2b or CXCR2 in cells, such as T cells, enhances trafficking to CCL2-secreting or CXCL1-secreting solid tumors (Craddock et al, J Immunother. 2010 October; 33 (8): 780-8 and Kershaw et al. Hum Gene Ther. 2002 Nov. 1; 13 (16): 1971-80). Without wishing to be bound by theory, it is believed that chemokine receptors expressed on chimeric receptor-expressing cells of the present disclosure may recognize chemokines secreted by tumors and improve targeting of the cell to the tumor, which may facilitate the infiltration of the cell to the tumor and enhance the antitumor efficacy of the cell. Chemokine receptors of the present disclosure may include a naturally occurring chemokine receptor, a recombinant chemokine receptor, or a chemokine-binding fragment thereof. Examples of suitable chemokine receptors that may expressed on a cell of the present disclosure include, without limitation, a CXC chemokine receptor, such as CXCR1, CXCR2, CXCR3, CXCR4, CXCR5, CXCR6, or CXCR7; a CC chemokine receptor, such as CCR1, CCR2, CCR3, CCR4, CCR5, CCR6, CCR7, CCR8, CCR9, CCR 10, or CCR11; a CX3C chemokine receptor, such as CX3CR1; an XC chemokine receptor, such as XCR1; and chemokine-binding fragments thereof. In some embodiments, the chemokine receptor to be expressed on the cell is chosen based on the chemokines secreted by the tumor.

### Chimeric Receptor Regulation

Some embodiments of the present disclosure relate to regulating one or more chimeric receptor activities of chimeric receptor-expressing cells of the present disclosure (e.g., the EMCN-specific CARs described herein). There are several ways chimeric receptor activities can be regulated. In some embodiments, a regulatable chimeric receptor, wherein one or more chimeric receptor activities can be controlled, may be desirable to optimize the safety and/or efficacy of the chimeric receptor therapy. For example, inducing apoptosis using a caspase fused to a dimerization domain (see, e.g., Di et al., N Engl. J. Med. 2011 Nov. 3; 365 (18): 1673-1683) can be used as a safety switch in the chimeric receptor therapy. In some embodiments, a chimeric receptor-expressing cell of the present disclosure can also express an inducible Caspase-9 (iCaspasc-9) that, upon administration of a dimerizer drug, such as rimiducid (IUPAC name: [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl)butanoyl]piperidine-2-carbonyl]oxypropyl]phenoxy]acetyl]amino]ethylamino]-2-oxoethoxy]phenyl]propyl] (2S)-1-[(2S)-2-(3,4,5-trimethoxyphenyl)butanoyl]piperidine-2-carboxylate), induces activation of the Caspase-9 and results in apoptosis of the cells. In some embodiments, the iCaspase-9 contains a binding domain that comprises a chemical inducer of dimerization (CID) that mediates dimerization in the presence of the CID, which results in inducible and selective depletion of the chimeric receptor-expressing cells.

Alternatively, in some embodiments a chimeric receptor of the present disclosure may be regulated by utilizing a small molecule or an antibody that deactivates or otherwise inhibits chimeric receptor activity. For example, an antibody may delete the chimeric receptor-expressing cells by inducing antibody dependent cell-mediated cytotoxicity (ADCC). In some embodiments, a chimeric receptor-expressing cell of the present disclosure may further express an antigen that is recognized by a molecule that is capable of inducing cell death by ADCC or complement-induced cell death. For example, a chimeric receptor-expressing cell of the present disclosure may further express a receptor capable of being targeted by an antibody or antibody fragment. Examples of suitable receptors that may be targeted by an antibody or antibody fragment include, without limitation, EpCAM, VEGFR, integrins (e.g., ανβ3, α4, αI¾β3, α4β7, α5β1, ανβ3, αν), members of the TNF receptor superfamily (e.g., TRAIL-R1 and TRAIL-R2), PDGF receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/IgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof.

In some embodiments, a chimeric receptor-expressing cell of the present disclosure may also express a truncated epidermal growth factor receptor (EGFR) that lacks signaling capacity but retains an epitope that is recognized by molecules capable of inducing ADCC (e.g., WO2011/056894).

In some embodiments, a chimeric receptor-expressing cell of the present disclosure further includes a highly expressing compact marker/suicide gene that combines target epitopes from both CD32 and CD20 antigens in the chimeric receptor-expressing cell, which binds an anti-CD20 antibody (e.g., rituximab) resulting in selective depletion of the chimeric receptor-expressing cell by ADCC. Other methods for depleting chimeric receptor-expressing cells of the present disclosure my include, without limitation, administration of a monoclonal anti-CD52 antibody that selectively binds and targets the chimeric receptor-expressing cell for destruction by inducing ADCC. In some embodiments, the chimeric receptor-expressing cell can be selectively targeted using a chimeric receptor ligand, such as an anti-idiotypic antibody. In some embodiments, the anti-idiotypic antibody can cause effector cell activity, such as ADCC or ADC activity. In some embodiments, the chimeric receptor ligand can be further coupled to an agent that induces cell killing, such as a toxin. In some embodiments, a chimeric receptor-expressing cell of the present disclosure may further express a target protein recognized by a cell depleting agent of the present disclosure. In some embodiments, the target protein is CD20 and the cell depleting agent is an anti-CD20 antibody. In such embodiments, the cell depleting agent is administered once it is desirable to reduce or eliminate the chimeric receptor-expressing cell. In some embodiments, the cell depleting agent is an anti-CD52 antibody.

In some embodiments, a regulated chimeric receptor comprises a set of polypeptides, in which the components of a chimeric receptor of the present disclosure are partitioned on separate polypeptides or members. For example, the set of polypeptides may include a dimerization switch that, when in the presence of a dimerization molecule, can couple the polypeptides to one another to form a functional chimeric receptor.

### Endomucin-Specific Antigen-Binding Domains

The present disclosure provides antigen-binding domains (e.g., single-chain variable fragments) that bind to endomucin (EMCN), chimeric proteins including antigen-binding domains that bind to EMCN (e.g., any of the iCARs described herein), and nucleic acids encoding such antigen-binding domains and chimeric proteins. Without wishing to be bound by theory, EMCN is a sialoglycoprotein that interferes with assembly of focal adhesion complexes and inhibits interaction between cells and extracellular matrix. EMCN-specific antigen-binding domains bind to human EMCN (e.g., Uniprot Q9ULC0, herein incorporated by reference for all purposes) or an epitope fragment thereof. EMCN can be expressed on cells generally considered to be healthy, such as healthy hematopoietic stem cells (HSCs), HSPCs, healthy multipotent progenitors (MPPs), healthy lympho-myeloid primed progenitor (LMPPs), and healthy hematopoietic progenitor cells (HPCs). EMCN can be expressed on hematopoietic stem and progenitor cells (HSPCs). EMCN can be expressed on HSCs. EMCN can be expressed on MPPs. EMCN can be expressed on LMPPs. EMCN can be expressed on HPCs. EMCN-specific antibodies have been previously described, including CBFYE-0213, V.7.C7.1, L4B1, L5F12, L10B5, L3F12, L6H3, L9H8, and L10F12, as described in Samulowitz U. et al., Am. J. Path., 2002 May, 160 (5): 1669-1681, herein incorporated by reference for all purposes.

The present disclosure provides an EMCN-specific antigen-binding domain including one or more of the amino acid sequences listed in Table 1.

TABLE 1

| | SEQ ID | |
|---|---|---|
| Amino Acid Sequence | NO: | Description |
| RYDMH | 291 | CDR-H1 of Kabat- |
| | | annotated CDR |
| GFTFSRY | 292 | CDR-H1 Version 1 of |
| | | Chothia-annotated CDR |
| GFTLSRY | 293 | CDR-H1 Version 2 of |
| | | Chothia-annotated CDR |
| GFSFSRY | 294 | CDR-H1 Version 3 of |
| | | Chothia-annotated CDR |
| GFSLSRY | 295 | CDR-H1 Version 4 of |
| | | Chothia-annotated CDR |
| VIWGNGNTHYHSALKS | 296 | CDR-H2 of Kabat- |
| | | annotated CDR |
| WGNGN | 297 | CDR-H2 of Chothia- |
| | | annotated CDR |
| RIKD | 298 | CDR-H3 (Kabat- and |
| | | Chothia-annotated CDR) |
| KSSQSLVASDENTYLN | 299 | CDR-L1 (Kabat- and |
| | | Chothia-annotated CDR) |
| QVSKLDS | 300 | CDR-L2 (Kabat- and |
| | | Chothia-annotated CDR) |
| LQGIHLPWT | 301 | CDR-L3(Kabat- and |
| | | Chothia-annotated CDR) |
| EVQLVESGGGLVQPGGSLRLSCAASGFTFSRYDMHWVRQAPG | 302 | VH 3-23 Version 1 |
| KGLEWVSVIWGNGNTHYHSALKSRFTISRDNSKNTLYLQMNS | | |
| LRAEDTAVYYCTLRIKDWGQGTMVTVSS | | |
| EVQLVESGGGLVQPGGSLRLSCAASGFTLSRYDMHWVRQAPG | 303 | VH 3-23 Version 2 |
| KGLEWVSVIWGNGNTHYHSALKSRFTISRDNSKNTLYLQMNS | | |
| LRAEDTAVYYCTLRIKDWGQGTMVTVSS | | |
| EVQLVESGGGLVQPGGSLRLSCAASGFSFSRYDMHWVRQAPG | 304 | VH 3-23 Version 3 |
| KGLEWVSVIWGNGNTHYHSALKSRFTISRDNSKNTLYLQMNS | | |
| LRAEDTAVYYCTLRIKDWGQGTMVTVSS | | |
| EVQLVESGGGLVQPGGSLRLSCAASGFSLSRYDMHWVRQAPG | 305 | VH 3-23 Version 4 |
| KGLEWVSVIWGNGNTHYHSALKSRFTISRDNSKNTLYLQMNS | | |
| LRAEDTAVYYCTLRIKDWGQGTMVTVSS | | |
| QVQLVESGGGVVQPGRSLRLSCAASGFTFSRYDMHWVRQAPG | 306 | VH 3-33 Version 1 |
| KGLEWVAVIWGNGNTHYHSALKSRFTISRDNSKNTLYLQMNS | | |
| LRAEDTAVYYCTLRIKDWGQGTMVTVSS | | |
| QVQLVESGGGVVQPGRSLRLSCAASGFTLSRYDMHWVRQAPG | 307 | VH 3-33 Version 2 |
| KGLEWVAVIWGNGNTHYHSALKSRFTISRDNSKNTLYLQMNS | | |
| LRAEDTAVYYCTLRIKDWGQGTMVTVSS | | |
| QVQLVESGGGVVQPGRSLRLSCAASGFSFSRYDMHWVRQAPG | 308 | VH 3-33 Version 3 |
| KGLEWVAVIWGNGNTHYHSALKSRFTISRDNSKNTLYLQMNS | | |
| LRAEDTAVYYCTLRIKDWGQGTMVTVSS | | |
| QVQLVESGGGVVQPGRSLRLSCAASGFSLSRYDMHWVRQAPG | 309 | VH 3-33 Version 4 |
| KGLEWVAVIWGNGNTHYHSALKSRFTISRDNSKNTLYLQMNS | | |
| LRAEDTAVYYCTLRIKDWGQGTMVTVSS | | |
| DVVMTQSPLSLPVTLGQPASISCKSSQSLVASDENTYLNWFQ | 310 | VL 2-30 |
| QRPGQSPRRLIYQVSKLDSGVPDRFSGSGSGTDFTLKISRVE | | |
| AEDVGVYYCLQGIHLPWTFGQGTKLEIK | | |

In some embodiments, the antigen-binding domain specific for EMCN includes a heavy chain variable (VH) region and a light chain variable (VL) region, wherein: the EMCN-VH includes a heavy chain complementarity determining region 1 (CDR-H1) having the amino acid sequence of RYDMH (SEQ ID NO: 291), a heavy chain complementarity determining region 2 (CDR-H2) having the amino acid sequence of VIWGNGNTHYHSALKS (SEQ ID NO: 296), and a heavy chain complementarity determining region 3 (CDR-H3) having the amino acid sequence of RIKD (SEQ ID NO: 298), and the EMCN-VL includes a light chain complementarity determining region 1 (CDR-L1) having the amino acid sequence of KSSQSLVASDENTYLN (SEQ ID NO: 299), a light chain complementarity determining region 2 (CDR-L2) having the amino acid sequence of QVSKLDS (SEQ ID NO: 300), and a light chain complementarity determining region 3 (CDR-L3) having the amino acid sequence of LOGIHLPWT (SEQ ID NO: 301), and where the amino acid sequences of the CDR-H1, the CDR-H2, the CDR-H3, the CDR-L1, the CDR-L2, and the CDR-L3 of the reference antibody are defined based on the Kabat numbering scheme.

In some embodiments, the antigen-binding domain specific for EMCN includes a heavy chain variable (VH) region and a light chain variable (VL) region, wherein: the EMCN-VH includes the amino acid sequence EVOLVESGGGLVQPGGSLRLSCAASGFTFSRYDMHWVRQAPGKGLEWVSVIWGNGNT HYHSALKSRFTISRDNSKNTLYLQMNSLRAEDTAVYYCTLRIKDWGQGTMVTVSS (SEQ ID NO: 302); and the EMCN-VL includes the amino acid sequence DVVMTQSPLSLPVTLGQPASISCKSSQSLVASDENTYLNWFQQRPGQSPRRLIYQVSKL DSGVPDRFSGSGSGTDFTLKISRVEAEDVGVYYCLQGIHLPWTFGQGTKLEIK (SEQ ID NO: 310).

In some embodiments, the antigen-binding domain specific for EMCN includes the amino acid sequence EVOLVESGGGLVQPGGSLRLSCAASGFTFSRYDMHWVRQAPGKGLEWVSVIWGNGNT HYHSALKSRFTISRDNSKNTLYLQMNSLRAEDTAVYYCTLRIKDWGQGTMVTVSSGGG GSGGGGSGGGGSDVVMTQSPLSLPVTLGQPASISCKSSQSLVASDENTYLNWFQQRPG QSPRRLIYQVSKLDSGVPDRFSGSGSGTDFTLKISRVEAEDVGVYYCLQGIHLPWTFGQ GTKLEIK (SEQ ID NO: 311). In some embodiments, the antigen-binding domain specific for EMCN is encoded by a polynucleotide sequence comprising the sequence

(SEQ ID NO: 312)
GAGGTGCAGCTGGTTGAATCTGGCGGAGGACTGGTTCAGCCTGGCGGATC
TCTGAGACTGTCTTGTGCCGCCAGCGGCTTCACCTTCAGCAGATACGATA
TGCACTGGGTCCGACAGGCCCCTGGCAAAGGACTTGAATGGGTGTCCGTG
ATCTGGGGCAACGGCAACACACACTACCACAGCGCCCTGAAGTCCCGGTT
CACCATCTCCAGAGACAACAGCAAGAACACCCTGTACCTGCAGATGAACA
GCCTGAGAGCCGAGGACACCGCCGTGTACTACTGCACCCTGAGAATCAAG
GATTGGGGCCAGGGCACCATGGTCACCGTTTCTTCTGGAGGCGGAGGATC
TGGTGGCGGAGGAAGTGGCGGAGGCGGTTCTGACGTGGTCATGACACAGA
GCCCTCTGAGCCTGCCTGTGACACTGGGACAGCCTGCCAGCATCAGCTGC
AAGTCTAGCCAGTCTCTGGTGGCCAGCGACGAGAACACCTACCTGAACTG
GTTCCAGCAGAGGCCCGGACAGTCTCCTAGACGGCTGATCTACCAGGTGT
CCAAGCTGGATAGCGGCGTGCCCGATAGATTTTCTGGCAGCGGCTCTGGC
ACCGACTTCACCCTGAAGATCAGCAGAGTGGAAGCCGAGGACGTGGGCGT
GTACTACTGTCTGCAAGGCATCCATCTGCCTTGGACCTTTGGCCAGGGCA
CAAAGCTGGAAATCAAG.

In some embodiments, the antigen-binding domain specific for EMCN is encoded by a polynucleotide sequence that is at least 90%, at least 91%, at least 92%, at least 93%, at least 94%, at least 95%, at least 96%, at least 97%, at least 98%, or at least 99% identical to

(SEQ ID NO: 312)
GAGGTGCAGCTGGTTGAATCTGGCGGAGGACTGGTTCAGCCTGGCGGATC
TCTGAGACTGTCTTGTGCCGCCAGCGGCTTCACCTTCAGCAGATACGATA
TGCACTGGGTCCGACAGGCCCCTGGCAAAGGACTTGAATGGGTGTCCGTG
ATCTGGGGCAACGGCAACACACACTACCACAGCGCCCTGAAGTCCCGGTT
CACCATCTCCAGAGACAACAGCAAGAACACCCTGTACCTGCAGATGAACA
GCCTGAGAGCCGAGGACACCGCCGTGTACTACTGCACCCTGAGAATCAAG
GATTGGGGCCAGGGCACCATGGTCACCGTTTCTTCTGGAGGCGGAGGATC
TGGTGGCGGAGGAAGTGGCGGAGGCGGTTCTGACGTGGTCATGACACAGA
GCCCTCTGAGCCTGCCTGTGACACTGGGACAGCCTGCCAGCATCAGCTGC
AAGTCTAGCCAGTCTCTGGTGGCCAGCGACGAGAACACCTACCTGAACTG
GTTCCAGCAGAGGCCCGGACAGTCTCCTAGACGGCTGATCTACCAGGTGT
CCAAGCTGGATAGCGGCGTGCCCGATAGATTTTCTGGCAGCGGCTCTGGC
ACCGACTTCACCCTGAAGATCAGCAGAGTGGAAGCCGAGGACGTGGGCGT
GTACTACTGTCTGCAAGGCATCCATCTGCCTTGGACCTTTGGCCAGGGCA
CAAAGCTGGAAATCAAG.

### FLT3 and CD33-Specific Antigen-Binding Domains

The present disclosure provides antigen-binding domains (e.g., single-chain variable fragments) that bind to FLT3 and CD33, chimeric proteins including antigen-binding domains that bind to FLT3 and CD33 (e.g., any of the aCARs described herein), and nucleic acids encoding such antigen-binding domains and chimeric proteins.

In some embodiments, chimeric receptors comprise one or more of the amino acid sequences listed in Table A1 or Table A2. In some embodiments, activating chimeric receptors (aCARS) comprise one or more of the amino acid sequences listed in Table A1 or Table A2. In some embodiments, bispecific-bivalent aCARS comprise one or more of the amino acid sequences listed in Table A1 or Table A2. Table A1 provides the variable domains of an antibody heavy chain or light chain. The CDRs were determined using the Kabat method and are in bold italic in Table A1 for each variable heavy chain or variable light chain and shown in Table A2. In some embodiments, nucleic acids encoding any of the chimeric receptors of the present disclosure comprise one or more of the nucleic acid sequences listed in Table B.

TABLE A1

| | SEQ ID | |
|---|---|---|
| Amino Acid Sequence | NO: | Description |
| EVQLVQSGAEVKKPGASVKVSCKASGYTFTSYYMHWARQAPGQ | 313 | Heavy chain variable domain |
| GLEWMGIINPSGGSTSYAQKFQGRVTMTRDTSTSTVYMELSSL | | of anti-FLT3 antibody D4-3 |
| RSEDTAVYYCARVVAAAVADYWGQGTLVTVSS | | |
| DVVMTQSPLSLPVTPGEPASISCRSSQSLLHSNGYNYLDWYLQ | 314 | Light chain variable domain |
| KPGQSPQLLIYLGSNRASGVPDRFSGSGSGTDFTLKISRVEAE | | of anti-FLT3 antibody D4-3 |
| DVGVYYCMQSLQTPFTFGPGTKVDIK | | |
| EVQLVQSGAEVKKPGSSVKVSCKASGGTFSSYAISWVRQAPGQ | 315 | Heavy chain variable domain |
| GLEWMGGIIPIFGTANYAQKFQGRVTITADKSTSTAYMELSSL | | of anti-FLT3 antibody NC7 |
| RSEDTAVYYCATFALFGFREQAFDIWGQGTTVTVSS | | |
| DIQMTQSPSSLSASVGDRVTITCRASQSISSYLNWYQQKPGKA | 316 | Light chain variable domain |
| PKLLIYAASSLQSGVPSRFSGSGSGTDFTLTISSLQPEDLATY | | of anti-FLT3 antibody NC7 |
| YCQQSYSTPFTFGPGTKVDIK | | |
| EVQLVQSGAEVKKPGASVKVSCKASGYTFTSYYMHWVRQAPGQ | 317 | Heavy chain variable domain |
| GLEWMGIINPSGGSTSYAQKFQGRVTMTRDTSTSTVYMELSSL | | of anti-FLT3 antibody EB10 |
| RSEDTAVYYCARGVGAHDAFDIWGQGTTVTVSS | | |
| DVVMTQSPLSLPVTPGEPASISCRSSQSLLHSNGNNYLDWYLQ | 318 | Light chain variable domain |
| KPGQSPQLLIYLGSNRASGVPDRFSGSGSDTDFTLQISRVEAE | | of anti-FLT3 antibody EB10 |
| DVGVYYCMQGTHPAISFGQGTRLEIK | | |
| QVQLQQPGAELVKPGASLKLSCKSSGYTFTSYWMHWVRQRPGH | 319 | Heavy chain variable domain |
| GLEWIGEIDPSDSYKDYNQKFKDKATLTVDRSSNTAYMHLSSL | | of anti-FLT3 antibody 4G8 |
| TSDDSAVYYCARAITTTPFDFWGQGTTLTVSS | | |
| DIVLTQSPATLSVTPGDSVSLSCRASQSISNNLHWYQQKSHES | 320 | Light chain variable domain |
| PRLLIKYASQSISGIPSRFSGSGSGTDFTLSINSVETEDFGVY | | of anti-FLT3 antibody 4G8 |
| FCQQSNTWPYTFGGGTKLEIKR | | |
| QVTLKESGPTLVKPTETLTLTCTLSGFSLNNARMGVSWIRQPP | 321 | Heavy chain variable domain |
| GKCLEWLAHIFSNDEKSYSTSLKNRLTISKDSSKTQVVLTMTN | | of anti-FLT3 antibody FL_39 |
| VDPVDTATYYCARIVGYGSGWYGFFDYWGQGTLVTVSS | | |
| DIQMTQSPSSLSASVGDRVTITCRASQGIRNDLGWYQQKPGKA | 322 | Light chain variable domain |
| PKRLIYAASTLQSGVPSRFSGSGSGTEFTLTISSLQPEDFATY | | of anti-FLT3 antibody FL_39 |
| YCLQHNSYPLTFGCGTKVEIK | | |
| QVTLKESGPVLVKPTETLTLTCTVSGFSLRNARMAVSWIRQPP | 323 | Heavy chain variable domain |
| GKTLEWLAHIFSNDEKSYSTSLKSRLTISKDTSKSQVVLTMTN | | of anti-FLT3 antibody FL_16 |
| MDPVDTATYYCARIVGYGSGWYGYFDYWGQGTLVTVSS | | |
| DIQMTQSPSSVSASVGDRVTITCRASQDIRYDLAWYQQKPGKA | 324 | Light chain variable domain |
| PKRLIYAASSLQSGVPSRFSGSGSGTEFTLTISSLQPEDFATY | | of anti-FLT3 antibody FL_16 |
| YCLQHNFYPLTFGGGTKVEIK | | |
| EVQLVESGGGLVQPGGSLRLSCAASGFTFSSYGMHWVRQAPGK | 325 | Heavy chain variable domain |
| GLEWVAVISYDGSNKYYADSVKGRFTISRDNSKNTLYLQMNSL | | of anti-FLT3 antibody |
| RAEDTAVYYCANLAPWAAYWGQGTLVTVSS | | m10006 |
| EIVLTQSPLSLPVTPGEPASISCRSSQSLLHSNGYNYLDWYLQ | 326 | Light chain variable domain |
| KPGQSPQLLIYLGSNRASGVPDRFSGSGSGTDFTLKISRVEAE | | of anti-FLT3 antibody |
| DVGVYYCMQALQTPHTFGQGTKLEIK | | m10006 |
| QVQLKQSGPGLVQPSQSLSITCTVSGFSLTNYGLHWVRQSPGK | 327 | Heavy chain variable domain |
| GLEWLGVIWSGGSTDYNAAFISRLSISKDNSKSQVFFKMNSLQ | | of anti-FLT3 antibody BV10 |
| ADDTAIYYCARKGGIYYANHYYAMDYWGQGTSVTVSS | | |
| DIVMTQSPSSLSVSAGEKVTMSCKSSQSLLNSGNQKNYMAWYQ | 328 | Light chain variable domain |
| QKPGQPPKLLIYGASTRESGVPDRFTGSGSGTDFTLTISSVQA | | of anti-FLT3 antibody BV10 |
| EDLAVYYCQNDHSYPLTFGAGTKLELKR | | |
| QVQLVQSGAEVKKPGSSVKVSCKASGYTFTDYNMHWVRQAPGQ | 329 | Heavy chain variable domain |
| GLEWIGYIYPYNGGTGYNQKFKSKATITADESTNTAYMELSSL | | of anti-CD33 antibody |
| RSEDTAVYYCARGRPAMDYWGQGTLVTVSS | | lintuzumab |
| DIQMTQSPSSLSASVGDRVTITCRASESVDNYGISFMNWFQQK | 330 | Light chain variable domain |
| PGKAPKLLIYAASNQGSGVPSRFSGSGSGTDFTLTISSLQPDD | | of anti-CD33 antibody |
| FATYYCQQSKEVPWTFGQGTKVEIK | | lintuzumab |
| EVQLVQSGAEVKKPGSSVKVSCKASGYTITDSNIHWVRQAPGQ | 331 | Heavy chain variable domain |
| SLEWIGYIYPYNGGTDYNQKFKNRATLTVDNPTNTAYMELSSL | | of anti-CD33 antibody |
| RSEDTAFYYCVNGNPWLAYWGQGTLVTVSS | | gemtuzumab |
| DIQLTQSPSTLSASVGDRVTITCRASESLDNYGIRFLTWFQQK | 332 | Light chain variable domain |
| PGKAPKLLMYAASNQGSGVPSRFSGSGSGTEFTLTISSLQPDD | | of anti-CD33 antibody |
| FATYYCQQTKEVPWSFGQGTKVEVKR | | gemtuzumab |

TABLE B

| | SEQ ID | |
|---|---|---|
| Nucleic Acid Sequence | NO: | Description |
| GAAGTGCAACTTGTTCAGAGCGGGGCAGAAGTTAAGAAGCCAGGCGCTTC | 333 | Heavy chain |
| CGTCAAGGTGAGTTGCAAGGCAAGTGGATACACCTTTACGAGTTATTATA | | variable |
| TGCACTGGGCACGGCAGGCCCCTGGTCAGGGCCTCGAATGGATGGGGATT | | domain of |
| ATAAATCCTTCTGGCGGGTCAACCAGCTACGCACAAAAATTTCAAGGTCG | | anti-FLT3 |
| GGTGACAATGACGCGCGACACGTCAACGAGTACAGTGTATATGGAATTGT | | antibody D4- |
| CTAGCCTGAGGTCCGAGGATACTGCTGTCTATTATTGTGCTCGCGTGGTC | | 3 |
| GCTGCTGCTGTGGCAGACTACTGGGGTCAGGGTACACTTGTGACGGTAAG | | |
| CAGC | | |
| GACGTAGTTATGACACAGTCTCCACTGTCATTGCCAGTAACACCAGGTGA | 334 | Light chain |
| GCCCGCCTCCATCTCATGTAGATCCTCCCAATCTCTCCTTCATTCAAACG | | variable |
| GGTATAATTATCTCGACTGGTATTTGCAGAAACCGGGCCAGAGCCCTCAA | | domain of |
| CTGCTCATCTATTTGGGGAGCAACCGGGCCTCTGGTGTCCCTGATAGATT | | anti-FLT3 |
| CTCCGGGAGTGGATCAGGTACGGATTTTACACTGAAGATCAGCAGGGTGG | | antibody D4- |
| AAGCAGAAGATGTTGGTGTGTATTACTGTATGCAATCACTCCAGACCCCG | | 3 |
| TTTACCTTTGGGCCTGGAACAAAGGTAGATATTAAA | | |
| GAGGTTCAACTGGTACAAAGCGGAGCCGAGGTAAAGAAACCAGGGAGTAG | 335 | Heavy chain |
| CGTCAAAGTGTCCTGCAAAGCCTCAGGCGGCACATTCAGTAGCTATGCTA | | variable |
| TTTCATGGGTACGCCAAGCACCAGGACAGGGGCTGGAGTGGATGGGCGGG | | domain of |
| ATTATCCCCATCTTCGGTACGGCAAACTATGCACAAAAGTTCCAGGGACG | | anti-FLT3 |
| AGTCACCATCACGGCTGATAAGTCCACCTCCACCGCCTATATGGAGCTGA | | antibody |
| GTTCCCTTCGGAGCGAGGATACTGCTGTGTATTATTGTGCCACGTTCGCA | | NC7 |
| CTGTTCGGTTTTCGGGAGCAGGCGTTTGATATTTGGGGACAAGGCACAAC | | |
| GGTCACGGTCAGTTCA | | |
| GACATTCAGATGACCCAGAGTCCCTCTTCATTGAGTGCGAGCGTCGGTGA | 336 | Light chain |
| TCGGGTTACGATAACCTGTAGGGCCTCCCAAAGTATATCATCATATTTGA | | variable |
| ACTGGTACCAACAGAAACCTGGGAAAGCGCCGAAGCTCCTTATCTATGCT | | domain of |
| GCCAGCTCTTTGCAAAGCGGTGTGCCCTCACGGTTCTCCGGTAGTGGGTC | | anti-FLT3 |
| CGGGACCGACTTCACTTTGACCATCAGCAGCCTTCAGCCAGAGGATCTTG | | antibody |
| CCACTTATTACTGCCAGCAATCTTATAGCACACCGTTTACATTCGGTCCA | | NC7 |
| GGCACAAAGGTAGACATTAAG | | |
| GAGGTACAGCTTGTGCAGAGTGGAGCAGAAGTTAAAAAACCCGGAGCTTC | 337 | Heavy chain |
| CGTGAAGGTAAGCTGCAAGGCTTCAGGATATACATTTACTAGCTACTACA | | variable |
| TGCACTGGGTCCGCCAAGCTCCGGGCCAAGGCCTTGAATGGATGGGCATC | | domain of |
| ATAAATCCCAGTGGAGGCTCAACGAGCTATGCACAAAAGTTCCAAGGGCG | | anti-FLT3 |
| CGTTACCATGACGCGCGACACCAGCACGTCCACCGTCTATATGGAACTCT | | antibody |
| CAAGTTTGCGATCTGAAGATACGGCTGTCTACTATTGCGCACGAGGGGTC | | EB10 |
| GGAGCGCATGACGCCTTCGACATCTGGGGACAAGGGACTACAGTAACTGT | | |
| GTCAAGC | | |
| GATGTTGTTATGACACAGTCTCCCCTCTCTTTGCCTGTTACGCCTGGCGA | 338 | Light chain |
| GCCCGCCTCTATTTCTTGTCGATCTAGTCAGAGCCTGCTGCATTCTAATG | | variable |
| GAAACAACTATTTGGACTGGTACTTGCAAAAGCCGGGTCAAAGTCCC | | domain of |
| | | anti-FLT3 |
| | | antibody |
| | | EB10 |
| CAAGTCCAACTTCAGCAGCCAGGCGCTGAGTTGGTTAAACCGGGCGCAAG | 339 | Heavy chain |
| CCTCAAACTTAGTTGCAAGTCATCCGGATATACTTTCACGTCTTATTGGA | | variable |
| TGCATTGGGTACGACAAAGACCTGGTCACGGCCTCGAATGGATTGGCGAA | | domain of |
| ATCGACCCGTCAGACAGCTACAAGGATTACAACCAGAAATTCAAAGATAA | | anti-FLT3 |
| GGCAACACTTACTGTGGATCGCTCAAGTAACACGGCTTACATGCACCTCT | | antibody 4G8 |
| CTTCACTCACGTCTGACGACAGTGCGGTGTATTATTGCGCCCGCGCTATT | | |
| ACAACAACCCCTTTCGATTTCTGGGGCCAGGGTACTACGCTCACAGTCTC | | |
| ATCC | | |
| GATATCGTCCTCACCCAATCCCCGGCTACTTTGAGTGTAACACCAGGCGA | 340 | Light chain |
| CAGCGTGTCACTGTCATGCCGAGCCTCCCAGTCAATCAGCAATAATCTGC | | variable |
| ATTGGTATCAACAGAAATCACACGAATCCCCCCGACTTTTGATAAAGTAT | | domain of |
| GCGTCACAGTCCATATCAGGCATTCCCAGTAGGTTTTCAGGCAGTGGTTC | | anti-FLT3 |
| AGGTACTGACTTCACCCTCTCCATTAACTCTGTAGAAACAGAGGACTTTG | | antibody 4G8 |
| GCGTCTACTTCTGTCAGCAATCCAACACCTGGCCTTATACATTCGGCGGC | | |
| GGCACTAAGCTGGAAATTAAGAGA | | |
| CAAGTAACCCTTAAAGAGTCCGGCCCCACTTTGGTTAAACCTACTGAAAC | 341 | Heavy chain |
| ACTTACACTCACATGCACATTGTCCGGCTTTTCACTCAACAACGCAAGGA | | variable |
| TGGGTGTGTCCTGGATTCGCCAGCCCCCTGGAAAATGTTTGGAATGGCTC | | domain of |
| GCTCATATATTTAGCAACGACGAGAAAAGTTACTCAACTTCACTCAAGAA | | anti-FLT3 |
| CCGCCTCACTATTAGCAAAGATTCCTCCAAAACCCAAGTAGTTCTGACAA | | antibody |
| TGACGAATGTAGACCCAGTCGATACTGCAACTTACTATTGCGCACGAATA | | FL39 |
| GTCGGTTACGGGAGTGGCTGGTATGGGTTTTTCGACTATTGGGGACAGGG | | |
| CACTCTTGTAACAGTAAGTAGC | | |
| GACATCCAGATGACTCAATCTCCATCTAGCCTCTCAGCGTCTGTGGGCGA | 342 | Light chain |
| TCGAGTCACCATCACCTGTAGGGCTTCCCAGGGTATAAGGAATGATTTGG | | variable |
| GCTGGTACCAGCAAAAACCGGGTAAGGCTCCGAAACGACTGATATACGCA | | domain of |
| GCTTCTACGTTGCAATCCGGGGTGCCATCCAGATTTAGTGGCAGCGGGAG | | anti-FLT3 |
| CGGTACTGAGTTTACGCTGACTATCTCCTCACTTCAGCCAGAGGATTTCG | | antibody |
| CCACGTACTATTGTCTGCAACACAACTCCTATCCGCTGACCTTCGGGTGC | | FL39 |
| GGGACAAAGGTGGAAATTAAA | | |
| CAAGTGACCTTGAAGGAGTCAGGGCCAGTGTTGGTAAAACCTACTGAGAC | 343 | Heavy chain |
| TCTCACGTTGACATGCACGGTATCAGGTTTCAGCCTGAGGAACGCTCGGA | | variable |
| TGGCCGTCAGTTGGATACGCCAGCCGCCAGGCAAAACTCTTGAATGGTTG | | domain of |
| GCGCACATATTCAGTAACGACGAGAAATCTTACTCTACATCCCTTAAGTC | | anti-FLT3 |
| TCGCCTCACCATTTCTAAAGACACATCCAAATCACAAGTGGTACTCACGA | | antibody |
| TGACAAACATGGACCCTGTTGACACTGCTACATATTATTGTGCTAGGATA | | FL16 |
| GTGGGCTACGGTAGCGGATGGTACGGTTATTTTGATTACTGGGGACAAGG | | |
| GACGCTTGTTACGGTGTCCTCA | | |
| GACATTCAGATGACCCAGTCTCCGTCCAGCGTTAGCGCAAGCGTGGGGGA | 345 | Light chain |
| TAGAGTCACTATTACGTGTAGAGCCAGTCAAGATATACGGTACGATCTTG | | variable |
| CTTGGTATCAGCAAAAACCGGGAAAAGCCCCGAAGAGACTTATATATGCA | | domain of |
| GCTTCCTCCTTGCAAAGCGGGGTCCCATCCCGGTTTAGTGGTAGTGGTTC | | anti-FLT3 |
| CGGAACAGAGTTCACGCTGACTATTTCATCACTGCAACCCGAAGATTTTG | | antibody |
| CCACCTACTACTGCCTTCAACACAATTTCTATCCTCTTACCTTCGGCGGA | | FL16 |
| GGTACTAAGGTAGAGATTAAG | | |
| GAAGTACAGTTGGTTGAGAGTGGTGGAGGACTCGTTCAACCTGGCGGTAG | 346 | Heavy chain |
| TTTGCGACTCAGCTGCGCGGCTTCCGGTTTCACCTTCTCATCCTATGGGA | | variable |
| TGCACTGGGTCAGACAAGCCCCTGGAAAGGGCCTCGAATGGGTTGCTGTG | | domain of |
| ATTAGCTATGACGGCTCTAATAAATACTATGCAGATAGTGTAAAGGGGAG | | anti-FLT3 |
| ATTTACGATTTCTCGCGATAATAGCAAAAATACGCTGTACCTGCAAATGG | | antibody |
| AAACCAACAGCCTGCGAGCGGAAGATACGGCGGTTTATTACTGCGCGAAT | | ml0006 |
| CTTGCCCCGTGGGCAGCATACTGGGGACAGGGGACGTTGGTGACGGTAAG | | |
| CAGT | | |
| GAGATTGTGCTCACCCAGTCTCCACTCAGCCTTCCTGTAACGCCCGGTGA | 347 | Light chain |
| GCCTGCCTCTATATCATGCCGAAGTTCCCAAAGCCTTCTGCACTCAAACG | | variable |
| GCTATAACTACTTGGACTGGTACCTCCAGAAGCCCGGCCAAAGTCCTCAA | | domain of |
| CTGTTGATATACCTGGGGTCCAACCGGGCATCAGGAGTACCTGATAGATT | | anti-FLT3 |
| CTCAGGAAGTGGGTCAGGAACCGACTTCACGCTGAAAATTAGTCGCGTAG | | antibody |
| AGGCGGAAGATGTAGGTGTGTATTACTGTATGCAGGCGTTGCAAACACCG | | m10006 |
| CACACTTTTGGACAGGGAACCAAACTGGAAATAAAGACCAGTAGTGGT | | |
| CAGGTCCAACTGAAACAAAGCGGTCCCGGTCTTGTCCAGCCCTCCCAATC | 348 | Heavy chain |
| TCTCAGTATTACTTGCACTGTGTCAGGTTTCAGCCTCACGAACTACGGTC | | variable |
| TGCATTGGGTCCGCCAGTCTCCAGGAAAAGGCCTGGAGTGGCTCGGTGTT | | domain of |
| ATCTGGAGTGGTGGAAGTACGGATTACAATGCTGCCTTTATCTCTCGGCT | | anti-FLT3 |
| CAGTATCTCCAAAGATAACTCTAAGTCCCAAGTCTTTTTCAAAATGAACT | | antibody |
| CTTTGCAGGCAGATGATACGGCCATATACTATTGCGCACGCAAGGGTGGG | | BV10 |
| ATCTACTATGCAAACCACTATTACGCGATGGACTACTGGGGCCAAGGCAC | | |
| GAGTGTTACCGTGTCAAGC | | |
| GACATAGTGATGACTCAGTCTCCGTCCTCTCTTTCCGTGAGTGCGGGCGA | 349 | Light chain |
| AAAGGTTACCATGTCCTGCAAAAGTTCACAGTCACTTCTCAATTCTGGCA | | variable |
| ACCAAAAAAATTACATGGCATGGTATCAACAGAAACCAGGTCAGCCGCCA | | domain of |
| AAGCTCCTCATATATGGTGCATCAACGCGAGAGTCAGGCGTACCTGACAG | | anti-FLT3 |
| GTTTACCGGATCTGGCAGCGGTACAGACTTTACTCTTACCATATCAAGTG | | antibody |
| TGCAGGCAGAGGACTTGGCGGTATACTATTGTCAAAACGATCATAGTTAC | | BV10 |
| CCTCTTACATTTGGCGCGGGCACTAAACTGGAGCTGAAACGC | | |
| CAGGTTCAGCTGGTTCAGTCTGGCGCCGAAGTGAAGAAACCTGGCAGCAG | 350 | Heavy chain |
| CGTGAAGGTGTCCTGCAAGGCCAGCGGCTACACCTTTACCGACTACAACA | | variable |
| TGCACTGGGTCCGACAGGCCCCTGGACAAGGACTTGAGTGGATCGGCTAC | | domain of |
| ATCTACCCCTACAATGGCGGCACCGGCTACAACCAGAAGTTCAAGAGCAA | | anti-CD33 |
| GGCCACCATCACCGCCGACGAGAGCACAAACACCGCCTACATGGAACTGA | | antibody |
| GCAGCCTGAGAAGCGAGGACACCGCCGTGTACTACTGTGCCAGAGGCAGA | | lintuzumab |
| CCCGCCATGGATTATTGGGGACAGGGCACCCTGGTCACCGTTTCTAGC | | |
| GATATCCAGATGACACAGAGCCCCAGCAGCCTGTCTGCCAGCGTGGGAGA | 351 | Light chain |
| TAGAGTGACCATCACCTGTAGAGCCAGCGAGAGCGTGGACAACTACGGCA | | variable |
| TCAGCTTCATGAACTGGTTCCAGCAGAAGCCCGGCAAGGCCCCTAAGCTG | | domain of |
| CTGATCTACGCCGCCAGCAATCAAGGCAGCGGAGTGCCTAGCAGATTTTC | | anti-CD33 |
| CGGCTCTGGCAGCGGCACCGATTTCACCCTGACAATCTCTAGCCTCCAGC | | antibody |
| CTGACGACTTCGCCACCTACTACTGCCAGCAGAGCAAAGAGGTGCCCTGG | | lintuzumab |
| ACATTCGGCCAGGGCACAAAGGTGGAAATCAAG | | |
| GAAGTGCAGCTGGTTCAGTCTGGCGCCGAAGTGAAGAAACCTGGCAGCAG | 352 | Heavy chain |
| CGTGAAGGTGTCCTGCAAGGCCAGCGGCTACACCATCACCGACAGCAACA | | variable |
| TCCACTGGGTCCGACAGGCTCCAGGCCAGTCTCTTGAGTGGATCGGCTAC | | domain of |
| ATCTACCCCTACAACGGCGGCACCGACTACAACCAGAAGTTCAAGAACCG | | anti-CD33 |
| GGCCACACTGACCGTGGACAACCCTACCAATACCGCCTACATGGAACTGA | | antibody |
| GCAGCCTGCGGAGCGAGGACACCGCCTTTTACTACTGCGTGAACGGCAAC | | gemtuzumab |
| CCCTGGCTGGCCTATTGGGGACAGGGAACACTGGTCACAGTGTCTAGC | | |
| GATATTCAGCTGACACAGAGCCCCAGCACACTGTCTGCCTCTGTGGGCGA | 353 | Light chain |
| CAGAGTGACCATCACCTGTAGAGCCAGCGAGAGCCTGGACAACTACGGCA | | variable |
| TCAGATTTCTGACCTGGTTCCAGCAGAAGCCCGGCAAGGCTCCTAAGCTG | | domain of |
| CTGATGTACGCCGCCAGCAATCAAGGCAGCGGAGTGCCTAGCAGATTTTC | | anti-CD33 |
| CGGCTCTGGCAGCGGCACAGAGTTCACCCTGACAATCTCTAGCCTCCAGC | | antibody |
| CTGACGACTTCGCCACCTACTACTGCCAGCAGACCAAAGAGGTGCCCTGG | | gemtuzumab |
| TCCTTTGGACAGGGCACCAAGGTGGAAGTGAAGCGG | | |

TABLE A2

| | SEQ ID | CDR- | SEQ ID | CDR- | SEQ ID | CDR- | SEQ ID | CDR- | SEQ ID | CDR- | SEQ ID | CDR- |
|---|---|---|---|---|---|---|---|---|---|---|---|---|
| Ab | NO | H1 | NO | H2 | NO | H3 | NO | L1 | NO | L2 | NO | L3 |
| FLT3 | 354 | GYTF | 355 | IINPSG | 356 | VVAAA | 357 | RSSQS | 358 | LGSN | 359 | MQSL |
| D4-3 | | TSYY | | GSTSYA | | VADY | | LLHSN | | RA | | QTPF |
| | | MH | | QKFQG | | | | GYNYL | | | | T |
| | | | | | | | | D | | | | |
| FLT3 | 360 | GGTF | 361 | GIIPIF | 362 | FALFG | 363 | RASQS | 364 | AASS | 365 | QQSY |
| NC7 | | SSYA | | GTANYA | | FREQA | | ISSYL | | LQS | | STPF |
| | | IS | | QKFQG | | FDI | | N | | | | T |
| FLT3 | 366 | GYTF | 367 | IINPSG | 368 | GVGAH | 369 | RSSQS | 370 | LGSN | 371 | MQG |
| EB10 | | TSYY | | GSTSYA | | DAFDI | | LLHSN | | RA | | THPA |
| | | MH | | QKFQG | | | | GNNY | | | | IS |
| | | | | | | | | LD | | | | |
| FLT3 | 372 | SYWM | 373 | EIDPSD | 374 | AITTT | 375 | RASQS | 376 | YASQ | 377 | QQSN |
| 4G8 | | H | | SYKDYN | | PFDF | | ISNNL | | SIS | | TWPY |
| | | | | QKFKD | | | | H | | | | T |
| FLT3 | 378 | NARM | 379 | HIFSND | 380 | IVGYG | 381 | RASQG | 382 | AAST | 383 | LQHN |
| FL_39 | | GVS | | EKSYST | | SGWYG | | IRNDL | | LQS | | SYPL |
| | | | | SLKN | | FFDY | | G | | | | T |
| FLT3 | 384 | NARM | 385 | HIFSND | 386 | IVGYG | 387 | RASQD | 388 | AASS | 389 | LQHN |
| FL_16 | | AVS | | EKSYST | | SGWYG | | IRYDL | | LQS | | FYPL |
| | | | | SLKS | | YFDY | | A | | | | T |
| FLT3 | 390 | GFTF | 391 | ISYDGS | 392 | ANLAP | 393 | QSLLH | 394 | LGS | 395 | MQAL |
| m10006 | | SSYG | | NK | | WAAYW | | SNGYN | | | | QTPH |
| | | | | | | | | Y | | | | T |
| FLT3 | 396 | NYGL | 397 | VIWSGG | 398 | KGGIY | 399 | KSSQS | 400 | GAST | 401 | QNDH |
| BV10 | | H | | STDYNA | | YANHY | | LLNSG | | RES | | SYPL |
| | | | | AFIS | | YAMDY | | NQKNY | | | | T |
| | | | | | | | | MA | | | | |
| CD33 | 402 | DYNM | 403 | YIYPYN | 404 | GRPAM | 405 | RASES | 406 | AASN | 407 | QQSK |
| anti- | | H | | GGTGYN | | DYWGQ | | VDNYG | | QGS | | EVPW |
| body | | | | QKFKSK | | | | ISFMN | | | | T |
| lin- | | | | A | | | | | | | | |
| tuzu- | | | | | | | | | | | | |
| mab | | | | | | | | | | | | |
| CD33 | 408 | GYTI | 409 | IYPYNG | 410 | VNGNP | 411 | ESLDN | 412 | AAS | 413 | QQTK |
| anti- | | TDSN | | GT | | WLAY | | YGIRF | | | | EVPW |
| body | | | | | | | | | | | | S |
| gemtu- | | | | | | | | | | | | |
| zumab | | | | | | | | | | | | |

In some embodiments, an antigen-binding domain specific for CD33 includes a heavy chain variable (VH) region and a light chain variable (VL) region, wherein: the CD33-VH includes: a heavy chain complementarity determining region 1 (CDR-H1) having the amino acid sequence of DYNMH (SEQ ID NO: 402), a heavy chain complementarity determining region 2 (CDR-H2) having the amino acid sequence of YIYPYNGGTGYNQKFKSKA (SEQ ID NO: 403), and a heavy chain complementarity determining region 3 (CDR-H3) having the amino acid sequence of GRPAMDYWGQ (SEQ ID NO: 404), and the CD33-VL includes: a light chain complementarity determining region 1 (CDR-L1) having the amino acid sequence of RASESVDNYGISFMN (SEQ ID NO: 405), a light chain complementarity determining region 2 (CDR-L2) having the amino acid sequence of AASNQGS (SEQ ID NO: 406), and a light chain complementarity determining region 3 (CDR-L3) having the amino acid sequence of QQSKEVPWT (SEQ ID NO: 407), and wherein the amino acid sequences of the CDR-H1, the CDR-H2, the CDR-H3, the CDR-L1, the CDR-L2, and the CDR-L3 of the reference antibody are defined based on the Kabat numbering scheme.

In some embodiments, an antigen-binding domain specific for CD33 includes a CD33-VH having the amino acid sequence QVQLVQSGAEVKKPGSSVKVSCKASGYTFTDYNMHWVRQAPGQGLEWIGYIYPYNGG TGYNQKFKSKATITADESTNTAYMELSSLRSEDTAVYYCARGRPAMDYWGQGTLVTV SS (SEQ ID NO: 329), and a CD33-VL having the amino acid sequence DIQMTQSPSSLSASVGDRVTITCRASESVDNYGISFMNWFQQKPGKAPKLLIYAASNQG SGVPSRFSGSGSGTDFTLTISSLQPDDFATYYCQQSKEVPWTFGQGTKVEIK (SEQ ID NO: 330).

In some embodiments, an antigen-binding domain specific for FLT3 includes a heavy chain variable (VH) region and a light chain variable (VL) region, wherein: the FLT3-VH includes a heavy chain complementarity determining region 1 (CDR-H1) having the amino acid sequence of GGTFSSYAIS (SEQ ID NO: 360), a heavy chain complementarity determining region 2 (CDR-H2) having the amino acid sequence of GIIPIFGTANYAQKFQG (SEQ ID NO: 361), and a heavy chain complementarity determining region 3 (CDR-H3) having the amino acid sequence of FALFGFREQAFDI (SEQ ID NO: 362), and the FLT3-VL includes a light chain complementarity determining region 1 (CDR-L1) having the amino acid sequence of RASQSISSYLN (SEQ ID NO: 363), a light chain complementarity determining region 2 (CDR-L2) having the amino acid sequence of AASSLQS (SEQ ID NO: 364), and a light chain complementarity determining region 3 (CDR-L3) having the amino acid sequence of QQSYSTPFT (SEQ ID NO: 365), and wherein the amino acid sequences of the CDR-H1, the CDR-H2, the CDR-H3, the CDR-L1, the CDR-L2, and the CDR-L3 of the reference antibody are defined based on the Kabat numbering scheme.

In some embodiments, an antigen-binding domain specific for FLT3 includes a FLT3-VH having the amino acid sequence EVQLVQSGAEVKKPGSSVKVSCKASGGTFSSYAISWVRQAPGQGLEWMGGIIPIFGTAN YAQKFQGRVTITADKSTSTAYMELSSLRSEDTAVYYCATFALFGFREQAFDIWGQGTTV TVSS (SEQ ID NO: 315), and a FLT3-VL having the amino acid sequence DIQMTQSPSSLSASVGDRVTITCRASQSISSYLNWYQQKPGKAPKLLIYAASSLQSGVPS RFSGSGSGTDFTLTISSLQPEDLATYYCQQSYSTPFTFGPGTKVDIK (SEQ ID NO: 316).

In some embodiments, a bivalent-bispecific aCAR includes an antigen-binding domain specific for FLT3 and an antigen-binding domain specific for CD33.

In some embodiments, a bivalent-bispecific aCAR specific for FLT3 and CD33 includes the amino acid sequence

(SEQ ID NO: 414)
MLLLVTSLLLCELPHPAFLLIPEVQLVQSGAEVKKPGSSVKVSCKASGGT
FSSYAISWVRQAPGQGLEWMGGIIPIFGTANYAQKFQGRVTITADKSTST
AYMELSSLRSEDTAVYYCATFALFGFREQAFDIWGQGTTVTVSSGGGGSQ
VQLVQSGAEVKKPGSSVKVSCKASGYTFTDYNMHWVRQAPGQGLEWIGYI
YPYNGGTGYNQKFKSKATITADESTNTAYMELSSLRSEDTAVYYCARGRP
AMDYWGQGTLVTVSSGSTSGSGKPGSGEGSTKGDIQMTQSPSSLSASVGD
RVTITCRASESVDNYGISFMNWFQQKPGKAPKLLIYAASNQGSGVPSRFS
GSGSGTDFTLTISSLQPDDFATYYCQQSKEVPWTFGQGTKVEIKGGGGSD
IQMTQSPSSLSASVGDRVTITCRASQSISSYLNWYQQKPGKAPKLLIYAA
SSLQSGVPSRFSGSGSGTDFTLTISSLOPEDLATYYCQQSYSTPFTFGPG
TKVDIKALSNSIMYFSHFVPVFLPAKPTTTPAPRPPTPAPTIASQPLSLR
PEACRPAAGGAVHTRGLDFACDIYIWAPLAGTCGVLLLSLVITLYCNHRR
SKRSRLLHSDYMNMTPRRPGPTRKHYQPYAPPRDFAAYRSRVKFSRSADA
PAYKQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR.

In some embodiments, a bivalent-bispecific aCAR specific for FLT3 and CD33 includes the amino acid sequence

(SEQ ID NO: 415)
MLLLVTSLLLCELPHPAFLLIPEVQLVQSGAEVKKPGSSVKVSCKASGGT
FSSYAISWVRQAPGQGLEWMGGIIPIFGTANYAQKFQGRVTITADKSTST
AYMELSSLRSEDTAVYYCATFALFGFREQAFDIWGQGTTVTVSSGGGGSG
GGGSQVQLVQSGAEVKKPGSSVKVSCKASGYTFTDYNMHWVRQAPGQGLE
WIGYIYPYNGGTGYNQKFKSKATITADESTNTAYMELSSLRSEDTAVYYC
ARGRPAMDYWGQGTLVTVSSGGGGSGGGGSDIQMTQSPSSLSASVGDRVT
ITCRASESVDNYGISFMNWFQQKPGKAPKLLIYAASNQGSGVPSRFSGSG
SGTDFTLTISSLQPDDFATYYCQQSKEVPWTFGQGTKVEIKGGGGSGGGG
SDIQMTQSPSSLSASVGDRVTITCRASQSISSYLNWYQQKPGKAPKLLIY
AASSLQSGVPSRFSGSGSGTDFTLTISSLQPEDLATYYCQQSYSTPFTFG
PGTKVDIKALSNSIMYFSHFVPVFLPAKPTTTPAPRPPTPAPTIASQPLS
LRPEACRPAAGGAVHTRGLDFACDIYIWAPLAGTCGVLLLSLVITLYCNH
RRSKRSRLLHSDYMNMTPRRPGPTRKHYQPYAPPRDFAAYRSRVKFSRSA
DAPAYKQGQNQLYNELNLGRREEYDVLDKRRGRDPEMGGKPRRKNPQEGL
YNELQKDKMAEAYSEIGMKGERRRGKGHDGLYQGLSTATKDTYDALHMQA
LPPR.

### Engineered Nucleic Acids

Provided herein are multicistronic expression systems that include engineered nucleic acids encoding (i) a membrane-cleavable chimeric protein; (ii) a bivalent aCAR; and (iii) an iCAR.

In certain embodiments described herein, the multicistronic expression systems included engineered nucleic acids that encode an expression cassette containing a promoter and an exogenous polynucleotide sequence encoding each of (i) a membrane-cleavable chimeric protein; (ii) a bivalent aCAR; and (iii) an iCAR.

The promoter is operably linked to expression cassette of the multicistronic system such that the exogenous polynucleotide sequence encoding each of the membrane-cleavable chimeric protein, the bivalent aCAR, and the iCAR is configured to be expressed as a single polypeptide.

An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a murine nucleotide sequence, a bacterial nucleotide sequence, a human nucleotide sequence, and/or a viral nucleotide sequence. The term “engineered nucleic acids” includes recombinant nucleic acids and synthetic nucleic acids. A “recombinant nucleic acid” refers to a molecule that is constructed by joining nucleic acid molecules and, in some embodiments, can replicate in a live cell. A “synthetic nucleic acid” refers to a molecule that is amplified or chemically, or by other means, synthesized. Synthetic nucleic acids include those that are chemically modified, or otherwise modified, but can base pair with naturally-occurring nucleic acid molecules. Modifications include, but are not limited to, one or more modified internucleotide linkages and non-natural nucleic acids. Modifications are described in further detail in U.S. Pat. No. 6,673,611 and U.S. Application Publication 2004/0019001 and, each of which is incorporated by reference in their entirety. Modified internucleotide linkages can be a phosphorodithioate or phosphorothioate linkage. Non-natural nucleic acids can be a locked nucleic acid (LNA), a peptide nucleic acid (PNA), glycol nucleic acid (GNA), a phosphorodiamidate morpholino oligomer (PMO or “morpholino”), and threose nucleic acid (TNA). Non-natural nucleic acids are described in further detail in International Application WO 1998/039352, U.S. Application Pub. No. 2013/0156849, and U.S. Pat. Nos. 6,670,461; 5,539,082; 5,185,444, each herein incorporated by reference in their entirety. Recombinant nucleic acids and synthetic nucleic acids also include those molecules that result from the replication of either of the foregoing. Engineered nucleic acid of the present disclosure may be encoded by a single molecule (e.g., included in the same plasmid or other vector) or by multiple different molecules (e.g., multiple different independently-replicating molecules). Engineered nucleic acids can be an isolated nucleic acid. Isolated nucleic acids include, but are not limited to a cDNA polynucleotide, an RNA polynucleotide, an RNAi oligonucleotide (e.g., siRNAs, miRNAs, antisense oligonucleotides, shRNAs, etc.), an mRNA polynucleotide, a circular plasmid, a linear DNA fragment, a vector, a minicircle, a ssDNA, a bacterial artificial chromosome (BAC), and yeast artificial chromosome (YAC), and an oligonucleotide.

Engineered nucleic acid of the present disclosure may be produced using standard molecular biology methods (see, e.g., Green and Sambrook, Molecular Cloning, A Laboratory Manual, 2012, Cold Spring Harbor Press). In some embodiments, engineered nucleic acid constructs are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the ′Y extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies. In some embodiments, engineered nucleic acid constructs are produced using IN-FUSION® cloning (Clontech).

### Promoters

In general, in all embodiments described herein, the engineered nucleic acids encoding the membrane-cleavable chimeric protein, the bivalent aCAR, and the iCAR encode an expression cassette containing a promoter. In some embodiments, an engineered nucleic acid (e.g., an engineered nucleic acid comprising an expression cassette) comprises a promoter operably linked to a nucleotide sequence (e.g., an exogenous polynucleotide sequence) encoding at least 2 distinct proteins. For example, the engineered nucleic acid may comprise a promoter operably linked to a nucleotide sequence encoding at least 3, at least 4, at least 5, at least 6, at least 7, at least 8, at least 8, at least 9, or at least 10 distinct proteins. In some embodiments, an engineered nucleic acid comprises a promoter operably linked to a nucleotide sequence encoding 1, 2, 3, 4, 5, 6, 7, 8, 9, 10, or more distinct proteins. In some embodiments, an engineered nucleic acid (e.g., an engineered nucleic acid comprising an expression cassette) comprises a promoter operably linked to a nucleotide sequence (e.g., an exogenous polynucleotide sequence) encoding at least 2 membrane-cleavable chimeric proteins. For example, the engineered nucleic acid may comprise a promoter operably linked to a nucleotide sequence encoding at least 3, at least 4, at least 5, at least 6, at least 7, at least 8, at least 8, at least 9, or at least 10 membrane-cleavable chimeric proteins. In some embodiments, an engineered nucleic acid comprises a promoter operably linked to a nucleotide sequence encoding 1, 2, 3, 4, 5, 6, 7, 8, 9, 10, or more membrane-cleavable chimeric proteins.

A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. Nos. 4,683,202 and 5,928,906).

Promoters of an engineered nucleic acid may be “inducible promoters,” which refer to promoters that are characterized by regulating (e.g., initiating or activating) transcriptional activity when in the presence of, influenced by or contacted by a signal. The signal may be endogenous or a normally exogenous condition (e.g., light), compound (e.g., chemical or non-chemical compound) or protein (e.g., cytokine) that contacts an inducible promoter in such a way as to be active in regulating transcriptional activity from the inducible promoter. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

A promoter is “responsive to” or “modulated by” a local tumor state (e.g., inflammation or hypoxia) or signal if in the presence of that state or signal, transcription from the promoter is activated, deactivated, increased, or decreased. In some embodiments, the promoter comprises a response element. A “response element” is a short sequence of DNA within a promoter region that binds specific molecules (e.g., transcription factors) that modulate (regulate) gene expression from the promoter. Response elements that may be used in accordance with the present disclosure include, without limitation, a phloretin-adjustable control element (PEACE), a zinc-finger DNA-binding domain (DBD), an interferon-gamma-activated sequence (GAS) (Decker, T. et al. J Interferon Cytokine Res. 1997 March; 17 (3): 121-34, incorporated herein by reference), an interferon-stimulated response element (ISRE) (Han, K. J. et al. J Biol Chem. 2004 Apr. 9; 279 (15): 15652-61, incorporated herein by reference), a NF-kappaB response element (Wang, V. et al. Cell Reports. 2012; 2 (4): 824-839, incorporated herein by reference), and a STAT3 response element (Zhang, D. et al. J of Biol Chem. 1996; 271:9503-9509, incorporated herein by reference). Other response elements are encompassed herein. Response elements can also contain tandem repeats (e.g., consecutive repeats of the same nucleotide sequence encoding the response element) to generally increase sensitivity of the response element to its cognate binding molecule. Tandem repeats can be labeled 2X, 3X, 4X, 5X, etc. to denote the number of repeats present.

Non-limiting examples of responsive promoters (also referred to as “inducible promoters”) (e.g., TGF-beta responsive promoters) are listed in Table 5A, which shows the design of the promoter and transcription factor, as well as the effect of the inducer molecule towards the transcription factor (TF) and transgene transcription (T) is shown (B, binding; D, dissociation; n.d., not determined) (A, activation; DA, deactivation; DR, derepression) (see Horner, M. & Weber, W. FEBS Letters 586 (2012) 20784-2096m, and references cited therein). Non-limiting examples of components of inducible promoters include those presented in Table 5B.

TABLE 5A
Examples of Responsive Promoters

| | | | | Response to | |
|---|---|---|---|---|---|
| | Promoter and | Transcription | | inducer | |
| System | operator | factor (TF) | Inducer molecule | TF | T |
| Transcriptional activator-responsive promoters | | | | | |
| AIR | PAIR (OalcA- | AlcR | Acetaldehyde | n.d. | A |
| | PhCMVmin) | | | | |
| ART | PART (OARG- | ArgR-VP16 | 1-Arginine | B | A |
| | PhCMVmin) | | | | |
| BIT | PBIT3 (OBirA3- | BIT (BirA- | Biotin | B | A |
| | PhCMVmin) | VP16) | | | |
| Cumate-activator | PCR5 (OCu06- | cTA (CymR- | Cumate | D | DA |
| | PhCMVmin) | VP16) | | | |
| Cumate-reverse | PCR5 (OCu06- | rcTA (rCymR- | Cumate | B | A |
| activator | PhCMVmin) | VP16) | | | |
| E-OFF | PETR (OETR- | ET (E-VP16) | Erythromycin | D | DA |
| | PhCMVmin) | | | | |
| NICE-OFF | PNIC (ONIC- | NT (HdnoR- | 6-Hydroxy-nicotine | D | DA |
| | PhCMVmin) | VP16) | | | |
| PEACE | PTtgR1 (OTtgR- | TtgA1 (TtgR- | Phloretin | D | DA |
| | PhCMVmin) | VP16) | | | |
| PIP-OFF | PPIR (OPIR- | PIT (PIP-VP16) | Pristinamycin I | D | DA |
| | Phsp70min) | | | | |
| QuoRex | PSCA (OscbR- | SCA (ScbR- | SCB1 | D | DA |
| | PhCMVmin)PSPA | VP16) | | | |
| | (OpapRI- | | | | |
| | PhCMVmin) | | | | |
| Redox | PROP (OROP- | REDOX (REX- | NADH | D | DA |
| | PhCMVmin) | VP16) | | | |
| TET-OFF | PhCMV*-1 | tTA (TetR- | Tetracycline | D | DA |
| | (OtetO7- | VP16) | | | |
| | PhCMVmin) | | | | |
| TET-ON | PhCMV*-1 | rtTA (rTetR- | Doxycycline | B | A |
| | (OtetO7- | VP16) | | | |
| | PhCMVmin) | | | | |
| TIGR | PCTA (OrheO- | CTA (RheA- | Heat | D | DA |
| | PhCMVmin) | VP16) | | | |
| TraR | O7x(tra box)- | p65-TraR | 3-Oxo-C8-HSL | B | A |
| | PhCMVmin | | | | |
| VAC-OFF | P1VanO2 | VanA1 (VanR- | Vanillic acid | D | DA |
| | (OVanO2- | VP16) | | | |
| | PhCMVmin) | | | | |
| Transcriptional repressor-responsive promoters | | | | | |
| Cumate-repressor | PCuO (PCMV5- | CymR | Cumate | D | DR |
| | OCuO) | | | | |
| E-ON | PETRON8 | E-KRAB | Erythromycin | D | DR |
| | (PSV40-OETR8) | | | | |
| NICE-ON | PNIC (PSV40- | NS (HdnoR- | 6-Hydroxy-nicotine | D | DR |
| | ONIC8) | KRAB) | | | |
| PIP-ON | PPIRON (PSV40- | PIT3 (PIP- | Pristinamycin I | D | DR |
| | OPIR3) | KRAB) | | | |
| Q-ON | PSCAON8 | SCS (ScbR- | SCB1 | D | DR |
| | (PSV40-OscbR8) | KRAB) | | | |
| TET-ON repressor- | OtetO-PHPRT | tTS-H4 (TetR- | Doxycycline | D | DR |
| based | | HDAC4) | | | |
| T-REX | PTetO (PhCMV- | TetR | Tetracycline | D | DR |
| | OtetO2) | | | | |
| UREX | PUREX8 (PSV40- | mUTS (KRAB- | Uric acid | D | DR |
| | OhucO8) | HucR) | | | |
| VAC-ON | PVanON8 | VanA4 (VanR- | Vanillic acid | D | DR |
| | (PhCMV-OVan08) | KRAB) | | | |
| Hybrid promoters | | | | | |
| QuoRexPIP- | OscbR8-OPIR3- | SCAPIT3 | SCB1Pristinamycin I | DD | DADR |
| ON(NOT IF gate) | PhCMVmin | | | | |
| QuoRexE- | OscbR-OETR8- | SCAE-KRAB | SCB1Erythromycin | DD | DADR |
| ON(NOT IF gate) | PhCMVmin | | | | |
| TET-OFFE- | OtetO7-OETR8- | tTAE-KRAB | TetracyclineErythromycin | DD | DADR |
| ON(NOT IF gate) | PhCMVmin | | | | |
| TET-OFFPIP- | Otet07-OPIR3- | tTAPIT3E- | TetracyclinePristinamycin | DDD | DADRDR |
| ONE-ON | OETR8- | KRAB | IErythromycin | | |
| | PhCMVmin | | | | |

TABLE 5B
Exemplary Components of Inducible Promoters

| Name | DNA SEQUENCE |
|---|---|
| minimal promoter; minP | AGAGGGTATATAATGGAAGCTCGACTTCCAG (SEQ ID NO: 1) |
| NFkB response element | GGGAATTTCCGGGGACTTTCCGGGAATTTCCGGGGACTTTCCGGGAATT |
| protein promoter; 5x | TCC (SEQ ID NO: 2) |
| NFkB-RE | |
| CREB response element | CACCAGACAGTGACGTCAGCTGCCAGATCCCATGGCCGTCATACTGTGA |
| protein promoter; 4x | CGTCTTTCAGACACCCCATTGACGTCAATGGGAGAA (SEQ ID NO: |
| CRE | 3) |
| NFAT response element | GGAGGAAAAACTGTTTCATACAGAAGGCGTGGAGGAAAAACTGTTTCAT |
| protein promoter; 3x | ACAGAAGGCGTGGAGGAAAAACTGTTTCATACAGAAGGCGT (SEQ ID |
| NFAT binding sites | NO: 4) |
| SRF response element | AGGATGTCCATATTAGGACATCTAGGATGTCCATATTAGGACATCTAGG |
| protein promoter; 5x | ATGTCCATATTAGGACATCTAGGATGTCCATATTAGGACATCTAGGATG |
| SRE | TCCATATTAGGACATCT (SEQ ID NO: 5) |
| SRF response element | AGTATGTCCATATTAGGACATCTACCATGTCCATATTAGGACATCTACT |
| protein promoter 2; 5x | ATGTCCATATTAGGACATCTTGTATGTCCATATTAGGACATCTAAAATG |
| SRF-RE | TCCATATTAGGACATCT (SEQ ID NO: 6) |
| AP1 response element | TGAGTCAGTGACTCAGTGAGTCAGTGACTCAGTGAGTCAGTGACTCAG |
| protein promoter; 6x | (SEQ ID NO: 7) |
| AP1-RE | |
| TCF-LEF response | AGATCAAAGGGTTTAAGATCAAAGGGCTTAAGATCAAAGGGTATAAGAT |
| element promoter; 8x | CAAAGGGCCTAAGATCAAAGGGACTAAGATCAAAGGGTTTAAGATCAAA |
| TCF-LEF-RE | GGGCTTAAGATCAAAGGGCCTA (SEQ ID NO: 8) |
| SBEx4 | GTCTAGACGTCTAGACGTCTAGACGTCTAGAC (SEQ ID NO: 9) |
| SMAD2/3 - CAGACA x4 | CAGACACAGACACAGACACAGACA (SEQ ID NO: 10) |
| STAT3 binding site | Ggatccggtactcgagatctgcgatctaagtaagcttggcattccggta |
| | ctgttggtaaagccac (SEQ ID NO: 11) |
| minCMV | taggcgtgtacggtgggaggcctatataagcagagctcgtttagtgaac |
| | cgtcagatcgcctgga (SEQ ID NO: 170) |
| YB_TATA | TCTAGAGGGTATATAATGGGGGCCA (SEQ ID NO: 171) |
| minTK | Ttcgcatattaaggtgacgcgtgtggcctcgaacaccgagcgaccctgc |
| | agcgacccgcttaa (SEQ ID NO: 172) |

Non-limiting examples of promoters include the cytomegalovirus (CMV) promoter, the elongation factor 1-alpha (EF1a) promoter, the elongation factor (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, and the ubiquitin C (UbC) promoter (see Table 5C).

TABLE 5C
Exemplary Constitutive Promoters

| Name | DNA SEQUENCE |
|---|---|
| CMV | GTTGACATTGATTATTGACTAGTTATTAATAGTAATCAATTACGGGGTCATTAGTTCAT |
| | AGCCCATATATGGAGTTCCGCGTTACATAACTTACGGTAAATGGCCCGCCTGGCTGACC |
| | GCCCAACGACCCCCGCCCATTGACGTCAATAATGACGTATGTTCCCATAGTAACGCCAA |
| | TAGGGACTTTCCATTGACGTCAATGGGTGGAGTATTTACGGTAAACTGCCCACTTGGCA |
| | GTACATCAAGTGTATCATATGCCAAGTACGCCCCCTATTGACGTCAATGACGGTAAATG |
| | GCCCGCCTGGCATTATGCCCAGTACATGACCTTATGGGACTTTCCTACTTGGCAGTACA |
| | TCTACGTATTAGTCATCGCTATTACCATGGTGATGCGGTTTTGGCAGTACATCAATGGG |
| | CGTGGATAGCGGTTTGACTCACGGGGATTTCCAAGTCTCCACCCCATTGACGTCAATGG |
| | GAGTTTGTTTTGGCACCAAAATCAACGGGACTTTCCAAAATGTCGTAACAACTCCGCCC |
| | CATTGACGCAAATGGGCGGTAGGCGTGTACGGTGGGAGGTCTATATAAGCAGAGCTC |
| | (SEQ ID NO: 12) |
| EF1a | GGCTCCGGTGCCCGTCAGTGGGCAGAGCGCACATCGCCCACAGTCCCCGAGAAGTTGGG |
| | GGGAGGGGTCGGCAATTGAACCGGTGCCTAGAGAAGGTGGCGCGGGGTAAACTGGGAAA |
| | GTGATGCCGTGTACTGGCTCCGCCTTTTTCCCGAGGGTGGGGGAGAACCGTATATAAGT |
| | GCAGTAGTCGCCGTGAACGTTCTTTTTCGCAACGGGTTTGCCGCCAGAACACAGGTAAG |
| | TGCCGTGTGTGGTTCCCGCGGGCCTGGCCTCTTTACGGGTTATGGCCCTTGCGTGCCTT |
| | GAATTACTTCCACCTGGCTGCAGTACGTGATTCTTGATCCCGAGCTTCGGGTTGGAAGT |
| | GGGTGGGAGAGTTCGAGGCCTTGCGCTTAAGGAGCCCCTTCGCCTCGTGCTTGAGTTGA |
| | GGCCTGGCCTGGGCGCTGGGGCCGCCGCGTGCGAATCTGGTGGCACCTTCGCGCCTGTC |
| | TCGCTGCTTTCGATAAGTCTCTAGCCATTTAAAATTTTTGATGACCTGCTGCGACGCTT |
| | TTTTTCTGGCAAGATAGTCTTGTAAATGCGGGCCAAGATCTGCACACTGGTATTTCGGT |
| | TTTTGGGGCCGCGGGCGGCGACGGGGCCCGTGCGTCCCAGCGCACATGTTCGGCGAGGC |
| | GGGGCCTGCGAGCGCGACCACCGAGAATCGGACGGGGGTAGTCTCAAGCTGGCCGGCCT |
| | GCTCTGGTGCCTGTCCTCGCGCCGCCGTGTATCGCCCCGCCCCGGGCGGCAAGGCTGGC |
| | CCGGTCGGCACCAGTTGCGTGAGCGGAAAGATGGCCGCTTCCCGGTCCTGCTGCAGGGA |
| | GCTCAAAATGGAGGACGCGGCGCTCGGGAGAGCGGGCGGGTGAGTCACCCACACAAAGG |
| | AAAAGGGCCTTTCCGTCCTCAGCCGTCGCTTCATGTGACTCCACGGAGTACCGGGCGCC |
| | GTCCAGGCACCTCGATTAGTTCTCGAGCTTTTGGAGTACGTCGTCTTTAGGTTGGGGGG |
| | AGGGGTTTTATGCGATGGAGTTTCCCCACACTGAGTGGGTGGAGACTGAAGTTAGGCCA |
| | GCTTGGCACTTGATGTAATTCTCCTTGGAATTTGCCCTTTTTGAGTTTGGATCTTGGTT |
| | CATTCTCAAGCCTCAGACAGTGGTTCAAAGTTTTTTTCTTCCATTTCAGGTGTCGTGA |
| | (SEQ ID NO: 13) |
| EFS | GGATCTGCGATCGCTCCGGTGCCCGTCAGTGGGCAGAGCGCACATCGCCCACAGTCCCC |
| | GAGAAGTTGGGGGGAGGGGTCGGCAATTGAACCGGTGCCTAGAGAAGGTGGCGCGGGGT |
| | AAACTGGGAAAGTGATGTCGTGTACTGGCTCCGCCTTTTTCCCGAGGGTGGGGGAGAAC |
| | CGTATATAAGTGCAGTAGTCGCCGTGAACGTTCTTTTTCGCAACGGGTTTGCCGCCAGA |
| | ACACAGCTGAAGCTTCGAGGGGCTCGCATCTCTCCTTCACGCGCCCGCCGCCCTACCTG |
| | AGGCCGCCATCCACGCCGGTTGAGTCGCGTTCTGCCGCCTCCCGCCTGTGGTGCCTCCT |
| | GAACTGCGTCCGCCGTCTAGGTAAGTTTAAAGCTCAGGTCGAGACCGGGCCTTTGTCCG |
| | GCGCTCCCTTGGAGCCTACCTAGACTCAGCCGGCTCTCCACGCTTTGCCTGACCCTGCT |
| | TGCTCAACTCTACGTCTTTGTTTCGTTTTCTGTTCTGCGCCGTTACAGATCCAAGCTGT |
| | GACCGGCGCCTAC (SEQ ID NO: 14) |
| MND | TTTATTTAGTCTCCAGAAAAAGGGGGGAATGAAAGACCCCACCTGTAGGTTTGGCAAGC |
| | TAGGATCAAGGTTAGGAACAGAGAGACAGCAGAATATGGGCCAAACAGGATATCTGTGG |
| | TAAGCAGTTCCTGCCCCGGCTCAGGGCCAAGAACAGTTGGAACAGCAGAATATGGGCCA |
| | AACAGGATATCTGTGGTAAGCAGTTCCTGCCCCGGCTCAGGGCCAAGAACAGATGGTCC |
| | CCAGATGCGGTCCCGCCCTCAGCAGTTTCTAGAGAACCATCAGATGTTTCCAGGGTGCC |
| | CCAAGGACCTGAAATGACCCTGTGCCTTATTTGAACTAACCAATCAGTTCGCTTCTCGC |
| | TTCTGTTCGCGCGCTTCTGCTCCCCGAGCTCAATAAAAGAGCCCA (SEQ ID NO: |
| | 15) |
| PGK | GGGGTTGGGGTTGCGCCTTTTCCAAGGCAGCCCTGGGTTTGCGCAGGGACGCGGCTGCT |
| | CTGGGCGTGGTTCCGGGAAACGCAGCGGCGCCGACCCTGGGTCTCGCACATTCTTCACG |
| | TCCGTTCGCAGCGTCACCCGGATCTTCGCCGCTACCCTTGTGGGCCCCCCGGCGACGCT |
| | TCCTGCTCCGCCCCTAAGTCGGGAAGGTTCCTTGCGGTTCGCGGCGTGCCGGACGTGAC |
| | AAACGGAAGCCGCACGTCTCACTAGTACCCTCGCAGACGGACAGCGCCAGGGAGCAATG |
| | GCAGCGCGCCGACCGCGATGGGCTGTGGCCAATAGCGGCTGCTCAGCGGGGCGCGCCGA |
| | GAGCAGCGGCCGGGAAGGGGCGGTGCGGGAGGCGGGGTGTGGGGCGGTAGTGTGGGCCC |
| | TGTTCCTGCCCGCGCGGTGTTCCGCATTCTGCAAGCCTCCGGAGCGCACGTCGGCAGTC |
| | GGCTCCCTCGTTGACCGAATCACCGACCTCTCTCCCCAG (SEQ ID NO: 16) |
| SFFV | GTAACGCCATTTTGCAAGGCATGGAAAAATACCAAACCAAGAATAGAGAAGTTCAGATC |
| | AAGGGCGGGTACATGAAAATAGCTAACGTTGGGCCAAACAGGATATCTGCGGTGAGCAG |
| | TTTCGGCCCCGGCCCGGGGCCAAGAACAGATGGTCACCGCAGTTTCGGCCCCGGCCCGA |
| | GGCCAAGAACAGATGGTCCCCAGATATGGCCCAACCCTCAGCAGTTTCTTAAGACCCAT |
| | CAGATGTTTCCAGGCTCCCCCAAGGACCTGAAATGACCCTGCGCCTTATTTGAATTAAC |
| | CAATCAGCCTGCTTCTCGCTTCTGTTCGCGCGCTTCTGCTTCCCGAGCTCTATAAAAGA |
| | GCTCACAACCCCTCACTCGGCGCGCCAGTCCTCCGACAGACTGAGTCGCCCGGG (SEQ |
| | ID NO: 17) |
| SV40 | CTGTGGAATGTGTGTCAGTTAGGGTGTGGAAAGTCCCCAGGCTCCCCAGCAGGCAGAAG |
| | TATGCAAAGCATGCATCTCAATTAGTCAGCAACCAGGTGTGGAAAGTCCCCAGGCTCCC |
| | CAGCAGGCAGAAGTATGCAAAGCATGCATCTCAATTAGTCAGCAACCATAGTCCCGCCC |
| | CTAACTCCGCCCATCCCGCCCCTAACTCCGCCCAGTTCCGCCCATTCTCCGCCCCATGG |
| | CTGACTAATTTTTTTTATTTATGCAGAGGCCGAGGCCGCCTCTGCCTCTGAGCTATTCC |
| | AGAAGTAGTGAGGAGGCTTTTTTGGAGGCCTAGGCTTTTGCAAAAAGCT (SEQ ID |
| | NO: 18) |
| UbC | GCGCCGGGTTTTGGCGCCTCCCGCGGGCGCCCCCCTCCTCACGGCGAGCGCTGCCACGT |
| | CAGACGAAGGGCGCAGGAGCGTTCCTGATCCTTCCGCCCGGACGCTCAGGACAGCGGCC |
| | CGCTGCTCATAAGACTCGGCCTTAGAACCCCAGTATCAGCAGAAGGACATTTTAGGACG |
| | GGACTTGGGTGACTCTAGGGCACTGGTTTTCTTTCCAGAGAGCGGAACAGGCGAGGAAA |
| | AGTAGTCCCTTCTCGGCGATTCTGCGGAGGGATCTCCGTGGGGCGGTGAACGCCGATGA |
| | TTATATAAGGACGCGCCGGGTGTGGCACAGCTAGTTCCGTCGCAGCCGGGATTTGGGTC |
| | GCGGTTCTTGTTTGTGGATCGCTGTGATCGTCACTTGGTGAGTTGCGGGCTGCTGGGCT |
| | GGCCGGGGCTTTCGTGGCCGCCGGGCCGCTCGGTGGGACGGAAGCGTGTGGAGAGACCG |
| | CCAAGGGCTGTAGTCTGGGTCCGCGAGCAAGGTTGCCCTGAACTGGGGGTTGGGGGGAG |
| | CGCACAAAATGGCGGCTGTTCCCGAGTCTTGAATGGAAGACGCTTGTAAGGCGGGCTGT |
| | GAGGTCGTTGAAACAAGGTGGGGGGCATGGTGGGCGGCAAGAACCCAAGGTCTTGAGGC |
| | CTTCGCTAATGCGGGAAAGCTCTTATTCGGGTGAGATGGGCTGGGGCACCATCTGGGGA |
| | CCCTGACGTGAAGTTTGTCACTGACTGGAGAACTCGGGTTTGTCGTCTGGTTGCGGGGG |
| | CGGCAGTTATGCGGTGCCGTTGGGCAGTGCACCCGTACCTTTGGGAGCGCGCGCCTCGT |
| | CGTGTCGTGACGTCACCCGTTCTGTTGGCTTATAATGCAGGGTGGGGCCACCTGCCGGT |
| | AGGTGTGCGGTAGGCTTTTCTCCGTCGCAGGACGCAGGGTTCGGGCCTAGGGTAGGCTC |
| | TCCTGAATCGACAGGCGCCGGACCTCTGGTGAGGGGAGGGATAAGTGAGGCGTCAGTTT |
| | CTTTGGTCGGTTTTATGTACCTATCTTCTTAAGTAGCTGAAGCTCCGGTTTTGAACTAT |
| | GCGCTCGGGGTTGGCGAGTGTGTTTTGTGAAGTTTTTTAGGCACCTTTTGAAATGTAAT |
| | CATTTGGGTCAATATGTAATTTTCAGTGTTAGACTAGTAAAGCTTCTGCAGGTCGACTC |
| | TAGAAAATTGTCCGCTAAATTCTGGCCGTTTTTGGCTTTTTTGTTAGAC (SEQ ID |
| | NO: 19) |
| hEF1aV1 | GGCTCCGGTGCCCGTCAGTGGGCAGAGCGCACATCGCCCACAGTCCCCGAGAAGTTGGG |
| | GGGAGGGGTCGGCAATTGAACCGGTGCCTAGAGAAGGTGGCGCGGGGTAAACTGGGAAA |
| | GTGATGTCGTGTACTGGCTCCGCCTTTTTCCCGAGGGTGGGGGAGAACCGTATATAAGT |
| | GCAGTAGTCGCCGTGAACGTTCTTTTTCGCAACGGGTTTGCCGCCAGAACACAGGTAAG |
| | TGCCGTGTGTGGTTCCCGCGGGCCTGGCCTCTTTACGGGTTATGGCCCTTGCGTGCCTT |
| | GAATTACTTCCACCTGGCTGCAGTACGTGATTCTTGATCCCGAGCTTCGGGTTGGAAGT |
| | GGGTGGGAGAGTTCGAGGCCTTGCGCTTAAGGAGCCCCTTCGCCTCGTGCTTGAGTTGA |
| | GGCCTGGCCTGGGCGCTGGGGCCGCCGCGTGCGAATCTGGTGGCACCTTCGCGCCTGTC |
| | TCGCTGCTTTCGATAAGTCTCTAGCCATTTAAAATTTTTGATGACCTGCTGCGACGCTT |
| | TTTTTCTGGCAAGATAGTCTTGTAAATGCGGGCCAAGATCTGCACACTGGTATTTCGGT |
| | TTTTGGGGCCGCGGGCGGCGACGGGGCCCGTGCGTCCCAGCGCACATGTTCGGCGAGGC |
| | GGGGCCTGCGAGCGCGGCCACCGAGAATCGGACGGGGGTAGTCTCAAGCTGGCCGGCCT |
| | GCTCTGGTGCCTGGTCTCGCGCCGCCGTGTATCGCCCCGCCCTGGGCGGCAAGGCTGGC |
| | CCGGTCGGCACCAGTTGCGTGAGCGGAAAGATGGCCGCTTCCCGGCCCTGCTGCAGGGA |
| | GCTCAAAATGGAGGACGCGGCGCTCGGGAGAGCGGGCGGGTGAGTCACCCACACAAAGG |
| | AAAAGGGCCTTTCCGTCCTCAGCCGTCGCTTCATGTGACTCCACGGAGTACCGGGCGCC |
| | GTCCAGGCACCTCGATTAGTTCTCGAGCTTTTGGAGTACGTCGTCTTTAGGTTGGGGGG |
| | AGGGGTTTTATGCGATGGAGTTTCCCCACACTGAGTGGGTGGAGACTGAAGTTAGGCCA |
| | GCTTGGCACTTGATGTAATTCTCCTTGGAATTTGCCCTTTTTGAGTTTGGATCTTGGTT |
| | CATTCTCAAGCCTCAGACAGTGGTTCAAAGTTTTTTTCTTCCATTTCAGGTGTCGTGA |
| | (SEQ ID NO: 20) |
| hCAGG | ACTAGTTATTAATAGTAATCAATTACGGGGTCATTAGTTCATAGCCCATATATGGAGTT |
| | CCGCGTTACATAACTTACGGTAAATGGCCCGCCTGGCTGACCGCCCAACGACCCCCGCC |
| | CATTGACGTCAATAATGACGTATGTTCCCATAGTAACGCCAATAGGGACTTTCCATTGA |
| | CGTCAATGGGTGGAGTATTTACGGTAAACTGCCCACTTGGCAGTACATCAAGTGTATCA |
| | TATGCCAAGTACGCCCCCTATTGACGTCAATGACGGTAAATGGCCCGCCTGGCATTATG |
| | CCCAGTACATGACCTTATGGGACTTTCCTACTTGGCAGTACATCTACGTATTAGTCATC |
| | GCTATTACCATGGTCGAGGTGAGCCCCACGTTCTGCTTCACTCTCCCCATCTCCCCCCC |
| | CTCCCCACCCCCAATTTTGTATTTATTTATTTTTTAATTATTTTGTGCAGCGATGGGGG |
| | GGGGGGGGGGGGGGGGCGCGCGCCAGGCGGGGCGGGGCGGGGCGAGGGGCGGGGCGGGG |
| | CGAGGCGGAGAGGTGCGGCGGCAGCCAATCAGAGCGGCGCGCTCCGAAAGTTTCCTTTT |
| | ATGGCGAGGCGGCGGCGGCGGCGGCCCTATAAAAAGCGAAGCGCGCGGCGGGCGGGGAG |
| | TCGCTGCGACGCTGCCTTCGCCCCGTGCCCCGCTCCGCCGCCGCCTCGCGCCGCCCGCC |
| | CCGGCTCTGACTGACCGCGTTACTCCCACAGGTGAGCGGGCGGGACGGCCCTTCTCCTC |
| | CGGGCTGTAATTAGCGCTTGGTTTAATGACGGCTTGTTTCTTTTCTGTGGCTGCGTGAA |
| | AGCCTTGAGGGGCTCCGGGAGGGCCCTTTGTGCGGGGGGAGCGGCTCGGGGGGTGCGTG |
| | CGTGTGTGTGTGCGTGGGGAGCGCCGCGTGCGGCTCCGCGCTGCCCGGCGGCTGTGAGC |
| | GCTGCGGGCGCGGCGCGGGGCTTTGTGCGCTCCGCAGTGTGCGCGAGGGGAGCGCGGCC |
| | GGGGGCGGTGCCCCGCGGTGCGGGGGGGGCTGCGAGGGGAACAAAGGCTGCGTGCGGGG |
| | TGTGTGCGTGGGGGGGTGAGCAGGGGGTGTGGGCGCGTCGGTCGGGCTGCAACCCCCCC |
| | TGCACCCCCCTCCCCGAGTTGCTGAGCACGGCCCGGCTTCGGGTGCGGGGCTCCGTACG |
| | GGGCGTGGCGCGGGGCTCGCCGTGCCGGGCGGGGGGTGGCGGCAGGTGGGGGTGCCGGG |
| | CGGGGCGGGGCCGCCTCGGGCCGGGGAGGGCTCGGGGGAGGGGCGCGGCGGCCCCCGGA |
| | GCGCCGGCGGCTGTCGAGGCGCGGCGAGCCGCAGCCATTGCCTTTTATGGTAATCGTGC |
| | GAGAGGGCGCAGGGACTTCCTTTGTCCCAAATCTGTGCGGAGCCGAAATCTGGGAGGCG |
| | CCGCCGCACCCCCTCTAGCGGGCGCGGGGCGAAGCGGTGCGGCGCCGGCAGGAAGGAAA |
| | TGGGCGGGGAGGGCCTTCGTGCGTCGCCGCGCCGCCGTCCCCTTCTCCCTCTCCAGCCT |
| | CGGGGCTGTCCGCGGGGGGACGGCTGCCTTCGGGGGGGACGGGGCAGGGCGGGGTTCGG |
| | CTTCTGGCGTGTGACCGGCGGCTCTAGAGCCTCTGCTAACCATGTTCATGCCTTCTTCT |
| | TTTTCCTACAGCTCCTGGGCAACGTGCTGGTTATTGTGCTGTCTCATCATTTTGGCAAA |
| | GAATTC (SEQ ID NO: 21) |
| hEF1aV2 | Gggcagagcgcacatcgcccacagtccccgagaagttggggggaggggtcggcaattga |
| | accggtgcctagagaaggtggcgcggggtaaactgggaaagtgatgtcgtgtactggct |
| | ccgcctttttcccgaggggggggagaaccgtatataagtgcagtagtcgccgtgaacgt |
| | tctttttcgcaacgggtttgccgccagaacacag (SEQ ID NO: 22) |
| hACTb | CCACTAGTTCCATGTCCTTATATGGACTCATCTTTGCCTATTGCGACACACACTCAATG |
| | AACACCTACTACGCGCTGCAAAGAGCCCCGCAGGCCTGAGGTGCCCCCACCTCACCACT |
| | CTTCCTATTTTTGTGTAAAAATCCAGCTTCTTGTCACCACCTCCAAGGAGGGGGAGGAG |
| | GAGGAAGGCAGGTTCCTCTAGGCTGAGCCGAATGCCCCTCTGTGGTCCCACGCCACTGA |
| | TCGCTGCATGCCCACCACCTGGGTACACACAGTCTGTGATTCCCGGAGCAGAACGGACC |
| | CTGCCCACCCGGTCTTGTGTGCTACTCAGTGGACAGACCCAAGGCAAGAAAGGGTGACA |
| | AGGACAGGGTCTTCCCAGGCTGGCTTTGAGTTCCTAGCACCGCCCCGCCCCCAATCCTC |
| | TGTGGCACATGGAGTCTTGGTCCCCAGAGTCCCCCAGCGGCCTCCAGATGGTCTGGGAG |
| | GGCAGTTCAGCTGTGGCTGCGCATAGCAGACATACAACGGACGGTGGGCCCAGACCCAG |
| | GCTGTGTAGACCCAGCCCCCCCGCCCCGCAGTGCCTAGGTCACCCACTAACGCCCCAGG |
| | CCTGGTCTTGGCTGGGCGTGACTGTTACCCTCAAAAGCAGGCAGCTCCAGGGTAAAAGG |
| | TGCCCTGCCCTGTAGAGCCCACCTTCCTTCCCAGGGCTGCGGCTGGGTAGGTTTGTAGC |
| | CTTCATCACGGGCCACCTCCAGCCACTGGACCGCTGGCCCCTGCCCTGTCCTGGGGAGT |
| | GTGGTCCTGCGACTTCTAAGTGGCCGCAAGCCACCTGACTCCCCCAACACCACACTCTA |
| | CCTCTCAAGCCCAGGTCTCTCCCTAGTGACCCACCCAGCACATTTAGCTAGCTGAGCCC |
| | CACAGCCAGAGGTCCTCAGGCCCTGCTTTCAGGGCAGTTGCTCTGAAGTCGGCAAGGGG |
| | GAGTGACTGCCTGGCCACTCCATGCCCTCCAAGAGCTCCTTCTGCAGGAGCGTACAGAA |
| | CCCAGGGCCCTGGCACCCGTGCAGACCCTGGCCCACCCCACCTGGGCGCTCAGTGCCCA |
| | AGAGATGTCCACACCTAGGATGTCCCGCGGTGGGTGGGGGGCCCGAGAGACGGGCAGGC |
| | CGGGGGCAGGCCTGGCCATGCGGGGCCGAACCGGGCACTGCCCAGCGTGGGGCGCGGGG |
| | GCCACGGCGCGCGCCCCCAGCCCCCGGGCCCAGCACCCCAAGGCGGCCAACGCCAAAAC |
| | TCTCCCTCCTCCTCTTCCTCAATCTCGCTCTCGCTCTTTTTTTTTTTCGCAAAAGGAGG |
| | GGAGAGGGGGTAAAAAAATGCTGCACTGTGCGGCGAAGCCGGTGAGTGAGCGGCGCGGG |
| | GCCAATCAGCGTGCGCCGTTCCGAAAGTTGCCTTTTATGGCTCGAGCGGCCGCGGCGGC |
| | GCCCTATAAAACCCAGCGGCGCGACGCGCCACCACCGCCGAGACCGCGTCCGCCCCGCG |
| | AGCACAGAGCCTCGCCTTTGCCGATCCGCCGCCCGTCCACACCCGCCGCCAGgtaagcc |
| | cggccagccgaccggggcaggcggctcacggcccggccgcaggcggccgcggccccttc |
| | gcccgtgcagagccgccgtctgggccgcagcggggggcgcatggggggggaaccggacc |
| | gccgtggggggcgcgggagaagcccctgggcctccggagatgggggacaccccacgcca |
| | gttcggaggcgcgaggccgcgctcgggaggcgcgctccgggggtgccgctctcggggcg |
| | ggggcaaccggcggggtctttgtctgagccgggctcttgccaatggggatcgcaggggg |
| | gcgcggcggagcccccgccaggcccggtgggggctggggcgccattgcgcgtgcgcgct |
| | ggtcctttgggcgctaactgcgtgcgcgctgggaattggcgctaattgcgcgtgcgcgc |
| | tgggactcaaggcgctaactgcgcgtgcgttctggggcccggggtgccgcggcctgggc |
| | tggggcgaaggcgggctcggccggaaggggggggtcgccgcggctcccgggcgcttgcg |
| | cgcacttcctgcccgagccgctggccgcccgagggtgtggccgctgcgtgcgcgcgcgc |
| | cgacccggcgctgtttgaaccgggcggaggcggggctggcgcccggttgggagggggtt |
| | ggggcctggcttcctgccgcgcgccgcggggacgcctccgaccagtgtttgccttttat |
| | ggtaataacgcggccggcccggcttcctttgtccccaatctgggcgcgcgccggcgccc |
| | cctggcggcctaaggactcggcgcgccggaagtggccagggcgggggcgacctcggctc |
| | acagcgcgcccggctat (SEQ ID NO: 23) |
| heIF4A1 | GTTGATTTCCTTCATCCCTGGCACACGTCCAGGCAGTGTCGAATCCATCTCTGCTACAG |
| | GGGAAAACAAATAACATTTGAGTCCAGTGGAGACCGGGAGCAGAAGTAAAGGGAAGTGA |
| | TAACCCCCAGAGCCCGGAAGCCTCTGGAGGCTGAGACCTCGCCCCCCTTGCGTGATAGG |
| | GCCTACGGAGCCACATGACCAAGGCACTGTCGCCTCCGCACGTGTGAGAGTGCAGGGCC |
| | CCAAGATGGCTGCCAGGCCTCGAGGCCTGACTCTTCTATGTCACTTCCGTACCGGCGAG |
| | AAAGGCGGGCCCTCCAGCCAATGAGGCTGCGGGGGGGGCCTTCACCTTGATAGGCACTC |
| | GAGTTATCCAATGGTGCCTGCGGGCCGGAGCGACTAGGAACTAACGTCATGCCGAGTTG |
| | CTGAGCGCCGGCAGGCGGGGCCGGGGCGGCCAAACCAATGCGATGGCCGGGGCGGAGTC |
| | GGGCGCTCTATAAGTTGTCGATAGGCGGGCACTCCGCCCTAGTTTCTAAGGACCATG |
| | (SEQ ID NO: 24) |
| hGAPDH | AGTTCCCCAACTTTCCCGCCTCTCAGCCTTTGAAAGAAAGAAAGGGGAGGGGGCAGGCC |
| | GCGTGCAGTCGCGAGCGGTGCTGGGCTCCGGCTCCAATTCCCCATCTCAGTCGCTCCCA |
| | AAGTCCTTCTGTTTCATCCAAGCGTGTAAGGGTCCCCGTCCTTGACTCCCTAGTGTCCT |
| | GCTGCCCACAGTCCAGTCCTGGGAACCAGCACCGATCACCTCCCATCGGGCCAATCTCA |
| | GTCCCTTCCCCCCTACGTCGGGGCCCACACGCTCGGTGCGTGCCCAGTTGAACCAGGCG |
| | GCTGCGGAAAAAAAAAAGCGGGGAGAAAGTAGGGCCCGGCTACTAGCGGTTTTACGGGC |
| | GCACGTAGCTCAGGCCTCAAGACCTTGGGCTGGGACTGGCTGAGCCTGGCGGGAGGCGG |
| | GGTCCGAGTCACCGCCTGCCGCCGCGCCCCCGGTTTCTATAAATTGAGCCCGCAGCCTC |
| | CCGCTTCGCTCTCTGCTCCTCCTGTTCGACAGTCAGCCGCATCTTCTTTTGCGTCGCCA |
| | Ggtgaagacgggcggagagaaacccgggaggctagggacggcctgaaggcggcaggggg |
| | gggcaggccggatgtgttcgcgccgctgcggggtgggcccgggcggcctccgcattgca |
| | ggggcgggcggaggacgtgatgcggcgcgggctgggcatggaggcctggtgggggaggg |
| | gaggggaggcgtgggtgtcggccggggccactaggcgctcactgttctctccctccgcg |
| | cagCCGAGCCACATCGCTGAGACAC (SEQ ID NO: 25) |
| hGRP78 | AGTGCGGTTACCAGCGGAAATGCCTCGGGGTCAGAAGTCGCAGGAGAGATAGACAGCTG |
| | CTGAACCAATGGGACCAGCGGATGGGGCGGATGTTATCTACCATTGGTGAACGTTAGAA |
| | ACGAATAGCAGCCAATGAATCAGCTGGGGGGGCGGAGCAGTGACGTTTATTGCGGAGGG |
| | GGCCGCTTCGAATCGGCGGCGGCCAGCTTGGTGGCCTGGGCCAATGAACGGCCTCCAAC |
| | GAGCAGGGCCTTCACCAATCGGCGGCCTCCACGACGGGGCTGGGGGAGGGTATATAAGC |
| | CGAGTAGGCGACGGTGAGGTCGACGCCGGCCAAGACAGCACAGACAGATTGACCTATTG |
| | GGGTGTTTCGCGAGTGTGAGAGGGAAGCGCCGCGGCCTGTATTTCTAGACCTGCCCTTC |
| | GCCTGGTTCGTGGCGCCTTGTGACCCCGGGCCCCTGCCGCCTGCAAGTCGGAAATTGCG |
| | CTGTGCTCCTGTGCTACGGCCTGTGGCTGGACTGCCTGCTGCTGCCCAACTGGCTGGCA |
| | C (SEQ ID NO: 26) |
| hGRP94 | TAGTTTCATCACCACCGCCACCCCCCCGCCCCCCCGCCATCTGAAAGGGTTCTAGGGGA |
| | TTTGCAACCTCTCTCGTGTGTTTCTTCTTTCCGAGAAGCGCCGCCACACGAGAAAGCTG |
| | GCCGCGAAAGTCGTGCTGGAATCACTTCCAACGAAACCCCAGGCATAGATGGGAAAGGG |
| | TGAAGAACACGTTGCCATGGCTACCGTTTCCCCGGTCACGGAATAAACGCTCTCTAGGA |
| | TCCGGAAGTAGTTCCGCCGCGACCTCTCTAAAAGGATGGATGTGTTCTCTGCTTACATT |
| | CATTGGACGTTTTCCCTTAGAGGCCAAGGCCGCCCAGGCAAAGGGGCGGTCCCACGCGT |
| | GAGGGGCCCGCGGAGCCATTTGATTGGAGAAAAGCTGCAAACCCTGACCAATCGGAAGG |
| | AGCCACGCTTCGGGCATCGGTCACCGCACCTGGACAGCTCCGATTGGTGGACTTCCGCC |
| | CCCCCTCACGAATCCTCATTGGGTGCCGTGGGTGCGTGGTGCGGCGCGATTGGTGGGTT |
| | CATGTTTCCCGTCCCCCGCCCGCGAGAAGTGGGGGTGAAAAGCGGCCCGACCTGCTTGG |
| | GGTGTAGTGGGCGGACCGCGCGGCTGGAGGTGTGAGGATCCGAACCCAGGGGTGGGGGG |
| | TGGAGGCGGCTCCTGCGATCGAAGGGGACTTGAGACTCACCGGCCGCACGTC (SEQ |
| | ID NO: 27) |
| hHSP70 | GGGCCGCCCACTCCCCCTTCCTCTCAGGGTCCCTGTCCCCTCCAGTGAATCCCAGAAGA |
| | CTCTGGAGAGTTCTGAGCAGGGGGCGGCACTCTGGCCTCTGATTGGTCCAAGGAAGGCT |
| | GGGGGGCAGGACGGGAGGCGAAAACCCTGGAATATTCCCGACCTGGCAGCCTCATCGAG |
| | CTCGGTGATTGGCTCAGAAGGGAAAAGGCGGGTCTCCGTGACGACTTATAAAAGCCCAG |
| | GGGCAAGCGGTCCGGATAACGGCTAGCCTGAGGAGCTGCTGCGACAGTCCACTACCTTT |
| | TTCGAGAGTGACTCCCGTTGTCCCAAGGCTTCCCAGAGCGAACCTGTGCGGCTGCAGGC |
| | ACCGGCGCGTCGAGTTTCCGGCGTCCGGAAGGACCGAGCTCTTCTCGCGGATCCAGTGT |
| | TCCGTTTCCAGCCCCCAATCTCAGAGCGGAGCCGACAGAGAGCAGGGAACCC (SEQ |
| | ID NO: 28) |
| hKINb | GCCCCACCCCCGTCCGCGTTACAACCGGGAGGCCCGCTGGGTCCTGCACCGTCACCCTC |
| | CTCCCTGTGACCGCCCACCTGATACCCAAACAACTTTCTCGCCCCTCCAGTCCCCAGCT |
| | CGCCGAGCGCTTGCGGGGAGCCACCCAGCCTCAGTTTCCCCAGCCCCGGGCGGGGCGAG |
| | GGGCGATGACGTCATGCCGGCGCGCGGCATTGTGGGGCGGGGCGAGGCGGGGCGCCGGG |
| | GGGAGCAACACTGAGACGCCATTTTCGGCGGCGGGAGCGGCGCAGGCGGCCGAGCGGGA |
| | CTGGCTGGGTCGGCTGGGCTGCTGGTGCGAGGAGCCGCGGGGCTGTGCTCGGCGGCCAA |
| | GGGGACAGCGCGTGGGTGGCCGAGGATGCTGCGGGGCGGTAGCTCCGGCGCCCCTCGCT |
| | GGTGACTGCTGCGCCGTGCCTCACACAGCCGAGGCGGGCTCGGCGCACAGTCGCTGCTC |
| | CGCGCTCGCGCCCGGCGGCGCTCCAGGTGCTGACAGCGCGAGAGAGCGCGGCCTCAGGA |
| | GCAACAC (SEQ ID NO: 29) |
| hUBIb | TTCCAGAGCTTTCGAGGAAGGTTTCTTCAACTCAAATTCATCCGCCTGATAATTTTCTT |
| | ATATTTTCCTAAAGAAGGAAGAGAAGCGCATAGAGGAGAAGGGAAATAATTTTTTAGGA |
| | GCCTTTCTTACGGCTATGAGGAATTTGGGGCTCAGTTGAAAAGCCTAAACTGCCTCTCG |
| | GGAGGTTGGGCGCGGCGAACTACTTTCAGCGGCGCACGGAGACGGCGTCTACGTGAGGG |
| | GTGATAAGTGACGCAACACTCGTTGCATAAATTTGCGCTCCGCCAGCCCGGAGCATTTA |
| | GGGGCGGTTGGCTTTGTTGGGTGAGCTTGTTTGTGTCCCTGTGGGTGGACGTGGTTGGT |
| | GATTGGCAGGATCCTGGTATCCGCTAACAGgtactggcccacagccgtaaagacctgcg |
| | ggggcgtgagaggggggaatgggtgaggtcaagctggaggcttcttggggttgggtggg |
| | ccgctgaggggaggggagggcgaggtgacgcgacacccggcctttctgggagagtgggc |
| | cttgttgacctaaggggggcgagggcagttggcacgcgcacgcgccgacagaaactaac |
| | agacattaaccaacagegattccgtcgcgtttacttgggaggaaggcggaaaagaggta |
| | gtttgtgtggcttctggaaaccctaaatttggaatcccagtatgagaatggtgtccctt |
| | cttgtgtttcaatgggatttttacttcgcgagtcttgtgggtttggttttgttttcagt |
| | ttgcctaacaccgtgcttaggtttgaggcagattggagttcggtcgggggagtttgaat |
| | atccggaacagttagtggggaaagctgtggacgcttggtaagagagcgctctggatttt |
| | ccgctgttgacgttgaaaccttgaatgacgaatttcgtattaagtgacttagccttgta |
| | aaattgaggggaggcttgcggaatattaacgtatttaaggcattttgaaggaatagttg |
| | ctaattttgaagaatattaggtgtaaaagcaagaaatacaatgatcctgaggtgacacg |
| | cttatgttttacttttaaactagGTCACC (SEQ ID NO: 30) |
| CAG | gacattgattattgactagttattaatagtaatcaattacggggtcattagttcatagc |
| | ccatatatggagttccgcgttacataacttacggtaaatggcccgcctggctgaccgcc |
| | caacgacccccgcccattgacgtcaataatgacgtatgttcccatagtaacgccaatag |
| | ggactttccattgacgtcaatgggtggagtatttacggtaaactgcccacttggcagta |
| | catcaagtgtatcatatgccaagtacgccccctattgacgtcaatgacggtaaatggcc |
| | cgcctggcattatgcccagtacatgaccttatgggactttcctacttggcagtacatct |
| | acgtattagtcatcgctattaccatggtcgaggtgagccccacgttctgcttcactctc |
| | cccatctcccccccctccccacccccaattttgtatttatttattttttaattattttg |
| | tgcagcgatgggggggggggggggggggggcgcgcgccaggcggggcggggcggggcga |
| | ggggcggggcggggcgaggcggagaggtgcggcggcagccaatcagagcggcgcgctcc |
| | gaaagtttccttttatggcgaggcggcggcggcggcggccctataaaaagcgaagcgcg |
| | cggcgggcg (SEQ ID NO: 173) |
| HLP | Tgtttgctgcttgcaatgtttgcccattttagggtggacacaggacgctgtggtttctg |
| | agccagggggcgactcagatcccagccagtggacttagcccctgtttgctcctccgata |
| | actggggtgaccttggttaatattcaccagcagcctcccccgttgcccctctggatcca |
| | ctgcttaaatacggacgaggacagggccctgtctcctcagcttcaggcaccaccactga |
| | cctgggacagtgaat (SEQ ID NO: 174) |

The promoter can be a tissue-specific promoter. In general, a tissue-specific promoter directs transcription of a nucleic acid, (e.g., the engineered nucleic acids encoding the chimeric proteins, such as membrane-cleavable chimeric proteins having the formula S—C-MT or MT-C—S) such that expression is limited to a specific cell type, organelle, or tissue. Tissue-specific promoters include, but are not limited to, albumin (liver specific, Pinkert et al., (1987)), lymphoid specific promoters (Calame and Eaton, 1988), particular promoters of T-cell receptors (Winoto and Baltimore, (1989)) and immunoglobulins; Banerji et al., (1983); Queen and Baltimore, 1983), neuron specific promoters (e.g. the neurofilament promoter; Byrne and Ruddle, 1989), pancreas specific promoters (Edlund et al., (1985)) or mammary gland specific promoters (milk whey promoter, U.S. Pat. No. 4,873,316 and European Application Publication No. 264,166) as well as developmentally regulated promoters such as the murine hox promoters (Kessel and Gruss, Science 249:374-379 (1990)) or the α-fetoprotein promoter (Campes and Tilghman, Genes Dev. 3:537-546 (1989)), the contents of each of which are fully incorporated by reference herein. The promoter can be constitutive in the respective specific cell type, organelle, or tissue. Tissue-specific promoters and/or regulatory elements can also include promoters from the liver fatty acid binding (FAB) protein gene, specific for colon epithelial cells; the insulin gene, specific for pancreatic cells; the transphyretin, .alpha.1-antitrypsin, plasminogen activator inhibitor type 1 (PAI-I), apolipoprotein AI and LDL receptor genes, specific for liver cells; the myelin basic protein (MBP) gene, specific for oligodendrocytes; the glial fibrillary acidic protein (GFAP) gene, specific for glial cells; OPSIN, specific for targeting to the eye; and the neural-specific enolase (NSE) promoter that is specific for nerve cells. Examples of tissue-specific promoters include, but are not limited to, the promoter for creatine kinase, which has been used to direct expression in muscle and cardiac tissue and immunoglobulin heavy or light chain promoters for expression in B cells. Other tissue specific promoters include the human smooth muscle alpha-actin promoter. Exemplary tissue-specific expression elements for the liver include but are not limited to HMG-COA reductase promoter, sterol regulatory element 1, phosphoenol pyruvate carboxy kinase (PEPCK) promoter, human C-reactive protein (CRP) promoter, human glucokinase promoter, cholesterol L 7-alpha hydroylase (CYP-7) promoter, beta-galactosidase alpha-2,6 sialylkansferase promoter, insulin-like growth factor binding protein (IGFBP-I) promoter, aldolase B promoter, human transferrin promoter, and collagen type I promoter. Exemplary tissue-specific expression elements for the prostate include but are not limited to the prostatic acid phosphatase (PAP) promoter, prostatic secretory protein of 94 (PSP 94) promoter, prostate specific antigen complex promoter, and human glandular kallikrein gene promoter (hgt-1). Exemplary tissue-specific expression elements for gastric tissue include but are not limited to the human H+/K+-ATPase alpha subunit promoter. Exemplary tissue-specific expression elements for the pancreas include but are not limited to pancreatitis associated protein promoter (PAP), elastase 1 transcriptional enhancer, pancreas specific amylase and elastase enhancer promoter, and pancreatic cholesterol esterase gene promoter. Exemplary tissue-specific expression elements for the endometrium include, but are not limited to, the uteroglobin promoter. Exemplary tissue-specific expression elements for adrenal cells include, but are not limited to, cholesterol side-chain cleavage (SCC) promoter. Exemplary tissue-specific expression elements for the general nervous system include, but are not limited to, gamma-gamman enolase (neuron-specific enolase, NSE) promoter. Exemplary tissue-specific expression elements for the brain include, but are not limited to, the neurofilament heavy chain (NF-H) promoter. Exemplary tissue-specific expression elements for lymphocytes include, but are not limited to, the human CGL-1/granzyme B promoter, the terminal deoxy transferase (TdT), lambda 5, VpreB, and lck (lymphocyte specific tyrosine protein kinase p561ck) promoter, the humans CD2 promoter and its 3′transcriptional enhancer, and the human NK and T cell specific activation (NKG5) promoter. Exemplary tissue-specific expression elements for the colon include, but are not limited to, pp60c-src tyrosine kinase promoter, organ-specific neoantigens (OSNs) promoter, and colon specific antigen-P promoter. Tissue-specific expression elements for breast cells are for example, but are not limited to, the human alpha-lactalbumin promoter. Exemplary tissue-specific expression elements for the lung include, but are not limited to, the cystic fibrosis transmembrane conductance regulator (CFTR) gene promoter.

In some embodiments, a promoter of the present disclosure is modulated by signals within a tumor microenvironment. A tumor microenvironment is considered to modulate a promoter if, in the presence of the tumor microenvironment, the activity of the promoter is increased or decreased by at least 10%, relative to activity of the promoter in the absence of the tumor microenvironment. In some embodiments, the activity of the promoter is increased or decreased by at least 20%, at least 30%, at least 40%, at least 50%, at least 60%, at least 70%, at least 80%, at least 90%, at least 100%, relative to activity of the promoter in the absence of the tumor microenvironment. For example, the activity of the promoter is increased or decreased by 10-20%, 10-30%, 10-40%, 10-50%, 10-60%, 10-70%, 10-80%, 10-90%, 10-100%, 10-200%, 20-30%, 20-40%, 20-50%, 20-60%, 20-70%, 20-80%, 20-90%, 20-100%, 20-200%, 50-60%, 50-70%, 50-80%, 50-90%, 50-100%, or 50-200%, relative to activity of the promoter in the absence of the tumor microenvironment.

In some embodiments, the activity of the promoter is increased or decreased by at least 2 fold (e.g., 2, 3, 4, 5, 10, 25, 20, 25, 50, or 100 fold), relative to activity of the promoter in the absence of the tumor microenvironment. For example, the activity of the promoter is increased or decreased by at least 3 fold, at least 5 fold, at least 10 fold, at least 20 fold, at least 50 fold, or at least 100 fold, relative to activity of the promoter in the absence of the tumor microenvironment. In some embodiments, the activity of the promoter is increased or decreased by 2-10, 2-20, 2-30, 2-40, 2-50, 2-60, 2-70, 2-80, 2-90, or 2-100 fold, relative to activity of the promoter in the absence of the tumor microenvironment.

In some embodiments, a promoter of the present disclosure is activated under a hypoxic condition. A “hypoxic condition” is a condition where the body or a region of the body is deprived of adequate oxygen supply at the tissue level. Hypoxic conditions can cause inflammation (e.g., the level of inflammatory cytokines increase under hypoxic conditions). In some embodiments, the promoter that is activated under hypoxic condition is operably linked to a nucleotide encoding a chimeric proteins that decreases the expression of activity of inflammatory cytokines, thus reducing the inflammation caused by the hypoxic condition. In some embodiments, the promoter that is activated under hypoxic conditions comprises a hypoxia responsive element (HRE). A “hypoxia responsive element (HRE)” is a response element that responds to hypoxia-inducible factor (HIF). The HRE, in some embodiments, comprises a consensus motif NCGTG (where N is either A or G).

### Activation-Conditional Control Polypeptide (ACP) Promoter Systems

In some embodiments, a synthetic promoter is a promoter system including an activation-conditional control polypeptide-(ACP-)binding domain sequence and a promoter sequence. Such a system is also referred to herein as an “ACP-responsive promoter.” In general, an ACP promoter system includes a first expression cassette encoding an activation-conditional control polypeptide (ACP) and a second expression cassette encoding an ACP-responsive promoter operably linked to an exogenous polynucleotide sequence, such as the exogenous polynucleotide sequence encoding the membrane-cleavable chimeric proteins described herein or any other protein of interest (e.g., a protease). In some embodiments, the first expression cassette and second expression cassette are each encoded by a separate engineered nucleic acid. In other embodiments, the first expression cassette and the second expression cassette are encoded by the same engineered nucleic acid. The ACP-responsive promoter can be operably linked to a nucleotide sequence encoding a single protein of interest or multiple proteins of interest.

The promoters of the ACP promoter system, e.g., either a promoter driving expression of the ACP or the promoter sequence of the ACP-responsive promoter, can include any of the promoter sequences described herein (see “Promoters” above). The ACP-responsive promoter can be derived from minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTK, inducer molecule responsive promoters, and tandem repeats thereof. In some embodiments, the ACP-responsive promoter includes a minimal promoter.

In some embodiments, the ACP-binding domain includes one or more zinc finger binding sites. In some embodiments, the ACP-responsive promoter includes a minimal promoter and the ACP-binding domain includes one or more zinc finger binding sites. The ACP-binding domain can include 1, 2, 3, 4, 5, 6, 7, 8, 9, 10 or more zinc finger binding sites. In some embodiments, the transcription factor is a zinc-finger-containing transcription factor. In some embodiments, the zinc-finger-containing transcription factor is a synthetic transcription factor. In some embodiments, the ACP-binding domain includes one or more zinc finger binding sites and the ACP has a DNA-binding zinc finger protein domain (ZF protein domain). In some embodiments, the ACP has a DNA-binding zinc finger protein domain (ZF protein domain) and an effector domain. In some embodiments, the ACP-binding domain includes one or more zinc finger binding sites and the ACP has a DNA-binding zinc finger protein domain (ZF protein domain) and an effector domain. In some embodiments, the ZF protein domain is modular in design and is composed of zinc finger arrays (ZFA). A zinc finger array comprises multiple zinc finger protein motifs that are linked together. Each zinc finger motif binds to a different nucleic acid motif. This results in a ZFA with specificity to any desired nucleic acid sequence, e.g., a ZFA with desired specificity to an ACP-binding domain having a specific zinc finger binding site composition and/or configuration. The ZF motifs can be directly adjacent to each other, or separated by a flexible linker sequence. In some embodiments, a ZFA is an array, string, or chain of ZF motifs arranged in tandem. A ZFA can have 1, 2, 3, 4, 5, 6, 7, 8, 9, 10, 11, 12, 13, 14, or 15 zinc finger motifs. The ZFA can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, or 5-15 zinc finger motifs. The ZF protein domain can have 1, 2, 3, 4, 5, 6, 7, 8, 9, 10, 11, 12, 13, 14, 15, or more ZFAs. The ZF domain can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, or 5-15 ZFAs. In some embodiments, the ZF protein domain comprises one to ten ZFA(s). In some embodiments, the ZF protein domain comprises at least one ZFA. In some embodiments, the ZF protein domain comprises at least two ZFAs. In some embodiments, the ZF protein domain comprises at least three ZFAs. In some embodiments, the ZF protein domain comprises at least four ZFAs. In some embodiments, the ZF protein domain comprises at least five ZFAs. In some embodiments, the ZF protein domain comprises at least ten ZFAs.

In some embodiments, the ACP is a transcriptional modulator. In some embodiments, the ACP is a transcriptional repressor. In some embodiments, the ACP is a transcriptional activator. In some embodiments, the ACP is a transcription factor. In some embodiments, the ACP comprises a DNA-binding domain and a transcriptional effector domain. In some embodiments, the DNA-binding domain comprises a tetracycline (or derivative thereof) repressor (TetR) domain. In some embodiments, the ACP is an antigen recognizing receptor of the present disclosure.

The ACP can also further include an effector domain, such as a transcriptional effector domain. For instance, a transcriptional effector domain can be the effector or activator domain of a transcription factor. Transcription factor activation domains are also known as transactivation domains, and act as scaffold domains for proteins such as transcription coregulators that act to activate or repress transcription of genes. Any suitable transcriptional effector domains can be used in the ACP including, but not limited to, a Herpes Simplex Virus Protein 16 (VP16) activation domain; an activation domain consisting of four tandem copies of VP16, a VP64 activation domain; a p65 activation domain of NFκB; an Epstein-Barr virus R transactivator (Rta) activation domain; a tripartite activator comprising the VP64, the p65, and the Rta activation domains, the tripartite activator is known as a VPR activation domain; a histone acetyltransferase (HAT) core domain of the human E1A-associated protein p300, known as a p300 HAT core activation domain; a Krüppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain, or any combination thereof.

In some embodiments, the effector domain is s transcription effector domain selected from: a Herpes Simplex Virus Protein 16 (VP16) activation domain; an activation domain consisting of four tandem copies of VP16, a VP64 activation domain; a p65 activation domain of NFκB; an Epstein-Barr virus R transactivator (Rta) activation domain; a tripartite activator comprising the VP64, the p65, and the Rta activation domains, the tripartite activator is known as a VPR activation domain; a histone acetyltransferase (HAT) core domain of the human E1A-associated protein p300, known as a p300 HAT core activation domain; a Krüppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

In some embodiments, the ACP is a small molecule (e.g., drug) inducible polypeptide. For example, in some embodiments, the ACP may be induced by tetracycline (or derivative thereof), and comprises a TetR domain and a VP16 effector domain. In some embodiments, the ACP includes an estrogen receptor variant, such as ERT2, and may be regulated by tamoxifen, or a metabolite thereof (such as 4-hydroxy-tamoxifen [4-OHT], N-desmethyltamoxifen, tamoxifen-N-oxide, or endoxifen), through tamoxifen-controlled nuclear localization.

In some embodiments, the ACP is a small molecule (e.g., drug) inducible polypeptide that includes a repressible protease and one or more cognate cleavage sites of the repressible protease. In some embodiments, a repressible protease is active (cleaves a cognate cleavage site) in the absence of the specific agent and is inactive (does not cleave a cognate cleavage site) in the presence of the specific agent. In some embodiments, the specific agent is a protease inhibitor. In some embodiments, the protease inhibitor specifically inhibits a given repressible protease of the present disclosure. The repressible protease can be any of the proteases described herein that is capable of inactivation by the presence or absence of a specific agent (see “Protease Cleavage Site” above for exemplary repressible proteases, cognate cleavage sites, and protease inhibitors).

In some embodiments, the ACP has a degron domain (see “Degron Systems and Domains” above for exemplary degron sequences). The degron domain can be in any order or position relative to the individual domains of the ACP. For example, the degron domain can be N-terminal of the repressible protease, C-terminal of the repressible protease, N-terminal of the ZF protein domain, C-terminal of the ZF protein domain, N-terminal of the effector domain, or C-terminal of the effector domain.

### Multicistronic and Multiple Promoter Systems

In some embodiments, engineered nucleic acids (e.g., an engineered nucleic acid comprising an expression cassette) are configured to produce multiple chimeric proteins. For example, nucleic acids may be configured to produce 2-20 different chimeric proteins. In some embodiments, nucleic acids are configured to produce 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 chimeric proteins. In some embodiments, nucleic acids are configured to produce 1, 2, 3, 4, 5, 6, 7, 8, 9, 10, 11, 12, 13, 14, 15, 16, 17, 18, 19, or 20 chimeric proteins.

In general, the engineered nucleic acids described herein are multicistronic, i.e., as described above.

Engineered nucleic acids can also use multiple promoters to express genes from multiple ORFs, i.e., more than one separate mRNA transcripts can be produced from a single engineered nucleic acid. For example, a first promoter can be operably linked to a polynucleotide sequence encoding a first chimeric protein, and a second promoter can be operably linked to a polynucleotide sequence encoding a second chimeric protein. In general, any number of promoters can be used to express any number of chimeric proteins. In some embodiments, at least one of the ORFs expressed from the multiple promoters can be multicistronic.

“Linkers,” as used herein can refer to polypeptides that link a first polypeptide sequence and a second polypeptide sequence, the multicistronic linkers described above, or the additional promoters that are operably linked to additional ORFs described above.

### Engineered Cells

Provided herein are engineered cells, and methods of producing the engineered cells, that produce each of (i) a membrane-cleavable chimeric protein; (ii) a bivalent aCAR; and (iii) an iCAR. In general, engineered cells of the present disclosure may be engineered to express the chimeric proteins provided for herein, such as each of the membrane-cleavable chimeric proteins, the bivalent aCARs, and the iCARs described herein. These cells are referred to herein as “engineered cells.” These cells, which typically contain engineered nucleic acid, do not occur in nature. In some embodiments, the cells are engineered to include a nucleic acid comprising a promoter operably linked to a nucleotide sequence encoding a chimeric protein, for example, a membrane-cleavable chimeric protein. An engineered cell can comprise an engineered nucleic acid integrated into the cell's genome. An engineered cell can comprise an engineered nucleic acid capable of expression without integrating into the cell's genome, for example, engineered with a transient expression system such as a plasmid or mRNA.

The present disclosure also encompasses additivity and synergy between a chimeric protein(s) and the engineered cell from which they are produced. In some embodiments, cells are engineered to produce at least two (e.g., 2, 3, 4, 5, 6, 7, 8, 9, 10 or more) chimeric proteins, for example ate least two membrane-cleavable chimeric proteins. In other embodiments, cells are engineered to produce at least one chimeric proteins having an effector molecule that is not natively produced by the cells. Such an effector molecule may, for example, complement the function of effector molecules natively produced by the cells.

In some embodiments, cells are engineered to express membrane-tethered anti-CD3 and/or anti-CD28 agonist extracellular domains.

In some embodiments, a cell (e.g., an immune cell or a stem cell) is engineered to produce multiple chimeric proteins. For example, cells may be engineered to produce 2-20 different chimeric proteins, such as 2-20 different membrane-cleavable chimeric proteins. In some embodiments, cells engineered to produce 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 chimeric proteins. In some embodiments, cells are engineered to produce 1, 2, 3, 4, 5, 6, 7, 8, 9, 10, 11, 12, 13, 14, 15, 16, 17, 18, 19, or 20 chimeric proteins.

In some embodiments, engineered cells comprise one or more engineered nucleic acids encoding a promoter operably linked to a nucleotide sequence encoding a chimeric protein. In some embodiments, cells are engineered to include a plurality of engineered nucleic acids, e.g., at least two engineered nucleic acids, each encoding a promoter operably linked to a nucleotide sequence encoding at least one (e.g., 1, 2 or 3) chimeric protein. For example, cells may be engineered to comprise at least 2, at least 3, at least 4, at least 5, at least 6, at least 7, at least 8, at least 8, at least 9, or at least 10, engineered nucleic acids, each encoding a promoter operably linked to a nucleotide sequence encoding at least one (e.g., 1, 2 or 3) chimeric protein. In some embodiments, the cells are engineered to comprise 2, 3, 4, 5, 6, 7, 8, 9, 10, or more engineered nucleic acids, each encoding a promoter operably linked to a nucleotide sequence encoding at least one (e.g., 1, 2 or 3) chimeric protein. Engineered cells can comprise an engineered nucleic acid encoding at least one of the linkers described above, such as polypeptides that link a first polypeptide sequence and a second polypeptide sequence, one or more multicistronic linker described above, one or more additional promoters operably linked to additional ORFs, or a combination thereof.

In some embodiments, a cell (e.g., an immune cell or a stem cell) is engineered to express a protease. In some embodiments, a cell is engineered to express a protease heterologous to a cell. In some embodiments, a cell is engineered to express a protease heterologous to a cell expressing a chimeric protein, such as a heterologous protease that cleaves the protease cleavage site of a membrane-cleavable chimeric protein. In some embodiments, engineered cells comprise one or more engineered nucleic acids encoding a promoter operably linked to a nucleotide sequence encoding a protease, such as a heterologous protease. Protease and protease cleavage sites are described in greater detail in the Section herein titled “Protease Cleavage site.”

In some embodiments, a cell (e.g., an immune cell or a stem cell) is engineered to produce at least one homing molecule. “Homing,” refers to active navigation (migration) of a cell to a target site (e.g., a cell, tissue (e.g., tumor), or organ). A “homing molecule” refers to a molecule that directs cells to a target site. In some embodiments, a homing molecule functions to recognize and/or initiate interaction of an engineered cell to a target site. Non-limiting examples of homing molecules include CXCR1, CCR9, CXCR2, CXCR3, CXCR4, CCR2, CCR4, FPR2, VEGFR, IL6R, CXCR1, CSCR7, and PDGFR.

In some embodiments, a homing molecule is a chemokine receptor (cell surface molecule that binds to a chemokine). Non-limiting examples of chemokine receptors that may be produced by the engineered cells of the present disclosure include: CXC chemokine receptors (e.g., CXCR1, CXCR2, CXCR3, CXCR4, CXCR5, CXCR6, and CXCR7), CC chemokine receptors (CCR1, CCR2, CCR3, CCR4, CCR5, CCR6, CCR7, CCR8, CCR9, CCR10, and CCR11), CX3C chemokine receptors (e.g., CX3CR1, which binds to CX3CL1), and XC chemokine receptors (e.g., XCR1). In some embodiments, a chemokine receptor is a G protein-linked transmembrane receptor, or a member of the tumor necrosis factor (TNF) receptor superfamily (including but not limited to TNFRSF1A, TNFRSF1B). In some embodiments, cells are engineered to produce CXCL8, CXCL9, and/or CXCL10 (promote T-cell recruitment), CCL3 and/or CXCL5, CCL21 (Th1 recruitment and polarization). In some embodiments, cells are engineered to produce CXCR4.

In some embodiments, a cell (e.g., an immune cell or a stem cell) is engineered to produce G-protein coupled receptors (GPCRs) that detect N-formylated-containing oligopeptides (including but not limited to FPR2 and FPRL1).

In some embodiments, a cell (e.g., an immune cell or a stem cell) is engineered to produce receptors that detect interleukins (including but not limited to IL6R).

In some embodiments, a cell (e.g., an immune cell or a stem cell) is engineered to produce receptors that detect growth factors secreted from other cells, tissues, or tumors (including but not limited to FGFR, PDGFR, EGFR, and receptors of the VEGF family, including but not limited to VEGF-C and VEGF-D).

In some embodiments, a cell (e.g., an immune cell or a stem cell) is engineered to produce one or more integrins. Cells of the present disclosure may be engineered to produce any combination of integrin α and β subunits. The a subunit of an integrin may be, without limitation: ITGA1, ITGA2, ITGA3, ITGA4, ITGA5, ITGA6, IGTA7, ITGA8, ITGA9, IGTA10, IGTA11, ITGAD, ITGAE, ITGAL, ITGAM, ITGAV, ITGA2B, ITGAX. The β subunit of an integrin may be, without limitation: ITGB1, ITGB2, ITGB3, ITGB4, ITGB5, ITGB6, ITGB7, and ITGB8.

In some embodiments, a cell (e.g., an immune cell or a stem cell) is engineered to produce one or more matrix metalloproteinases (MMP). Non-limiting examples of MMPs include MMP-2, MMP-9, and MMP. In some embodiments, cells are engineered to produce an inhibitor of a molecule (e.g., protein) that inhibits MMPs. For example, cells may be engineered to express an inhibitor (e.g., an RNAi molecule) of membrane type 1 MMP (MT1-MMP) or TIMP metallopeptidase inhibitor 1 (TIMP-1).

In some embodiments, a cell (e.g., an immune cell or a stem cell) is engineered to produce a ligand that binds to selectin (e.g., hematopoietic cell E-/L-selectin ligand (HCELL), Dykstran et al., Stem Cells. 2016 October; 34 (10): 2501-2511) on the endothelium of a target tissue, for example.

The term “homing molecule” also encompasses transcription factors that regulate the production of molecules that improve/enhance homing of cells.

Also provided herein are engineered cells that are engineered to produce multiple chimeric proteins, at least two of which include effector molecules that modulate different tumor-mediated immunosuppressive mechanisms. In some embodiments, at least one (e.g., 1, 2, 3, 4, 5, or more) chimeric protein includes an effector molecule that stimulates at least one immunostimulatory mechanism in the tumor microenvironment, or inhibits at least one immunosuppressive mechanism in the tumor microenvironment. In some embodiments, at least one (e.g., 1, 2, 3, 4, 5, or more) chimeric protein includes an effector molecule that inhibits at least one immunosuppressive mechanism in the tumor microenvironment, and at least one chimeric protein (e.g., 1, 2, 3, 4, 5, or more) inhibits at least one immunosuppressive mechanism in the tumor microenvironment. In yet other embodiments, at least two (e.g., 2, 3, 4, 5, or more) chimeric proteins includes an effector molecule that stimulate at least one immunostimulatory mechanism in the tumor microenvironment. In still other embodiments, at least two (e.g., 1, 2, 3, 4, 5, or more) chimeric proteins includes an effector molecule that inhibit at least one immunosuppressive mechanism in the tumor microenvironment.

In some embodiments, a cell (e.g., an immune cell or a stem cell) is engineered to produce at least one chimeric protein including an effector molecule that stimulates T cell signaling, activity and/or recruitment. In some embodiments, a cell is engineered to produce at least one chimeric protein that includes an effector molecule that stimulates antigen presentation and/or processing. In some embodiments, a cell is engineered to produce at least one chimeric protein that includes an effector molecule that stimulates natural killer cell-mediated cytotoxic signaling, activity and/or recruitment. In some embodiments, a cell is engineered to produce at least one chimeric protein that includes an effector molecule that stimulates dendritic cell differentiation and/or maturation. In some embodiments, a cell is engineered to produce at least one chimeric protein that includes an effector molecule that stimulates immune cell recruitment. In some embodiments, a cell is engineered to produce at least one chimeric protein includes an effector molecule that that stimulates M1 macrophage signaling, activity and/or recruitment. In some embodiments, a cell is engineered to produce at least one chimeric protein that includes an effector molecule that stimulates Th1 polarization. In some embodiments, a cell is engineered to produce at least one chimeric protein that includes an effector molecule that stimulates stroma degradation. In some embodiments, a cell is engineered to produce at least one chimeric protein that includes an effector molecule that stimulates immunostimulatory metabolite production. In some embodiments, a cell is engineered to produce at least one chimeric protein that includes an effector molecule that stimulates Type I interferon signaling. In some embodiments, a cell is engineered to produce at least one chimeric protein that includes an effector molecule that inhibits negative costimulatory signaling. In some embodiments, a cell is engineered to produce at least one chimeric protein that includes an effector molecule that inhibits pro-apoptotic signaling (e.g., via TRAIL) of anti-tumor immune cells. In some embodiments, a cell is engineered to produce at least one chimeric protein that includes an effector molecule that inhibits T regulatory (T_{reg}) cell signaling, activity and/or recruitment. In some embodiments, a cell is engineered to produce at least one chimeric protein that includes an effector molecule that inhibits tumor checkpoint molecules. In some embodiments, a cell is engineered to produce at least one chimeric protein that includes an effector molecule that activates stimulator of interferon genes (STING) signaling. In some embodiments, a cell is engineered to produce at least one chimeric protein that includes an effector molecule that inhibits myeloid-derived suppressor cell signaling, activity and/or recruitment. In some embodiments, a cell is engineered to produce at least one chimeric protein that includes an effector molecule that degrades immunosuppressive factors/metabolites. In some embodiments, a cell is engineered to produce at least one chimeric protein that includes an effector molecule that inhibits vascular endothelial growth factor signaling. In some embodiments, a cell is engineered to produce at least one chimeric protein that includes an effector molecule that directly kills tumor cells (e.g., granzyme, perforin, oncolytic viruses, cytolytic peptides and enzymes, anti-tumor antibodies, e.g., that trigger ADCC).

In some embodiments, at least one chimeric protein including an effector molecule that: stimulates T cell signaling, activity and/or recruitment, stimulates antigen presentation and/or processing, stimulates natural killer cell-mediated cytotoxic signaling, activity and/or recruitment, stimulates dendritic cell differentiation and/or maturation, stimulates immune cell recruitment, stimulates macrophage signaling, stimulates stroma degradation, stimulates immunostimulatory metabolite production, or stimulates Type I interferon signaling; and at least one chimeric protein including an effector molecule that inhibits negative costimulatory signaling, inhibits pro-apoptotic signaling of anti-tumor immune cells, inhibits T regulatory (Treg) cell signaling, activity and/or recruitment, inhibits tumor checkpoint molecules, activates stimulator of interferon genes (STING) signaling, inhibits myeloid-derived suppressor cell signaling, activity and/or recruitment, degrades immunosuppressive factors/metabolites, inhibits vascular endothelial growth factor signaling, or directly kills tumor cells.

In some embodiments, a cell (e.g., an immune cell or a stem cell) is engineered to produce at least one chimeric protein including an effector molecule selected from IL-12, IFN-β, IFN-γ, IL-2, IL-15, IL-7, IL-36γ, IL-18, IL-1β, OX40-ligand, and CD40L; and/or at least a checkpoint inhibitor. Illustrative immune checkpoint molecules that can be targeted for blocking or inhibition include, but are not limited to, CTLA-4, 4-1BB (CD137), 4-1BBL (CD137L), PDL1, PDL2, PD1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, □□□□, and memory CD8+ (□□) T cells), CD160 (also referred to as BY55), and CGEN-15049. Immune checkpoint inhibitors include antibodies, or antigen binding fragments thereof, or other binding proteins, that bind to and block or inhibit the activity of one or more of CTLA-4, PDL1, PDL2, PD1, B7-H3, B7-H4, BTLA, HVEM, TIM3, GAL9, LAG3, TIM3, B7H3, B7H4, VISTA, KIR, 2B4, CD160, and CGEN-15049. Exemplary checkpoint inhibitors include, but are not limited to, anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GAL9 antibodies, anti-A2AR antibodies, anti-phosphatidylserine antibodies, anti-CD27 antibodies, anti-TNFa antibodies, anti-TREM1 antibodies, and anti-TREM2 antibodies. Illustrative immune checkpoint inhibitors include pembrolizumab (anti-PD-1; MK-3475/Keytruda®-Merck), nivolumamb (anti-PD-1; Opdivo®-BMS), pidilizumab (anti-PD-1 antibody; CT-011-Teva/CureTech), AMP224 (anti-PD-1; NCI), avelumab (anti-PD-L1; Bavencio®-Pfizer), durvalumab (anti-PD-L1; MEDI4736/Imfinzi®-Medimmune/AstraZeneca), atezolizumab (anti-PD-L1; Tecentriq®-Roche/Genentech), BMS-936559 (anti-PD-L1-BMS), tremelimumab (anti-CTLA-4; Medimmune/AstraZeneca), ipilimumab (anti-CTLA-4; Yervoy®-BMS), lirilumab (anti-KIR; BMS), monalizumab (anti-NKG2A; Innate Pharma/AstraZeneca).

In some embodiments, a cell (e.g., an immune cell or a stem cell) is engineered to produce at least one chimeric protein including an effector molecule selected from IL-12, IFN-β, IFN-γ, IL-2, IL-15, IL-7, IL-36γ, IL-18, IL-1β, OX40-ligand, and CD40L; and/or at least one checkpoint inhibitor selected from anti-PD-1 antibodies, anti-PD-L1 antibodies, anti-CTLA-4 antibodies, and anti-IL-35 antibodies; and/or at least one chimeric protein including an effector molecule selected from MIP1α (CCL3), MIP1β (CCL5), and CCL21; and/or at least one chimeric protein including an effector molecule selected from CpG oligodeoxynucleotides; and/or at least one chimeric protein including an effector molecule selected from microbial peptides.

In some embodiments, a cell (e.g., an immune cell or a stem cell) is engineered to produce IFN-β and at least one chimeric protein including an effector molecule selected from cytokines, antibodies, chemokines, nucleotides, peptides, enzymes, and stimulators of interferon genes (STINGs). In some embodiments, a cell is engineered to produce IFN-β and at least one cytokine or receptor/ligand (e.g., IL-12, IFN-γ, IL-2, IL-15, IL-7, IL-36γ, IL-18, IL-1β, OX40-ligand, and/or CD40L).

In some embodiments, a cell (e.g., an immune cell or a stem cell) is engineered to produce at least one membrane-cleavable chimeric protein where the secretable effector molecule (e.g., “S” in the formula S—C-MT or MT-C—S) is a cytokine, a chemokine, a homing molecule, a growth factor, a co-activation molecule, a tumor microenvironment modifier, a ligand, an antibody, a peptide, or an enzyme. In some embodiments, a cell is engineered to produce at least one membrane-cleavable chimeric protein where the secretable effector molecule is a cytokine. In some embodiments, a cell is engineered to produce at least one membrane-cleavable chimeric protein where the secretable effector molecule is a chemokine. In some embodiments, a cell is engineered to produce at least one membrane-cleavable chimeric protein where the secretable effector molecule is a homing molecule. In some embodiments, a cell is engineered to produce at least one membrane-cleavable chimeric protein where the secretable effector molecule is a growth factor. In some embodiments, a cell is engineered to produce at least one membrane-cleavable chimeric protein where the secretable effector molecule is a co-activation molecule. In some embodiments, a cell is engineered to produce at least one membrane-cleavable chimeric protein where the secretable effector molecule is a tumor microenvironment modifier. In some embodiments, a cell is engineered to produce at least one membrane-cleavable chimeric protein where the secretable effector molecule is a ligand. In some embodiments, a cell is engineered to produce at least one membrane-cleavable chimeric protein where the secretable effector molecule is an antibody. In some embodiments, a cell is engineered to produce at least one membrane-cleavable chimeric protein where the secretable effector molecule is a peptide. In some embodiments, a cell is engineered to produce at least one membrane-cleavable chimeric protein where the secretable effector molecule is an enzyme.

In some embodiments, a cell is engineered to produce at least one membrane-cleavable chimeric protein where the secretable effector molecule (e.g., “S” in the formula S—C-MT or MT-C—S) is IL-1-beta, IL-2, IL-4, IL-6, IL-7, IL-10, IL-12, an IL-12p70 fusion protein, IL-15, IL17A, IL18, IL21, IL22, Type I interferons, Interferon-gamma, or TNF-alpha. In some embodiments, a cell is engineered to produce at least one membrane-cleavable chimeric protein where the secretable effector molecule is CCL21a, CXCL10, CXCL11, CXCL13, a CXCL10-CXCL11 fusion protein, CCL19, CXCL9, or XCL1. In some embodiments, a cell is engineered to produce at least one membrane-cleavable chimeric protein where the secretable effector molecule is anti-integrin alpha4, beta7, anti-MAdCAM, SDF1, or MMP-2. In some embodiments, a cell is engineered to produce at least one membrane-cleavable chimeric protein where the secretable effector molecule is 4-1BBL or CD40L. In some embodiments, a cell is engineered to produce at least one membrane-cleavable chimeric protein where the secretable effector molecule is adenosine deaminase, a TGFbeta inhibitor, an immune checkpoint inhibitor, a VEGF inhibitor, or HPGE2. In some embodiments, a cell is engineered to produce at least one membrane-cleavable chimeric protein where the secretable effector molecule is an anti-TGFbeta peptide, an anti-TGFbeta antibody, a TGFb-TRAP, or combinations thereof. In some embodiments, a cell is engineered to produce at least one membrane-cleavable chimeric protein where the secretable effector molecule is an anti-VEGF antibody, an anti-VEGF peptide, or a combination thereof. In some embodiments, a cell is engineered to produce at least one membrane-cleavable chimeric protein where the secretable effector molecule is an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-KIR antibody, an anti-B7-H3 antibody, an anti-B7-H4 antibody, an anti-HVEM antibody, an anti-BTLA antibody, an anti-GAL9 antibody, an anti-A2AR antibody, an anti-phosphatidylscrine antibody, an anti-CD27 antibody, an anti-TNFa antibody, an anti-TREM1 antibody, or an anti-TREM2 antibody.

In some embodiments, a cell is engineered to produce at least one membrane-cleavable chimeric protein where the secretable effector molecule (e.g., “S” in the formula S—C-MT or MT-C—S) comprises IL-15. In some embodiments, a cell is engineered to produce at least one membrane-cleavable chimeric protein where the secretable effector molecule is a fusion of IL-15 and the sushi domain of IL-15Rα. In some embodiments, a cell is engineered to produce at least one membrane-cleavable chimeric protein where the secretable effector consists of IL-15. In some embodiments, a cell is engineered to produce at least one membrane-cleavable chimeric protein where the secretable effector molecule is IL-15 and the cell is further engineered to produce one or more additional effector molecules. In some embodiments, a cell is engineered to produce at least one membrane-cleavable chimeric protein where the secretable effector molecule is IL-15 and the cell is further engineered to produce one or more additional secretable effector molecules. In some embodiments, a cell is engineered to produce at least one membrane-cleavable chimeric protein where the secretable effector molecule is IL-15 and the cell is further engineered to produce a cytokine, a chemokine, a homing molecule, a growth factor, a co-activation molecule, a tumor microenvironment modifier, a ligand, an antibody, a polynucleotide, a peptide, or an enzyme. In some embodiments, a cell is engineered to produce at least one membrane-cleavable chimeric protein where the secretable effector molecule is IL-15 and the cell is further engineered to produce IL-12, IFN-γ, IL-2, IL-7, IL-36γ, IL-18, IL-1β, OX40-ligand, or CD40L. In some embodiments, a cell is engineered to produce at least one membrane-cleavable chimeric protein where the secretable effector molecule is IL-15 and the cell is further engineered to produce IL-12. In some embodiments, a cell is engineered to produce at least one membrane-cleavable chimeric protein where the secretable effector molecule is IL-15 and the cell is further engineered to produce IFN-γ. In some embodiments, a cell is engineered to produce at least one membrane-cleavable chimeric protein where the secretable effector molecule is IL-15 and the cell is further engineered to produce IL-2. In some embodiments, a cell is engineered to produce at least one membrane-cleavable chimeric protein where the secretable effector molecule is IL-15 and the cell is further engineered to produce IL-7. In some embodiments, a cell is engineered to produce at least one membrane-cleavable chimeric protein where the secretable effector molecule is IL-15 and the cell is further engineered to produce IL-36γ. In some embodiments, a cell is engineered to produce at least one membrane-cleavable chimeric protein where the secretable effector molecule is IL-15 and the cell is further engineered to produce IL-18. In some embodiments, a cell is engineered to produce at least one membrane-cleavable chimeric protein where the secretable effector molecule is IL-15 and the cell is further engineered to produce IL-1β. In some embodiments, a cell is engineered to produce at least one membrane-cleavable chimeric protein where the secretable effector molecule is IL-15 and the cell is further engineered to produce OX40-ligand. In some embodiments, a cell is engineered to produce at least one membrane-cleavable chimeric protein where the secretable effector molecule is IL-15 and the cell is further engineered to produce CD40L.

In some embodiments, a cell is engineered to produce at least one membrane-cleavable chimeric protein and the cell is further engineered to produce one or more additional membrane-cleavable chimeric proteins.

In some embodiments, a cell is engineered to produce at least one membrane-cleavable chimeric protein where the secretable effector molecule (e.g., “S” in the formula S—C-MT or MT-C—S) is IL-15 and the cell is further engineered to produce one or more additional membrane-cleavable chimeric proteins. In some embodiments, a cell is engineered to produce at least one membrane-cleavable chimeric protein where the secretable effector molecule is IL-15 and the cell is further engineered an additional membrane-cleavable chimeric protein where the additional secretable effector molecule is produce a cytokine, a chemokine, a homing molecule, a growth factor, a co-activation molecule, a tumor microenvironment modifier, a ligand, an antibody, a polynucleotide, a peptide, or an enzyme. In some embodiments, a cell is engineered to produce at least one membrane-cleavable chimeric protein where the secretable effector molecule is IL-15 and the cell is further engineered an additional membrane-cleavable chimeric protein where the additional secretable effector molecule is IL-12, IFN-γ, IL-2, IL-7, IL-36γ, IL-18, IL-1β, OX40-ligand, or CD40L. In some embodiments, a cell is engineered to produce at least one membrane-cleavable chimeric protein where the secretable effector molecule is IL-15 and the cell is further engineered an additional membrane-cleavable chimeric protein where the additional secretable effector molecule is IL-12. In some embodiments, a cell is engineered to produce at least one membrane-cleavable chimeric protein where the secretable effector molecule is IL-15 and the cell is further engineered an additional membrane-cleavable chimeric protein where the additional secretable effector molecule is IFN-γ. In some embodiments, a cell is engineered to produce at least one membrane-cleavable chimeric protein where the secretable effector molecule is IL-15 and the cell is further engineered an additional membrane-cleavable chimeric protein where the additional secretable effector molecule is IL-2. In some embodiments, a cell is engineered to produce at least one membrane-cleavable chimeric protein where the secretable effector molecule is IL-15 and the cell is further engineered an additional membrane-cleavable chimeric protein where the additional secretable effector molecule is IL-7. In some embodiments, a cell is engineered to produce at least one membrane-cleavable chimeric protein where the secretable effector molecule is IL-15 and the cell is further engineered an additional membrane-cleavable chimeric protein where the additional secretable effector molecule is IL-36γ. In some embodiments, a cell is engineered to produce at least one membrane-cleavable chimeric protein where the secretable effector molecule is IL-15 and the cell is further engineered an additional membrane-cleavable chimeric protein where the additional secretable effector molecule is IL-18. In some embodiments, a cell is engineered to produce at least one membrane-cleavable chimeric protein where the secretable effector molecule is IL-15 and the cell is further engineered an additional membrane-cleavable chimeric protein where the additional secretable effector molecule is IL-1β. In some embodiments, a cell is engineered to produce at least one membrane-cleavable chimeric protein where the secretable effector molecule is IL-15 and the cell is further engineered an additional membrane-cleavable chimeric protein where the additional secretable effector molecule is OX40-ligand. In some embodiments, a cell is engineered to produce at least one membrane-cleavable chimeric protein where the secretable effector molecule is IL-15 and the cell is further engineered an additional membrane-cleavable chimeric protein where the additional secretable effector molecule is CD40L.

A cell can also be further engineered to express additional proteins in addition to the chimeric proteins (e.g., the membrane-cleavable chimeric proteins having the formula S—C-MT or MT-C—S described herein), proteins of interest, or effector molecules described herein. A cell can be further engineered to express one or more antigen recognizing receptors. Examples of antigens that may be targeted by one or more antigen recognizing receptors include, but are not limited to, 5T4, ADAM9, AFP, AXL, B7-H3, B7-H4, B7-H6, BCMA, C4.4, CA6, Cadherin 3, Cadherin 6, CCR4, CD19, CD20, CD22, CD123, CD133, CD138, CD142, CD166, CD25, CD30, CD33, CD352, CD37, CD38, CD44, CD56, CD66c, CD70, CD71, CD74, CD79b, CD80, CEA, CEACAM5, Claudin 18.2, cMet, CSPG4, CTLA, DLK1, DLL3, DR5, EGFR, ENPP3, EpCAM, EphA2, Ephrin A4, ETBR, FGFR2, FGFR3, FLT3, FRalpha, FRb, GCC, GD2, GFRa4, gpA33, GPC2, GPC3, gpNBM, GPRC5, HER2, IL-13R, IL-13Ra, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUCIC, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.

An antigen recognizing receptor can include an antigen-binding domain, such as an antibody, an antigen-binding fragment of an antibody, a F(ab) fragment, a F(ab′) fragment, a single chain variable fragment (scFv), or a single-domain antibody (sdAb). An antigen recognizing receptors can include an scFv. An scFv can include a heavy chain variable domain (VH) and a light chain variable domain (VL), which can be separated by a peptide linker. For example, an scFv can include the structure VH-L-VL or VL-L-VH, wherein VH is the heavy chain variable domain, L is the peptide linker, and VL is the light chain variable domain.

An antigen recognizing receptor can be a chimeric antigen receptor (CAR). A CAR can have one or more intracellular signaling domains, such as a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, fragments thereof, combinations thereof, or combinations of fragments thereof. A CAR can have a transmembrane domain, such as a CD8 transmembrane domain, a CD28 transmembrane domain a CD3zeta-chain transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, a BTLA transmembrane domain, fragments thereof, combinations thereof, or combinations of fragments thereof. A CAR can have a spacer region between the antigen-binding domain and the transmembrane domain.

An antigen recognizing receptor can be a T cell receptor (TCR).

### Engineered Cell Types

Also provided herein are engineered cells. Cells can be engineered to comprise any of the engineered nucleic acids described herein (e.g., any of the multicistronic systems encoding each of the membrane-cleavable chimeric proteins, the bivalent aCARs, and the iCARs described herein). Cells can be engineered to possess any of the features of any of the engineered cells described herein. In a particular aspect, provided herein are cells engineered to produce each of (i) a membrane-cleavable chimeric protein; (ii) a bivalent aCAR; and (iii) an iCAR. In a particular aspect, provided herein are cells engineered to produce two or more chimeric proteins. In a particular aspect, provided herein are cells engineered to produce two or more of the chimeric proteins described herein, where each chimeric protein is a different protein of interest or effector molecule. In a particular aspect, provided herein are cells engineered to produce any of the chimeric proteins described herein and engineered to separately produce a different effector molecule or protein of interest (e.g., a homing molecule, antigen receptor, etc.).

The engineered cells can be an immune cell, including but not limited to, a T cell, a CD8+ T cell, a CD4+ T cell, a gamma-delta (γδ) T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, or a dendritic cell. The engineered cells can be a T cell. The engineered cells can be an NK cell. Cells engineered to express a chimeric antigen receptor (CAR) are also referred to as CAR cells. For example, T cells engineered to express a CAR are also referred to as CAR-T cells and likewise NK cells engineered to express a CAR are also referred to as CAR-NK cells.

The engineered cells can be a stem cell, including but not limited to, a human embryonic stem cell (ESC), an ESC-derived cell, a pluripotent stem cell, a mesenchymal stromal cell (MSC), an induced pluripotent stem cell (iPSC), or an iPSC-derived cell.

The engineered cells can be tumor-derived cells. Examples of tumor cells include, but are not limited to, a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, an ovarian tumor cell, a pancreatic tumor cell, a prostate tumor cell, a skin tumor cell, a thyroid tumor cell, and a uterine tumor cell.

A cell can be engineered to produce the chimeric proteins using methods known to those skilled in the art. For example, cells can be transduced to engineer the tumor. In an embodiment, the cell is transduced using a virus.

In a particular embodiment, the cell is transduced using an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof.

The virus, including any of the oncolytic viruses described herein, can be a recombinant virus that encodes one more transgenes encoding one or more chimeric proteins, such as any of the engineered nucleic acids described herein. The virus, including any of the oncolytic viruses described herein, can be a recombinant virus that encodes one more transgenes encoding one or more of the two or more chimeric proteins, such as any of the engineered nucleic acids described herein.

Also provided herein are engineered erythrocytes. Erythrocytes can be engineered to comprise any of the engineered nucleic acids described herein. Erythrocytes can be engineered to possess any of the features of any of the engineered cells described herein. In a particular aspect, provided herein are erythrocytes engineered to produce one or more of the chimeric proteins described herein. In a particular aspect, provided herein are erythrocytes engineered to produce two or more of the chimeric proteins described herein.

Also provided herein are engineered platelet cells. Platelet cells can be engineered to comprise any of the engineered nucleic acids described herein. Platelet cells can be engineered to possess any of the features of any of the engineered cells described herein. In a particular aspect, provided herein are platelet cells engineered to produce one or more of the chimeric proteins described herein. In a particular aspect, provided herein are platelet cells engineered to produce two or more of the chimeric proteins described herein.

Also provided herein are engineered bacterial cells. Bacterial cells can be engineered to comprise any of the engineered nucleic acids described herein. Bacterial cells can be engineered to possess any of the features of any of the engineered cells described herein. In a particular aspect, provided herein are bacterial cells engineered to produce two or more of the chimeric proteins described herein. Bacterial cells can be engineered to produce one or more mammalian-derived chimeric proteins. Bacterial cells can be engineered to produce two or more mammalian-derived chimeric proteins. Examples of bacterial cells include, but are not limited to, Clostridium beijerinckii, Clostridium sporogenes, Clostridium novyi, Escherichia coli, Pseudomonas aeruginosa, Listeria monocytogenes, Salmonella typhimurium, and Salmonella choleraesuis.

An engineered cell can be a human cell. An engineered cell can be a human primary cell. An engineered primary cell can be a tumor infiltrating primary cell. An engineered primary cell can be a primary T cell. An engineered primary cell can be a hematopoietic stem cell (HSC). An engineered primary cell can be a natural killer (NK) cell. An engineered primary cell can be any somatic cell. An engineered primary cell can be a MSC. Human cells (e.g., immune cells) can be engineered to comprise any of the engineered nucleic acids described herein. Human cells (e.g., immune cells) can be engineered to possess any of the features of any of the engineered cells described herein. In a particular aspect, provided herein are human cells (e.g., immune cells) engineered to produce one or more of the chimeric proteins described herein. In a particular aspect, provided herein are human cells (e.g., immune cells) engineered to produce two or more of the chimeric proteins described herein.

An engineered cell can be isolated from a subject (autologous), such as a subject known or suspected to have cancer. Cell isolation methods are known to those skilled in the art and include, but are not limited to, sorting techniques based on cell-surface marker expression, such as FACS sorting, positive isolation techniques, and negative isolation, magnetic isolation, and combinations thereof. An engineered cell can be allogenic with reference to the subject being administered a treatment. Allogenic modified cells can be HLA-matched to the subject being administered a treatment. An engineered cell can be a cultured cell, such as an ex vivo cultured cell. An engineered cell can be an ex vivo cultured cell, such as a primary cell isolated from a subject. A cultured cell can be cultured with one or more cytokines.

Also provided herein are methods that include culturing the engineered cells of the present disclosure. Methods of culturing the engineered cells described herein are known. One skilled in the art will recognize that culturing conditions will depend on the particular engineered cell of interest. One skilled in the art will recognize that culturing conditions will depend on the specific downstream use of the engineered cell, for example, specific culturing conditions for subsequent administration of the engineered cell to a subject.

### Methods of Engineering Cells

Also provided herein are compositions and methods for engineering cells to produce one or more proteins of interest or effector molecules (e.g., the multicistronic expression systems that encode each of (i) a membrane-cleavable chimeric protein; (ii) a bivalent aCAR; and (iii) an iCAR described herein).

In general, cells are engineered to produce proteins of interest or effector molecules through introduction (i.e., delivery) of polynucleotides encoding the one or more proteins of interest or effector molecules, e.g., the chimeric proteins described herein including the protein of interest or effector molecule, into the cell's cytosol and/or nucleus. For example, the polynucleotides encoding the one or more chimeric proteins can be any of the multicistronic expression systems that encode each of (i) a membrane-cleavable chimeric protein; (ii) a bivalent aCAR; and (iii) an iCAR described herein. Delivery methods include, but are not limited to, viral-mediated delivery, lipid-mediated transfection, nanoparticle delivery, electroporation, sonication, and cell membrane deformation by physical means. One skilled in the art will appreciate the choice of delivery method can depend on the specific cell type to be engineered.

### Viral-Mediated Delivery

Viral vector-based delivery platforms can be used to engineer cells. In general, a viral vector-based delivery platform engineers a cell through introducing (i.e., delivering) into a host cell. For example, a viral vector-based delivery platform can engineer a cell through introducing any of the engineered nucleic acids described herein (e.g., any of the multicistronic systems encoding each of each of (i) a membrane-cleavable chimeric protein; (ii) a bivalent aCAR; and (iii) an iCAR described herein, and/or any of the expression cassettes described herein containing a promoter and an exogenous polynucleotide sequence encoding the chimeric proteins, oriented from N-terminal to C-terminal). A viral vector-based delivery platform can be a nucleic acid, and as such, an engineered nucleic acid can also encompass an engineered virally-derived nucleic acid. Such engineered virally-derived nucleic acids can also be referred to as recombinant viruses or engineered viruses.

A viral vector-based delivery platform can encode more than one engineered nucleic acid, gene, or transgene within the same nucleic acid. For example, an engineered virally-derived nucleic acid, e.g., a recombinant virus or an engineered virus, can encode one or more transgenes, including, but not limited to, any of the engineered nucleic acids described herein that encode one or more of the chimeric proteins described herein. The one or more transgenes encoding the one or more chimeric proteins can be configured to express the one or more chimeric proteins and/or other protein of interest. A viral vector-based delivery platform can encode one or more genes in addition to the one or more transgenes (e.g., transgenes encoding the one or more chimeric proteins and/or other protein of interest), such as viral genes needed for viral infectivity and/or viral production (e.g., capsid proteins, envelope proteins, viral polymerases, viral transcriptases, etc.), referred to as cis-acting elements or genes.

A viral vector-based delivery platform can comprise more than one viral vector, such as separate viral vectors encoding the engineered nucleic acids, genes, or transgenes described herein, and referred to as trans-acting elements or genes. For example, a helper-dependent viral vector-based delivery platform can provide additional genes needed for viral infectivity and/or viral production on one or more additional separate vectors in addition to the vector encoding the one or more chimeric proteins and/or other protein of interest. One viral vector can deliver more than one engineered nucleic acids, such as one vector that delivers engineered nucleic acids that are configured to produce two or more chimeric proteins and/or other protein of interest. More than one viral vector can deliver more than one engineered nucleic acids, such as more than one vector that delivers one or more engineered nucleic acid configured to produce one or more chimeric proteins and/or other protein of interest. The number of viral vectors used can depend on the packaging capacity of the above mentioned viral vector-based vaccine platforms, and one skilled in the art can select the appropriate number of viral vectors.

In general, any of the viral vector-based systems can be used for the in vitro production of molecules, such as the chimeric proteins, effector molecules, and/or other protein of interest described herein, or used in vivo and ex vivo gene therapy procedures, e.g., for in vivo delivery of the engineered nucleic acids encoding one or more chimeric proteins and/or other protein of interest. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can be RNA-based viruses or DNA-based viruses. Exemplary viral vector-based delivery platforms include, but are not limited to, a herpes simplex virus, a adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239 (1): 45-61, Sakuman et al., Lentiviral vectors: basic to translational, Biochem J. (2012) 443 (3): 603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

The sequences may be preceded with one or more sequences targeting a subcellular compartment. Upon introduction (i.e. delivery) into a host cell, infected cells (i.e., an engineered cell) can express the chimeric proteins and/or other protein of interest. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for the introduction (i.e., delivery) of engineered nucleic acids, e.g., Salmonella typhi vectors, and the like will be apparent to those skilled in the art from the description herein.

The viral vector-based delivery platforms can be a virus that targets a cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more transgenes (e.g., an engineered nucleic acid) encoding one or more chimeric proteins and/or other protein of interest. The transgenes encoding the one or more chimeric proteins and/or other protein of interest can be configured to express the chimeric proteins and/or other protein of interest.

The viral vector-based delivery platform can be retrovirus-based. In general, retroviral vectors are comprised of cis-acting long terminal repeats with packaging capacity for up to 6-10 kb of foreign sequence. The minimum cis-acting LTRs are sufficient for replication and packaging of the vectors, which are then used to integrate the one or more engineered nucleic acids (e.g., transgenes encoding the one or more chimeric proteins and/or other protein of interest) into the target cell to provide permanent transgene expression. Retroviral-based delivery systems include, but are not limited to, those based upon murine leukemia, virus (MuLV), gibbon ape leukemia virus (GaLV), Simian Immuno deficiency vims (SIV), human immuno deficiency vims (HIV), and combinations thereof (see, e.g., Buchscher et al., J. Virol. 66:2731-2739 (1992); Johann et ah, J. Virol. 66:1635-1640 (1992); Sommnerfelt et al., Virol. 176:58-59 (1990); Wilson et ah, J. Virol. 63:2374-2378 (1989); Miller et al, J, Virol. 65:2220-2224 (1991); PCT/US94/05700). Other retroviral systems include the Phoenix retrovirus system.

The viral vector-based delivery platform can be lentivirus-based. In general, lentiviral vectors are retroviral vectors that are able to transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

The viral vector-based delivery platform can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,585,362; 6,083,716, 7,371,570; 7,348,178; 7,323,177; 7,319,033; 7,318,919; and 7,306,793 and International Patent Application WO96/13597, each herein incorporated by reference for all purposes.

The viral vector-based delivery platform can be adeno-associated virus (AAV)-based. Adeno-associated virus (“AAV”) vectors may be used to transduce cells with engineered nucleic acids (e.g., any of the engineered nucleic acids described herein). AAV systems can be used for the in vitro production of proteins of interest, such as the chimeric proteins described herein and/or effector molecules, or used in vivo and ex vivo gene therapy procedures, e.g., for in vivo delivery of the engineered nucleic acids encoding one or more chimeric proteins and/or other protein of interest (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003-0138772, US 2007/0036760, and US 2009/0197338; Gao, et al., J. Virol, 78 (12): 6381-6388 (June 2004); Gao, et al, Proc Natl Acad Sci USA, 100 (10): 6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each herein incorporated by reference for all purposes). Exemplary methods for constructing recombinant AAV vectors are described in more detail in U.S. Pat. No. 5,173,414; Tratschin et ah, Mol. Cell. Biol. 5:3251-3260 (1985); Tratschin, et ah, Mol. Cell, Biol. 4:2072-2081 (1984); Hermonat &amp; Muzyczka, PNAS 81:64666470 (1984); and Samuiski et ah, J. Virol. 63:03822-3828 (1989), each herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof. In particular examples, an AAV-based vector has a capsid protein having an amino acid sequence corresponding to AAV2. In particular examples, an AAV-based vector has a capsid protein having an amino acid sequence corresponding to AAV8.

AAV vectors can be engineered to have any of the exogenous polynucleotide sequences encoding the membrane-cleavable chimeric proteins described herein having the formula: S—C-MT or MT-C—S.

The viral vector-based delivery platform can be a virus-like particle (VLP) platform. In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Scow et al. (Mol Ther. 2009 May; 17 (5): 767-777), herein incorporated by reference for all purposes.

The viral vector-based delivery platform can be engineered to target (i.e., infect) a range of cells, target a narrow subset of cells, or target a specific cell. In general, the envelope protein chosen for the viral vector-based delivery platform will determine the viral tropism. The virus used in the viral vector-based delivery platform can be pseudotyped to target a specific cell of interest. The viral vector-based delivery platform can be pantropic and infect a range of cells. For example, pantropic viral vector-based delivery platforms can include the VSV-G envelope. The viral vector-based delivery platform can be amphotropic and infect mammalian cells. Accordingly, one skilled in the art can select the appropriate tropism, pseudotype, and/or envelope protein for targeting a desired cell type.

### Lipid Structure Delivery Systems

Engineered nucleic acids (e.g., any of the engineered nucleic acids described herein) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the engineered nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., an engineered nucleic acid, such as any of the engineered nucleic acids described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers and the like. Liposomes can be unilamellar liposomes. Liposomes can be multilamellar liposomes. Liposomes can be multivesicular liposomes. Liposomes can be positively charged, negatively charged, or neutrally charged. In certain embodiments, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szokan et al., Ann. Rev. Biophys. Bioeng. 9; 467 (1980), U.S. Pat. Nos. 4,235,871, 4,501,728, 4,501,728, 4,837,028, and 5,019,369, each herein incorporated by reference for all purposes.

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, an engineered nucleic acid, such as any of the engineered nucleic acids described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with the lipid bilayer.

A liposome used according to the present embodiments can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes.

Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety.

Lipid-mediated gene delivery methods are described, for instance, in WO 96/18372; WO 93/24640; Mannino & Gould-Fogerite, BioTechniques 6 (7): 682-691 (1988); U.S. Pat. No. 5,279,833 Rose U.S. Pat. No. 5,279,833; WO91/06309; and Felgner et al., Proc. Natl. Acad. Sci. USA 84:7413-7414 (1987), each herein incorporated by reference for all purposes.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome, and exhibit membrane proteins from the parental cell on the surface. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any of the engineered nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

Lipid nanoparticles (LNPs), in general, are synthetic lipid structures that rely on the amphiphilic nature of lipids to form membranes and vesicle like structures (Riley 2017). In general, these vesicles deliver cargo/payloads, such as any of the engineered nucleic acids or viral systems described herein, by absorbing into the membrane of target cells and releasing the cargo into the cytosol. Lipids used in LNP formation can be cationic, anionic, or neutral. The lipids can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical synthetic lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25 (7): 1501-1513), herein incorporated by reference for all purposes.

Nucleic-acid vectors, such as expression vectors, exposed directly to serum can have several undesirable consequences, including degradation of the nucleic acid by serum nucleases or off-target stimulation of the immune system by the free nucleic acids. Similarly, viral delivery systems exposed directly to serum can trigger an undesired immune response and/or neutralization of the viral delivery system. Therefore, encapsulation of an engineered nucleic acid and/or viral delivery system can be used to avoid degradation, while also avoiding potential off-target affects. In certain examples, an engineered nucleic acid and/or viral delivery system is fully encapsulated within the delivery vehicle, such as within the aqueous interior of an LNP. Encapsulation of an engineered nucleic acid and/or viral delivery system within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with an engineered nucleic acid or viral delivery system and any other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the cargo/payload (e.g., an engineered nucleic acid and/or viral delivery system) can be further treated or engineered to prepare them for administration.

### Nanoparticle Delivery

Nanomaterials can be used to deliver engineered nucleic acids (e.g., any of the engineered nucleic acids described herein). Nanomaterial vehicles, importantly, can be made of non-immunogenic materials and generally avoid eliciting immunity to the delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery—A Review. Nanomaterials 2017, 7 (5), 94), herein incorporated by reference for all purposes.

### Genomic Editing Systems

A genomic editing systems can be used to engineer a host genome to encode an engineered nucleic acid, such as an engineered nucleic acid encoding one or more of the chimeric proteins (e.g., any of the multicistronic systems encoding each of (i) a membrane-cleavable chimeric protein; (ii) a bivalent aCAR; and (iii) an iCAR described herein). In general, a “genomic editing system” refers to any system for integrating an exogenous gene into a host cell's genome. Genomic editing systems include, but are not limited to, a transposon system, a nuclease genomic editing system, and a viral vector-based delivery platform.

A transposon system can be used to integrate an engineered nucleic acid, such as an engineered nucleic acid encoding one or more of the chimeric proteins (e.g., any of the multicistronic systems encoding each of (i) a membrane-cleavable chimeric protein; (ii) a bivalent aCAR; and (iii) an iCAR described herein), into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., an engineered nucleic acid) randomly into a host genome. Examples of transposon systems include systems using a transposon of the Tc1/mariner transposon superfamily, such as a Sleeping Beauty transposon system, described in more detail in Hudecek et al. (Crit Rev Biochem Mol Biol. 2017 August; 52 (4): 355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes.

A nuclease genomic editing system can be used to engineer a host genome to encode an engineered nucleic acid, such as an engineered nucleic acid encoding one or more of the chimeric proteins (e.g., any of the multicistronic systems encoding each of (i) a membrane-cleavable chimeric protein; (ii) a bivalent aCAR; and (iii) an iCAR described herein). Without wishing to be bound by theory, in general, the nuclease-mediated gene editing systems used to introduce an exogenous gene take advantage of a cell's natural DNA repair mechanisms, particularly homologous recombination (HR) repair pathways. Briefly, following an insult to genomic DNA (typically a double-stranded break), a cell can resolve the insult by using another DNA source that has identical, or substantially identical, sequences at both its 5′ and 3′ ends as a template during DNA synthesis to repair the lesion. In a natural context, HDR can use the other chromosome present in a cell as a template. In gene editing systems, exogenous polynucleotides are introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HDR. Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the engineered nucleic acids described herein, such as any of the engineered nucleic acids encoding one or more chimeric proteins (e.g., any of the multicistronic systems encoding each of each of (i) a membrane-cleavable chimeric protein; (ii) a bivalent aCAR; and (iii) an iCARdescribed herein)).

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to encode an engineered nucleic acid, such as an engineered nucleic acid encoding one or more of the chimeric proteins (e.g., any of the multicistronic systems encoding each of (i) a membrane-cleavable chimeric protein; (ii) a bivalent aCAR; and (iii) an iCAR described herein). CRISPR systems are described in more detail in M. Adli (“The CRISPR tool kit for genome editing and beyond” Nature Communications; volume 9 (2018), Article number: 1911), herein incorporated by reference for all that it teaches. In general, a CRISPR-mediated gene editing system comprises a CRISPR-associated (Cas) nuclease and a RNA(s) that directs cleavage to a particular target sequence. An exemplary CRISPR-mediated gene editing system is the CRISPR/Cas9 systems comprised of a Cas9 nuclease and a RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). While the Cas9 system is illustrated here, other CRISPR systems can be used, such as the Cpf1/Cas12 or Cas13 systems. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a T cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can be also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The engineered cells described herein can be engineered using non-viral methods, e.g., the nuclease and/or CRISPR mediated gene editing systems described herein can be delivered to a cell using non-viral methods. The engineered cells described herein can be engineered using viral methods, e.g., the nuclease and/or CRISPR mediated gene editing systems described herein can be delivered to a cell using viral methods such as adenoviral, retroviral, lentiviral, or any of the other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is an engineered site-specific nuclease, which is composed of the DNA-binding domain of TALE (transcription activator-like effectors) and the catalytic domain of restriction endonuclease Fokl. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

### Other Engineering Delivery Systems

Various additional means to introduce engineered nucleic acids (e.g., any of the engineered nucleic acids described herein) into a cell or other target recipient entity, such as any of the lipid structures described herein.

Electroporation can used to deliver polynucleotides to recipient entities. Electroporation is a method of internalizing a cargo/payload into a target cell or entity's interior compartment through applying an electrical field to transiently permeabilize the outer membrane or shell of the target cell or entity. In general, the method involves placing cells or target entities between two electrodes in a solution containing a cargo of interest (e.g., any of the engineered nucleic acids described herein). The lipid membrane of the cells is then disrupted, i.e., permeabilized, by applying a transient set voltage that allows the cargo to enter the interior of the entity, such as the cytoplasm of the cell. In the example of cells, at least some, if not a majority, of the cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, the type of cell or other recipient entity, the cargo to be delivered, the efficiency of internalization desired, and the viability desired. Optimization of such criteria are within the scope of those skilled in the art. A variety devices and protocols can be used for electroporation. Examples include, but are not limited to, Neon® Transfection System, MaxCyte® Flow Electroporation™, Lonza® Nucleofector™ systems, and Bio-Rad® electroporation systems.

Other means for introducing engineered nucleic acids (e.g., any of the engineered nucleic acids described herein) into a cell or other target recipient entity include, but are not limited to, sonication, gene gun, hydrodynamic injection, and cell membrane deformation by physical means.

Compositions and methods for delivering engineered mRNAs in vivo, such as naked plasmids or mRNA, are described in detail in Kowalski et al. (Mol Ther. 2019 Apr. 10; 27 (4): 710-728) and Kaczmarek et al. (Genome Med. 2017; 9:60), each herein incorporated by reference for all purposes.

### Delivery Vehicles

Also provided herein are compositions for delivering a cargo/payload (a “delivery vehicle”).

The cargo can comprise nucleic acids (e.g., any of the engineered nucleic acids described herein, such as any of the multicistronic systems encoding each of (i) a membrane-cleavable chimeric protein; (ii) a bivalent aCAR; and (iii) an iCAR described herein), as described above. The cargo can comprise proteins, carbohydrates, lipids, small molecules, and/or combinations thereof. The cargo can be any of the chimeric proteins provided for herein (e.g., any of the multicistronic systems encoding each of (i) a membrane-cleavable chimeric protein; (ii) a bivalent aCAR; and (iii) an iCAR described herein). The cargo can be a combination of the chimeric proteins described herein, e.g., two or more of the chimeric proteins described herein. The cargo can be a combination of the chimeric protein described herein and another cargo of interest, such as another protein, carbohydrate, lipid, small molecule, and/or combination thereof.

The delivery vehicle can comprise any composition suitable for delivering a cargo. The delivery vehicle can comprise any composition suitable for delivering a protein (e.g., any of the chimeric proteins described herein). The delivery vehicle can be any of the lipid structure delivery systems described herein. For example, a delivery vehicle can be a lipid-based structure including, but not limited to, a lipid-based nanoparticle, a liposome, a micelle, an exosome, a vesicle, an extracellular vesicle, a cell, or a tissue. The delivery vehicle can be any of the nanoparticles described herein, such as nanoparticles comprising lipids (as previously described), inorganic nanomaterials, and other polymeric materials.

The delivery vehicle can be capable of delivering the cargo to a cell, such as delivering any of the chimeric proteins described herein to a cell. The delivery vehicle can be capable of delivering the cargo to a cell, such as delivering any of the chimeric proteins described herein to a cell. The delivery vehicle can be configured to target a specific cell, such as configured with a re-directing antibody to target a specific cell. The delivery vehicle can be capable of delivering the cargo to a cell in vivo.

The delivery vehicle can be capable of delivering the cargo to a tissue or tissue environment (e.g., a tumor microenvironment), such as delivering any of the chimeric proteins described herein to a tissue or tissue environment in vivo. Delivering a cargo can include secreting the cargo, such as secreting any of the chimeric proteins described herein. Accordingly, the delivery vehicle can be capable of secreting the cargo, such as secreting any of the chimeric proteins described herein. The delivery vehicle can be capable of secreting the cargo to a tissue or tissue environment (e.g., a tumor microenvironment), such as secreting any of the chimeric proteins described herein into a tissue or tissue environment. The delivery vehicle can be configured to target a specific tissue or tissue environment (e.g., a tumor microenvironment), such as configured with a re-directing antibody to target a specific tissue or tissue environment.

### Methods of Treatment

Further provided herein are methods that include delivering, or administering, to a subject (e.g., a human subject) engineered cells as provided herein to produce in vivo at least one protein of interest produced by the engineered cells (e.g., any of the chimeric proteins provided for herein, such as any of the multicistronic systems encoding each of (i) a membrane-cleavable chimeric protein; (ii) a bivalent aCAR; and (iii) an iCAR described herein, or the secreted effector molecules provided for herein following protease cleavage of the chimeric protein). Further provided herein are methods that include delivering, or administering, to a subject (e.g., a human subject) engineered cells as provided herein to produce in vivo at least two proteins of interest, e.g., at least two of the chimeric proteins provided for herein, such as each of (i) a membrane-cleavable chimeric protein; (ii) a bivalent aCAR; and (iii) an iCAR described herein, produced by the engineered cells.

Further provided herein are methods that include delivering, or administering, to a subject (e.g., a human subject) any of the delivery vehicles described herein, such as any of the delivery vehicles described herein comprising any of the proteins of interest described herein, e.g., any of the chimeric proteins provided for herein, such as each of (i) a membrane-cleavable chimeric protein; (ii) a bivalent aCAR; and (iii) an iCAR described herein. Further provided herein are methods that include delivering, or administering, to a subject (e.g., a human subject) any of the delivery vehicles described herein, such as any of the delivery vehicles described herein comprising two or more proteins of, e.g., at least two of the chimeric proteins provided for herein, such as each of (i) a membrane-cleavable chimeric protein; (ii) a bivalent aCAR; and (iii) an iCAR described herein.

In some embodiments, the engineered cells or delivery vehicles are administered via intravenous, intraperitoneal, intratracheal, subcutaneous, intratumoral, oral, anal, intranasal (e.g., packed in a delivery particle), or arterial (e.g., internal carotid artery) routes. Thus, the engineered cells or delivery vehicles may be administered systemically or locally (e.g., to a TME or via intratumoral administration). An engineered cell can be isolated from a subject, such as a subject known or suspected to have cancer. An engineered cell can be allogenic with reference to the subject being administered a treatment. Allogenic modified cells can be HLA-matched to the subject being administered a treatment. Delivery vehicles can be any of the lipid structure delivery systems described herein. Delivery vehicles can be any of the nanoparticles described herein.

Engineered cells or delivery vehicles can be administered alone or in combination with other treatments, either simultaneously or sequentially dependent upon the condition to be treated. For example, engineered cells or delivery vehicles can be administered in combination with one or more IMiDs described herein. FDA-approved IMiDs can be administered in their approved fashion. In another example, engineered cells or delivery vehicles can be administered in combination with a checkpoint inhibitor therapy. Exemplary checkpoint inhibitors include, but are not limited to, anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GAL9 antibodies, anti-A2AR antibodies, anti-phosphatidylserine antibodies, anti-CD27 antibodies, anti-TNFa antibodies, anti-TREM1 antibodies, and anti-TREM2 antibodies. Illustrative immune checkpoint inhibitors include pembrolizumab (anti-PD-1; MK-3475/Keytruda®-Merck), nivolumamb (anti-PD-1; Opdivo®-BMS), pidilizumab (anti-PD-1 antibody; CT-011-Teva/CureTech), AMP224 (anti-PD-1; NCI), avelumab (anti-PD-L1; Bavencio®-Pfizer), durvalumab (anti-PD-L1; MEDI4736/Imfinzi®-Medimmune/AstraZeneca), atezolizumab (anti-PD-L1; Tecentriq®-Roche/Genentech), BMS-936559 (anti-PD-L1-BMS), tremelimumab (anti-CTLA-4; Medimmune/AstraZeneca), ipilimumab (anti-CTLA-4; Yervoy®-BMS), lirilumab (anti-KIR; BMS), monalizumab (anti-NKG2A; Innate Pharma/AstraZeneca). In other examples, engineered cells or delivery vehicles can be administered in combination with TGFbeta inhibitors, VEGF inhibitors, or HPGE2. In another example, engineered cells or delivery vehicles can be administered in combination with an anti-CD40 antibody.

Some methods comprise selecting a subject (or patient population) having a tumor (or cancer) and treating that subject with engineered cells or delivery vehicles that modulate tumor-mediated immunosuppressive mechanisms.

The engineered cells or delivery vehicles of the present disclosure may be used, in some instances, to treat cancer, such as ovarian cancer. Other cancers are described herein. For example, the engineered cells may be used to treat bladder tumors, brain tumors, breast tumors, cervical tumors, colorectal tumors, esophageal tumors, gliomas, kidney tumors, liver tumors, lung tumors, melanomas, ovarian tumors, pancreatic tumors, prostate tumors, skin tumors, thyroid tumors, and/or uterine tumors. The engineered cells or delivery vehicles of the present disclosure can be used to treat cancers with tumors located in the peritoneal space of a subject.

The methods provided herein also include delivering a preparation of engineered cells or delivery vehicles. A preparation, in some embodiments, is a substantially pure preparation, containing, for example, less than 5% (e.g., less than 4%, 3%, 2%, or 1%) of cells other than engineered cells. A preparation may comprise 1×10⁵ cells/kg to 1×10⁷ cells/kg cells. Preparation of engineered cells or delivery vehicles can include pharmaceutical compositions having one or more pharmaceutically acceptable carriers. For example, preparations of engineered cells or delivery vehicles can include any of the engineered viruses, such as an engineered AAV virus, or any of the engineered viral vectors, such as AAV vector, described herein.

### In Vivo Expression

The methods provided herein also include delivering a composition in vivo capable of producing the engineered cells described herein, e.g., capable of delivering any of the engineered nucleic acids described herein to a cell in vivo. Such compositions include any of the viral-mediated delivery platforms, any of the lipid structure delivery systems, any of the nanoparticle delivery systems, any of the genomic editing systems, or any of the other engineering delivery systems described herein capable of engineering a cell in vivo.

The methods provided herein also include delivering a composition in vivo capable of producing any of the proteins of interest described herein, e.g., any of the multicistronic systems encoding each of (i) a membrane-cleavable chimeric protein; (ii) a bivalent aCAR; and (iii) an iCAR described herein. The methods provided herein also include delivering a composition in vivo capable of producing two or more of the proteins of interest described herein. Compositions capable of in vivo production of proteins of interest include, but are not limited to, any of the engineered nucleic acids described herein. Compositions capable of in vivo production proteins of interest can be a naked mRNA or a naked plasmid.

### Enumerated Embodiments

Embodiment 1: A multicistronic expression system comprising an engineered nucleic acid comprising:
(A) an exogenous polynucleotide encoding a membrane-cleavable chimeric protein, oriented from N-terminal to C-terminal, having the formula:

S—C-MT or MT-C—S 1.
ii, wherein
iii. S comprises a secretable effector molecule, wherein the secretable effector molecule comprises IL-15,
iv. C comprises a protease cleavage site, and
v. MT comprises a cell membrane tethering domain,
vi, wherein S—C-MT or MT-C—S is configured to be expressed as a single polypeptide;
(B) an exogenous polynucleotide encoding an inhibitory chimeric antigen receptor (iCAR) comprising:
   (i) an antigen-binding domain specific for endomucin (EMCN);
   (ii) one or more intracellular inhibitory domains that inhibit an immune response; and
   (iii) one or more polypeptides selected from the group consisting of: a signal peptide, a transmembrane domain, a hinge domain, a spacer region, one or more peptide linkers, and combinations thereof; and
(C) an exogenous polynucleotide encoding a bivalent activating chimeric antigen receptor (aCAR) comprising:
   (i) an antigen-binding domain specific for FLT3;
   (ii) an antigen-binding domain specific for CD33;
   (iii) one or more intracellular signaling domains that stimulate an immune response; and
   (iv) one or more polypeptides selected from the group consisting of: a signal peptide, a transmembrane domain, a hinge domain, a spacer region, one or more peptide linkers, and combinations thereof.
      Embodiment 2: The multicistronic expression system of embodiment 1, wherein the exogenous polynucleotides encoding each of the membrane-cleavable chimeric protein, the iCAR, and the bivalent aCAR are linked together by a polynucleotide linker encoding a 2A ribosome skipping element.
      Embodiment 3: The multicistronic expression system of embodiment 2, wherein the 2A ribosome skipping element is selected from the group consisting of: a T2A ribosome skipping element, a E2A ribosome skipping element, a P2A ribosome skipping element, a F2A ribosome skipping element, ribosome skipping element fusions thereof, and combinations thereof.
      Embodiment 4: The multicistronic expression system of embodiment 3, wherein the ribosome skipping element fusion comprises an E2A/T2A ribosome skipping element.
      Embodiment 5: The multicistronic expression system of embodiment 4, wherein the E2A/T2A ribosome skipping element comprises the amino acid sequence QCTNYALLKLAGDVESNPGPGSGEGRGSLLTCGDVEENPGP (SEQ ID NO: 221).
      Embodiment 6: The multicistronic expression system of embodiment 5, wherein the E2A/T2A ribosome skipping element is encoded by a polynucleotide sequence comprising the sequence CAGTGTACCAACTACGCCCTGCTGAAACTGGCCGGCGACGTGGAATCTAATC CTGGACCTGGATCTGGCGAGGGACGCGGGAGTCTACTGACGTGTGGAGACG TGGAGGAAAACCCTGGACCT (SEQ ID NO: 222) or the sequence CAGTGCACAAATTATGCACTGCTGAAGCTCGCCGGGGATGTCGAGAGTAACC CAGGACCTGGAAGCGGAGAAGGTCGTGGTAGTCTACTAACGTGTGGTGATGT AGAAGAAAATCCTGGACCT (SEQ ID NO: 223).
      Embodiment 7: The multicistronic expression system of any one of embodiments 1-7, wherein the membrane-cleavable chimeric protein, the iCAR, and the bivalent aCAR are encoded in order from 5′ to 3′: (i) membrane-cleavable chimeric protein; (ii) bivalent aCAR; and (iii) iCAR.
      Embodiment 8: The multicistronic expression system of any one of embodiments 1-7, wherein the membrane-cleavable chimeric protein, the iCAR, and the bivalent aCAR are encoded in order from 5′ to 3′: (i) membrane-cleavable chimeric protein; (ii) iCAR; and (iii) bivalent aCAR.
      Embodiment 9: The multicistronic expression system of any one of embodiments 1-8, wherein the secretable effector molecule comprises a signal peptide or a signal-anchor sequence.
      Embodiment 10: The multicistronic expression system of embodiment 9, wherein the signal peptide comprises a native signal peptide native to the secretable effector molecule.
      Embodiment 11: The multicistronic expression system of embodiment 9, wherein the signal peptide comprises a non-native signal peptide or the signal-anchor sequence comprises a non-native signal-anchor sequence non-native to the secretable effector molecule.
      Embodiment 12: The multicistronic expression system of embodiment 11, wherein the non-native signal peptide or the non-native signal-anchor sequence is selected from the group consisting of: IgE, IL-12, IL-2, optimized IL-2, trypsiongen-2, Gaussia luciferase, CD5, human IgKVII, murine IgKVII, VSV-G, prolactin, serum albumin preprotein, azurocidin preprotein, osteonectin, CD33, IL-6, IL-8, CCL2, TIMP2, VEGFB, osteoprotegerin, serpin E1, GROalpha, CXCL12, IL-21, CD8, NKG2D, TNFR2, GMCSF, and GM-CSFRa.
      Embodiment 13: The multicistronic expression system of embodiment 11, wherein the non-native signal peptide comprises an IgE signal peptide.
      Embodiment 14: The multicistronic expression system of embodiment 13, wherein the IgE signal peptide comprises the amino acid sequence MDWTWILFLVAAATRVHS (SEQ ID NO: 228).
      Embodiment 15: The multicistronic expression system of embodiment 14, wherein the IgE signal peptide is encoded by a polynucleotide sequence comprising the sequence

(SEQ ID NO: 229)
ATGGACTGGACTTGGATACTCTTTCTGGTCGCTGCCGCCACACGGGTGCA
CTCT.

Embodiment 16: The multicistronic expression system of any one of embodiments 1-15, wherein the IL-15 comprises the amino acid sequence

(SEQ ID NO: 224)
NWVNVISDLKKIEDLIQSMHIDATLYTESDVHPSCKVTAMKCFLLELQVI
SLESGDASIHDTVENLIILANNSLSSNGNVTESGCKECEELEEKNIKEFL
QSFVHIVQMFINTS.

Embodiment 17: The multicistronic expression system of embodiment 16, wherein the IL-15 is encoded by a polynucleotide sequence comprising the sequence

(SEQ ID NO: 225)
AATTGGGTCAACGTGATCAGCGACCTGAAGAAGATCGAGGACCTGATCCA
GAGCATGCACATCGACGCCACACTGTACACCGAGTCCGATGTGCACCCTA
GCTGCAAAGTGACCGCCATGAAGTGCTTTCTGCTGGAACTGCAAGTGATC
AGCCTGGAAAGCGGCGACGCCAGCATCCACGATACCGTGGAAAATCTGAT
CATCCTGGCCAACAACAGCCTGTCCAGCAACGGCAATGTGACCGAGAGCG
GCTGCAAAGAGTGCGAGGAACTGGAAGAGAAGAACATCAAAGAGTTTCTG
CAGAGCTTCGTCCACATCGTGCAGATGTTCATCAACACCTCA.

Embodiment 18: The multicistronic expression system of any one of embodiments 1-17, wherein the protease cleavage site further comprises the N-terminal peptide linker, optionally selected from the group consisting of: SGGGGSGGGGSG (SEQ ID NO: 230); GGGSGGGGSGGGSLQ (SEQ ID NO: 231); GGS (SEQ ID NO: 232); GGSGGS (SEQ ID NO: 233); GGSGGSGGS (SEQ ID NO: 234); GGSGGSGGSGGS (SEQ ID NO: 235); GGSGGSGGSGGSGGS (SEQ ID NO: 236); GGGS (SEQ ID NO: 237); GGGSGGGS (SEQ ID NO: 238); GGGSGGGSGGGS (SEQ ID NO: 239); GGGSGGGSGGGSGGGS (SEQ ID NO: 240); GGGSGGGSGGGSGGGSGGGS (SEQ ID NO: 241); GGGGS (SEQ ID NO: 242); GGGGSGGGGS (SEQ ID NO: 243); GGGGSGGGGSGGGGS (SEQ ID NO: 244); GGGGSGGGGSGGGGSGGGGS (SEQ ID NO: 245); GGGGSGGGGSGGGGSGGGGSGGGGS (SEQ ID NO: 246); GSTSGSGKPGSGEGSTKG (SEQ ID NO: 247); and EAAAKEAAAKEAAAKEAAAK (SEQ ID NO: 248).
Embodiment 19: The multicistronic expression system of any one of embodiments 1-17, wherein the protease cleavage site further comprises the N-terminal peptide linker SGGGGSGGGGSG (SEQ ID NO: 230).
Embodiment 20: The multicistronic expression system of any one of embodiments 1-19, wherein the protease cleavage site further comprises a C-terminal peptide linker, optionally selected from the group consisting of: SGGGGSGGGGSG (SEQ ID NO: 230); GGGSGGGGSGGGSLQ (SEQ ID NO: 231); GGS (SEQ ID NO: 232); GGSGGS (SEQ ID NO: 233); GGSGGSGGS (SEQ ID NO: 234); GGSGGSGGSGGS (SEQ ID NO: 235); GGSGGSGGSGGSGGS (SEQ ID NO: 236); GGGS (SEQ ID NO: 237); GGGSGGGS (SEQ ID NO: 238); GGGSGGGSGGGS (SEQ ID NO: 239); GGGSGGGSGGGSGGGS (SEQ ID NO: 240); GGGSGGGSGGGSGGGSGGGS (SEQ ID NO: 241); GGGGS (SEQ ID NO: 242); GGGGSGGGGS (SEQ ID NO: 243); GGGGSGGGGSGGGGS (SEQ ID NO: 244); GGGGSGGGGSGGGGSGGGGS (SEQ ID NO: 245); GGGGSGGGGSGGGGSGGGGSGGGGS (SEQ ID NO: 246); GSTSGSGKPGSGEGSTKG (SEQ ID NO: 247); and EAAAKEAAAKEAAAKEAAAK (SEQ ID NO: 248).
Embodiment 21: The multicistronic expression system of any one of embodiments 1-19, wherein the protease cleavage site further comprises the C-terminal linker GGGSGGGGSGGGSLQ (SEQ ID NO: 231).
Embodiment 22: The multicistronic expression system of any one of embodiments 1-21, wherein the protease cleavage site comprises a Tumor Necrosis Factor-α Converting Enzyme (TACE)-specific cleavage site.
Embodiment 23: The multicistronic expression system of embodiment 22, wherein the TACE-specific cleavage site comprises the amino acid sequence VTPEPIFSLI (SEQ ID NO: 191).
Embodiment 24: The multicistronic expression system of embodiment 22, wherein the TACE-specific cleavage site comprises the amino acid sequence

(SEQ ID NO: 250)
SGGGGSGGGGSGVTPEPIFSLIGGGSGGGGSGGGSLQ.

Embodiment 25: The multicistronic expression system of embodiment 24, wherein the TACE-specific cleavage site is encoded by a polynucleotide sequence comprising the sequence

(SEQ ID NO: 251)
TCAGGCGGCGGTGGTAGTGGAGGCGGAGGCTCAGGCGTGACCCCTGAGCC
TATCTTCAGCCTGATCGGCGGAGGTTCCGGAGGTGGCGGTTCCGGCGGAG
GATCTCTTCAA.

Embodiment 26: The multicistronic expression system of any one of embodiments 1-25, wherein the cell membrane tethering domain comprises a transmembrane domain selected from the group consisting of: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, LIR1, B7-1, and BTLA.
Embodiment 27: The multicistronic expression system of any one of embodiments 1-25, wherein the cell membrane tethering domain comprises a B7-1 transmembrane domain.
Embodiment 28: The multicistronic expression system of embodiment 27, wherein the B7-1 transmembrane domain comprises the amino acid sequence

(SEQ ID NO: 204)
LLPSWAITLISVNGIFVICCLTYCFAPRCRERRRNERLRRESVRPV.

Embodiment 29: The multicistronic expression system of embodiment 28, wherein the B7-1 transmembrane domain is encoded by a polynucleotide sequence comprising the sequence

(SEQ ID NO: 252)
TTGCTGCCTAGCTGGGCCATCACACTGATCTCCGTGAACGGCATCTTCGT
GATCTGCTGCCTGACCTACTGCTTCGCCCCTAGATGCAGAGAGCGGAGAA
GAAACGAGCGGCTGAGAAGAGAAAGCGTGCGGCCTGTG.

Embodiment 30: The multicistronic expression system of any one of embodiments 1-29, wherein the membrane-cleavable chimeric protein, oriented from N-terminal to C-terminal, has the formula S—C-MT.
Embodiment 31: The multicistronic expression system of embodiment 30, wherein the membrane-cleavable chimeric protein comprises the amino acid sequence

(SEQ ID NO: 226)
MDWTWILFLVAAATRVHSNWVNVISDLKKIEDLIQSMHIDATLYTE
SDVHPSCKVTAMKCFLLELQVISLESGDASIHDTVENLIILANNSL
SSNGNVTESGCKECEELEEKNIKEFLQSFVHIVQMFINTSSGGGGS
GGGGSGVTPEPIFSLIGGGSGGGGSGGGSLQLLPSWAITLISVNGI
FVICCLTYCFAPRCRERRRNERLRRESVRPV.

Embodiment 32: The multicistronic expression system of embodiment 31, wherein the membrane-cleavable chimeric protein is encoded by a polynucleotide sequence comprising the sequence

(SEQ ID NO: 227)
ATGGACTGGACTTGGATACTCTTTCTGGTCGCTGCCGCCACACGGG
TGCACTCTAATTGGGTCAACGTGATCAGCGACCTGAAGAAGATCGA
GGACCTGATCCAGAGCATGCACATCGACGCCACACTGTACACCGAG
TCCGATGTGCACCCTAGCTGCAAAGTGACCGCCATGAAGTGCTTTC
TGCTGGAACTGCAAGTGATCAGCCTGGAAAGCGGCGACGCCAGCAT
CCACGATACCGTGGAAAATCTGATCATCCTGGCCAACAACAGCCTG
TCCAGCAACGGCAATGTGACCGAGAGCGGCTGCAAAGAGTGCGAGG
AACTGGAAGAGAAGAACATCAAAGAGTTTCTGCAGAGCTTCGTCCA
CATCGTGCAGATGTTCATCAACACCTCATCAGGCGGCGGTGGTAGT
GGAGGCGGAGGCTCAGGCGTGACCCCTGAGCCTATCTTCAGCCTGA
TCGGCGGAGGTTCCGGAGGTGGCGGTTCCGGCGGAGGATCTCTTCA
ATTGCTGCCTAGCTGGGCCATCACACTGATCTCCGTGAACGGCATC
TTCGTGATCTGCTGCCTGACCTACTGCTTCGCCCCTAGATGCAGAG
AGCGGAGAAGAAACGAGCGGCTGAGAAGAGAAAGCGTGCGGCCTGT
G.

Embodiment 33: The multicistronic expression system of any one of embodiments 1-32, wherein the antigen-binding domain specific for EMCN comprises a heavy chain variable (VH) region and a light chain variable (VL) region, wherein:
(A) the EMCN-VH comprises:
(B) a heavy chain complementarity determining region 1 (CDR-H1) having the amino acid sequence of RYDMH (SEQ ID NO: 291),
(C) a heavy chain complementarity determining region 2 (CDR-H2) having the amino acid sequence of VIWGNGNTHYHSALKS (SEQ ID NO: 296), and
(D) a heavy chain complementarity determining region 3 (CDR-H3) having the amino acid sequence of RIKD (SEQ ID NO: 298), and
(E) the EMCN-VL comprises:
(F) a light chain complementarity determining region 1 (CDR-L1) having the amino acid sequence of KSSQSLVASDENTYLN (SEQ ID NO: 299),
(G) a light chain complementarity determining region 2 (CDR-L2) having the amino acid sequence of QVSKLDS (SEQ ID NO: 300), and
(H) a light chain complementarity determining region 3 (CDR-L3) having the amino acid sequence of LQGIHLPWT (SEQ ID NO: 301), and
(I) wherein the amino acid sequences of the CDR-H1, the CDR-H2, the CDR-H3, the CDR-L1, the CDR-L2, and the CDR-L3 of the reference antibody are defined based on the Kabat numbering scheme.
   Embodiment 34: The multicistronic expression system of embodiment 33, wherein:

(SEQ ID NO: 302)

| | |
|---|---|
| (A) | the EMCN-VH comprises the amino acid sequence |
| | EVQLVESGGGLVQPGGSLRLSCAASGFTFSRYDMHWVRQAPGKGLE |
| | WVSVIWGNGNTHYHSALKSRFTISRDNSKNTLYLQMNSLRAEDTAV |
| | YYCTLRIKDWGQGTMVTVSS; |
| and | |

(SEQ ID NO: 310)

| | |
|---|---|
| (B) | the EMCN-VL comprises the amino acid sequence |
| | DVVMTQSPLSLPVTLGQPASISCKSSQSLVASDENTYLNWFQQRPG |
| | QSPRRLIYQVSKLDSGVPDRFSGSGSGTDFTLKISRVEAEDVGVYY |
| | CLQGIHLPWTFGQGTKLEIK. |

Embodiment 35: The multicistronic expression system of embodiment 33 or 34, wherein the EMCN-VH and the EMCN-VL are separated by a peptide linker.
Embodiment 36: The multicistronic expression system of embodiment 35, wherein the antigen-binding domain specific for EMCN comprises the structure VH-L-VL or VL-L-VH, wherein L is the peptide linker.
Embodiment 37: The multicistronic expression system of embodiment 36, wherein the peptide linker comprises the amino acid sequence SGGGGSGGGGSG (SEQ ID NO: 230); GGGSGGGGSGGGSLQ (SEQ ID NO: 231); GGS (SEQ ID NO: 232); GGSGGS (SEQ ID NO: 233); GGSGGSGGS (SEQ ID NO: 234); GGSGGSGGSGGS (SEQ ID NO: 235); GGSGGSGGSGGSGGS (SEQ ID NO: 236); GGGS (SEQ ID NO: 237); GGGSGGGS (SEQ ID NO: 238); GGGSGGGSGGGS (SEQ ID NO: 239); GGGSGGGSGGGSGGGS (SEQ ID NO: 240); GGGSGGGSGGGSGGGSGGGS (SEQ ID NO: 241); GGGGS (SEQ ID NO: 242); GGGGSGGGGS (SEQ ID NO: 243); GGGGSGGGGSGGGGS (SEQ ID NO: 244); GGGGSGGGGSGGGGSGGGGS (SEQ ID NO: 245); GGGGSGGGGSGGGGSGGGGSGGGGS (SEQ ID NO: 246); GSTSGSGKPGSGEGSTKG (SEQ ID NO: 247); or EAAAKEAAAKEAAAKEAAAK (SEQ ID NO: 248).
Embodiment 38: The multicistronic expression system of embodiment 36, wherein the peptide linker comprises the amino acid sequence GGGGSGGGGSGGGGS (SEQ ID NO: 244).
Embodiment 39: The multicistronic expression system of embodiment 38, wherein the peptide linker is encoded by a polynucleotide sequence comprising the sequence

(SEQ ID NO: 253)
GGAGGCGGAGGATCTGGTGGCGGAGGAAGTGGCGGAGGCGGTTCT.

Embodiment 40: The multicistronic expression system of any one of embodiments 1-39, wherein the antigen-binding domain specific for EMCN comprises the structure VH-L-VL and comprises the amino acid sequence

(SEQ ID NO: 311)
EVQLVESGGGLVQPGGSLRLSCAASGFTFSRYDMHWVRQAPGKGLE
WVSVIWGNGNTHYHSALKSRFTISRDNSKNTLYLQMNSLRAEDTAV
YYCTLRIKDWGQGTMVTVSSGGGGSGGGGSGGGGSDVVMTQSPLSL
PVTLGQPASISCKSSQSLVASDENTYLNWFQQRPGQSPRRLIYQVS
KLDSGVPDRFSGSGSGTDFTLKISRVEAEDVGVYYCLQGIHLPWTF
GQGTKLEIK.

Embodiment 41: The multicistronic expression system of embodiment 40, wherein the antigen-binding domain specific for EMCN is encoded by a polynucleotide sequence comprising the sequence

(SEQ ID NO: 312)
GAGGTGCAGCTGGTTGAATCTGGCGGAGGACTGGTTCAGCCTGGCG
GATCTCTGAGACTGTCTTGTGCCGCCAGCGGCTTCACCTTCAGCAG
ATACGATATGCACTGGGTCCGACAGGCCCCTGGCAAAGGACTTGAA
TGGGTGTCCGTGATCTGGGGCAACGGCAACACACACTACCACAGCG
CCCTGAAGTCCCGGTTCACCATCTCCAGAGACAACAGCAAGAACAC
CCTGTACCTGCAGATGAACAGCCTGAGAGCCGAGGACACCGCCGTG
TACTACTGCACCCTGAGAATCAAGGATTGGGGCCAGGGCACCATGG
TCACCGTTTCTTCTGGAGGCGGAGGATCTGGTGGCGGAGGAAGTGG
CGGAGGCGGTTCTGACGTGGTCATGACACAGAGCCCTCTGAGCCTG
CCTGTGACACTGGGACAGCCTGCCAGCATCAGCTGCAAGTCTAGCC
AGTCTCTGGTGGCCAGCGACGAGAACACCTACCTGAACTGGTTCCA
GCAGAGGCCCGGACAGTCTCCTAGACGGCTGATCTACCAGGTGTCC
AAGCTGGATAGCGGCGTGCCCGATAGATTTTCTGGCAGCGGCTCTG
GCACCGACTTCACCCTGAAGATCAGCAGAGTGGAAGCCGAGGACGT
GGGCGTGTACTACTGTCTGCAAGGCATCCATCTGCCTTGGACCTTT
GGCCAGGGCACAAAGCTGGAAATCAAG.

Embodiment 42: The multicistronic expression system of any one of embodiments 1-41, wherein the intracellular inhibitory domain comprises a LIR1 intracellular inhibitory domain.
Embodiment 43: The multicistronic expression system of embodiment 42, wherein the LIR1 intracellular inhibitory domain comprises the amino acid sequence

(SEQ ID NO: 285)
LRHRRQGKHWTSTQRKADFQHPAGAVGPEPTDRGLQWRSSPAADAQ
EENLYAAVKHTQPEDGVEMDTRSPHDEDPQAVTYAEVKHSRPRREM
ASPPSPLSGEFLDTKDRQAEEDRQMDTEAAASEAPQDVTYAQLHSL
TLRREATEPPPSQEGPSPAVPSIYATLAIH.

Embodiment 44: The multicistronic expression system of embodiment 43, wherein the LIR1 intracellular inhibitory domain is encoded by a polynucleotide sequence comprising the sequence

(SEQ ID NO: 286)
CTGCGGCACAGAAGGCAGGGCAAGCACTGGACAAGCACCCAGAGAA
AGGCCGACTTTCAGCATCCTGCTGGCGCCGTTGGACCTGAGCCTAC
AGATAGAGGACTGCAGTGGCGGTCTAGCCCTGCCGCTGATGCCCAA
GAGGAAAATCTTTACGCCGCCGTGAAGCACACCCAGCCTGAGGATG
GCGTGGAAATGGACACCAGATCTCCCCACGATGAGGACCCTCAGGC
CGTGACATACGCAGAAGTGAAGCACTCCAGACCTCGGAGAGAGATG
GCAAGCCCTCCATCTCCTCTGAGCGGCGAGTTCCTGGACACCAAAG
ACAGACAGGCCGAAGAGGACAGACAGATGGATACCGAAGCCGCCGC
TTCTGAAGCCCCACAGGATGTGACATATGCCCAGCTGCATAGCCTG
ACACTGCGGAGAGAAGCCACAGAGCCTCCACCTTCTCAAGAAGGCC
CATCTCCTGCCGTGCCTTCCATCTATGCCACTCTGGCCATTCAC.

Embodiment 45: The multicistronic expression system of any one of embodiments 1-44, wherein the signal peptide is present in the iCAR.
Embodiment 46: The multicistronic expression system of embodiment 45, wherein the signal peptide of the iCAR comprises a native signal peptide native or a non-native signal peptide, optionally wherein the non-native signal peptide or the non-native signal-anchor sequence is selected from the group consisting of: IgE, IL-12, IL-2, optimized IL-2, trypsiongen-2, Gaussia luciferase, CD5, human IgKVII, murine IgKVII, VSV-G, prolactin, serum albumin preprotein, azurocidin preprotein, osteonectin, CD33, IL-6, IL-8, CCL2, TIMP2, VEGFB, osteoprotegerin, serpin E1, GROalpha, CXCL12, IL-21, CD8, NKG2D, TNFR2, and GMCSF.
Embodiment 47: The multicistronic expression system of embodiment 45, wherein the signal peptide of the iCAR comprises a CD8 signal peptide.
Embodiment 48: The multicistronic expression system of embodiment 47, wherein the CD8 signal peptide comprises the amino acid sequence

(SEQ ID NO: 137)
MALPVTALLLPLALLLHAARP.

Embodiment 49: The multicistronic expression system of embodiment 48, wherein the CD8 signal peptide is encoded by a polynucleotide sequence comprising the sequence

(SEQ ID NO: 416)
ATGGCTCTGCCCGTGACAGCTTTGCTGCTGCCTTTGGCACTGCTGC
TGCATGCTGCTAGACCA.

Embodiment 50: The multicistronic expression system of any one of embodiments 1-49, wherein the hinge domain is present in the iCAR comprises.
Embodiment 51: The multicistronic expression system of embodiment 50, wherein the hinge domain of the iCAR is selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof.
Embodiment 52: The multicistronic expression system of embodiment 50, wherein the hinge domain of the iCAR comprises a LIR1 hinge.
Embodiment 53: The multicistronic expression system of embodiment 52, wherein the LIR1 hinge comprises the amino acid sequence

(SEQ ID NO: 417)
HPSDPLELVVSGPSGGPSSPTTGPTSTSGPEDQPLTPTGSDPQSGL
GRHLGV.

Embodiment 54: The multicistronic expression system of embodiment 53, wherein the LIR1 hinge comprises is encoded by a polynucleotide sequence comprising the sequence

(SEQ ID NO: 418)
CACCCATCCGATCCTCTCGAGCTGGTGGTTTCTGGACCTTCTGGCG
GCCCTAGCAGCCCTACAACAGGACCTACAAGCACAAGCGGCCCTGA
GGACCAACCTCTGACACCAACAGGCAGCGATCCTCAGTCTGGACTG
GGGAGACATCTGGGCGTT.

Embodiment 55: The multicistronic expression system of embodiment 50, wherein the hinge domain of the iCAR comprises a CD8 hinge.
Embodiment 56: The multicistronic expression system of embodiment 55, wherein the CD8 hinge comprises the amino acid sequence

(SEQ ID NO: 271)
TTTPAPRPPTPAPTIALQPLSLRPEACRPAAGGAVHTRGLDFACD.

Embodiment 57: The multicistronic expression system of embodiment 56, wherein the CD8 hinge comprises is encoded by a polynucleotide sequence comprising the sequence

(SEQ ID NO: 283)
ACAACAACACCCGCACCTCGGCCTCCAACTCCAGCTCCAACAATTG
CACTGCAACCCCTGAGTCTGAGGCCCGAGGCCTGTAGGCCAGCAGC
TGGCGGAGCTGTTCACACTAGAGGCCTGGACTTTGCCTGTGAC.

Embodiment 58: The multicistronic expression system of any one of embodiments 1-57, wherein the transmembrane domain of the iCAR is present.
Embodiment 59: The multicistronic expression system of embodiment 58, wherein the transmembrane domain of the iCAR comprises a transmembrane domain selected from the group consisting of: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, LIR1, and BTLA.
Embodiment 60: The multicistronic expression system of embodiment 58, wherein the transmembrane domain of the iCAR comprises a LIR 1 transmembrane domain.
Embodiment 61: The multicistronic expression system of embodiment 60, wherein the LIR1 transmembrane domain comprises the amino acid sequence

(SEQ ID NO: 259)
VIGILVAVILLLLLLLLLFLI.

Embodiment 62: The multicistronic expression system of embodiment 61, wherein the LIR1 transmembrane domain is encoded by a polynucleotide sequence comprising the sequence

(SEQ ID NO: 260)
GTGATCGGCATTCTGGTCGCCGTGATCCTGCTCCTGTTGCTCCTGCTGCT
TCTGTTCCTGATC.

Embodiment 63: The multicistronic expression system of any one of embodiments 1-62, wherein the iCAR comprises the amino acid sequence

(A)
(SEQ ID NO: 430)
MALPVTALLLPLALLLHAARPEVQLVESGGGLVQPGGSLRLSCAASG
FTFSRYDMHWVRQAPGKGLEWVSVIWGNGNTHYHSALKSRFTISRD
NSKNTLYLQMNSLRAEDTAVYYCTLRIKDWGQGTMVTVSSGGGGSG
GGGSGGGGSDVVMTQSPLSLPVTLGQPASISCKSSQSLVASDENTYLN
WFQQRPGQSPRRLIYQVSKLDSGVPDRFSGSGSGTDFTLKISRVEAED
VGVYYCLQGIHLPWTFGQGTKLEIKHPSDPLELVVSGPSGGPSSPTTG
PTSTSGPEDQPLTPTGSDPQSGLGRHLGVVIGILVA VILLLLLLLLLFL
ILRHRRQGKHWTSTQRKADFQHPAGAVGPEPTDRGLQWRSSPAADA
QEENLYAAVKHTQPEDGVEMDTRSPHDEDPQAVTYAEVKHSRPRRE
MASPPSPLSGEFLDTKDRQAEEDRQMDTEAAASEAPQDVTYAQLHSL
TLRREATEPPPSQEGPSPAVPSIYATLAIH;
or
(B)
(SEQ ID NO: 419)
MALPVTALLLPLALLLHAARPEVQLVESGGGLVQPGGSLRLSCAASG
FTFSRYDMHWVRQAPGKGLEWVSVIWGNGNTHYHSALKSRFTISRD
NSKNTLYLQMNSLRAEDTAVYYCTLRIKDWGQGTMVTVSSGGGGSG
GGGSGGGGSDVVMTQSPLSLPVTLGQPASISCKSSQSLVASDENTYLN
WFQQRPGQSPRRLIYQVSKLDSGVPDRFSGSGSGTDFTLKISRVEAED
VGVYYCLQGIHLPWTFGQGTKLEIKngaaHPSDPLELVVSGPSGGPSSP
TTGPTSTSGPEDQPLTPTGSDPQSGLGRHLGVVIGILVAVILLLLLLLLL
FLILRHRRQGKHWTSTQRKADFQHPAGAVGPEPTDRGLQWRSSPAA
DAQEENLYAAVKHTQPEDGVEMDTRSPHDEDPQAVTYAEVKHSRPR
REMASPPSPLSGEFLDTKDRQAEEDRQMDTEAAASEAPQDVTYAQLH
SLTLRREATEPPPSQEGPSPAVPSIYATLAIH.

Embodiment 64: The multicistronic expression system of embodiment 63, wherein the iCAR is encoded by a polynucleotide sequence comprising the sequence

(SEQ ID NO: 420)
ATGGCTCTGCCCGTGACAGCTTTGCTGCTGCCTTTGGCACTGCTGCTGCA
TGCTGCTAGACCAGAGGTGCAGCTGGTTGAATCTGGCGGAGGACTGGTTC
AGCCTGGCGGATCTCTGAGACTGTCTTGTGCCGCCAGCGGCTTCACCTTC
AGCAGATACGATATGCACTGGGTCCGACAGGCCCCTGGCAAAGGACTTGA
ATGGGTGTCCGTGATCTGGGGCAACGGCAACACACACTACCACAGCGCCC
TGAAGTCCCGGTTCACCATCTCCAGAGACAACAGCAAGAACACCCTGTAC
CTGCAGATGAACAGCCTGAGAGCCGAGGACACCGCCGTGTACTACTGCAC
CCTGAGAATCAAGGATTGGGGCCAGGGCACCATGGTCACCGTTTCTTCTG
GAGGCGGAGGATCTGGTGGCGGAGGAAGTGGCGGAGGCGGTTCTGACGTG
GTCATGACACAGAGCCCTCTGAGCCTGCCTGTGACACTGGGACAGCCTGC
CAGCATCAGCTGCAAGTCTAGCCAGTCTCTGGTGGCCAGCGACGAGAACA
CCTACCTGAACTGGTTCCAGCAGAGGCCCGGACAGTCTCCTAGACGGCTG
ATCTACCAGGTGTCCAAGCTGGATAGCGGCGTGCCCGATAGATTTTCTGG
CAGCGGCTCTGGCACCGACTTCACCCTGAAGATCAGCAGAGTGGAAGCCG
AGGACGTGGGCGTGTACTACTGTCTGCAAGGCATCCATCTGCCTTGGACC
TTTGGCCAGGGCACAAAGCTGGAAATCAAGAATGGCGCTGCACACCCATC
CGATCCTCTCGAGCTGGTGGTTTCTGGACCTTCTGGCGGCCCTAGCAGCC
CTACAACAGGACCTACAAGCACAAGCGGCCCTGAGGACCAACCTCTGACA
CCAACAGGCAGCGATCCTCAGTCTGGACTGGGGAGACATCTGGGCGTTGT
GATCGGCATTCTGGTCGCCGTGATCCTGCTCCTGTTGCTCCTGCTGCTTC
TGTTCCTGATCCTGCGGCACAGAAGGCAGGGCAAGCACTGGACAAGCACC
CAGAGAAAGGCCGACTTTCAGCATCCTGCTGGCGCCGTTGGACCTGAGCC
TACAGATAGAGGACTGCAGTGGCGGTCTAGCCCTGCCGCTGATGCCCAAG
AGGAAAATCTTTACGCCGCCGTGAAGCACACCCAGCCTGAGGATGGCGTG
GAAATGGACACCAGATCTCCCCACGATGAGGACCCTCAGGCCGTGACATA
CGCAGAAGTGAAGCACTCCAGACCTCGGAGAGAGATGGCAAGCCCTCCAT
CTCCTCTGAGCGGCGAGTTCCTGGACACCAAAGACAGACAGGCCGAAGAG
GACAGACAGATGGATACCGAAGCCGCCGCTTCTGAAGCCCCACAGGATGT
GACATATGCCCAGCTGCATAGCCTGACACTGCGGAGAGAAGCCACAGAGC
CTCCACCTTCTCAAGAAGGCCCATCTCCTGCCGTGCCTTCCATCTATGCC
ACTCTGGCCATTCAC.

Embodiment 65: The multicistronic expression system of any one of embodiments 1-62, wherein the iCAR comprises the amino acid sequence

(A)
(SEQ ID NO: 431)
MALPVTALLLPLALLLHAARPEVOLVESGGGLVQPGGSLRLSCAASG
FTFSRYDMHWVRQAPGKGLEWVSVIWGNGNTHYHSALKSRFTISRD
NSKNTLYLQMNSLRAEDTAVYYCTLRIKDWGQGTMVTVSSGGGGSG
GGGSGGGGSDVVMTQSPLSLPVTLGQPASISCKSSQSLVASDENTYLN
WFQQRPGQSPRRLIYQVSKLDSGVPDRFSGSGSGTDFTLKISRVEAED
VGVYYCLQGIHLPWTFGQGTKLEIKTTTPAPRPPTPAPTIALQPLSLRP
EACRPAAGGAVHTRGLDFACDVIGILVAVILLLLLLLLLFLILRHRRQG
KHWTSTQRKADFQHPAGAVGPEPTDRGLQWRSSPAADAQEENLYAA
VKHTQPEDGVEMDTRSPHDEDPQAVTYAEVKHSRPRREMASPPSPLS
GEFLDTKDRQAEEDRQMDTEAAASEAPQDVTYAQLHSLTLRREATEP
PPSQEGPSPAVPSIYATLAIH;
or
(B)
(SEQ ID NO: 421)
MALPVTALLLPLALLLHAARPEVQLVESGGGLVQPGGSLRLSCAASG
FTFSRYDMHWVRQAPGKGLEWVSVIWGNGNTHYHSALKSRFTISRD
NSKNTLYLQMNSLRAEDTAVYYCTLRIKDWGQGTMVTVSSGGGGSG
GGGSGGGGSDVVMTQSPLSLPVTLGQPASISCKSSQSLVASDENTYLN
WFQQRPGQSPRRLIYQVSKLDSGVPDRFSGSGSGTDFTLKISRVEAED
VGVYYCLQGIHLPWTFGQGTKLEIKngaaTTTPAPRPPTPAPTIALQPLS
LRPEACRPAAGGAVHTRGLDFACDVIGILVAVILLLLLLLLLFLILRHR
RQGKHWTSTQRKADFQHPAGAVGPEPTDRGLQWRSSPAADAQEENL
YAAVKHTQPEDGVEMDTRSPHDEDPQAVTYAEVKHSRPRREMASPP
SPLSGEFLDTKDRQAEEDRQMDTEAAASEAPQDVTYAQLHSLTLRRE
ATEPPPSQEGPSPAVPSIYATLAIH.

Embodiment 66: The multicistronic expression system of embodiment 65, wherein the iCAR is encoded by a polynucleotide sequence comprising the sequence

(SEQ ID NO: 422)
ATGGCTCTGCCCGTGACAGCTTTGCTGCTGCCTTTGGCACTGCTGCTGCA
TGCTGCTAGACCAGAGGTGCAGCTGGTTGAATCTGGCGGAGGACTGGTTC
AGCCTGGCGGATCTCTGAGACTGTCTTGTGCCGCCAGCGGCTTCACCTTC
AGCAGATACGATATGCACTGGGTCCGACAGGCCCCTGGCAAAGGACTTGA
ATGGGTGTCCGTGATCTGGGGCAACGGCAACACACACTACCACAGCGCCC
TGAAGTCCCGGTTCACCATCTCCAGAGACAACAGCAAGAACACCCTGTAC
CTGCAGATGAACAGCCTGAGAGCCGAGGACACCGCCGTGTACTACTGCAC
CCTGAGAATCAAGGATTGGGGCCAGGGCACCATGGTCACCGTTTCTTCTG
GAGGCGGAGGATCTGGTGGCGGAGGAAGTGGCGGAGGCGGTTCTGACGTG
GTCATGACACAGAGCCCTCTGAGCCTGCCTGTGACACTGGGACAGCCTGC
CAGCATCAGCTGCAAGTCTAGCCAGTCTCTGGTGGCCAGCGACGAGAACA
CCTACCTGAACTGGTTCCAGCAGAGGCCCGGACAGTCTCCTAGACGGCTG
ATCTACCAGGTGTCCAAGCTGGATAGCGGCGTGCCCGATAGATTTTCTGG
CAGCGGCTCTGGCACCGACTTCACCCTGAAGATCAGCAGAGTGGAAGCCG
AGGACGTGGGCGTGTACTACTGTCTGCAAGGCATCCATCTGCCTTGGACC
TTTGGCCAGGGCACAAAGCTGGAAATCAAGAATGGCGCTGCAACAACAAC
ACCCGCACCTCGGCCTCCAACTCCAGCTCCAACAATTGCACTGCAACCCC
TGAGTCTGAGGCCCGAGGCCTGTAGGCCAGCAGCTGGCGGAGCTGTTCAC
ACTAGAGGCCTGGACTTTGCCTGTGACGTGATCGGCATTCTGGTCGCCGT
GATCCTGCTCCTGTTGCTCCTGCTGCTTCTGTTCCTGATCCTGCGGCACA
GAAGGCAGGGCAAGCACTGGACAAGCACCCAGAGAAAGGCCGACTTTCAG
CATCCTGCTGGCGCCGTTGGACCTGAGCCTACAGATAGAGGACTGCAGTG
GCGGTCTAGCCCTGCCGCTGATGCCCAAGAGGAAAATCTTTACGCCGCCG
TGAAGCACACCCAGCCTGAGGATGGCGTGGAAATGGACACCAGATCTCCC
CACGATGAGGACCCTCAGGCCGTGACATACGCAGAAGTGAAGCACTCCAG
ACCTCGGAGAGAGATGGCAAGCCCTCCATCTCCTCTGAGCGGCGAGTTCC
TGGACACCAAAGACAGACAGGCCGAAGAGGACAGACAGATGGATACCGAA
GCCGCCGCTTCTGAAGCCCCACAGGATGTGACATATGCCCAGCTGCATAG
CCTGACACTGCGGAGAGAAGCCACAGAGCCTCCACCTTCTCAAGAAGGCC
CATCTCCTGCCGTGCCTTCCATCTATGCCACTCTGGCCATTCAC.

Embodiment 67: The multicistronic expression system of any one of embodiments 1-66, wherein the antigen-binding domain specific for FLT3 comprises a heavy chain variable (VH) region and a light chain variable (VL) region, wherein:
(A) the FLT3-VH comprises:
(B) a heavy chain complementarity determining region 1 (CDR-H1) having the amino acid sequence of GGTFSSYAIS (SEQ ID NO: 360),
(C) a heavy chain complementarity determining region 2 (CDR-H2) having the amino acid sequence of GIIPIFGTANYAQKFQG (SEQ ID NO: 361), and
(D) a heavy chain complementarity determining region 3 (CDR-H3) having the amino acid sequence of FALFGFREQAFDI (SEQ ID NO: 362), and the FLT3-VL comprises: (E)
(F) a light chain complementarity determining region 1 (CDR-L1) having the amino acid sequence of RASQSISSYLN (SEQ ID NO: 363),
(G) a light chain complementarity determining region 2 (CDR-L2) having the amino acid sequence of AASSLQS (SEQ ID NO: 364), and
(H) a light chain complementarity determining region 3 (CDR-L3) having the amino acid sequence of QQSYSTPFT (SEQ ID NO: 365), and
(I) wherein the amino acid sequences of the CDR-H1, the CDR-H2, the CDR-H3, the CDR-L1, the CDR-L2, and the CDR-L3 of the reference antibody are defined based on the Kabat numbering scheme.
   Embodiment 68: The multicistronic expression system of embodiment 67, wherein:

(A) the FLT3-VH comprises the amino acid sequence
(SEQ ID NO: 315)
EVQLVQSGAEVKKPGSSVKVSCKASGGTFSSYAISWVRQAPGQGLE
WMGGIIPIFGTANYAQKFQGRVTITADKSTSTAYMELSSLRSEDTAVY
YCATFALFGFREQAFDIWGQGTTVTVSS;
and
(B) the FLT3-VL comprises the amino acid sequence
(SEQ ID NO: 316)
DIQMTQSPSSLSASVGDRVTITCRASQSISSYLNWYQQKPGKAPKLLIY
AASSLQSGVPSRFSGSGSGTDFTLTISSLQPEDLATYYCQQSYSTPFTF
GPGTKVDIK.

Embodiment 69: The multicistronic expression system of any one of embodiments 1-68, wherein the antigen-binding domain specific for CD33 comprises a heavy chain variable (VH) region and a light chain variable (VL) region, wherein:
(A) the CD33-VH comprises:
(B) a heavy chain complementarity determining region 1 (CDR-H1) having the amino acid sequence of DYNMH (SEQ ID NO: 402),
(C) a heavy chain complementarity determining region 2 (CDR-H2) having the amino acid sequence of YIYPYNGGTGYNQKFKSKA (SEQ ID NO: 403), and
(D) a heavy chain complementarity determining region 3 (CDR-H3) having the amino acid sequence of GRPAMDYWGQ (SEQ ID NO: 404), and
(E) the CD33-VL comprises:
(F) a light chain complementarity determining region 1 (CDR-L1) having the amino acid sequence of RASESVDNYGISFMN (SEQ ID NO: 405),
(G) a light chain complementarity determining region 2 (CDR-L2) having the amino acid sequence of AASNQGS (SEQ ID NO: 406), and
(H) a light chain complementarity determining region 3 (CDR-L3) having the amino acid sequence of QQSKEVPWT (SEQ ID NO: 407), and
(I) wherein the amino acid sequences of the CDR-H1, the CDR-H2, the CDR-H3, the CDR-L1, the CDR-L2, and the CDR-L3 of the reference antibody are defined based on the Kabat numbering scheme.
   Embodiment 70: The multicistronic expression system of embodiment 69, wherein:

(A) the CD33-VH comprises the amino acid sequence
(SEQ ID NO: 329)
QVQLVQSGAEVKKPGSSVKVSCKASGYTFTDYNMHWVRQAPGQGL
EWIGYIYPYNGGTGYNQKFKSKATITADESTNTAYMELSSLRSEDTA
VYYCARGRPAMDYWGQGTLVTVSS;
and
(B) the CD33-VL comprises the amino acid sequence
(SEQ ID NO: 330)
DIQMTQSPSSLSASVGDRVTITCRASESVDNYGISFMNWFQQKPGKAP
KLLIYAASNQGSGVPSRFSGSGSGTDFTLTISSLQPDDFATYYCQQSKE
VPWTFGQGTKVEIK.

Embodiment 71: The multicistronic expression system of any one of embodiments 67-70, wherein the FLT3-VH, the FLT3-VL, the CD33-VH, and the CD33-VL are separated by peptide linkers.
Embodiment 72: The multicistronic expression system of embodiment 71, wherein the aCAR antigen binding domains comprises the structure (FLT3-VH)-L1-(CD33-VH)-L2-(CD33-VL)-L3-(FLT3-VL), wherein L1, L2, and L3 are a first, a second, and a third peptide linker, respectively.
Embodiment 73: The multicistronic expression system of embodiment 72, wherein the L1, L2, and/or L3 are each independently selected from the group consisting of: SEQ ID Nos 230-248.
Embodiment 74: The multicistronic expression system of embodiment 72, wherein the L1 peptide linker is the amino acid sequence GGGGS (SEQ ID NO: 242) or GGGGSGGGGS (SEQ ID NO: 243).
Embodiment 75: The multicistronic expression system of embodiment 73, wherein the L1 peptide linker GGGGS (SEQ ID NO: 242) is encoded by a polynucleotide sequence comprising the sequence GGCGGCGGTGGCTCT (SEQ ID NO: 254) or the L1 peptide linker GGGGSGGGGS (SEQ ID NO: 243) is encoded by a polynucleotide sequence comprising the sequence GGAGGCGGAGGATCTGGTGGTGGTGGATCT (SEQ ID NO: 256).
Embodiment 76: The multicistronic expression system of any one of embodiments 72-75, wherein the L2 peptide linker is the amino acid sequence GSTSGSGKPGSGEGSTKG (SEQ ID NO: 247).
Embodiment 77: The multicistronic expression system of embodiment 76, wherein the L2 peptide linker is encoded by a polynucleotide sequence comprising the sequence

(SEQ ID NO: 249)
GGCTCTACATCTGGCTCTGGCAAACCTGGAAGCGGCGAGGGATCTACCAA
GGGC.

Embodiment 78: The multicistronic expression system of any one of embodiments 72-75, wherein the L2 peptide linker is the amino acid sequence GGGGSGGGGS (SEQ ID NO: 243).
Embodiment 79: The multicistronic expression system of embodiment 76, wherein the L2 peptide linker is encoded by a polynucleotide sequence comprising the sequence

(SEQ ID NO: 257)
GGTGGCGGAGGAAGTGGCGGCGGAGGCTCT.

Embodiment 80: The multicistronic expression system of any one of embodiments 72-79, wherein the L3 peptide linker is the amino acid sequence GGGGS (SEQ ID NO: 242) or GGGGSGGGGS (SEQ ID NO: 243).
Embodiment 81: The multicistronic expression system of embodiment 80, wherein the L3 peptide linker GGGGS (SEQ ID NO: 242) is encoded by a polynucleotide sequence comprising the sequence GGTGGCGGCGGATCC (SEQ ID NO: 255) or the L3 peptide linker GGGGSGGGGS (SEQ ID NO: 243) is encoded by a polynucleotide sequence comprising the sequence GGCGGTGGCGGATCTGGCGGAGGTGGCAGT (SEQ ID NO: 258).
Embodiment 82: The multicistronic expression system of any one of embodiments 1-81, wherein the aCAR intracellular signaling domains that stimulate an immune response is selected from the group consisting of: CD3-zeta, FcR gamma, FcR beta, CD3 gamma, CD3 delta, CD3 epsilon, CD5, CD22, CD79a, CD79b, CD278, FcεRI, DAP10, DAP12, CD66d, CD97, CD2, ICOS, CD27, CD154, CD8, OX40, 4-1BB, CD28, ZAP40, CD30, GITR, HVEM, DAP10, DAP12, MyD88, 2B4, CD40, PD-1, LFA-1, CD7, LIGHT, NKG2C, B7-H3, an MHC class I molecule, a TNF receptor protein, an Immunoglobulin-like protein, a cytokine receptor, an integrin, a SLAM protein, an activating NK cell receptor, BTLA, a Toll ligand receptor, CDS, ICAM-1, (CD11a/CD18), BAFFR, KIRDS2, SLAMF7, NKp80 (KLRF1), NKp44, NKp30, NKp46, CD19, CD4, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and combinations thereof.
Embodiment 83: The multicistronic expression system of any one of embodiments 1-81, wherein the aCAR intracellular signaling domains that stimulate an immune response comprise a CD28 co-stimulatory domain and a CD3ζ signaling domain.
Embodiment 84: The multicistronic expression system of embodiment 83, wherein the CD28 co-stimulatory domain comprises the amino acid sequence

(SEQ ID NO: 287)
RSKRSRLLHSDYMNMTPRRPGPTRKHYQPYAPPRDFAAYRS.

Embodiment 85: The multicistronic expression system of embodiment 84, wherein the CD28 co-stimulatory domain is encoded by a polynucleotide sequence comprising the sequence

(SEQ ID NO: 288)
AGAAGCAAGCGGAGCAGACTGCTGCACAGCGACTACATGAACATGACCCC
TAGACGGCCCGGACCTACCAGAAAGCACTACCAGCCTTACGCTCCTCCTA
GAGATTTCGCCGCCTACCGGTCC.

Embodiment 86: The multicistronic expression system of embodiment 83, wherein the CD3ζ signaling domain comprises the amino acid sequence

(SEQ ID NO: 289)
RVKFSRSADAPAYKQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

Embodiment 87: The multicistronic expression system of embodiment 86, wherein the CD3ζ signaling domain is encoded by a polynucleotide sequence comprising the sequence

(SEQ ID NO: 290)
AGAGTGAAGTTCAGCAGGAGCGCAGACGCCCCCGCGTACAAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGC.

Embodiment 88: The multicistronic expression system of any one of embodiments 1-87, wherein the hinge domain of the aCAR is present.
Embodiment 89: The multicistronic expression system of embodiment 88, wherein the hinge domain of the aCAR is selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof.
Embodiment 90: The multicistronic expression system of embodiment 88, wherein the hinge domain of the aCAR comprises a CD8 hinge.
Embodiment 91: The multicistronic expression system of embodiment 90, wherein the CD8 hinge comprises the amino acid sequence

(SEQ ID NO: 272)
ALSNSIMYFSHFVPVFLPAKPTTTPAPRPPTPAPTIASQPLSLRPEACRP
AAGGAVHTRGLDFACD.

Embodiment 92: The multicistronic expression system of embodiment 91, wherein the CD8 hinge comprises is encoded by a polynucleotide sequence comprising the sequence

(SEQ ID NO: 284)
GCCCTGAGCAACAGCATCATGTACTTCAGCCACTTCGTGCCCGTGTTTCT
GCCCGCCAAGCCTACAACAACCCCTGCTCCTAGACCACCTACACCAGCTC
CTACAATCGCCAGCCAGCCTCTGTCTCTGAGGCCCGAAGCTTGTAGACCA
GCTGCTGGCGGAGCCGTGCATACAAGAGGACTGGATTTTGCCTGCGAC.

Embodiment 93: The multicistronic expression system of any one of embodiments 1-92, wherein the transmembrane domain of the aCAR is present.
Embodiment 94: The multicistronic expression system of embodiment 93, wherein the transmembrane domain of the aCAR is selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof.
Embodiment 95: The multicistronic expression system of embodiment 93, wherein the transmembrane domain of the aCAR comprises a CD8 hinge.
Embodiment 96: The multicistronic expression system of embodiment 95, wherein the CD8 transmembrane comprises the amino acid sequence

(SEQ ID NO: 206)
IYIWAPLAGTCGVLLLSLVITLYCNHR.

Embodiment 97: The multicistronic expression system of embodiment 96, wherein the CD8 transmembrane comprises is encoded by a polynucleotide sequence comprising the sequence

(SEQ ID NO: 261)
ATCTATATCTGGGCCCCTCTGGCTGGCACATGCGGAGTTCTGCTGCTCAG
CCTGGTCATCACCCTGTACTGCAACCACAGA.

Embodiment 98: The multicistronic expression system of any one of embodiments 1-97, wherein the aCAR signal peptide of the aCAR is present.
Embodiment 99: The multicistronic expression system of embodiment 98, wherein the signal peptide of the aCAR is selected from the group consisting of: IgE, IL-12, IL-2, optimized IL-2, trypsiongen-2, Gaussia luciferase, CD5, human IgKVII, murine IgKVII, VSV-G, prolactin, serum albumin preprotein, azurocidin preprotein, osteonectin, CD33, IL-6, IL-8, CCL2, TIMP2, VEGFB, osteoprotegerin, serpin E1, GROalpha, CXCL12, IL-21, CD8, NKG2D, TNFR2, GMCSF, and GM-CSFRa.
Embodiment 100: The multicistronic expression system of embodiment 98, wherein the signal peptide of the aCAR comprises a GM-CSFRa signal peptide.
Embodiment 101: The multicistronic expression system of embodiment 100, wherein the GM-CSFRa signal peptide comprises the amino acid sequence MLLLVTSLLLCELPHPAFLLIP (SEQ ID NO: 423).
Embodiment 102: The multicistronic expression system of embodiment 101, wherein the GM-CSFRa signal peptide is encoded by a polynucleotide sequence comprising the sequence

(SEQ ID NO: 424)
ATGCTGCTGCTGGTTACATCTCTGCTGCTGTGCGAGCTGCCCCATCCTGC
CTTTCTGCTTATTCCT.

Embodiment 103: The multicistronic expression system of any one of embodiments 1-103, wherein the aCAR comprises the amino acid sequence

(SEQ ID NO: 414)
MLLLVTSLLLCELPHPAFLLIPEVQLVQSGAEVKKPGSSVKVSCKASGGT
FSSYAISWVRQAPGQGLEWMGGIIPIFGTANYAQKFQGRVTITADKSTST
AYMELSSLRSEDTAVYYCATFALFGFREQAFDIWGQGTTVTVSSGGGGSQ
VQLVQSGAEVKKPGSSVKVSCKASGYTFTDYNMHWVRQAPGQGLEWIGYI
YPYNGGTGYNQKFKSKATITADESTNTAYMELSSLRSEDTAVYYCARGRP
AMDYWGQGTLVTVSSGSTSGSGKPGSGEGSTKGDIQMTQSPSSLSASVGD
RVTITCRASESVDNYGISFMNWFQQKPGKAPKLLIYAASNQGSGVPSRFS
GSGSGTDFTLTISSLQPDDFATYYCQQSKEVPWTFGQGTKVEIKGGGGSD
IQMTQSPSSLSASVGDRVTITCRASQSISSYLNWYQQKPGKAPKLLIYAA
SSLQSGVPSRFSGSGSGTDFTLTISSLQPEDLATYYCQQSYSTPFTFGPG
TKVDIKALSNSIMYFSHFVPVFLPAKPTTTPAPRPPTPAPTIASQPLSLR
PEACRPAAGGAVHTRGLDFACDIYIWAPLAGTCGVLLLSLVITLYCNHRR
SKRSRLLHSDYMNMTPRRPGPTRKHYQPYAPPRDFAAYRSRVKFSRSADA
PAYKQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR.

Embodiment 104: The multicistronic expression system of any one of embodiments 1-103, wherein the aCAR comprises the amino acid sequence

(SEQ ID NO: 415)
MLLLVTSLLLCELPHPAFLLIPEVQLVQSGAEVKKPGSSVKVSCKASGGT
ESSYAISWVRQAPGQGLEWMGGIIPIFGTANYAQKFQGRVTITADKSTST
AYMELSSLRSEDTAVYYCATFALFGFREQAFDIWGQGTTVTVSSGGGGSG
GGGSQVQLVQSGAEVKKPGSSVKVSCKASGYTFTDYNMHWVRQAPGQGLE
WIGYIYPYNGGTGYNQKFKSKATITADESTNTAYMELSSLRSEDTAVYYC
ARGRPAMDYWGQGTLVTVSSGGGGSGGGGSDIQMTQSPSSLSASVGDRVT
ITCRASESVDNYGISFMNWFQQKPGKAPKLLIYAASNQGSGVPSRFSGSG
SGTDFTLTISSLQPDDFATYYCQQSKEVPWTFGQGTKVEIKGGGGSGGGG
SDIQMTQSPSSLSASVGDRVTITCRASQSISSYLNWYQQKPGKAPKLLIY
AASSLQSGVPSRFSGSGSGTDFTLTISSLQPEDLATYYCQQSYSTPFTFG
PGTKVDIKALSNSIMYFSHFVPVFLPAKPTTTPAPRPPTPAPTIASQPLS
LRPEACRPAAGGAVHTRGLDFACDIYIWAPLAGTCGVLLLSLVITLYCNH
RRSKRSRLLHSDYMNMTPRRPGPTRKHYQPYAPPRDFAAYRSRVKFSRSA
DAPAYKQGQNQLYNELNLGRREEYDVLDKRRGRDPEMGGKPRRKNPQEGL
YNELQKDKMAEAYSEIGMKGERRRGKGHDGLYQGLSTATKDTYDALHMQA
LPPR.

Embodiment 105: An expression vector comprising the multicistronic expression system of any one of embodiments 1-104.
Embodiment 106: The expression vector of embodiment 105, wherein the expression vector comprises the polynucleotide of SEQ ID NO: 425, SEQ ID NO: 426, SEQ ID NO: 427, SEQ ID NO: 428, SEQ ID NO: 429, SEQ ID NO: 433, or SEQ ID NO: 432.
Embodiment 107: An isolated cell comprising the multicistronic expression system of any one of embodiments 1-104 or the expression vector of embodiment 105 or 106.
Embodiment 108: The isolated cell of embodiment 107, wherein the cell comprises an immune cell.
Embodiment 109: The isolated cell of embodiment 107, wherein the cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell.
Embodiment 110: The isolated cell of embodiment 107, wherein the cell is an NK cell.
Embodiment 111: A polynucleotide comprising the nucleic acid sequence of SEQ ID NO: 425, SEQ ID NO: 426, SEQ ID NO: 427, SEQ ID NO: 428, SEQ ID NO: 429, SEQ ID NO: 433, or SEQ ID NO: 432.
Embodiment 112: A polynucleotide comprising the nucleic acid sequence of SEQ ID NO: 425.
Embodiment 113: A polynucleotide comprising the nucleic acid sequence of SEQ ID NO: 426.
Embodiment 114: A polynucleotide comprising the nucleic acid sequence of SEQ ID NO: 427.
Embodiment 115: A polynucleotide comprising the nucleic acid sequence of SEQ ID NO: 428.
Embodiment 116: A polynucleotide comprising the nucleic acid sequence of SEQ ID NO: 429.
Embodiment 117: A polynucleotide comprising the nucleic acid sequence of SEQ ID NO: 432.
Embodiment 118: A method of treating a subject in need thereof, the method comprising administering a therapeutically effective dose of any of the isolated cells of any one of embodiments 107-110 or the polynucleotide of any one of embodiments 111-117, and 124.
Embodiment 119: A method of treating a subject with cancer, the method comprising administering to the subject an immunotherapy comprising of any of the isolated cells of any one of embodiments 107-110 or the polynucleotide of any one of embodiments 111-117, and 124.
Embodiment 120: A method of stimulating a cell-mediated immune response to a tumor cell in a subject, the method comprising administering to a subject having a tumor a therapeutically effective dose of any of the isolated cells of any one of embodiments 107-110 or the polynucleotide of any one of embodiments 111-117, and 124.
Embodiment 121: The method of any one of embodiments 111-120, wherein the isolated cell is derived from the subject.
Embodiment 122: The method of any one of embodiments 111-121, wherein the isolated cell is allogeneic with reference to the subject.
Embodiment 123: A method of making an engineered cell, comprising transducing an isolated cell with the multicistronic expression system of any one of embodiments 1-104, the expression vector of embodiment 105 or 106, or the polynucleotide of any one of embodiments 111-117, and 124.
Embodiment 124: A polynucleotide comprising the nucleic acid sequence of SEQ ID NO: 433.

### EXAMPLES

Below are examples of specific embodiments for carrying out the present invention. The examples are offered for illustrative purposes only, and are not intended to limit the scope of the present invention in any way. For example, the experiments described and performed below demonstrate the general utility of engineering cells to secrete payloads (e.g., effector molecules) and delivering those cells to induce an immunogenic response against tumors.

Efforts have been made to ensure accuracy with respect to numbers used (e.g., amounts, temperatures, etc.), but some experimental error and deviation should, of course, be allowed for.

### Example 1-FLT3 OR CD33 NOT EMCN Logic Gated CAR-NK Cell Circuit Optimization

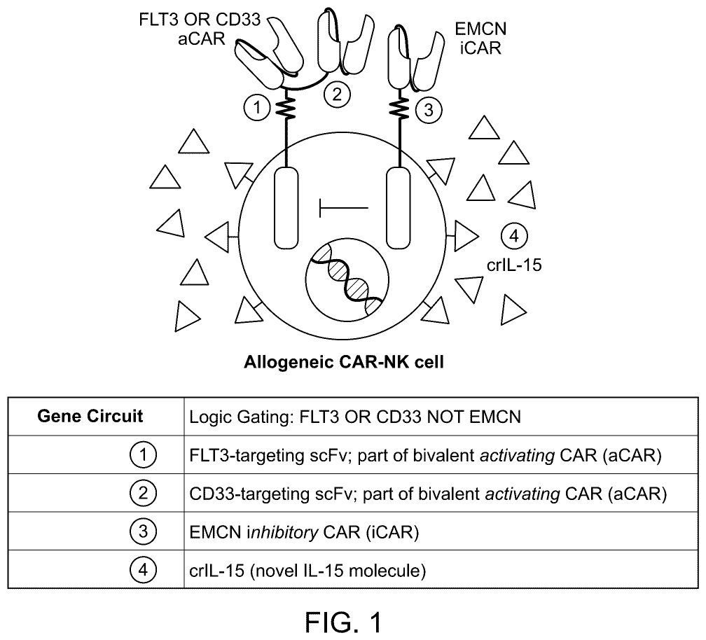

Various components, organization, and methods for FLT3 OR CD33 NOT EMCN logic-gated CAR-NK cell therapies were assessed. The overall FLT3 OR CD33 NOT EMCN logic-gated CAR-NK cell system is illustrated in FIG. 1, showing the bivalent activating CAR (aCAR) including a FLT3-targeting scFv and CD33-targeting scFv, the inhibitory CAR (iCAR) including an EMCN-targeting scFv, and a conditional-release IL-15 (crIL-15). The EMCN iCAR represents the “NOT-logic gate.” The bivalent FLT3/CD33 aCAR represents the “OR-logic gate.”

All the components were encoded in constructs in a multicistronic system where each of the components were encoded in a single engineered nucleic acid capable of transcription as a single mRNA transcript and separated by 2A ribosome skipping elements. Following transcription, each of the encoded proteins would be translated as distinct peptides.

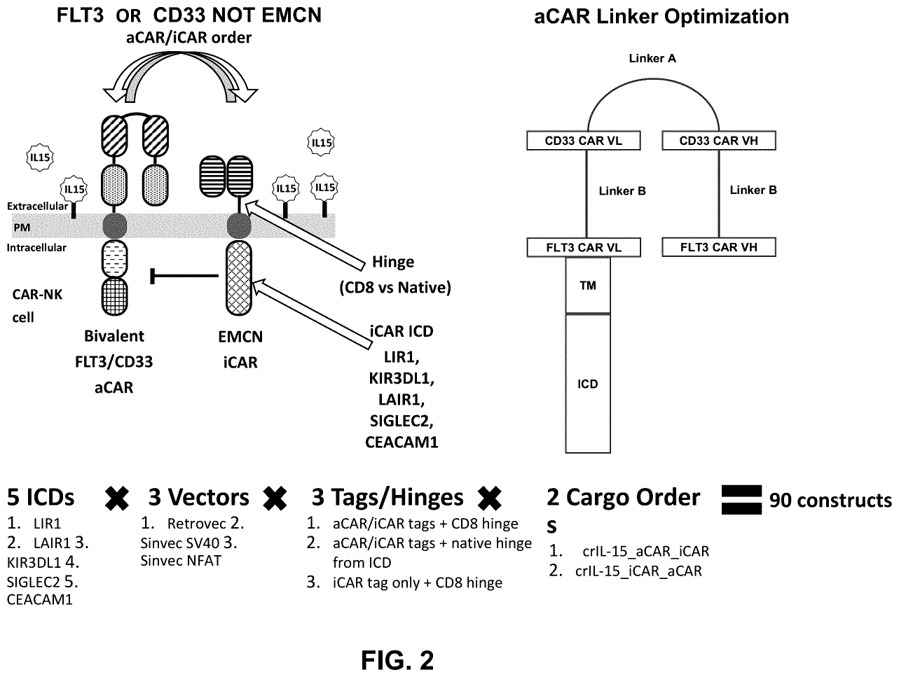

The various components and organizations that are assessed are shown in FIG. 2. Assessed were the order the EMCN iCAR and FLT3/CD33 bivalent aCAR were encoded, various inhibitory intracellular inhibitory domains, tag presence, various hinges, various linkers between antibody binding domains, and different retrovirus-based vectors. In total, the initial screen assessed 90 constructs.

### Methods

### Transduction for Construct Screening:

The following transduction procedure was used:
Add 1e6 NK cells/well
Titer: 1e10 genomic titer/1e6 cells or 1e9 genomic titer/1e6 cells
Serum-free transduction
NK MACS with IL-2 only
Spinoculate for 2 hours.
Rest cells after for 2+ hours before adding NK MACS+Human Ab serum+IL-2
Day 2: Transfer to 24-well G-Rex
Option 1 (Day 8): Check FLT3/CD33 and EMCN expression+IL-15 expression on surface and on Day 10 perform a cytotoxicity assay at a 1:1 E:T ratio (target cells=MV4-11+/−EMCN in IMDM media incubated for 18-20 hours)
Option 2 (Day 15): Check FLT3/CD33 and EMCN expression+IL-15 expression on surface and on Day 17 perform a cytotoxicity assay at a 1:2 E:T ratio (target cells=MV4-11+/−EMCN in IMDM media incubated for 18-20 hours)
Expression and target killing were assessed by flow cytometry

### Constructs:

The components and their order of the control constructs used are shown in Table A. The components and their order of the top constructs from the initial screen are shown in Table B.

TABLE A

| Controls | Construct | Vector |
|---|---|---|
| SB05438 | crIL15 E2A/T2A Myc bivalent | Retrovec |
| | loop6 aCAR E2A/T2A EMCN | |
| | iCAR | |
| SB05977 | crIL-15_ E2A/T2A _ Myc | Retrovec |
| | bivalent loop6 fitzer attas CD8 | |
| | hinge/TM CD28 ICD ”ALT | |
| | CD3z″_ E2A/T2A thermo fisher | |
| | codon optimized EMCN V5 LIR1 iCAR | |
| SB05439 | crIL15 E2A/T2A Myc CEA | Retrovec |
| | aCAR E2A/T2A EMCN iCAR | |

TABLE B

| SB# | Vector | Orientation | Tags | ICD |
|---|---|---|---|---|
| SB06461 | Sinvec | crIL-15-aCAR- | tags + native hinge | LIR-1 |
| | SV40 | iCAR | from ICD | |
| SB06462 | Sinvec | crIL-15-aCAR- | iCAR tag only + | LIR-1 |
| | SV40 | iCAR | CD8 | |
| SB06541 | Retrovec | crIL-15-aCAR- | iCAR tag only + | LIR-1 |
| | | aCAR | CD8 | |
| SB06467 | Retrovec | crIL-15-aCAR- | tags + native hinge | LIR-1 |
| | | iCAR | from ICD | |
| SB06433 | Sinvec | crIL-15-aCAR- | iCAR tag only + | KIR3DL1 |
| | SV40 | iCAR | CD8 | |
| SB06511 | Retrovec | crIL-15-iCAR- | tags + native hinge | KIR3DL1 |
| | | aCAR | from ICD | |
| SB06512 | Retrovec | crIL-15-iCAR- | iCAR tag only + | KIR3DL1 |
| | | aCAR | CD8 | |
| SB06447 | Sinvec | crIL-15-aCAR- | tags + native hinge | LAIR-1 |
| | SV40 | iCAR | from ICD | |
| SB06448 | Sinvec | crIL-15-aCAR- | iCAR tag only + | LAIR-1 |
| | SV40 | iCAR | CD8 | |
| SB06472 | Sinvec | crIL-15-aCAR- | iCAR tag only + | SIGLEC2 |
| | | iCAR | CD8 | |
| SB06557 | Retrovec | crIL-15-iCAR- | iCAR tag only + | SIGLEC2 |
| | | aCAR | CD8 | |
| SB06417 | Sinvec | crIL-15-aCAR- | tags + native hinge | CEACAM1 |
| | SV40 | iCAR | from ICD | |
| SB06487 | Sinvec | crIL-15-iCAR- | iCAR tag only + | CEACAM1 |
| | SV40 | aCAR | CD8 | |

### Component Sequences:

The component sequences of the assessed lead constructs are shown in Table C (Table C1 showing amino acid sequences and Table C2 showing nucleotide sequences).

TABLE C1

| Component | Amino Acid Sequence |
|---|---|
| IgGE | MDWTWILFLVAAATRVHS (SEQ ID NO: 228) |
| Signal | |
| Sequence | |
| I115 | NWVNVISDLKKIEDLIQSMHIDATLYTESDVHPSCKVTAMKCFLLELQVIS |
| | LESGDASIHDTVENLIILANNSLSSNGNVTESGCKECEELEEKNIKEFLQS |
| | FVHIVQMFINTS (SEQ ID NO: 224) |
| LR1 split | SGGGGSGGGGSGVTPEPIFSLIGGGSGGGGSGGGSLQ (SEQ ID NO: |
| N term | 250) |
| linker + | |
| Tace10 | |
| B7-1 TM | LLPSWAITLISVNGIFVICCLTYCFAPRCRERRRNERLRRESVRPV (SEQ |
| | ID NO: 204) |
| E2A/T2A | QCTNYALLKLAGDVESNPGPGSGEGRGSLLTCGDVEENPGP (SEQ ID |
| Ribosome | NO: 221) |
| Skipping | |
| Element | |
| GM- | MLLLVTSLLLCELPHPAFLLIP (SEQ ID NO: 423) |
| CSFRa | |
| Signal | |
| Sequence | |
| GGGGS | GGGGS (SEQ ID NO: 242) |
| Linker | |
| FLT3 VH | EVQLVQSGAEVKKPGSSVKVSCKASGGTFSSYAISWVRQAPGQGLEWMGGI |
| | IPIFGTANYAQKFQGRVTITADKSTSTAYMELSSLRSEDTAVYYCATFALF |
| | GFREQAFDIWGQGTTVTVSS (SEQ ID NO: 315) |
| CD33 VH | QVQLVQSGAEVKKPGSSVKVSCKASGYTFTDYNMHWVRQAPGQGLEWIGYI |
| | YPYNGGTGYNQKFKSKATITADESTNTAYMELSSLRSEDTAVYYCARGRPA |
| | MDYWGQGTLVTVSS (SEQ ID NO: 329) |
| 6 Loop | GSTSGSGKPGSGEGSTKG (SEQ ID NO: 247) |
| Linker | |
| CD33 VL | DIQMTQSPSSLSASVGDRVTITCRASESVDNYGISFMNWFQQKPGKAPKLL |
| | IYAASNQGSGVPSRFSGSGSGTDFTLTISSLQPDDFATYYCQQSKEVPWTF |
| | GQGTKVEIK (SEQ ID NO: 330) |
| FLT3 VL | DIQMTQSPSSLSASVGDRVTITCRASQSISSYLNWYQQKPGKAPKLLIYAA |
| | SSLQSGVPSRFSGSGSGTDFTLTISSLQPEDLATYYCQQSYSTPFTFGPGT |
| | KVDIK (SEQ ID NO: 316) |
| CD8 Hinge | ALSNSIMYFSHFVPVFLPAKPTTTPAPRPPTPAPTIASQPLSLRPEACRPA |
| #1 | AGGAVHTRGLDFACD (SEQ ID NO: 272) |
| CD8 TM | IYIWAPLAGTCGVLLLSLVITLYCNHR (SEQ ID NO: 206) |
| CD28 Co- | RSKRSRLLHSDYMNMTPRRPGPTRKHYQPYAPPRDFAAYRS (SEQ ID |
| Stim | NO: 287) |
| Domain | |
| CD3z ICD | RVKFSRSADAPAYKQGQNQLYNELNLGRREEYDVLDKRRGRDPEMGGKPRR |
| | KNPQEGLYNELQKDKMAEAYSEIGMKGERRRGKGHDGLYQGLSTATKDTYD |
| | ALHMQALPPR (SEQ ID NO: 289) |
| CD8 Signal | MALPVTALLLPLALLLHAARP (SEQ ID NO: 137) |
| Sequence | |
| EMCN VH | EVQLVESGGGLVQPGGSLRLSCAASGFTFSRYDMHWVRQAPGKGLEWVSVI |
| | WGNGNTHYHSALKSRFTISRDNSKNTLYLQMNSLRAEDTAVYYCTLRIKDW |
| | GQGTMVTVSS (SEQ ID NO: 302) |
| (GGGGS)3 | GGGGSGGGGSGGGGS (SEQ ID NO: 244) |
| linker | |
| EMCN VL | DVVMTQSPLSLPVTLGQPASISCKSSQSLVASDENTYLNWFQQRPGQSPRR |
| | LIYQVSKLDSGVPDRFSGSGSGTDFTLKISRVEAEDVGVYYCLQGIHLPWT |
| | FGQGTKLEIK (SEQ ID NO: 310) |
| CD8 Hinge | TTTPAPRPPTPAPTIALQPLSLRPEACRPAAGGAVHTRGLDFACD (SEQ |
| #2 | ID NO: 271) |
| LIR1 TM | VIGILVAVILLLLLLLLLFLI (SEQ ID NO: 259) |
| LIR1 ICD | LRHRRQGKHWTSTQRKADFQHPAGAVGPEPTDRGLQWRSSPAADAQEENLY |
| | AAVKHTQPEDGVEMDTRSPHDEDPQAVTYAEVKHSRPRREMASPPSPLSGE |
| | FLDTKDRQAEEDRQMDTEAAASEAPQDVTYAQLHSLTLRREATEPPPSQEG |
| | PSPAVPSIYATLAIH (SEQ ID NO: 285) |
| LIR1 | HPSDPLELVVSGPSGGPSSPTTGPTSTSGPEDQPLTPTGSDPQSGLGRHLG |
| Hinge | V (SEQ ID NO: 417) |
| (GGGGS)2 | GGGGSGGGGS (SEQ ID NO: 243) |
| Linker | |

TABLE C2

| Component | Nucleic Acid Sequence |
|---|---|
| IgGE | ATGGACTGGACTTGGATACTCTTTCTGGTCGCTGCCGCCACACGGGTG |
| Signal | CACTCT (SEQ ID NO: 229) |
| Sequence | |
| I115 | AATTGGGTCAACGTGATCAGCGACCTGAAGAAGATCGAGGACCTGATC |
| | CAGAGCATGCACATCGACGCCACACTGTACACCGAGTCCGATGTGCAC |
| | CCTAGCTGCAAAGTGACCGCCATGAAGTGCTTTCTGCTGGAACTGCAA |
| | GTGATCAGCCTGGAAAGCGGCGACGCCAGCATCCACGATACCGTGGAA |
| | AATCTGATCATCCTGGCCAACAACAGCCTGTCCAGCAACGGCAATGTG |
| | ACCGAGAGCGGCTGCAAAGAGTGCGAGGAACTGGAAGAGAAGAACATC |
| | AAAGAGTTTCTGCAGAGCTTCGTCCACATCGTGCAGATGTTCATCAAC |
| | ACCTCA (SEQ ID NO: 225) |
| LR1 split | TCAGGCGGCGGTGGTAGTGGAGGCGGAGGCTCAGGCGTGACCCCTGAG |
| N term | CCTATCTTCAGCCTGATCGGCGGAGGTTCCGGAGGTGGCGGTTCCGGC |
| linker + | GGAGGATCTCTTCAA (SEQ ID NO: 251) |
| Tace10 | |
| B7-1 TM | TTGCTGCCTAGCTGGGCCATCACACTGATCTCCGTGAACGGCATCTTC |
| | GTGATCTGCTGCCTGACCTACTGCTTCGCCCCTAGATGCAGAGAGCGG |
| | AGAAGAAACGAGCGGCTGAGAAGAGAAAGCGTGCGGCCTGTG (SEQ |
| | ID NO: 252) |
| E2A/T2A | CAGTGTACCAACTACGCCCTGCTGAAACTGGCCGGCGACGTGGAATCT |
| Ribosome | AATCCTGGACCTGGATCTGGCGAGGGACGCGGGAGTCTACTGACGTGT |
| Skipping | GGAGACGTGGAGGAAAACCCTGGACCT (SEQ ID NO: 222); or |
| Element | CAGTGCACAAATTATGCACTGCTGAAGCTCGCCGGGGATGTCGAGAGT |
| | AACCCAGGACCTGGAAGCGGAGAAGGTCGTGGTAGTCTACTAACGTGT |
| | GGTGATGTAGAAGAAAATCCTGGACCT (SEQ ID NO: 223) |
| GM- | ATGCTGCTGCTGGTTACATCTCTGCTGCTGTGCGAGCTGCCCCATCCT |
| CSFRa | GCCTTTCTGCTTATTCCT (SEQ ID NO: 424) |
| Signal | |
| Sequence | |
| GGGGS | GGCGGCGGTGGCTCT (SEQ ID NO: 254); or |
| (SEQ ID | GGTGGCGGCGGATCC (SEQ ID NO: 255) |
| NO: 242) | |
| Linker | |
| FLT3 VH | GAAGTTCAGCTGGTTCAGAGCGGAGCCGAAGTGAAGAAACCTGGCAGC |
| | AGCGTGAAGGTGTCCTGCAAAGCTTCTGGCGGCACCTTCAGCAGCTAC |
| | GCCATCTCTTGGGTTCGACAGGCACCTGGACAGGGCCTCGAGTGGATG |
| | GGAGGAATCATCCCTATCTTCGGCACCGCCAATTACGCCCAGAAATTT |
| | CAGGGAAGAGTGACCATCACCGCCGACAAGAGCACCAGCACCGCCTAC |
| | ATGGAACTGAGCAGCCTGAGAAGCGAGGATACCGCTGTGTATTACTGC |
| | GCCACATTCGCCCTGTTCGGCTTCAGAGAGCAGGCCTTCGATATCTGG |
| | GGCCAGGGCACAACCGTGACAGTTTCATCT (SEQ ID NO: 510) |
| CD33 VH | CAGGTTCAGCTTGTGCAATCTGGCGCTGAAGTGAAAAAGCCCGGCTCC |
| | TCCGTGAAAGTGTCTTGTAAAGCCAGCGGCTACACCTTTACCGACTAC |
| | AATATGCATTGGGTCCGCCAGGCACCAGGCCAGGGACTTGAATGGATC |
| | GGCTACATCTACCCCTACAACGGCGGCACCGGCTACAACCAGAAGTTC |
| | AAGTCCAAGGCCACAATCACAGCCGACGAGTCCACCAATACTGCTTAT |
| | ATGGAACTGTCTAGCCTTCGGAGCGAGGACACAGCCGTGTACTATTGC |
| | GCTAGAGGCAGACCCGCCATGGACTATTGGGGACAGGGAACCCTGGTC |
| | ACAGTGTCCAGC (SEQ ID NO: 511) |
| 6 Loop | GGCTCTACATCTGGCTCTGGCAAACCTGGAAGCGGCGAGGGATCTACC |
| Linker | AAGGGC (SEQ ID NO: 249) |
| CD33 VL | GACATCCAGATGACACAGAGCCCTAGTAGCCTGAGCGCCAGCGTGGGA |
| | GACAGAGTGACAATTACCTGTCGGGCCAGCGAGAGCGTGGACAACTAC |
| | GGCATCAGCTTCATGAACTGGTTCCAGCAGAAGCCTGGCAAGGCCCCT |
| | AAGCTGCTGATCTATGCCGCCAGCAATCAAGGCAGCGGAGTGCCCTCA |
| | AGATTCAGCGGAAGCGGCTCCGGCACCGACTTTACCCTGACAATCAGC |
| | TCCCTGCAGCCTGACGACTTCGCCACCTACTACTGCCAGCAGAGCAAA |
| | GAGGTGCCCTGGACATTCGGACAGGGCACCAAGGTGGAAATCAAA |
| | (SEQ ID NO: 512) |
| FLT3 VL | GATATTCAGATGACTCAGTCCCCAAGCAGCCTGTCAGCCTCCGTGGGC |
| | GATAGAGTCACCATTACATGCAGAGCCAGCCAGTCCATCTCCAGCTAC |
| | CTGAACTGGTATCAGCAAAAACCCGGAAAGGCTCCAAAGCTCCTCATC |
| | TACGCCGCTTCTAGCCTGCAGTCTGGCGTGCCATCTAGATTCTCTGGC |
| | TCCGGAAGCGGGACCGACTTCACACTGACCATATCTTCTCTGCAGCCC |
| | GAAGATCTGGCCACGTACTATTGTCAGCAGTCCTACAGCACCCCTTTC |
| | ACCTTCGGACCCGGAACAAAGGTGGACATTAAG (SEQ ID NO: |
| | 513) |
| CD8 Hinge | GCCCTGAGCAACAGCATCATGTACTTCAGCCACTTCGTGCCCGTGTTT |
| #1 | CTGCCCGCCAAGCCTACAACAACCCCTGCTCCTAGACCACCTACACCA |
| | GCTCCTACAATCGCCAGCCAGCCTCTGTCTCTGAGGCCCGAAGCTTGT |
| | AGACCAGCTGCTGGCGGAGCCGTGCATACAAGAGGACTGGATTTTGCC |
| | TGCGAC (SEQ ID NO: 284) |
| CD8 TM | ATCTATATCTGGGCCCCTCTGGCTGGCACATGCGGAGTTCTGCTGCTC |
| | AGCCTGGTCATCACCCTGTACTGCAACCACAGA (SEQ ID NO: |
| | 261) |
| CD28 Co- | AGAAGCAAGCGGAGCAGACTGCTGCACAGCGACTACATGAACATGACC |
| Stim | CCTAGACGGCCCGGACCTACCAGAAAGCACTACCAGCCTTACGCTCCT |
| Domain | CCTAGAGATTTCGCCGCCTACCGGTCC (SEQ ID NO: 288) |
| CD3z ICD | AGAGTGAAGTTCAGCAGGAGCGCAGACGCCCCCGCGTACAAGCAGGGC |
| | CAGAACCAGCTCTATAACGAGCTCAATCTAGGACGAAGAGAGGAGTAC |
| | GATGTTTTGGACAAGAGACGTGGCCGGGACCCTGAGATGGGGGGAAAG |
| | CCGAGAAGGAAGAACCCTCAGGAAGGCCTGTACAATGAACTGCAGAAA |
| | GATAAGATGGCGGAGGCCTACAGTGAGATTGGGATGAAAGGCGAGCGC |
| | CGGAGGGGCAAGGGGCACGATGGCCTTTACCAGGGTCTCAGTACAGCC |
| | ACCAAGGACACCTACGACGCCCTTCACATGCAGGCCCTGCCCCCTCGC |
| | (SEQ ID NO: 290) |
| CD8 Signal | ATGGCTCTGCCCGTGACAGCTTTGCTGCTGCCTTTGGCACTGCTGCTG |
| Sequence | CATGCTGCTAGACCA (SEQ ID NO: 416) |
| EMCN VH | GAGGTGCAGCTGGTTGAATCTGGCGGAGGACTGGTTCAGCCTGGCGGA |
| | TCTCTGAGACTGTCTTGTGCCGCCAGCGGCTTCACCTTCAGCAGATAC |
| | GATATGCACTGGGTCCGACAGGCCCCTGGCAAAGGACTTGAATGGGTG |
| | TCCGTGATCTGGGGCAACGGCAACACACACTACCACAGCGCCCTGAAG |
| | TCCCGGTTCACCATCTCCAGAGACAACAGCAAGAACACCCTGTACCTG |
| | CAGATGAACAGCCTGAGAGCCGAGGACACCGCCGTGTACTACTGCACC |
| | CTGAGAATCAAGGATTGGGGCCAGGGCACCATGGTCACCGTTTCTTCT |
| | (SEQ ID NO: 514) |
| (GGGGS)3 | GGAGGCGGAGGATCTGGTGGCGGAGGAAGTGGCGGAGGCGGTTCT |
| linker | (SEQ ID NO: 253) |
| (SEQ ID | |
| NO: 244) | |
| EMCN VL | GACGTGGTCATGACACAGAGCCCTCTGAGCCTGCCTGTGACACTGGGA |
| | CAGCCTGCCAGCATCAGCTGCAAGTCTAGCCAGTCTCTGGTGGCCAGC |
| | GACGAGAACACCTACCTGAACTGGTTCCAGCAGAGGCCCGGACAGTCT |
| | CCTAGACGGCTGATCTACCAGGTGTCCAAGCTGGATAGCGGCGTGCCC |
| | GATAGATTTTCTGGCAGCGGCTCTGGCACCGACTTCACCCTGAAGATC |
| | AGCAGAGTGGAAGCCGAGGACGTGGGCGTGTACTACTGTCTGCAAGGC |
| | ATCCATCTGCCTTGGACCTTTGGCCAGGGCACAAAGCTGGAAATCAAG |
| | (SEQ ID NO: 515) |
| CD8 Hinge | ACAACAACACCCGCACCTCGGCCTCCAACTCCAGCTCCAACAATTGCA |
| | CTGCAACCCCTGAGTCTGAGGCCCGAGGCCTGTAGGCCAGCAGCTGGC |
| | GGAGCTGTTCACACTAGAGGCCTGGACTTTGCCTGTGAC (SEQ ID |
| | NO: 283) |
| LIR1 TM | GTGATCGGCATTCTGGTCGCCGTGATCCTGCTCCTGTTGCTCCTGCTG |
| | CTTCTGTTCCTGATC (SEQ ID NO: 260) |
| LIR1 ICD | CTGCGGCACAGAAGGCAGGGCAAGCACTGGACAAGCACCCAGAGAAAG |
| | GCCGACTTTCAGCATCCTGCTGGCGCCGTTGGACCTGAGCCTACAGAT |
| | AGAGGACTGCAGTGGCGGTCTAGCCCTGCCGCTGATGCCCAAGAGGAA |
| | AATCTTTACGCCGCCGTGAAGCACACCCAGCCTGAGGATGGCGTGGAA |
| | ATGGACACCAGATCTCCCCACGATGAGGACCCTCAGGCCGTGACATAC |
| | GCAGAAGTGAAGCACTCCAGACCTCGGAGAGAGATGGCAAGCCCTCCA |
| | TCTCCTCTGAGCGGCGAGTTCCTGGACACCAAAGACAGACAGGCCGAA |
| | GAGGACAGACAGATGGATACCGAAGCCGCCGCTTCTGAAGCCCCACAG |
| | GATGTGACATATGCCCAGCTGCATAGCCTGACACTGCGGAGAGAAGCC |
| | ACAGAGCCTCCACCTTCTCAAGAAGGCCCATCTCCTGCCGTGCCTTCC |
| | ATCTATGCCACTCTGGCCATTCAC (SEQ ID NO: 286) |
| LIR1 Hinge | CACCCATCCGATCCTCTCGAGCTGGTGGTTTCTGGACCTTCTGGCGGC |
| | CCTAGCAGCCCTACAACAGGACCTACAAGCACAAGCGGCCCTGAGGAC |
| | CAACCTCTGACACCAACAGGCAGCGATCCTCAGTCTGGACTGGGGAGA |
| | CATCTGGGCGTT (SEQ ID NO: 418) |
| (GGGGS)2 | GGAGGCGGAGGATCTGGTGGTGGTGGATCT (SEQ ID NO: 256); |
| Linker | or |
| (SEQ ID | GGTGGCGGAGGAAGTGGCGGCGGAGGCTCT (SEQ ID NO: 257); |
| NO: 243) | or |
| | GGCGGTGGCGGATCTGGCGGAGGTGGCAGT (SEQ ID NO: 258) |

### CAR and crIL-15 Expression Assessment

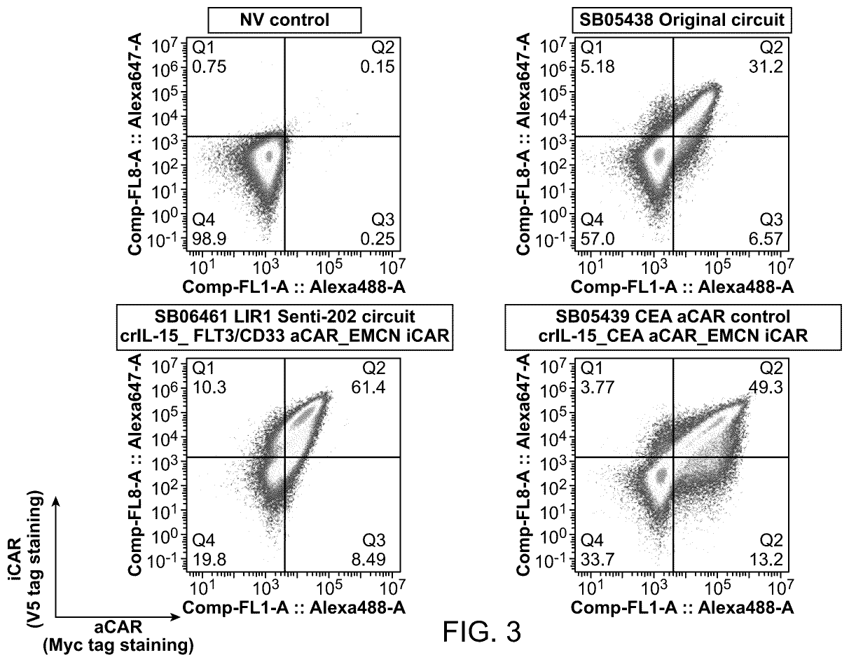

The expression of the EMCN iCAR, the FLT3/CD33 bivalent aCAR, and IL-15 were assessed for various constructs by flow cytometry. Each of the constructs in the screen had tags and used the “loop 6” linker. The gating strategy for iCAR and aCAR expression is shown in FIG. 3. The CEA control was used as a baseline.

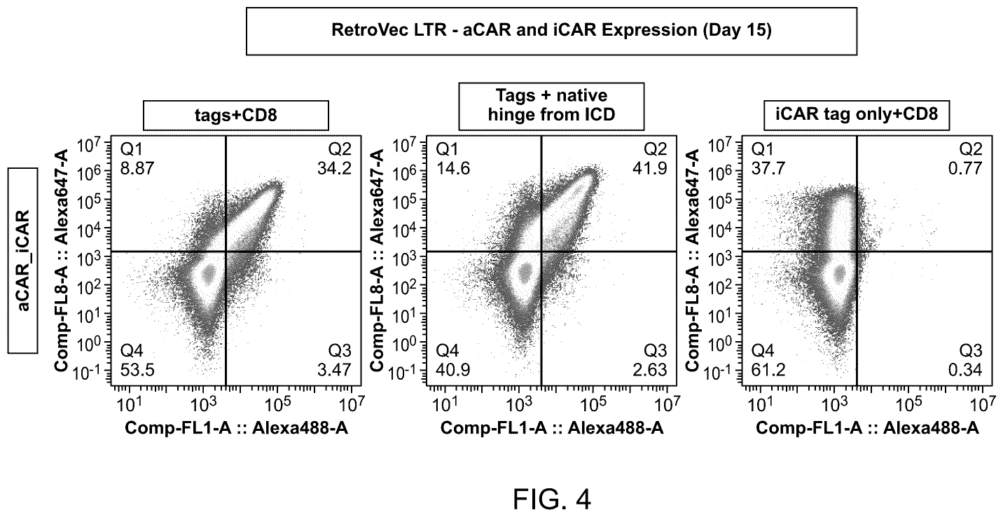

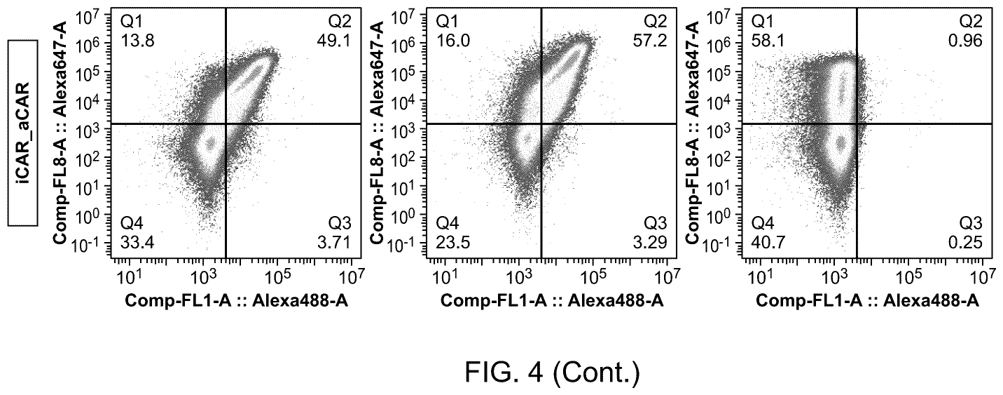

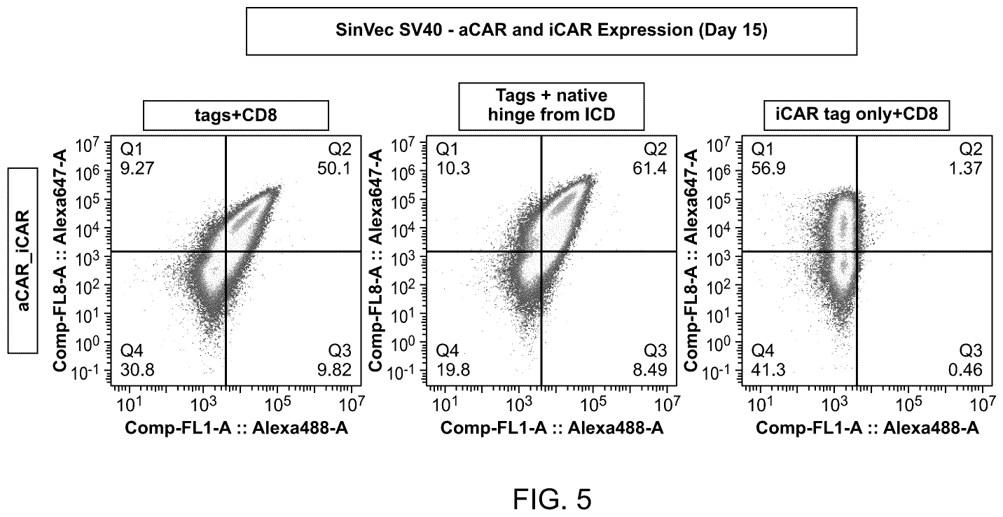

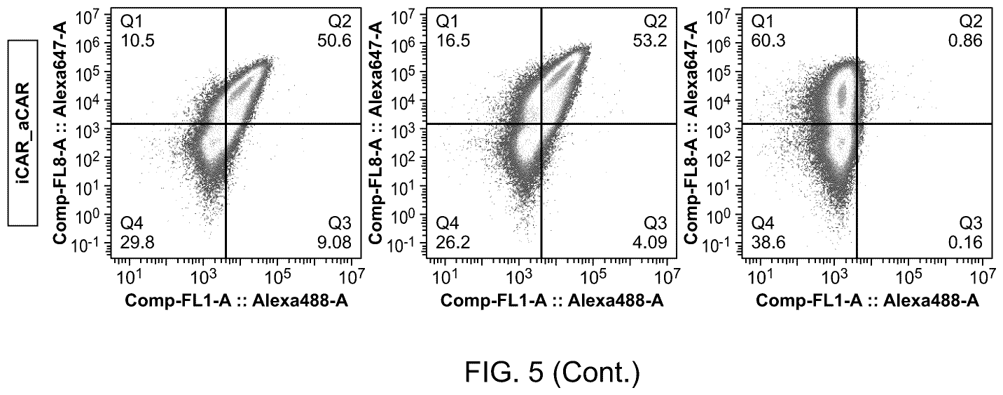

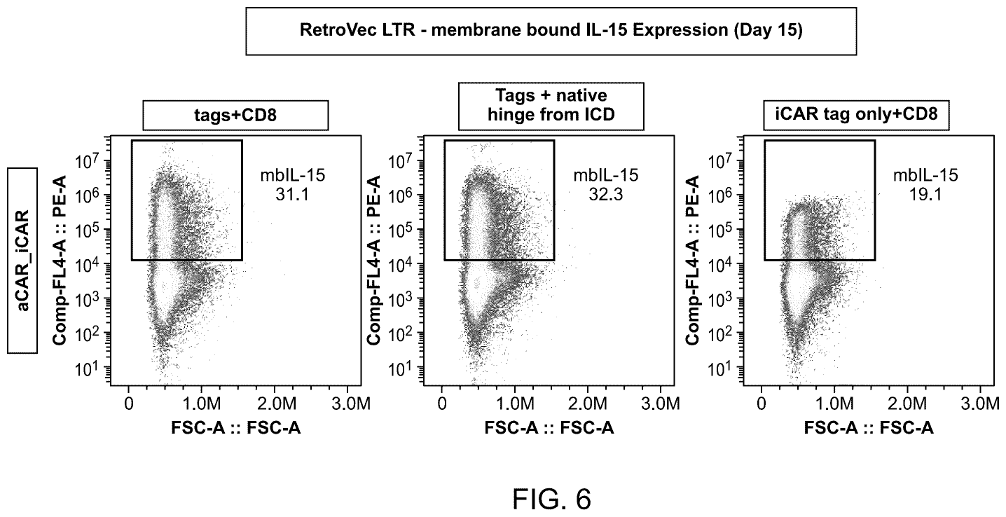

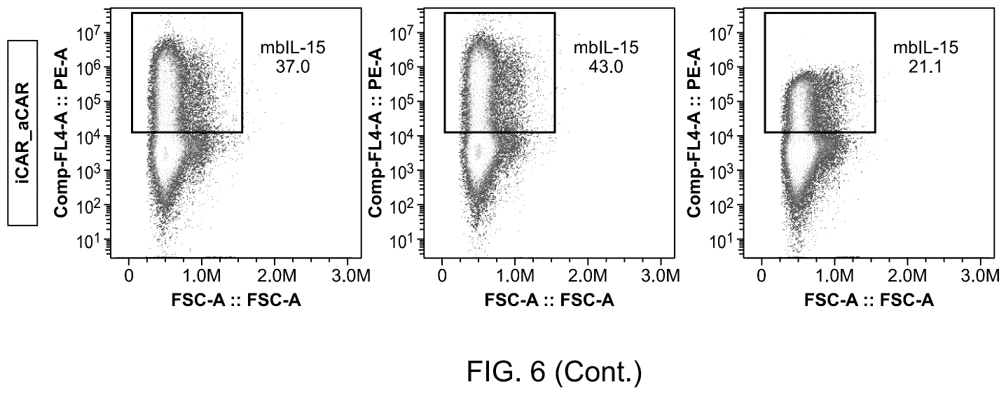

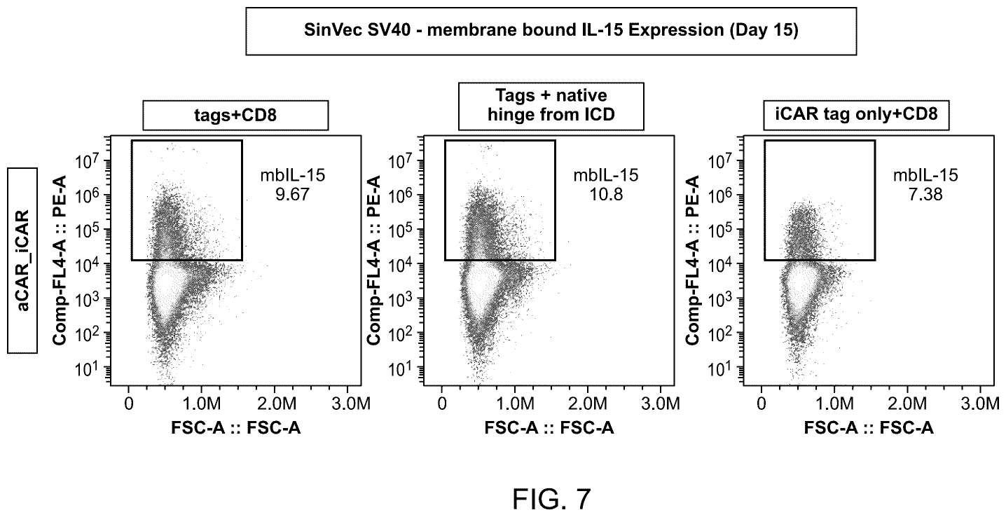

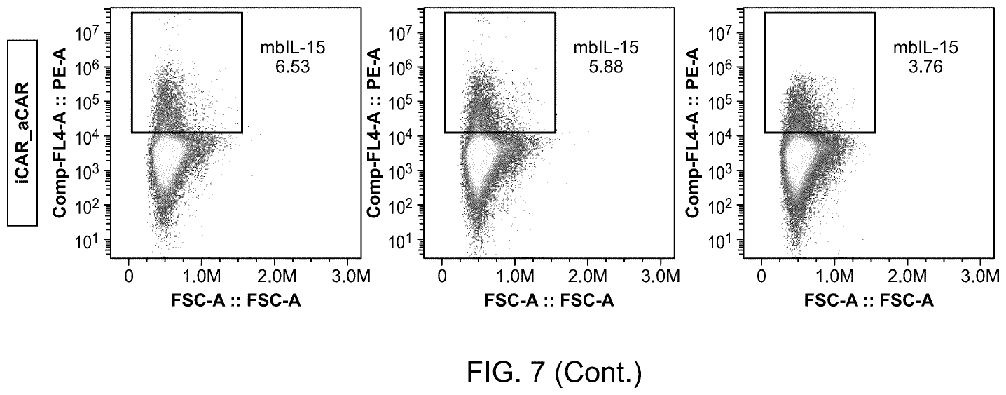

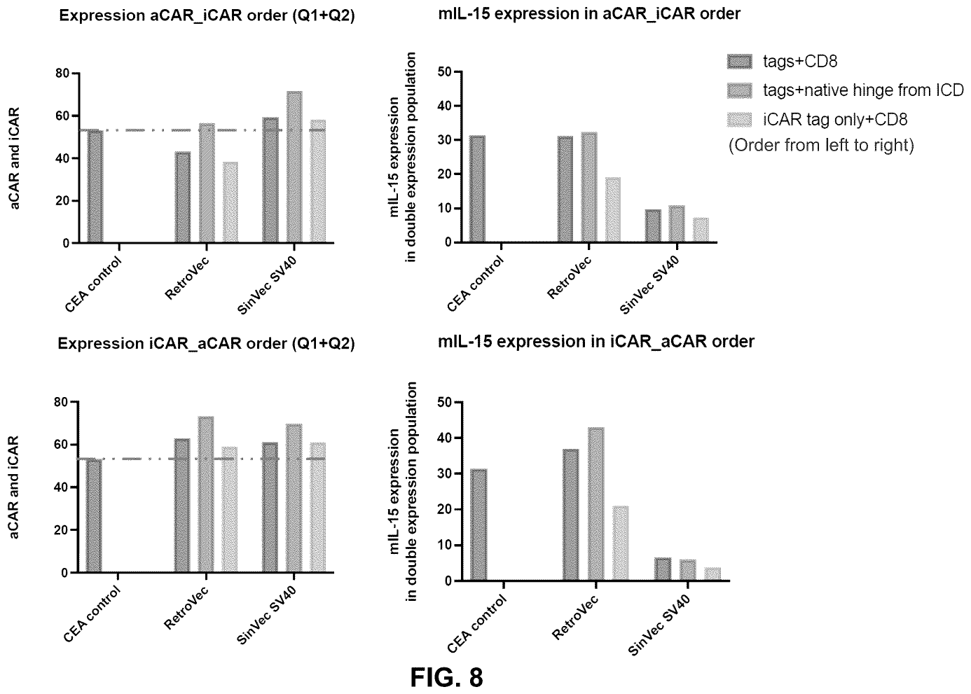

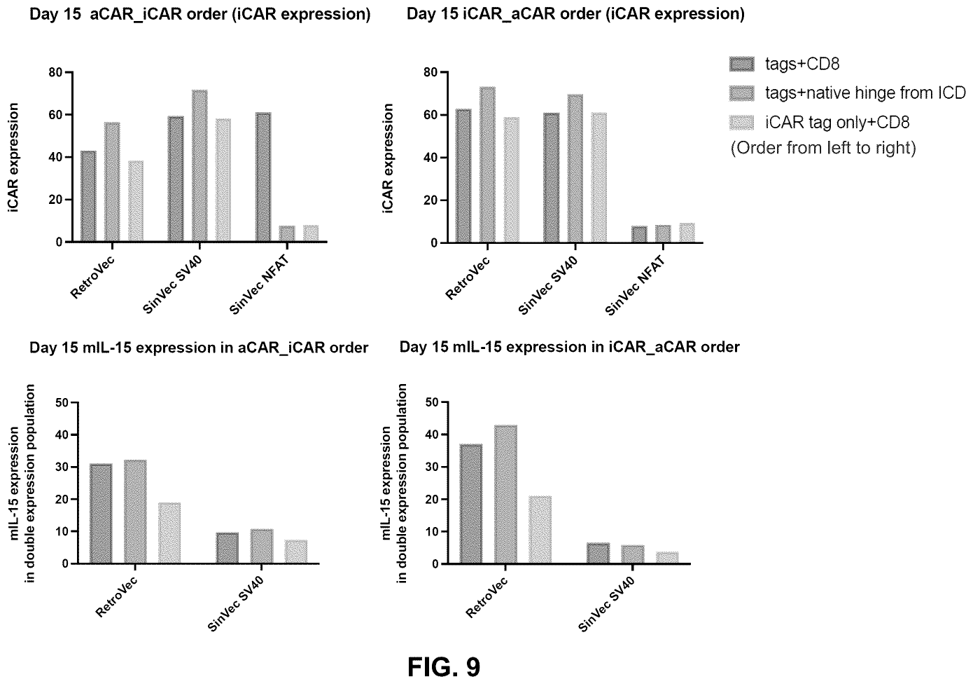

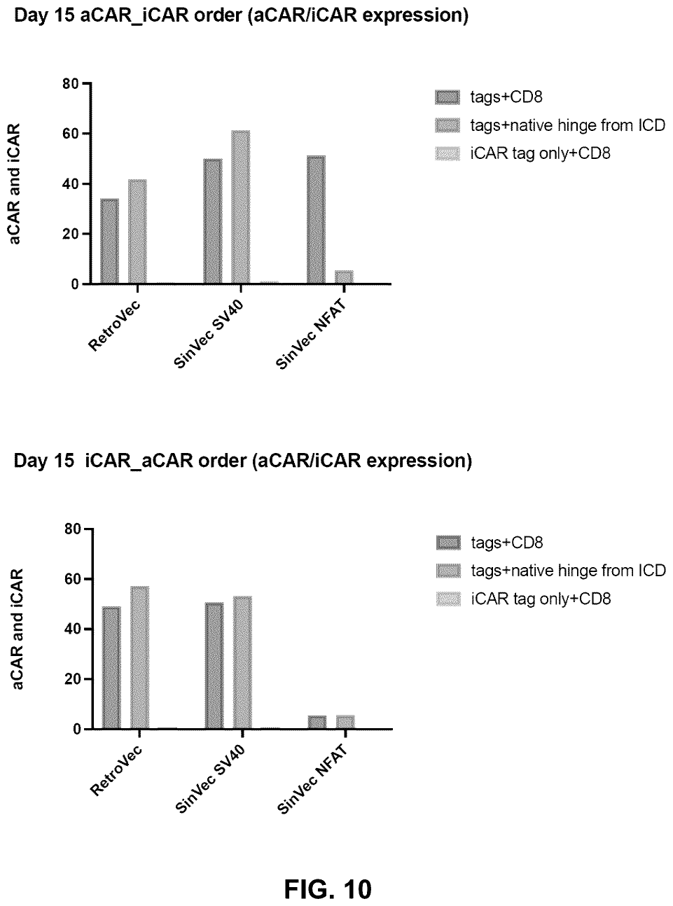

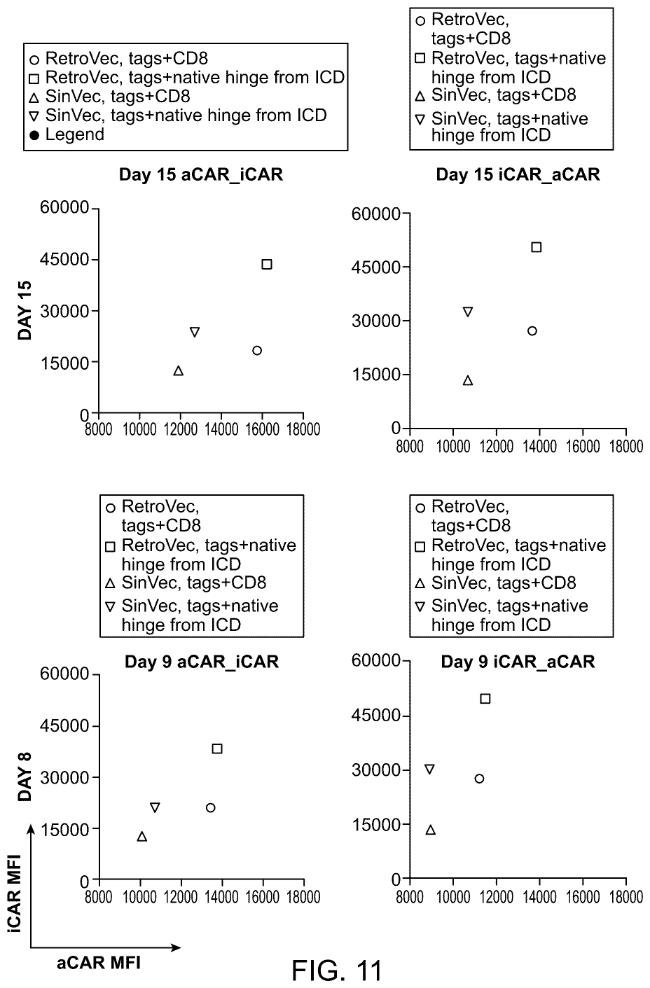

Representative flow cytometry plots are shown for aCAR and iCAR expression at Day 15 for a RetroVec (FIG. 4) and self-inactivated (SIN) γ-retroviral SINvec (FIG. 5) series of constructs. Representative flow cytometry plots are shown for membrane-bound IL-15 expression at Day 15 for a Retro Vec (FIG. 6) and SINvec (FIG. 7) series of constructs. A first series of comparisons for expression is shown in FIG. 8. A second series of comparisons for expression is shown in FIG. 9. A third series of comparisons for expression is shown in FIG. 10. A summary of aCAR and iCAR as assessed by MFI is shown in FIG. 11. Retrovec also had a higher virus yield than SINvec SV40 (data not shown).

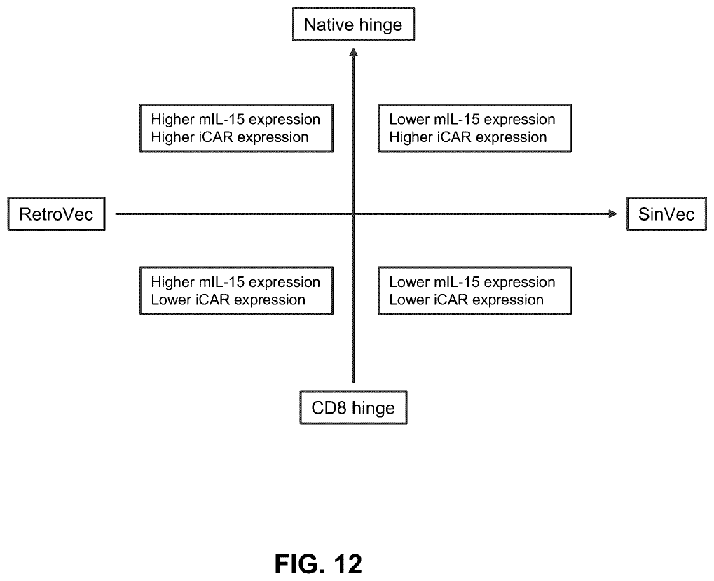

The results show at the same genomic titer MOI, the SINvec SV40 constructs demonstrated a similar CAR expression percentage as the Retrovec system while the MFI is lower. A different SINvec system (SINvec NFAT) generally had lower CAR expression. The results also show at the same genomic titer MOI, the SINvec SV40 constructs show lower membrane-bound IL-15 expression than the Retro Vec system. Expression generally trended higher for constructs with a native hinge from the intracellular domain (ICD) LIR1. A general summary of expression considerations for the various components and organizations are shown in FIG. 12, including that the different hinge and vector combinations may impact expression and/or impact in vitro/in vivo efficacy.

### Cytotoxicity Assessment

Cytotoxicity for the FLT3 OR CD33 NOT EMCN logic gate (e.g., CD33/FLT3 target killing and EMCN protection) was assessed for multicistronic systems expressing the EMCN iCAR, the FLT3/CD33 bivalent aCAR, and IL-15. Each of the constructs in the screen had tags and used the “loop 6” linker.

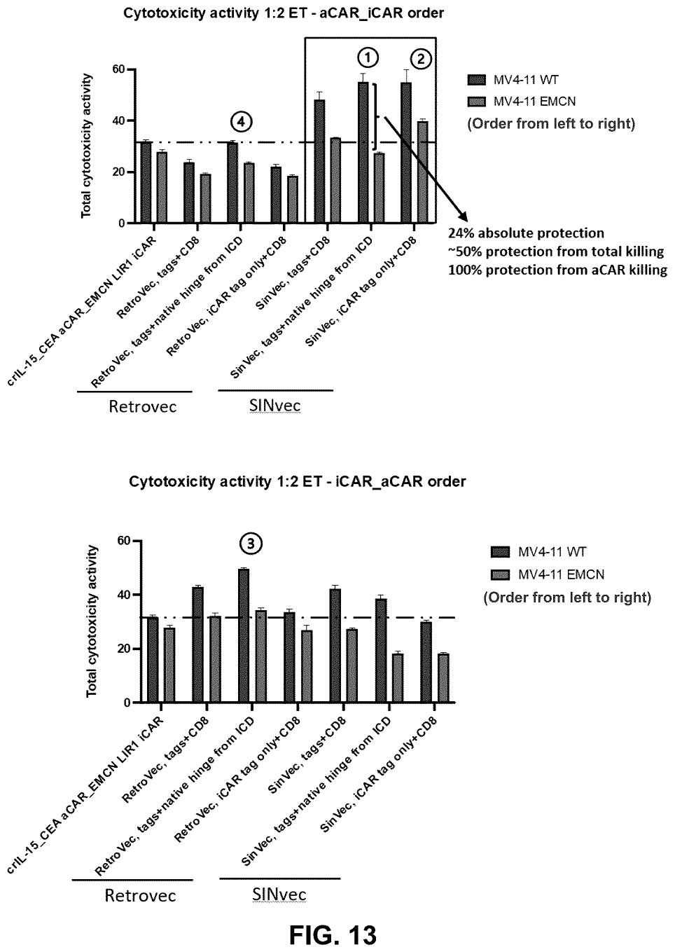

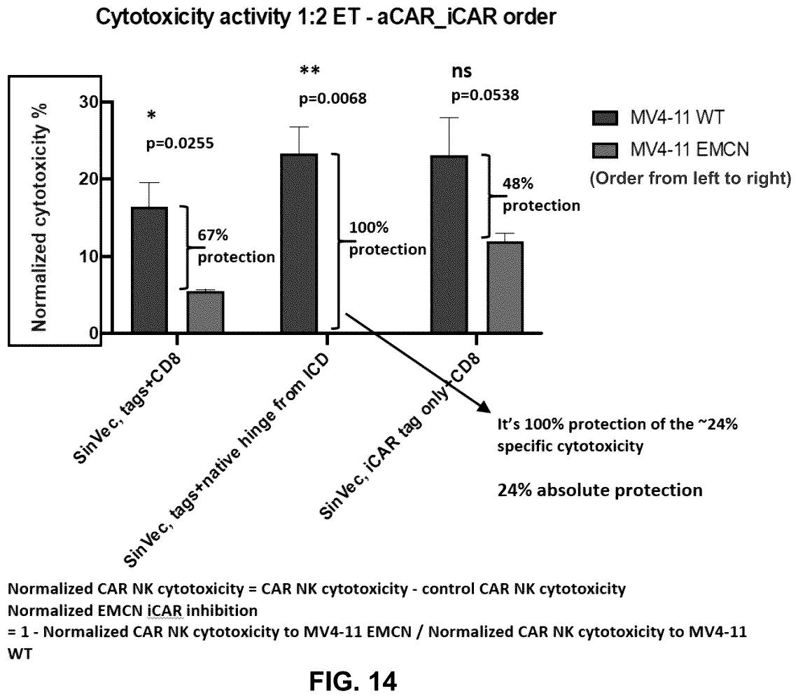

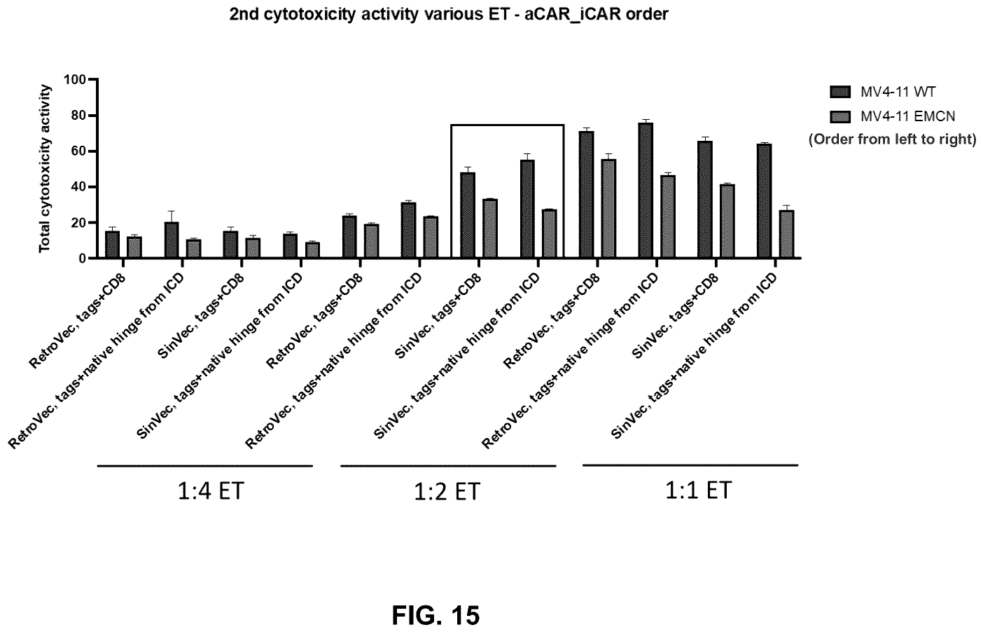

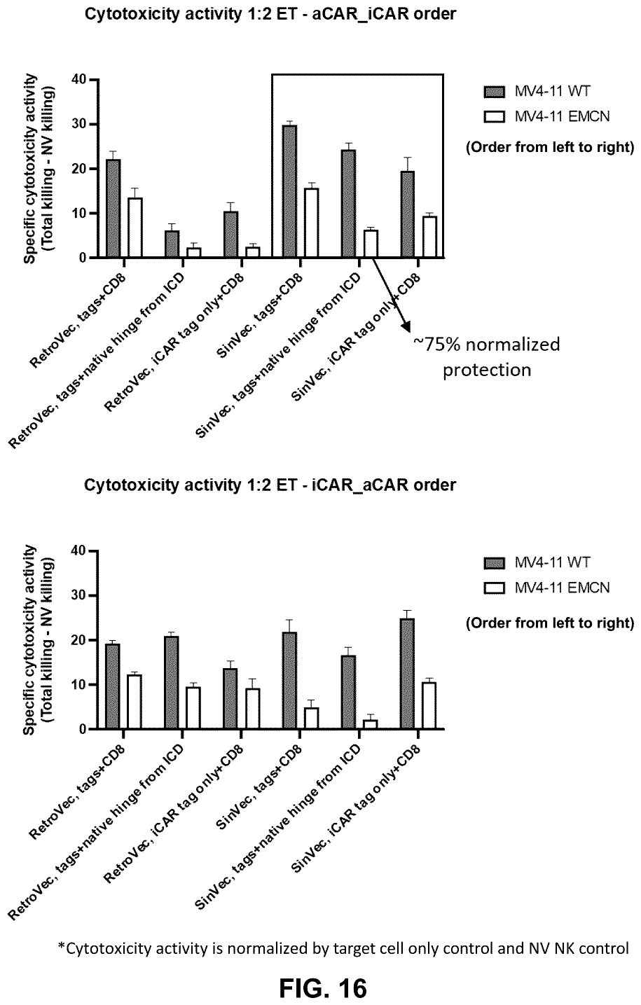

Total cytotoxicity against target cell lines expressing FLT3/CD33 (AML cell line MV4-11) and target cell lines engineered to also express the NOT gate target antigen EMCN (MV4-11+EMCN) for constructs using a LIR1 ICD is shown in FIG. 13. Normalized cytotoxicity was also assessed, as shown in FIG. 14. LIR1 constructs demonstrated up to 55% total killing (endogenous+aCAR-mediated killing). The LIR1 constructs also demonstrated up to ˜50% protection from total killing (24% absolute protection; 100% protection of aCAR-mediated killing). As shown in FIG. 15, cytotoxicity was also assessed at Day 17 post-transduction for different effector:target ratios and demonstrated good cytotoxicity profiles at 1:2 and 1:4 ratios. As shown in FIG. 16, normalized cytotoxicity was also assessed at Day 10 post-transduction at a 1:2 effector:target ratio for the various LIR1-ICD constructs and demonstrated up to 18% absolute protection. The cytotoxicity profile indicate constructs with a native hinge from the intracellular domain (ICD) LIR1 generally worked best.

### Example 2-CAR-NK Cell Assessment in Mouse Models

Assessed were bivalent FLT3 OR CD33 CAR-NK cells in comparison to monospecific CAR-NK cells in mouse models. Also assessed were aCARs using either “loop 4” or “loop 6” linkers sequences.

### Methods

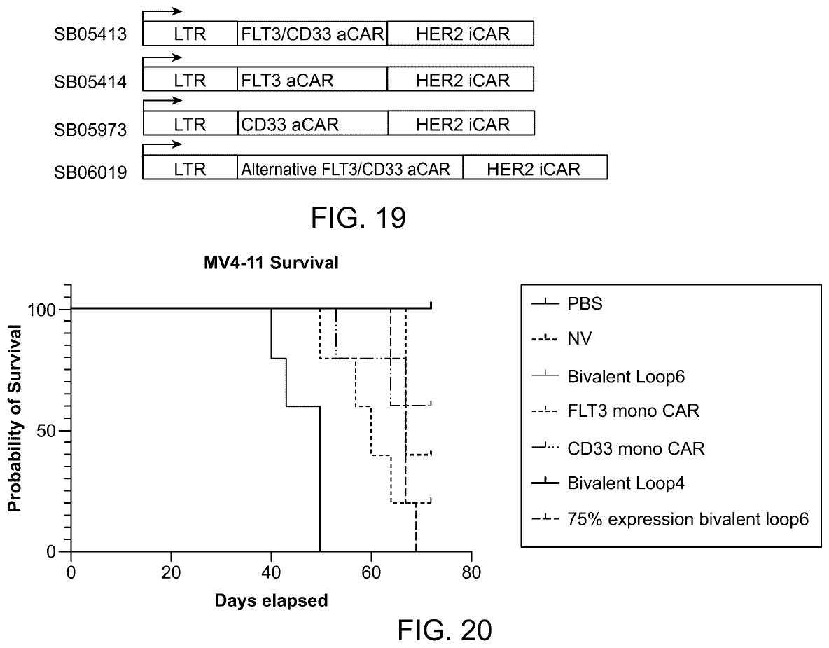

The components and their order of the constructs are shown in Table D. The organization of the CAR constructs is shown in FIG. 19. A total of 35e6 NK cells were administered as a single CAR-NK injection with 5 mice per group. CAR NK cells were pre-mixed with tumor cells prior to injection. All constructs were RetroVec vector systems. Target cells (tumor models) assessed were: (1) MV4-11 [FLT3 expression high; CD33 expression high]; (2) Molm13 [FLT3 expression medium; CD33 expression high]; and SEM [FLT3 expression high; CD33 expression low].

TABLE D

| | | | Target cells and |
|---|---|---|---|
| Group | SB # | Components | potential killing level |
| 1 | None | Tumor cell only | MV4-11, No killing |
| 2 | None | Unengineered NK cells | MV4-11, |
| | | | Basal killing |
| 3 | SB05413 | FLT3/CD33 bivalent | MV4-11, High |
| | | loop6 aCAR_HER2 | |
| | | iCAR | |
| 4 | SB05414 | FLT3 aCAR_dual | MV4-11, Medium- |
| | | 2A_HER2 iCAR | High |
| 5 | SB05973 | CD33 aCAR_dual | MV4-11, Medium- |
| | | 2A_HER2 iCAR | High |
| 6 | SB06019 | FLT3/CD33 bivalent | MV4-11, High |
| | | loop4 aCAR_HER2 | |
| | | iCAR | |
| 7 | SB05413 | Transduction titration | MV4-11, High |
| | | for bivalent loop6 | |

### Component Sequences:

The component sequence of the assessed constructs are shown in Table C in Example 1.

### Bivalent FLT3 OR CD33 NK CAR Vs Monospecific NK CAR Assessment

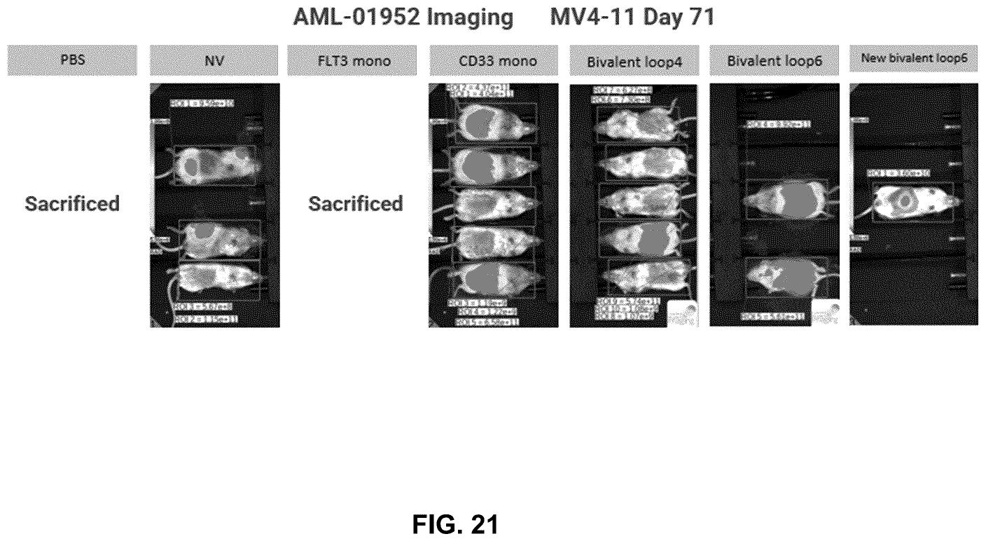

Bivalent FLT3 OR CD33 CAR-NK cells and monospecific CAR-NK cells were assessed in MV4-11 murine tumor models. Survival is shown in FIG. 20. Representative tumor imaging is shown in FIG. 21. The results show that CARs with the loop 4 linker demonstrated better efficacy than CARs with the loop 4 linker. The order for efficacy (starting with the best) was determined to be: (1) Bivalent Loop4 CAR, (2) CD33 mono CAR, (3) Bivalent Loop4 CAR, (4) FLT3 mono CAR. Also noted is the NV NK group had higher basal killing observed than in previous experiments (data not shown). The results demonstrate that the bivalent loop4 CAR NK had the best anti-tumor efficacy and survival rate among all the groups and had similar/better efficacy as compared to the mono CAR approach.

### Example 3—FLT3 OR CD33 NOT EMCN CAR-NK Cell Further Optimization

Various components, organization, and methods for FLT3 OR CD33 NOT EMCN logic-gated CAR-NK cell therapies were further assessed. As above, all the components were encoded in constructs in a multicistronic system.

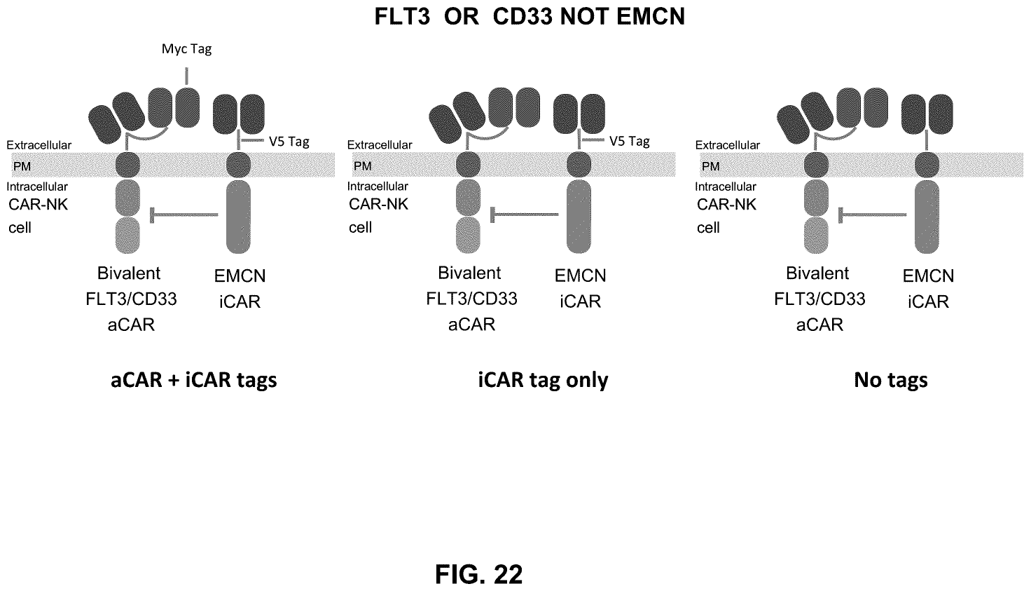

Assessed were the order the EMCN iCAR and FLT3/CD33 bivalent aCAR were encoded, various inhibitory intracellular inhibitory domains, tag presence, various hinges, various linkers between antibody binding domains, and different retrovirus-based vectors. Specifically assessed were constructs that in part varied whether CARs included affinity tags or were “tagless” versions, as well as the linker “loop 4” used between the variable domains of the bivalent aCAR. FIG. 22 illustrates the various tagged and tagless combinations assessed. The additional candidates assessed are shown in Table E. Also assessed were the top candidates from Example 1, as shown in Table F. Controls were the same as those in Example 1.

### Methods

### Transduction For Construct Screening:

The following transduction procedure was used (Days refer Days Post-Transduction):
Add 1e6 NK cells/well
Titer: 3e10 genomic titer/1e6 cells or 2e10 genomic titer/1e6 cells
Serum-free transduction
NK MACS with IL-2 only
Spinoculate for 2 hours
Rest cells after for 2+ hours before adding NK MACS+Human Ab serum+IL-2.
Day 2: Transfer to 24-well G-Rex
Day 8: Check FLT3/CD33 and EMCN expression+IL-15 expression on surface and on Day 9-10 perform a cytotoxicity assay at a 1:2 E:T ratio or 1:4 E:T ratio (target cells=MV4-11+/−EMCN in IMDM media incubated for 18-20 hours)
Expression and target killing were assessed by flow cytometry

TABLE E

| SB # | Construct | Vector | Tags |
|---|---|---|---|
| SB07401 | crIL-15_aCAR_iCAR | Sinvec | no tags + |
| | orientation:aCAR scfv- | SV40 | CD8 |
| | loop 4 humanized EMCN | | |
| | LIR1 iCAR tags/hinge | | |
| SB07403 | crIL-15_aCAR_iCAR | Retrovec | no tags + CD8 |
| | orientation:aCAR scfv- | | |
| | loop 4 humanized EMCN | | |
| | LIR1 iCAR tags/hinge | | |
| SB07405 | crIL-15_aCAR_iCAR | Sinvec | no tags + |
| | orientation:aCAR scfv- | SV40 | native hinge |
| | loop 4 humanized EMCN | | from ICD |
| | LIR1 iCAR tags/hinge | | |
| SB07404 | crIL-15_aCAR_iCAR | Sinvec | iCAR tag |
| | orientation:aCAR scfv- | SV40 | only + CD8 |
| | loop 4 humanized EMCN | | |
| | LIR1 iCAR tags/hinge | | |
| SB07408 | crIL-15_aCAR_iCAR | Retrovec | iCAR tag |
| | orientation:aCAR scfv- | | only + CD8 |
| | loop 4 humanized EMCN | | |
| | LIR1 iCAR tags/hinge | | |
| SB07409 | crIL-15_aCAR_iCAR | Retrovec | no tags + |
| | orientation:aCAR scfv- | | native hinge |
| | loop 4 humanized EMCN | | from ICD |
| | LIR1 iCAR tags/hinge | | |
| SB07410 | crIL15_iCAR_aCAR | Sinvec | iCAR tag |
| | orientation:aCAR scfv- | SV40 | only + CD8 |
| | loop 4 humanized EMCN | | |
| | LIR1 iCAR tags/hinge | | |
| SB07412 | crIL15_iCAR_aCAR | Sinvec | no tags + |
| | orientation:aCAR scfv- | SV40 | native hinge |
| | loop 4 humanized EMCN | | from ICD |
| | LIR1 iCAR tags/hinge | | |
| SB07416 | crIL15_iCAR_aCAR | Retrovec | iCAR tag |
| | orientation:aCAR scfv- | | only + CD8 |
| | loop 4 humanized EMCN | | |
| | LIR1 iCAR tags/hinge | | |
| SB07417 | crIL15_iCAR_aCAR | Retrovec | no tags + CD8 |
| | orientation:aCAR scfv- | | |
| | loop 4 humanized EMCN | | |
| | LIR1 iCAR tags/hinge | | |
| SB07418 | crIL15_iCAR_aCAR | Retrovec | no tags + native |
| | orientation:aCAR scfv- | | hinge |
| | loop 4 humanized EMCN | | from ICD |
| | LIR1 iCAR tags/hinge | | |

TABLE F

| SB # | Construct | Vector | Tags |
|---|---|---|---|
| SB06511 | crIL-15-iCAR-aCAR, | Retrovec | tags + native |
| | loop 6, KIR3DL1 | | hinge from ICD |
| SB06512 | crIL-15-aCAR-iCAR, | Retrovec | iCAR tag only + CD8 |
| | loop 6, KIR3DL1 | | |
| SB06557 | crIL-15-iCAR-aCAR | Retrovec | iCAR tag only + CD8 |
| | loop 6, SIGLEC2 | | |
| SB06541 | crIL-15-iCAR-aCAR | Retrovec | tags + native |
| | loop 6, LIR1 | | hinge from ICD |
| SB06467 | crIL-15-aCAR-iCAR, | Retrovec | tags + native |
| | loop 6, LIR1 | | hinge from ICD |

### Component Sequences:

The component sequence of the assessed constructs are shown in Table C in Example 1. Lead candidates and their domain organizations are:
SB06342; SINvec SV40 (full vector sequence is SEQ ID NO: 425)
   IgGE SS--Il15--LR1 split N term linker+Tace10--B7-1 TM--E2A/T2A--GM-CSFRa SS--FLT3 VH-GGGGS (SEQ ID NO: 242)--CD33 VH--6 Loop (GSTSGSGKPGSGEGSTKG) (SEQ ID NO: 247)--CD33 VL-GGGGS (SEQ ID NO: 242)--FLT3 VL--CD8 Hinge--CD8 TM--CD28--CD3z--E2A/T2A--CD8 SS--EMCN VH--(GGGGS) 3 (SEQ ID NO: 244) linker--EMCN VL--CD8 Hinge--LIR1 TM--LIR1 ICD
SB06463; SINvec SV40 (full vector sequence is SEQ ID NO: 426)
   IgGE SS--Il15--LR1 split N term linker+Tace10--B7-1 TM--E2A/T2A--GM-CSFRa SS--FLT3 VH-GGGGS (SEQ ID NO: 242)--CD33 VH--6 Loop (GSTSGSGKPGSGEGSTKG) (SEQ ID NO: 247)--CD33 VL--GGGGS (SEQ ID NO: 242)--FLT3 VL--CD8 Hinge--CD8 TM--CD28--CD3z--E2A/T2A--CD8 SS--EMCN VH--(GGGGS) 3 (SEQ ID NO: 244 linker--EMCN VL-LIR1 Hinge--LIR1™--LIR1 ICD
SB07401; SINvec SV40 (full vector sequence is SEQ ID NO: 427)
   IgGE SS--1115--LR1 split N term linker+Tace10--B7-1 TM--E2A/T2A--GM-CSFRa SS--FLT3 VH-(GGGGS) 2 (SEQ ID NO: 2423 linker--CD33 VH--(GGGGS) 2 (SEQ ID NO: 2423 linker--CD33 VL--(GGGGS) 2 (SEQ ID NO: 2423 linker--FLT3 VL--CD8 Hinge--CD8 TM--CD28--CD3z--E2A/T2A--CD8 SS--EMCN VH--(GGGGS) 3 (SEQ ID NO: 2424 linker--EMCN VL-CD8 Hinge--LIR1 TM--LIR1 ICD
SB07405; SINvec SV40 (full vector sequence is SEQ ID NO: 428)
   IgGE SS--Il15--LR1 split N term linker+Tace10--B7-1 TM--E2A/T2A--GM-CSFRa SS--FLT3 VH-(GGGGS) 2 (SEQ ID NO: 243) linker--CD33 VH--(GGGGS) 2 (SEQ ID NO: 243) linker--CD33 VL--(GGGGS) 2 (SEQ ID NO: 243) linker--FLT3 VL--CD8 Hinge--CD8 TM--CD28--CD3z--E2A/T2A--CD8 SS--EMCN VH--(GGGGS) 3 (SEQ ID NO: 244) linker--EMCN VL-LIR1 Hinge--LIR1TM--LIR1 ICD
SB07412; SINvec SV40 (full vector sequence is SEQ ID NO: 429)
   IgGE SS--Il15--LR1 split N term linker+Tace10--B7-1 TM--E2A/T2A--CD8 SS--EMCN VH--(GGGGS) 3 (SEQ ID NO: 244) linker--EMCN VL-LIR1 Hinge--LIR1 TM--LIR1 ICD--E2A/T2A--GM-CSFRa SS--FLT3 VH-(GGGGS) 2 (SEQ ID NO: 243) linker--CD33 VH--(GGGGS) 2 (SEQ ID NO: 243) linker--CD33 VL--(GGGGS) 2 (SEQ ID NO: 243) linker--FLT3 VL--CD8 Hinge--CD8 TM--CD28--CD3z

### Flow Cytometry of Tagless Constructs:

Fluorophore/biotin-conjugated antigen for CD33, FLT3, and EMCN CARs was added to CAR NK cells. CD33 and EMCN were run in same panel and FLT3 run in separate panel.

### Tagged and Tagless Expression Assessment

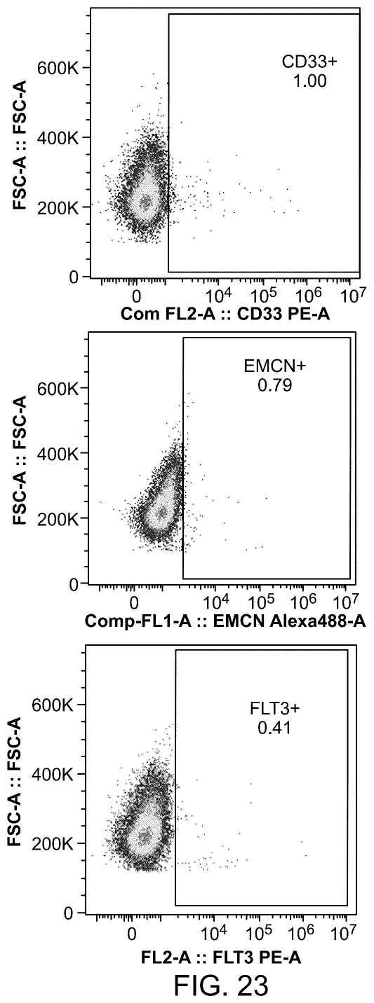

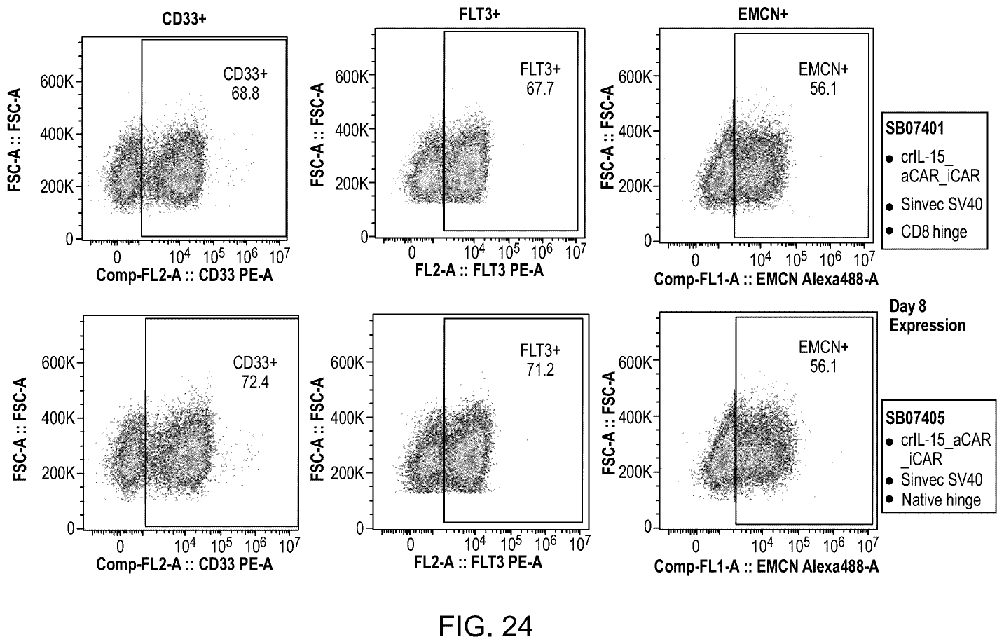

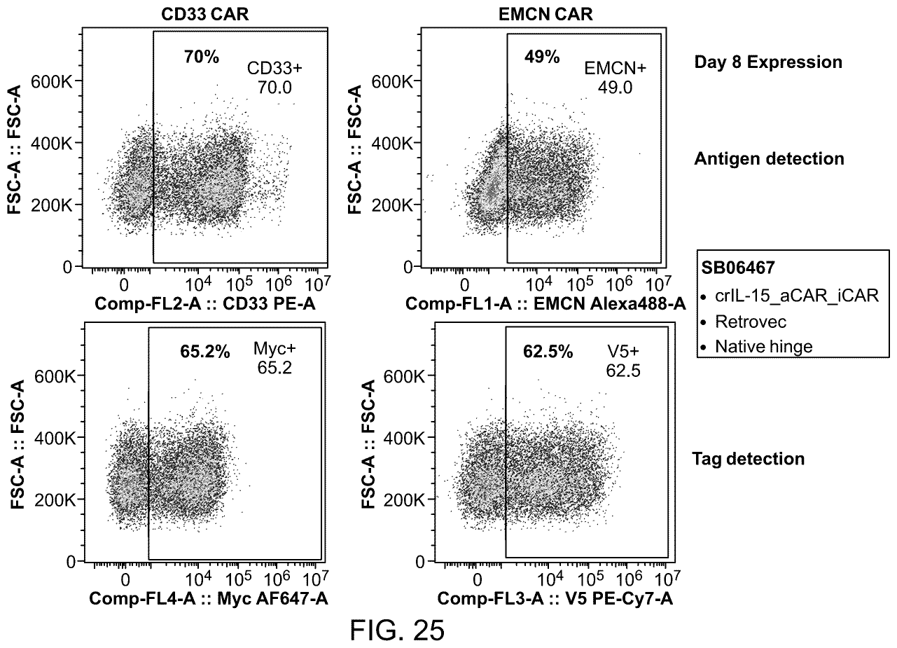

The expression of the tagless versions of the EMCN iCAR and the FLT3/CD33 bivalent aCAR were assessed for various constructs by flow cytometry. Staining of the “no virus” control is shown in FIG. 23. Staining of tagless constructs SB07401 and SB07405 is shown in FIG. 24. Staining of iCAR tagged only construct SB06467 is shown in FIG. 25. The results demonstrate “tagless” CARs could be detected using FLT3, CD33, and EMCN fluorophore-conjugated antigens expression was similar to tagged CAR versions.

### Loop 4 and Loop 6 Expression Assessment

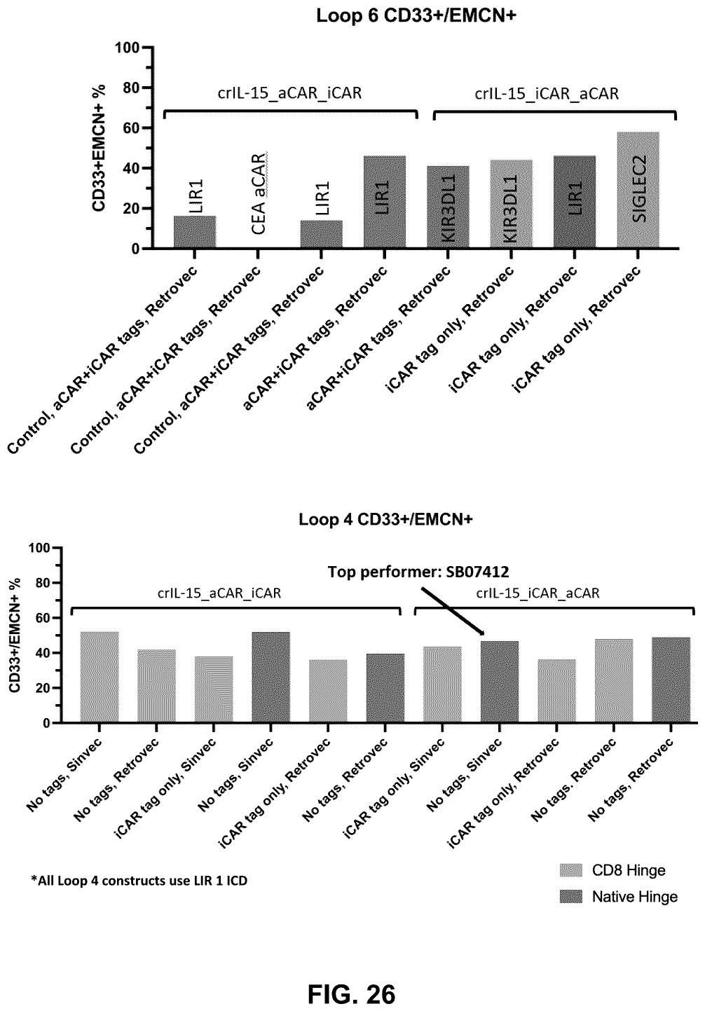

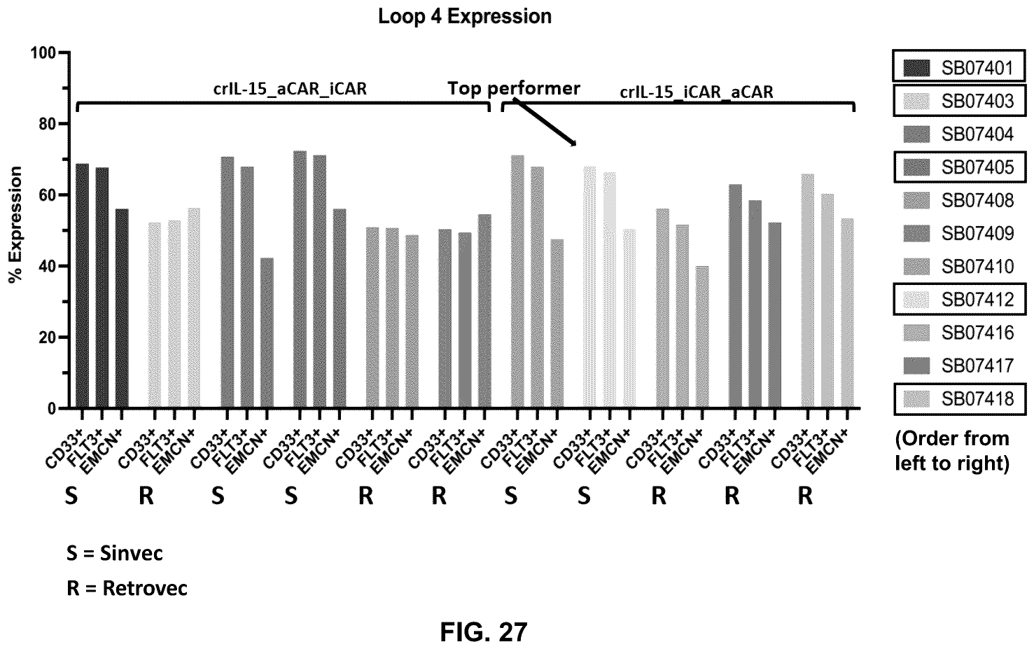

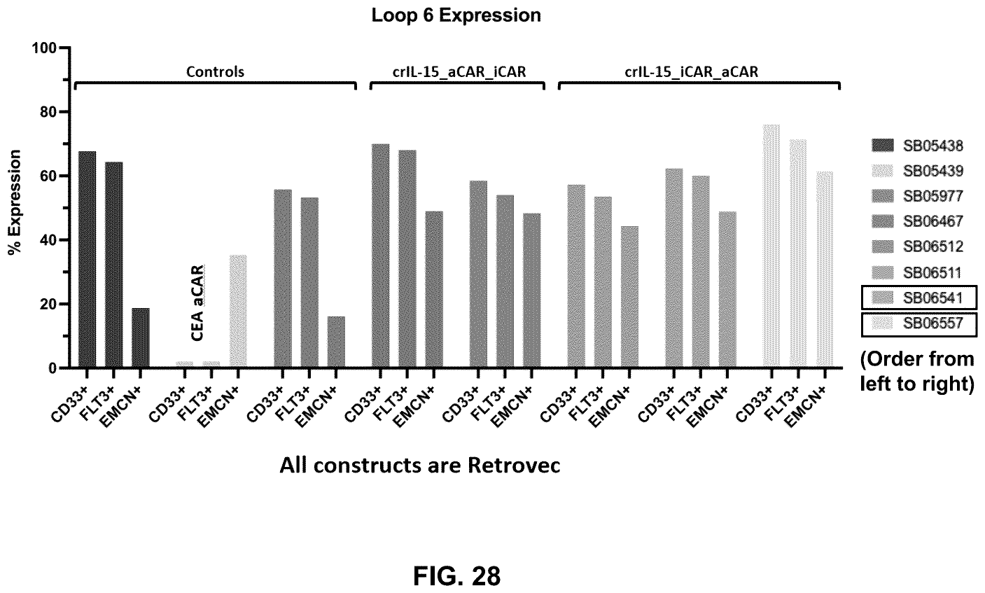

The expression of constructs including either Loop 4 or Loop 6 linkers were assessed for various constructs by flow cytometry. A summary of CD33/EMCN double positive cells is shown in FIG. 26, with the results demonstrating double positive populations of CD33+ and EMCN+ CAR NK cells ranged from 40-60%. A summary of expression for each of the CD33, FLT3, and EMCN CARs for constructs with Loop 4 linkers is shown in FIG. 27, demonstrating expression levels of CD33 and FLT3 populations were nearly identical and EMCN expression generally detectable. A summary of expression for each of the CD33, FLT3, and EMCN CARS for constructs with Loop 6 linkers is shown in FIG. 28, also demonstrating expression levels of CD33 and FLT3 populations were nearly identical and EMCN expression generally detectable.

### Loop 4/Loop 6 and Tagless Cytotoxicity Assessment

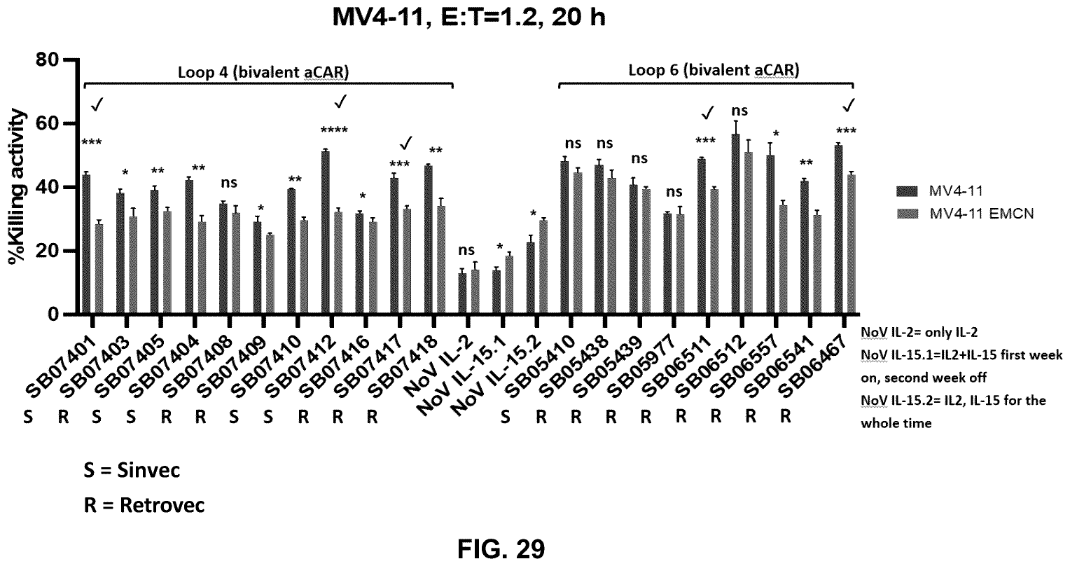

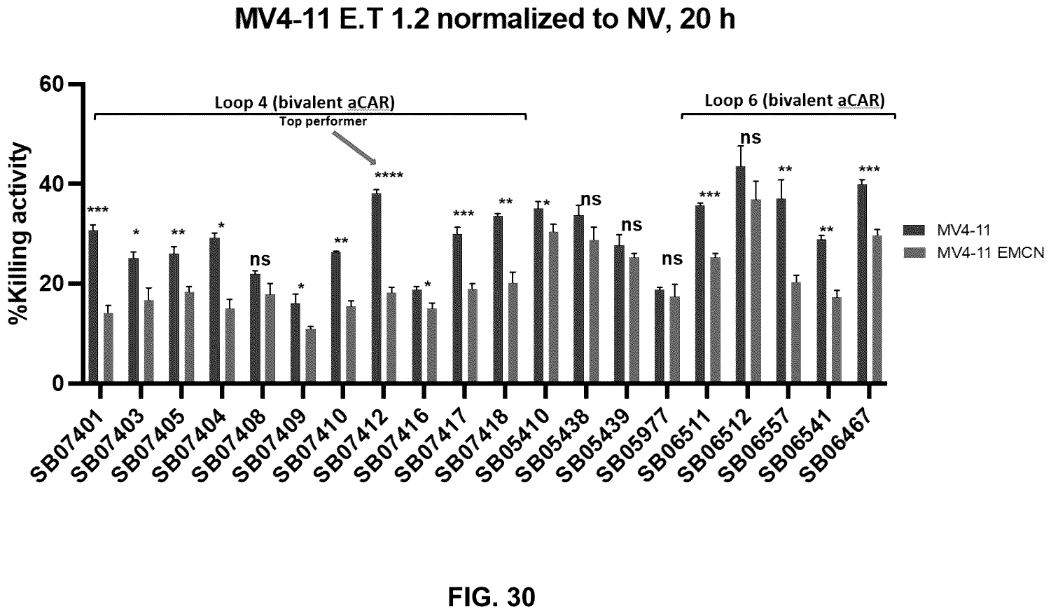

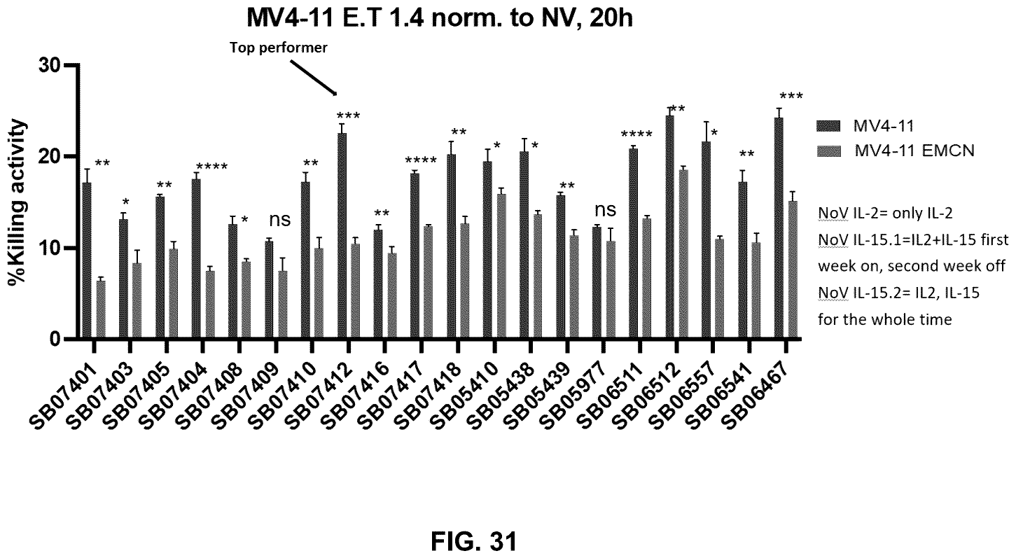

Cytotoxicity for constructs including either Loop 4 or Loop 6 linkers, as well as tagless versions of the EMCN iCAR and the FLT3/CD33 bivalent aCAR, were assessed for various constructs. A summary of killing activity for effector:target ratio of 1:2 is shown in FIG. 29 and normalized to no virus (NV) in FIG. 30. A summary of killing activity for effector:target ratio of 1:4 is shown in FIG. 31. The results demonstrated significant CAR-specific killing and significant EMCN-dependent protection of MV4-11 target cells. While there was lower overall killing for 1:4 E:T ratio, there was still significant killing activity and EMCN iCAR mediated protection.

### Example 4—In Vitro Characterization of SEM Target Cells

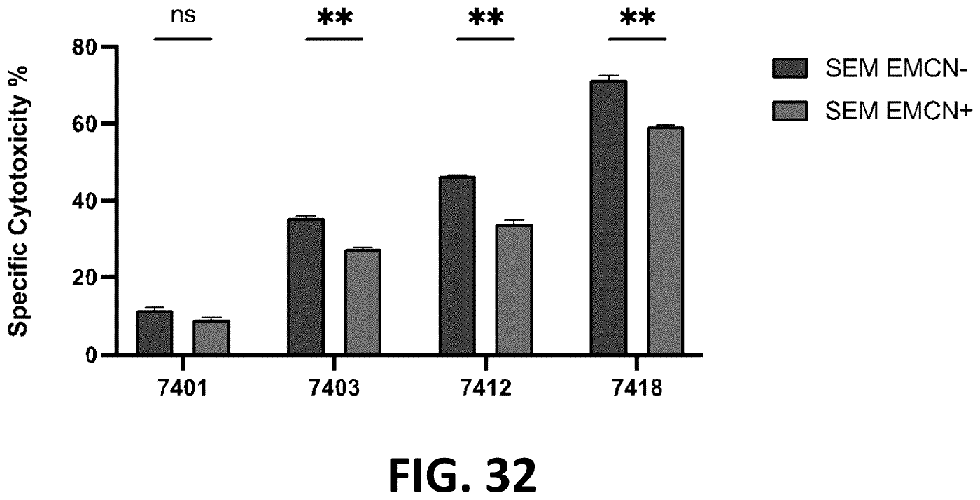

A cell cytotoxicity assay was performed using SEM cells to assess the logic gating of an aCAR/iCAR system in NK cells expressing the aCAR/iCAR. In FIG. 32, NK cells transduced with SB07401, SB07403, SB07412, and SB07418 were co-cultured with SEM cells expressing EMCN (“EMCN+”) or not expressing EMCN (“EMCN−”) for 20 hours. FIG. 32 shows an assessment of specific cytotoxicity and iCAR protection on Day 13 post-transduction (1:2 effector-to-target ration). Specific cytotoxicity was calculated by normalization through subtracting the background cytotoxicity of untransduced NK cells. The study demonstrated that SB07403, SB07412, and SB07418 provided specific aCAR-mediated cytotoxicity of EMCN− SEM cells, as well as protection of EMCN+ SEM cells through the iCAR.

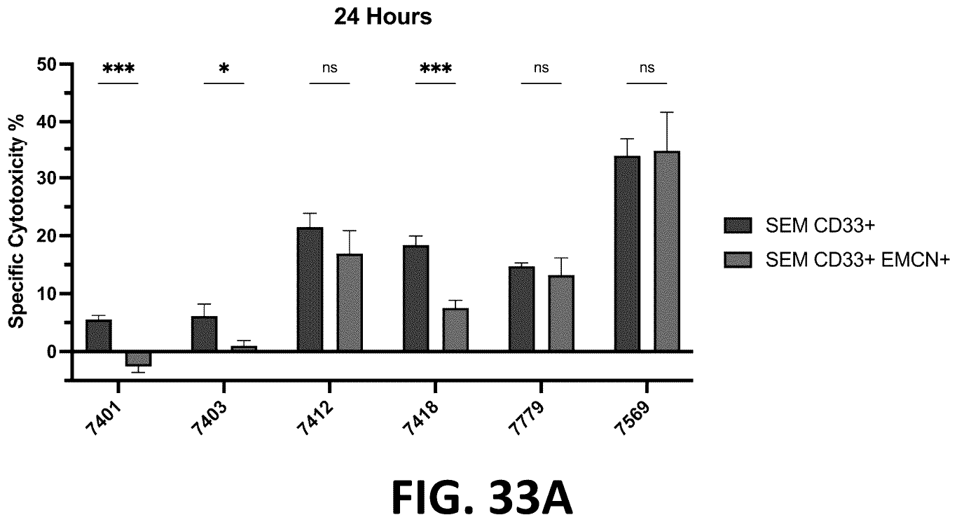

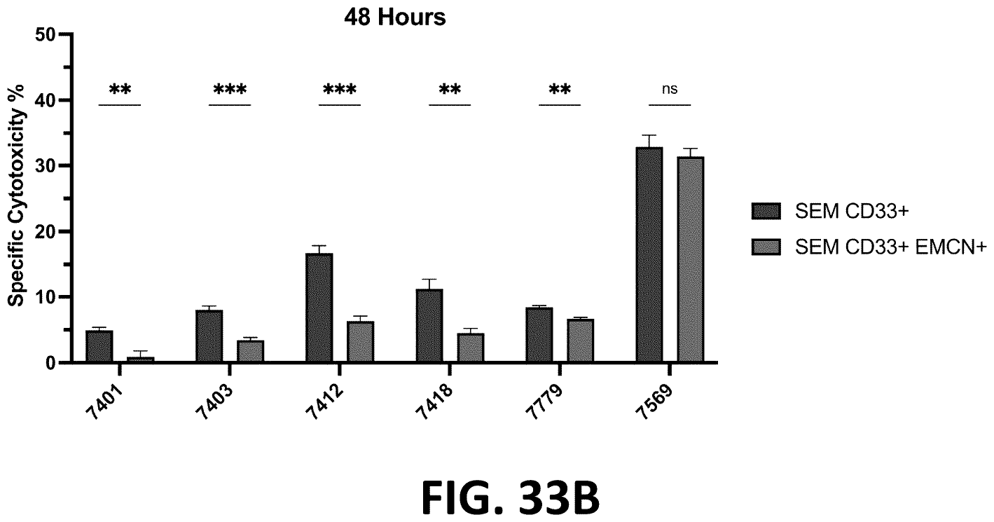

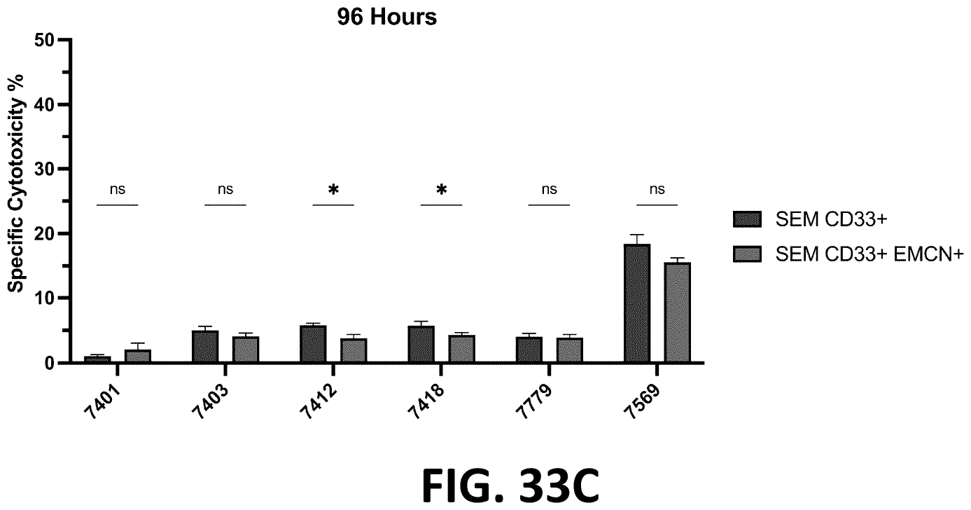

A further cytotoxicity assay was performed for a serial killing assay where multiple rounds of co-culturing with CD33+ SEM cells expressing EMCN (SEM CD33+ EMCN+) or not expressing EMCN (SEM CD33+) were performed. Serial killings were performed at 24 hours, 48 hours, and 96 hours. FIG. 33A-FIG. 33C shows specific killing and protections after 2 challenges. SB07412 and SB07418 demonstrated significant CAR-specific killing and iCAR protection after two re-challenges.

### Example 5—In Vivo

### SEM In Vivo Study

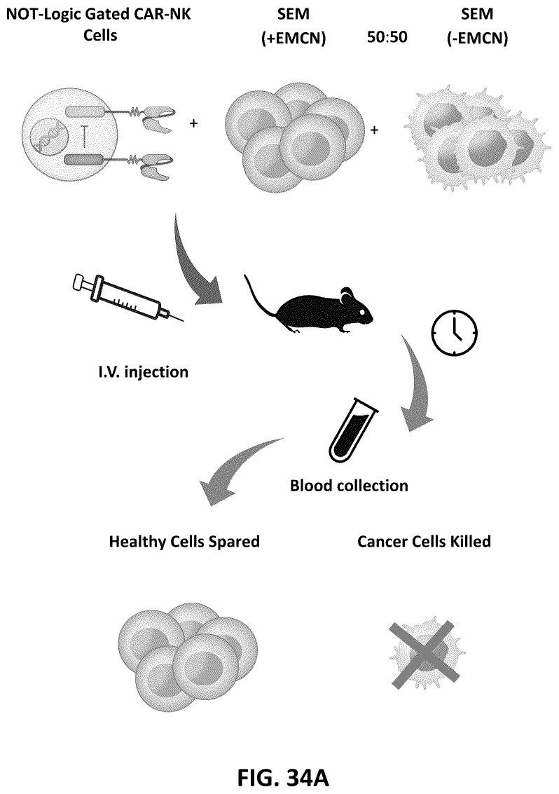

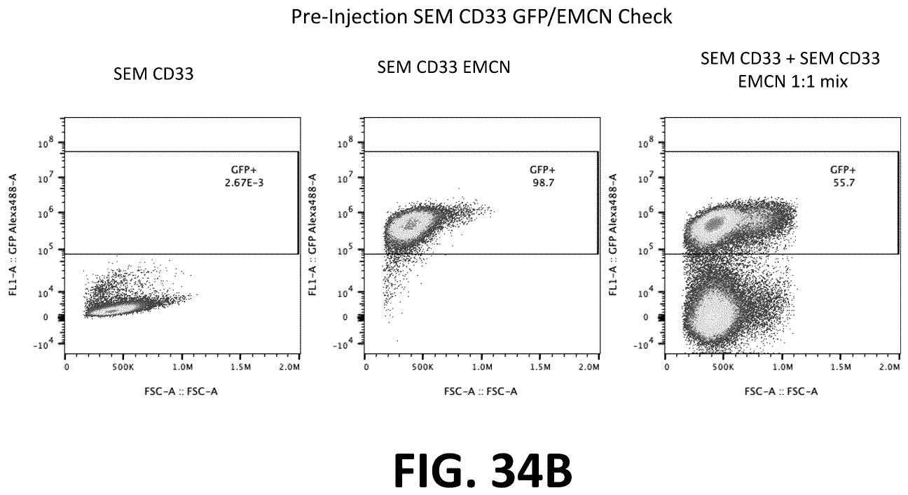

The aCAR/iCAR constructs were tested in an in vivo mouse model. JAX IL15 NSG mice were infection with a mixture of tumor cells and NK cells (1:1 ratio) at day 0. IL-2 was injected twice per week. Table G shows the constructs used for this study. FIG. 34A shows a general schematic of the experimnet. FIG. 34B shows the expression of the SEM population comprising a 1:1 mixture of SEM cells expressing EMCN (CD33+/EMCN+) and SEM cells not expressing EMCN (CD33+/EMCN−).

The aCAR/iCAR constructs were tested in an in vivo mouse model. JAX IL15 NSG mice were injected with a mixture of tumor cells and NK cells (1:1 ratio) at day 0. IL-2 was injected twice per week. Table G shows the constructs used for this study. FIG. 34A shows a general schematic of the experimnet. FIG. 34B shows the expression of the SEM population comprising a 1:1 mixture of SEM cells expressing EMCN (CD33+/EMCN+) and SEM cells not expressing EMCN (CD33+/EMCN−).

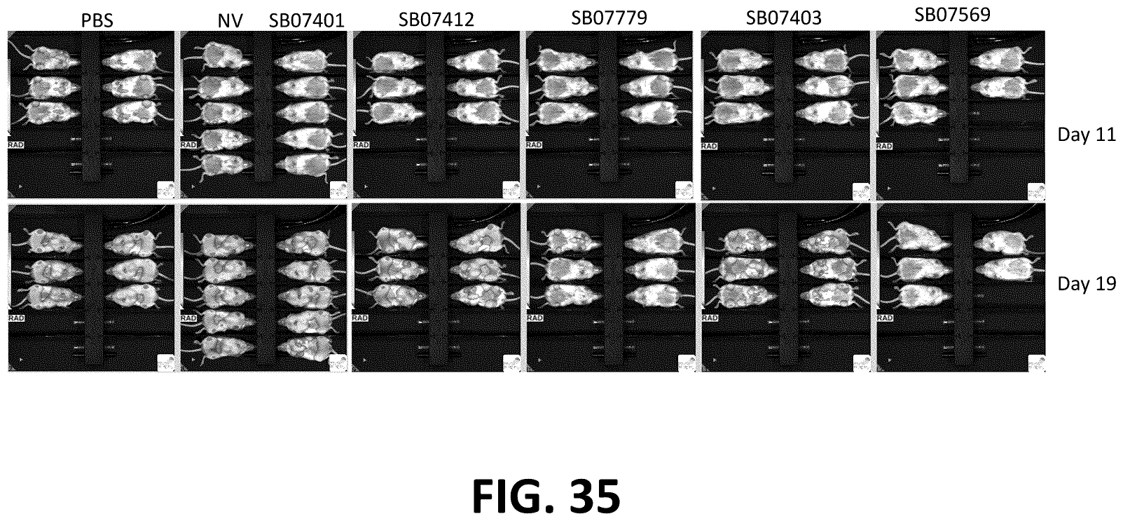

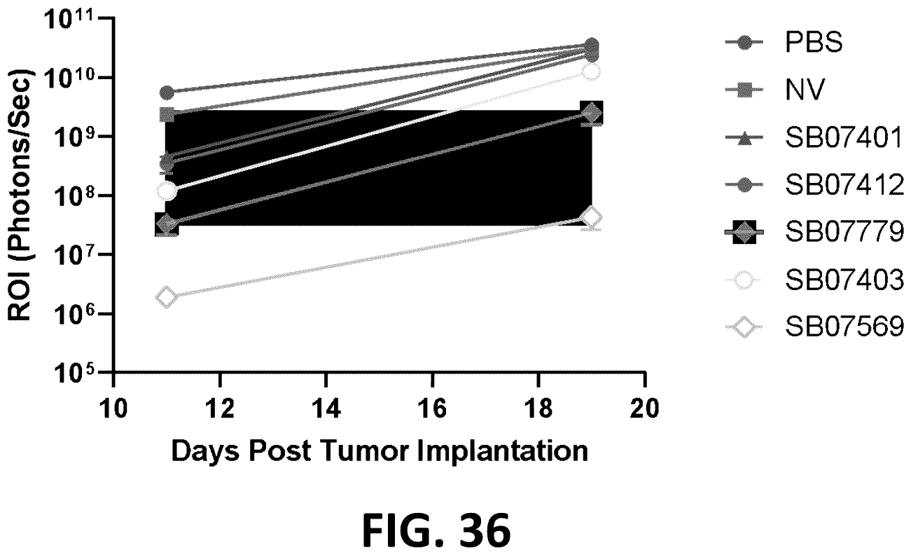

Following injection of the SEM cell population, the animals were visualized by bioluminesence imaging (BLI) on days 11 and 19 (days after SEM injection). In addition to BLI, blood is collected from mice and SEM cells quantified. FIG. 35 shows mice treated wth PBS or NK cells transduced with no virus (NV), SB07401, SB07779, SB07403, or SB07569. BLI measurements were performed at specified time points after SEM cell injection. On day 19, mice injected with NK cells transduced with SB07779 and SB07569 exhibited reduced BLI as compared to mice injected with untransduced NK cells (NV). FIG. 36 shows BLI quantification of the region of interested from time points, day 11 and day 19. Table H summarizies the observations of the study as noted in Table G. SB07779, SB07569 and SB07403 groups desmonstrated lower BLI quantification at day 19 as compared to NV Group.

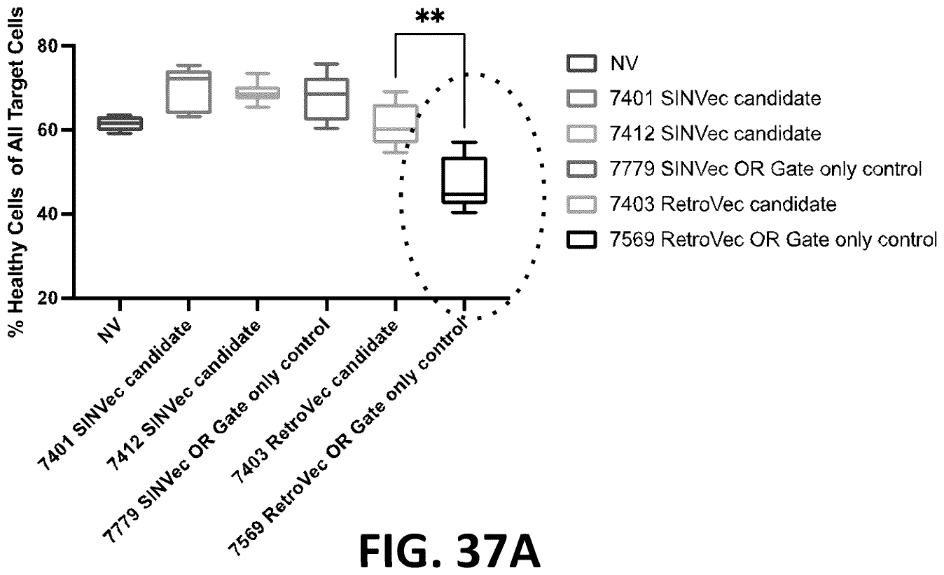

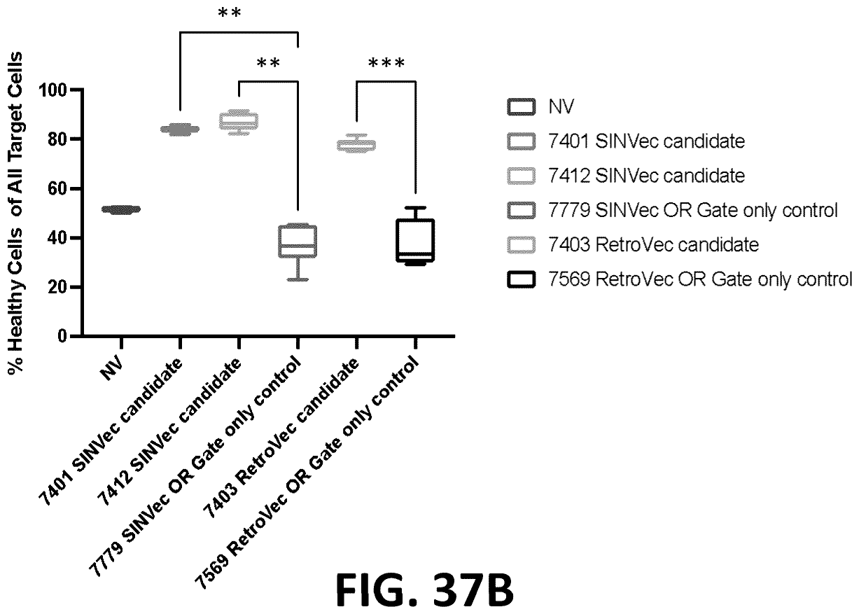

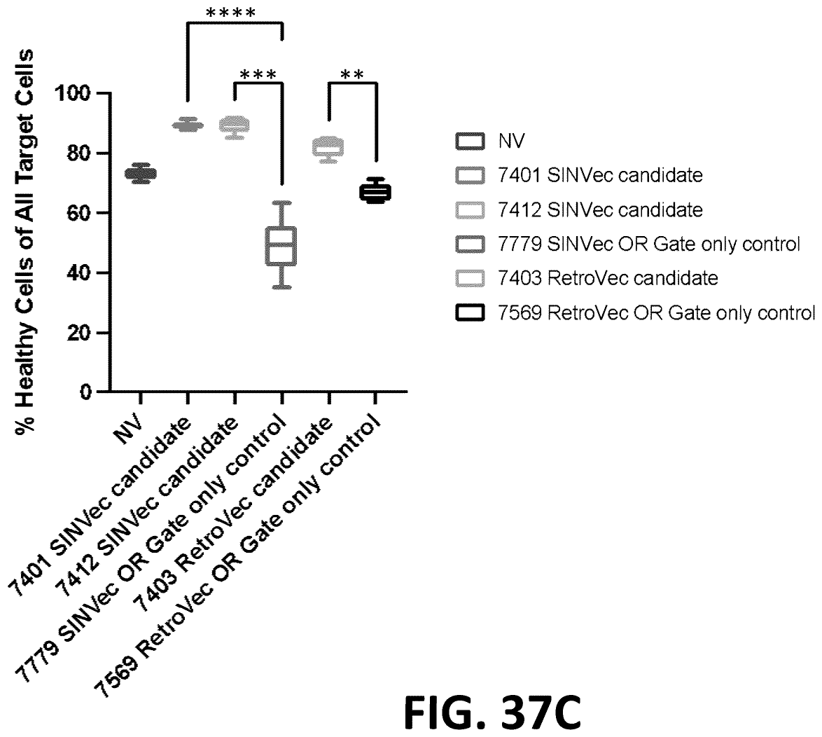

To assess the effects of protection of cells expressing the safety antigen (EMCN), peripheral blood was drawn from animals and quantified. Expression of the safety antigen was used as a model for healthy cells, thus EMCN+ SEM cells should be protected from cell killing in the presence of NK cells expressing the aCAR and the EMCN-specific iCAR. FIG. 37A shows quantification of proportion of SEM cells in the peripheral blood after injection of the NK cells transduced with the respective constructs. Mice were injected with a 1:1 ratio of EMCN(+) to EMCN(−) SEM cells, thus an increase in the percentage of EMCN+ cells would be indicative of iCAR expression when compared to the treatment group consisting of NK cells expressing the aCAR alone (OR Gate only control). On day 13, peripheral blood was drawn and the proportion of EMCN+ cells were quantified using flow cytometry. Analysis of the peripheral blood found that NK cells transduced with SB07403 (aCAR/iCAR) signifcantly protected EMCN+ SEM cells as compared to the NK cells transduced with the OR gate control SB06569. FIG. 37B shows the results of peripheral blood quantification of SEM in mice at day-21. At this timepoint, NK cells trasnsduced with 7401 and 7412 showed increased protection of EMCN+ cells as compared to the NK cells transduced with 7779 OR gate only control, resulting in EMCN+ cells as the more dominant population as compared to EMCN− SEM cells. The significant protective effect of the iCAR (as observed at day 13) persists for the the Retrovec candidate when compared to its respecitve OR gate only control 7569. On day-27, the peripheral blood was again analyzed to determine whether the iCAR mediated protection persisted at longer time points. It was found that the proporation of EMCN positive cells remained high at day 27 and 7401, 7412, and 7403 showed significant protection as compared to their respecitive OR gate control constructs (FIG. 37C).

TABLE G
Experimental Groups used in SEM in vivo study

| Group | Vector |
|---|---|
| PBS | N/A |
| NV | N/A |
| SB07401 | Bivalent aCAR + EMCN iCAR (sinvec) |
| SB07412 | Bivalent aCAR + EMCN iCAR (sinvec) |
| SB07779 | Bivalent aCAR Her2 KLRG1 iCAR (sinvec) |
| SB07403 | Bivalent aCAR + EMCN iCAR (retrovec) |
| SB07418 | Bivalent aCAR + EMCN iCAR (retrovec) |
| SB07569 | Bivalent aCAR + Her2 KLRG1 iCAR |
| | (retrovec) |

TABLE H
Structure and Brief Summary of Study

| | | | | Observation | |
|---|---|---|---|---|---|
| | | Treatment | Mouse | (2-6 hr post | |
| Group | Tumor cells | NK Cells | # | treatment) | Summary |
| 1 | Nalm6-FLT3 (0.25e6) + | PBS | 4 | | |
| 2 | Nalm6-EMCN-FLT3 | NK NV | 4 | | |
| 3 | (0.25e6) 1:1 mix | NK | 4 | | |
| | | SB07401 | | | |
| 4 | | NK | 4 | | |
| | | SB07412 | | | |
| 5 | | NK | 4 | Low body | Recovered |
| | | SB07779 | | temperature, less | |
| | | | | active | |
| 6 | | NK | 4 | Low body | Recovered |
| | | SB07403 | | temperature, less | |
| | | | | active | |
| 7 | | NK | 4 | Low body | 2 out of 4 dead |
| | | SB07418 | | temperature, less | |
| | | | | active | |
| 8 | | NK | 4 | Low body | 3 out of 4 dead |
| | | SB07569 | | temperature, less | |
| | | | | active | |
| 9 | SEM-CD33 (1.5e6) + SEM- | PBS | 6 | | |
| 10 | EMCN-CD33 (1.5e6) 1:1 | NK NV | 5 | | |
| 11 | mix | NK | 5 | | |
| | | SB07401 | | | |
| 12 | | NK | 6 | Low body | Recovered |
| | | SB07412 | | temperature, less | |
| | | | | active | |
| 13 | | NK | 6 | Low body | Recovered |
| | | SB07779 | | temperature, less | |
| | | | | active | |
| 14 | | NK | 6 | | |
| | | SB07403 | | | |
| 15 | | NK | 6 | Low body | 5 out of 6 dead. 1 |
| | | SB07418 | | temperature, less | euthanized |
| | | | | active | |
| 16 | | NK | 6 | Low body | 1 out of 6 dead |
| | | SB07569 | | temperature, less | |
| | | | | active | |

### MV4-11 In Vivo Study

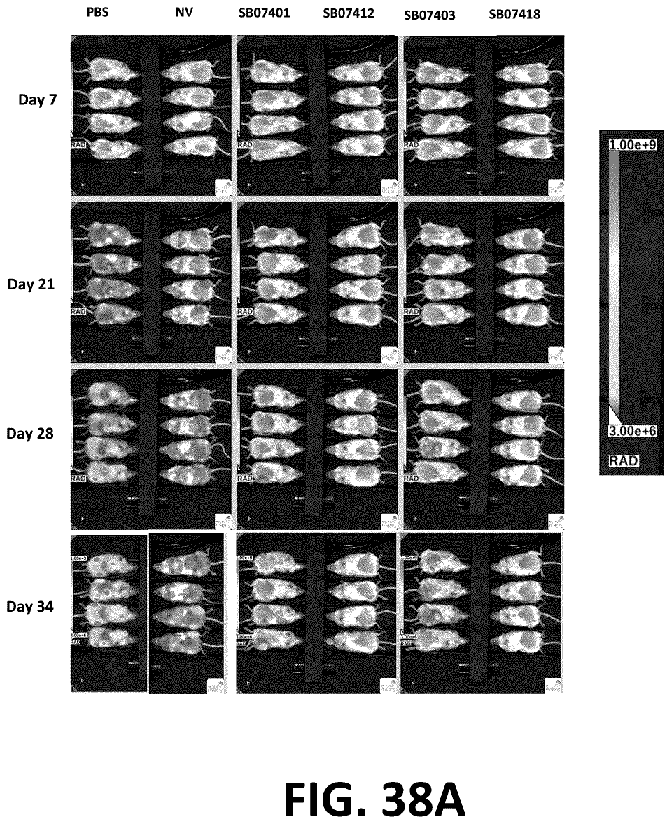

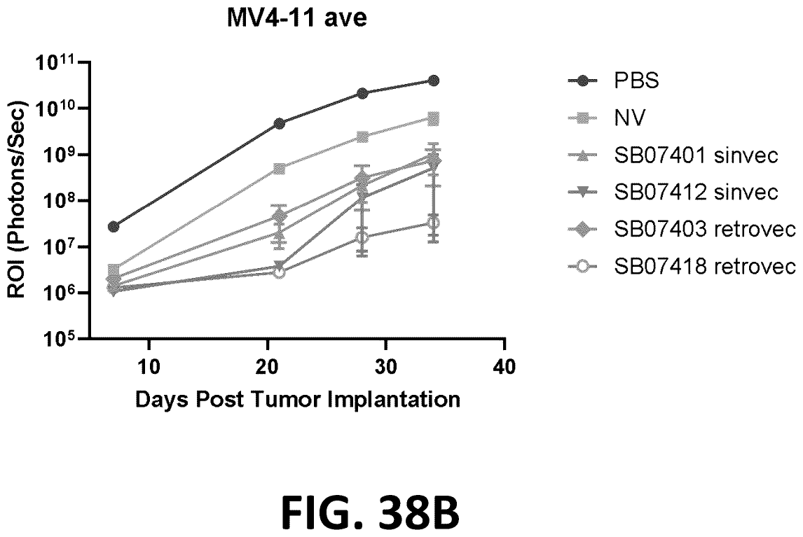

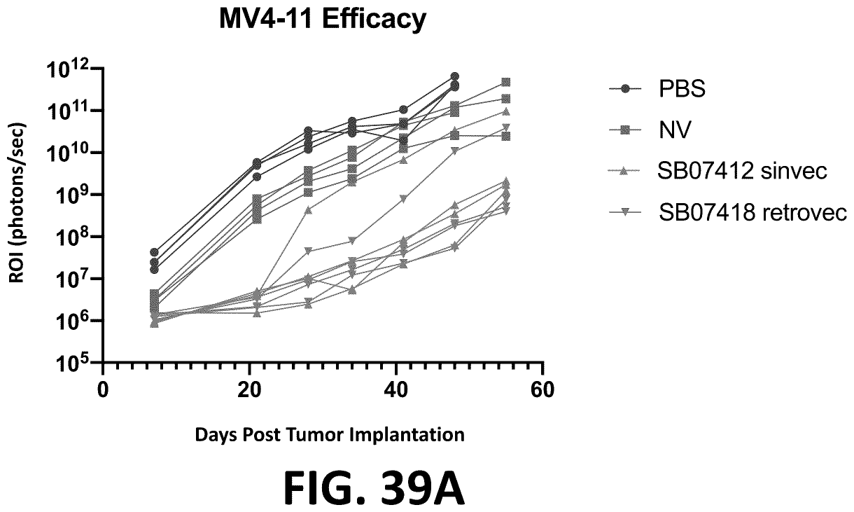

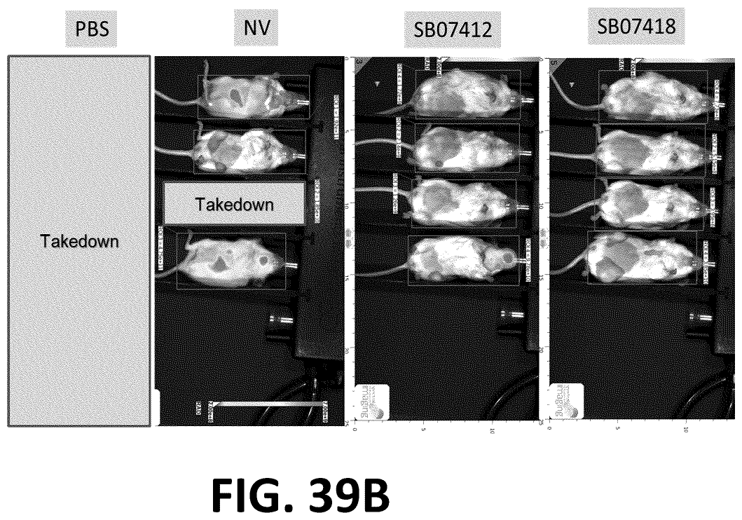

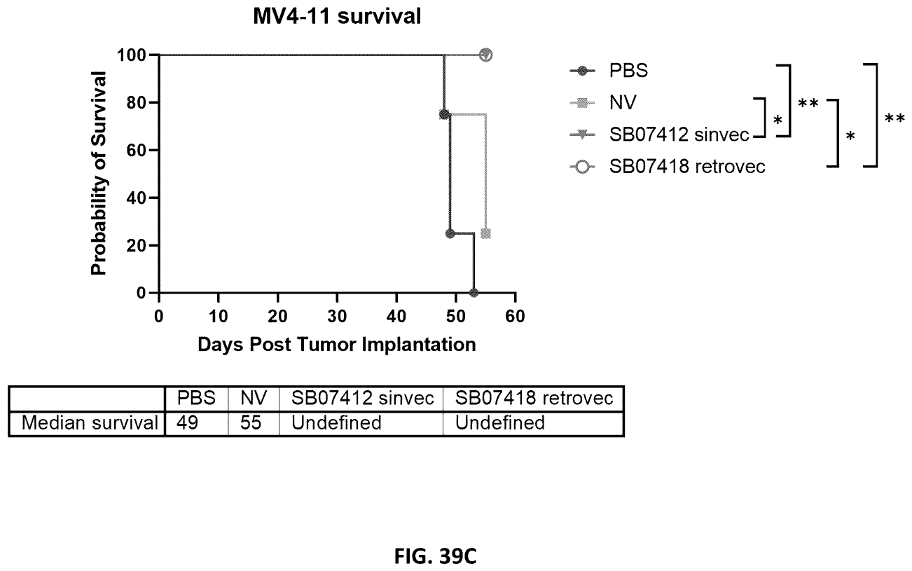

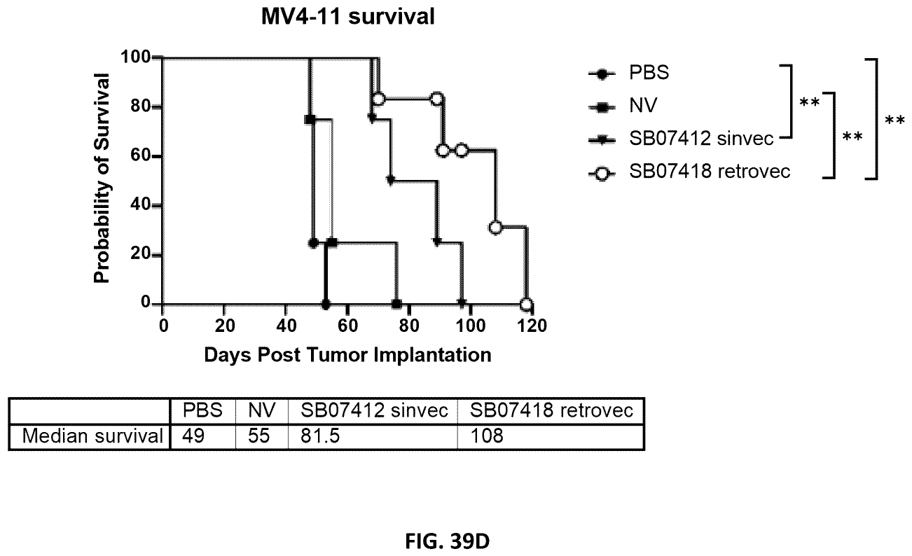

An in vivo study was conducted to assess the functionality of NK cells expressing the aCAR/iCAR, by using MV4-11 target cells in a mouse model. Table I summarizes the experimental groups used in this study and Table J summarizes the results of the study. 50 μL of MV4-11 cells and 150 μL of NK cells were injected into the JAX IL15 NSG mouse on day 0. IL2 was also administered to the animal two times per week. On day 21, IL2 administration was ended. BLI measurements were performed on days 7, 21, 28, and 34. FIG. 38A-B shows representative images and quantification, respectively, of the BLI measurements at the indicated time points. Injection of NK cells transduced with constructs SB7401, SB7412, SB07403, and SB07418 showed reduced tumor growth, as quantified through BLI as compared to the PBS or untransduced NK cell controls. This observation persisted for SB07412 and SB04128 when the study was continued to ˜55 days (FIG. 39A). FIG. 39B shows a representative BLI image of animals injected with PBS, untransduced NK cells, and NK cells transduced with SB07412 and SB07418 at day 55. The image shows that mice receiving NK cells trasnduced with constructs SB07412 and SB07418 exhibited decreased signal at day 55, indicating killing of the injected MV4-11 cells compared to the PBS and transduced NK cell controls. In FIG. 39C, median survival for mice receiving PBS or untransduced NK cells were calculated to be 49 and 55 days respectively, whereas the mice receiving the transduced NK cells (SB07412 and SB07418) have undefined median survival. The differences in the median survival of mice treated with NK cells transduced with SB07412 or SB07418 were found to be significant when compared to mice treated with PBS or untransduced NK cells, showing that the constructs encoded by SB07412 or SB07418 increase survival in an MV4-11 tumor model. In FIG. 39D, the same study of FIG. 39C has been carried out until day 120, in which the median survival (days) was found for mice injected with PBS (49 days), non-engineered NK cells (NV) (55 days), engineered NK cells transduced with SB07412 Sinvec (81.5 days) or SB07418 Retrovec (108 days).

TABLE I

| Group | Vector |
|---|---|
| PBS | N/A |
| NV | N/A |
| SB07401 | Bivalent aCAR + EMCN iCAR (Sinvec) |
| SB07412 | Bivalent aCAR + EMCN iCAR (Sinvec) |
| SB07403 | Bivalent aCAR + EMCN iCAR (Retrovec) |
| SB07418 | Bivalent aCAR + EMCN iCAR (Retrovec) |

Table J shows the quantification of the NK cells after transduction with the respective viruses having the specific construct.

TABLE J

| | Expression | Expression | |
|---|---|---|---|
| | CD33+ | EMCN+ | |
| SB# | % | % | Copy# |
| SB07401(Sin Vec) | 79.3 | 58.8 | 38.1 |
| SB07412(Sin Vec) | 86.3 | 65.8 | 47.7 |
| SB07403 (Retro Vec) | 59.70 | 71.90 | 27.6 |
| SB07418 (Retro Vec) | 80.1 | 58.4 | 25.7 |

### Example 6: Validation of Other iCAR Architectures

In this study, additional ICDs were tested for use in an EMCN iCAR. Table 9 provides the sequences of the full-length anti-EMCN iCARs assessed, which include the various ICDs indicated with all iCARs including the same anti-EMCN binder sequence: iCARs with the various ICDs were tested in a cytotoxicity assay as previously described in the present disclosure.

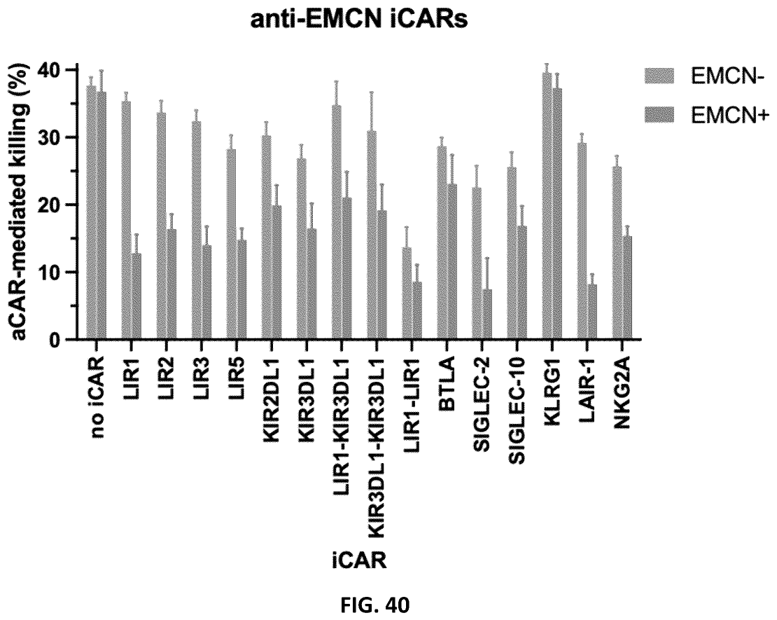

Cells were engineered to express an aCAR and an iCAR with different ICDs to determine the inhibitory effect of various iCAR architectures on the activity of an aCAR. FLT3 NOT EMCN CAR-NK cells expressing iCARs having different ICDs (primary NK cells engineered to co-express the same FLT3 aCAR with different EMCN iCARs) were used in an in vitro cytotoxicity protection assay screen using target cells that expressed only FLT3 or target cells that expressed both FLT3 and EMCN (FIG. 40). The target cell population expressing FLT3 and EMCN should be protected from aCAR-mediated killing if the tested iCAR is functional and able to inhibit the aCAR activity. Multiple functional iCAR architectures were identified that enabled robust target cell killing in the absence of EMCN expression and provided significant antigen-dependent target cell protection in the presence of EMCN.

TABLE 9
List of exemplary EMCN iCARs having different
intracellular signaling domains

| | SEQ ID | |
|---|---|---|
| ICD | NO: | EMCN iCAR + ICD AA Sequence |
| LIR1 | 489 | MALPVTALLLPLALLLHAARPQVQLKESGPGLVQPSQTLSLTCTVSGFSLSRYD |
| | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDV |
| | | IGILVAVILLLLLLLLLFLILRHRRQGKHWTSTQRKADFQHPAGAVGPEPTDRG |
| | | LQWRSSPAADAQEENLYAAVKHTQPEDGVEMDTRSPHDEDPQAVTYAEVKHSRP |
| | | RREMASPPSPLSGEFLDTKDRQAEEDRQMDTEAAASEAPQDVTYAQLHSLTLRR |
| | | EATEPPPSQEGPSPAVPSIYATLAIH |
| LIR2 | 490 | MALPVTALLLPLALLLHAARPQVQLKESGPGLVQPSQTLSLTCTVSGFSLSRYD |
| | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDV |
| | | IGILVAVVLLLLLLLLLFLILRHRRQGKHWTSTQRKADFQHPAGAVGPEPTDRG |
| | | LQWRSSPAADAQEENLYAAVKDTQPEDGVEMDTRAAASEAPQDVTYAQLHSLTL |
| | | RRKATEPPPSQEREPPAEPSIYATLAIH |
| LIR3 | 491 | MALPVTALLLPLALLLHAARPQVQLKESGPGLVQPSQTLSLTCTVSGFSLSRYD |
| | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDV |
| | | LIGVSVAFVLLLFLLLFLLLRRQRHSKHRTSDQRKTDFQRPAGAAETEPKDRGL |
| | | LRRSSPAADVQEENLYAAVKDTQSEDRVELDSQSPHDEDPQAVTYAPVKHSSPR |
| | | REMASPPSSLSGEFLDTKDRQVEEDRQMDTEAAASEASQDVTYAQLHSLTLRRK |
| | | ATEPPPSQEGEPPAEPSIYATLAIH |
| LIR5 | 492 | MALPVTALLLPLALLLHAARPQVQLKESGPGLVQPSQTLSLTCTVSGFSLSRYD |
| | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDV |
| | | LIGVLVVSILLLSLLLFLLLQHWRQGKHRTLAQRQADFQRPPGAAEPEPKDGGL |
| | | QRRSSPAADVQGENFCAAVKNTQPEDGVEMDTRQSPHDEDPQAVTYAKVKHSRP |
| | | RREMASPPSPLSGEFLDTKDRQAEEDRQMDTEAAASEAPQDVTYAQLHSFTLRQ |
| | | KATEPPPSQEGASPAEPSVYATLAIH |
| KIR2DL1 | 493 | MALPVTALLLPLALLLHAARPQVQLKESGPGLVQPSQTLSLTCTVSGFSLSRYD |
| | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDI |
| | | LIGTSVVIILFILLFFLLHRWCSNKKNAAVMDQESAGNRTANSEDSDEQDPQEV |
| | | TYTQLNHCVFTQRKITRPSQRPKTPPTDIIVYTELPNAESRSKVVSCP |
| KIR3DL1 | 494 | MALPVTALLLPLALLLHAARPQVQLKESGPGLVQPSQTLSLTCTVSGFSLSRYD |
| | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDI |
| | | LIGTSVVIILFILLLFFLLHLWCSNKKNAAVMDQEPAGNRTANSEDSDEQDPEE |
| | | VTYAQLDHCVFTQRKITRPSQRPKTPPTDTILYTELPNAKPRSKVVSCP |
| LIR1- | 495 | MALPVTALLLPLALLLHAARPQVQLKESGPGLVQPSQTLSLTCTVSGFSLSRYD |
| KIR3DL1 | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDV |
| | | IGILVAVILLLLLLLLLFLILRHRRQGKHWTSTQRKADFQHPAGAVGPEPTDRG |
| | | LQWRSSPAADAQEENLYAAVKHTQPEDGVEMDTRSPHDEDPQAVTYAEVKHSRP |
| | | RREMASPPSPLSGEFLDTKDRQAEEDRQMDTEAAASEAPQDVTYAQLHSLTLRR |
| | | EATEPPPSQEGPSPAVPSIYATLAIHHLWCSNKKNAAVMDQEPAGNRTANSEDS |
| | | DEQDPEEVTYAQLDHCVFTQRKITRPSQRPKTPPTDTILYTELPNAKPRSKVVS |
| | | CP |
| KIR3DL1 | 496 | MALPVTALLLPLALLLHAARPQVQLKESGPGLVQPSQTLSLTCTVSGFSLSRYD |
| KIR3DL1 | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDI |
| | | LIGTSVVIILFILLLFFLLHLWCSNKKNAAVMDQEPAGNRTANSEDSDEQDPEE |
| | | VTYAQLDHCVFTQRKITRPSQRPKTPPTDTILYTELPNAKPRSKVVSCP |
| LIR1- | 497 | MALPVTALLLPLALLLHAARPQVQLKESGPGLVQPSQTLSLTCTVSGFSLSRYD |
| LIR1 | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDV |
| | | IGILVAVILLLLLLLLLFLILRHRRQGKHWTSTQRKADFQHPAGAVGPEPTDRG |
| | | LQWRSSPAADAQEENLYAAVKHTQPEDGVEMDTRSPHDEDPQAVTYAEVKHSRP |
| | | RREMASPPSPLSGEFLDTKDRQAEEDRQMDTEAAASEAPQDVTYAQLHSLTLRR |
| | | EATEPPPSQEGPSPAVPSIYATLAIH |
| BTLA | 498 | MALPVTALLLPLALLLHAARPQVQLKESGPGLVQPSQTLSLTCTVSGFSLSRYD |
| | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDL |
| | | LPLGGLPLLITTCFCLFCCLRRHQGKQNELSDTAGREINLVDAHLKSEQTEAST |
| | | RQNSQVLLSETGIYDNDPDLCFRMQEGSEVYSNPCLEENKPGIVYASLNHSVIG |
| | | PNSRLARNVKEAPTEYASICVRS |
| SIGLEC | 499 | MALPVTALLLPLALLLHAARPQVQLKESGPGLVQPSQTLSLTCTVSGFSLSRYD |
| 2 | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDV |
| | | AVGLGSCLAILILAICGLKLQRRWKRTQSQQGLQENSSGQSFFVRNKKVRRAPL |
| | | SEGPHSLGCYNPMMEDGISYTTLRFPEMNIPRTGDAESSEMQRPPPDCDDTVTY |
| | | SALHKRQVGDYENVIPDFPEDEGIHYSELIQFGVGERPQAQENVDYVILKH |
| SIGLEC | 500 | MALPVTALLLPLALLLHAARPQVQLKESGPGLVQPSQTLSLTCTVSGFSLSRYD |
| 10 | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDG |
| | | AFLGIGITALLFLCLALIIMKILPKRRTQTETPRPRFSRHSTILDYINVVPTAG |
| | | PLAQKRNQKATPNSPRTPLPPGAPSPESKKNQKKQYQLPSFPEPKSSTQAPESQ |
| | | ESQEELHYATLNFPGVRPRPEARMPKGTQADYAEVKFQ |
| KLRG1 | 501 | MALPVTALLLPLALLLHAARPQVQLKESGPGLVQPSQTLSLTCTVSGFSLSRYD |
| | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDV |
| | | AIALGLLTAVLLSVLLYQWIMTDSVIYSMLELPTATQAQNDYGPQQKSSSSRPS |
| | | CSCLGSG |
| LAIR1 | 502 | MALPVTALLLPLALLLHAARPQVQLKESGPGLVQPSQTLSLTCTVSGFSLSRYD |
| | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDI |
| | | LIGVSVVFLFCLLLLVLFCLHRQNQIKQGPPRSKDEEQKPQQRPDLAVDVLERT |
| | | ADKATVNGLPEKDRETDTSALAAGSSQEVTYAQLDHWALTQRTARAVSPQSTKP |
| | | MAESITYAAVARH |
| NKG2A | 503 | MALPVTALLLPLALLLHAARPQVQLKESGPGLVQPSQTLSLTCTVSGFSLSRYD |
| | | MHWVRQPPGQGLEWMGVIWGNGNTHYHSALKSRLSISRDTSKSQVFLKMNSLQT |
| | | EDTAIYFCTLRIKDWGPGTMVTVSSGGGGSGGGGSGGGGSDIVMTQTPPSLSVA |
| | | LGQSVSISCKSSQSLVASDENTYLNWLLQSPGRSPKRLIYQVSKLDSGVPDRFS |
| | | GSGSEKDFTLKISRVEAEDLGVYYCLQGIHLPWTFGGGTKLELKGKPIPNPLLG |
| | | LDSTNGAATTTPAPRPPTPAPTIALQPLSLRPEACRPAAGGAVHTRGLDFACDV |
| | | IGILVAVILLLLLLLLLFLIMDNQGVIYSDLNLPPNPKRQQRKPKGNKNSILAT |
| | | EQEITYAELNLQKASQDFQGNDKTYHCKDLPSAPEK |

### Example 7: Characterization of FLT3/CD33 NOT EMCN Expressing NK Cells Derived from Different Donors

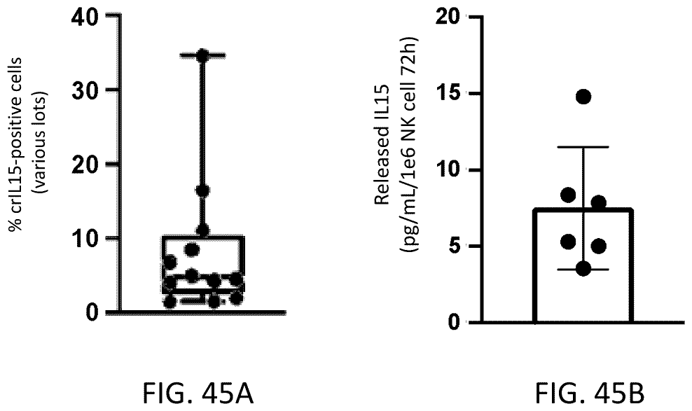

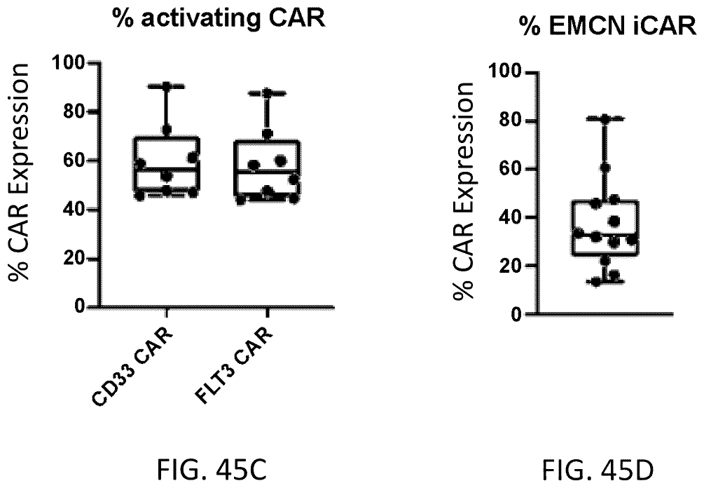

In this study, NK cells obtained from different donors were characterized to assess donor-to-donor variability. These cells were non-engineered or engineered with the FLT3/CD33 NOT EMCN Circuit (SB07412). NK cells derived from different donors were characterized for their ability to express different components within SB07412 (FLT3/CD33 bivalent aCAR, EMCN iCAR, and crIL15). FIGS. 45A-45D show expression of crIL15-positive NK cells (FIG. 45A), secreted crIL15 (FIG. 45B), the CD33/FLT3 bivalent aCAR (FIG. 45C), and the EMCN iCAR (FIG. 45D).

### NK Cell Cytotoxicity

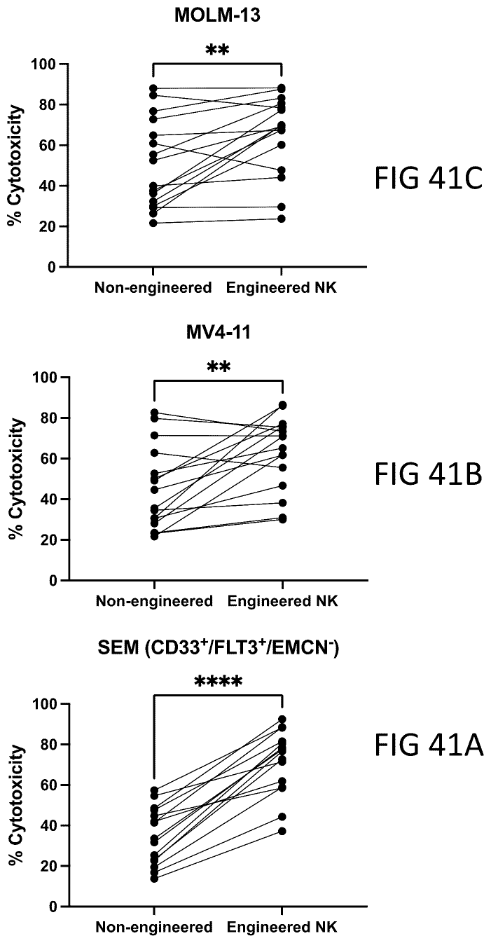

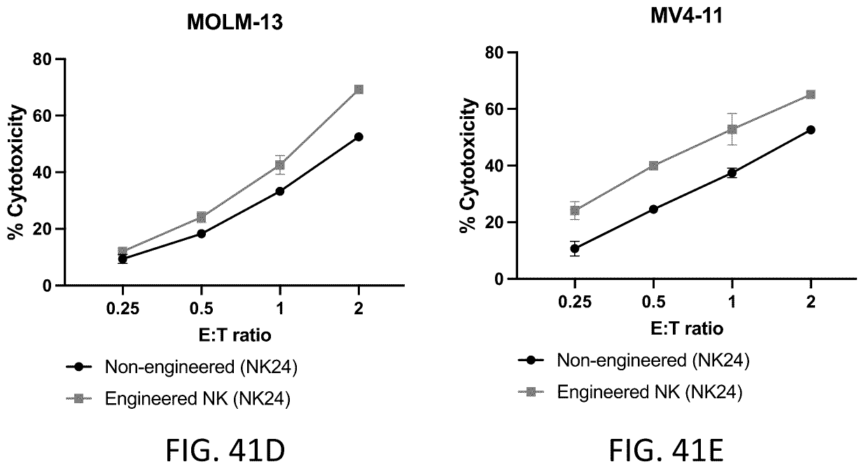

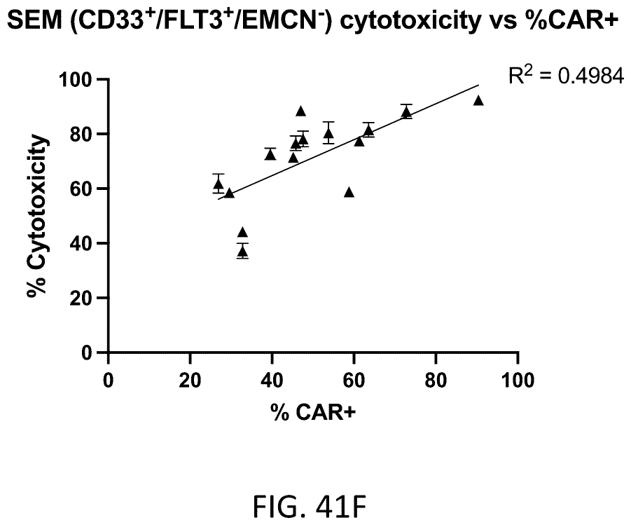

In FIG. 41A-41F, non-engineered or engineered NK cells were tested for cell-mediated cytotoxicity against AML cell lines line MOLM-13 or MV4-11, and SEM (FLT3+/CD33+/EMCN−). A total of 16 lots of NK cells, produced from 11 different NK donors, were used in these assays. When cytotoxicity data was compared across all tested lots of NK cells, the average cytotoxicity induced by the transduced cells was significantly higher than that observed from non-engineered NK cells in all cell lines (FIGS. 41A-41C). For both the engineered NK cells and non-engineered NK cells, the amount of cytotoxicity observed was dependent on the E:T ratio used in the experiment (FIG. 41D and FIG. 41E), indicating that the cytotoxic effect correlates with E:T ratio. In the case of cell-mediated cytotoxicity against SEM (CD33+/FLT3+/EMCN−) target cells, the observed cytotoxicity was further observed to correlate with activating CAR expression on engineered NK cells (FIG. 41F).

Cytotoxicity of engineered NK cells and non-engineered NK cells from 16 different lots was tested against MOLM-13, MV4-11, and SEM-CD33 cell lines. NK cytotoxicity against MOLM-13 (FIG. 41A), MV4-11 (FIG. 41B), and SEM-CD33 (FIG. 41C) cells across multiple NK lots (each line represents a different NK lot), at an E:T ratio of 2:1 (FIGS. 41A-41C). Each point represents the mean cytotoxicity value from one experiment performed in triplicate, and each line connects a paired set of data points (non-engineered and engineered NK cells) from the same lot of cells tested. Statistics performed using paired t-tests; **p<0.01, ****p<0.0001. FIGS. 41D-41E demonstrate NK cytotoxicity against MOLM-13 (FIG. 41D) and MV4-11 (FIG. 41E) cells at multiple E:T ratios. Data is shown from a single NK cell donor. Error bars represent sample standard deviation. FIG. 41F shows cytotoxicity of engineered NK cells and non-engineered NK cells from multiple experiments plotted against CD33 CAR expression. Error bars represent standard deviation across samples in triplicate, and data was fit using linear regression.

### Cytokine Profile of Engineered NK Cells

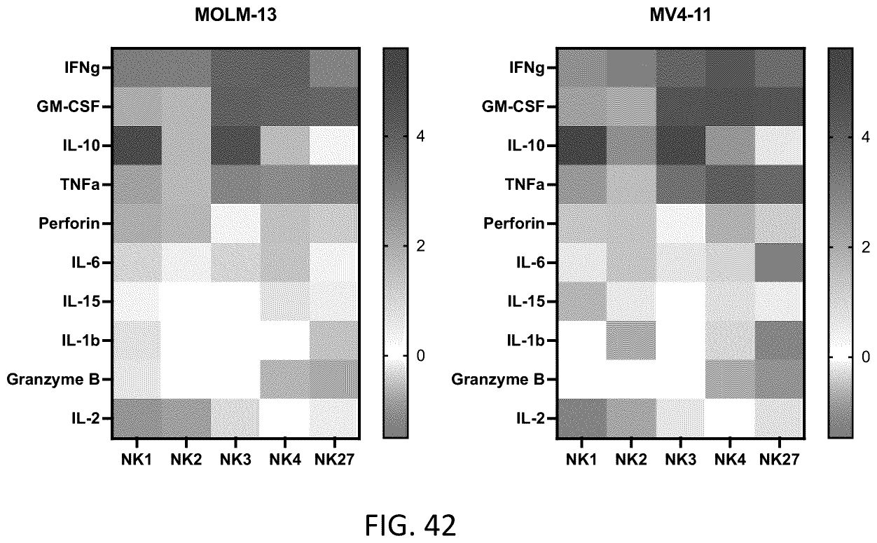

In FIG. 42, the cytokine profiles were obtained from the cell supernatant of non-engineered or engineered NK cells following the first round of serial killing of MOLM-13 or MV4-11. In FIG. 42, a heat map was generated by analysis of the specified cytokine profiles, comparing the cytokine levels between the engineered and unengineered NK cells for the respective cell lines that the NK cells were co-cultutred with. Fold expression was calculated using the values generated using the formula: fold expression=log₂ ((engineered NK cell cytokine level)/(nonengineered NK cell cytokined level)).

Higher levels of cytokines in the supernatant were measured in the engineered NK cells compared to non-engineered NK cells from the same donor (FIG. 42). Values were calculated by determining the fold change of cytokines measured in engineered NK cell samples compared to non-engineered NK samples from the same lot. The cytokines with increased production from co-culturing with engineered NK cells were IFNγ, GM-CSF, IL10, TNFα, and perforin which all mediate inflammatory or cytotoxic functions in NK cells. Cytokines were not detectable when NK cells were not added to the AML cell line culture, suggesting that cytokine production was due to presence of NK cells rather than expressed inherently by the target cell lines.

Cytokines assayed from supernatants of the engineered NK cells and non-engineered NK cells from 5 different lots after co-culture with MOLM-13 and MV4-11 AML target cells at an E:T ratio of 2:1.

### Engineered NK Cell Cytotoxicity Against Primary AML Samples

Primary AML patient samples from different subtypes were purchased from Discovery Life Sciences. Cryopreserved samples were thawed and cultured overnight for assays. The day after, thawed and rested NK cells (engineered NK cells and donor-matched non-engineered NK cell controls) were co-cultured with primary AML cells at 2:1 E:T ratio overnight. Viability of AML target cells was determined via flow cytometry using a viability dye (Sytox Red). To distinguish between AML blasts and LSCs, CD45low target cells were further profiled using CD34 and CD38. AML blasts=CD56-CD45lowSSClow; LSC=CD56-CD45lowCD34highCD38low.

Engineered cells were further tested for cytotoxicity against primary tumor-derived AML samples. AML patient samples from M0, M1, M4 and M5 subtypes were co-cultured with engineered NK cells or donor-matched non-engineered NK cells. Cytotoxicity was determined after an overnight co-culture in at 37° C./5% CO2 incubator via flow cytometry gating on AML blasts and leukemic stem cells (LSCs).

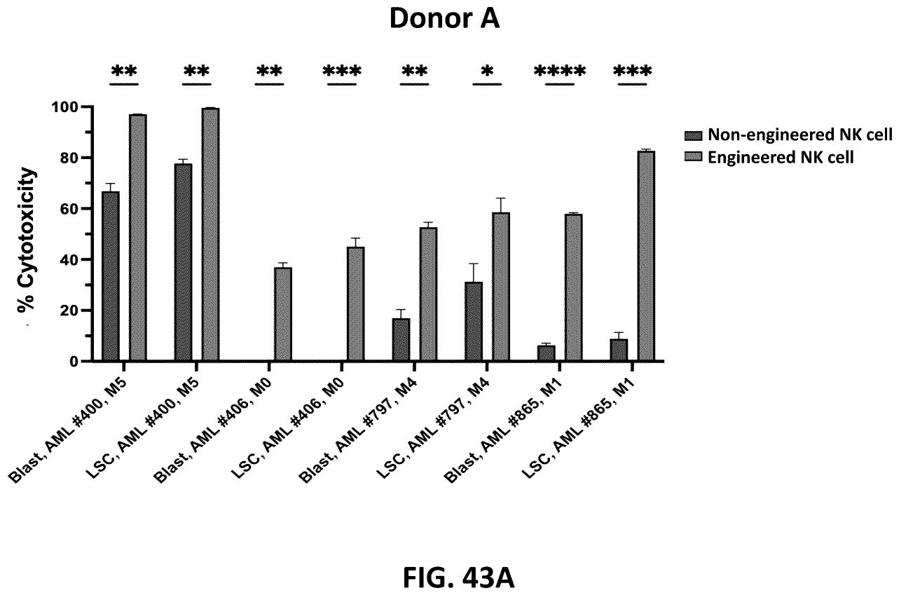

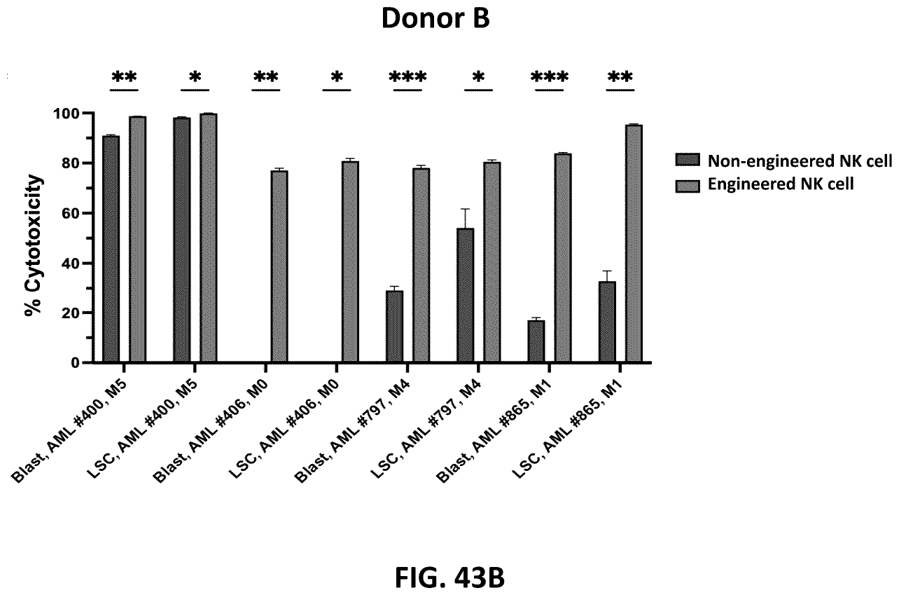

In both lots of engineered NK cells tested, engineered NK cells demonstrated high target cell killing of primary tumor-derived AML samples. Cytotoxicity was demonstrated against each primary AML sample tested, including against both AML blasts and LSC cell types (FIGS. 43A-43B). Notably, engineered NK cells demonstrated significantly greater cytotoxicity than non-engineered NK cells against primary AML blasts and LSCs in all samples tested.

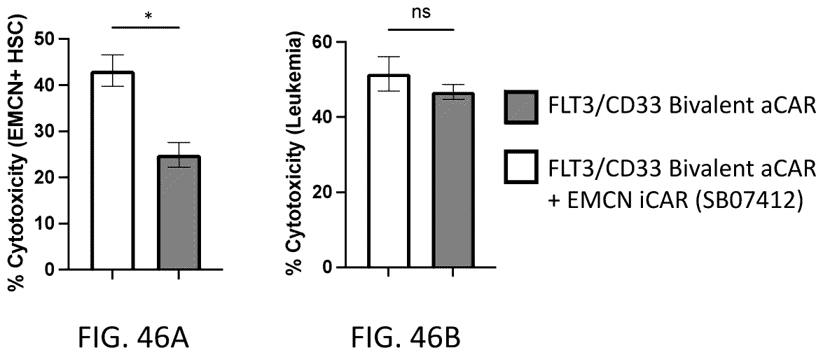

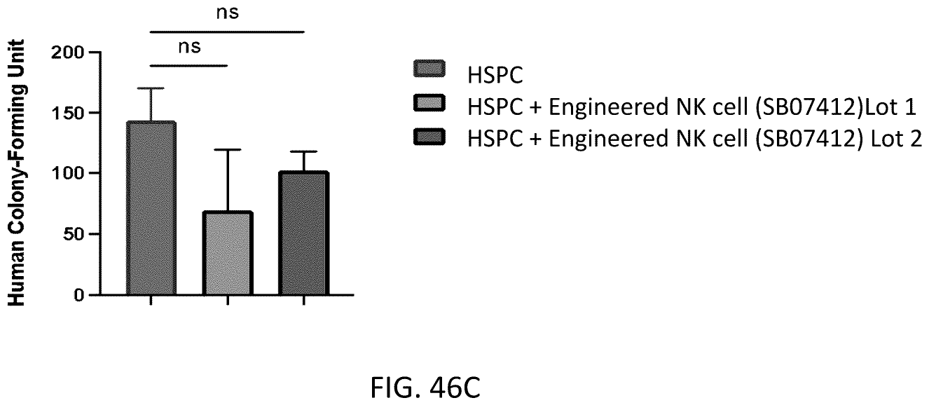

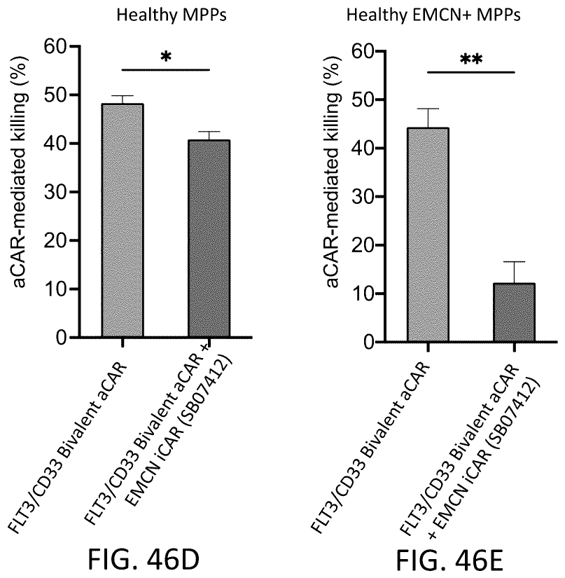

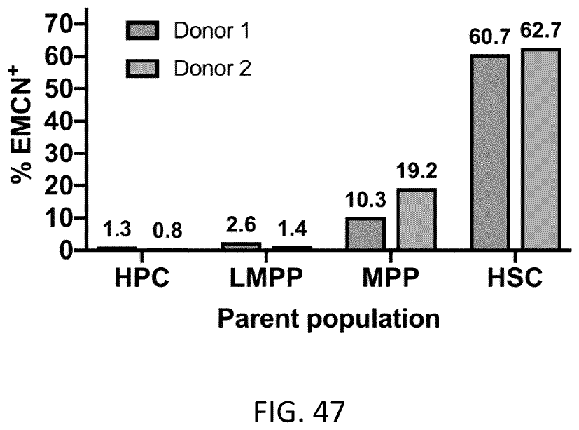

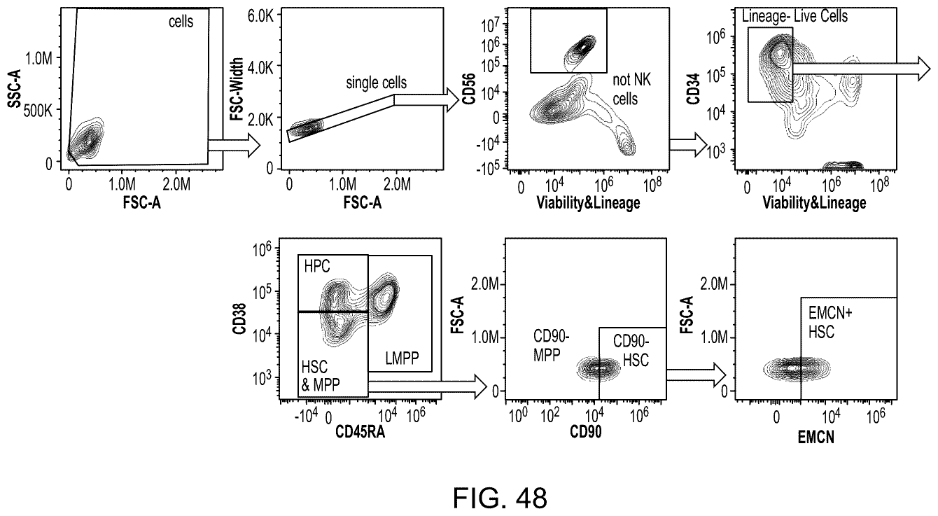

iCAR Mediated Protection of Human Stem Cells Using the engineered NK cells (SB07412), the previously disclosed examples describe protection of cells that express the protective antigen EMCN as well as the target antigens, CD33 and/or FLT3. EMCN was chosen as a protective antigen for healthy hematopoietic stem cells (HSCs) and other progenitor cells since it is specifically expressed in these healthy cell subsets (See FIG. 47 for representative EMCN expression in HSCs, multipotent progenitors (MPPs), lympho-myeloid primed progenitor (LMPP), and hematopoietic progenitor cell (HPC)). In this study, protection of human hematopoietic stem cells (HSCs) (defined as Lineage-CD34+ CD38− CD90+ CD45RA as shown in flow cytometry gating strategy in FIG. 48) was also investigated. NK cells engineered to express only the FLT3/CD33 bivalent aCAR and NK cells engineered to express both the FLT3/CD33 bivalent aCAR and an EMCN iCAR (SB07412) were tested for their ability to kill leukemia cells, while sparing primary healthy HSCs (in CD34-enriched healthy bone marrow) within an in vitro cytotoxicity assay. For killing of FLT3/CD33-expression leukemia cells, CAR NK cells were co-cultured with the leukemia cells at at an effector:target ratio of 1:2 for approximately 18 hours, then cells were collected, stained, and analyzed by flow cytometry, revealing that the EMCN iCAR did not significantly inhibit the killing of the tumor target cells in comparison to the CAR NK cells without an EMCN iCAR (FIG. 46B). However, when the same CAR NK cells were co-cultured with primary healthy HSCs (within a CD34-enriched healthy bone marrow mixture) at an effector:target ratio of 3:1 for approximately 18 hours, then collected, stained, and analyzed by flow cytometry, the NK cells expressing the FLT3/CD33 bivalent aCAR and an EMCN iCAR demonstrated significantly reduced cytotoxicity against EMCN+ HSCs (FIG. 46A).

Importantly, both OR gate alone (FLT3/CD33 bivalent aCAR) control CAR NK cells and CAR-NK cells that expressed both the FLT3/CD33 bivalent aCAR and an EMCN iCAR (SB07412) killed tumor cells equally, suggesting that when not engaged, the iCAR does not tonically inhibit cytotoxicity. In summary, the killing of healthy EMCN+ HSCs but not the leukemia cells were significantly decreased with those CAR NK cells expressing both the aCAR and the protective iCAR.

In FIG. 46C, a colony forming cell assay was performed to further demonstrate that hematopoietic stem and progenitor cells (HSPCs) populations are maintained in the presence of engineered NK cells transduced with SB07412, which express both the FLT3/CD33 bivalent aCAR and an EMCN iCAR. First, primary healthy HSPCs (CD34-enriched bone marrow) were co-cultured with (or without, as control) CAR-NK cells that were transduced to express the FLT3/CD33 bivalent aCAR and an EMCN iCAR (SB07412) at an effector:target ratio of 1:2 for approximately 18 hours (in triplicate), then cells from each well were collected and 1/10 of the cells were re-plated methylcellulose optimized for the growth and enumeration of hematopoietic stem and progenitor cell colonies, where each colony is a functional readout for a single parental stem/progenitor cell. By counting and scoring colonies from the methylcellulose assay, the co-culture of HSPCs with CAR NK cells possessing the EMCN iCAR did not significantly reduce the number of stem/progenitors (with colony-forming potential) compared to the HSPC-alone control.

As a part of the HSC experiment detailed in FIGS. 46A-46B, the ability of the EMCN iCAR to also reduce killing of progenitor cells was also assessed, specifically multipotent progenitor (MPP) cells (defined as Lineage− CD34+ CD38− CD90− CD45RA− as shown in the flow cytometry gating strategy in FIG. 48). NK cells engineered to express only the FLT3/CD33 bivalent aCAR and NK cells engineered to express both the FLT3/CD33 bivalent aCAR and an EMCN iCAR (SB07412) were tested for their ability to spare primary healthy MPPs (in CD34-enriched healthy bone marrow) within an in vitro cytotoxicity assay. CAR NK cells were co-cultured with primary healthy MPPs (within a CD34-enriched healthy bone marrow mixture) at an effector:target ratio of 3:1 for approximately 18 hours, then collected, stained, and analyzed by flow cytometry, revealing that the NK cells expressing the FLT3/CD33 bivalent aCAR and an EMCN iCAR demonstrated significantly reduced cytotoxicity against both the whole healthy MPP population (FIG. 46D) and healthy EMCN+ MPPs (FIG. 46E).

### NK Cell Phenotyping

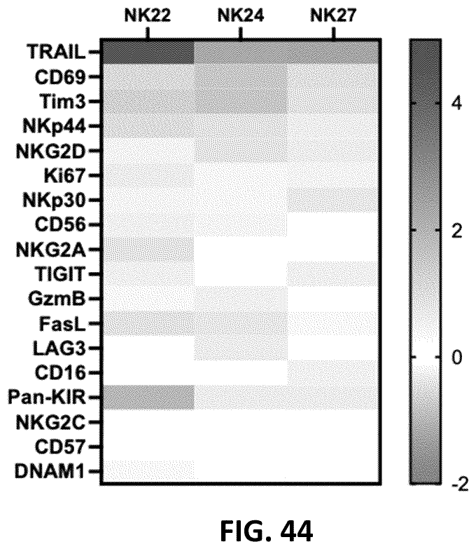

Three different lots of engineered NK cells from separate donors and non-engineered NK cells were analyzed for the expression of phenotypic markers related to NK cell activation, proliferation, and function. Fold change analysis indicated that several markers had elevated expression in engineered NK cells compared to non-engineered NK cells. (FIG. 44). The top seven markers upregulated in engineered NK cells were identified as the cytotoxicity markers TRAIL, NKG2D, NKp30, NKp44, CD69, Ki-67, and Tim-3. Fold expression was calculated using the formula: log 2(gMFI engineered NK cells/gMFI non-engineered NK cells).

Analysis of NK phenotype after co-culture with MV4-11 target cells. Heat map analysis of NK activation marker expression analyzed by flow cytometry (FIG. 44). Each row represents a different phenotypic marker. Markers are rank ordered based on the mean fold change value across all samples.

Other Sequences

HIV-1 protease (SEQ ID NO: 144):
PQVTLWQRPLVTIKIGGQLKEALLDTGADDTVLEEMSLPGRWKPKMIGGIGGFIKVRQY
DQILIEICGHKAIGTVLVGPTPVNIIGRNLLTQIGCTLNF
Angiotensin converting enzyme (ACE) (SEQ ID NO: 156):
MGAASGRRGPGLLLPLPLLLLLPPQPALALDPGLQPGNFSADEAGAQLFAQSYNSSAEQ
VLFQSVAASWAHDTNITAENARRQEEAALLSQEFAEAWGQKAKELYEPIWQNFTDPQL
RRIIGAVRTLGSANLPLAKRQQYNALLSNMSRIYSTAKVCLPNKTATCWSLDPDLTNIL
ASSRSYAMLLFAWEGWHNAAGIPLKPLYEDFTALSNEAYKQDGFTDTGAYWRSWYNS
PTFEDDLEHLYQQLEPLYLNLHAFVRRALHRRYGDRYINLRGPIPAHLLGDMWAQSWE
NIYDMVVPFPDKPNLDVTSTMLQQGWNATHMFRVAEEFFTSLELSPMPPEFWEGSMLE
KPADGREVVCHASAWDFYNRKDFRIKQCTRVTMDQLSTVHHEMGHIQYYLQYKDLPV
SLRRGANPGFHEAIGDVLALSVSTPEHLHKIGLLDRVTNDTESDINYLLKMALEKIAFLP
FGYLVDQWRWGVFSGRTPPSRYNFDWWYLRTKYQGICPPVTRNETHFDAGAKFHVPN
VTPYIRYFVSFVLQFQFHEALCKEAGYEGPLHQCDIYRSTKAGAKLRKVLQAGSSRPWQ
EVLKDMVGLDALDAQPLLKYFQPVTQWLQEQNQQNGEVLGWPEYQWHPPLPDNYPE
GIDLVTDEAEASKFVEEYDRTSQVVWNEYAEANWNYNTNITTETSKILLQKNMQIANH
TLKYGTQARKFDVNQLQNTTIKRIIKKVQDLERAALPAQELEEYNKILLDMETTYSVAT
VCHPNGSCLQLEPDLTNVMATSRKYEDLLWAWEGWRDKAGRAILQFYPKYVELINQA
ARLNGYVDAGDSWRSMYETPSLEQDLERLFQELQPLYLNLHAYVRRALHRHYGAQHI
NLEGPIPAHLLGNMWAQTWSNIYDLVVPFPSAPSMDTTEAMLKQGWTPRRMFKEADD
FFTSLGLLPVPPEFWNKSMLEKPTDGREVVCHASAWDFYNGKDFRIKQCTTVNLEDLV
VAHHEMGHIQYFMQYKDLPVALREGANPGFHEAIGDVLALSVSTPKHLHSLNLLSSEG
GSDEHDINFLMKMALDKIAFIPFSYLVDQWRWRVFDGSITKENYNQEWWSLRLKYQGL
CPPVPRTQGDFDPGAKFHIPSSVPYIRYFVSFIIQFQFHEALCQAAGHTGPLHKCDIYQSK
EAGQRLATAMKLGFSRPWPEAMQLITGQPNMSASAMLSYFKPLLDWLRTENELHGEK
LGWPQYNWTPNSARSEGPLPDSGRVSFLGLDLDAQQARVGQWLLLFLGIALLVATLGL
SQRLFSIRHRSLHRHSHGPQFGSEVELRHS
DENV NS3pro (NS2B/NS3) >sp|P33478|1475-2093 (SEQ ID NO: 157):
SGVLWDTPSPPEVERAVLDDGIYRIMQRGLLGRSQVGVGVFQDGVFHTMWHVTRGAV
LMYQGKRLEPSWASVKKDLISYGGGWRFQGSWNTGEEVQVIAVEPGKNPKNVQTAPG
TFKTPEGEVGAIALDFKPGTSGSPIVNREGKIVGLYGNGVVTTSGTYVSAIAQAKASQEG
PLPEIEDEVFRKRNLTIMDLHPGSGKTRRYLPAIVREAIRRNVRTLILAPTRVVASEMAE
ALKGMPIRYQTTAVKSEHTGKEIVDLMCHATFTMRLLSPVRVPNYNMIIMDEAHFTDP
ASIARRGYISTRVGMGEAAAIFMTATPPGSVEAFPQSNAVIQDEERDIPERSWNSGYEWI
TDFPGKTVWFVPSIKSGNDIANCLRKNGKRVIQLSRKTFDTEYQKTKNNDWDYVVTTD
ISEMGANFRADRVIDPRRCLKPVILKDGPERVILAGPMPVTVASAAQRRGRIGRNQNKE
GDQYVYMGQPLNNDEDHAHWTEAKMLLDNINTPEGIIPALFEPEREKSAAIDGEYRLR
GEARKTFVELMRRGDLPVWLSYKVASEGFQYSDRRWCFDGERNNQVLEENMDVEMW
TKEGERKKLRPRWLDARTYSDPLALREFKEFAAGRR
DENV NS3pro (NS2B/NS3) >sp|P14340|1476-2093 (SEQ ID NO: 158):
AGVLWDVPSPPPVGKAELEDGAYRIKQKGILGYSQIGAGVYKEGTFHTMWHVTRGAV
LMHKGKRIEPSWADVKKDLISYGGGWKLEGEWKEGEEVQVLALEPGKNPRAVQTKPG
LFKTNAGTIGAVSLDFSPGTSGSPIIDKKGKVVGLYGNGVVTRSGAYVSAIAQTEKSIED
NPEIEDDIFRKRKLTIMDLHPGAGKTKRYLPAIVREAIKRGLRTLILAPTRVVAAEMEEA
LRGLPIRYQTPAIRAEHTGREIVDLMCHATFTMRLLSPVRVPNYNLIIMDEAHFTDPASIA
ARGYISTRVEMGEAAGIFMTATPPGSRDPFPQSNAPIMDEEREIPERSWSSGHEWVTDFK
GKTVWFVPSIKAGNDIAACLRKNGKKVIQLSRKTFDSEYVKTRTNDWDFVVTTDISEM
GANFKAERVIDPRRCMKPVILTDGEERVILAGPMPVTHSSAAQRRGRIGRNPKNENDQY
IYMGEPLENDEDCAHWKEAKMLLDNINTPEGIIPSMFEPEREKVDAIDGEYRLRGEARK
TFVDLMRRGDLPVWLAYRVAAEGINYADRRWCFDGIKNNQILEENVEVEIWTKEGER
KKLKPRWLDAKIYSDPLALKEFKEFAAGRK
DENV NS3pro (NS2B/NS3) >sp|Q99D35|1474-2092 (SEQ ID NO: 159):
SGVLWDVPSPPETQKAELEEGVYRIKQQGIFGKTQVGVGVQKEGVFHTMWHVTRGAV
LTHNGKRLEPNWASVKKDLISYGGGWRLSAQWQKGEEVQVIAVEPGKNPKNFQTMPG
IFQTTTGEIGAIALDFKPGTSGSPIINREGKVVGLYGNGVVTKNGGYVSGIAQTNAEPDG
PTPELEEEMFKKRNLTIMDLHPGSGKTRKYLPAIVREAIKRRLRTLILAPTRVVAAEMEE
ALKGLPIRYQTTATKSEHTGREIVDLMCHATFTMRLLSPVRVPNYNLIIMDEAHFTDPAS
IAARGYISTRVGMGEAAAIFMTATPPGTADAFPQSNAPIQDEERDIPERSWNSGNEWITD
FVGKTVWFVPSIKAGNDIANCLRKNGKKVIQLSRKTFDTEYQKTKLNDWDFVVTTDISE
MGANFKADRVIDPRRCLKPVILTDGPERVILAGPMPVTVASAAQRRGRVGRNPQKEND
QYIFMGQPLNKDEDHAHWTEAKMLLDNINTPEGIIPALFEPEREKSAAIDGEYRLKGESR
KTFVELMRRGDLPVWLAHKVASEGIKYTDRKWCFDGERNNQILEENMDVEIWTKEGE
KKKLRPRWLDARTYSDPLALKEFKDFAAGRK
DENV NS3pro (NS2B/NS3) >sp/Q5UCB8|1475-2092 (SEQ ID NO: 160):
SGALWDVPSPAATQKAALSEGVYRIMQRGLFGKTQVGVGIHIEGVFHTMWHVTRGSVI
CHETGRLEPSWADVRNDMISYGGGWRLGDKWDKEEDVQVLAIEPGKNPKHVQTKPGL
FKTLTGEIGAVTLDFKPGTSGSPIINRKGKVIGLYGNGVVTKSGDYVSAITQAERIGEPDY
EVDEDIFRKKRLTIMDLHPGAGKTKRILPSIVREALKRRLRTLILAPTRVVAAEMEEALR
GLPIRYQTPAVKSEHTGREIVDLMCHATFTTRLLSSTRVPNYNLIVMDEAHFTDPSSVAA
RGYISTRVEMGEAAAIFMTATPPGTTDPFPQSNSPIEDIEREIPERSWNTGFDWITDYQGK
TVWFVPSIKAGNDIANCLRKSGKKVIQLSRKTFDTEYPKTKLTDWDFVVTTDISEMGAN
FRAGRVIDPRRCLKPVILPDGPERVILAGPIPVTPASAAQRRGRIGRNPAQEDDQYVESG
DPLKNDEDHAHWTEAKMLLDNIYTPEGIIPTLFGPEREKTQAIDGEFRLRGEQRKTFVEL
MRRGDLPVWLSYKVASAGISYKDREWCFTGERNNQILEENMEVEIWTREGEKKKLRPK
WLDARVYADPMALKDFKEFASGRK
PEST (two copies of residues 277-307 of human IKBa; SEQ ID NO: 161):
LQMLPESEDEESYDTESEFTEFTEDELPYDDGSLQMLPESEDEESYDTESEFTEFTEDELP
YDD
GRR(residues 352-408 of human p105; SEQ ID NO: 162):
EIKDKEEVQRKRQKLMPNFSDSFGGGSGAGAGGGGMFGSGGGGGGTGSTGPGYSFPH
DRR(residues 210-295 of yeast Cdc34; SEQ ID NO: 163):
IDDENGSVILQDDDYDDGNNHIPFEDDDVYNYNDNDDDDERIEFEDDDDDDDDSIDND
SVMDRKQPHKAEDESEDVEDVERVSKKD
SNS (tandem repeat of SP2 and NB (SP2-NB-SP2 of influenza A or influenza B; e.g., SEQ ID NO: 164):
PESMREEYRKEGSKRIKCPDCEPFCNKRGSPESMREEYRKE
RPB (four copies of residues 1688-1702 of yeast RPB; SEQ ID NO: 165):
RSYSPTSPNYSPTSPSGSYSPTSPNYSPTSPSGGSRSYSPTSPNYSPTSPSGSYSPTSPNYSP
TSPSG
SPmix (tandem repeat of SP1 and SP2 (SP2-SP1-SP2-SP1-SP2 of influenza A virus M2
protein; SEQ ID NO: 166):
PESMREEYRKEGSSLLTEVETPGSPESMREEYRKEGSSLLTEVETPGSPESMREEYRKE
NS2 (three copies of residues 79-93 of influenza A virus NS protein; SEQ ID NO: 167):
LIEEVRHRLKTTENSGSLIEEVRHRLKTTENSGSLIEEVRHRLKTTENSGS
ODC (residues 106-142 of ornithine decarboxylase; SEQ ID NO: 168):
FPPEVEEQDDGTLPMSCAQESGMDRHPAACASARINV
mouse ODC (residues 422-461; SEQ ID NO: 169):
SHGFPPEVEEQAAGTLPMSCAQESGMDRHPAACASARINV
SB03513 (SEQ ID NO: 210)
AATMDWTWILFLVAAATRVHSNWVNVISDLKKIEDLIQSMHIDATLYTESDVHPSCKV
TAMKCFLLELQVISLESGDASIHDTVENLIILANNSLSSNGNVTESGCKECEELEEKNIKE
FLQSFVHIVQMFINTSDEPHYSQRRLLPSWAITLISVNGIFVICCLTYCFAPRCRERRRNER
LRRESVRPV
SB03514 (SEQ ID NO: 211)
AATMDWTWILFLVAAATRVHSNWVNVISDLKKIEDLIQSMHIDATLYTESDVHPSCKV
TAMKCFLLELQVISLESGDASIHDTVENLIILANNSLSSNGNVTESGCKECEELEEKNIKE
FLQSFVHIVQMFINTSVTPEPIFSLILLPSWAITLISVNGIFVICCLTYCFAPRCRERRRNER
LRRESVRPV
SB03515 (SEQ ID NO: 212)
AATMDWTWILFLVAAATRVHSNWVNVISDLKKIEDLIQSMHIDATLYTESDVHPSCKV
TAMKCFLLELQVISLESGDASIHDTVENLIILANNSLSSNGNVTESGCKECEELEEKNIKE
FLQSFVHIVQMFINTSDEPHYSQRRSGGGGSGGGGSGGGGSGGGGSGGGSLQLLPSWAI
TLISVNGIFVICCLTYCFAPRCRERRRNERLRRESVRPV
SB03516 (SEQ ID NO: 213)
AATMDWTWILFLVAAATRVHSNWVNVISDLKKIEDLIQSMHIDATLYTESDVHPSCKV
TAMKCFLLELQVISLESGDASIHDTVENLIILANNSLSSNGNVTESGCKECEELEEKNIKE
FLQSFVHIVQMFINTSVTPEPIFSLISGGGGSGGGGSGGGGSGGGGSGGGSLQLLPSWAIT
LISVNGIFVICCLTYCFAPRCRERRRNERLRRESVRPV
SB03517 (SEQ ID NO: 214)
AATMDWTWILFLVAAATRVHSNWVNVISDLKKIEDLIQSMHIDATLYTESDVHPSCKV
TAMKCFLLELQVISLESGDASIHDTVENLIILANNSLSSNGNVTESGCKECEELEEKNIKE
FLQSFVHIVQMFINTSSGGGGSGGGGSGDEPHYSQRRGGGSGGGGSGGGSLQLLPSWAI
TLISVNGIFVICCLTYCFAPRCRERRRNERLRRESVRPV
SB03518 (SEQ ID NO: 215)
AATMDWTWILFLVAAATRVHSNWVNVISDLKKIEDLIQSMHIDATLYTESDVHPSCKV
TAMKCFLLELQVISLESGDASIHDTVENLIILANNSLSSNGNVTESGCKECEELEEKNIKE
FLQSFVHIVQMFINTSSGGGGSGGGGSGVTPEPIFSLIGGGSGGGGSGGGSLQLLPSWAIT
LISVNGIFVICCLTYCFAPRCRERRRNERLRRESVRPV
SB03519 (SEQ ID NO: 216)
AATMDWTWILFLVAAATRVHSNWVNVISDLKKIEDLIQSMHIDATLYTESDVHPSCKV
TAMKCFLLELQVISLESGDASIHDTVENLIILANNSLSSNGNVTESGCKECEELEEKNIKE
FLQSFVHIVQMFINTSSGGGGSGGGGSGGGGSGGGGSGGGSLQDEPHYSQRRLLPSWAI
TLISVNGIFVICCLTYCFAPRCRERRRNERLRRESVRPV
SB03520 (SEQ ID NO: 217)
AATMDWTWILFLVAAATRVHSNWVNVISDLKKIEDLIQSMHIDATLYTESDVHPSCKV
TAMKCFLLELQVISLESGDASIHDTVENLIILANNSLSSNGNVTESGCKECEELEEKNIKE
FLQSFVHIVQMFINTSSGGGGSGGGGSGGGGSGGGGSGGGSLQVTPEPIFSLILLPSWAIT
LISVNGIFVICCLTYCFAPRCRERRRNERLRRESVRPV
5X NFAT minADEp promoter (SEQ ID NO: 218)
GGGACTTTCCACTGGGGACTTTCCACTGGGGACTTTCCACTGGGGACTTTCCACTGG
GGACTTTCCACTCCTGCAGGagctGGCGCGCCAGACGCTAGCGGGGGGCTATAAAAG
GGGGTGGGGGCGTTCGTCCTCACTCT
SB06342; SINvec SV40 (SEQ ID NO: 425)
aagcttgaattcgagcttgcatgcctgcaggtcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggata
gcggtttgactcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagagctcaataaaagagcccaca
acccctcactcggegcgccagtcctccgattgactgagtcgcccgggtacccgtgtatccaataaaccctcttgcagttgcatccgacttgtg
gtctcgctgttccttgggagggtctcctctgagtgattgactacccgtcagcgggggtctttcatttgggggctcgtccgagatcgggagacc
cctgcccagggaccaccgacccaccaccgggaggtaagctggccagcaacttatctgtgtctgtccgattgtctagtgtctatgactgatttt
atgcgcctgcgtcggtactagttagctaactagctctgtatctggcggacccgtggtggaactgacgagttcggaacacccggccgcaacc
ctgggagacgtcccagggacttcgggggccgtttttgtggcccgacctgagtcctaaaatcccgatcgtttaggactctttggtgcaccccc
cttagaggagggatatgtggttctggtaggagacgagaacctaaaacagttcccgcctccgtctgaatttttgctttcggtttgggaccgaag
ccgcgccgcgcgtcttgtctgctgcagcatcgttctgtgttgtctctgtctgactgtgtttctgtatttgtctgaaaatatgggccccccctcgag
CTGTGGAATGTGTGTCAGTTAGGGTGTGGAAAGTCCCCAGGCTCCCCAGCAGGCAG
AAGTATGCAAAGCATGCATCTCAATTAGTCAGCAACCAGGTGTGGAAAGTCCCCAG
GCTCCCCAGCAGGCAGAAGTATGCAAAGCATGCATCTCAATTAGTCAGCAACCATA
GTCCCGCCCCTAACTCCGCCCATCCCGCCCCTAACTCCGCCCAGTTCCGCCCATTCT
CCGCCCCATGGCTGACTAATTTTTTTTATTTATGCAGAGGCCGAGGCCGCCTCTGCC
TCTGAGCTATTCCAGAAGTAGTGAGGAGGCTTTTTTGGAGGCCTAGGCTTTTGCAAA
AAGCTGGATCCGCGTCAAGTGGAGCAAGGCAGGTGGACAGTCTCGAggccgccaccATG
GACTGGACTTGGATACTCTTTCTGGTCGCTGCCGCCACACGGGTGCACTCTAATTGG
GTCAACGTGATCAGCGACCTGAAGAAGATCGAGGACCTGATCCAGAGCATGCACAT
CGACGCCACACTGTACACCGAGTCCGATGTGCACCCTAGCTGCAAAGTGACCGCCA
TGAAGTGCTTTCTGCTGGAACTGCAAGTGATCAGCCTGGAAAGCGGCGACGCCAGC
ATCCACGATACCGTGGAAAATCTGATCATCCTGGCCAACAACAGCCTGTCCAGCAA
CGGCAATGTGACCGAGAGCGGCTGCAAAGAGTGCGAGGAACTGGAAGAGAAGAAC
ATCAAAGAGTTTCTGCAGAGCTTCGTCCACATCGTGCAGATGTTCATCAACACCTCA
TCAGGCGGCGGTGGTAGTGGAGGCGGAGGCTCAGGCGTGACCCCTGAGCCTATCTT
CAGCCTGATCGGCGGAGGTTCCGGAGGTGGCGGTTCCGGCGGAGGATCTCTTCAAT
TGCTGCCTAGCTGGGCCATCACACTGATCTCCGTGAACGGCATCTTCGTGATCTGCT
GCCTGACCTACTGCTTCGCCCCTAGATGCAGAGAGCGGAGAAGAAACGAGCGGCTG
AGAAGAGAAAGCGTGCGGCCTGTGGGTAGCGGCCAGTGTACCAACTACGCCCTGCT
GAAACTGGCCGGCGACGTGGAATCTAATCCTGGACCTGGATCTGGCGAGGGACGCG
GGAGTCTACTGACGTGTGGAGACGTGGAGGAAAACCCTGGACCTATGCTGCTGCTG
GTTACATCTCTGCTGCTGTGCGAGCTGCCCCATCCTGCCTTTCTGCTTATTCCTGAAG
TTCAGCTGGTTCAGAGCGGAGCCGAAGTGAAGAAACCTGGCAGCAGCGTGAAGGT
GTCCTGCAAAGCTTCTGGCGGCACCTTCAGCAGCTACGCCATCTCTTGGGTTCGACA
GGCACCTGGACAGGGCCTCGAGTGGATGGGAGGAATCATCCCTATCTTCGGCACCG
CCAATTACGCCCAGAAATTTCAGGGAAGAGTGACCATCACCGCCGACAAGAGCACC
AGCACCGCCTACATGGAACTGAGCAGCCTGAGAAGCGAGGATACCGCTGTGTATTA
CTGCGCCACATTCGCCCTGTTCGGCTTCAGAGAGCAGGCCTTCGATATCTGGGGCCA
GGGCACAACCGTGACAGTTTCATCTGGCGGCGGTGGCTCTCAGGTTCAGCTTGTGCA
ATCTGGCGCTGAAGTGAAAAAGCCCGGCTCCTCCGTGAAAGTGTCTTGTAAAGCCA
GCGGCTACACCTTTACCGACTACAATATGCATTGGGTCCGCCAGGCACCAGGCCAG
GGACTTGAATGGATCGGCTACATCTACCCCTACAACGGCGGCACCGGCTACAACCA
GAAGTTCAAGTCCAAGGCCACAATCACAGCCGACGAGTCCACCAATACTGCTTATA
TGGAACTGTCTAGCCTTCGGAGCGAGGACACAGCCGTGTACTATTGCGCTAGAGGC
AGACCCGCCATGGACTATTGGGGACAGGGAACCCTGGTCACAGTGTCCAGCGGCTC
TACATCTGGCTCTGGCAAACCTGGAAGCGGCGAGGGATCTACCAAGGGCGACATCC
AGATGACACAGAGCCCTAGTAGCCTGAGCGCCAGCGTGGGAGACAGAGTGACAAT
TACCTGTCGGGCCAGCGAGAGCGTGGACAACTACGGCATCAGCTTCATGAACTGGT
TCCAGCAGAAGCCTGGCAAGGCCCCTAAGCTGCTGATCTATGCCGCCAGCAATCAA
GGCAGCGGAGTGCCCTCAAGATTCAGCGGAAGCGGCTCCGGCACCGACTTTACCCT
GACAATCAGCTCCCTGCAGCCTGACGACTTCGCCACCTACTACTGCCAGCAGAGCA
AAGAGGTGCCCTGGACATTCGGACAGGGCACCAAGGTGGAAATCAAAGGTGGCGG
CGGATCCGATATTCAGATGACTCAGTCCCCAAGCAGCCTGTCAGCCTCCGTGGGCG
ATAGAGTCACCATTACATGCAGAGCCAGCCAGTCCATCTCCAGCTACCTGAACTGG
TATCAGCAAAAACCCGGAAAGGCTCCAAAGCTCCTCATCTACGCCGCTTCTAGCCT
GCAGTCTGGCGTGCCATCTAGATTCTCTGGCTCCGGAAGCGGGACCGACTTCACACT
GACCATATCTTCTCTGCAGCCCGAAGATCTGGCCACGTACTATTGTCAGCAGTCCTA
CAGCACCCCTTTCACCTTCGGACCCGGAACAAAGGTGGACATTAAGGCCCTGAGCA
ACAGCATCATGTACTTCAGCCACTTCGTGCCCGTGTTTCTGCCCGCCAAGCCTACAA
CAACCCCTGCTCCTAGACCACCTACACCAGCTCCTACAATCGCCAGCCAGCCTCTGT
CTCTGAGGCCCGAAGCTTGTAGACCAGCTGCTGGCGGAGCCGTGCATACAAGAGGA
CTGGATTTTGCCTGCGACATCTATATCTGGGCCCCTCTGGCTGGCACATGCGGAGTT
CTGCTGCTCAGCCTGGTCATCACCCTGTACTGCAACCACAGAAGAAGCAAGCGGAG
CAGACTGCTGCACAGCGACTACATGAACATGACCCCTAGACGGCCCGGACCTACCA
GAAAGCACTACCAGCCTTACGCTCCTCCTAGAGATTTCGCCGCCTACCGGTCCAGAG
TGAAGTTCAGCAGGAGCGCAGACGCCCCCGCGTACAAGCAGGGCCAGAACCAGCT
CTATAACGAGCTCAATCTAGGACGAAGAGAGGAGTACGATGTTTTGGACAAGAGAC
GTGGCCGGGACCCTGAGATGGGGGGAAAGCCGAGAAGGAAGAACCCTCAGGAAGG
CCTGTACAATGAACTGCAGAAAGATAAGATGGCGGAGGCCTACAGTGAGATTGGG
ATGAAAGGCGAGCGCCGGAGGGGCAAGGGGCACGATGGCCTTTACCAGGGTCTCA
GTACAGCCACCAAGGACACCTACGACGCCCTTCACATGCAGGCCCTGCCCCCTCGC
GGTTCTGGCCAGTGCACAAATTATGCACTGCTGAAGCTCGCCGGGGATGTCGAGAG
TAACCCAGGACCTGGAAGCGGAGAAGGTCGTGGTAGTCTACTAACGTGTGGTGATG
TAGAAGAAAATCCTGGACCTATGGCTCTGCCCGTGACAGCTTTGCTGCTGCCTTTGG
CACTGCTGCTGCATGCTGCTAGACCAGAGGTGCAGCTGGTTGAATCTGGCGGAGGA
CTGGTTCAGCCTGGCGGATCTCTGAGACTGTCTTGTGCCGCCAGCGGCTTCACCTTC
AGCAGATACGATATGCACTGGGTCCGACAGGCCCCTGGCAAAGGACTTGAATGGGT
GTCCGTGATCTGGGGCAACGGCAACACACACTACCACAGCGCCCTGAAGTCCCGGT
TCACCATCTCCAGAGACAACAGCAAGAACACCCTGTACCTGCAGATGAACAGCCTG
AGAGCCGAGGACACCGCCGTGTACTACTGCACCCTGAGAATCAAGGATTGGGGCCA
GGGCACCATGGTCACCGTTTCTTCTGGAGGCGGAGGATCTGGTGGCGGAGGAAGTG
GCGGAGGCGGTTCTGACGTGGTCATGACACAGAGCCCTCTGAGCCTGCCTGTGACA
CTGGGACAGCCTGCCAGCATCAGCTGCAAGTCTAGCCAGTCTCTGGTGGCCAGCGA
CGAGAACACCTACCTGAACTGGTTCCAGCAGAGGCCCGGACAGTCTCCTAGACGGC
TGATCTACCAGGTGTCCAAGCTGGATAGCGGCGTGCCCGATAGATTTTCTGGCAGC
GGCTCTGGCACCGACTTCACCCTGAAGATCAGCAGAGTGGAAGCCGAGGACGTGGG
CGTGTACTACTGTCTGCAAGGCATCCATCTGCCTTGGACCTTTGGCCAGGGCACAAA
GCTGGAAATCAAGAATGGCGCTGCAACAACAACACCCGCACCTCGGCCTCCAACTC
CAGCTCCAACAATTGCACTGCAACCCCTGAGTCTGAGGCCCGAGGCCTGTAGGCCA
GCAGCTGGCGGAGCTGTTCACACTAGAGGCCTGGACTTTGCCTGTGACGTGATCGG
CATTCTGGTCGCCGTGATCCTGCTCCTGTTGCTCCTGCTGCTTCTGTTCCTGATCCTG
CGGCACAGAAGGCAGGGCAAGCACTGGACAAGCACCCAGAGAAAGGCCGACTTTC
AGCATCCTGCTGGCGCCGTTGGACCTGAGCCTACAGATAGAGGACTGCAGTGGCGG
TCTAGCCCTGCCGCTGATGCCCAAGAGGAAAATCTTTACGCCGCCGTGAAGCACAC
CCAGCCTGAGGATGGCGTGGAAATGGACACCAGATCTCCCCACGATGAGGACCCTC
AGGCCGTGACATACGCAGAAGTGAAGCACTCCAGACCTCGGAGAGAGATGGCAAG
CCCTCCATCTCCTCTGAGCGGCGAGTTCCTGGACACCAAAGACAGACAGGCCGAAG
AGGACAGACAGATGGATACCGAAGCCGCCGCTTCTGAAGCCCCACAGGATGTGACA
TATGCCCAGCTGCATAGCCTGACACTGCGGAGAGAAGCCACAGAGCCTCCACCTTC
TCAAGAAGGCCCATCTCCTGCCGTGCCTTCCATCTATGCCACTCTGGCCATTCACtaaT
gAggatccggattagtccaatttgttaaagacaggatgggctgcaggaattccgataatcaacctctggattacaaaatttgtgaaagattga
ctggtattcttaactatgttgctccttttacgctatgtggatacgctgctttaatgcctttgtatcatgctattgcttcccgtatggctttcattttctcct
ccttgtataaatcctggttgctgtctctttatgaggagttgtggcccgttgtcaggcaacgtggcgtggtgtgcactgtgtttgctgacgcaacc
cccactggttggggcattgccaccacctgtcagctcctttccgggactttcgctttccccctccctattgccacggeggaactcategcegcc
tgccttgcccgctgctggacaggggctcggctgttgggcactgacaattccgtggtgttgtcggggaagctgacgtcctttccatggctgct
cgcctgtgttgccacctggattctgcgcgggacgtccttctgctacgtcccttcggccctcaatccageggaccttccttcccgcggcctgct
gccggctctgcggcctcttccgcgtcttcgccttcgccctcagacgagtcggatctccctttgggccgcctccccgcctggagaattcgatat
cagtggtccaggctctagttttgactcaacaatatcaccagctgaagcctatagagtacgagccatagataaaataaaagattttatttagtctc
cagaaaaaggggggaatgaaagaccccacctgtaggtttggcaagctagcaataaaagagcccacaacccctcactcggggcgccagt
cctccgattgactgagtcgcccggccgcttcgagcagacatgataagatacattgatgagtttggacaaaccacaactagaatgcagtgaa
aaaaatgctttatttgtgaaatttgtgatgctattgctttatttgtaaccattataagctgcaataaacaagttaacaacaacaattgcattcattttat
gtttcaggttcagggggagatgtgggaggttttttaaagcaagtaaaacctctacaaatgtggtaaaatcgataaggatcgggtacccgtgta
tccaataaaccctcttgcagttgcatccgacttgtggtctcgctgttccttgggagggtctcctctgagtgattgactacccgtcagcgggggt
ctttcacacatgcagcatgtatcaaaattaatttggttttttttcttaagctgtgccttctagttgccagccatctgttgtttgcccctcccccgtgcc
ttccttgaccctggaaggtgccactcccactgtcctttcctaataaaatgaggaaattgcatcgcattgtctgagtaggtgtcattctattctggg
gggtggggggggcaggacagcaagggggaggattgggaagacaatagcaggcatgctggggatgcggtgggctctatggagatcc
cgcggtacctcgcgaatgcatctagatccaatggcctttttggcccagacatgataagatacattgatgagtttggacaaaccacaactagaa
tgcagtgaaaaaaatgctttatttgtgaaatttgtgatgctattgctttatttgtaaccattataagctgcaataaacaagttgcggccgcttagcc
ctcccacacataaccagagggcagcaattcacgaatcccaactgccgtcggctgtccatcactgtccttcactatggctttgatcccaggatg
cagatcgagaagcacctgtcggcaccgtccgcaggggctcaagatgcccctgttctcatttccgatcgcgacgatacaagtcaggttgcca
gctgccgcagcagcagcagtgcccagcaccacgagttctgcacaaggtcccccagtaaaatgatatacattgacaccagtgaagatgcg
gccgtcgctagagagagctgcgctggcgacgctgtagtcttcagagatggggatgctgttgattgtagccgttgctctttcaatgagggtgg
attcttcttgagacaaaggcttggccatgcggccgccgctcggtgttcgaggccacacgcgtcaccttaatatgcgaagtggacctcggac
cgcgccgccccgactgcatctgcgtgttcgaattcgccaatgacaagacgctgggcggggtttgtgtcatcatagaactaaagacatgcaa
atatatttcttccggggggtaccggcctttttggccATTGGatcggatctggccaaaaaggcccttaagtatttacattaaatggccatagtacttaaa
gttacattggcttccttgaaataaacatggagtattcagaatgtgtcataaatatttctaattttaagatagtatctccattggctttctactttttcttt
tatttttttttgtcctctgtcttccatttgttgttgttgttgtttgtttgtttgtttgttggttggttggttaatttttttttaaagatcctacactatagt
tcaagctagactattagctactctgtaacccagggtgaccttgaagtcatgggtagcctgctgttttagccttcccacatctaagattacaggta
tgagctatcatttttggtatattgattgattgattgattgatgtgtgtgtgtgtgattgtgtttgtgtgtgtgaTtgtgTaTatgtgtgtatggTtgtg
tgtgaTtgtgtgtatgtatgTTtgtgtgtgaTtgTgtgtgtgtgaTtgtgcatgtgtgtgtgtgtgaTtgtgtTtatgtgtatgaTtgtgtgtg
tgtgtgtgtgtgtgtgtgtgtgtgtgtgtgtgtgtgtgttgtgTaTaTatatttatggtagtgagagGcaacgctccggctcaggtgtcaggtt
ggtttttgagacagagtctttcacttagcttggaattcactggccgtcgttttacaacgtcgtgactgggaaaaccctggcgttacccaacttaa
tcgccttgcagcacatccccctttcgccagctggcgtaatagcgaagaggcccgcaccgatcgcccttcccaacagttgcgcagcctgaat
ggcgaatggcgcctgatgcggtattttctccttacgcatctgtgcggtatttcacaccgcatatggtgcactctcagtacaatctgctctgatgc
cgcatagttaagccagccccgacacccgccaacacccgctgacgcgccctgacgggcttgtctgctcccggcatccgcttacagacaag
ctgtgaccgtctccgggagctgcatgtgtcagaggttttcaccgtcatcaccgaaacgcgcgagacgaaagggcctcgtgatacgcctattt
ttataggttaatgtcatgataataatggtttcttagacgtcaggtggcacttttcggggaaatgtgcgcggaacccctatttgtttatttttctaaat
acattcaaatatgtatccgctcatgagacaataaccctgataaatgcttcaataatattgaaaaaggaagagtatgagtattcaacatttccgtgt
cgcccttattcccttttttgcggcattttgccttcctgtttttgctcacccagaaacgctggtgaaagtaaaagatgctgaagatcagttgggtgc
acgagtgggttacatcgaactggatctcaacagcggtaagatccttgagagttttcgccccgaagaacgttttccaatgatgagcacttttaaa
gttctgctatgtggcgcggtattatcccgtattgacgccgggcaagagcaactcggtcgccgcatacactattctcagaatgacttggttgag
tactcaccagtcacagaaaagcatcttacggatggcatgacagtaagagaattatgcagtgctgccataaccatgagtgataacactgcgg
ccaacttacttctgacaacgatcggaggaccgaaggagctaaccgcttttttgcacaacatgggggatcatgtaactcgccttgatcgttggg
aaccggagctgaatgaagccataccaaacgacgagcgtgacaccacgatgcctgtagcaatggcaacaacgttgcgcaaactattaactg
gcgaactacttactctagcttcccggcaacaattaatagactggatggaggcggataaagttgcaggaccacttctgcgctcggcccttccg
gctggctggtttattgctgataaatctggagccggtgagcgtgggtctcgcggtatcattgcagcactggggccagatggtaagccctccc
gtatcgtagttatctacacgacggggagtcaggcaactatggatgaacgaaatagacagatcgctgagataggtgcctcactgattaagcat
tggtaactgtcagaccaagtttactcatatatactttagattgatttaaaacttcatttttaatttaaaaggatctaggtgaagatcctttttgataatct
catgaccaaaatcccttaacgtgagttttcgttccactgagcgtcagaccccgtagaaaagatcaaaggatcttcttgagatcctttttttctgcg
cgtaatctgctgcttgcaaacaaaaaaaccaccgctaccagcggtggtttgtttgccggatcaagagctaccaactctttttccgaaggtaact
ggcttcagcagagcgcagataccaaatactgtccttctagtgtagccgtagttaggccaccacttcaagaactctgtagcaccgcctacata
cctcgctctgctaatcctgttaccagtggctgctgccagtggcgataagtcgtgtcttaccgggttggactcaagacgatagttaccggataa
ggcgcagcggtcgggctgaacggggggttcgtgcacacagcccagcttggagcgaacgacctacaccgaactgagatacctacagcgt
gagctatgagaaagcgccacgcttcccgaagggagaaaggcggacaggtatccggtaagcggcagggtcggaacaggagagcgcac
gagggagcttccagggggaaacgcctggtatctttatagtcctgtcgggtttcgccacctctgacttgagcgtcgatttttgtgatgctcgtca
ggggggcggagcctatggaaaaacgccagcaacgcggcctttttacggttcctggccttttgctggccttttgctcacatgttctttcctgcgt
tatcccctgattctgtggataaccgtattaccgcctttgagtgagctgataccgctcgccgcagccgaacgaccgagcgcagcgagtcagt
gagcgaggaagcggaagagcgcccaatacgcaaaccgcctctccccgcgcgttggccgattcattaatgcagctggcacgacaggtttc
ccgactggaaagcgggcagtgagcgcaacgcaattaatgtgagttagctcactcattaggcaccccaggctttacactttatgcttccggct
cgtatgttgtgtggaattgtgagcggataacaatttcacacaggaaacagctatgaccatgattacgcc
SB06463; SINvec SV40 (SEQ ID NO: 426)
aagcttgaattcgagcttgcatgcctgcaggtcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggata
gcggtttgactcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagagctcaataaaagagcccaca
acccctcactcggcgcgccagtcctccgattgactgagtcgcccgggtacccgtgtatccaataaaccctcttgcagttgcatccgacttgtg
gtctcgctgttccttgggagggtctcctctgagtgattgactacccgtcagcgggggtctttcatttgggggctcgtccgagatcgggagacc
cctgcccagggaccaccgacccaccaccgggaggtaagctggccagcaacttatctgtgtctgtccgattgtctagtgtctatgactgatttt
atgcgcctgcgtcggtactagttagctaactagctctgtatctggcggacccgtggtggaactgacgagttcggaacacccggccgcaacc
ctgggagacgtcccagggacttcgggggccgtttttgtggcccgacctgagtcctaaaatcccgatcgtttaggactctttggtgcaccccc
cttagaggagggatatgtggttctggtaggagacgagaacctaaaacagttcccgcctccgtctgaatttttgctttcggtttgggaccgaag
ccgcgccgcgcgtcttgtctgctgcagcatcgttctgtgttgtctctgtctgactgtgtttctgtatttgtctgaaaatatgggccccccctcgag
CTGTGGAATGTGTGTCAGTTAGGGTGTGGAAAGTCCCCAGGCTCCCCAGCAGGCAG
AAGTATGCAAAGCATGCATCTCAATTAGTCAGCAACCAGGTGTGGAAAGTCCCCAG
GCTCCCCAGCAGGCAGAAGTATGCAAAGCATGCATCTCAATTAGTCAGCAACCATA
GTCCCGCCCCTAACTCCGCCCATCCCGCCCCTAACTCCGCCCAGTTCCGCCCATTCT
CCGCCCCATGGCTGACTAATTTTTTTTATTTATGCAGAGGCCGAGGCCGCCTCTGCC
TCTGAGCTATTCCAGAAGTAGTGAGGAGGCTTTTTTGGAGGCCTAGGCTTTTGCAAA
AAGCTGGATCCGCGTCAAGTGGAGCAAGGCAGGTGGACAGTCTCGAggccgccaccATG
GACTGGACTTGGATACTCTTTCTGGTCGCTGCCGCCACACGGGTGCACTCTAATTGG
GTCAACGTGATCAGCGACCTGAAGAAGATCGAGGACCTGATCCAGAGCATGCACAT
CGACGCCACACTGTACACCGAGTCCGATGTGCACCCTAGCTGCAAAGTGACCGCCA
TGAAGTGCTTTCTGCTGGAACTGCAAGTGATCAGCCTGGAAAGCGGCGACGCCAGC
ATCCACGATACCGTGGAAAATCTGATCATCCTGGCCAACAACAGCCTGTCCAGCAA
CGGCAATGTGACCGAGAGCGGCTGCAAAGAGTGCGAGGAACTGGAAGAGAAGAAC
ATCAAAGAGTTTCTGCAGAGCTTCGTCCACATCGTGCAGATGTTCATCAACACCTCA
TCAGGCGGCGGTGGTAGTGGAGGCGGAGGCTCAGGCGTGACCCCTGAGCCTATCTT
CAGCCTGATCGGCGGAGGTTCCGGAGGTGGCGGTTCCGGCGGAGGATCTCTTCAAT
TGCTGCCTAGCTGGGCCATCACACTGATCTCCGTGAACGGCATCTTCGTGATCTGCT
GCCTGACCTACTGCTTCGCCCCTAGATGCAGAGAGCGGAGAAGAAACGAGCGGCTG
AGAAGAGAAAGCGTGCGGCCTGTGGGTAGCGGCCAGTGTACCAACTACGCCCTGCT
GAAACTGGCCGGCGACGTGGAATCTAATCCTGGACCTGGATCTGGCGAGGGACGCG
GGAGTCTACTGACGTGTGGAGACGTGGAGGAAAACCCTGGACCTATGCTGCTGCTG
GTTACATCTCTGCTGCTGTGCGAGCTGCCCCATCCTGCCTTTCTGCTTATTCCTGAAG
TTCAGCTGGTTCAGAGCGGAGCCGAAGTGAAGAAACCTGGCAGCAGCGTGAAGGT
GTCCTGCAAAGCTTCTGGCGGCACCTTCAGCAGCTACGCCATCTCTTGGGTTCGACA
GGCACCTGGACAGGGCCTCGAGTGGATGGGAGGAATCATCCCTATCTTCGGCACCG
CCAATTACGCCCAGAAATTTCAGGGAAGAGTGACCATCACCGCCGACAAGAGCACC
AGCACCGCCTACATGGAACTGAGCAGCCTGAGAAGCGAGGATACCGCTGTGTATTA
CTGCGCCACATTCGCCCTGTTCGGCTTCAGAGAGCAGGCCTTCGATATCTGGGGCCA
GGGCACAACCGTGACAGTTTCATCTGGCGGCGGTGGCTCTCAGGTTCAGCTTGTGCA
ATCTGGCGCTGAAGTGAAAAAGCCCGGCTCCTCCGTGAAAGTGTCTTGTAAAGCCA
GCGGCTACACCTTTACCGACTACAATATGCATTGGGTCCGCCAGGCACCAGGCCAG
GGACTTGAATGGATCGGCTACATCTACCCCTACAACGGCGGCACCGGCTACAACCA
GAAGTTCAAGTCCAAGGCCACAATCACAGCCGACGAGTCCACCAATACTGCTTATA
TGGAACTGTCTAGCCTTCGGAGCGAGGACACAGCCGTGTACTATTGCGCTAGAGGC
AGACCCGCCATGGACTATTGGGGACAGGGAACCCTGGTCACAGTGTCCAGCGGCTC
TACATCTGGCTCTGGCAAACCTGGAAGCGGCGAGGGATCTACCAAGGGCGACATCC
AGATGACACAGAGCCCTAGTAGCCTGAGCGCCAGCGTGGGAGACAGAGTGACAAT
TACCTGTCGGGCCAGCGAGAGCGTGGACAACTACGGCATCAGCTTCATGAACTGGT
TCCAGCAGAAGCCTGGCAAGGCCCCTAAGCTGCTGATCTATGCCGCCAGCAATCAA
GGCAGCGGAGTGCCCTCAAGATTCAGCGGAAGCGGCTCCGGCACCGACTTTACCCT
GACAATCAGCTCCCTGCAGCCTGACGACTTCGCCACCTACTACTGCCAGCAGAGCA
AAGAGGTGCCCTGGACATTCGGACAGGGCACCAAGGTGGAAATCAAAGGTGGCGG
CGGATCCGATATTCAGATGACTCAGTCCCCAAGCAGCCTGTCAGCCTCCGTGGGCG
ATAGAGTCACCATTACATGCAGAGCCAGCCAGTCCATCTCCAGCTACCTGAACTGG
TATCAGCAAAAACCCGGAAAGGCTCCAAAGCTCCTCATCTACGCCGCTTCTAGCCT
GCAGTCTGGCGTGCCATCTAGATTCTCTGGCTCCGGAAGCGGGACCGACTTCACACT
GACCATATCTTCTCTGCAGCCCGAAGATCTGGCCACGTACTATTGTCAGCAGTCCTA
CAGCACCCCTTTCACCTTCGGACCCGGAACAAAGGTGGACATTAAGGCCCTGAGCA
ACAGCATCATGTACTTCAGCCACTTCGTGCCCGTGTTTCTGCCCGCCAAGCCTACAA
CAACCCCTGCTCCTAGACCACCTACACCAGCTCCTACAATCGCCAGCCAGCCTCTGT
CTCTGAGGCCCGAAGCTTGTAGACCAGCTGCTGGCGGAGCCGTGCATACAAGAGGA
CTGGATTTTGCCTGCGACATCTATATCTGGGCCCCTCTGGCTGGCACATGCGGAGTT
CTGCTGCTCAGCCTGGTCATCACCCTGTACTGCAACCACAGAAGAAGCAAGCGGAG
CAGACTGCTGCACAGCGACTACATGAACATGACCCCTAGACGGCCCGGACCTACCA
GAAAGCACTACCAGCCTTACGCTCCTCCTAGAGATTTCGCCGCCTACCGGTCCAGAG
TGAAGTTCAGCAGGAGCGCAGACGCCCCCGCGTACAAGCAGGGCCAGAACCAGCT
CTATAACGAGCTCAATCTAGGACGAAGAGAGGAGTACGATGTTTTGGACAAGAGAC
GTGGCCGGGACCCTGAGATGGGGGGAAAGCCGAGAAGGAAGAACCCTCAGGAAGG
CCTGTACAATGAACTGCAGAAAGATAAGATGGCGGAGGCCTACAGTGAGATTGGG
ATGAAAGGCGAGCGCCGGAGGGGCAAGGGGCACGATGGCCTTTACCAGGGTCTCA
GTACAGCCACCAAGGACACCTACGACGCCCTTCACATGCAGGCCCTGCCCCCTCGC
GGTTCTGGCCAGTGCACAAATTATGCACTGCTGAAGCTCGCCGGGGATGTCGAGAG
TAACCCAGGACCTGGAAGCGGAGAAGGTCGTGGTAGTCTACTAACGTGTGGTGATG
TAGAAGAAAATCCTGGACCTATGGCTCTGCCCGTGACAGCTTTGCTGCTGCCTTTGG
CACTGCTGCTGCATGCTGCTAGACCAGAGGTGCAGCTGGTTGAATCTGGCGGAGGA
CTGGTTCAGCCTGGCGGATCTCTGAGACTGTCTTGTGCCGCCAGCGGCTTCACCTTC
AGCAGATACGATATGCACTGGGTCCGACAGGCCCCTGGCAAAGGACTTGAATGGGT
GTCCGTGATCTGGGGCAACGGCAACACACACTACCACAGCGCCCTGAAGTCCCGGT
TCACCATCTCCAGAGACAACAGCAAGAACACCCTGTACCTGCAGATGAACAGCCTG
AGAGCCGAGGACACCGCCGTGTACTACTGCACCCTGAGAATCAAGGATTGGGGCCA
GGGCACCATGGTCACCGTTTCTTCTGGAGGCGGAGGATCTGGTGGCGGAGGAAGTG
GCGGAGGCGGTTCTGACGTGGTCATGACACAGAGCCCTCTGAGCCTGCCTGTGACA
CTGGGACAGCCTGCCAGCATCAGCTGCAAGTCTAGCCAGTCTCTGGTGGCCAGCGA
CGAGAACACCTACCTGAACTGGTTCCAGCAGAGGCCCGGACAGTCTCCTAGACGGC
TGATCTACCAGGTGTCCAAGCTGGATAGCGGCGTGCCCGATAGATTTTCTGGCAGC
GGCTCTGGCACCGACTTCACCCTGAAGATCAGCAGAGTGGAAGCCGAGGACGTGGG
CGTGTACTACTGTCTGCAAGGCATCCATCTGCCTTGGACCTTTGGCCAGGGCACAAA
GCTGGAAATCAAGAATGGCGCTGCACACCCATCCGATCCTCTCGAGCTGGTGGTTTC
TGGACCTTCTGGCGGCCCTAGCAGCCCTACAACAGGACCTACAAGCACAAGCGGCC
CTGAGGACCAACCTCTGACACCAACAGGCAGCGATCCTCAGTCTGGACTGGGGAGA
CATCTGGGCGTTGTGATCGGCATTCTGGTCGCCGTGATCCTGCTCCTGTTGCTCCTGC
TGCTTCTGTTCCTGATCCTGCGGCACAGAAGGCAGGGCAAGCACTGGACAAGCACC
CAGAGAAAGGCCGACTTTCAGCATCCTGCTGGCGCCGTTGGACCTGAGCCTACAGA
TAGAGGACTGCAGTGGCGGTCTAGCCCTGCCGCTGATGCCCAAGAGGAAAATCTTT
ACGCCGCCGTGAAGCACACCCAGCCTGAGGATGGCGTGGAAATGGACACCAGATCT
CCCCACGATGAGGACCCTCAGGCCGTGACATACGCAGAAGTGAAGCACTCCAGACC
TCGGAGAGAGATGGCAAGCCCTCCATCTCCTCTGAGCGGCGAGTTCCTGGACACCA
AAGACAGACAGGCCGAAGAGGACAGACAGATGGATACCGAAGCCGCCGCTTCTGA
AGCCCCACAGGATGTGACATATGCCCAGCTGCATAGCCTGACACTGCGGAGAGAAG
CCACAGAGCCTCCACCTTCTCAAGAAGGCCCATCTCCTGCCGTGCCTTCCATCTATG
CCACTCTGGCCATTCACtaaTgAggatccggattagtccaatttgttaaagacaggatgggctgcaggaattccgataatc
aacctctggattacaaaatttgtgaaagattgactggtattcttaactatgttgctccttttacgctatgtggatacgctgctttaatgcctttgtatc
atgctattgcttcccgtatggctttcattttctcctccttgtataaatcctggttgctgtctctttatgaggagttgtggcccgttgtcaggcaacgtg
gcgtggtgtgcactgtgtttgctgacgcaacccccactggttggggcattgccaccacctgtcagctcctttccgggactttcgctttccccct
ccctattgccacggcggaactcatcgccgcctgccttgcccgctgctggacaggggctcggctgttgggcactgacaattccgtggtgttg
tcggggaagctgacgtcctttccatggctgctcgcctgtgttgccacctggattctgcgcgggacgtccttctgctacgtcccttcggccctc
aatccagcggaccttccttcccgcggcctgctgccggctctgcggcctcttccgcgtcttcgccttcgccctcagacgagtcggatctccctt
tgggccgcctccccgcctggagaattcgatatcagtggtccaggctctagttttgactcaacaatatcaccagctgaagcctatagagtacg
agccatagataaaataaaagattttatttagtctccagaaaaaggggggaatgaaagaccccacctgtaggtttggcaagctagcaataaaa
gagcccacaacccctcacteggggcgccagtcctccgattgactgagtcgcccggccgcttcgagcagacatgataagatacattgatga
gtttggacaaaccacaactagaatgcagtgaaaaaaatgctttatttgtgaaatttgtgatgctattgctttatttgtaaccattataagctgcaata
aacaagttaacaacaacaattgcattcattttatgtttcaggttcagggggagatgtgggaggttttttaaagcaagtaaaacctctacaaatgt
ggtaaaatcgataaggatcgggtacccgtgtatccaataaaccctcttgcagttgcatccgacttgtggtctcgctgttccttgggagggtctc
ctctgagtgattgactacccgtcagcgggggtctttcacacatgcagcatgtatcaaaattaatttggttttttttcttaagctgtgccttctagttg
ccagccatctgttgtttgcccctcccccgtgccttccttgaccctggaaggtgccactcccactgtcctttcctaataaaatgaggaaattgcat
cgcattgtctgagtaggtgtcattctattctggggggtggggggggcaggacagcaagggggaggattgggaagacaatagcaggcat
gctggggatgcggtgggctctatggagatcccgcggtacctcgcgaatgcatctagatccaatggcctttttggcccagacatgataagata
cattgatgagtttggacaaaccacaactagaatgcagtgaaaaaaatgctttatttgtgaaatttgtgatgctattgctttatttgtaaccattataa
gctgcaataaacaagttgcggccgcttagccctcccacacataaccagagggcagcaattcacgaatcccaactgccgtcggctgtccatc
actgtccttcactatggctttgatcccaggatgcagatcgagaagcacctgtcggcaccgtccgcaggggctcaagatgcccctgttctcatt
tccgatcgcgacgatacaagtcaggttgccagctgccgcagcagcagcagtgcccagcaccacgagttctgcacaaggtcccccagtaa
aatgatatacattgacaccagtgaagatgcggccgtcgctagagagagctgcgctggcgacgctgtagtcttcagagatggggatgctgtt
gattgtagccgttgctctttcaatgagggtggattcttcttgagacaaaggcttggccatgcggccgccgctcggtgttcgaggccacacgc
gtcaccttaatatgcgaagtggacctcggaccgcgccgccccgactgcatctgcgtgttcgaattcgccaatgacaagacgctgggcggg
gtttgtgtcatcatagaactaaagacatgcaaatatatttcttccggggggtaccggcctttttggccATTGGatcggatctggccaaaaa
ggcccttaagtatttacattaaatggccatagtacttaaagttacattggcttccttgaaataaacatggagtattcagaatgtgtcataaatatttctaat
tttaagatagtatctccattggctttctactttttttttatttttttttgtcctctgtcttccatttgttgttgttgttgtttgtttgtttgtttgttggtt
ggttggttaatttttttttaaagatcctacactatagttcaagctagactattagctactctgtaacccagggtgaccttgaagtcatgggtagcctgct
gttttagccttcccacatctaagattacaggtatgagctatcatttttggtatattgattgattgattgattgatgtgtgtgtgtgtgattgtgtttgtgt
gtgtgaTtgtgTaTatgtgtgtatggTtgtgtgtgaTtgtgtgtatgtatgTTtgtgtgtgaTtgTgtgtgtgtgaTtgtgcatgtgtgtgt
gtgtgaTtgtgtTtatgtgtatgaTtgtgtgtgtgtgtgtgtgtgtgtgtgtgtgtgtgtgtgtgtgtgtgttgtgTaTaTatatttatggtagtg
agagGcaacgctccggctcaggtgtcaggttggtttttgagacagagtctttcacttagcttggaattcactggccgtcgttttacaacgtcgt
gactgggaaaaccctggcgttacccaacttaatcgccttgcagcacatccccctttcgccagctggcgtaatagcgaagaggcccgcacc
gatcgcccttcccaacagttgcgcagcctgaatggcgaatggcgcctgatgcggtattttctccttacgcatctgtgcggtatttcacaccgc
atatggtgcactctcagtacaatctgctctgatgccgcatagttaagccagccccgacacccgccaacacccgctgacgcgccctgacgg
gcttgtctgctcccggcatccgcttacagacaagctgtgaccgtctccgggagctgcatgtgtcagaggttttcaccgtcatcaccgaaacg
cgcgagacgaaagggcctcgtgatacgcctatttttataggttaatgtcatgataataatggtttcttagacgtcaggtggcacttttcgggga
aatgtgcgcggaacccctatttgtttatttttctaaatacattcaaatatgtatccgctcatgagacaataaccctgataaatgcttcaataatattg
aaaaaggaagagtatgagtattcaacatttccgtgtcgcccttattcccttttttgcggcattttgccttcctgtttttgctcacccagaaacgctg
gtgaaagtaaaagatgctgaagatcagttgggtgcacgagtgggttacatcgaactggatctcaacagcggtaagatccttgagagttttcg
ccccgaagaacgttttccaatgatgagcacttttaaagttctgctatgtggcgcggtattatcccgtattgacgccgggcaagagcaactcgg
tcgccgcatacactattctcagaatgacttggttgagtactcaccagtcacagaaaagcatcttacggatggcatgacagtaagagaattatg
cagtgctgccataaccatgagtgataacactgcggccaacttacttctgacaacgatcggaggaccgaaggagctaaccgcttttttgcaca
acatgggggatcatgtaactcgccttgatcgttgggaaccggagctgaatgaagccataccaaacgacgagcgtgacaccacgatgcctg
tagcaatggcaacaacgttgcgcaaactattaactggcgaactacttactctagcttcccggcaacaattaatagactggatggaggcggat
aaagttgcaggaccacttctgcgctcggcccttccggctggctggtttattgctgataaatctggagccggtgagcgtgggtctcgcggtat
cattgcagcactggggccagatggtaagccctcccgtatcgtagttatctacacgacggggagtcaggcaactatggatgaacgaaatag
acagatcgctgagataggtgcctcactgattaagcattggtaactgtcagaccaagtttactcatatatactttagattgatttaaaacttcattttt
aatttaaaaggatctaggtgaagatcctttttgataatctcatgaccaaaatcccttaacgtgagttttcgttccactgagcgtcagaccccgtag
aaaagatcaaaggatcttcttgagatcctttttttctgcgcgtaatctgctgcttgcaaacaaaaaaaccaccgctaccagcggtggtttgtttg
ccggatcaagagctaccaactctttttccgaaggtaactggcttcagcagagcgcagataccaaatactgtccttctagtgtagccgtagtta
ggccaccacttcaagaactctgtagcaccgcctacatacctcgctctgctaatcctgttaccagtggctgctgccagtggcgataagtcgtgt
cttaccgggttggactcaagacgatagttaccggataaggcgcagcggtcgggctgaacggggggttcgtgcacacagcccagcttgga
gcgaacgacctacaccgaactgagatacctacagcgtgagctatgagaaagcgccacgcttcccgaagggagaaaggcggacaggtat
ccggtaagcggcagggtcggaacaggagagcgcacgagggagcttccagggggaaacgcctggtatctttatagtcctgtcgggtttcg
ccacctctgacttgagcgtcgatttttgtgatgctcgtcaggggggggagcctatggaaaaacgccagcaacgcggcctttttacggttcc
tggccttttgctggccttttgctcacatgttctttcctgcgttatcccctgattctgtggataaccgtattaccgcctttgagtgagctgataccgct
cgccgcagccgaacgaccgagcgcagcgagtcagtgagcgaggaagcggaagagcgcccaatacgcaaaccgcctctccccgcgc
gttggccgattcattaatgcagctggcacgacaggtttcccgactggaaagcgggcagtgagcgcaacgcaattaatgtgagttagctcac
tcattaggcaccccaggctttacactttatgcttccggctcgtatgttgtgtggaattgtgagcggataacaatttcacacaggaaacagctatg
accatgattacgcc
SB07401; SINvec SV40 (SEQ ID NO: 427)
aagcttgaattcgagcttgcatgcctgcaggtcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggata
gcggtttgactcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagagctcaataaaagagcccaca
acccctcactcggcgcgccagtcctccgattgactgagtcgcccgggtacccgtgtatccaataaaccctcttgcagttgcatccgacttgtg
gtctcgctgttccttgggagggtctcctctgagtgattgactacccgtcagcgggggtctttcatttgggggctcgtccgagatcgggagacc
cctgcccagggaccaccgacccaccaccgggaggtaagctggccagcaacttatctgtgtctgtccgattgtctagtgtctatgactgatttt
atgcgcctgcgtcggtactagttagctaactagctctgtatctggcggacccgtggtggaactgacgagttcggaacacccggccgcaacc
ctgggagacgtcccagggacttcgggggccgtttttgtggcccgacctgagtcctaaaatcccgatcgtttaggactctttggtgcaccccc
cttagaggagggatatgtggttctggtaggagacgagaacctaaaacagttcccgcctccgtctgaatttttgctttcggtttgggaccgaag
ccgcgccgcgcgtcttgtctgctgcagcatcgttctgtgttgtctctgtctgactgtgtttctgtatttgtctgaaaatatgggccccccctcgag
CTGTGGAATGTGTGTCAGTTAGGGTGTGGAAAGTCCCCAGGCTCCCCAGCAGGCAG
AAGTATGCAAAGCATGCATCTCAATTAGTCAGCAACCAGGTGTGGAAAGTCCCCAG
GCTCCCCAGCAGGCAGAAGTATGCAAAGCATGCATCTCAATTAGTCAGCAACCATA
GTCCCGCCCCTAACTCCGCCCATCCCGCCCCTAACTCCGCCCAGTTCCGCCCATTCT
CCGCCCCATGGCTGACTAATTTTTTTTATTTATGCAGAGGCCGAGGCCGCCTCTGCC
TCTGAGCTATTCCAGAAGTAGTGAGGAGGCTTTTTTGGAGGCCTAGGCTTTTGCAAA
AAGCTGGATCCGCGTCAAGTGGAGCAAGGCAGGTGGACAGTCTCGAggccgccaccATG
GACTGGACTTGGATACTCTTTCTGGTCGCTGCCGCCACACGGGTGCACTCTAATTGG
GTCAACGTGATCAGCGACCTGAAGAAGATCGAGGACCTGATCCAGAGCATGCACAT
CGACGCCACACTGTACACCGAGTCCGATGTGCACCCTAGCTGCAAAGTGACCGCCA
TGAAGTGCTTTCTGCTGGAACTGCAAGTGATCAGCCTGGAAAGCGGCGACGCCAGC
ATCCACGATACCGTGGAAAATCTGATCATCCTGGCCAACAACAGCCTGTCCAGCAA
CGGCAATGTGACCGAGAGCGGCTGCAAAGAGTGCGAGGAACTGGAAGAGAAGAAC
ATCAAAGAGTTTCTGCAGAGCTTCGTCCACATCGTGCAGATGTTCATCAACACCTCA
TCAGGCGGCGGTGGTAGTGGAGGCGGAGGCTCAGGCGTGACCCCTGAGCCTATCTT
CAGCCTGATCGGCGGAGGTTCCGGAGGTGGCGGTTCCGGCGGAGGATCTCTTCAAT
TGCTGCCTAGCTGGGCCATCACACTGATCTCCGTGAACGGCATCTTCGTGATCTGCT
GCCTGACCTACTGCTTCGCCCCTAGATGCAGAGAGCGGAGAAGAAACGAGCGGCTG
AGAAGAGAAAGCGTGCGGCCTGTGGGTAGCGGCCAGTGTACCAACTACGCCCTGCT
GAAACTGGCCGGCGACGTGGAATCTAATCCTGGACCTGGATCTGGCGAGGGACGCG
GGAGTCTACTGACGTGTGGAGACGTGGAGGAAAACCCTGGACCTATGCTGCTGCTG
GTTACATCTCTGCTGCTGTGCGAGCTGCCCCATCCTGCCTTTCTGCTTATTCCTGAAG
TGCAGCTGGTTCAGTCTGGCGCCGAAGTGAAGAAACCTGGCAGCAGCGTGAAGGTG
TCCTGCAAAGCTTCTGGCGGCACCTTCAGCAGCTACGCCATCTCTTGGGTTCGACAG
GCCCCTGGACAAGGCCTGGAATGGATGGGAGGCATCATCCCCATCTTCGGCACCGC
CAATTACGCCCAGAAATTCCAGGGCAGAGTGACCATCACCGCCGACAAGAGCACAA
GCACCGCCTACATGGAACTGAGCAGCCTGAGAAGCGAGGACACCGCCGTGTACTAC
TGCGCCACATTTGCCCTGTTCGGCTTCAGAGAGCAGGCCTTCGATATCTGGGGCCAG
GGCACAACCGTGACCGTTTCTAGCGGAGGCGGAGGATCTGGTGGTGGTGGATCTCA
GGTTCAGCTGGTGCAGAGCGGAGCTGAAGTGAAAAAGCCAGGCTCCTCCGTGAAAG
TCTCTTGCAAGGCCAGCGGCTACACCTTTACCGACTACAACATGCACTGGGTCCGAC
AAGCTCCAGGCCAGGGACTTGAGTGGATCGGCTACATCTACCCCTACAACGGCGGC
ACCGGCTACAACCAGAAGTTCAAGAGCAAGGCCACAATCACAGCCGACGAGTCCA
CCAACACAGCTTATATGGAACTGTCTAGCCTGCGCTCCGAGGATACAGCTGTGTACT
ATTGTGCCAGGGGCAGACCCGCCATGGACTATTGGGGACAGGGAACCCTGGTCACA
GTGTCATCTGGTGGCGGAGGAAGTGGCGGCGGAGGCTCTGATATTCAGATGACTCA
GAGCCCCAGCAGCCTGTCTGCCTCTGTGGGAGACAGAGTGACAATTACCTGCCGGG
CCAGCGAGAGCGTGGACAATTACGGCATCAGCTTCATGAACTGGTTCCAGCAGAAG
CCCGGCAAGGCCCCTAAGCTGCTGATCTACGCCGCCAGCAATCAAGGCAGCGGAGT
GCCTAGCAGATTTTCCGGCTCTGGCAGCGGCACCGATTTCACCCTGACCATCAGTAG
CCTGCAGCCTGACGACTTCGCCACCTACTACTGCCAGCAGAGCAAAGAGGTGCCCT
GGACCTTTGGACAGGGCACCAAGGTGGAAATCAAAGGCGGTGGCGGATCTGGCGG
AGGTGGCAGTGATATCCAAATGACCCAGTCTCCATCCAGCCTGAGCGCCAGCGTTG
GCGATAGAGTCACAATCACCTGTAGAGCCAGCCAGAGCATCAGCTCCTACCTGAAT
TGGTATCAGCAGAAACCAGGCAAAGCTCCCAAACTCCTGATCTATGCTGCCTCCAG
CCTGCAGAGTGGCGTGCCATCTAGATTCAGCGGCAGCGGAAGCGGCACAGACTTTA
CACTGACAATATCTTCCCTGCAGCCAGAGGACCTGGCCACATATTATTGCCAGCAGT
CCTACAGCACCCCTTTCACCTTCGGACCCGGAACAAAGGTGGACATTAAGGCCCTG
AGCAACAGCATCATGTACTTCAGCCACTTCGTGCCCGTGTTTCTGCCCGCCAAGCCT
ACAACAACCCCTGCTCCTAGACCACCTACACCAGCTCCTACAATCGCCAGCCAGCCT
CTGTCTCTGAGGCCCGAAGCTTGTAGACCAGCTGCTGGCGGAGCCGTGCATACAAG
AGGACTGGATTTTGCCTGCGACATCTATATCTGGGCCCCTCTGGCTGGCACATGCGG
AGTTCTGCTGCTCAGCCTGGTCATCACCCTGTACTGCAACCACAGAAGAAGCAAGC
GGAGCAGACTGCTGCACAGCGACTACATGAACATGACCCCTAGACGGCCCGGACCT
ACCAGAAAGCACTACCAGCCTTACGCTCCTCCTAGAGATTTCGCCGCCTACCGGTCC
AGAGTGAAGTTCAGCAGGAGCGCAGACGCCCCCGCGTACAAGCAGGGCCAGAACC
AGCTCTATAACGAGCTCAATCTAGGACGAAGAGAGGAGTACGATGTTTTGGACAAG
AGACGTGGCCGGGACCCTGAGATGGGGGGAAAGCCGAGAAGGAAGAACCCTCAGG
AAGGCCTGTACAATGAACTGCAGAAAGATAAGATGGCGGAGGCCTACAGTGAGAT
TGGGATGAAAGGCGAGCGCCGGAGGGGCAAGGGGCACGATGGCCTTTACCAGGGT
CTCAGTACAGCCACCAAGGACACCTACGACGCCCTTCACATGCAGGCCCTGCCCCC
TCGCGGTTCTGGCCAGTGCACAAATTATGCACTGCTGAAGCTCGCCGGGGATGTCG
AGAGTAACCCAGGACCTGGAAGCGGAGAAGGTCGTGGTAGTCTACTAACGTGTGGT
GATGTAGAAGAAAATCCTGGACCTATGGCTCTGCCCGTGACAGCTTTGCTGCTGCCT
TTGGCACTGCTGCTGCATGCTGCTAGACCAGAGGTGCAGCTGGTTGAATCTGGCGG
AGGACTGGTTCAGCCTGGCGGATCTCTGAGACTGTCTTGTGCCGCCAGCGGCTTCAC
CTTCAGCAGATACGATATGCACTGGGTCCGACAGGCCCCTGGCAAAGGACTTGAAT
GGGTGTCCGTGATCTGGGGCAACGGCAACACACACTACCACAGCGCCCTGAAGTCC
CGGTTCACCATCTCCAGAGACAACAGCAAGAACACCCTGTACCTGCAGATGAACAG
CCTGAGAGCCGAGGACACCGCCGTGTACTACTGCACCCTGAGAATCAAGGATTGGG
GCCAGGGCACCATGGTCACCGTTTCTTCTGGAGGCGGAGGATCTGGTGGCGGAGGA
AGTGGCGGAGGCGGTTCTGACGTGGTCATGACACAGAGCCCTCTGAGCCTGCCTGT
GACACTGGGACAGCCTGCCAGCATCAGCTGCAAGTCTAGCCAGTCTCTGGTGGCCA
GCGACGAGAACACCTACCTGAACTGGTTCCAGCAGAGGCCCGGACAGTCTCCTAGA
CGGCTGATCTACCAGGTGTCCAAGCTGGATAGCGGCGTGCCCGATAGATTTTCTGGC
AGCGGCTCTGGCACCGACTTCACCCTGAAGATCAGCAGAGTGGAAGCCGAGGACGT
GGGCGTGTACTACTGTCTGCAAGGCATCCATCTGCCTTGGACCTTTGGCCAGGGCAC
AAAGCTGGAAATCAAGAATGGCGCTGCAACAACAACACCCGCACCTCGGCCTCCAA
CTCCAGCTCCAACAATTGCACTGCAACCCCTGAGTCTGAGGCCCGAGGCCTGTAGG
CCAGCAGCTGGCGGAGCTGTTCACACTAGAGGCCTGGACTTTGCCTGTGACGTGAT
CGGCATTCTGGTCGCCGTGATCCTGCTCCTGTTGCTCCTGCTGCTTCTGTTCCTGATC
CTGCGGCACAGAAGGCAGGGCAAGCACTGGACAAGCACCCAGAGAAAGGCCGACT
TTCAGCATCCTGCTGGCGCCGTTGGACCTGAGCCTACAGATAGAGGACTGCAGTGG
CGGTCTAGCCCTGCCGCTGATGCCCAAGAGGAAAATCTTTACGCCGCCGTGAAGCA
CACCCAGCCTGAGGATGGCGTGGAAATGGACACCAGATCTCCCCACGATGAGGACC
CTCAGGCCGTGACATACGCAGAAGTGAAGCACTCCAGACCTCGGAGAGAGATGGC
AAGCCCTCCATCTCCTCTGAGCGGCGAGTTCCTGGACACCAAAGACAGACAGGCCG
AAGAGGACAGACAGATGGATACCGAAGCCGCCGCTTCTGAAGCCCCACAGGATGT
GACATATGCCCAGCTGCATAGCCTGACACTGCGGAGAGAAGCCACAGAGCCTCCAC
CTTCTCAAGAAGGCCCATCTCCTGCCGTGCCTTCCATCTATGCCACTCTGGCCATTC
ACtaaTgAggatccggattagtccaatttgttaaagacaggatgggctgcaggaattccgataatcaacctctggattacaaaatttgtga
aagattgactggtattcttaactatgttgctccttttacgctatgtggatacgctgctttaatgcctttgtatcatgctattgcttcccgtatggctttc
attttctcctccttgtataaatcctggttgctgtctctttatgaggagttgtggcccgttgtcaggcaacgtggcgtggtgtgcactgtgtttgctg
acgcaacccccactggttggggcattgccaccacctgtcagctcctttccgggactttcgctttccccctccctattgccacggcggaactca
tcgccgcctgccttgcccgctgctggacaggggctcggctgttgggcactgacaattccgtggtgttgtcggggaagctgacgtcctttcca
tggctgctcgcctgtgttgccacctggattctgcgcgggacgtccttctgctacgtcccttcggccctcaatccagcggaccttccttcccgc
ggcctgctgccggctctgcggcctcttccgcgtcttcgccttcgccctcagacgagtcggatctccctttgggccgcctccccgcctggaga
attcgatatcagtggtccaggctctagttttgactcaacaatatcaccagctgaagcctatagagtacgagccatagataaaataaaagatttta
tttagtctccagaaaaaggggggaatgaaagaccccacctgtaggtttggcaagctagcaataaaagagcccacaacccctcactcgggg
cgccagtcctccgattgactgagtcgcccggccgcttcgagcagacatgataagatacattgatgagtttggacaaaccacaactagaatg
cagtgaaaaaaatgctttatttgtgaaatttgtgatgctattgctttatttgtaaccattataagctgcaataaacaagttaacaacaacaattgcat
tcattttatgtttcaggttcagggggagatgtgggaggttttttaaagcaagtaaaacctctacaaatgtggtaaaatcgataaggatcgggta
cccgtgtatccaataaaccctcttgcagttgcatccgacttgtggtctcgctgttccttggggggtctcctctgagtgattgactacccgtcag
cgggggtctttcacacatgcagcatgtatcaaaattaatttggttttttttcttaagctgtgccttctagttgccagccatctgttgtttgcccctccc
ccgtgccttccttgaccctggaaggtgccactcccactgtcctttcctaataaaatgaggaaattgcatcgcattgtctgagtaggtgtcattct
attctggggggtggggggggcaggacagcaagggggaggattgggaagacaatagcaggcatgctggggatgcggtgggctctatg
gagatcccgcggtacctcgcgaatgcatctagatccaatggcctttttggcccagacatgataagatacattgatgagtttggacaaaccaca
actagaatgcagtgaaaaaaatgctttatttgtgaaatttgtgatgctattgctttatttgtaaccattataagctgcaataaacaagttgcggccg
cttagccctcccacacataaccagagggcagcaattcacgaatcccaactgccgtcggctgtccatcactgtccttcactatggctttgatcc
caggatgcagatcgagaagcacctgtcggcaccgtccgcaggggctcaagatgcccctgttctcatttccgatcgcgacgatacaagtca
ggttgccagctgccgcagcagcagcagtgcccagcaccacgagttctgcacaaggtcccccagtaaaatgatatacattgacaccagtga
agatgcggccgtcgctagagagagctgcgctggcgacgctgtagtcttcagagatggggatgctgttgattgtagccgttgctctttcaatg
agggtggattcttcttgagacaaaggcttggccatgcggccgccgctcggtgttcgaggccacacgcgtcaccttaatatgcgaagtggac
ctcggaccgcgccgccccgactgcatctgcgtgttcgaattcgccaatgacaagacgctgggcggggtttgtgtcatcatagaactaaaga
catgcaaatatatttcttccggggggtaccggcctttttggccATTGGatcggatctggccaaaaaggcccttaagtatttacattaaatg
gccatagtacttaaagttacattggcttccttgaaataaacatggagtattcagaatgtgtcataaatatttctaattttaagatagtatctccattggctt
tctactttttcttttatttttttttgtcctctgtcttccatttgttgttgttgttgtttgtttgtttgtttgttggttggttggttaatttttttttaaag
atcctacactatagttcaagctagactattagctactctgtaacccagggtgaccttgaagtcatgggtagcctgctgttttagccttcccacatctaaga
ttacaggtatgagctatcatttttggtatattgattgattgattgattgatgtgtgtgtgtgtgattgtgtttgtgtgtgtgaTtgtgTaTatgtgtgt
atggTtgtgtgtgaTtgtgtgtatgtatgTTtgtgtgtgaTtgTgtgtgtgtgaTtgtgcatgtgtgtgtgtgtgaTtgtgtTtatgtgtatg
aTtgtgtgtgtgtgtgtgtgtgtgtgtgtgtgtgtgtgtgtgtgtgtgttgtgTaTaTatatttatggtagtgagagGcaacgctccggctca
ggtgtcaggttggtttttgagacagagtctttcacttagcttggaattcactggccgtcgttttacaacgtcgtgactgggaaaaccctggcgtt
acccaacttaatcgccttgcagcacatccccctttcgccagctggcgtaatagcgaagaggcccgcaccgatcgcccttcccaacagttgc
gcagcctgaatggcgaatggcgcctgatgcggtattttctccttacgcatctgtgcggtatttcacaccgcatatggtgcactctcagtacaat
ctgctctgatgccgcatagttaagccagccccgacacccgccaacacccgctgacgcgccctgacgggcttgtctgctcccggcatccgc
ttacagacaagctgtgaccgtctccgggagctgcatgtgtcagaggttttcaccgtcatcaccgaaacgcgcgagacgaaagggcctcgt
gatacgcctatttttataggttaatgtcatgataataatggtttcttagacgtcaggtggcacttttcggggaaatgtgcgcggaacccctatttgt
ttatttttctaaatacattcaaatatgtatccgctcatgagacaataaccctgataaatgcttcaataatattgaaaaaggaagagtatgagtattc
aacatttccgtgtcgcccttattcccttttttgcggcattttgccttcctgtttttgctcacccagaaacgctggtgaaagtaaaagatgctgaaga
tcagttgggtgcacgagtgggttacatcgaactggatctcaacagcggtaagatccttgagagttttcgccccgaagaacgttttccaatgat
gagcacttttaaagttctgctatgtggcgcggtattatcccgtattgacgccgggcaagagcaactcggtcgccgcatacactattctcagaa
tgacttggttgagtactcaccagtcacagaaaagcatcttacggatggcatgacagtaagagaattatgcagtgctgccataaccatgagtg
ataacactgcggccaacttacttctgacaacgatcggaggaccgaaggagctaaccgcttttttgcacaacatgggggatcatgtaactcgc
cttgatcgttgggaaccggagctgaatgaagccataccaaacgacgagcgtgacaccacgatgcctgtagcaatggcaacaacgttgcg
caaactattaactggcgaactacttactctagcttcccggcaacaattaatagactggatggaggcggataaagttgcaggaccacttctgcg
ctcggcccttccggctggctggtttattgctgataaatctggagccggtgagcgtgggtctcgcggtatcattgcagcactggggccagatg
gtaagccctcccgtatcgtagttatctacacgacggggagtcaggcaactatggatgaacgaaatagacagatcgctgagataggtgcctc
actgattaagcattggtaactgtcagaccaagtttactcatatatactttagattgatttaaaacttcatttttaatttaaaaggatctaggtgaagat
cctttttgataatctcatgaccaaaatcccttaacgtgagttttcgttccactgagcgtcagaccccgtagaaaagatcaaaggatcttcttgag
atcctttttttctgcgcgtaatctgctgcttgcaaacaaaaaaaccaccgctaccagcggtggtttgtttgccggatcaagagctaccaactcttt
ttccgaaggtaactggcttcagcagagcgcagataccaaatactgtccttctagtgtagccgtagttaggccaccacttcaagaactctgtag
caccgcctacatacctcgctctgctaatcctgttaccagtggctgctgccagtggcgataagtcgtgtcttaccgggttggactcaagacgat
agttaccggataaggcgcagcggtcgggctgaacggggggttcgtgcacacagcccagcttggagcgaacgacctacaccgaactgag
atacctacagcgtgagctatgagaaagcgccacgcttcccgaagggagaaaggcggacaggtatccggtaagcggcagggtcggaac
aggagagcgcacgagggagcttccagggggaaacgcctggtatctttatagtcctgtcgggtttcgccacctctgacttgagcgtcgattttt
gtgatgctcgtcaggggggcggagcctatggaaaaacgccagcaacgcggcctttttacggttcctggccttttgctggccttttgctcacat
gttctttcctgcgttatcccctgattctgtggataaccgtattaccgcctttgagtgagctgataccgctcgccgcagccgaacgaccgagcg
cagcgagtcagtgagcgaggaagcggaagagcgcccaatacgcaaaccgcctctccccgcgcgttggccgattcattaatgcagctgg
cacgacaggtttcccgactggaaagcgggcagtgagcgcaacgcaattaatgtgagttagctcactcattaggcaccccaggctttacactt
tatgcttccggctcgtatgttgtgtggaattgtgagcggataacaatttcacacaggaaacagctatgaccatgattacgcc
SB07405; SINvec SV40 (SEQ ID NO: 428)
aagcttgaattcgagcttgcatgcctgcaggtcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggata
gcggtttgactcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagagctcaataaaagagcccaca
acccctcactcggcgcgccagtcctccgattgactgagtcgcccgggtacccgtgtatccaataaaccctcttgcagttgcatccgacttgtg
gtctcgctgttccttgggagggtctcctctgagtgattgactacccgtcagcgggggtctttcatttgggggctcgtccgagatcgggagacc
cctgcccagggaccaccgacccaccaccgggaggtaagctggccagcaacttatctgtgtctgtccgattgtctagtgtctatgactgatttt
atgcgcctgcgtcggtactagttagctaactagctctgtatctggcggacccgtggtggaactgacgagttcggaacacccggccgcaacc
ctgggagacgtcccagggacttcgggggccgtttttgtggcccgacctgagtcctaaaatcccgatcgtttaggactctttggtgcaccccc
cttagaggagggatatgtggttctggtaggagacgagaacctaaaacagttcccgcctccgtctgaatttttgctttcggtttgggaccgaag
ccgcgccgcgcgtcttgtctgctgcagcatcgttctgtgttgtctctgtctgactgtgtttctgtatttgtctgaaaatatgggccccccctcgag
CTGTGGAATGTGTGTCAGTTAGGGTGTGGAAAGTCCCCAGGCTCCCCAGCAGGCAG
AAGTATGCAAAGCATGCATCTCAATTAGTCAGCAACCAGGTGTGGAAAGTCCCCAG
GCTCCCCAGCAGGCAGAAGTATGCAAAGCATGCATCTCAATTAGTCAGCAACCATA
GTCCCGCCCCTAACTCCGCCCATCCCGCCCCTAACTCCGCCCAGTTCCGCCCATTCT
CCGCCCCATGGCTGACTAATTTTTTTTATTTATGCAGAGGCCGAGGCCGCCTCTGCC
TCTGAGCTATTCCAGAAGTAGTGAGGAGGCTTTTTTGGAGGCCTAGGCTTTTGCAAA
AAGCTGGATCCGCGTCAAGTGGAGCAAGGCAGGTGGACAGTCTCGAggccgccaccATG
GACTGGACTTGGATACTCTTTCTGGTCGCTGCCGCCACACGGGTGCACTCTAATTGG
GTCAACGTGATCAGCGACCTGAAGAAGATCGAGGACCTGATCCAGAGCATGCACAT
CGACGCCACACTGTACACCGAGTCCGATGTGCACCCTAGCTGCAAAGTGACCGCCA
TGAAGTGCTTTCTGCTGGAACTGCAAGTGATCAGCCTGGAAAGCGGCGACGCCAGC
ATCCACGATACCGTGGAAAATCTGATCATCCTGGCCAACAACAGCCTGTCCAGCAA
CGGCAATGTGACCGAGAGCGGCTGCAAAGAGTGCGAGGAACTGGAAGAGAAGAAC
ATCAAAGAGTTTCTGCAGAGCTTCGTCCACATCGTGCAGATGTTCATCAACACCTCA
TCAGGCGGCGGTGGTAGTGGAGGCGGAGGCTCAGGCGTGACCCCTGAGCCTATCTT
CAGCCTGATCGGCGGAGGTTCCGGAGGTGGCGGTTCCGGCGGAGGATCTCTTCAAT
TGCTGCCTAGCTGGGCCATCACACTGATCTCCGTGAACGGCATCTTCGTGATCTGCT
GCCTGACCTACTGCTTCGCCCCTAGATGCAGAGAGCGGAGAAGAAACGAGCGGCTG
AGAAGAGAAAGCGTGCGGCCTGTGGGTAGCGGCCAGTGTACCAACTACGCCCTGCT
GAAACTGGCCGGCGACGTGGAATCTAATCCTGGACCTGGATCTGGCGAGGGACGCG
GGAGTCTACTGACGTGTGGAGACGTGGAGGAAAACCCTGGACCTATGCTGCTGCTG
GTTACATCTCTGCTGCTGTGCGAGCTGCCCCATCCTGCCTTTCTGCTTATTCCTGAAG
TGCAGCTGGTTCAGTCTGGCGCCGAAGTGAAGAAACCTGGCAGCAGCGTGAAGGTG
TCCTGCAAAGCTTCTGGCGGCACCTTCAGCAGCTACGCCATCTCTTGGGTTCGACAG
GCCCCTGGACAAGGCCTGGAATGGATGGGAGGCATCATCCCCATCTTCGGCACCGC
CAATTACGCCCAGAAATTCCAGGGCAGAGTGACCATCACCGCCGACAAGAGCACAA
GCACCGCCTACATGGAACTGAGCAGCCTGAGAAGCGAGGACACCGCCGTGTACTAC
TGCGCCACATTTGCCCTGTTCGGCTTCAGAGAGCAGGCCTTCGATATCTGGGGCCAG
GGCACAACCGTGACCGTTTCTAGCGGAGGCGGAGGATCTGGTGGTGGTGGATCTCA
GGTTCAGCTGGTGCAGAGCGGAGCTGAAGTGAAAAAGCCAGGCTCCTCCGTGAAAG
TCTCTTGCAAGGCCAGCGGCTACACCTTTACCGACTACAACATGCACTGGGTCCGAC
AAGCTCCAGGCCAGGGACTTGAGTGGATCGGCTACATCTACCCCTACAACGGCGGC
ACCGGCTACAACCAGAAGTTCAAGAGCAAGGCCACAATCACAGCCGACGAGTCCA
CCAACACAGCTTATATGGAACTGTCTAGCCTGCGCTCCGAGGATACAGCTGTGTACT
ATTGTGCCAGGGGCAGACCCGCCATGGACTATTGGGGACAGGGAACCCTGGTCACA
GTGTCATCTGGTGGCGGAGGAAGTGGCGGCGGAGGCTCTGATATTCAGATGACTCA
GAGCCCCAGCAGCCTGTCTGCCTCTGTGGGAGACAGAGTGACAATTACCTGCCGGG
CCAGCGAGAGCGTGGACAATTACGGCATCAGCTTCATGAACTGGTTCCAGCAGAAG
CCCGGCAAGGCCCCTAAGCTGCTGATCTACGCCGCCAGCAATCAAGGCAGCGGAGT
GCCTAGCAGATTTTCCGGCTCTGGCAGCGGCACCGATTTCACCCTGACCATCAGTAG
CCTGCAGCCTGACGACTTCGCCACCTACTACTGCCAGCAGAGCAAAGAGGTGCCCT
GGACCTTTGGACAGGGCACCAAGGTGGAAATCAAAGGCGGTGGCGGATCTGGCGG
AGGTGGCAGTGATATCCAAATGACCCAGTCTCCATCCAGCCTGAGCGCCAGCGTTG
GCGATAGAGTCACAATCACCTGTAGAGCCAGCCAGAGCATCAGCTCCTACCTGAAT
TGGTATCAGCAGAAACCAGGCAAAGCTCCCAAACTCCTGATCTATGCTGCCTCCAG
CCTGCAGAGTGGCGTGCCATCTAGATTCAGCGGCAGCGGAAGCGGCACAGACTTTA
CACTGACAATATCTTCCCTGCAGCCAGAGGACCTGGCCACATATTATTGCCAGCAGT
CCTACAGCACCCCTTTCACCTTCGGACCCGGAACAAAGGTGGACATTAAGGCCCTG
AGCAACAGCATCATGTACTTCAGCCACTTCGTGCCCGTGTTTCTGCCCGCCAAGCCT
ACAACAACCCCTGCTCCTAGACCACCTACACCAGCTCCTACAATCGCCAGCCAGCCT
CTGTCTCTGAGGCCCGAAGCTTGTAGACCAGCTGCTGGCGGAGCCGTGCATACAAG
AGGACTGGATTTTGCCTGCGACATCTATATCTGGGCCCCTCTGGCTGGCACATGCGG
AGTTCTGCTGCTCAGCCTGGTCATCACCCTGTACTGCAACCACAGAAGAAGCAAGC
GGAGCAGACTGCTGCACAGCGACTACATGAACATGACCCCTAGACGGCCCGGACCT
ACCAGAAAGCACTACCAGCCTTACGCTCCTCCTAGAGATTTCGCCGCCTACCGGTCC
AGAGTGAAGTTCAGCAGGAGCGCAGACGCCCCCGCGTACAAGCAGGGCCAGAACC
AGCTCTATAACGAGCTCAATCTAGGACGAAGAGAGGAGTACGATGTTTTGGACAAG
AGACGTGGCCGGGACCCTGAGATGGGGGGAAAGCCGAGAAGGAAGAACCCTCAGG
AAGGCCTGTACAATGAACTGCAGAAAGATAAGATGGCGGAGGCCTACAGTGAGAT
TGGGATGAAAGGCGAGCGCCGGAGGGGCAAGGGGCACGATGGCCTTTACCAGGGT
CTCAGTACAGCCACCAAGGACACCTACGACGCCCTTCACATGCAGGCCCTGCCCCC
TCGCGGTTCTGGCCAGTGCACAAATTATGCACTGCTGAAGCTCGCCGGGGATGTCG
AGAGTAACCCAGGACCTGGAAGCGGAGAAGGTCGTGGTAGTCTACTAACGTGTGGT
GATGTAGAAGAAAATCCTGGACCTATGGCTCTGCCCGTGACAGCTTTGCTGCTGCCT
TTGGCACTGCTGCTGCATGCTGCTAGACCAGAGGTGCAGCTGGTTGAATCTGGCGG
AGGACTGGTTCAGCCTGGCGGATCTCTGAGACTGTCTTGTGCCGCCAGCGGCTTCAC
CTTCAGCAGATACGATATGCACTGGGTCCGACAGGCCCCTGGCAAAGGACTTGAAT
GGGTGTCCGTGATCTGGGGCAACGGCAACACACACTACCACAGCGCCCTGAAGTCC
CGGTTCACCATCTCCAGAGACAACAGCAAGAACACCCTGTACCTGCAGATGAACAG
CCTGAGAGCCGAGGACACCGCCGTGTACTACTGCACCCTGAGAATCAAGGATTGGG
GCCAGGGCACCATGGTCACCGTTTCTTCTGGAGGCGGAGGATCTGGTGGCGGAGGA
AGTGGCGGAGGCGGTTCTGACGTGGTCATGACACAGAGCCCTCTGAGCCTGCCTGT
GACACTGGGACAGCCTGCCAGCATCAGCTGCAAGTCTAGCCAGTCTCTGGTGGCCA
GCGACGAGAACACCTACCTGAACTGGTTCCAGCAGAGGCCCGGACAGTCTCCTAGA
CGGCTGATCTACCAGGTGTCCAAGCTGGATAGCGGCGTGCCCGATAGATTTTCTGGC
AGCGGCTCTGGCACCGACTTCACCCTGAAGATCAGCAGAGTGGAAGCCGAGGACGT
GGGCGTGTACTACTGTCTGCAAGGCATCCATCTGCCTTGGACCTTTGGCCAGGGCAC
AAAGCTGGAAATCAAGAATGGCGCTGCACACCCATCCGATCCTCTCGAGCTGGTGG
TTTCTGGACCTTCTGGCGGCCCTAGCAGCCCTACAACAGGACCTACAAGCACAAGC
GGCCCTGAGGACCAACCTCTGACACCAACAGGCAGCGATCCTCAGTCTGGACTGGG
GAGACATCTGGGCGTTGTGATCGGCATTCTGGTCGCCGTGATCCTGCTCCTGTTGCT
CCTGCTGCTTCTGTTCCTGATCCTGCGGCACAGAAGGCAGGGCAAGCACTGGACAA
GCACCCAGAGAAAGGCCGACTTTCAGCATCCTGCTGGCGCCGTTGGACCTGAGCCT
ACAGATAGAGGACTGCAGTGGCGGTCTAGCCCTGCCGCTGATGCCCAAGAGGAAAA
TCTTTACGCCGCCGTGAAGCACACCCAGCCTGAGGATGGCGTGGAAATGGACACCA
GATCTCCCCACGATGAGGACCCTCAGGCCGTGACATACGCAGAAGTGAAGCACTCC
AGACCTCGGAGAGAGATGGCAAGCCCTCCATCTCCTCTGAGCGGCGAGTTCCTGGA
CACCAAAGACAGACAGGCCGAAGAGGACAGACAGATGGATACCGAAGCCGCCGCT
TCTGAAGCCCCACAGGATGTGACATATGCCCAGCTGCATAGCCTGACACTGCGGAG
AGAAGCCACAGAGCCTCCACCTTCTCAAGAAGGCCCATCTCCTGCCGTGCCTTCCAT
CTATGCCACTCTGGCCATTCACtaaTgAggatccggattagtccaatttgttaaagacaggatgggctgcaggaattc
cgataatcaacctctggattacaaaatttgtgaaagattgactggtattcttaactatgttgctccttttacgctatgtggatacgctgctttaatgc
ctttgtatcatgctattgcttcccgtatggctttcattttctcctccttgtataaatcctggttgctgtctctttatgaggagttgtggcccgttgtcag
gcaacgtggcgtggtgtgcactgtgtttgctgacgcaacccccactggttggggcattgccaccacctgtcagctcctttccgggactttcg
ctttccccctccctattgccacggcggaactcatcgccgcctgccttgcccgctgctggacaggggctcggctgttgggcactgacaattcc
gtggtgttgtcggggaagctgacgtcctttccatggctgctcgcctgtgttgccacctggattctgcgcgggacgtccttctgctacgtccctt
cggccctcaatccagcggaccttccttcccgcggcctgctgccggctctgcggcctcttccgcgtcttcgccttcgccctcagacgagtcg
gatctccctttgggccgcctccccgcctggagaattcgatatcagtggtccaggctctagttttgactcaacaatatcaccagctgaagcctat
agagtacgagccatagataaaataaaagattttatttagtctccagaaaaaggggggaatgaaagaccccacctgtaggtttggcaagctag
caataaaagagcccacaacccctcactcggggcgccagtcctccgattgactgagtcgcccggccgcttcgagcagacatgataagatac
attgatgagtttggacaaaccacaactagaatgcagtgaaaaaaatgctttatttgtgaaatttgtgatgctattgctttatttgtaaccattataag
ctgcaataaacaagttaacaacaacaattgcattcattttatgtttcaggttcagggggagatgtgggaggttttttaaagcaagtaaaacctct
acaaatgtggtaaaatcgataaggatcgggtacccgtgtatccaataaaccctcttgcagttgcatccgacttgtggtctcgctgttccttggg
agggtctcctctgagtgattgactacccgtcagcgggggtctttcacacatgcagcatgtatcaaaattaatttggttttttttcttaagctgtgcc
ttctagttgccagccatctgttgtttgcccctcccccgtgccttccttgaccctggaaggtgccactcccactgtcctttcctaataaaatgagga
aattgcatcgcattgtctgagtaggtgtcattctattctggggggtggggggggcaggacagcaagggggaggattgggaagacaatag
caggcatgctggggatgcggtgggctctatggagatcccgcggtacctcgcgaatgcatctagatccaatggcctttttggcccagacatg
ataagatacattgatgagtttggacaaaccacaactagaatgcagtgaaaaaaatgctttatttgtgaaatttgtgatgctattgctttatttgtaa
ccattataagctgcaataaacaagttgcggccgcttagccctcccacacataaccagagggcagcaattcacgaatcccaactgccgtcgg
ctgtccatcactgtccttcactatggctttgatcccaggatgcagatcgagaagcacctgtcggcaccgtccgcaggggctcaagatgcccc
tgttctcatttccgatcgcgacgatacaagtcaggttgccagctgccgcagcagcagcagtgcccagcaccacgagttctgcacaaggtcc
cccagtaaaatgatatacattgacaccagtgaagatgcggccgtcgctagagagagctgcgctggcgacgctgtagtcttcagagatggg
gatgctgttgattgtagccgttgctctttcaatgagggtggattcttcttgagacaaaggcttggccatgcggccgccgctcggtgttcgagg
ccacacgcgtcaccttaatatgcgaagtggacctcggaccgcgccgccccgactgcatctgcgtgttcgaattcgccaatgacaagacgct
gggcggggtttgtgtcatcatagaactaaagacatgcaaatatatttcttccggggggtaccggcctttttggccATTGGatcggatctg
gccaaaaaggcccttaagtatttacattaaatggccatagtacttaaagttacattggcttccttgaaataaacatggagtattcagaatgtgtcataaa
tatttctaattttaagatagtatctccattggctttctactttttcttttatttttttttgtcctctgtcttccatttgttgttgttgttgtttgtttgt
ttgtttgttggttggttggttaatttttttttaaagatcctacactatagttcaagctagactattagctactctgtaacccagggtgaccttgaagtcatgg
gtagcctgctgttttagccttcccacatctaagattacaggtatgagctatcatttttggtatattgattgattgattgattgatgtgtgtgtgtgtgatt
gtgtttgtgtgtgtgaTtgtgTaTatgtgtgtatggTtgtgtgtgaTtgtgtgtatgtatgTTtgtgtgtgaTtgTgtgtgtgtgaTtgtgca
tgtgtgtgtgtgtgaTtgtgtTtatgtgtatgaTtgtgtgtgtgtgtgtgtgtgtgtgtgtgtgtgtgtgtgtgtgtgtgttgtgTaTaTatattta
tggtagtgagagGcaacgctccggctcaggtgtcaggttggtttttgagacagagtctttcacttagcttggaattcactggccgtcgttttac
aacgtcgtgactgggaaaaccctggcgttacccaacttaatcgccttgcagcacatccccctttcgccagctggcgtaatagcgaagaggc
ccgcaccgatcgcccttcccaacagttgcgcagcctgaatggcgaatggcgcctgatgcggtattttctccttacgcatctgtgcggtatttc
acaccgcatatggtgcactctcagtacaatctgctctgatgccgcatagttaagccagccccgacacccgccaacacccgctgacgcgcc
ctgacgggcttgtctgctcccggcatccgcttacagacaagctgtgaccgtctccgggagctgcatgtgtcagaggttttcaccgtcatcac
cgaaacgcgcgagacgaaagggcctcgtgatacgcctatttttataggttaatgtcatgataataatggtttcttagacgtcaggtggcactttt
cggggaaatgtgcgcggaacccctatttgtttatttttctaaatacattcaaatatgtatccgctcatgagacaataaccctgataaatgcttcaat
aatattgaaaaaggaagagtatgagtattcaacatttccgtgtcgcccttattcccttttttgcggcattttgccttcctgtttttgctcacccagaa
acgctggtgaaagtaaaagatgctgaagatcagttgggtgcacgagtgggttacatcgaactggatctcaacagcggtaagatccttgaga
gttttcgccccgaagaacgttttccaatgatgagcacttttaaagttctgctatgtggcgcggtattatcccgtattgacgccgggcaagagca
actcggtcgccgcatacactattctcagaatgacttggttgagtactcaccagtcacagaaaagcatcttacggatggcatgacagtaagag
aattatgcagtgctgccataaccatgagtgataacactgcggccaacttacttctgacaacgatcggaggaccgaaggagctaaccgcttttt
tgcacaacatgggggatcatgtaactcgccttgatcgttgggaaccggagctgaatgaagccataccaaacgacgagcgtgacaccacg
atgcctgtagcaatggcaacaacgttgcgcaaactattaactggcgaactacttactctagcttcccggcaacaattaatagactggatggag
gcggataaagttgcaggaccacttctgcgctcggcccttccggctggctggtttattgctgataaatctggagccggtgagcgtgggtctcg
cggtatcattgcagcactggggccagatggtaagccccccgtatcgtagttatctacacgacggggagtcaggcaactatggatgaacga
aatagacagatcgctgagataggtgcctcactgattaagcattggtaactgtcagaccaagtttactcatatatactttagattgatttaaaactt
catttttaatttaaaaggatctaggtgaagatcctttttgataatctcatgaccaaaatcccttaacgtgagttttcgttccactgagcgtcagacc
ccgtagaaaagatcaaaggatcttcttgagatcctttttttctgcgcgtaatctgctgcttgcaaacaaaaaaaccaccgctaccagcggtggt
ttgtttgccggatcaagagctaccaactctttttccgaaggtaactggcttcagcagagcgcagataccaaatactgtccttctagtgtagccg
tagttaggccaccacttcaagaactctgtagcaccgcctacatacctcgctctgctaatcctgttaccagtggctgctgccagtggcgataag
tcgtgtcttaccgggttggactcaagacgatagttaccggataaggcgcagcggtcgggctgaacggggggttcgtgcacacagcccag
cttggagcgaacgacctacaccgaactgagatacctacagcgtgagctatgagaaagcgccacgcttcccgaagggagaaaggcggac
aggtatccggtaagcggcagggtcggaacaggagagcgcacgagggagcttccagggggaaacgcctggtatctttatagtcctgtcgg
gtttcgccacctctgacttgagcgtcgatttttgtgatgctcgtcaggggggcggagcctatggaaaaacgccagcaacgcggcctttttac
ggttcctggccttttgctggccttttgctcacatgttctttcctgcgttatcccctgattctgtggataaccgtattaccgcctttgagtgagctgat
accgctcgccgcagccgaacgaccgagcgcagcgagtcagtgagcgaggaagcggaagagcgcccaatacgcaaaccgcctctccc
cgcgcgttggccgattcattaatgcagctggcacgacaggtttcccgactggaaagcgggcagtgagcgcaacgcaattaatgtgagtta
gctcactcattaggcaccccaggctttacactttatgcttccggctcgtatgttgtgtggaattgtgagcggataacaatttcacacaggaaac
agctatgaccatgattacgcc
SB07412; SINvec SV40 (SEQ ID NO: 429)
aagcttgaattcgagcttgcatgcctgcaggtcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggata
gcggtttgactcacggggatttccaagtctccaccccattgacgtcaatgggagtttgttttggcaccaaaatcaacgggactttccaaaatgt
cgtaacaactccgccccattgacgcaaatgggggtaggcgtgtacggtgggaggtctatataagcagagctcaataaaagagcccaca
acccctcactcggcgcgccagtcctccgattgactgagtcgcccgggtacccgtgtatccaataaaccctcttgcagttgcatccgacttgtg
gtctcgctgttccttgggagggtctcctctgagtgattgactacccgtcagcgggggtctttcatttgggggctcgtccgagatcgggagacc
cctgcccagggaccaccgacccaccaccgggaggtaagctggccagcaacttatctgtgtctgtccgattgtctagtgtctatgactgatttt
atgcgcctgcgtcggtactagttagctaactagctctgtatctggcggacccgtggtggaactgacgagttcggaacacccggccgcaacc
ctgggagacgtcccagggacttcgggggccgtttttgtggcccgacctgagtcctaaaatcccgatcgtttaggactctttggtgcaccccc
cttagaggagggatatgtggttctggtaggagacgagaacctaaaacagttcccgcctccgtctgaatttttgctttcggtttgggaccgaag
ccgcgccgcgcgtcttgtctgctgcagcatcgttctgtgttgtctctgtctgactgtgtttctgtatttgtctgaaaatatgggccccccctcgag
CTGTGGAATGTGTGTCAGTTAGGGTGTGGAAAGTCCCCAGGCTCCCCAGCAGGCAG
AAGTATGCAAAGCATGCATCTCAATTAGTCAGCAACCAGGTGTGGAAAGTCCCCAG
GCTCCCCAGCAGGCAGAAGTATGCAAAGCATGCATCTCAATTAGTCAGCAACCATA
GTCCCGCCCCTAACTCCGCCCATCCCGCCCCTAACTCCGCCCAGTTCCGCCCATTCT
CCGCCCCATGGCTGACTAATTTTTTTTATTTATGCAGAGGCCGAGGCCGCCTCTGCC
TCTGAGCTATTCCAGAAGTAGTGAGGAGGCTTTTTTGGAGGCCTAGGCTTTTGCAAA
AAGCTGGATCCGCGTCAAGTGGAGCAAGGCAGGTGGACAGTCTCGAggccgccaccATG
GACTGGACTTGGATACTCTTTCTGGTCGCTGCCGCCACACGGGTGCACTCTAATTGG
GTCAACGTGATCAGCGACCTGAAGAAGATCGAGGACCTGATCCAGAGCATGCACAT
CGACGCCACACTGTACACCGAGTCCGATGTGCACCCTAGCTGCAAAGTGACCGCCA
TGAAGTGCTTTCTGCTGGAACTGCAAGTGATCAGCCTGGAAAGCGGCGACGCCAGC
ATCCACGATACCGTGGAAAATCTGATCATCCTGGCCAACAACAGCCTGTCCAGCAA
CGGCAATGTGACCGAGAGCGGCTGCAAAGAGTGCGAGGAACTGGAAGAGAAGAAC
ATCAAAGAGTTTCTGCAGAGCTTCGTCCACATCGTGCAGATGTTCATCAACACCTCA
TCAGGCGGCGGTGGTAGTGGAGGCGGAGGCTCAGGCGTGACCCCTGAGCCTATCTT
CAGCCTGATCGGCGGAGGTTCCGGAGGTGGCGGTTCCGGCGGAGGATCTCTTCAAT
TGCTGCCTAGCTGGGCCATCACACTGATCTCCGTGAACGGCATCTTCGTGATCTGCT
GCCTGACCTACTGCTTCGCCCCTAGATGCAGAGAGCGGAGAAGAAACGAGCGGCTG
AGAAGAGAAAGCGTGCGGCCTGTGGGTAGCGGCCAGTGTACCAACTACGCCCTGCT
GAAACTGGCCGGCGACGTGGAATCTAATCCTGGACCTGGATCTGGCGAGGGACGCG
GGAGTCTACTGACGTGTGGAGACGTGGAGGAAAACCCTGGACCTATGGCTCTGCCC
GTGACAGCTTTGCTGCTGCCTTTGGCACTGCTGCTGCATGCTGCTAGACCAGAGGTG
CAGCTGGTTGAATCTGGCGGAGGACTGGTTCAGCCTGGCGGATCTCTGAGACTGTCT
TGTGCCGCCAGCGGCTTCACCTTCAGCAGATACGATATGCACTGGGTCCGACAGGC
CCCTGGCAAAGGACTTGAATGGGTGTCCGTGATCTGGGGCAACGGCAACACACACT
ACCACAGCGCCCTGAAGTCCCGGTTCACCATCTCCAGAGACAACAGCAAGAACACC
CTGTACCTGCAGATGAACAGCCTGAGAGCCGAGGACACCGCCGTGTACTACTGCAC
CCTGAGAATCAAGGATTGGGGCCAGGGCACCATGGTCACCGTTTCTTCTGGAGGCG
GAGGATCTGGTGGCGGAGGAAGTGGCGGAGGCGGTTCTGACGTGGTCATGACACA
GAGCCCTCTGAGCCTGCCTGTGACACTGGGACAGCCTGCCAGCATCAGCTGCAAGT
CTAGCCAGTCTCTGGTGGCCAGCGACGAGAACACCTACCTGAACTGGTTCCAGCAG
AGGCCCGGACAGTCTCCTAGACGGCTGATCTACCAGGTGTCCAAGCTGGATAGCGG
CGTGCCCGATAGATTTTCTGGCAGCGGCTCTGGCACCGACTTCACCCTGAAGATCAG
CAGAGTGGAAGCCGAGGACGTGGGCGTGTACTACTGTCTGCAAGGCATCCATCTGC
CTTGGACCTTTGGCCAGGGCACAAAGCTGGAAATCAAGAATGGCGCTGCACACCCA
TCCGATCCTCTCGAGCTGGTGGTTTCTGGACCTTCTGGCGGCCCTAGCAGCCCTACA
ACAGGACCTACAAGCACAAGCGGCCCTGAGGACCAACCTCTGACACCAACAGGCA
GCGATCCTCAGTCTGGACTGGGGAGACATCTGGGCGTTGTGATCGGCATTCTGGTCG
CCGTGATCCTGCTCCTGTTGCTCCTGCTGCTTCTGTTCCTGATCCTGCGGCACAGAAG
GCAGGGCAAGCACTGGACAAGCACCCAGAGAAAGGCCGACTTTCAGCATCCTGCTG
GCGCCGTTGGACCTGAGCCTACAGATAGAGGACTGCAGTGGCGGTCTAGCCCTGCC
GCTGATGCCCAAGAGGAAAATCTTTACGCCGCCGTGAAGCACACCCAGCCTGAGGA
TGGCGTGGAAATGGACACCAGATCTCCCCACGATGAGGACCCTCAGGCCGTGACAT
ACGCAGAAGTGAAGCACTCCAGACCTCGGAGAGAGATGGCAAGCCCTCCATCTCCT
CTGAGCGGCGAGTTCCTGGACACCAAAGACAGACAGGCCGAAGAGGACAGACAGA
TGGATACCGAAGCCGCCGCTTCTGAAGCCCCACAGGATGTGACATATGCCCAGCTG
CATAGCCTGACACTGCGGAGAGAAGCCACAGAGCCTCCACCTTCTCAAGAAGGCCC
ATCTCCTGCCGTGCCTTCCATCTATGCCACTCTGGCCATTCACGGTTCTGGCCAGTGC
ACAAATTATGCACTGCTGAAGCTCGCCGGGGATGTCGAGAGTAACCCAGGACCTGG
AAGCGGAGAAGGTCGTGGTAGTCTACTAACGTGTGGTGATGTAGAAGAAAATCCTG
GACCTATGCTGCTGCTGGTTACATCTCTGCTGCTGTGCGAGCTGCCCCATCCTGCCTT
TCTGCTTATTCCTGAAGTGCAGCTGGTTCAGTCTGGCGCCGAAGTGAAGAAACCTGG
CAGCAGCGTGAAGGTGTCCTGCAAAGCTTCTGGCGGCACCTTCAGCAGCTACGCCA
TCTCTTGGGTTCGACAGGCCCCTGGACAAGGCCTGGAATGGATGGGAGGCATCATC
CCCATCTTCGGCACCGCCAATTACGCCCAGAAATTCCAGGGCAGAGTGACCATCAC
CGCCGACAAGAGCACAAGCACCGCCTACATGGAACTGAGCAGCCTGAGAAGCGAG
GACACCGCCGTGTACTACTGCGCCACATTTGCCCTGTTCGGCTTCAGAGAGCAGGCC
TTCGATATCTGGGGCCAGGGCACAACCGTGACCGTTTCTAGCGGAGGCGGAGGATC
TGGTGGTGGTGGATCTCAGGTTCAGCTGGTGCAGAGCGGAGCTGAAGTGAAAAAGC
CAGGCTCCTCCGTGAAAGTCTCTTGCAAGGCCAGCGGCTACACCTTTACCGACTACA
ACATGCACTGGGTCCGACAAGCTCCAGGCCAGGGACTTGAGTGGATCGGCTACATC
TACCCCTACAACGGCGGCACCGGCTACAACCAGAAGTTCAAGAGCAAGGCCACAAT
CACAGCCGACGAGTCCACCAACACAGCTTATATGGAACTGTCTAGCCTGCGCTCCG
AGGATACAGCTGTGTACTATTGTGCCAGGGGCAGACCCGCCATGGACTATTGGGGA
CAGGGAACCCTGGTCACAGTGTCATCTGGTGGCGGAGGAAGTGGCGGCGGAGGCTC
TGATATTCAGATGACTCAGAGCCCCAGCAGCCTGTCTGCCTCTGTGGGAGACAGAG
TGACAATTACCTGCCGGGCCAGCGAGAGCGTGGACAATTACGGCATCAGCTTCATG
AACTGGTTCCAGCAGAAGCCCGGCAAGGCCCCTAAGCTGCTGATCTACGCCGCCAG
CAATCAAGGCAGCGGAGTGCCTAGCAGATTTTCCGGCTCTGGCAGCGGCACCGATT
TCACCCTGACCATCAGTAGCCTGCAGCCTGACGACTTCGCCACCTACTACTGCCAGC
AGAGCAAAGAGGTGCCCTGGACCTTTGGACAGGGCACCAAGGTGGAAATCAAAGG
CGGTGGCGGATCTGGCGGAGGTGGCAGTGATATCCAAATGACCCAGTCTCCATCCA
GCCTGAGCGCCAGCGTTGGCGATAGAGTCACAATCACCTGTAGAGCCAGCCAGAGC
ATCAGCTCCTACCTGAATTGGTATCAGCAGAAACCAGGCAAAGCTCCCAAACTCCT
GATCTATGCTGCCTCCAGCCTGCAGAGTGGCGTGCCATCTAGATTCAGCGGCAGCG
GAAGCGGCACAGACTTTACACTGACAATATCTTCCCTGCAGCCAGAGGACCTGGCC
ACATATTATTGCCAGCAGTCCTACAGCACCCCTTTCACCTTCGGACCCGGAACAAAG
GTGGACATTAAGGCCCTGAGCAACAGCATCATGTACTTCAGCCACTTCGTGCCCGTG
TTTCTGCCCGCCAAGCCTACAACAACCCCTGCTCCTAGACCACCTACACCAGCTCCT
ACAATCGCCAGCCAGCCTCTGTCTCTGAGGCCCGAAGCTTGTAGACCAGCTGCTGG
CGGAGCCGTGCATACAAGAGGACTGGATTTTGCCTGCGACATCTATATCTGGGCCC
CTCTGGCTGGCACATGCGGAGTTCTGCTGCTCAGCCTGGTCATCACCCTGTACTGCA
ACCACAGAAGAAGCAAGCGGAGCAGACTGCTGCACAGCGACTACATGAACATGAC
CCCTAGACGGCCCGGACCTACCAGAAAGCACTACCAGCCTTACGCTCCTCCTAGAG
ATTTCGCCGCCTACCGGTCCAGAGTGAAGTTCAGCAGGAGCGCAGACGCCCCCGCG
TACAAGCAGGGCCAGAACCAGCTCTATAACGAGCTCAATCTAGGACGAAGAGAGG
AGTACGATGTTTTGGACAAGAGACGTGGCCGGGACCCTGAGATGGGGGGAAAGCC
GAGAAGGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAAGGGGC
ACGATGGCCTTTACCAGGGTCTCAGTACAGCCACCAAGGACACCTACGACGCCCTT
CACATGCAGGCCCTGCCCCCTCGCtaaTgAggatccggattagtccaatttgttaaagacaggatgggctgcagga
attccgataatcaacctctggattacaaaatttgtgaaagattgactggtattcttaactatgttgctccttttacgctatgtggatacgctgctttaa
tgcctttgtatcatgctattgcttcccgtatggctttcattttctcctccttgtataaatcctggttgctgtctctttatgaggagttgtggcccgttgtc
aggcaacgtggcgtggtgtgcactgtgtttgctgacgcaacccccactggttggggcattgccaccacctgtcagctcctttccgggacttt
cgctttccccctccctattgccacggcggaactcatcgccgcctgccttgcccgctgctggacaggggctcggctgttgggcactgacaatt
ccgtggtgttgtcggggaagctgacgtcctttccatggctgctcgcctgtgttgccacctggattctgcgcgggacgtccttctgctacgtcc
cttcggccctcaatccagcggaccttccttcccgcggcctgctgccggctctgcggcctcttccgcgtcttcgccttcgccctcagacgagt
cggatctccctttgggccgcctccccgcctggagaattcgatatcagtggtccaggctctagttttgactcaacaatatcaccagctgaagcc
tatagagtacgagccatagataaaataaaagattttatttagtctccagaaaaaggggggaatgaaagaccccacctgtaggtttggcaagc
tagcaataaaagagcccacaacccctcactcggggcgccagtcctccgattgactgagtcgcccggccgcttcgagcagacatgataag
atacattgatgagtttggacaaaccacaactagaatgcagtgaaaaaaatgctttatttgtgaaatttgtgatgctattgctttatttgtaaccatta
taagctgcaataaacaagttaacaacaacaattgcattcattttatgtttcaggttcagggggagatgtgggaggttttttaaagcaagtaaaac
ctctacaaatgtggtaaaatcgataaggatcgggtacccgtgtatccaataaaccctcttgcagttgcatccgacttgtggtctcgctgttcctt
gggagggtctcctctgagtgattgactacccgtcagcgggggtctttcacacatgcagcatgtatcaaaattaatttggttttttttcttaagctgt
gccttctagttgccagccatctgttgtttgcccctcccccgtgccttccttgaccctggaaggtgccactcccactgtcctttcctaataaaatga
ggaaattgcatcgcattgtctgagtaggtgtcattctattctggggggtggggggggcaggacagcaagggggaggattgggaagacaa
tagcaggcatgctggggatgcggtgggctctatggagatcccgcggtacctcgcgaatgcatctagatccaatggcctttttggcccagac
atgataagatacattgatgagtttggacaaaccacaactagaatgcagtgaaaaaaatgctttatttgtgaaatttgtgatgctattgctttatttgt
aaccattataagctgcaataaacaagttgcggccgcttagccctcccacacataaccagagggcagcaattcacgaatcccaactgccgtc
ggctgtccatcactgtccttcactatggctttgatcccaggatgcagatcgagaagcacctgtcggcaccgtccgcaggggctcaagatgc
ccctgttctcatttccgatcgcgacgatacaagtcaggttgccagctgccgcagcagcagcagtgcccagcaccacgagttctgcacaag
gtcccccagtaaaatgatatacattgacaccagtgaagatgcggccgtcgctagagagagctgcgctggcgacgctgtagtcttcagagat
ggggatgctgttgattgtagccgttgctctttcaatgagggtggattcttcttgagacaaaggcttggccatgcggccgccgctcggtgttcg
aggccacacgcgtcaccttaatatgcgaagtggacctcggaccgcgccgccccgactgcatctgcgtgttcgaattcgccaatgacaaga
cgctgggcggggtttgtgtcatcatagaactaaagacatgcaaatatatttcttccggggggtaccggcctttttggccATTGGatcgga
tctggccaaaaaggcccttaagtatttacattaaatggccatagtacttaaagttacattggcttccttgaaataaacatggagtattcagaatgtgtca
taaatatttctaattttaagatagtatctccattggctttctactttttcttttatttttttttgtcctctgtcttccatttgttgttgttgttgtttgtt
tgtttgtttgttggttggttggttaatttttttttaaagatcctacactatagttcaagctagactattagctactctgtaacccagggtgaccttgaagtca
tgggtagcctgctgttttagccttcccacatctaagattacaggtatgagctatcatttttggtatattgattgattgattgattgatgtgtgtgtgtg
tgattgtgtttgtgtgtgtgaTtgtgTaTatgtgtgtatggTtgtgtgtgaTtgtgtgtatgtatgTTtgtgtgtgaTtgTgtgtgtgtgaTt
gtgcatgtgtgtgtgtgtgaTtgtgtTtatgtgtatgaTtgtgtgtgtgtgtgtgtgtgtgtgtgtgtgtgtgtgtgtgtgtgtgttgtgTaTaT
atatttatggtagtgagagGcaacgctccggctcaggtgtcaggttggtttttgagacagagtctttcacttagcttggaattcactggccgtc
gttttacaacgtcgtgactgggaaaaccctggcgttacccaacttaatcgccttgcagcacatccccctttcgccagctggcgtaatagcgaa
gaggcccgcaccgatcgcccttcccaacagttgcgcagcctgaatggcgaatggcgcctgatgcggtattttctccttacgcatctgtgcgg
tatttcacaccgcatatggtgcactctcagtacaatctgctctgatgccgcatagttaagccagccccgacacccgccaacacccgctgacg
cgccctgacgggcttgtctgctcccggcatccgcttacagacaagctgtgaccgtctccgggagctgcatgtgtcagaggttttcaccgtca
tcaccgaaacgcgcgagacgaaagggcctcgtgatacgcctatttttataggttaatgtcatgataataatggtttcttagacgtcaggtggca
cttttcggggaaatgtgcgcggaacccctatttgtttatttttctaaatacattcaaatatgtatccgctcatgagacaataaccctgataaatgctt
caataatattgaaaaaggaagagtatgagtattcaacatttccgtgtcgcccttattcccttttttgcggcattttgccttcctgtttttgctcaccca
gaaacgctggtgaaagtaaaagatgctgaagatcagttgggtgcacgagtgggttacatcgaactggatctcaacagcggtaagatccttg
agagttttcgccccgaagaacgttttccaatgatgagcacttttaaagttctgctatgtggcgcggtattatcccgtattgacgccgggcaaga
gcaactcggtcgccgcatacactattctcagaatgacttggttgagtactcaccagtcacagaaaagcatcttacggatggcatgacagtaa
gagaattatgcagtgctgccataaccatgagtgataacactgcggccaacttacttctgacaacgatcggaggaccgaaggagctaaccg
cttttttgcacaacatgggggatcatgtaactcgccttgatcgttgggaaccggagctgaatgaagccataccaaacgacgagcgtgacac
cacgatgcctgtagcaatggcaacaacgttgcgcaaactattaactggcgaactacttactctagcttcccggcaacaattaatagactggat
ggaggcggataaagttgcaggaccacttctgcgctcggcccttccggctggctggtttattgctgataaatctggagccggtgagcgtggg
tctcgcggtatcattgcagcactggggccagatggtaagccccccgtatcgtagttatctacacgacggggagtcaggcaactatggatg
aacgaaatagacagatcgctgagataggtgcctcactgattaagcattggtaactgtcagaccaagtttactcatatatactttagattgatttaa
aacttcatttttaatttaaaaggatctaggtgaagatcctttttgataatctcatgaccaaaatcccttaacgtgagttttcgttccactgagcgtca
gaccccgtagaaaagatcaaaggatcttcttgagatcctttttttctgcgcgtaatctgctgcttgcaaacaaaaaaaccaccgctaccagcg
gtggtttgtttgccggatcaagagctaccaactctttttccgaaggtaactggcttcagcagagcgcagataccaaatactgtccttctagtgta
gccgtagttaggccaccacttcaagaactctgtagcaccgcctacatacctcgctctgctaatcctgttaccagtggctgctgccagtggcga
taagtcgtgtcttaccgggttggactcaagacgatagttaccggataaggcgcagcggtcgggctgaacggggggttcgtgcacacagcc
cagcttggagcgaacgacctacaccgaactgagatacctacagcgtgagctatgagaaagcgccacgcttcccgaagggagaaaggcg
gacaggtatccggtaagcggcagggtcggaacaggagagcgcacgagggagcttccagggggaaacgcctggtatctttatagtcctgt
cgggtttcgccacctctgacttgagcgtcgatttttgtgatgctcgtcaggggggcggagcctatggaaaaacgccagcaacgcggcctttt
tacggttcctggccttttgctggccttttgctcacatgttctttcctgcgttatcccctgattctgtggataaccgtattaccgcctttgagtgagct
gataccgctcgccgcagccgaacgaccgagcgcagcgagtcagtgagcgaggaagcggaagagcgcccaatacgcaaaccgcctct
ccccgcgcgttggccgattcattaatgcagctggcacgacaggtttcccgactggaaagcgggcagtgagcgcaacgcaattaatgtgag
ttagctcactcattaggcaccccaggctttacactttatgcttccggctcgtatgttgtgtggaattgtgagcggataacaatttcacacaggaa
acagctatgaccatgattacgcc
SB08288; SINvec SV40 (SEQ ID NO: 432)
aagcttgaattcgagcttgcatgcctgcaggtcgttacataacttacggtaaatggcccgcctggctgaccgcccaacgacccccgcccatt
gacgtcaataatgacgtatgttcccatagtaacgccaatagggactttccattgacgtcaatgggtggagtatttacggtaaactgcccacttg
gcagtacatcaagtgtatcatatgccaagtacgccccctattgacgtcaatgacggtaaatggcccgcctggcattatgcccagtacatgac
cttatgggactttcctacttggcagtacatctacgtattagtcatcgctattaccatggtgatgcggttttggcagtacatcaatgggcgtggata
gcggtttgactcacggggatttccaagtctccaccccattgacgtcaatgggagtttgttttggcaccaaaatcaacgggactttccaaaatgt
cgtaacaactccgccccattgacgcaaatgggcggtaggcgtgtacggtgggaggtctatataagcagagctcaataaaagagcccaca
acccctcactcggcgcgccagtcctccgattgactgagtcgcccgggtacccgtgtatccaataaaccctcttgcagttgcatccgacttgtg
gtctcgctgttccttgggagggtctcctctgagtgattgactacccgtcagcgggggtctttcatttgggggctcgtccgagatcgggagacc
cctgcccagggaccaccgacccaccaccgggaggtaagctggccagcaacttatctgtgtctgtccgattgtctagtgtctatgactgatttt
atgcgcctgcgtcggtactagttagctaactagctctgtatctggcggacccgtggtggaactgacgagttcggaacacccggccgcaacc
ctgggagacgtcccagggacttcgggggccgtttttgtggcccgacctgagtcctaaaatcccgatcgtttaggactctttggtgcaccccc
cttagaggagggatatgtggttctggtaggagacgagaacctaaaacagttcccgcctccgtctgaatttttgctttcggtttgggaccgaag
ccgcgccgcgcgtcttgtctgctgcagcatcgttctgtgttgtctctgtctgactgtgtttctgtatttgtctgaaaatatgggccccccctcgag
CTGTGGAATGTGTGTCAGTTAGGGTGTGGAAAGTCCCCAGGCTCCCCAGCAGGCAG
AAGTATGCAAAGCATGCATCTCAATTAGTCAGCAACCAGGTGTGGAAAGTCCCCAG
GCTCCCCAGCAGGCAGAAGTATGCAAAGCATGCATCTCAATTAGTCAGCAACCATA
GTCCCGCCCCTAACTCCGCCCATCCCGCCCCTAACTCCGCCCAGTTCCGCCCATTCT
CCGCCCCATGGCTGACTAATTTTTTTTATTTATGCAGAGGCCGAGGCCGCCTCTGCC
TCTGAGCTATTCCAGAAGTAGTGAGGAGGCTTTTTTGGAGGCCTAGGCTTTTGCAAA
AAGCTGGATCCGCGTCAAGTGGAGCAAGGCAGGTGGACAGTCTCGAggccgccaccATG
GACTGGACTTGGATACTCTTTCTGGTCGCTGCCGCCACACGGGTGCACTCTAATTGG
GTCAACGTGATCAGCGACCTGAAGAAGATCGAGGACCTGATCCAGAGCATGCACAT
CGACGCCACACTGTACACCGAGTCCGATGTGCACCCTAGCTGCAAAGTGACCGCCA
TGAAGTGCTTTCTGCTGGAACTGCAAGTGATCAGCCTGGAAAGCGGCGACGCCAGC
ATCCACGATACCGTGGAAAATCTGATCATCCTGGCCAACAACAGCCTGTCCAGCAA
CGGCAATGTGACCGAGAGCGGCTGCAAAGAGTGCGAGGAACTGGAAGAGAAGAAC
ATCAAAGAGTTTCTGCAGAGCTTCGTCCACATCGTGCAGATGTTCATCAACACCTCA
TCAGGCGGCGGTGGTAGTGGAGGCGGAGGCTCAGGCGTGACCCCTGAGCCTATCTT
CAGCCTGATCGGCGGAGGTTCCGGAGGTGGCGGTTCCGGCGGAGGATCTCTTCAAT
TGCTGCCTAGCTGGGCCATCACACTGATCTCCGTGAACGGCATCTTCGTGATCTGCT
GCCTGACCTACTGCTTCGCCCCTAGATGCAGAGAGCGGAGAAGAAACGAGCGGCTG
AGAAGAGAAAGCGTGCGGCCTGTGGGTAGCGGCCAGTGTACCAACTACGCCCTGCT
GAAACTGGCCGGCGACGTGGAATCTAATCCTGGACCTGGATCTGGCGAGGGACGCG
GGAGTCTACTGACGTGTGGAGACGTGGAGGAAAACCCTGGACCTATGGCTCTGCCC
GTGACAGCTTTGCTGCTGCCTTTGGCACTGCTGCTGCATGCTGCTAGACCAGAGGTG
CAGCTGGTTGAATCTGGCGGAGGACTGGTTCAGCCTGGCGGATCTCTGAGACTGTCT
TGTGCCGCCAGCGGCTTCACCTTCAGCAGATACGATATGCACTGGGTCCGACAGGC
CCCTGGCAAAGGACTTGAATGGGTGTCCGTGATCTGGGGCAACGGCAACACACACT
ACCACAGCGCCCTGAAGTCCCGGTTCACCATCTCCAGAGACAACAGCAAGAACACC
CTGTACCTGCAGATGAACAGCCTGAGAGCCGAGGACACCGCCGTGTACTACTGCAC
CCTGAGAATCAAGGATTGGGGCCAGGGCACCATGGTCACCGTTTCTTCTGGAGGCG
GAGGATCTGGTGGCGGAGGAAGTGGCGGAGGCGGTTCTGACGTGGTCATGACACA
GAGCCCTCTGAGCCTGCCTGTGACACTGGGACAGCCTGCCAGCATCAGCTGCAAGT
CTAGCCAGTCTCTGGTGGCCAGCGACGAGAACACCTACCTGAACTGGTTCCAGCAG
AGGCCCGGACAGTCTCCTAGACGGCTGATCTACCAGGTGTCCAAGCTGGATAGCGG
CGTGCCCGATAGATTTTCTGGCAGCGGCTCTGGCACCGACTTCACCCTGAAGATCAG
CAGAGTGGAAGCCGAGGACGTGGGCGTGTACTACTGTCTGCAAGGCATCCATCTGC
CTTGGACCTTTGGCCAGGGCACAAAGCTGGAAATCAAGAATGGCGCTGCACACCCA
TCCGATCCTCTCGAGCTGGTGGTTTCTGGACCTTCTGGCGGCCCTAGCAGCCCTACA
ACAGGACCTACAAGCACAAGCGGCCCTGAGGACCAACCTCTGACACCAACAGGCA
GCGATCCTCAGTCTGGACTGGGGAGACATCTGGGCGTTGTGATCGGCATTCTGGTCG
CCGTGATCCTGCTCCTGTTGCTCCTGCTGCTTCTGTTCCTGATCCTGCGGCACAGAAG
GCAGGGCAAGCACTGGACAAGCACCCAGAGAAAGGCCGACTTTCAGCATCCTGCTG
GCGCCGTTGGACCTGAGCCTACAGATAGAGGACTGCAGTGGCGGTCTAGCCCTGCC
GCTGATGCCCAAGAGGAAAATCTTTACGCCGCCGTGAAGCACACCCAGCCTGAGGA
TGGCGTGGAAATGGACACCAGATCTCCCCACGATGAGGACCCTCAGGCCGTGACAT
ACGCAGAAGTGAAGCACTCCAGACCTCGGAGAGAGATGGCAAGCCCTCCATCTCCT
CTGAGCGGCGAGTTCCTGGACACCAAAGACAGACAGGCCGAAGAGGACAGACAGA
TGGATACCGAAGCCGCCGCTTCTGAAGCCCCACAGGATGTGACATATGCCCAGCTG
CATAGCCTGACACTGCGGAGAGAAGCCACAGAGCCTCCACCTTCTCAAGAAGGCCC
ATCTCCTGCCGTGCCTTCCATCTATGCCACTCTGGCCATTCACGGTTCTGGCCAGTGC
ACAAATTATGCACTGCTGAAGCTCGCCGGGGATGTCGAGAGTAACCCAGGACCTGG
AAGCGGAGAAGGTCGTGGTAGTCTACTAACGTGTGGTGATGTAGAAGAAAATCCTG
GACCTATGCTGCTGCTGGTTACATCTCTGCTGCTGTGCGAGCTGCCCCATCCTGCCTT
TCTGCTTATTCCTGAAGTGCAGCTGGTTCAGTCTGGCGCCGAAGTGAAGAAACCTGG
CAGCAGCGTGAAGGTGTCCTGCAAAGCTTCTGGCGGCACCTTCAGCAGCTACGCCA
TCTCTTGGGTTCGACAGGCCCCTGGACAAGGCCTGGAATGGATGGGAGGCATCATC
CCCATCTTCGGCACCGCCAATTACGCCCAGAAATTCCAGGGCAGAGTGACCATCAC
CGCCGACAAGAGCACAAGCACCGCCTACATGGAACTGAGCAGCCTGAGAAGCGAG
GACACCGCCGTGTACTACTGCGCCACATTTGCCCTGTTCGGCTTCAGAGAGCAGGCC
TTCGATATCTGGGGCCAGGGCACAACCGTGACCGTTTCTAGCGGAGGCGGAGGATC
TGGTGGTGGTGGATCTCAGGTTCAGCTGGTGCAGAGCGGAGCTGAAGTGAAAAAGC
CAGGCTCCTCCGTGAAAGTCTCTTGCAAGGCCAGCGGCTACACCTTTACCGACTACA
ACATGCACTGGGTCCGACAAGCTCCAGGCCAGGGACTTGAGTGGATCGGCTACATC
TACCCCTACAACGGCGGCACCGGCTACAACCAGAAGTTCAAGAGCAAGGCCACAAT
CACAGCCGACGAGTCCACCAACACAGCTTATATGGAACTGTCTAGCCTGCGCTCCG
AGGATACAGCTGTGTACTATTGTGCCAGGGGCAGACCCGCCATGGACTATTGGGGA
CAGGGAACCCTGGTCACAGTGTCATCTGGTGGCGGAGGAAGTGGCGGCGGAGGCTC
TGATATTCAGATGACTCAGAGCCCCAGCAGCCTGTCTGCCTCTGTGGGAGACAGAG
TGACAATTACCTGCCGGGCCAGCGAGAGCGTGGACAATTACGGCATCAGCTTCATG
AACTGGTTCCAGCAGAAGCCCGGCAAGGCCCCTAAGCTGCTGATCTACGCCGCCAG
CAATCAAGGCAGCGGAGTGCCTAGCAGATTTTCCGGCTCTGGCAGCGGCACCGATT
TCACCCTGACCATCAGTAGCCTGCAGCCTGACGACTTCGCCACCTACTACTGCCAGC
AGAGCAAAGAGGTGCCCTGGACCTTTGGACAGGGCACCAAGGTGGAAATCAAAGG
CGGTGGCGGATCTGGCGGAGGTGGCAGTGATATCCAAATGACCCAGTCTCCATCCA
GCCTGAGCGCCAGCGTTGGCGATAGAGTCACAATCACCTGTAGAGCCAGCCAGAGC
ATCAGCTCCTACCTGAATTGGTATCAGCAGAAACCAGGCAAAGCTCCCAAACTCCT
GATCTATGCTGCCTCCAGCCTGCAGAGTGGCGTGCCATCTAGATTCAGCGGCAGCG
GAAGCGGCACAGACTTTACACTGACAATATCTTCCCTGCAGCCAGAGGACCTGGCC
ACATATTATTGCCAGCAGTCCTACAGCACCCCTTTCACCTTCGGACCCGGAACAAAG
GTGGACATTAAGGCCCTGAGCAACAGCATCATGTACTTCAGCCACTTCGTGCCCGTG
TTTCTGCCCGCCAAGCCTACAACAACCCCTGCTCCTAGACCACCTACACCAGCTCCT
ACAATCGCCAGCCAGCCTCTGTCTCTGAGGCCCGAAGCTTGTAGACCAGCTGCTGG
CGGAGCCGTGCATACAAGAGGACTGGATTTTGCCTGCGACATCTATATCTGGGCCC
CTCTGGCTGGCACATGCGGAGTTCTGCTGCTCAGCCTGGTCATCACCCTGTACTGCA
ACCACAGAAGAAGCAAGCGGAGCAGACTGCTGCACAGCGACTACATGAACATGAC
CCCTAGACGGCCCGGACCTACCAGAAAGCACTACCAGCCTTACGCTCCTCCTAGAG
ATTTCGCCGCCTACCGGTCCAGAGTGAAGTTCAGCAGGAGCGCAGACGCCCCCGCG
TACAAGCAGGGCCAGAACCAGCTCTATAACGAGCTCAATCTAGGACGAAGAGAGG
AGTACGATGTTTTGGACAAGAGACGTGGCCGGGACCCTGAGATGGGGGGAAAGCC
GAGAAGGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAAGGGGC
ACGATGGCCTTTACCAGGGTCTCAGTACAGCCACCAAGGACACCTACGACGCCCTT
CACATGCAGGCCCTGCCCCCTCGCtaaTgAggatccggattagtccaatttgttaaagacaggatgggctgcagga
attccgataatcaacctctggattacaaaatttgtgaaagattgactggtattcttaactatgttgctccttttacgctatgtggatacgctgctttaa
tgcctttgtatcatgctattgcttcccgtatggctttcattttctcctccttgtataaatcctggttgctgtctctttatgaggagttgtggcccgttgtc
aggcaacgtggcgtggtgtgcactgtgtttgctgacgcaacccccactggttggggcattgccaccacctgtcagctcctttccgggacttt
cgctttccccctccctattgccacggcggaactcatcgccgcctgccttgcccgctgctggacaggggctcggctgttgggcactgacaatt
ccgtggtgttgtcggggaagctgacgtcctttccatggctgctcgcctgtgttgccacctggattctgcgcgggacgtccttctgctacgtcc
cttcggccctcaatccagcggaccttccttcccgcggcctgctgccggctctgcggcctcttccgcgtcttcgccttcgccctcagacgagt
cggatctccctttgggccgcctccccgcctggagaattcgatatcagtggtccaggctctagttttgactcaacaatatcaccagctgaagcc
tatagagtacgagccatagataaaataaaagattttatttagtctccagaaaaaggggggaatgaaagaccccacctgtaggtttggcaagc
tagcaataaaagagcccacaacccctcactcggggcgccagtcctccgattgactgagtcgcccggccgcttcgagcagacatgataag
atacattgatgagtttggacaaaccacaactagaatgcagtgaaaaaaatgctttatttgtgaaatttgtgatgctattgctttatttgtaaccatta
taagctgcaataaacaagttaacaacaacaattgcattcattttatgtttcaggttcagggggagatgtgggaggttttttaaagcaagtaaaac
ctctacaaatgtggtaaaatcgataaggatcgggtacccgtgtatccaataaaccctcttgcagttgcatccgacttgtggtctcgctgttcctt
gggagggtctcctctgagtgattgactacccgtcagcgggggtctttcacacatgcagcatgtatcaaaattaatttggttttttttcttaagctgt
gccttctagttgccagccatctgttgtttgcccctcccccgtgccttccttgaccctggaaggtgccactcccactgtcctttcctaataaaatga
ggaaattgcatcgcattgtctgagtaggtgtcattctattctggggggtggggggggcaggacagcaagggggaggattgggaagacaa
tagcaggcatgctggggatgcggtgggctctatggagatcccgcggtacctcgcgaatgcatctagatccaatggcctttttggcccagac
atgataagatacattgatgagtttggacaaaccacaactagaatgcagtgaaaaaaatgctttatttgtgaaatttgtgatgctattgctttatttgt
aaccattataagctgcaataaacaagttgcggccgcttagccctcccacacataaccagagggcagcaattcacgaatcccaactgccgtc
ggctgtccatcactgtccttcactatggctttgatcccaggatgcagatcgagaagcacctgtcggcaccgtccgcaggggctcaagatgc
ccctgttctcatttccgatcgcgacgatacaagtcaggttgccagctgccgcagcagcagcagtgcccagcaccacgagttctgcacaag
gtcccccagtaaaatgatatacattgacaccagtgaagatgcggccgtcgctagagagagctgcgctggcgacgctgtagtcttcagagat
ggggatgctgttgattgtagccgttgctctttcaatgagggtggattcttcttgagacaaaggcttggccatgcggccgccgctcggtgttcg
aggccacacgcgtcaccttaatatgcgaagtggacctcggaccgcgccgccccgactgcatctgcgtgttcgaattcgccaatgacaaga
cgctgggcggggtttgtgtcatcatagaactaaagacatgcaaatatatttcttccggggggtaccggcctttttggccATTGGatcgga
tctggccaaaaaggcccttaagtatttacattaaatggccatagtacttaaagttacattggcttccttgaaataaacatggagtattcagaatgtgtca
taaatatttctaattttaagatagtatctccattggctttctactttttcttttatttttttttgtcctctgtcttccatttgttgttgttgttgtttgtt
tgtttgtttgttggttggttggttaatttttttttaaagatcctacactatagttcaagctagactattagctactctgtaacccagggtgaccttgaagtca
tgggtagcctgctgttttagccttcccacatctaagattacaggtatgagctatcatttttggtatattgattgattgattgattgatgtgtgtgtgtg
tgattgtgtttgtgtgtgtgaTtgtgTaTatgtgtgtatggTtgtgtgtgaTtgtgtgtatgtatgTTtgtgtgtgaTtgTgtgtgtgtgaTt
gtgcatgtgtgtgtgtgtgaTtgtgtTtatgtgtatgaTtgtgtgtgtgtgtgtgtgtgtgtgtgtgtgtgtgtgtgtgtgtgtgttgtgTaTaT
atatttatggtagtgagagGcaacgctccggctcaggtgtcaggttggtttttgagacagagtctttcacttagcttggaattcactggccgtc
gttttacaacgtcgtgactgggaaaaccctggcgttacccaacttaatcgccttgcagcacatccccctttcgccagctggcgtaatagcgaa
gaggcccgcaccgatcgcccttcccaacagttgcgcagcctgaatggcgaatggcgcctgatgcggtattttctccttacgcatctgtgcgg
tatttcacaccgcatatggtgcactctcagtacaatctgctctgatgccgcatagttaagccagccccgacacccgccaacacccgctgacg
cgccctgacgggcttgtctgctcccggcatccgcttacagacaagctgtgaccgtctccgggagctgcatgtgtcagaggttttcaccgtca
tcaccgaaacgcgcgagacgaaagggcctcgtgatacgcctatttttataggttaatgtcatgataataatggtttcttagacgtcaggtggca
cttttcggggaaatgtgcgcggaacccctatttgtttatttttctaaatacattcaaatatgtatccgctcatgagacaataaccctgataaatgctt
caataatattgaaaaaggaagagtatgagccatattcaacgggaaacgtcgaggccgcgattaaattccaacatggatgctgatttatatgg
gtataaatgggctcgcgataatgtcgggcaatcaggtgcgacaatctatcgcttgtatgggaagcccgatgcgccagagttgtttctgaaac
atggcaaaggtagcgttgccaatgatgttacagatgagatggtcagactaaactggctgacggaatttatgcctcttccgaccatcaagcatt
ttatccgtactcctgatgatgcatggttactcaccactgcgatccccggaaaaacagcattccaggtattagaagaatatcctgattcaggtga
aaatattgttgatgcgctggcagtgttcctgcgccggttgcattcgattcctgtttgtaattgtccttttaacagcgatcgcgtatttcgtctcgctc
aggcgcaatcacgaatgaataacggtttggttgatgcgagtgattttgatgacgagcgtaatggctggcctgttgaacaagtctggaaagaa
atgcataaacttttgccattctcaccggattcagtcgtcactcatggtgatttctcacttgataaccttatttttgacgaggggaaattaataggttg
tattgatgttggacgagtcggaatcgcagaccgataccaggatcttgccatcctatggaactgcctcggtgagttttctccttcattacagaaa
cggctttttcaaaaatatggtattgataatcctgatatgaataaattgcagtttcatttgatgctcgatgagtttttctaactgtcagaccaagtttac
tcatatatactttagattgatttaaaacttcatttttaatttaaaaggatctaggtgaagatcctttttgataatctcatgaccaaaatcccttaacgtg
agttttcgttccactgagcgtcagaccccgtagaaaagatcaaaggatcttcttgagatcctttttttctgcgcgtaatctgctgcttgcaaacaa
aaaaaccaccgctaccagcggtggtttgtttgccggatcaagagctaccaactctttttccgaaggtaactggcttcagcagagcgcagata
ccaaatactgtccttctagtgtagccgtagttaggccaccacttcaagaactctgtagcaccgcctacatacctcgctctgctaatcctgttacc
agtggctgctgccagtggcgataagtcgtgtcttaccgggttggactcaagacgatagttaccggataaggcgcagcggtcgggctgaac
ggggggttcgtgcacacagcccagcttggagcgaacgacctacaccgaactgagatacctacagcgtgagctatgagaaagcgccacg
cttcccgaagggagaaaggcggacaggtatccggtaagcggcagggtcggaacaggagagcgcacgagggagcttccagggggaa
acgcctggtatctttatagtcctgtcgggtttcgccacctctgacttgagcgtcgatttttgtgatgctcgtcaggggggcggagcctatggaa
aaacgccagcaacgcggcctttttacggttcctggccttttgctggccttttgctcacatgttctttcctgcgttatcccctgattctgtggataac
cgtattaccgcctttgagtgagctgataccgctcgccgcagccgaacgaccgagcgcagcgagtcagtgagcgaggaagcggaagagc
gcccaatacgcaaaccgcctctccccgcgcgttggccgattcattaatgcagctggcacgacaggtttcccgactggaaagcgggcagtg
agcgcaacgcaattaatgtgagttagctcactcattaggcaccccaggctttacactttatgcttccggctcgtatgttgtgt ggaattgtgagc
ggataacaatttcacacaggaaacagctatgaccatgattacgcc
SB07412_1; SINvec SV40 (SEQ ID NO: 433)
ACTACAACATGCACTGGGTCCGACAAGCTCCAGGCCAGGGACTTGAGTGGATCGGC
TACATCTACCCCTACAACGGCGGCACCGGCTACAACCAGAAGTTCAAGAGCAAGGC
CACAATCACAGCCGACGAGTCCACCAACACAGCTTATATGGAACTGTCTAGCCTGC
GCTCCGAGGATACAGCTGTGTACTATTGTGCCAGGGGCAGACCCGCCATGGACTAT
TGGGGACAGGGAACCCTGGTCACAGTGTCATCTGGTGGCGGAGGAAGTGGCGGCG
GAGGCTCTGATATTCAGATGACTCAGAGCCCCAGCAGCCTGTCTGCCTCTGTGGGA
GACAGAGTGACAATTACCTGCCGGGCCAGCGAGAGCGTGGACAATTACGGCATCAG
CTTCATGAACTGGTTCCAGCAGAAGCCCGGCAAGGCCCCTAAGCTGCTGATCTACG
CCGCCAGCAATCAAGGCAGCGGAGTGCCTAGCAGATTTTCCGGCTCTGGCAGCGGC
ACCGATTTCACCCTGACCATCAGTAGCCTGCAGCCTGACGACTTCGCCACCTACTAC
TGCCAGCAGAGCAAAGAGGTGCCCTGGACCTTTGGACAGGGCACCAAGGTGGAAA
TCAAAGGCGGTGGCGGATCTGGCGGAGGTGGCAGTGATATCCAAATGACCCAGTCT
CCATCCAGCCTGAGCGCCAGCGTTGGCGATAGAGTCACAATCACCTGTAGAGCCAG
CCAGAGCATCAGCTCCTACCTGAATTGGTATCAGCAGAAACCAGGCAAAGCTCCCA
AACTCCTGATCTATGCTGCCTCCAGCCTGCAGAGTGGCGTGCCATCTAGATTCAGCG
GCAGCGGAAGCGGCACAGACTTTACACTGACAATATCTTCCCTGCAGCCAGAGGAC
CTGGCCACATATTATTGCCAGCAGTCCTACAGCACCCCTTTCACCTTCGGACCCGGA
ACAAAGGTGGACATTAAGGCCCTGAGCAACAGCATCATGTACTTCAGCCACTTCGT
GCCCGTGTTTCTGCCCGCCAAGCCTACAACAACCCCTGCTCCTAGACCACCTACACC
AGCTCCTACAATCGCCAGCCAGCCTCTGTCTCTGAGGCCCGAAGCTTGTAGACCAGC
TGCTGGCGGAGCCGTGCATACAAGAGGACTGGATTTTGCCTGCGACATCTATATCTG
GGCCCCTCTGGCTGGCACATGCGGAGTTCTGCTGCTCAGCCTGGTCATCACCCTGTA
CTGCAACCACAGAAGAAGCAAGCGGAGCAGACTGCTGCACAGCGACTACATGAAC
ATGACCCCTAGACGGCCCGGACCTACCAGAAAGCACTACCAGCCTTACGCTCCTCC
TAGAGATTTCGCCGCCTACCGGTCCAGAGTGAAGTTCAGCAGGAGCGCAGACGCCC
CCGCGTACAAGCAGGGCCAGAACCAGCTCTATAACGAGCTCAATCTAGGACGAAGA
GAGGAGTACGATGTTTTGGACAAGAGACGTGGCCGGGACCCTGAGATGGGGGGAA
AGCCGAGAAGGAAGAACCCTCAGGAAGGCCTGTACAATGAACTGCAGAAAGATAA
GATGGCGGAGGCCTACAGTGAGATTGGGATGAAAGGCGAGCGCCGGAGGGGCAAG
GGGCACGATGGCCTTTACCAGGGTCTCAGTACAGCCACCAAGGACACCTACGACGC
CCTTCACATGCAGGCCCTGCCCCCTCGCTAATGAGGATCCGGATTAGTCCAATTTGT
TAAAGACAGGATGGGCTGCAGGAATTCCGATAATCAACCTCTGGATTACAAAATTT
GTGAAAGATTGACTGGTATTCTTAACTATGTTGCTCCTTTTACGCTATGTGGATACG
CTGCTTTAATGCCTTTGTATCATGCTATTGCTTCCCGTATGGCTTTCATTTTCTCCTCC
TTGTATAAATCCTGGTTGCTGTCTCTTTATGAGGAGTTGTGGCCCGTTGTCAGGCAA
CGTGGCGTGGTGTGCACTGTGTTTGCTGACGCAACCCCCACTGGTTGGGGCATTGCC
ACCACCTGTCAGCTCCTTTCCGGGACTTTCGCTTTCCCCCTCCCTATTGCCACGGCGG
AACTCATCGCCGCCTGCCTTGCCCGCTGCTGGACAGGGGCTCGGCTGTTGGGCACTG
ACAATTCCGTGGTGTTGTCGGGGAAGCTGACGTCCTTTCCATGGCTGCTCGCCTGTG
TTGCCACCTGGATTCTGCGCGGGACGTCCTTCTGCTACGTCCCTTCGGCCCTCAATC
CAGCGGACCTTCCTTCCCGCGGCCTGCTGCCGGCTCTGCGGCCTCTTCCGCGTCTTC
GCCTTCGCCCTCAGACGAGTCGGATCTCCCTTTGGGCCGCCTCCCCGCCTGGAGAAT
TCGATATCAGTGGTCCAGGCTCTAGTTTTGACTCAACAATATCACCAGCTGAAGCCT
ATAGAGTACGAGCCATAGATAAAATAAAAGATTTTATTTAGTCTCCAGAAAAAGGG
GGGAATGAAAGACCCCACCTGTAGGTTTGGCAAGCTAGCAATAAAAGAGCCCACA
ACCCCTCACTCGGGGCGCCAGTCCTCCGATTGACTGAGTCGCCCGGCCGCTTCGAGC
AGACATGATAAGATACATTGATGAGTTTGGACAAACCACAACTAGAATGCAGTGAA
AAAAATGCTTTATTTGTGAAATTTGTGATGCTATTGCTTTATTTGTAACCATTATAAG
CTGCAATAAACAAGTTAACAACAACAATTGCATTCATTTTATGTTTCAGGTTCAGGG
GGAGATGTGGGAGGTTTTTTAAAGCAAGTAAAACCTCTACAAATGTGGTAAAATCG
ATAAGGATCGGGTACCCGTGTATCCAATAAACCCTCTTGCAGTTGCATCCGACTTGT
GGTCTCGCTGTTCCTTGGGAGGGTCTCCTCTGAGTGATTGACTACCCGTCAGCGGGG
GTCTTTCACACATGCAGCATGTATCAAAATTAATTTGGTTTTTTTTCTTAAGCTGTGC
CTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCT
GAGTAGGTGTCATTCTATTCTGGGGGGTGGGGTGGGGCAGGACAGCAAGGGGGAG
GATTGGGAAGACAATAGCAGGCATGCTGGGGATGCGGTGGGCTCTATGGAGATCCC
GCGGTACCTCGCGAATGCATCTAGATCCAATGGCCTTTTTGGCCCAGACATGATAAG
ATACATTGATGAGTTTGGACAAACCACAACTAGAATGCAGTGAAAAAAATGCTTTA
TTTGTGAAATTTGTGATGCTATTGCTTTATTTGTAACCATTATAAGCTGCAATAAAC
AAGTTGCGGCCGCTTAGCCCTCCCACACATAACCAGAGGGCAGCAATTCACGAATC
CCAACTGCCGTCGGCTGTCCATCACTGTCCTTCACTATGGCTTTGATCCCAGGATGC
AGATCGAGAAGCACCTGTCGGCACCGTCCGCAGGGGCTCAAGATGCCCCTGTTCTC
ATTTCCGATCGCGACGATACAAGTCAGGTTGCCAGCTGCCGCAGCAGCAGCAGTGC
CCAGCACCACGAGTTCTGCACAAGGTCCCCCAGTAAAATGATATACATTGACACCA
GTGAAGATGCGGCCGTCGCTAGAGAGAGCTGCGCTGGCGACGCTGTAGTCTTCAGA
GATGGGGATGCTGTTGATTGTAGCCGTTGCTCTTTCAATGAGGGTGGATTCTTCTTG
AGACAAAGGCTTGGCCATGCGGCCGCCGCTCGGTGTTCGAGGCCACACGCGTCACC
TTAATATGCGAAGTGGACCTCGGACCGCGCCGCCCCGACTGCATCTGCGTGTTCGA
ATTCGCCAATGACAAGACGCTGGGCGGGGTTTGTGTCATCATAGAACTAAAGACAT
GCAAATATATTTCTTCCGGGGGGTACCGGCCTTTTTGGCCATTGGATCGGATCTGGC
CAAAAAGGCCCTTAAGTATTTACATTAAATGGCCATAGTACTTAAAGTTACATTGGC
TTCCTTGAAATAAACATGGAGTATTCAGAATGTGTCATAAATATTTCTAATTTTAAG
ATAGTATCTCCATTGGCTTTCTACTTTTTCTTTTATTTTTTTTTGTCCTCTGTCTTCCAT
TTGTTGTTGTTGTTGTTTGTTTGTTTGTTTGTTGGTTGGTTGGTTAATTTTTTTTTAAA
GATCCTACACTATAGTTCAAGCTAGACTATTAGCTACTCTGTAACCCAGGGTGACCT
TGAAGTCATGGGTAGCCTGCTGTTTTAGCCTTCCCACATCTAAGATTACAGGTATGA
GCTATCATTTTTGGTATATTGATTGATTGATTGATTGATGTGTGTGTGTGTGATTGTG
TTTGTGTGTGTGATTGTGTATATGTGTGTATGGTTGTGTGTGTGTGTGTGTGTGTGTG
TGTGTGTGTGTGTGTGTGTGTGTTGTGTATATATATTTATGGTAGTGAGAGGCAACG
CTCCGGCTCAGGTGTCAGGTTGGTTTTTGAGACAGAGTCTTTCACTTAGCTTGGAAT
TCACTGGCCGTCGTTTTACAACGTCGTGACTGGGAAAACCCTGGCGTTACCCAACTT
AATCGCCTTGCAGCACATCCCCCTTTCGCCAGCTGGCGTAATAGCGAAGAGGCCCG
CACCGATCGCCCTTCCCAACAGTTGCGCAGCCTGAATGGCGAATGGCGCCTGATGC
GGTATTTTCTCCTTACGCATCTGTGCGGTATTTCACACCGCATATGGTGCACTCTCAG
TACAATCTGCTCTGATGCCGCATAGTTAAGCCAGCCCCGACACCCGCCAACACCCG
CTGACGCGCCCTGACGGGCTTGTCTGCTCCCGGCATCCGCTTACAGACAAGCTGTGA
CCGTCTCCGGGAGCTGCATGTGTCAGAGGTTTTCACCGTCATCACCGAAACGCGCG
AGACGAAAGGGCCTCGTGATACGCCTATTTTTATAGGTTAATGTCATGATAATAATG
GTTTCTTAGACGTCAGGTGGCACTTTTCGGGGAAATGTGCGCGGAACCCCTATTTGT
TTATTTTTCTAAATACATTCAAATATGTATCCGCTCATGAGACAATAACCCTGATAA
ATGCTTCAATAATATTGAAAAAGGAAGAGTATGAGTATTCAACATTTCCGTGTCGCC
CTTATTCCCTTTTTTGCGGCATTTTGCCTTCCTGTTTTTGCTCACCCAGAAACGCTGG
TGAAAGTAAAAGATGCTGAAGATCAGTTGGGTGCACGAGTGGGTTACATCGAACTG
GATCTCAACAGCGGTAAGATCCTTGAGAGTTTTCGCCCCGAAGAACGTTTTCCAATG
ATGAGCACTTTTAAAGTTCTGCTATGTGGCGCGGTATTATCCCGTATTGACGCCGGG
CAAGAGCAACTCGGTCGCCGCATACACTATTCTCAGAATGACTTGGTTGAGTACTCA
CCAGTCACAGAAAAGCATCTTACGGATGGCATGACAGTAAGAGAATTATGCAGTGC
TGCCATAACCATGAGTGATAACACTGCGGCCAACTTACTTCTGACAACGATCGGAG
GACCGAAGGAGCTAACCGCTTTTTTGCACAACATGGGGGATCATGTAACTCGCCTT
GATCGTTGGGAACCGGAGCTGAATGAAGCCATACCAAACGACGAGCGTGACACCA
CGATGCCTGTAGCAATGGCAACAACGTTGCGCAAACTATTAACTGGCGAACTACTT
ACTCTAGCTTCCCGGCAACAATTAATAGACTGGATGGAGGCGGATAAAGTTGCAGG
ACCACTTCTGCGCTCGGCCCTTCCGGCTGGCTGGTTTATTGCTGATAAATCTGGAGC
CGGTGAGCGTGGGTCTCGCGGTATCATTGCAGCACTGGGGCCAGATGGTAAGCCCT
CCCGTATCGTAGTTATCTACACGACGGGGAGTCAGGCAACTATGGATGAACGAAAT
AGACAGATCGCTGAGATAGGTGCCTCACTGATTAAGCATTGGTAACTGTCAGACCA
AGTTTACTCATATATACTTTAGATTGATTTAAAACTTCATTTTTAATTTAAAAGGATC
TAGGTGAAGATCCTTTTTGATAATCTCATGACCAAAATCCCTTAACGTGAGTTTTCG
TTCCACTGAGCGTCAGACCCCGTAGAAAAGATCAAAGGATCTTCTTGAGATCCTTTT
TTTCTGCGCGTAATCTGCTGCTTGCAAACAAAAAAACCACCGCTACCAGCGGTGGTT
TGTTTGCCGGATCAAGAGCTACCAACTCTTTTTCCGAAGGTAACTGGCTTCAGCAGA
GCGCAGATACCAAATACTGTTCTTCTAGTGTAGCCGTAGTTAGGCCACCACTTCAAG
AACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGGA
TAAGGCGCAGCGGTCGGGCTGAACGGGGGGTTCGTGCACACAGCCCAGCTTGGAGC
GAACGACCTACACCGAACTGAGATACCTACAGCGTGAGCTATGAGAAAGCGCCACG
CTTCCCGAAGGGAGAAAGGCGGACAGGTATCCGGTAAGCGGCAGGGTCGGAACAG
GAGAGCGCACGAGGGAGCTTCCAGGGGGAAACGCCTGGTATCTTTATAGTCCTGTC
GGGTTTCGCCACCTCTGACTTGAGCGTCGATTTTTGTGATGCTCGTCAGGGGGGCGG
AGCCTATGGAAAAACGCCAGCAACGCGGCCTTTTTACGGTTCCTGGCCTTTTGCTGG
CCTTTTGCTCACATGTTCTTTCCTGCGTTATCCCCTGATTCTGTGGATAACCGTATTA
CCGCCTTTGAGTGAGCTGATACCGCTCGCCGCAGCCGAACGACCGAGCGCAGCGAG
TCAGTGAGCGAGGAAGCGGAAGAGCGCCCAATACGCAAACCGCCTCTCCCCGCGCG
TTGGCCGATTCATTAATGCAGCTGGCACGACAGGTTTCCCGACTGGAAAGCGGGCA
GTGAGCGCAACGCAATTAATGTGAGTTAGCTCACTCATTAGGCACCCCAGGCTTTAC
ACTTTATGCTTCCGGCTCGTATGTTGTGTGGAATTGTGAGCGGATAACAATTTCACA
CAGGAAACAGCTATGACCATGATTACGCCAAGCTTGGAATTCGAGCTTGCATGCCT
GCAGGTCGTTACATAACTTACGGTAAATGGCCCGCCTGGCTGACCGCCCAACGACC
CCCGCCCATTGACGTCAATAATGACGTATGTTCCCATAGTAACGCCAATAGGGACTT
TCCATTGACGTCAATGGGTGGAGTATTTACGGTAAACTGCCCACTTGGCAGTACATC
AAGTGTATCATATGCCAAGTACGCCCCCTATTGACGTCAATGACGGTAAATGGCCC
GCCTGGCATTATGCCCAGTACATGACCTTATGGGACTTTCCTACTTGGCAGTACATC
TACGTATTAGTCATCGCTATTACCATGGTGATGCGGTTTTGGCAGTACATCAATGGG
CGTGGATAGCGGTTTGACTCACGGGGATTTCCAAGTCTCCACCCCATTGACGTCAAT
GGGAGTTTGTTTTGGCACCAAAATCAACGGGACTTTCCAAAATGTCGTAACAACTCC
GCCCCATTGACGCAAATGGGCGGTAGGCGTGTACGGTGGGAGGTCTATATAAGCAG
AGCTCAATAAAAGAGCCCACAACCCCTCACTCGGCGCGCCAGTCCTCCGATTGACT
GAGTCGCCCGGGTACCCGTGTATCCAATAAACCCTCTTGCAGTTGCATCCGACTTGT
GGTCTCGCTGTTCCTTGGGAGGGTCTCCTCTGAGTGATTGACTACCCGTCAGCGGGG
GTCTTTCATTTGGGGGCTCGTCCGAGATCGGGAGACCCCTGCCCAGGGACCACCGA
CCCACCACCGGGAGGTAAGCTGGCCAGCAACTTATCTGTGTCTGTCCGATTGTCTAG
TGTCTATGACTGATTTTATGCGCCTGCGTCGGTACTAGTTAGCTAACTAGCTCTGTAT
CTGGCGGACCCGTGGTGGAACTGACGAGTTCGGAACACCCGGCCGCAACCCTGGGA
GACGTCCCAGGGACTTCGGGGGCCGTTTTTGTGGCCCGACCTGAGTCCTAAAATCCC
GATCGTTTAGGACTCTTTGGTGCACCCCCCTTAGAGGAGGGATATGTGGTTCTGGTA
GGAGACGAGAACCTAAAACAGTTCCCGCCTCCGTCTGAATTTTTGCTTTCGGTTTGG
GACCGAAGCCGCGCCGCGCGTCTTGTCTGCTGCAGCATCGTTCTGTGTTGTCTCTGT
CTGACTGTGTTTCTGTATTTGTCTGAAAATATGGGCCCCCCCTCGAGCTGTGGAATG
TGTGTCAGTTAGGGTGTGGAAAGTCCCCAGGCTCCCCAGCAGGCAGAAGTATGCAA
AGCATGCATCTCAATTAGTCAGCAACCAGGTGTGGAAAGTCCCCAGGCTCCCCAGC
AGGCAGAAGTATGCAAAGCATGCATCTCAATTAGTCAGCAACCATAGTCCCGCCCC
TAACTCCGCCCATCCCGCCCCTAACTCCGCCCAGTTCCGCCCATTCTCCGCCCCATG
GCTGACTAATTTTTTTTATTTATGCAGAGGCCGAGGCCGCCTCTGCCTCTGAGCTATT
CCAGAAGTAGTGAGGAGGCTTTTTTGGAGGCCTAGGCTTTTGCAAAAAGCTGGATC
CGCGTCAAGTGGAGCAAGGCAGGTGGACAGTCTCGAGGCCGCCACCATGGACTGG
ACTTGGATACTCTTTCTGGTCGCTGCCGCCACACGGGTGCACTCTAATTGGGTCAAC
GTGATCAGCGACCTGAAGAAGATCGAGGACCTGATCCAGAGCATGCACATCGACGC
CACACTGTACACCGAGTCCGATGTGCACCCTAGCTGCAAAGTGACCGCCATGAAGT
GCTTTCTGCTGGAACTGCAAGTGATCAGCCTGGAAAGCGGCGACGCCAGCATCCAC
GATACCGTGGAAAATCTGATCATCCTGGCCAACAACAGCCTGTCCAGCAACGGCAA
TGTGACCGAGAGCGGCTGCAAAGAGTGCGAGGAACTGGAAGAGAAGAACATCAAA
GAGTTTCTGCAGAGCTTCGTCCACATCGTGCAGATGTTCATCAACACCTCATCAGGC
GGCGGTGGTAGTGGAGGCGGAGGCTCAGGCGTGACCCCTGAGCCTATCTTCAGCCT
GATCGGCGGAGGTTCCGGAGGTGGCGGTTCCGGCGGAGGATCTCTTCAATTGCTGC
CTAGCTGGGCCATCACACTGATCTCCGTGAACGGCATCTTCGTGATCTGCTGCCTGA
CCTACTGCTTCGCCCCTAGATGCAGAGAGCGGAGAAGAAACGAGCGGCTGAGAAG
AGAAAGCGTGCGGCCTGTGGGTAGCGGCCAGTGTACCAACTACGCCCTGCTGAAAC
TGGCCGGCGACGTGGAATCTAATCCTGGACCTGGATCTGGCGAGGGACGCGGGAGT
CTACTGACGTGTGGAGACGTGGAGGAAAACCCTGGACCTATGGCTCTGCCCGTGAC
AGCTTTGCTGCTGCCTTTGGCACTGCTGCTGCATGCTGCTAGACCAGAGGTGCAGCT
GGTTGAATCTGGCGGAGGACTGGTTCAGCCTGGCGGATCTCTGAGACTGTCTTGTGC
CGCCAGCGGCTTCACCTTCAGCAGATACGATATGCACTGGGTCCGACAGGCCCCTG
GCAAAGGACTTGAATGGGTGTCCGTGATCTGGGGCAACGGCAACACACACTACCAC
AGCGCCCTGAAGTCCCGGTTCACCATCTCCAGAGACAACAGCAAGAACACCCTGTA
CCTGCAGATGAACAGCCTGAGAGCCGAGGACACCGCCGTGTACTACTGCACCCTGA
GAATCAAGGATTGGGGCCAGGGCACCATGGTCACCGTTTCTTCTGGAGGCGGAGGA
TCTGGTGGCGGAGGAAGTGGCGGAGGCGGTTCTGACGTGGTCATGACACAGAGCCC
TCTGAGCCTGCCTGTGACACTGGGACAGCCTGCCAGCATCAGCTGCAAGTCTAGCC
AGTCTCTGGTGGCCAGCGACGAGAACACCTACCTGAACTGGTTCCAGCAGAGGCCC
GGACAGTCTCCTAGACGGCTGATCTACCAGGTGTCCAAGCTGGATAGCGGCGTGCC
CGATAGATTTTCTGGCAGCGGCTCTGGCACCGACTTCACCCTGAAGATCAGCAGAG
TGGAAGCCGAGGACGTGGGCGTGTACTACTGTCTGCAAGGCATCCATCTGCCTTGG
ACCTTTGGCCAGGGCACAAAGCTGGAAATCAAGAATGGCGCTGCACACCCATCCGA
TCCTCTCGAGCTGGTGGTTTCTGGACCTTCTGGCGGCCCTAGCAGCCCTACAACAGG
ACCTACAAGCACAAGCGGCCCTGAGGACCAACCTCTGACACCAACAGGCAGCGATC
CTCAGTCTGGACTGGGGAGACATCTGGGCGTTGTGATCGGCATTCTGGTCGCCGTGA
TCCTGCTCCTGTTGCTCCTGCTGCTTCTGTTCCTGATCCTGCGGCACAGAAGGCAGG
GCAAGCACTGGACAAGCACCCAGAGAAAGGCCGACTTTCAGCATCCTGCTGGCGCC
GTTGGACCTGAGCCTACAGATAGAGGACTGCAGTGGCGGTCTAGCCCTGCCGCTGA
TGCCCAAGAGGAAAATCTTTACGCCGCCGTGAAGCACACCCAGCCTGAGGATGGCG
TGGAAATGGACACCAGATCTCCCCACGATGAGGACCCTCAGGCCGTGACATACGCA
GAAGTGAAGCACTCCAGACCTCGGAGAGAGATGGCAAGCCCTCCATCTCCTCTGAG
CGGCGAGTTCCTGGACACCAAAGACAGACAGGCCGAAGAGGACAGACAGATGGAT
ACCGAAGCCGCCGCTTCTGAAGCCCCACAGGATGTGACATATGCCCAGCTGCATAG
CCTGACACTGCGGAGAGAAGCCACAGAGCCTCCACCTTCTCAAGAAGGCCCATCTC
CTGCCGTGCCTTCCATCTATGCCACTCTGGCCATTCACGGTTCTGGCCAGTGCACAA
ATTATGCACTGCTGAAGCTCGCCGGGGATGTCGAGAGTAACCCAGGACCTGGAAGC
GGAGAAGGTCGTGGTAGTCTACTAACGTGTGGTGATGTAGAAGAAAATCCTGGACC
TATGCTGCTGCTGGTTACATCTCTGCTGCTGTGCGAGCTGCCCCATCCTGCCTTTCTG
CTTATTCCTGAAGTGCAGCTGGTTCAGTCTGGCGCCGAAGTGAAGAAACCTGGCAG
CAGCGTGAAGGTGTCCTGCAAAGCTTCTGGCGGCACCTTCAGCAGCTACGCCATCTC
TTGGGTTCGACAGGCCCCTGGACAAGGCCTGGAATGGATGGGAGGCATCATCCCCA
TCTTCGGCACCGCCAATTACGCCCAGAAATTCCAGGGCAGAGTGACCATCACCGCC
GACAAGAGCACAAGCACCGCCTACATGGAACTGAGCAGCCTGAGAAGCGAGGACA
CCGCCGTGTACTACTGCGCCACATTTGCCCTGTTCGGCTTCAGAGAGCAGGCCTTCG
ATATCTGGGGCCAGGGCACAACCGTGACCGTTTCTAGCGGAGGCGGAGGATCTGGT
GGTGGTGGATCTCAGGTTCAGCTGGTGCAGAGCGGAGCTGAAGTGAAAAAGCCAG
GCTCCTCCGTGAAAGTCTCTTGCAAGGCCAGCGGCTACACCTTTACCG

| [symbol] | | | [symbol] | [symbol] | [symbol] | [symbol] | [symbol] | [symbol] | [symbol] | | | | | | | [symbol] | [symbol] | [symbol] | [symbol] | [symbol] | [symbol] | [symbol] | | | [symbol] | [symbol] | [symbol] |
|---|---|---|---|---|---|---|---|---|---|---|---|---|---|---|---|---|---|---|---|---|---|---|---|---|---|---|---|
| [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |

[symbol]

indicates data missing or illegible when filed

### Interpretations

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A multicistronic expression system comprising an engineered nucleic acid comprising:
(A) an exogenous polynucleotide encoding a membrane-cleavable chimeric protein, oriented from N-terminal to C-terminal, having the formula:
S—C-MT or MT-C—S
wherein
S comprises a secretable effector molecule, wherein the secretable effector molecule comprises IL-15,
C comprises a protease cleavage site, and
MT comprises a cell membrane tethering domain,
wherein S—C-MT or MT-C—S is configured to be expressed as a single polypeptide;
(B) an exogenous polynucleotide encoding an inhibitory chimeric antigen receptor (iCAR) comprising:
(i) an antigen-binding domain specific for endomucin (EMCN);
(ii) one or more intracellular inhibitory domains that inhibit an immune response; and
(iii) one or more polypeptides selected from the group consisting of: a signal peptide, a transmembrane domain, a hinge domain, a spacer region, one or more peptide linkers, and combinations thereof; and
(C) an exogenous polynucleotide encoding a bivalent activating chimeric antigen receptor (aCAR) comprising:
(i) an antigen-binding domain specific for FLT3;
(ii) an antigen-binding domain specific for CD33;
(iii) one or more intracellular signaling domains that stimulate an immune response; and
(iv) one or more polypeptides selected from the group consisting of: a signal peptide, a transmembrane domain, a hinge domain, a spacer region, one or more peptide linkers, and combinations thereof.

2. The multicistronic expression system of claim 1, wherein the exogenous polynucleotides encoding each of the membrane-cleavable chimeric protein, the iCAR, and the bivalent aCAR are linked together by a polynucleotide linker encoding a 2A ribosome skipping element, optionally wherein the 2A ribosome skipping element is selected from the group consisting of: a T2A ribosome skipping element, a E2A ribosome skipping element, a P2A ribosome skipping element, a F2A ribosome skipping element, ribosome skipping element fusions thereof, and combinations thereof, optionally wherein the ribosome skipping element fusion comprises an E2A/T2A ribosome skipping element, optionally wherein the E2A/T2A ribosome skipping element comprises the amino acid sequence QCTNYALLKLAGDVESNPGPGSGEGRGSLLTCGDVEENPGP (SEQ ID NO: 221), optionally wherein the E2A/T2A ribosome skipping element is encoded by a polynucleotide sequence comprising the sequence CAGTGTACCAACTACGCCCTGCTGAAACTGGCCGGCGACGTGGAATCTAATC CTGGACCTGGATCTGGCGAGGGACGCGGGAGTCTACTGACGTGTGGAGACG TGGAGGAAAACCCTGGACCT (SEQ ID NO: 222) or the sequence CAGTGCACAAATTATGCACTGCTGAAGCTCGCCGGGGATGTCGAGAGTAACC CAGGACCTGGAAGCGGAGAAGGTCGTGGTAGTCTACTAACGTGTGGTGATGT AGAAGAAAATCCTGGACCT (SEQ ID NO: 223).

3. The multicistronic expression system of claim 1 or 2, wherein the membrane-cleavable chimeric protein, the iCAR, and the bivalent aCAR are encoded in order from 5′ to 3′: (i) membrane-cleavable chimeric protein; (ii) bivalent aCAR; and (iii) iCAR; or from 5′ to 3′: (i) membrane-cleavable chimeric protein; (ii) iCAR; and (iii) bivalent aCAR.

4. The multicistronic expression system of any one of claims 1-3, wherein the secretable effector molecule comprises a signal peptide or a signal-anchor sequence, optionally wherein the signal peptide comprises a native signal peptide native to the secretable effector molecule, optionally wherein the signal peptide comprises a non-native signal peptide or the signal-anchor sequence comprises a non-native signal-anchor sequence non-native to the secretable effector molecule, optionally wherein the non-native signal peptide or the non-native signal-anchor sequence is selected from the group consisting of: IgE, IL-12, IL-2, optimized IL-2, trypsiongen-2, Gaussia luciferase, CD5, human IgKVII, murine IgKVII, VSV-G, prolactin, serum albumin preprotein, azurocidin preprotein, osteonectin, CD33, IL-6, IL-8, CCL2, TIMP2, VEGFB, osteoprotegerin, serpin E1, GROalpha, CXCL12, IL-21, CD8, NKG2D, TNFR2, GMCSF, and GM-CSFRa, optionally wherein the non-native signal peptide comprises an IgE signal peptide, optionally wherein the IgE signal peptide comprises the amino acid sequence MDWTWILFLVAAATRVHS (SEQ ID NO: 228), optionally wherein the IgE signal peptide is encoded by a polynucleotide sequence comprising the sequence
(SEQ ID NO: 229)
ATGGACTGGACTTGGATACTCTTTCTGGTCGCTGCCGCCACACGGGTGC
ACTCT.

5. The multicistronic expression system of any one of claims 1-4, wherein the protease cleavage site further comprises the N-terminal peptide linker, optionally selected from the group consisting of: SGGGGSGGGGSG (SEQ ID NO: 230); GGGSGGGGSGGGSLQ (SEQ ID NO: 231); GGS (SEQ ID NO: 232); GGSGGS (SEQ ID NO: 233); GGSGGSGGS (SEQ ID NO: 234); GGSGGSGGSGGS (SEQ ID NO: 235; GGSGGSGGSGGSGGS (SEQ ID NO: 236); GGGS (SEQ ID NO: 237); GGGSGGGS (SEQ ID NO: 238); GGGSGGGSGGGS (SEQ ID NO: 239); GGGSGGGSGGGSGGGS (SEQ ID NO: 240); GGGSGGGSGGGSGGGSGGGS (SEQ ID NO: 241); GGGGS (SEQ ID NO: 242); GGGGSGGGGS (SEQ ID NO: 243); GGGGSGGGGSGGGGS (SEQ ID NO: 244); GGGGSGGGGSGGGGSGGGGS (SEQ ID NO: 245); GGGGSGGGGSGGGGSGGGGSGGGGS (SEQ ID NO: 246); GSTSGSGKPGSGEGSTKG (SEQ ID NO: 247); and EAAAKEAAAKEAAAKEAAAK (SEQ ID NO: 248), optionally wherein the protease cleavage site further comprises the N-terminal peptide linker SGGGGSGGGGSG (SEQ ID NO: 230), optionally wherein the protease cleavage site further comprises a C-terminal peptide linker, optionally selected from the group consisting of: SGGGGSGGGGSG (SEQ ID NO: 230); GGGSGGGGSGGGSLQ (SEQ ID NO: 231); GGS (SEQ ID NO: 232); GGSGGS (SEQ ID NO: 233); GGSGGSGGS (SEQ ID NO: 234); GGSGGSGGSGGS (SEQ ID NO: 235; GGSGGSGGSGGSGGS (SEQ ID NO: 236); GGGS (SEQ ID NO: 237); GGGSGGGS (SEQ ID NO: 238); GGGSGGGSGGGS (SEQ ID NO: 239); GGGSGGGSGGGSGGGS (SEQ ID NO: 240); GGGSGGGSGGGSGGGSGGGS (SEQ ID NO: 241); GGGGS (SEQ ID NO: 242); GGGGSGGGGS (SEQ ID NO: 243); GGGGSGGGGSGGGGS (SEQ ID NO: 244); GGGGSGGGGSGGGGSGGGGS (SEQ ID NO: 245); GGGGSGGGGSGGGGSGGGGSGGGGS (SEQ ID NO: 246); GSTSGSGKPGSGEGSTKG (SEQ ID NO: 247); and EAAAKEAAAKEAAAKEAAAK (SEQ ID NO: 248), optionally wherein the protease cleavage site further comprises the C-terminal linker GGGSGGGGSGGGSLQ (SEQ ID NO: 231), optionally wherein the protease cleavage site comprises a Tumor Necrosis Factor-α Converting Enzyme (TACE)-specific cleavage site, optionally wherein the TACE-specific cleavage site comprises the amino acid sequence VTPEPIFSLI (SEQ ID NO: 191), optionally, wherein the TACE-specific cleavage site comprises the amino acid sequence
(SEQ ID NO: 250)
SGGGGSGGGGSGVTPEPIFSLIGGGSGGGGSGGGSLQ,
optionally wherein the TACE-specific cleavage site is encoded by a polynucleotide sequence comprising the sequence
(SEQ ID NO: 251)
TCAGGCGGCGGTGGTAGTGGAGGCGGAGGCTCAGGCGTGACCCCTGAGCCT
ATCTTCAGCCTGATCGGCGGAGGTTCCGGAGGTGGCGGTTCCGGCGGAGGAT
CTCTTCAA

6. The multicistronic expression system of any one of claims 1-5, wherein the cell membrane tethering domain comprises a transmembrane domain selected from the group consisting of: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, LIR1, B7-1, and BTLA, optionally wherein the cell membrane tethering domain comprises a B7-1 transmembrane domain, optionally wherein the B7-1 transmembrane domain comprises the amino acid sequence LLPSWAITLISVNGIFVICCLTYCFAPRCRERRRNERLRRESVRPV (SEQ ID NO: 204, optionally wherein the B7-1 transmembrane domain is encoded by a polynucleotide sequence comprising the sequence TTGCTGCCTAGCTGGGCCATCACACTGATCTCCGTGAACGGCATCTTCGTGAT CTGCTGCCTGACCTACTGCTTCGCCCCTAGATGCAGAGAGCGGAGAAGAAAC GAGCGGCTGAGAAGAGAAAGCGTGCGGCCTGTG (SEQ ID NO: 252), optionally wherein the membrane-cleavable chimeric protein, oriented from N-terminal to C-terminal, has the formula S—C-MT, optionally wherein the membrane-cleavable chimeric protein comprises the amino acid sequence MDWTWILFLVAAATRVHSNWVNVISDLKKIEDLIQSMHIDATLYTESDVHPSCK VTAMKCFLLELQVISLESGDASIHDTVENLIILANNSLSSNGNVTESGCKECEELE EKNIKEFLQSFVHIVQMFINTSSGGGGSGGGGSGVTPEPIFSLIGGGSGGGGSGGG SLQLLPSWAITLISVNGIFVICCLTYCFAPRCRERRRNERLRRESVRPV (SEQ ID NO: 226), optionally wherein the membrane-cleavable chimeric protein is encoded by a polynucleotide sequence comprising the sequence
(SEQ ID NO: 227)
ATGGACTGGACTTGGATACTCTTTCTGGTCGCTGCCGCCACACGGGTGC
ACTCTAATTGGGTCAACGTGATCAGCGACCTGAAGAAGATCGAGGACCT
GATCCAGAGCATGCACATCGACGCCACACTGTACACCGAGTCCGATGTG
CACCCTAGCTGCAAAGTGACCGCCATGAAGTGCTTTCTGCTGGAACTGC
AAGTGATCAGCCTGGAAAGCGGCGACGCCAGCATCCACGATACCGTGGA
AAATCTGATCATCCTGGCCAACAACAGCCTGTCCAGCAACGGCAATGTG
ACCGAGAGCGGCTGCAAAGAGTGCGAGGAACTGGAAGAGAAGAACATCA
AAGAGTTTCTGCAGAGCTTCGTCCACATCGTGCAGATGTTCATCAACAC
CTCATCAGGCGGCGGTGGTAGTGGAGGCGGAGGCTCAGGCGTGACCCCT
GAGCCTATCTTCAGCCTGATCGGCGGAGGTTCCGGAGGTGGCGGTTCCG
GCGGAGGATCTCTTCAATTGCTGCCTAGCTGGGCCATCACACTGATCTC
CGTGAACGGCATCTTCGTGATCTGCTGCCTGACCTACTGCTTCGCCCCT
AGATGCAGAGAGCGGAGAAGAAACGAGCGGCTGAGAAGAGAAAGCGTGC
GGCCTGTG.

7. The multicistronic expression system of any one of claims 1-6, wherein the antigen-binding domain specific for EMCN comprises a heavy chain variable (VH) region and a light chain variable (VL) region, wherein:
the EMCN-VH comprises:
a heavy chain complementarity determining region 1 (CDR-H1) having the amino acid sequence of RYDMH (SEQ ID NO: 291),
a heavy chain complementarity determining region 2 (CDR-H2) having the amino acid sequence of VIWGNGNTHYHSALKS (SEQ ID NO: 296), and
a heavy chain complementarity determining region 3 (CDR-H3) having the amino acid sequence of RIKD (SEQ ID NO: 298), and
the EMCN-VL comprises:
a light chain complementarity determining region 1 (CDR-L1) having the amino acid sequence of KSSQSLVASDENTYLN (SEQ ID NO: 299),
a light chain complementarity determining region 2 (CDR-L2) having the amino acid sequence of QVSKLDS (SEQ ID NO: 300), and
a light chain complementarity determining region 3 (CDR-L3) having the amino acid sequence of LQGIHLPWT (SEQ ID NO: 301), and
wherein the amino acid sequences of the CDR-H1, the CDR-H2, the CDR-H3, the CDR-L1, the CDR-L2, and the CDR-L3 of the reference antibody are defined based on the Kabat numbering scheme; optionally wherein the EMCN-VH and the EMCN-VL are separated by a peptide linker, optionally wherein the antigen-binding domain specific for EMCN comprises the structure VH-L-VL or VL-L-VH, wherein L is the peptide linker, optionally wherein the peptide linker comprises the amino acid sequence SGGGGSGGGGSG (SEQ ID NO: 230); GGGSGGGGSGGGSLQ (SEQ ID NO: 231); GGS (SEQ ID NO: 232); GGSGGS (SEQ ID NO: 233); GGSGGSGGS (SEQ ID NO: 234; GGSGGSGGSGGS (SEQ ID NO: 235); GGSGGSGGSGGSGGS (SEQ ID NO: 236; GGGS (SEQ ID NO: 237); GGGSGGGS (SEQ ID NO: 238); GGGSGGGSGGGS (SEQ ID NO: 239); GGGSGGGSGGGSGGGS (SEQ ID NO: 240); GGGSGGGSGGGSGGGSGGGS (SEQ ID NO: 241); GGGGS (SEQ ID NO: 242); GGGGSGGGGS (SEQ ID NO: 243); GGGGSGGGGSGGGGS (SEQ ID NO: 244); GGGGSGGGGSGGGGSGGGGS (SEQ ID NO: 245); GGGGSGGGGSGGGGSGGGGSGGGGS (SEQ ID NO: 246); GSTSGSGKPGSGEGSTKG (SEQ ID NO: 247); or EAAAKEAAAKEAAAKEAAAK (SEQ ID NO: 248), optionally wherein the peptide linker comprises the amino acid sequence GGGGSGGGGSGGGGS (SEQ ID NO: 244), optionally wherein the peptide linker is encoded by a polynucleotide sequence comprising the sequence
(SEQ ID NO: 253)
GGAGGCGGAGGATCTGGTGGCGGAGGAAGTGGCGGAGGCGGTTCT.

8. The multicistronic expression system of any one of claims 1-7, wherein the intracellular inhibitory domain comprises a LIR1 intracellular inhibitory domain, optionally wherein the LIR 1 intracellular inhibitory domain comprises the amino acid sequence LRHRRQGKHWTSTQRKADFQHPAGAVGPEPTDRGLQWRSSPAADAQEENLYA AVKHTQPEDGVEMDTRSPHDEDPQAVTYAEVKHSRPRREMASPPSPLSGEFLDT KDRQAEEDRQMDTEAAASEAPQDVTYAQLHSLTLRREATEPPPSQEGPSPAVPSI YATLAIH (SEQ ID NO: 285), optionally wherein the LIR1 intracellular inhibitory domain is encoded by a polynucleotide sequence comprising the sequence
(SEQ ID NO: 286)
CTGCGGCACAGAAGGCAGGGCAAGCACTGGACAAGCACCCAGAGAAAGG
CCGACTTTCAGCATCCTGCTGGCGCCGTTGGACCTGAGCCTACAGATAG
AGGACTGCAGTGGCGGTCTAGCCCTGCCGCTGATGCCCAAGAGGAAAAT
CTTTACGCCGCCGTGAAGCACACCCAGCCTGAGGATGGCGTGGAAATGG
ACACCAGATCTCCCCACGATGAGGACCCTCAGGCCGTGACATACGCAGA
AGTGAAGCACTCCAGACCTCGGAGAGAGATGGCAAGCCCTCCATCTCCT
CTGAGCGGCGAGTTCCTGGACACCAAAGACAGACAGGCCGAAGAGGACA
GACAGATGGATACCGAAGCCGCCGCTTCTGAAGCCCCACAGGATGTGAC
ATATGCCCAGCTGCATAGCCTGACACTGCGGAGAGAAGCCACAGAGCCT
CCACCTTCTCAAGAAGGCCCATCTCCTGCCGTGCCTTCCATCTATGCCA
CTCTGGCCATTCAC.

9. The multicistronic expression system of any one of claims 1-8, wherein (A) the signal peptide is present in the iCAR, optionally wherein the signal peptide of the iCAR comprises a native signal peptide native or a non-native signal peptide, optionally wherein the non-native signal peptide or the non-native signal-anchor sequence is selected from the group consisting of: IgE, IL-12, IL-2, optimized IL-2, trypsiongen-2, Gaussia luciferase, CD5, human IgKVII, murine IgKVII, VSV-G, prolactin, serum albumin preprotein, azurocidin preprotein, osteonectin, CD33, IL-6, IL-8, CCL2, TIMP2, VEGFB, osteoprotegerin, serpin E1, GROalpha, CXCL12, IL-21, CD8, NKG2D, TNFR2, and GMCSF, optionally wherein the signal peptide of the iCAR comprises a CD8 signal peptide, optionally wherein the CD8 signal peptide comprises the amino acid sequence MALPVTALLLPLALLLHAARP (SEQ ID NO: 137), optionally wherein the CD8 signal peptide is encoded by a polynucleotide sequence comprising the sequence ATGGCTCTGCCCGTGACAGCTTTGCTGCTGCCTTTGGCACTGCTGCTGCATGC TGCTAGACCA (SEQ ID NO: 416); and/or wherein (B) the hinge domain is present in the iCAR comprises, optionally wherein the hinge domain of the iCAR is selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof, optionally wherein the hinge domain of the iCAR comprises a LIR1 hinge, optionally wherein the LIR1 hinge comprises the amino acid sequence HPSDPLELVVSGPSGGPSSPTTGPTSTSGPEDQPLTPTGSDPQSGLGRHLGV (SEQ ID NO: 417), optionally wherein the LIR1 hinge comprises is encoded by a polynucleotide sequence comprising the sequence CACCCATCCGATCCTCTCGAGCTGGTGGTTTCTGGACCTTCTGGCGGCCCTAG CAGCCCTACAACAGGACCTACAAGCACAAGCGGCCCTGAGGACCAACCTCT GACACCAACAGGCAGCGATCCTCAGTCTGGACTGGGGAGACATCTGGGCGTT (SEQ ID NO: 418); optionally wherein the hinge domain of the iCAR comprises a CD8 hinge, optionally wherein the CD8 hinge comprises the amino acid sequence TTTPAPRPPTPAPTIALQPLSLRPEACRPAAGGAVHTRGLDFACD (SEQ ID NO: 271, optionally wherein the CD8 hinge comprises is encoded by a polynucleotide sequence comprising the sequence ACAACAACACCCGCACCTCGGCCTCCAACTCCAGCTCCAACAATTGCACTGC AACCCCTGAGTCTGAGGCCCGAGGCCTGTAGGCCAGCAGCTGGCGGAGCTGT TCACACTAGAGGCCTGGACTTTGCCTGTGAC (SEQ ID NO: 283); and/or wherein (C) the transmembrane domain of the iCAR is present, optionally wherein the transmembrane domain of the iCAR comprises a transmembrane domain selected from the group consisting of: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, LIR1, and BTLA, optionally wherein the transmembrane domain of the iCAR comprises a LIR1 transmembrane domain, optionally wherein the LIR1 transmembrane domain comprises the amino acid sequence VIGILVAVILLLLLLLLLFLI (SEQ ID NO: 259), optionally wherein the LIR1 transmembrane domain is encoded by a polynucleotide sequence comprising the sequence
(SEQ ID NO: 260)
GTGATCGGCATTCTGGTCGCCGTGATCCTGCTCCTGTTGCTCCTGCTGCT
TCTGTTCCTGATC.

10. The multicistronic expression system of any one of claims 1-9, wherein the iCAR comprises the amino acid sequence:
MALPVTALLLPLALLLHAARPEVQLVESGGGLVQPGGSLRLSCAASGFTFSRYD MHWVRQAPGKGLEWVSVIWGNGNTHYHSALKSRFTISRDNSKNTLYLQMNSL RAEDTAVYYCTLRIKDWGQGTMVTVSSGGGGSGGGGSGGGGSDVVMTQSPLS LPVTLGQPASISCKSSQSLVASDENTYLNWFQQRPGQSPRRLIYQVSKLDSGVPD RFSGSGSGTDFTLKISRVEAEDVGVYYCLQGIHLPWTFGQGTKLEIKngaaHPSDP LELVVSGPSGGPSSPTTGPTSTSGPEDQPLTPTGSDPQSGLGRHLGVVIGILVAVIL LLLLLLLLFLILRHRRQGKHWTSTQRKADFQHPAGAVGPEPTDRGLQWRSSPAA DAQEENLYAAVKHTQPEDGVEMDTRSPHDEDPQAVTYAEVKHSRPRREMASPP SPLSGEFLDTKDRQAEEDRQMDTEAAASEAPQDVTYAQLHSLTLRREATEPPPS QEGPSPAVPSIYATLAIH (SEQ ID NO: 419), optionally wherein the iCAR is encoded by a polynucleotide sequence comprising the sequence
(SEQ ID NO: 420)
ATGGCTCTGCCCGTGACAGCTTTGCTGCTGCCTTTGGCACTGCTGCTGCA
TGCTGCTAGACCAGAGGTGCAGCTGGTTGAATCTGGCGGAGGACTGGTTC
AGCCTGGCGGATCTCTGAGACTGTCTTGTGCCGCCAGCGGCTTCACCTTC
AGCAGATACGATATGCACTGGGTCCGACAGGCCCCTGGCAAAGGACTTGA
ATGGGTGTCCGTGATCTGGGGCAACGGCAACACACACTACCACAGCGCCC
TGAAGTCCCGGTTCACCATCTCCAGAGACAACAGCAAGAACACCCTGTAC
CTGCAGATGAACAGCCTGAGAGCCGAGGACACCGCCGTGTACTACTGCAC
CCTGAGAATCAAGGATTGGGGCCAGGGCACCATGGTCACCGTTTCTTCTG
GAGGCGGAGGATCTGGTGGCGGAGGAAGTGGCGGAGGCGGTTCTGACGTG
GTCATGACACAGAGCCCTCTGAGCCTGCCTGTGACACTGGGACAGCCTGC
CAGCATCAGCTGCAAGTCTAGCCAGTCTCTGGTGGCCAGCGACGAGAACA
CCTACCTGAACTGGTTCCAGCAGAGGCCCGGACAGTCTCCTAGACGGCTG
ATCTACCAGGTGTCCAAGCTGGATAGCGGCGTGCCCGATAGATTTTCTGG
CAGCGGCTCTGGCACCGACTTCACCCTGAAGATCAGCAGAGTGGAAGCCG
AGGACGTGGGCGTGTACTACTGTCTGCAAGGCATCCATCTGCCTTGGACC
TTTGGCCAGGGCACAAAGCTGGAAATCAAGAATGGCGCTGCACACCCATC
CGATCCTCTCGAGCTGGTGGTTTCTGGACCTTCTGGCGGCCCTAGCAGCC
CTACAACAGGACCTACAAGCACAAGCGGCCCTGAGGACCAACCTCTGACA
CCAACAGGCAGCGATCCTCAGTCTGGACTGGGGAGACATCTGGGCGTTGT
GATCGGCATTCTGGTCGCCGTGATCCTGCTCCTGTTGCTCCTGCTGCTTC
TGTTCCTGATCCTGCGGCACAGAAGGCAGGGCAAGCACTGGACAAGCACC
CAGAGAAAGGCCGACTTTCAGCATCCTGCTGGCGCCGTTGGACCTGAGCC
TACAGATAGAGGACTGCAGTGGCGGTCTAGCCCTGCCGCTGATGCCCAAG
AGGAAAATCTTTACGCCGCCGTGAAGCACACCCAGCCTGAGGATGGCGTG
GAAATGGACACCAGATCTCCCCACGATGAGGACCCTCAGGCCGTGACATA
CGCAGAAGTGAAGCACTCCAGACCTCGGAGAGAGATGGCAAGCCCTCCAT
CTCCTCTGAGCGGCGAGTTCCTGGACACCAAAGACAGACAGGCCGAAGAG
GACAGACAGATGGATACCGAAGCCGCCGCTTCTGAAGCCCCACAGGATGT
GACATATGCCCAGCTGCATAGCCTGACACTGCGGAGAGAAGCCACAGAGC
CTCCACCTTCTCAAGAAGGCCCATCTCCTGCCGTGCCTTCCATCTATGCC
ACTCTGGCCATTCAC.

11. The multicistronic expression system of any one of claims 1-10, wherein the antigen-binding domain specific for FLT3 comprises a heavy chain variable (VH) region and a light chain variable (VL) region, wherein:
the FLT3-VH comprises:
a heavy chain complementarity determining region 1 (CDR-H1) having the amino acid sequence of GGTFSSYAIS (SEQ ID NO: 360),
a heavy chain complementarity determining region 2 (CDR-H2) having the amino acid sequence of GIIPIFGTANYAQKFQG (SEQ ID NO: 361), and
a heavy chain complementarity determining region 3 (CDR-H3) having the amino acid sequence of FALFGFREQAFDI (SEQ ID NO: 362), and
the FLT3-VL comprises:
a light chain complementarity determining region 1 (CDR-L1) having the amino acid sequence of RASQSISSYLN (SEQ ID NO: 363),
a light chain complementarity determining region 2 (CDR-L2) having the amino acid sequence of AASSLQS (SEQ ID NO: 364), and
a light chain complementarity determining region 3 (CDR-L3) having the amino acid sequence of QQSYSTPFT (SEQ ID NO: 365), and
wherein the amino acid sequences of the CDR-H1, the CDR-H2, the CDR-H3, the CDR-L1, the CDR-L2, and the CDR-L3 of the reference antibody are defined based on the Kabat numbering scheme; optionally wherein:
the FLT3-VH comprises the amino acid sequence EVQLVQSGAEVKKPGSSVKVSCKASGGTFSSYAISWVRQAPGQGLEWMGGIIPI FGTANYAQKFQGRVTITADKSTSTAYMELSSLRSEDTAVYYCATFALFGFREQA FDIWGQGTTVTVSS (SEQ ID NO: 315); and
the FLT3-VL comprises the amino acid sequence DIQMTQSPSSLSASVGDRVTITCRASQSISSYLNWYQQKPGKAPKLLIYAASSLQS GVPSRFSGSGSGTDFTLTISSLQPEDLATYYCQQSYSTPFTFGPGTKVDIK (SEQ ID NO: 316), optionally wherein the FLT3-VH, and the FLT3-VL are separated by peptide linkers.

12. The multicistronic expression system of any one of claims 1-11, wherein the antigen-binding domain specific for CD33 comprises a heavy chain variable (VH) region and a light chain variable (VL) region, wherein:
the CD33-VH comprises:
a heavy chain complementarity determining region 1 (CDR-H1) having the amino acid sequence of DYNMH (SEQ ID NO: 402),
a heavy chain complementarity determining region 2 (CDR-H2) having the amino acid sequence of YIYPYNGGTGYNQKFKSKA (SEQ ID NO: 403), and
a heavy chain complementarity determining region 3 (CDR-H3) having the amino acid sequence of GRPAMDYWGQ (SEQ ID NO: 404), and
the CD33-VL comprises:
a light chain complementarity determining region 1 (CDR-L1) having the amino acid sequence of RASESVDNYGISFMN (SEQ ID NO: 405),
a light chain complementarity determining region 2 (CDR-L2) having the amino acid sequence of AASNQGS (SEQ ID NO: 406), and
a light chain complementarity determining region 3 (CDR-L3) having the amino acid sequence of QQSKEVPWT (SEQ ID NO: 407), and
wherein the amino acid sequences of the CDR-H1, the CDR-H2, the CDR-H3, the CDR-L1, the CDR-L2, and the CDR-L3 of the reference antibody are defined based on the Kabat numbering scheme; optionally wherein:
the CD33-VH comprises the amino acid sequence QVQLVQSGAEVKKPGSSVKVSCKASGYTFTDYNMHWVRQAPGQGLEWIGYIY PYNGGTGYNQKFKSKATITADESTNTAYMELSSLRSEDTAVYYCARGRPAMDY WGQGTLVTVSS (SEQ ID NO: 329); and
the CD33-VL comprises the amino acid sequence DIQMTQSPSSLSASVGDRVTITCRASESVDNYGISFMNWFQQKPGKAPKLLIYAA SNQGSGVPSRFSGSGSGTDFTLTISSLQPDDFATYYCQQSKEVPWTFGQGTKVEI K (SEQ ID NO: 330), optionally wherein the CD33-VH, and the CD33-VL are separated by peptide linkers.

13. The multicistronic expression system of any one of claims 1-12, wherein the aCAR antigen binding domains comprises the structure (FLT3-VH)-L1-(CD33-VH)-L2-(CD33-VL)-L3-(FLT3-VL), wherein L1, L2, and L3 are a first, a second, and a third peptide linker, respectively; optionally wherein the L1, L2, and/or L3 are each independently selected from the group consisting of: SEQ ID Nos 230-248, optionally wherein the L1 peptide linker is the amino acid sequence GGGGS (SEQ ID NO: 242) or GGGGSGGGGS (SEQ ID NO: 243), optionally wherein the L1 peptide linker GGGGS (SEQ ID NO: 242) is encoded by a polynucleotide sequence comprising the sequence GGCGGCGGTGGCTCT (SEQ ID NO: 254) or the L1 peptide linker GGGGSGGGGS (SEQ ID NO: 243) is encoded by a polynucleotide sequence comprising the sequence GGAGGCGGAGGATCTGGTGGTGGTGGATCT (SEQ ID NO: 256), optionally wherein the L2 peptide linker is the amino acid sequence GSTSGSGKPGSGEGSTKG (SEQ ID NO: 247), optionally wherein the L2 peptide linker is encoded by a polynucleotide sequence comprising the sequence GGCTCTACATCTGGCTCTGGCAAACCTGGAAGCGGCGAGGGATCTACCAAGG GC (SEQ ID NO: 249), optionally wherein the L2 peptide linker is the amino acid sequence GGGGSGGGGS (SEQ ID NO: 243), optionally wherein the L2 peptide linker is encoded by a polynucleotide sequence comprising the sequence GGTGGCGGAGGAAGTGGCGGCGGAGGCTCT (SEQ ID NO: 257), optionally wherein the L3 peptide linker is the amino acid sequence GGGGS (SEQ ID NO: 242) or GGGGSGGGGS (SEQ ID NO: 243), optionally wherein the L3 peptide linker GGGGS (SEQ ID NO: 242) is encoded by a polynucleotide sequence comprising the sequence GGTGGCGGCGGATCC (SEQ ID NO: 255) or the L3 peptide linker GGGGSGGGGS (SEQ ID NO: 243) is encoded by a polynucleotide sequence comprising the sequence GGCGGTGGCGGATCTGGCGGAGGTGGCAGT (SEQ ID NO: 258).

14. The multicistronic expression system of any one of claims 1-13, wherein the aCAR intracellular signaling domains that stimulate an immune response is selected from the group consisting of: CD3-zeta, FcR gamma, FcR beta, CD3 gamma, CD3 delta, CD3 epsilon, CD5, CD22, CD79a, CD79b, CD278, FcεRI, DAP10, DAP12, CD66d, CD97, CD2, ICOS, CD27, CD154, CD8, OX40, 4-1BB, CD28, ZAP40, CD30, GITR, HVEM, DAP10, DAP12, MyD88, 2B4, CD40, PD-1, LFA-1, CD7, LIGHT, NKG2C, B7-H3, an MHC class I molecule, a TNF receptor protein, an Immunoglobulin-like protein, a cytokine receptor, an integrin, a SLAM protein, an activating NK cell receptor, BTLA, a Toll ligand receptor, CDS, ICAM-1, (CD11a/CD18), BAFFR, KIRDS2, SLAMF7, NKp80 (KLRF1), NKp44, NKp30, NKp46, CD19, CD4, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and combinations thereof, optionally wherein the aCAR intracellular signaling domains that stimulate an immune response comprise a CD28 co-stimulatory domain and a CD35 signaling domain, optionally wherein the CD28 co-stimulatory domain comprises the amino acid sequence RSKRSRLLHSDYMNMTPRRPGPTRKHYQPYAPPRDFAAYRS (SEQ ID NO: 287), optionally wherein the CD28 co-stimulatory domain is encoded by a polynucleotide sequence comprising the sequence AGAAGCAAGCGGAGCAGACTGCTGCACAGCGACTACATGAACATGACCCCT AGACGGCCCGGACCTACCAGAAAGCACTACCAGCCTTACGCTCCTCCTAGAG ATTTCGCCGCCTACCGGTCC (SEQ ID NO: 288), optionally wherein the CD35 signaling domain comprises the amino acid sequence RVKFSRSADAPAYKQGQNQLYNELNLGRREEYDVLDKRRGRDPEMGGKPRRK NPQEGLYNELQKDKMAEAYSEIGMKGERRRGKGHDGLYQGLSTATKDTYDAL HMQALPPR (SEQ ID NO: 289), optionally wherein the CD3 signaling domain is encoded by a polynucleotide sequence comprising the sequence AGAGTGAAGTTCAGCAGGAGCGCAGACGCCCCCGCGTACAAGCAGGGCCAG AACCAGCTCTATAACGAGCTCAATCTAGGACGAAGAGAGGAGTACGATGTTT TGGACAAGAGACGTGGCCGGGACCCTGAGATGGGGGGAAAGCCGAGAAGG AAGAACCCTCAGGAAGGCCTGTACAATGAACTGCAGAAAGATAAGATGGCG GAGGCCTACAGTGAGATTGGGATGAAAGGCGAGCGCCGGAGGGGCAAGGG GCACGATGGCCTTTACCAGGGTCTCAGTACAGCCACCAAGGACACCTACGAC GCCCTTCACATGCAGGCCCTGCCCCCTCGC (SEQ ID NO: 290), optionally wherein
(A) the hinge domain of the aCAR is present, optionally wherein the hinge domain of the aCAR is selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof, optionally wherein the hinge domain of the aCAR comprises a CD8 hinge, optionally wherein the CD8 hinge comprises the amino acid sequence ALSNSIMYFSHFVPVFLPAKPTTTPAPRPPTPAPTIASQPLSLRPEACRPAAGGAV HTRGLDFACD (SEQ ID NO: 272), optionally wherein the CD8 hinge comprises is encoded by a polynucleotide sequence comprising the sequence GCCCTGAGCAACAGCATCATGTACTTCAGCCACTTCGTGCCCGTGTTTCTGCC CGCCAAGCCTACAACAACCCCTGCTCCTAGACCACCTACACCAGCTCCTACA ATCGCCAGCCAGCCTCTGTCTCTGAGGCCCGAAGCTTGTAGACCAGCTGCTG GCGGAGCCGTGCATACAAGAGGACTGGATTTTGCCTGCGAC (SEQ ID NO: 284, and/or wherein
(B) the transmembrane domain of the aCAR is present, optionally wherein the transmembrane domain of the aCAR is selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof, optionally wherein the transmembrane domain of the aCAR comprises a CD8 hinge, optionally wherein the CD8 transmembrane comprises the amino acid sequence IYIWAPLAGTCGVLLLSLVITLYCNHR (SEQ ID NO: 206), optionally wherein the CD8 transmembrane comprises is encoded by a polynucleotide sequence comprising the sequence ATCTATATCTGGGCCCCTCTGGCTGGCACATGCGGAGTTCTGCTGCTCAGCCT GGTCATCACCCTGTACTGCAACCACAGA (SEQ ID NO: 261); and/or wherein
(C) the aCAR signal peptide of the aCAR is present, optionally wherein the signal peptide of the aCAR is selected from the group consisting of: IgE, IL-12, IL-2, optimized IL-2, trypsiongen-2, Gaussia luciferase, CD5, human IgKVII, murine IgKVII, VSV-G, prolactin, serum albumin preprotein, azurocidin preprotein, osteonectin, CD33, IL-6, IL-8, CCL2, TIMP2, VEGFB, osteoprotegerin, serpin E1, GROalpha, CXCL12, IL-21, CD8, NKG2D, TNFR2, GMCSF, and GM-CSFRa, optionally wherein the signal peptide of the aCAR comprises a GM-CSFRa signal peptide, optionally wherein the GM-CSFRa signal peptide comprises the amino acid sequence MLLLVTSLLLCELPHPAFLLIP (SEQ ID NO: 423), optionally wherein the GM-CSFRa signal peptide is encoded by a polynucleotide sequence comprising the sequence ATGCTGCTGCTGGTTACATCTCTGCTGCTGTGCGAGCTGCCCCATCCTGCCTT TCTGCTTATTCCT (SEQ ID NO: 424), optionally wherein the aCAR comprises the amino acid sequence MLLLVTSLLLCELPHPAFLLIPEVQLVQSGAEVKKPGSSVKVSCKASGGTFSSYAI SWVRQAPGQGLEWMGGIIPIFGTANYAQKFQGRVTITADKSTSTAYMELSSLRS EDTAVYYCATFALFGFREQAFDIWGQGTTVTVSSGGGGSQVQLVQSGAEVKKP GSSVKVSCKASGYTFTDYNMHWVRQAPGQGLEWIGYIYPYNGGTGYNQKFKS KATITADESTNTAYMELSSLRSEDTAVYYCARGRPAMDYWGQGTLVTVSSGST SGSGKPGSGEGSTKGDIQMTQSPSSLSASVGDRVTITCRASESVDNYGISFMNWF QQKPGKAPKLLIYAASNQGSGVPSRFSGSGSGTDFTLTISSLQPDDFATYYCQQS KEVPWTFGQGTKVEIKGGGGSDIQMTQSPSSLSASVGDRVTITCRASQSISSYLN WYQQKPGKAPKLLIYAASSLQSGVPSRFSGSGSGTDFTLTISSLQPEDLATYYCQ QSYSTPFTFGPGTKVDIKALSNSIMYFSHFVPVFLPAKPTTTPAPRPPTPAPTIASQ PLSLRPEACRPAAGGAVHTRGLDFACDIYIWAPLAGTCGVLLLSLVITLYCNHRR SKRSRLLHSDYMNMTPRRPGPTRKHYQPYAPPRDFAAYRSRVKFSRSADAPAY KQGQNQLYNELNLGRREEYDVLDKRRGRDPEMGGKPRRKNPQEGLYNELQKD KMAEAYSEIGMKGERRRGKGHDGLYQGLSTATKDTYDALHMQALPPR (SEQ ID NO: 414), optionally wherein the aCAR comprises the amino acid sequence
(SEQ ID NO: 415)
MLLLVTSLLLCELPHPAFLLIPEVQLVQSGAEVKKPGSSVKVSCKASGGT
FSSYAISWVRQAPGQGLEWMGGIIPIFGTANYAQKFQGRVTITADKSTST
AYMELSSLRSEDTAVYYCATFALFGFREQAFDIWGQGTTVTVSSGGGGSG
GGGSQVQLVQSGAEVKKPGSSVKVSCKASGYTFTDYNMHWVRQAPGQGLE
WIGYIYPYNGGTGYNQKFKSKATITADESTNTAYMELSSLRSEDTAVYYC
ARGRPAMDYWGQGTLVTVSSGGGGSGGGGSDIQMTQSPSSLSASVGDRVT
ITCRASESVDNYGISFMNWFQQKPGKAPKLLIYAASNQGSGVPSRFSGSG
SGTDFTLTISSLQPDDFATYYCQQSKEVPWTFGQGTKVEIKGGGGSGGGG
SDIQMTQSPSSLSASVGDRVTITCRASQSISSYLNWYQQKPGKAPKLLIY
AASSLQSGVPSRFSGSGSGTDFTLTISSLQPEDLATYYCQQSYSTPFTFG
PGTKVDIKALSNSIMYFSHFVPVFLPAKPTTTPAPRPPTPAPTIASQPLS
LRPEACRPAAGGAVHTRGLDFACDIYIWAPLAGTCGVLLLSLVITLYCNH
RRSKRSRLLHSDYMNMTPRRPGPTRKHYQPYAPPRDFAAYRSRVKFSRSA
DAPAYKQGQNQLYNELNLGRREEYDVLDKRRGRDPEMGGKPRRKNPQEGL
YNELQKDKMAEAYSEIGMKGERRRGKGHDGLYQGLSTATKDTYDALHMQA
LPPR.

15. An expression vector comprising the multicistronic expression system of any one of claims 1-14, optionally wherein the expression vector comprises the polynucleotide of SEQ ID NO: 425, SEQ ID NO: 426, SEQ ID NO: 427, SEQ ID NO: 428, SEQ ID NO: 429, SEQ ID NO: 433, or SEQ ID NO: 432.

16. An isolated cell comprising the multicistronic expression system of any one of claims 1-14 or the expression vector of claim 15, optionally wherein the cell comprises an immune cell, optionally wherein the cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell, optionally wherein the cell is an NK cell.

17. A polynucleotide comprising the nucleic acid sequence selected from the group consisting of SEQ ID NO: 425, SEQ ID NO: 426, SEQ ID NO: 427, SEQ ID NO: 428, SEQ ID NO: 429, SEQ ID NO: 433, SEQ ID NO: 432, and SEQ ID NO: 433.

18. A method of treating a subject in need thereof, the method comprising administering a therapeutically effective dose of any of the isolated cells of claim 16 or the polynucleotide of any one of claim 17, optionally wherein the subject has cancer, optionally wherein the isolated cell is derived from the subject or is allogeneic with reference to the subject.

19. A method of stimulating a cell-mediated immune response to a tumor cell in a subject, the method comprising administering to a subject having a tumor a therapeutically effective dose of any of the isolated cells of claim 16 or the polynucleotide claim 17, optionally wherein the isolated cell is derived from the subject or is allogeneic with reference to the subject.

20. A method of making an engineered cell, comprising transducing an isolated cell with the multicistronic expression system of any one of claims 1-14, the expression vector of claim 15, or the polynucleotide of claim 17.


SEQUENCE LISTING (527 sequences)

SEQ ID NO: 1 - DNA, 31 bp, synthetic construct
  source 1..31: mol_type = other DNA
agagggtatataatggaagctcgacttccag

SEQ ID NO: 2 - DNA, 52 bp, synthetic construct
  source 1..52: mol_type = other DNA
gggaatttccggggactttccgggaatttccggggactttccgggaatttcc

SEQ ID NO: 3 - DNA, 85 bp, synthetic construct
  source 1..85: mol_type = other DNA
caccagacagtgacgtcagctgccagatcccatggccgtcatactgtgacgtctttcagacaccccattgacgtcaatgggagaa

SEQ ID NO: 4 - DNA, 90 bp, synthetic construct
  source 1..90: mol_type = other DNA
ggaggaaaaactgtttcatacagaaggcgtggaggaaaaactgtttcatacagaaggcgtggaggaaaaactgtttcatacagaaggcgt

SEQ ID NO: 5 - DNA, 115 bp, synthetic construct
  source 1..115: mol_type = other DNA
aggatgtccatattaggacatctaggatgtccatattaggacatctaggatgtccatattaggacatctaggatgtccatattaggacatctaggatgtccatattaggacatct

SEQ ID NO: 6 - DNA, 115 bp, synthetic construct
  source 1..115: mol_type = other DNA
agtatgtccatattaggacatctaccatgtccatattaggacatctactatgtccatattaggacatcttgtatgtccatattaggacatctaaaatgtccatattaggacatct

SEQ ID NO: 7 - DNA, 48 bp, synthetic construct
  source 1..48: mol_type = other DNA
tgagtcagtgactcagtgagtcagtgactcagtgagtcagtgactcag

SEQ ID NO: 8 - DNA, 120 bp, synthetic construct
  source 1..120: mol_type = other DNA
agatcaaagggtttaagatcaaagggcttaagatcaaagggtataagatcaaagggcctaagatcaaagggactaagatcaaagggtttaagatcaaagggcttaagatcaaagggccta

SEQ ID NO: 9 - DNA, 32 bp, synthetic construct
  source 1..32: mol_type = other DNA
gtctagacgtctagacgtctagacgtctagac

SEQ ID NO: 10 - DNA, 24 bp, synthetic construct
  source 1..24: mol_type = other DNA
cagacacagacacagacacagaca

SEQ ID NO: 11 - DNA, 65 bp, synthetic construct
  source 1..65: mol_type = other DNA
ggatccggtactcgagatctgcgatctaagtaagcttggcattccggtactgttggtaaagccac

SEQ ID NO: 12 - DNA, 588 bp, unidentified
  source 1..588: mol_type = other DNA; note = Cytomegalovirus
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 13 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgccgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcgaccaccgagaatcggacgggggtagtctcaagctggccggcctgctctggtgcctgtcctcgcgccgccgtgtatcgccccgccccgggcggcaaggctggcccggtcggcaccagttgcgtgagcggaaagatggccgcttcccggtcctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 14 - DNA, 544 bp, synthetic construct
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 15 - DNA, 399 bp, synthetic construct
  source 1..399: mol_type = other DNA
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 16 - DNA, 511 bp, synthetic construct
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 17 - DNA, 408 bp, unidentified
  source 1..408: mol_type = other DNA; note = Spleen focus-forming virus
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 18 - DNA, 344 bp, unidentified
  source 1..344: mol_type = other DNA; note = Simian virus 40
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 19 - DNA, 1229 bp, synthetic construct
  source 1..1229: mol_type = other DNA
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagac

SEQ ID NO: 20 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 21 - DNA, 1718 bp, synthetic construct
  source 1..1718: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 22 - DNA, 212 bp, synthetic construct
  source 1..212: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 23 - DNA, 2379 bp, synthetic construct
  source 1..2379: mol_type = other DNA
ccactagttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctat

SEQ ID NO: 24 - DNA, 529 bp, synthetic construct
  source 1..529: mol_type = other DNA
gttgatttccttcatccctggcacacgtccaggcagtgtcgaatccatctctgctacaggggaaaacaaataacatttgagtccagtggagaccgggagcagaagtaaagggaagtgataacccccagagcccggaagcctctggaggctgagacctcgccccccttgcgtgatagggcctacggagccacatgaccaaggcactgtcgcctccgcacgtgtgagagtgcagggccccaagatggctgccaggcctcgaggcctgactcttctatgtcacttccgtaccggcgagaaaggcgggccctccagccaatgaggctgcggggcgggccttcaccttgataggcactcgagttatccaatggtgcctgcgggccggagcgactaggaactaacgtcatgccgagttgctgagcgccggcaggcggggccggggcggccaaaccaatgcgatggccggggcggagtcgggcgctctataagttgtcgataggcgggcactccgccctagtttctaaggaccatg

SEQ ID NO: 25 - DNA, 794 bp, synthetic construct
  source 1..794: mol_type = other DNA
agttccccaactttcccgcctctcagcctttgaaagaaagaaaggggagggggcaggccgcgtgcagtcgcgagcggtgctgggctccggctccaattccccatctcagtcgctcccaaagtccttctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgggtgtcggccggggccactaggcgctcactgttctctccctccgcgcagccgagccacatcgctgagacac

SEQ ID NO: 26 - DNA, 532 bp, synthetic construct
  source 1..532: mol_type = other DNA
agtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcac

SEQ ID NO: 27 - DNA, 701 bp, synthetic construct
  source 1..701: mol_type = other DNA
tagtttcatcaccaccgccacccccccgcccccccgccatctgaaagggttctaggggatttgcaacctctctcgtgtgtttcttctttccgagaagcgccgccacacgagaaagctggccgcgaaagtcgtgctggaatcacttccaacgaaaccccaggcatagatgggaaagggtgaagaacacgttgccatggctaccgtttccccggtcacggaataaacgctctctaggatccggaagtagttccgccgcgacctctctaaaaggatggatgtgttctctgcttacattcattggacgttttcccttagaggccaaggccgcccaggcaaaggggcggtcccacgcgtgaggggcccgcggagccatttgattggagaaaagctgcaaaccctgaccaatcggaaggagccacgcttcgggcatcggtcaccgcacctggacagctccgattggtggacttccgccccccctcacgaatcctcattgggtgccgtgggtgcgtggtgcggcgcgattggtgggttcatgtttcccgtcccccgcccgcgagaagtgggggtgaaaagcggcccgacctgcttggggtgtagtgggcggaccgcgcggctggaggtgtgaggatccgaacccaggggtggggggtggaggcggctcctgcgatcgaaggggacttgagactcaccggccgcacgtc

SEQ ID NO: 28 - DNA, 465 bp, synthetic construct
  source 1..465: mol_type = other DNA
gggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccc

SEQ ID NO: 29 - DNA, 538 bp, synthetic construct
  source 1..538: mol_type = other DNA
gccccacccccgtccgcgttacaaccgggaggcccgctgggtcctgcaccgtcaccctcctccctgtgaccgcccacctgatacccaaacaactttctcgcccctccagtccccagctcgccgagcgcttgcggggagccacccagcctcagtttccccagccccgggcggggcgaggggcgatgacgtcatgccggcgcgcggcattgtggggcggggcgaggcggggcgccggggggagcaacactgagacgccattttcggcggcgggagcggcgcaggcggccgagcgggactggctgggtcggctgggctgctggtgcgaggagccgcggggctgtgctcggcggccaaggggacagcgcgtgggtggccgaggatgctgcggggcggtagctccggcgcccctcgctggtgactgctgcgccgtgcctcacacagccgaggcgggctcggcgcacagtcgctgctccgcgctcgcgcccggcggcgctccaggtgctgacagcgcgagagagcgcggcctcaggagcaacac

SEQ ID NO: 30 - DNA, 1091 bp, synthetic construct
  source 1..1091: mol_type = other DNA
ttccagagctttcgaggaaggtttcttcaactcaaattcatccgcctgataattttcttatattttcctaaagaaggaagagaagcgcatagaggagaagggaaataattttttaggagcctttcttacggctatgaggaatttggggctcagttgaaaagcctaaactgcctctcgggaggttgggcgcggcgaactactttcagcggcgcacggagacggcgtctacgtgaggggtgataagtgacgcaacactcgttgcataaatttgcgctccgccagcccggagcatttaggggcggttggctttgttgggtgagcttgtttgtgtccctgtgggtggacgtggttggtgattggcaggatcctggtatccgctaacaggtactggcccacagccgtaaagacctgcgggggcgtgagaggggggaatgggtgaggtcaagctggaggcttcttggggttgggtgggccgctgaggggaggggagggcgaggtgacgcgacacccggcctttctgggagagtgggccttgttgacctaaggggggcgagggcagttggcacgcgcacgcgccgacagaaactaacagacattaaccaacagcgattccgtcgcgtttacttgggaggaaggcggaaaagaggtagtttgtgtggcttctggaaaccctaaatttggaatcccagtatgagaatggtgtcccttcttgtgtttcaatgggatttttacttcgcgagtcttgtgggtttggttttgttttcagtttgcctaacaccgtgcttaggtttgaggcagattggagttcggtcgggggagtttgaatatccggaacagttagtggggaaagctgtggacgcttggtaagagagcgctctggattttccgctgttgacgttgaaaccttgaatgacgaatttcgtattaagtgacttagccttgtaaaattgaggggaggcttgcggaatattaacgtatttaaggcattttgaaggaatagttgctaattttgaagaatattaggtgtaaaagcaagaaatacaatgatcctgaggtgacacgcttatgttttacttttaaactaggtcacc

SEQ ID NO: 31 - DNA, 66 bp, Homo sapiens
  source 1..66: mol_type = other DNA
atgtgtcaccagcagctcgttatatcctggtttagtttggtgtttctcgcttcacccctggtggca

SEQ ID NO: 32 - DNA, 66 bp, synthetic construct
  source 1..66: mol_type = other DNA
atgtgccatcagcaactcgtcatctcctggttctcccttgtgttcctcgcttcccctctggtcgcc

SEQ ID NO: 33 - DNA, 42 bp, synthetic construct
  source 1..42: mol_type = other DNA
atgcaactgctgtcatgtatcgcactcatcctggcgctggta

SEQ ID NO: 34 - DNA, 60 bp, Homo sapiens
  source 1..60: mol_type = other DNA
atgtatcggatgcaacttttgagctgcatcgcattgtctctggcgctggtgacaaattcc

SEQ ID NO: 35 - DNA, 45 bp, Homo sapiens
  source 1..45: mol_type = other DNA
atgaatctcttgctcatacttacgtttgtcgctgctgccgttgcg

SEQ ID NO: 36 - DNA, 51 bp, unidentified
  source 1..51: mol_type = other DNA; note = Gaussia Luciferase
atgggcgtgaaggtcttgtttgcccttatctgcatagctgttgcggaggcg

SEQ ID NO: 37 - DNA, 72 bp, Homo sapiens
  source 1..72: mol_type = other DNA
atgccgatggggagccttcaacctttggcaacgctttatcttctggggatgttggttgctagttgccttggg

SEQ ID NO: 38 - DNA, 63 bp, Mus musculus
  source 1..63: mol_type = other DNA
atggaaactgacacgttgttgctgtgggtattgctcttgtgggtcccaggatctacgggcgac

SEQ ID NO: 39 - DNA, 66 bp, Homo sapiens
  source 1..66: mol_type = other DNA
atggatatgagggttcccgcccagcttttggggctgcttttgttgtggcttcgaggggctcggtgt

SEQ ID NO: 40 - DNA, 48 bp, synthetic construct
  source 1..48: mol_type = other DNA
atgaagtgtctgttgtacctggcgtttctgttcattggtgtaaactgt

SEQ ID NO: 41 - DNA, 84 bp, Homo sapiens
  source 1..84: mol_type = other DNA
atgaatatcaaaggaagtccgtggaagggtagtctcctgctgctcctcgtatctaaccttctcctttgtcaatccgtggcaccc

SEQ ID NO: 42 - DNA, 54 bp, Homo sapiens
  source 1..54: mol_type = other DNA
atgaaatgggtaacattcatatcacttctctttctgttcagctctgcgtattct

SEQ ID NO: 43 - DNA, 57 bp, synthetic construct
  source 1..57: mol_type = other DNA
atgacaaggcttactgttttggctctcctcgctggactcttggcttcctcccgagca

SEQ ID NO: 44 - DNA, 51 bp, Homo sapiens
  source 1..51: mol_type = other DNA
atgagggcttggattttttttctgctctgccttgccggtcgagccctggcg

SEQ ID NO: 45 - DNA, 48 bp, Homo sapiens
  source 1..48: mol_type = other DNA
atgcctcttctgcttttgcttcctcttttgtgggcaggtgccctcgca

SEQ ID NO: 46 - DNA, 87 bp, Homo sapiens
  source 1..87: mol_type = other DNA
atgaactctttctcaacctctgcgtttggtccggtcgctttctcccttgggctcctgcttgtcttgccagcagcgtttcctgcgcca

SEQ ID NO: 47 - DNA, 60 bp, Homo sapiens
  source 1..60: mol_type = other DNA
atgacaagtaaactggcggtagccttgctcgcggcctttttgatttccgcagccctttgt

SEQ ID NO: 48 - DNA, 69 bp, Homo sapiens
  source 1..69: mol_type = other DNA
atgaaggtaagtgcagcgttgctttgccttctcctcattgcagcgacctttattcctcaagggctggcc

SEQ ID NO: 49 - DNA, 78 bp, Homo sapiens
  source 1..78: mol_type = other DNA
atgggagcggcagctagaacacttcgacttgcccttgggctcttgctccttgcaaccctccttagacctgccgacgca

SEQ ID NO: 50 - DNA, 63 bp, Homo sapiens
  source 1..63: mol_type = other DNA
atgtcaccgttgttgcggagattgctgttggccgcacttttgcaactggctcctgctcaagcc

SEQ ID NO: 51 - DNA, 63 bp, Homo sapiens
  source 1..63: mol_type = other DNA
atgaataacctgctctgttgtgcgctcgtgttcctggacatttctataaaatggacaacgcaa

SEQ ID NO: 52 - DNA, 69 bp, Homo sapiens
  source 1..69: mol_type = other DNA
atgcaaatgtctcctgcccttacctgtctcgtacttggtcttgcgctcgtatttggagagggatcagcc

SEQ ID NO: 53 - DNA, 102 bp, Homo sapiens
  source 1..102: mol_type = other DNA
atggcaagggctgcactcagtgctgccccgtctaatcccagattgcttcgagttgcattgcttcttctgttgctggttgcagctggtaggagagcagcgggt

SEQ ID NO: 54 - DNA, 63 bp, Homo sapiens
  source 1..63: mol_type = other DNA
atgaatgcaaaagtcgtggtcgtgctggttttggttctgacggcgttgtgtcttagtgatggg

SEQ ID NO: 55 - DNA, 66 bp, Homo sapiens
  source 1..66: mol_type = other DNA
atggaacgcattgtgatctgcctgatggtcatcttcctgggcaccttagtgcacaagtcgagcagc

SEQ ID NO: 56 - DNA, 1626 bp, Homo sapiens
  source 1..1626: mol_type = other DNA
atgtgccatcagcagcttgtcatatcttggttttcacttgtattcctggccagccctttggttgcgatctgggagctcaagaaggatgtgtacgttgtagagctggactggtaccccgatgctcccggtgagatggtcgttttgacatgtgacactccagaagaggacggtattacgtggactctggaccagtcctccgaagttcttggttctggtaagactctgactatccaggtgaaagaatttggggatgcgggacaatacacatgccacaagggaggcgaggtgttgtctcatagtttgctgcttctccacaagaaagaggatggaatctggagcaccgacatactcaaggatcaaaaggaacccaaaaataagacatttctgcgatgtgaggctaagaactatagtggccgcttcacttgttggtggctgactaccatcagcacagatctcacgttttcagtaaaaagtagtagaggttcaagtgatcctcaaggggtaacgtgcggtgctgcaacactgtctgctgaacgcgtaagaggagataataaggagtacgagtattccgtagaatgccaagaggacagtgcttgtcctgcggccgaggagtctctcccaatagaagtgatggtggacgcggtgcataaactgaaatatgagaactacacaagcagtttttttataagagatatcatcaagcccgatccgccgaagaatttgcaacttaaaccgcttaaaaactcacgccaggttgaagtatcctgggagtatccggatacatggtcaacaccacacagctatttttcccttaccttctgtgtgcaggtccaagggaagagcaaaagggagaagaaggacagggtattcactgataaaacttccgcgacggtcatctgccgaaaaaacgctagtatatctgtacgggcgcaggataggtactatagttcttcttggtctgagtgggcctcagttccgtgctctgggggaggaagtggaggagggtccggcggtggaagcgggggagggagtcgcaacttgccagtggctacaccagatccaggcatgtttccatgtctgcatcattcccagaatctcctgagagcggtgtcaaatatgctccaaaaagcgagacaaacactggaattttacccgtgtaccagtgaggagattgatcacgaggacataaccaaggacaagacctcaactgtagaagcgtgtttgccgctggagttgactaagaatgagtcctgcctcaattccagagaaacttcattcattactaacggcagttgtcttgcatcccggaaaacgtcctttatgatggccctttgccttagttcaatttacgaggatcttaaaatgtatcaagtggagtttaaaaccatgaatgctaaacttcttatggaccccaaacgacaaatttttctggatcagaatatgcttgccgtgatagacgaactcatgcaggcgcttaattttaactccgaaacagttccacaaaaatctagccttgaagaacctgatttttataaaacgaagattaaactgtgtatcctgctgcatgcctttcgcatccgagctgtcacaatcgatagggttatgtcctaccttaacgcgagctag

SEQ ID NO: 57 - DNA, 1623 bp, synthetic construct
  source 1..1623: mol_type = other DNA
atgtgccatcagcaactcgtcatctcctggttctcccttgtgttcctcgcttcccctctggtcgccatttgggaactgaagaaggacgtctacgtggtcgagctggattggtacccggacgcccctggagaaatggtcgtgctgacttgcgatacgccagaagaggacggcataacctggaccctggatcagagctccgaggtgctcggaagcggaaagaccctgaccattcaagtcaaggagttcggcgacgcgggccagtacacttgccacaagggtggcgaagtgctgtcccactccctgctgctgctgcacaagaaagaggatggaatctggtccactgacatcctcaaggaccaaaaagaaccgaagaacaagaccttcctccgctgcgaagccaagaactacagcggtcggttcacctgttggtggctgacgacaatctccaccgacctgactttctccgtgaagtcgtcacggggatcaagcgatcctcagggcgtgacctgtggagccgccactctgtccgccgagagagtcaggggagacaacaaggaatatgagtactccgtggaatgccaggaggacagcgcctgccctgccgcggaagagtccctgcctatcgaggtcatggtcgatgccgtgcataagctgaaatacgagaactacacttcctccttctttatccgcgacatcatcaagcctgacccccccaagaacttgcagctgaagccactcaagaactcccgccaagtggaagtgtcttgggaatatccagacacttggagcaccccgcactcatacttctcgctcactttctgtgtgcaagtgcagggaaagtccaaacgggagaagaaagaccgggtgttcaccgacaaaacctccgccactgtgatttgtcggaagaacgcgtcaatcagcgtccgggcgcaggatagatactactcgtcctcctggagcgaatgggccagcgtgccttgttccggtggcggatcaggcggaggttcaggaggaggctccggaggaggttcccggaacctccctgtggcaacccccgaccctggaatgttcccgtgcctacaccactcccaaaacctcctgagggctgtgtcgaacatgttgcagaaggcccgccagacccttgagttctacccctgcacctcggaagaaattgatcacgaggacatcaccaaggacaagacctcgaccgtggaagcctgcctgccgctggaactgaccaagaacgaatcgtgtctgaactcccgcgagacaagctttatcactaacggcagctgcctggcgtcgagaaagacctcattcatgatggcgctctgtctttcctcgatctacgaagatctgaagatgtatcaggtcgagttcaagaccatgaacgccaagctgctcatggacccgaagcggcagatcttcctggaccagaatatgctcgccgtgattgatgaactgatgcaggccctgaatttcaactccgagactgtgcctcaaaagtccagcctggaagaaccggacttctacaagaccaagatcaagctgtgcatcctgttgcacgctttccgcattcgagccgtgaccattgaccgcgtgatgtcctacctgaacgccagt

SEQ ID NO: 58 - DNA, 1635 bp, Mus musculus
  source 1..1635: mol_type = other DNA
atgtgtccacagaagctgacaataagttggtttgccattgtcctcctggtgagcccactcatggcaatgtgggaactcgaaaaggatgtctacgtggtagaagtagattggactccagacgcgccaggggagacagtgaatttgacatgtgacacaccagaagaagatgacattacatggacatctgaccaacgccatggcgtaatagggagtgggaaaacactcacgatcacagttaaagagttcttggatgctggtcaatatacttgccataaaggcggcgagacactcagccactcacatttgcttttgcataaaaaagagaatggcatttggagcactgaaatacttaagaactttaagaacaagacatttctcaagtgtgaggcccctaattacagcggcaggttcacgtgctcatggctggtccagcgcaacatggacctcaagtttaacataaaatcttcttcctcttcacctgactccagagctgttacttgcggcatggcttctctgagcgcagaaaaagtaacgttggatcaaagagactacgaaaagtactctgtttcttgtcaagaggatgttacgtgcccgacggccgaagaaacgcttccaattgaactcgcgttggaagctcgccaacaaaacaagtatgaaaactacagtacaagcttctttatacgggatataattaaacccgatccccccaagaacttgcaaatgaaaccacttaagaacagccaggtggaagtttcctgggagtatccagactcatggagtactcctcacagctatttttctctgaaattctttgtaaggatacaacggaagaaagagaagatgaaagagaccgaggagggttgtaatcagaagggagcgtttctcgtggagaaaacgtctaccgaagtccaatgtaaaggtggcaatgtgtgcgtccaagctcaggatagatactataattcaagttgctccaagtgggcctgtgttccatgccgcgttcggagcgggggaggtagcggaggaggtagtgggggtgggtcaggaggagggagtcgagttatcccggtgtcaggccccgcacgctgcttgagccagagtcgcaacctccttaagacaacagatgacatggtgaaaacagcacgcgaaaagcttaaacactactcttgtacggcggaggatattgatcacgaggatattacccgagaccaaactagcactttgaaaacctgtctgccccttgaacttcataaaaatgagagctgtctggctacacgagagacgtcaagtacgactaggggcagctgtctcccgccgcaaaagacaagcctcatgatgacgctctgtttgggttccatttacgaggacttgaaaatgtatcaaacggagttccaggctataaatgcggcgttgcagaaccataaccatcaacaaattatacttgataaaggcatgttggtggcgattgatgaactcatgcagagtctcaatcacaacggggaaacgttgagacagaaacccccagtcggtgaagcggacccatatcgagtaaaaatgaagctctgcattctgcttcacgcattcagcactagagttgttaccatcaaccgggtaatgggatatctctccagtgcgtag

SEQ ID NO: 59 - DNA, 465 bp, synthetic construct
  source 1..465: mol_type = other DNA
atggaacgcattgtgatctgcctgatggtcatcttcctgggcaccttagtgcacaagtcgagcagccagggacaggacaggcacatgattagaatgcgccagctcatcgatatcgtggaccagttgaagaactacgtgaacgacctggtgcccgagttcctgccggcccccgaagatgtggaaaccaattgcgaatggtcggcattttcctgctttcaaaaggcacagctcaagtccgctaacaccgggaacaacgaacggatcatcaacgtgtccatcaaaaagctgaagcggaagcctccctccaccaacgccggacggaggcagaagcataggctgacttgcccgtcatgcgactcctacgagaagaagccgccgaaggagttcctggagcggttcaagtcgctcctgcaaaagatgattcatcagcacctgtcctcccggactcatgggtctgaggattca

SEQ ID NO: 60 - DNA, 2163 bp, synthetic construct
  source 1..2163: mol_type = other DNA
atgtgccatcagcaactcgtcatctcctggttctcccttgtgttcctcgcttcccctctggtcgccatttgggaactgaagaaggacgtctacgtggtcgagctggattggtacccggacgcccctggagaaatggtcgtgctgacttgcgatacgccagaagaggacggcataacctggaccctggatcagagctccgaggtgctcggaagcggaaagaccctgaccattcaagtcaaggagttcggcgacgcgggccagtacacttgccacaagggtggcgaagtgctgtcccactccctgctgctgctgcacaagaaagaggatggaatctggtccactgacatcctcaaggaccaaaaagaaccgaagaacaagaccttcctccgctgcgaagccaagaactacagcggtcggttcacctgttggtggctgacgacaatctccaccgacctgactttctccgtgaagtcgtcacggggatcaagcgatcctcagggcgtgacctgtggagccgccactctgtccgccgagagagtcaggggagacaacaaggaatatgagtactccgtggaatgccaggaggacagcgcctgccctgccgcggaagagtccctgcctatcgaggtcatggtcgatgccgtgcataagctgaaatacgagaactacacttcctccttctttatccgcgacatcatcaagcctgacccccccaagaacttgcagctgaagccactcaagaactcccgccaagtggaagtgtcttgggaatatccagacacttggagcaccccgcactcatacttctcgctcactttctgtgtgcaagtgcagggaaagtccaaacgggagaagaaagaccgggtgttcaccgacaaaacctccgccactgtgatttgtcggaagaacgcgtcaatcagcgtccgggcgcaggatagatactactcgtcctcctggagcgaatgggccagcgtgccttgttccggtggcggatcaggcggaggttcaggaggaggctccggaggaggttcccggaacctccctgtggcaacccccgaccctggaatgttcccgtgcctacaccactcccaaaacctcctgagggctgtgtcgaacatgttgcagaaggcccgccagacccttgagttctacccctgcacctcggaagaaattgatcacgaggacatcaccaaggacaagacctcgaccgtggaagcctgcctgccgctggaactgaccaagaacgaatcgtgtctgaactcccgcgagacaagctttatcactaacggcagctgcctggcgtcgagaaagacctcattcatgatggcgctctgtctttcctcgatctacgaagatctgaagatgtatcaggtcgagttcaagaccatgaacgccaagctgctcatggacccgaagcggcagatcttcctggaccagaatatgctcgccgtgattgatgaactgatgcaggccctgaatttcaactccgagactgtgcctcaaaagtccagcctggaagaaccggacttctacaagaccaagatcaagctgtgcatcctgttgcacgctttccgcattcgagccgtgaccattgaccgcgtgatgtcctacctgaacgccagtagacggaaacgcggaagcggagagggcagaggctcgctgcttacatgcggggacgtggaagagaaccccggtccgatggaacgcattgtgatctgcctgatggtcatcttcctgggcaccttagtgcacaagtcgagcagccagggacaggacaggcacatgattagaatgcgccagctcatcgatatcgtggaccagttgaagaactacgtgaacgacctggtgcccgagttcctgccggcccccgaagatgtggaaaccaattgcgaatggtcggcattttcctgctttcaaaaggcacagctcaagtccgctaacaccgggaacaacgaacggatcatcaacgtgtccatcaaaaagctgaagcggaagcctccctccaccaacgccggacggaggcagaagcataggctgacttgcccgtcatgcgactcctacgagaagaagccgccgaaggagttcctggagcggttcaagtcgctcctgcaaaagatgattcatcagcacctgtcctcccggactcatgggtctgaggattca

SEQ ID NO: 61 - DNA, 2103 bp, Homo sapiens
  source 1..2103: mol_type = other DNA
atgtgccatcagcagcttgtcatatcttggttttcacttgtattcctggccagccctttggttgcgatctgggagctcaagaaggatgtgtacgttgtagagctggactggtaccccgatgctcccggtgagatggtcgttttgacatgtgacactccagaagaggacggtattacgtggactctggaccagtcctccgaagttcttggttctggtaagactctgactatccaggtgaaagaatttggggatgcgggacaatacacatgccacaagggaggcgaggtgttgtctcatagtttgctgcttctccacaagaaagaggatggaatctggagcaccgacatactcaaggatcaaaaggaacccaaaaataagacatttctgcgatgtgaggctaagaactatagtggccgcttcacttgttggtggctgactaccatcagcacagatctcacgttttcagtaaaaagtagtagaggttcaagtgatcctcaaggggtaacgtgcggtgctgcaacactgtctgctgaacgcgtaagaggagataataaggagtacgagtattccgtagaatgccaagaggacagtgcttgtcctgcggccgaggagtctctcccaatagaagtgatggtggacgcggtgcataaactgaaatatgagaactacacaagcagtttttttataagagatatcatcaagcccgatccgccgaagaatttgcaacttaaaccgcttaaaaactcacgccaggttgaagtatcctgggagtatccggatacatggtcaacaccacacagctatttttcccttaccttctgtgtgcaggtccaagggaagagcaaaagggagaagaaggacagggtattcactgataaaacttccgcgacggtcatctgccgaaaaaacgctagtatatctgtacgggcgcaggataggtactatagttcttcttggtctgagtgggcctcagttccgtgctctgggggaggaagtggaggagggtccggcggtggaagcgggggagggagtcgcaacttgccagtggctacaccagatccaggcatgtttccatgtctgcatcattcccagaatctcctgagagcggtgtcaaatatgctccaaaaagcgagacaaacactggaattttacccgtgtaccagtgaggagattgatcacgaggacataaccaaggacaagacctcaactgtagaagcgtgtttgccgctggagttgactaagaatgagtcctgcctcaattccagagaaacttcattcattactaacggcagttgtcttgcatcccggaaaacgtcctttatgatggccctttgccttagttcaatttacgaggatcttaaaatgtatcaagtggagtttaaaaccatgaatgctaaacttcttatggaccccaaacgacaaatttttctggatcagaatatgcttgccgtgatagacgaactcatgcaggcgcttaattttaactccgaaacagttccacaaaaatctagccttgaagaacctgatttttataaaacgaagattaaactgtgtatcctgctgcatgcctttcgcatccgagctgtcacaatcgatagggttatgtcctaccttaacgcgagccggcgcaagaggggttccggagagggaaggggtagtctgctcacctgcggcgatgttgaagaaaatcctggtcccatggcgcaaagtctggctctttcactcctgatcctggtcttggccttcgggattccgaggacccaaggaagtgatggtggcgcccaagattgttgccttaaatacagccagcggaaaatacccgcgaaagtggtcaggagttatagaaaacaggagccttccctgggttgtagtatccccgccatacttttcctcccgagaaaacggagccaggccgaactgtgcgctgaccctaaggaactttgggtgcaacaacttatgcaacacctggataagacaccttctcctcaaaagccagctcagggctgccgaaaagatagaggcgcctcaaaaaccggaaaaaagggcaaaggttctaaaggatgtaagcggactgaacgctctcaaacgcctaaagggccgtag

SEQ ID NO: 62 - DNA, 2109 bp, Mus musculus
  source 1..2109: mol_type = other DNA
atgtgtccacagaagctgacaataagttggtttgccattgtcctcctggtgagcccactcatggcaatgtgggaactcgaaaaggatgtctacgtggtagaagtagattggactccagacgcgccaggggagacagtgaatttgacatgtgacacaccagaagaagatgacattacatggacatctgaccaacgccatggcgtaatagggagtgggaaaacactcacgatcacagttaaagagttcttggatgctggtcaatatacttgccataaaggcggcgagacactcagccactcacatttgcttttgcataaaaaagagaatggcatttggagcactgaaatacttaagaactttaagaacaagacatttctcaagtgtgaggcccctaattacagcggcaggttcacgtgctcatggctggtccagcgcaacatggacctcaagtttaacataaaatcttcttcctcttcacctgactccagagctgttacttgcggcatggcttctctgagcgcagaaaaagtaacgttggatcaaagagactacgaaaagtactctgtttcttgtcaagaggatgttacgtgcccgacggccgaagaaacgcttccaattgaactcgcgttggaagctcgccaacaaaacaagtatgaaaactacagtacaagcttctttatacgggatataattaaacccgatccccccaagaacttgcaaatgaaaccacttaagaacagccaggtggaagtttcctgggagtatccagactcatggagtactcctcacagctatttttctctgaaattctttgtaaggatacaacggaagaaagagaagatgaaagagaccgaggagggttgtaatcagaagggagcgtttctcgtggagaaaacgtctaccgaagtccaatgtaaaggtggcaatgtgtgcgtccaagctcaggatagatactataattcaagttgctccaagtgggcctgtgttccatgccgcgttcggagcgggggaggtagcggaggaggtagtgggggtgggtcaggaggagggagtcgagttatcccggtgtcaggccccgcacgctgcttgagccagagtcgcaacctccttaagacaacagatgacatggtgaaaacagcacgcgaaaagcttaaacactactcttgtacggcggaggatattgatcacgaggatattacccgagaccaaactagcactttgaaaacctgtctgccccttgaacttcataaaaatgagagctgtctggctacacgagagacgtcaagtacgactaggggcagctgtctcccgccgcaaaagacaagcctcatgatgacgctctgtttgggttccatttacgaggacttgaaaatgtatcaaacggagttccaggctataaatgcggcgttgcagaaccataaccatcaacaaattatacttgataaaggcatgttggtggcgattgatgaactcatgcagagtctcaatcacaacggggaaacgttgagacagaaacccccagtcggtgaagcggacccatatcgagtaaaaatgaagctctgcattctgcttcacgcattcagcactagagttgttaccatcaaccgggtaatgggatatctctccagtgcgcggcgcaagaggggttccggagagggaaggggtagtctgctcacctgcggcgatgttgaagaaaatcctggtcccatggcgcaaatgatgaccctttccctgctgagtcttgtcctcgcgctctgcatcccgtggacgcaggggtctgatggggggggccaagactgttgcctgaagtattcacaaaaaaagataccgtactctattgtcagagggtacaggaagcaagaaccctccttgggttgccctataccagcaattcttttctccccacgcaagcattccaaaccagaactgtgtgcgaaccccgaggagggttgggtacagaacttgatgcgaaggcttgaccagcccccagcccctggcaagcagtcacctgggtgcagaaaaaacagaggtacttcaaagagcggcaagaaaggcaaagggagtaaaggatgtaaaagaacggagcagacccagccttcacgaggctag

SEQ ID NO: 63 - DNA, 2109 bp, Mus musculus
  source 1..2109: mol_type = other DNA
atggcgcaaatgatgaccctttccctgctgagtcttgtcctcgcgctctgcatcccgtggacgcaggggtctgatggggggggccaagactgttgcctgaagtattcacaaaaaaagataccgtactctattgtcagagggtacaggaagcaagaaccctccttgggttgccctataccagcaattcttttctccccacgcaagcattccaaaccagaactgtgtgcgaaccccgaggagggttgggtacagaacttgatgcgaaggcttgaccagcccccagcccctggcaagcagtcacctgggtgcagaaaaaacagaggtacttcaaagagcggcaagaaaggcaaagggagtaaaggatgtaaaagaacggagcagacccagccttcacgaggccggcgcaagaggggttccggagagggaaggggtagtctgctcacctgcggcgatgttgaagaaaatcctggtcccatgtgtccacagaagctgacaataagttggtttgccattgtcctcctggtgagcccactcatggcaatgtgggaactcgaaaaggatgtctacgtggtagaagtagattggactccagacgcgccaggggagacagtgaatttgacatgtgacacaccagaagaagatgacattacatggacatctgaccaacgccatggcgtaatagggagtgggaaaacactcacgatcacagttaaagagttcttggatgctggtcaatatacttgccataaaggcggcgagacactcagccactcacatttgcttttgcataaaaaagagaatggcatttggagcactgaaatacttaagaactttaagaacaagacatttctcaagtgtgaggcccctaattacagcggcaggttcacgtgctcatggctggtccagcgcaacatggacctcaagtttaacataaaatcttcttcctcttcacctgactccagagctgttacttgcggcatggcttctctgagcgcagaaaaagtaacgttggatcaaagagactacgaaaagtactctgtttcttgtcaagaggatgttacgtgcccgacggccgaagaaacgcttccaattgaactcgcgttggaagctcgccaacaaaacaagtatgaaaactacagtacaagcttctttatacgggatataattaaacccgatccccccaagaacttgcaaatgaaaccacttaagaacagccaggtggaagtttcctgggagtatccagactcatggagtactcctcacagctatttttctctgaaattctttgtaaggatacaacggaagaaagagaagatgaaagagaccgaggagggttgtaatcagaagggagcgtttctcgtggagaaaacgtctaccgaagtccaatgtaaaggtggcaatgtgtgcgtccaagctcaggatagatactataattcaagttgctccaagtgggcctgtgttccatgccgcgttcggagcgggggaggtagcggaggaggtagtgggggtgggtcaggaggagggagtcgagttatcccggtgtcaggccccgcacgctgcttgagccagagtcgcaacctccttaagacaacagatgacatggtgaaaacagcacgcgaaaagcttaaacactactcttgtacggcggaggatattgatcacgaggatattacccgagaccaaactagcactttgaaaacctgtctgccccttgaacttcataaaaatgagagctgtctggctacacgagagacgtcaagtacgactaggggcagctgtctcccgccgcaaaagacaagcctcatgatgacgctctgtttgggttccatttacgaggacttgaaaatgtatcaaacggagttccaggctataaatgcggcgttgcagaaccataaccatcaacaaattatacttgataaaggcatgttggtggcgattgatgaactcatgcagagtctcaatcacaacggggaaacgttgagacagaaacccccagtcggtgaagcggacccatatcgagtaaaaatgaagctctgcattctgcttcacgcattcagcactagagttgttaccatcaaccgggtaatgggatatctctccagtgcgtag

SEQ ID NO: 64 - DNA, 465 bp, Mus musculus
  source 1..465: mol_type = other DNA
atgtttcatgtgtccttcaggtacatatttggtatcccaccacttatattggtgctcttgcctgtaaccagctctgaatgtcatataaaagacaaggagggcaaagcatacgagtccgtattgatgatctcaatcgatgaacttgacaagatgacagggaccgattctaattgtccaaataacgagccaaacttctttcggaaacacgtgtgtgatgatacaaaagaagctgcttttcttaacagagctgccagaaaactcaagcagttcctcaagatgaatatatccgaggaatttaacgtgcatctcctcacagtatctcagggaactcaaacccttgtaaactgcacttctaaggaggagaagaatgtcaaagagcagaagaaaaatgatgcatgttttttgaaacggctgttgagggagatcaaaacatgctggaataaaatcctcaagggctcaatttag

SEQ ID NO: 65 - DNA, 1428 bp, Homo sapiens
  source 1..1428: mol_type = other DNA
atggaaacagacacattgctgctttgggtattgttgctctgggtgcctggatcaacaggaaactgggtaaacgtaatttcagatctgaagaagatcgaggaccttattcaatccatgcacatcgatgccactctctacaccgaaagcgacgttcacccatcttgcaaggtgaccgctatgaaatgtgaattgttggaacttcaggtaatttctctggagagcggcgatgcctcaatacatgacaccgttgaaaatcttatcatccttgctaatgattcactctctagtaatgggaacgtaacagagagcgggtgtaaggagtgtgaagaactggaggagaaaaacattaaggaatttttgcagtcattcgtccatatagtgcaaatgttcataaacacttccagaagaaagcgaggctctggggaggggcgaggctctctgctgacctgtggggatgtagaagagaatccaggtcccatggaccggctgaccagctcattcctgcttctgattgtgccagcctacgtgctctccatcacatgtcctcccccaatgagcgtcgagcatgctgacatctgggtgaagtcatactccttgtacagcagagagagatacatttgtaattccggattcaagcgcaaggccggcacctcctctctgacagagtgcgtccttaacaaagcaaccaacgtagcacattggaccacaccatccttgaagtgcatacgagaacctaaatcttgcgataagactcatacttgtccaccttgtccagccccagaactgcttggcggaccctcagtatttttgttcccaccaaagccaaaagacacactcatgatatccagaactcctgaggtgacctgtgtcgttgtagacgtttcccacgaagatcctgaagtaaaattcaactggtacgtggatggggtcgaagtccataacgccaagactaaaccaagggaggaacagtataactctacttaccgagtagtttctgtgttgaccgtgctgcaccaggactggttgaacgggaaggagtacaaatgcaaggtgagcaataaagctctgcccgcaccaatcgaaaagacaatatctaaggccaaggggcagccacgagagccccaggtatacacactgccaccctcacgcgatgaattgactaagaaccaggtttccctgacctgtcttgtaaaaggtttctacccttccgacatagctgttgagtgggaaagtaacgggcagccagagaacaattacaagacaactccacccgttcttgatagcgatggatcattttttctgtattccaaactcactgtcgataaaagtcgctggcagcaaggcaatgtttttagctgctcagtcatgcacgaagcactgcataatcactacacacaaaaaagtttgtcccttagccctggtaagtag

SEQ ID NO: 66 - DNA, 1080 bp, Homo sapiens
  source 1..1080: mol_type = other DNA
atgtactcaatgcagttggcctcctgtgtaacattgaccttggtcctcttggtcaacagcaattggatcgatgtacgctacgacttggagaagattgagtcccttatacagagtatacacatagatacaaccttgtatactgacagtgacttccatcccagctgtaaagtgactgcaatgaactgttttttgttggagttgcaagtaattctgcatgaatacagcaacatgaccctcaatgaaaccgttaggaatgtcctttatctcgcaaattctactctgagtagcaataagaatgttgccgaaagcggctgcaaggagtgcgaagaactggaggaaaaaactttcaccgagtttctccagagtttcatcagaattgtccaaatgttcattaatacaagtagtggtggtgggagcgggggtggaggcagtgggggaggtgggagcggaggtggagggtccggaggggggagccttcaaggcactacttgtcctccacccgtatccatcgagcacgccgatattcgagttaaaaattatagtgttaatagcagagaacgatacgtctgcaactcagggtttaagagaaaggccggaacttcaactctcatagaatgcgtgattaataagaatactaacgtcgcacattggactactcccagtctcaagtgcatacgcgatccatctctcgctcattactcaccagtacctacagtggttactcctaaggtgacctctcagcccgaatcaccatctcccagcgcaaaagagcctgaggccttttctcctaaatcagacactgctatgactacagaaacagccataatgccaggaagccggctgacaccatctcaaactaccagcgcaggcacaactgggactggctcccacaaaagctcacgcgcaccaagtctcgccgcaacaatgacattggagcctacagccagcacatctcttagaatcacagaaatttctccccacagtagcaagatgaccaaggtggcaattagtaccagcgtccttcttgtaggagctggagttgtgatggcatttttggcatggtatatcaaaagcaggtag

SEQ ID NO: 67 - DNA, 489 bp, Mus musculus
  source 1..489: mol_type = other DNA
atgaagatcctcaagccatacatgcgaaacactagtattagctgttacttgtgttttctgctgaatagtcattttttgactgaagcaggaatccatgtatttatactcggttgtgtgtctgtaggtctgccaaagactgaggctaattggattgacgtgcgctatgatcttgaaaaaatagagtccttgattcaatcaatacacatcgataccactctctacaccgacagtgatttccatccttcctgcaaggtaacagctatgaattgcttcctcctggagctccaagtcattctccatgagtactccaacatgactttgaacgaaactgtaagaaacgtattgtatctggctaatagcaccttgtctagtaacaaaaatgtggcagagagcggctgcaaagaatgtgaagaattggaagagaaaacatttacagagttcctgcaatcctttattcgcatcgtccaaatgtttatcaatacctcttag

SEQ ID NO: 68 - DNA, 438 bp, Mus musculus
  source 1..438: mol_type = other DNA
atgtattccatgcaacttgccagttgtgtaacccttactctcgtcctgctcgttaattccgctggtgctaactggatagatgttcgatacgatctggaaaagattgagtcccttatccaatccattcatatagataccaccctttatactgacagcgacttccatccttcttgcaaggtgaccgctatgaattgtttcctgctggaactccaagttattctgcatgaatactctaatatgacacttaacgagaccgtaagaaatgttctctatctcgctaatagtactttgagctcaaataagaacgtggccgagtctgggtgtaaggaatgcgaagagctggaagaaaagacattcaccgagtttctccagtctttcatacggattgtgcagatgtttatcaacacatcagattacaaagacgacgatgataagtag

SEQ ID NO: 69 - DNA, 579 bp, Mus musculus
  source 1..579: mol_type = other DNA
atggcagccatgtctgaggactcttgtgtgaactttaaagaaatgatgttcatagacaatacactctactttatacctgaggagaatggagatttggaatctgacaactttggcaggctgcattgcactaccgcagttatccgaaacatcaacgatcaggtactgtttgttgataaaagacaacctgtattcgaggacatgaccgacatagatcagtctgcctcagagccccagactaggcttatcatctatatgtacaaggacagcgaagtacgaggcctggctgttacactctcagtcaaagactctaagatgagcaccctgtcatgcaagaacaaaattatcagttttgaggagatggacccacctgaaaacatagatgacattcagtcagacctcattttttttcaaaagcgggtaccaggacacaacaaaatggaatttgaatcatcactctacgaaggacatttccttgcatgccagaaagaggatgacgcattcaaattgatcctgaaaaaaaaggacgaaaatggtgataaatcagtcatgtttacattgaccaatcttcaccaaagttag

SEQ ID NO: 70 - DNA, 579 bp, Mus musculus
  source 1..579: mol_type = other DNA
atggctgcaatgtctgaagatagctgtgtcaactttaaggagatgatgttcattgataatactttgtactttatacctgaagaaaatggagaccttgagtcagacaacttcgggagactgcactgcacaactgccgttatccgaaacataaatgatcaagtattgttcgtggacaaaagacaaccagtctttgaggatatgacagacatcgaccaatccgcatctgaacctcagactaggctgatcatctatatgtacgccgactccgaagtaagaggccttgctgtgacacttagtgttaaggatagtaagatgagcacactgtcctgtaagaataagattatatcttttgaagagatggaccctcccgagaacatagatgacatccagagcgacttgatcttctttcagaagcgagtgccaggccataacaagatggaatttgaatcatctctttatgaaggccatttcctcgcatgtcaaaaggaggacgatgccttcaagctcattctgaaaaaaaaagacgagaacggtgataagagcgtgatgttcactctgacaaatctgcaccagtcatag

SEQ ID NO: 71 - DNA, 534 bp, Homo sapiens
  source 1..534: mol_type = other DNA
atgtatcgcatgcaactcctgtcctgcattgctctgagcttggctttggtaaccaactcatacttcgggaaactggagagtaaactctccgtaatcaggaatcttaatgaccaagtattgtttattgaccagggcaaccgcccgttgttcgaggatatgactgattctgactgtcgggataacgctccgagaactatctttatcatttcaatgtacaaggacagccaaccgcggggtatggctgtgacaatcagtgtcaaatgtgagaagatttccacgctgtcctgcgaaaacaagataatttctttcaaagaaatgaacccccctgacaatataaaggatacaaagagtgatatcatcttctttcagaggtccgtgcccggccacgataataagatgcaatttgaaagttcatcttatgaggggtactttttggcatgcgagaaagaaagggatctcttcaagttgatcctgaagaaggaggacgaattgggcgaccgctccatcatgttcacagtccagaacgaggactag

SEQ ID NO: 72 - DNA, 534 bp, Homo sapiens
  source 1..534: mol_type = other DNA
atgtaccgcatgcagctcctgagttgtattgccctttccctcgctctcgttaccaattcttacttcggtaagcttgcctctaaactctctgttattaggaacttgaacgaccaagtccttttcatagaccaagggaacagaccactgtttgaagatatgacggatagcgattgccgagataatgcccctaggacgatttttatcattagtatgtatgcggactctcaaccgagggggatggccgttactataagtgtgaaatgcgagaaaatatcaacgctcagttgtgagaacaaaatcataagtttcaaggagatgaatccacctgataacatcaaagacactaagtctgatattatatttttccaacgaagtgttccgggacacgataacaaaatgcaatttgagagctcctcatacgagggctacttcctcgcgtgtgagaaagaaagggatttgtttaagcttatcctcaagaaagaggacgagttgggggatcggagcataatgtttaccgtacagaatgaggactag

SEQ ID NO: 73 - DNA, 441 bp, Mus musculus
  source 1..441: mol_type = other DNA
atggagcggacactcgtgtgtcttgtcgtaatttttctcgggacagtcgcacacaagtcctcaccccagggtcctgatcgccttctcatacgcctccgacatttgatcgacattgtagagcagctcaaaatttacgagaatgacctcgatcccgagcttttgagtgctccccaagacgttaagggtcattgcgagcacgcagcttttgcttgcttccagaaggccaagttgaaaccaagcaaccctggtaataataagactttcatcatcgacttggtcgcccaactccgaaggaggctgcctgcccggcgcggaggaaaaaaacaaaagcatattgcaaagtgtccttcatgtgattcatacgaaaagcggactcccaaagagttcttggaaaggttgaaatggcttcttcagaagatgattcatcaacatttgtcatag

SEQ ID NO: 74 - DNA, 564 bp, Homo sapiens
  source 1..564: mol_type = other DNA
atgaccaacaaatgccttttgcaaattgccctgcttttgtgttttagcactaccgcattgagcatgtcatataacctcctcggcttccttcagagatcatcaaactttcagtgtcagaaactgctttggcaacttaatggcaggctcgaatattgtctgaaagatcggatgaatttcgacattccagaagaaataaaacagcttcaacaattccagaaagaggacgccgccctgactatttacgagatgctccagaatatcttcgccattttccggcaggacagctcatccacggggtggaatgagactattgtagaaaatcttctggctaatgtgtaccatcaaattaatcacctcaaaacggtgcttgaggaaaaacttgaaaaggaagatttcacacggggcaagttgatgtcctccctgcaccttaaacgatactacggcaggattcttcattacttgaaggctaaggagtatagccattgcgcgtggacaattgtacgggtagaaatactgcgaaacttttatttcatcaaccggctcactggataccttagaaattag

SEQ ID NO: 75 - DNA, 549 bp, Mus musculus
  source 1..549: mol_type = other DNA
atgaacaatcggtggatactccacgccgcatttctcctctgctttagcacgacggccctgtccatcaactacaaacagcttcagttgcaggagcggactaacataaggaagtgccaggaactgctggaacagcttaatggtaaaattaatcttacataccgagctgacttcaaaattcctatggaaatgaccgagaagatgcagaaatcctacacggcattcgccatccaggaaatgctccagaacgtatttctcgtgttccgcaataatttctcttctacgggttggaacgaaaccattgttgttagactgcttgacgaactgcatcagcaaaccgtgttccttaaaaccgtgcttgaggagaagcaggaggagcgcctgacttgggagatgtctagtaccgcacttcacttgaaatcctactactggcgcgttcagcggtatctgaagctgatgaagtataactcatacgcctggatggtagtgcgcgcagagatcttcagaaactttcttatcatccggcgactgacccgaaactttcagaattag

SEQ ID NO: 76 - DNA, 501 bp, Homo sapiens
  source 1..501: mol_type = other DNA
atgaagtacactagctatatattggccttccagctttgcatcgtattgggtagcctcggatgctattgccaagacccgtatgtcaaagaagccgaaaatctcaaaaagtatttcaatgccggacactcagacgtcgcggataacggtacactgtttcttggcatcctgaaaaattggaaggaagagagtgacagaaaaataatgcagtcacaaatagtgtccttttactttaagctgttcaaaaatttcaaggatgaccaaagtatccagaagagtgttgaaactatcaaagaggacatgaatgtgaaattctttaacagtaataagaagaagcgcgatgacttcgagaaactcactaattacagcgtaacggatcttaacgtccaacgcaaggcaatccacgagcttatacaggtaatggctgagcttagtcccgcagccaagacagggaagagaaaaaggtctcaaatgctttttcggggccggcgagcttcacaatag

SEQ ID NO: 77 - DNA, 468 bp, Mus musculus
  source 1..468: mol_type = other DNA
atgaacgctacgcattgcatcctcgcactccaattgttcctcatggctgtgtcagggtgttactgtcacggtactgtcatagaaagcctcgaatccctgaataactattttaacagtagcggtatagatgtagaagaaaagtctctctttcttgacatctggaggaattggcaaaaggatggagacatgaagattctccaatctcagattatatcattttacttgaggctttttgaggttctgaaggataaccaggcgatcagcaataatatcagcgtaattgaatctcaccttattacaacatttttctcaaattccaaggcaaagaaagatgctttcatgtctatcgcgaaatttgaggtgaacaatcctcaggtacaaaggcaagcctttaacgagctgattagagttgtacatcagttgttgcccgaaagtagtcttagaaaacgcaaacggagccgatgctag

SEQ ID NO: 78 - DNA, 570 bp, Mus musculus
  source 1..570: mol_type = other DNA
atggcaaggttgtgcgcttttctcatggtactggctgtgctctcctattggcctacttgttctctgggatgcgacttgccacagacccacaatctgcggaataagagggctctgactctgctggtgcaaatgagacggctctctccacttagctgtttgaaagatagaaaggatttcgggttcccccaggagaaggtggatgcccagcagatcaagaaggcacaggctatccccgtcctttccgagctgacccagcaaattttgaacatctttacaagtaaggatagttcagctgcatggaataccacacttttggattctttttgtaacgatctgcatcagcagctgaacgatctccagggatgcctgatgcagcaagtcggcgtgcaagaatttccactcacccaggaggacgctctgctcgcagtgcgaaagtattttcaccgaattaccgtgtacctccgggagaaaaagcattcaccctgcgcttgggaagtagtcagggccgaagtatggagagcccttagtagctccgctaatgtactgggccggttgcgggaagagaaatag

SEQ ID NO: 79 - DNA, 405 bp, Homo sapiens
  source 1..405: mol_type = other DNA
atggcgcaaagtctggctctttcactcctgatcctggtcttggccttcgggattccgaggacccaaggaagtgatggtggcgcccaagattgttgccttaaatacagccagcggaaaatacccgcgaaagtggtcaggagttatagaaaacaggagccttccctgggttgtagtatccccgccatacttttcctcccgagaaaacggagccaggccgaactgtgcgctgaccctaaggaactttgggtgcaacaacttatgcaacacctggataagacaccttctcctcaaaagccagctcagggctgccgaaaagatagaggcgcctcaaaaaccggaaaaaagggcaaaggttctaaaggatgtaagcggactgaacgctctcaaacgcctaaagggccgtag

SEQ ID NO: 80 - DNA, 402 bp, Mus musculus
  source 1..402: mol_type = other DNA
atggcgcaaatgatgaccctttccctgctgagtcttgtcctcgcgctctgcatcccgtggacgcaggggtctgatggggggggccaagactgttgcctgaagtattcacaaaaaaagataccgtactctattgtcagagggtacaggaagcaagaaccctccttgggttgccctataccagcaattcttttctccccacgcaagcattccaaaccagaactgtgtgcgaaccccgaggagggttgggtacagaacttgatgcgaaggcttgaccagcccccagcccctggcaagcagtcacctgggtgcagaaaaaacagaggtacttcaaagagcggcaagaaaggcaaagggagtaaaggatgtaaaagaacggagcagacccagccttcacgaggctag

SEQ ID NO: 81 - DNA, 309 bp, Homo sapiens
  source 1..309: mol_type = other DNA
atggcgcaaagtctggctctttcactcctgatcctggtcttggccttcgggattccgaggacccaaggaagtgatggtggcgcccaagattgttgccttaaatacagccagcggaaaatacccgcgaaagtggtcaggagttatagaaaacaggagccttccctgggttgtagtatccccgccatacttttcctcccgagaaaacggagccaggccgaactgtgcgctgaccctaaggaactttgggtgcaacaacttatgcaacacctggataagacaccttctcctcaaaagccagctcagggctag

SEQ ID NO: 82 - DNA, 309 bp, Mus musculus
  source 1..309: mol_type = other DNA
atggcgcaaatgatgaccctttccctgctgagtcttgtcctcgcgctctgcatcccgtggacgcaggggtctgatggggggggccaagactgttgcctgaagtattcacaaaaaaagataccgtactctattgtcagagggtacaggaagcaagaaccctccttgggttgccctataccagcaattcttttctccccacgcaagcattccaaaccagaactgtgtgcgaaccccgaggagggttgggtacagaacttgatgcgaaggcttgaccagcccccagcccctggcaagcagtcacctgggtag

SEQ ID NO: 83 - DNA, 327 bp, Mus musculus
  source 1..327: mol_type = other DNA
atggcaccccgcgtcacacccttgcttgctttttctctgcttgtcctctggaccttccccgctcctacccttggaggagccaatgatgccgaggattgctgcctgagtgttacacaaaggccaataccagggaatatagtgaaggcattccggtatctgctcaatgaagatgggtgcagagtccccgcagttgtctttacaacattgcgaggttaccagctttgtgctcccccagaccagccttgggtagatcgcattattcgccggttgaagaagagctcagcaaagaataagggcaattccacacggagaagccccgtctcctag

SEQ ID NO: 84 - DNA, 381 bp, Mus musculus
  source 1..381: mol_type = other DNA
atgaaatcagcagtccttttcttgctcgggattatttttctggaacaatgtggagtgaggggaacactcgtaataagaaacgctcggtgctcatgcatatcaacatcacggggcactatccactacaaatccctgaaggatctgaagcagttcgccccaagccctaactgtaacaagaccgaaattatcgcaactctcaaaaatggagatcagacttgtcttgacccagattcagcaaatgtcaagaagctgatgaaagagtgggaaaagaagatttcacaaaaaaaaaagcaaaaacgcggcaagaaacatcaaaagaacatgaaaaacaggaaacctaagactccccagtcaaggagaagatcccgcaagacaacctag

SEQ ID NO: 85 - DNA, 303 bp, Mus musculus
  source 1..303: mol_type = other DNA
atgaacagaaaagttaccgctatagcacttgctgccataatatgggccaccgcagctcaagggttcctgatgttcaagcagggccgatgcctctgcattggccctggaatgaaggccgtgaaaatggccgaaatagaaaaagctagtgtcatatacccctctaacggttgcgataaagtcgaggttatagtcacaatgaaagctcataaacgccaacgctgcctcgacccccggtctaagcaggctaggctcataatgcaagcaatcgagaagaaaaactttcttagacggcaaaacatgtag

SEQ ID NO: 86 - DNA, 297 bp, Mus musculus
  source 1..297: mol_type = other DNA
atgaacccatctgccgccgttattttctgtctgatactccttgggctgagtggcacacaaggcatacccctcgcccgcacagtccggtgtaattgtatacatattgacgacggccctgttagaatgcgggccatcggtaagctggagattataccagcaagccttagttgtcccagggttgaaatcatagcaactatgaaaaaaaacgacgaacaaagatgtttgaatcccgagagcaagacaatcaaaaaccttatgaaagcatttagtcaaaaacgctctaaacgcgctccatag

SEQ ID NO: 87 - DNA, 297 bp, Homo sapiens
  source 1..297: mol_type = other DNA
atgaatcagacggcaatccttatatgctgccttatattccttactctctcagggatacaaggggtaccactttctcggactgttcgctgcacttgcatttcaatatctaaccaacctgtaaatccgcggagcctggaaaaattggagattatacctgcttctcaattctgccctcgggtggaaatcatcgccactatgaagaagaagggcgagaaaaggtgtctgaatccagagtcaaaggcaatcaaaaacctgctgaaagcggtgtcaaaggaacggtccaagagatcaccctag

SEQ ID NO: 88 - DNA, 534 bp, Mus musculus
  source 1..534: mol_type = other DNA
atgaacaggaaagtaacagccattgcattggctgccatcatctgggccaccgcagcacagggttttctgatgtttaagcaagggcgctgtctctgtataggcccaggcatgaaggccgtgaagatggcagagattgagaaggcatctgtgatttatccttctaacgggtgcgataaagtcgaagttattgtgacaatgaaggcacacaaacgccaacggtgtttggacccacgatctaaacaggcaagattgattatgcaagccatcgagaaaaagaactttctccgaaggcaaaatatgatccctttggctcggacagtgcggtgtaactgtattcacatcgacgatgggccagtacggatgagagcaataggaaagctcgaaatcatacccgcctcattgtcttgtcccagggtggaaataatcgccactatgaaaaagaacgatgaacagaggtgtctcaacccagagagtaagactatcaagaaccttatgaaggcattcagtcagaagaggtcaaagcgagcaccatag

SEQ ID NO: 89 - DNA, 345 bp, Homo sapiens
  source 1..345: mol_type = other DNA
atgagacttctcatattggcgcttctcgggatatgttctcttacggcatacatagttgagggggtgggatctgaggttagcgataaacgaacttgtgttagtcttacaacacagaggcttccagtctccaggataaaaacatatacgataactgagggatctctcagagcggtcatcttcataacgaagaggggcctgaaggtctgtgctgacccacaagcgacttgggtaagggacgttgtgcggagcatggacaggaagagcaatactcgcaacaacatgatccaaaccaaacctacgggcacccaacagtcaaccaatactgcggtaacattgacggggtag

SEQ ID NO: 90 - DNA, 345 bp, Mus musculus
  source 1..345: mol_type = other DNA
atgcgcctccttctgctgacttttctgggtgtatgttgcctgacaccctgggtcgtagaaggagtaggaaccgaggttctggaagagtcctcatgtgtaaacttgcagacacaacgactccccgtccaaaaaatcaagacctatataatctgggagggggcaatgcgggccgtcattttcgtgactaaacgaggtctcaaaatctgcgccgaccccgaggctaagtgggtgaaggcagccattaagaccgtggatgggagagccagcaccagaaagaacatggccgaaacagtacctactggcgcacagcggtcaacctcaactgctataaccttgacaggatag

SEQ ID NO: 91 - DNA, 513 bp, synthetic construct
  source 1..513: mol_type = other DNA
atggagactgacactctgcttctgtgggtgttgctgctgtgggtgcctggcagtacaggcgatatgcaacgaggtgacgaggaccctcaaatcgccgcccatgtagtctctgaagctaatagcaacgctgcatccgtcttgcagtgggcaaagaaaggctactatactatgaagtccaacttggtaatgcttgaaaacggcaagcagttgactgtcaagagagagggactttattacgtctatacccaagtcacattctgtagcaatcgagaaccctcctcacagaggccttttatagtgggactctggcttaaaccaagtagcggctctgagcgcatactgttgaaagccgcaaacacacacagctcttcccaactctgcgagcagcaatccgtgcatctcggtggagtatttgagcttcaagccggtgcctcagtgtttgtgaacgtcactgaggcctcccaggtcatacatcgagttgggttcagctccttcggcttgctcaagctctag

SEQ ID NO: 92 - DNA, 588 bp, synthetic construct
  source 1..588: mol_type = other DNA
atggaaactgatacattgctgctctgggttttgctgctctgggtgcctgggagtacaggcgacatgaggaggcagttcgaggatctcgttaaggatattacccttaataaggaggagaagaaagaaaactcttttgagatgcaacgaggggacgaagatcctcagatcgctgctcacgtggtctctgaagctaacagcaacgccgcttctgtcctccagtgggccaagaaaggttattacaccatgaaatcaaaccttgtaatgcttgaaaacgggaaacagcttacagtgaagagggaaggtctttactacgtctatacccaggtaaccttctgctcaaacagagaaccatcaagccagaggccattcatagtggggctctggctcaaaccttccagtggcagcgagagaatcttgttgaaagctgctaatacacatagtagtagccagctttgcgagcaacagtcagtccacctcgggggggtgtttgagttgcaagcaggggcctcagtattcgtgaatgtcactgaggcttcccaggtaattcacagggtaggctttagttcattcggtttgctgaagctttag

SEQ ID NO: 93 - DNA, 849 bp, synthetic construct
  source 1..849: mol_type = other DNA
atgcgaagaatgcagcttctgctccttattgctctgagtctcgcccttgtcaccaactccggggacagaatgaaacaaatcgaggacaaaattgaagaaatactgagtaaaatatatcacatcgaaaacgaaattgcacgcattaagaaattgattggcgaacgcaccagtggcggctctggtggcaccggaggttcaggcgggaccgggggctctgacaaagtcgaagaggaggttaaccttcatgaggactttgtgttcatcaagaagctgaaacggtgcaataaaggagaaggttctttgagcctccttaattgcgaagagatgcgacgacagttcgaggatctggttaaggacattacacttaataaggaagagaaaaaggagaactctttcgaaatgcagcgcggcgatgaagatccccagatagccgcccatgtcgtctctgaggccaactctaacgcagcatccgtcctccagtgggctaagaaaggatattatactatgaaaagcaatttggtcatgctcgaaaacggtaaacagctcactgttaagagagaaggcctctattacgtatatactcaagtaactttctgttctaatagggaaccctcctctcaaagaccttttatcgtaggactctggttgaaaccaagtagcggtagtgaaaggattctgctcaaagcagctaatactcactccagcagtcaactgtgcgaacaacaaagcgttcacctcgggggcgtctttgaacttcaggcaggtgccagtgttttcgtcaacgtaacagaagcatcccaggtaattcatcgagtagggttttctagctttggtttgctgaagctgtag

SEQ ID NO: 94 - DNA, 2160 bp, synthetic construct
  source 1..2160: mol_type = other DNA
atggaaactgatcgcctgttgctctgggtacttcttctgtgggtgcctgggtccactggtgacactgtacttacacaatcacccgctttggccgtttctcctggtgaacgggtcacaattagttgccgagcttccgattctgtatctactcttatgcattggtatcaacaaaaacctggtcagcagccaaaattgctcatttatcttgctagtcacttggagtccggcgtacctgctcgattcagcggtagtgggtctggcacagatttcactttgaccatagatcccgtggaggccgatgacactgcaacctactattgccagcaatcctggaacgacccttggactttcggcggcggcaccaagctggaactcaagcgagcagatgctgccccaaccgttagtatattcccaccctcaaccgaacaactcgccacaggaggcgctagtgtcgtgtgtcttatgaacaatttctatccacgagacattagcgtcaagtggaaaattgatgggacagaaaggcgagatggagttttggattcagtaacagaccaggattcaaaggattctacctatagcatgagctccaccttgagcctgaccaaagctgattatgaatctcataacctgtatacttgtgaagtggtgcataagacttctagctcaccagtggttaaatcttttaaccgcaacgaatgtcggcgcaagaggggttccggagagggaaggggtagtctgctcacctgcggcgatgttgaagaaaatcctggtcccatggacattcggctctctttggtattcctggtactttttataaagggggtgcaatgtgaagtccagctcgtggaaagcggtgggggcctggttcagcccggtcgcagccttaaacttagttgcgcagcatccggatttacattttctgactataacatggcctgggttcgacaggcacccaaaaaagggctggagtgggtcgcaactatcatatacgatggttcccggacatactatagagattcagtgaaggggcgctttacaataagcagggacaatgctaagtctaccttgtatcttcagatggactccctgaggagcgaagatacagcaacatattattgtgctacaaaccgctggttgctgcttcattatttcgactactggggtcagggcgtcatggtaactgtatcaagcgccgagaccacagccccttctgtatatccattggcaccaggtactgctctgaaatccaactcaatggtaacccttggatgtctggttaagggttattttcccgagcccgtcacagttacttggaactctggggccctttctagcggagtccatacctttcccgccgttttgcagagtggtctgtacacccttacctcaagcgtcacagttccatctagcacatggagctcccaggcagtaacttgtaatgtggcccatccagcctcctcaactaaggtagataaaaagatcgttcccagagaatgcaatccatgtggatgcaccgggtctgaggtcagcagtgtgttcattttcccacccaagactaaagatgtattgactattactcttacacccaaagtaacctgcgtggtggttgatattagtcaaaatgatcccgaggtacggttctcttggtttatcgacgacgtcgaagtacatacagctcagacacacgctcccgagaaacaaagcaattccactcttaggagcgtgtccgagttgccaatcgtacatagggattggcttaatggcaagacctttaagtgtaaggtcaattcaggggcattccccgcaccaatagagaagagtataagcaaacccgaggggacacccagaggtccacaggtctatacaatggctccccccaaggaagagatgacccaaagtcaagtctcaattacatgtatggtgaagggcttttatccacccgacatatacactgagtggaagatgaatggacagccccaagagaattataaaaacactccccctaccatggacaccgacgggtcctattttctttatagtaaattgaacgtgaaaaaggagacctggcaacaaggcaacactttcacctgctccgttcttcacgagggcctgcataatcatcataccgaaaagtctctcagtcattctccaggtaagtag

SEQ ID NO: 95 - DNA, 528 bp, Homo sapiens
  source 1..528: mol_type = other DNA
atggaaacagatacgttgctgttgtgggtacttctcctttgggtccctggcagcacaggggacgagaatagtttcgaaatgcagaagggcgaccagaacccacagatcgcggctcacgttatatcagaagcaagtagtaagaccacttccgtacttcagtgggctgaaaaaggatattacaccatgtccaacaatctcgtgacactggagaacggtaaacaacttacggtgaaacgacagggcctctattacatctacgctcaggtgacattctgctcaaatagggaggcttctagtcaagcgcccttcatcgccagcctgtgcctcaaatctcccggccggttcgaacgaatcctgttgcgagcggccaatacccatagctcagctaaaccttgcggccagcagagtattcatcttggtggtgtgtttgaacttcagccgggagcatctgtgttcgtcaacgtaacggaccctagccaagtgtctcatgggacaggttttacatccttcggactcctcaagttgtag

SEQ ID NO: 96 - DNA, 540 bp, Homo sapiens
  source 1..540: mol_type = other DNA
atgacagttctcgcgccagcttggagtcccaccacatacttgcttttgcttctgcttctgtcctctggcctgagtgggacccaagattgttcctttcaacattccccaattagttctgattttgcagtgaagattagagagctctcagactatctgctgcaagattatcctgtcacagtcgcttcaaacctgcaagacgaagagctctgcggtgccttgtggcggttggtcttggctcaaagatggatggagagactgaaaaccgtagcaggcagcaagatgcagggtctcctggaaagggtgaacacggaaatccattttgtgaccaagtgcgcgttccagcccccaccgagttgtctccggtttgttcaaacgaatatatcccggttgctccaggaaacctcagaacaactggtggctttgaaaccctggatcacaagacaaaactttagtcggtgcctcgaactccagtgccaaccagattcttctacacttccccccccgtggtccccgcgcccgttggaagcaacggccccatag

SEQ ID NO: 97 - DNA, 876 bp, Homo sapiens
  source 1..876: mol_type = other DNA
atggcctggagtcctctgtttctgactcttataactcactgtgccggcagttgggctataccccctcatgtacagaagtctgtaaacaacgacatgattgtaaccgacaataatggcgcagtgaaattcccacaactgtgtaagttctgtgatgtacggtttagtacatgcgacaatcaaaaaagctgtatgtctaactgctctattacatccatatgtgaaaaacctcaggaggtgtgtgttgccgtttggcgaaaaaatgatgagaatatcacactggagacagtatgtcatgaccctaaactgccataccatgatttcatactggaggacgccgccagtcctaagtgcattatgaaagagaaaaagaaacccggtgaaacattctttatgtgctcttgtagctctgacgagtgtaacgacaacattatattcagcgaggagtacaatacaagcaaccccgatataccacctcacgtacaaaaaagtgtcaacaacgatatgattgttaccgacaataacggagctgttaagtttcctcagttgtgcaagttctgcgatgtacgattctctacctgcgacaaccaaaagtcatgtatgtctaactgttccataacctccatctgcgagaagccccaggaagtctgcgtcgccgtgtggcggaaaaacgacgagaatatcactcttgaaaccgtttgtcatgatcctaaactgccctatcacgactttattctggaagatgctgcttcccctaagtgtatcatgaaagaaaagaagaaacctggggagacattctttatgtgttcatgctcctccgatgagtgtaacgacaatatcatcttctctgaggaatacaacacttctaaccctgattag

SEQ ID NO: 98 - DNA, 2178 bp, Homo sapiens
  source 1..2178: mol_type = other DNA
atggcctggtcccctctttttctgaccctcatcacacactgtgcaggctcatgggctgagaccgtcttgacccagtccccaggaactttgtctctgtctcctggtgaaagagctacccttagttgtcgagcctctcagtcccttggttctagctatctcgcttggtaccagcaaaagccaggccaggccccacgactgctgatctacggagcatcttcacgggctcccggcattcccgatcgattttccggatctggtagtggtacagatttcacactgaccatatctcgcctggagcccgaggactttgctgtttattattgtcagcagtacgccgattctcctatcacttttggacagggaacccgcctggagattaagcgcacagtagcagctccatccgtctttatctttccaccatcagatgaacagctcaagagtgggaccgcaagtgtagtatgcctgctgaacaatttttaccctagagaggccaaagtgcagtggaaggtggataacgccctccagagtggcaatagtcaagaaagtgttactgagcaagatagtaaggactctacatactctttgagttctactttgaccctgtcaaaagcagattatgaaaaacataaggtgtatgcatgtgaagttacacaccaagggttgtcctctccagttacaaaatcttttaatagaggagagtgccgccgcaaacgcggtagtggagaaggtcgaggctcactcttgacctgtggcgacgtggaagaaaatcccggtcctatggattggacttggagggtattttgtcttttggcagtaacacctggagctcacccccaagtacagctcgtccaatctggtgccgaggttaaaaagcctggaagttcagtgaaggtctcttgcaaggcatctggatacaccttttcatctaacgtcatatcctgggtacggcaagccccaggacagggacttgagtggatgggaggggtcatccccatcgtggacattgctaattacgctcagcgattcaaagggcgggttactataactgccgacgagtctacctcaactacctacatggagttgtcctctctccgctccgaggacactgctgtatattactgtgccagcactctcgggttggtgttggatgccatggactattggggacaaggaaccctggtgacagttagctccgcaagcactaaaggcccttctgtttttcccttggcaccttgtagtaggtctacctctgagtctacagcagcacttggatgcttggttaaggactattttcccgagccagttacagtctcttggaacagtggtgccctcacaagtggggttcataccttccccgcagtcctccagagtagtggcctttacagcctctcatcagttgtgactgttcctagttcatcactcggtactaagacatatacatgtaacgtagaccacaagccaagcaacacaaaagtagacaaacgagtcgaatctaagtatggacccccttgtccctcctgtcctgctcccgagttccttgggggcccttccgtgttcttgtttcctcccaagcccaaggataccctcatgatctcacgaaccccagaggtaacatgtgtggttgttgacgtaagtcaggaagatcccgaagtgcaatttaattggtacgtggatggcgtcgaagtccataacgctaaaacaaaaccccgagaggaacaattcaattccacatatcgggtggtgagtgtattgaccgttcttcaccaagattggctgaacggcaaggagtataagtgtaaagtaagcaacaaaggtctgccaagtagcatagaaaaaacaatatctaaagctaagggccaaccaagggaaccacaagtatatacattgcccccctctcaggaagagatgacaaagaatcaagttagcctgacctgtttggtaaaggggttctatccctcagatatagcagtcgagtgggaatctaacggccagcccgagaataattataaaacaaccccccctgtgttggactcagacggcagcttctttctctattcacggctcactgttgataagtcccgatggcaggaggggaatgttttcagctgtagcgtgatgcacgaagctctccacaaccactatacacagaaaagtttgtctttgtcccttggaaaatag

SEQ ID NO: 99 - DNA, 1062 bp, Homo sapiens
  source 1..1062: mol_type = other DNA
atgagtacatcctttccagagctggatctggagaattttgagtatgacgacagtgccgaagcctgctacctcggggacatagtcgcattcgggacaatctttttgtctgtattttacgccctggtgtttacatttggcctggttggaaatctgttggtcgtactcgctctcaccaattcccgaaaacccaaaagtataacagacatatacctgttgaatctggcactgagtgaccttttgttcgtcgccacccttcctttttggacacactaccttatcagtcacgaggggcttcataatgctatgtgcaagctcactactgccttcttctttatcggattcttcgggggtatcttttttatcacagttattagcattgaccgataccttgccatagtgctcgcagccaactcaatgaacaaccgcaccgtgcagcatggagtgactatttccttgggtgtgtgggccgctgctatacttgtcgccagccctcaattcatgtttaccaaaaggaaagacaatgagtgcctcggagattaccctgaggtgttgcaagaaatgtggcctgtacttcgaaatagcgaagtgaatatactcggctttgctcttcctctgctcatcatgtcattctgttattttcgaataatccaaacattgttcagctgtaagaaccgaaagaaagcccgcgccgtacgcctgattctgctcgttgtgttcgccttttttctgttttggactccttacaacataatgatattcctggagactctcaaattctataacttttttccctcctgtgatatgaaaagggaccttagattggctctcagtgtcactgaaacagtagcctttagccattgttgtctcaaccctttcatatatgcatttgcaggggaaaagttccggcggtatctcggacatttgtatcggaagtgcttggccgtgttgtgtggtcatcctgtccataccggattctctcctgagagtcaacggagccgccaagattcaatcctgtccagtttcactcactatacttcagagggggatggcagccttctgctc

SEQ ID NO: 100 - DNA, 1458 bp, synthetic construct
  source 1..1458: mol_type = other DNA
atggagaccgacactttgttgctgtgggtacttttgttgtgggtcccaggatctaccggggatatggaaccctctcctcttgaactgccagtagacgccgtgcgccgcattgcagccgagttgaattgcgatccaacagatgaacgcgttgccctgaggctcgacgaagaggataaattgtcacatttcaggaactgcttttacattccaaagatgagggatcttccatccatagatcttagcctcgtgtccgaggatgacgatgccatatattttcttgggaacagtcttgggttgcagccaaaaatggtacggacatatctcgaagaggagctggacaaatgggctaaaatgggtgcttacggccacgacgtgggaaaacgcccctggatagttggcgacgaatctatcgtgagtcttatgaaagatatagttggagcacatgagaaagaaattgcactgatgaatgcccttactatcaatctgcatctcctcttgctttcattctttaagcccactcctaaacgccacaaaatacttttggaagcaaaagcctttccaagcgaccactacgctattgagtcacaaatacaactccatggacttgatgtggaaaagtctatgcggatggtaaaaccacgcgaaggcgaggagacccttcgaatggaggacatacttgaggtcatcgaagaagaaggagatagtatagcagttatccttttcagcgggctgcacttctacacaggtcaactctttaacattccagctattactaaggcaggccacgctaaaggatgcttcgtgggctttgaccttgcacacgcagtaggaaacgtagagctccgcttgcacgattggggcgttgatttcgcctgctggtgttcatataagtatcttaactcaggagctggtgggttggcaggcgcattcgtacacgagaaacacgctcataccgtaaagcctgcactggtagggtggttcggacacgatctctctacccgcttcaatatggataataaactccagcttatacctggcgccaatggattcaggatctcaaatcctcctattttgctcgtttgcagtttgcacgcatctcttgaggtgttccagcaggctaccatgactgcactccgccggaagtcaatccttttgaccggatacttggagtatatgctgaaacattatcactcaaaagataacactgagaataagggccccatagtaaacattatcactccatctcgggctgaagagcgcggctgccaactcacattgactttttccattcccaagaagtcagtgttcaaagagttggagaaacggggggttgtatgtgataagcgggagccagatggaatccgcgttgccccagtccccctctataattcttttcacgatgtatacaagtttattagactgctgacaagtatcttggactcatctgagcgatcttag

SEQ ID NO: 101 - DNA, 1395 bp, synthetic construct
  source 1..1395: mol_type = other DNA
atggaaccctctcctcttgaactgccagtagacgccgtgcgccgcattgcagccgagttgaattgcgatccaacagatgaacgcgttgccctgaggctcgacgaagaggataaattgtcacatttcaggaactgcttttacattccaaagatgagggatcttccatccatagatcttagcctcgtgtccgaggatgacgatgccatatattttcttgggaacagtcttgggttgcagccaaaaatggtacggacatatctcgaagaggagctggacaaatgggctaaaatgggtgcttacggccacgacgtgggaaaacgcccctggatagttggcgacgaatctatcgtgagtcttatgaaagatatagttggagcacatgagaaagaaattgcactgatgaatgcccttactatcaatctgcatctcctcttgctttcattctttaagcccactcctaaacgccacaaaatacttttggaagcaaaagcctttccaagcgaccactacgctattgagtcacaaatacaactccatggacttgatgtggaaaagtctatgcggatggtaaaaccacgcgaaggcgaggagacccttcgaatggaggacatacttgaggtcatcgaagaagaaggagatagtatagcagttatccttttcagcgggctgcacttctacacaggtcaactctttaacattccagctattactaaggcaggccacgctaaaggatgcttcgtgggctttgaccttgcacacgcagtaggaaacgtagagctccgcttgcacgattggggcgttgatttcgcctgctggtgttcatataagtatcttaactcaggagctggtgggttggcaggcgcattcgtacacgagaaacacgctcataccgtaaagcctgcactggtagggtggttcggacacgatctctctacccgcttcaatatggataataaactccagcttatacctggcgccaatggattcaggatctcaaatcctcctattttgctcgtttgcagtttgcacgcatctcttgaggtgttccagcaggctaccatgactgcactccgccggaagtcaatccttttgaccggatacttggagtatatgctgaaacattatcactcaaaagataacactgagaataagggccccatagtaaacattatcactccatctcgggctgaagagcgcggctgccaactcacattgactttttccattcccaagaagtcagtgttcaaagagttggagaaacggggggttgtatgtgataagcgggagccagatggaatccgcgttgccccagtccccctctataattcttttcacgatgtatacaagtttattagactgctgacaagtatcttggactcatctgagcgatcttag

SEQ ID NO: 102 - DNA, 573 bp, synthetic construct
  source 1..573: mol_type = other DNA
atgaatttcttgctgagctgggtgcattggacactcgcattgttgctgtacttgcaccatgccaagtggtcccaggctgcacccactactgagggcgagcaaaagtctcatgaggtgattaaatttatggacgtttaccaacgatcatactgtcggccaatcgaaaccctcgtagatatattccaggagtacccagacgagatcgaatacattttcaagccctcatgtgtcccattgatgcgatgtgctgggtgctgtaacgacgaagcacttgaatgtgtccccacctccgagagtaacatcacaatgcaaataatgagaatcaagccccaccaatcccaacatatcggtgaaatgtcattccttcagcattcccgctgcgagtgccggcctaagaaggaccgcaccaaaccagagaaccattgtgaaccctgttctgagagacggaagcacttgttcgtacaggaccctcaaacatgcaagtgcagctgtaagaataccgactcacggtgtaaagctaggcaactggagcttaatgaaaggacctgccgatgcgataaacccaggaggtaa

SEQ ID NO: 103 - DNA, 426 bp, synthetic construct
  source 1..426: mol_type = other DNA
atgtggttgcagaatttgctcttcctggggattgtggtctacagcctctccgcacctacccgctctcctatcacagttacaagaccctggaaacatgtggaggccattaaagaagcattgaatttgttggacgatatgcccgtcaccctgaatgaagaagtagaagttgtttctaatgagttcagctttaaaaaattgacctgtgtgcagacacggcttaaaatttttgaacagggacttagaggaaactttactaagctgaagggggcacttaacatgacagcttcttattatcagacctattgtcctccaacacctgaaaccgactgtgaaacacaggtaaccacttacgccgattttattgattctttgaaaacattcctcaccgatataccatttgagtgtaagaagccaggccaaaagtag

SEQ ID NO: 104 - DNA, 822 bp, synthetic construct
  source 1..822: mol_type = other DNA
atggaaactgacacacttcttctgtgggtcttgctcctgtgggtcccaggctctactggtgacagtcctgataggccatggaacccacctacctttagtccagccttgctcgtcgtaaccgaaggggacaacgctacattcacctgctcttttagcaatacttctgagagttttcatgtagtctggcatcgggagagtccatccggacaaacagatactttggccgcttttccagaggataggtctcaacctgggcaagacgcaaggtttcgagtcacacagcttcctaacgggagagattttcacatgtctgtagttcgggcacgccgaaatgattctggcacatatgtttgcggtgtgatctcacttgctccaaagattcaaataaaggagagccttcgcgccgagttgcgggtgactgagcgggagcccaagtcctgcgacaaaacccatacttgtccaccctgtggcggcgggtcatccggtggcgggtctggggggcaaccaagagagccacaggtatatactcttccccccagcagagaagaaatgacaaaaaaccaagtgtccctgacatgtctggttaaaggattttatcccagtgacattgctgtagaatgggaatccaatggtcaacccgagaataactacaaaaccactcctccagtattggacagtgacggttccttcttcctctattccaaacttacagtggataaatcccgctggcagcaagggaatgtattcagctgtagtgtcatgcacgaagctcttcataaccattatacacagaaatctctttccctgagcccaggtaaatag

SEQ ID NO: 105 - DNA, 1116 bp, Mus musculus
  source 1..1116: mol_type = other DNA
atggagactgatacacttttgctctgggttttgctcttgtgggtaccagggtctactggagatgcacaaactcctgcattcaacaagcctaaggtagagcttcatgtccatttggacggagccataaaacctgaaaccatactctatttcggcaagaaacggggtatagcacttcccgctgataccgtggaagagttgagaaatatcattggcatggacaaacctcttagcctgcctggctttcttgcaaagttcgactactatatgccagttatagcagggtgtagagaagcaataaagcgaatcgcctatgagttcgttgagatgaaggctaaagaaggagttgtttacgtggaagtccggtactcacctcatctgcttgctaatagcaaggtggacccaatgccatggaatcaaactgaaggtgatgtaacccctgacgatgtggtcgatttggtcaatcaaggtctccaagaaggcgagcaggctttcggcattaaggtaagaagtatattgtgctgtatgcgacatcaaccttcatggtccctggaggtcctcgaattgtgcaaaaagtacaatcaaaaaacagtggtcgcaatggatctcgctggagatgagaccatagaaggttcctctcttttccccggtcatgtcgaagcatatgaaggggctgtcaaaaatggtatccaccgcaccgtccacgcaggggaagtagggtccccagaagtagtcagggaagccgttgacattttgaaaacagaaagagtcgggcatggctaccatacaatagaggacgaagccttgtacaatcgacttttgaaagaaaatatgcacttcgaggtctgtccctggagttcatatctcaccggagcatgggaccccaaaacaacccacgccgtcgtacgcttcaagaatgataaggcaaactacagtttgaatacagatgatccactgatattcaagtcaacacttgacactgactaccagatgacaaaaaaagatatgggtttcaccgaagaagagttcaagagattgaacattaacgcagcaaaaagctccttcctgccagaggaagagaaaaaagaattgcttgaaaggttgtatcgagaataccaa

SEQ ID NO: 106 - DNA, 1056 bp, Mus musculus
  source 1..1056: mol_type = other DNA
atggcacaaactccagcttttaataagcccaaagtggaacttcatgttcatctggatggggcaattaagcccgaaactatattgtactttggcaaaaagaggggtattgccctgccagcagataccgttgaggagcttcgcaacatcattgggatggacaagcccctctctctgccaggttttctcgctaaattcgattattatatgcctgttattgctggttgccgggaggccatcaagaggatagcctacgagtttgttgagatgaaggccaaagagggcgtggtgtacgtagaggtcagatacagccctcacctgcttgccaacagcaaggtggacccaatgccctggaaccaaaccgagggggatgtcactcccgacgacgttgtagacctcgtaaatcagggccttcaagagggcgagcaggcatttggcataaaagtccggtctatactctgctgtatgaggcaccaaccctcctggtctttggaggtacttgagttgtgtaagaaatacaatcaaaagactgtagtcgccatggatcttgcaggcgatgaaaccatcgagggtagctccttgttccctggacatgttgaagcctacgagggggccgtaaaaaatgggatacacaggactgtccacgctggtgaagtcggaagcccagaggtggtaagggaggcagttgacatactcaagacagagcgggttggacacggataccacacaattgaggacgaggccctgtataaccgcctcctcaaagagaacatgcattttgaggtgtgtccttggtccagctacctgactggtgcttgggaccctaaaacaactcacgccgtggtccggttcaagaacgataaagccaattactctttgaataccgacgaccccctcatattcaaatcaacattggataccgactaccaaatgaccaaaaaggatatggggtttactgaagaggagttcaagaggctcaacataaatgccgctaaatcctcctttctccccgaggaagaaaaaaaagaactccttgagcggctgtatagggagtatcaa

SEQ ID NO: 107 - DNA, 966 bp, Mus musculus
  source 1..966: mol_type = other DNA
atggaaacagatacactcttgctctgggtactgcttctgtgggtccccggctctactggggatgaagatgatgtaactactacagaagaactcgctcccgctcttgtccccccacccaagggtacctgcgccggttggatggctggcatcccaggacatccaggtcacaacggtacccccggaagagatggtcgggatggaactcccggcgagaagggcgaaaaaggggatgcagggcttctgggacctaaaggtgaaacaggggacgttggaatgactggtgcagaagggcctcgcggctttcctggcacccctgggaggaaaggagagcccggagagctccagagaactgaacctcggcctgcactcactataactacttcccctaatcttgggacccgcgagaacaacgccgatcaggttacacctgtaagccatatcgggtgccccaatactacccagcaagggagtcccgtgttcgcaaagcttttggctaaaaaccaagcatccctgtgtaacactactcttaattggcattcacaagacggtgctggtagctcttatctttctcaggggctgcggtacgaagaagataagaaggaattggttgtggattctccaggactctattatgtctttctcgaattgaagctcagtcccaccttcacaaacactggacacaaagtccagggctgggtaagtctggtactccaagcaaagccccaggttgacgatttcgacaatttggcactcaccgtagagcttttcccatgctccatggaaaataaacttgttgatcggtcatggtcacagctcttgctgcttaaggcagggcatcgcctctcagtgggtctgagagcttatttgcatggtgcacaagatgcttacagggattgggaattgtcctacccaaacactacaagtttcgggttgttccttgtcaaacctgataacccatgggagtag

SEQ ID NO: 108 - DNA, 900 bp, Mus musculus
  source 1..900: mol_type = other DNA
atggaaactgatacactcctcctgtgggtccttcttttgtgggtgcccggatcaaccggcgatggctggatggcaggcatcccaggacacccaggacacaacggtactccaggtcgagacggtcgggatgggactcctggggagaaaggcgagaaaggggacgctggtttgctcggtcctaagggggaaaccggggatgtaggaatgacaggggctgaagggcctcggggatttcctgggacaccaggcaggaagggtgaaccaggggaggccctccagcgcaccgagccacggccagctctgaccataacaacaagtccaaacctgggcacacgcgaaaacaatgctgaccaggtgactcctgtaagtcacatcggatgccctaacactacacaacagggctctcctgtatttgcaaagcttctcgcaaaaaatcaagcatcactttgtaatacaaccctgaactggcattctcaggacggagcagggtcctcttatttgtctcaagggctccgctacgaagaagataaaaaggaattggttgttgacagtccaggtttgtattatgtgtttttggaacttaagctgtcaccaaccttcactaacaccggccacaaggtccaaggctgggttagtcttgttttgcaagccaaacctcaagtggatgattttgacaatctggctttgactgttgagctttttccatgcagtatggagaataaactggttgatcggtcatggtcacagctccttctgctcaaggccggacataggctgagtgtgggacttcgggcctacttgcacggcgcccaggacgcataccgagactgggaactcagctaccctaacacaacttcttttgggttgttccttgtcaaacccgataatccttgggaatag

SEQ ID NO: 109 - DNA, 870 bp, Mus musculus
  source 1..870: mol_type = other DNA
atggagactgatactttgctcctgtgggttcttctcctgtgggttcctggttccacaggggatatgcatgtcaatggcaaggtagcactcgtgactggggctgcacagggtatcgggaaagcttttgccgaggccctgttgctgcatggcgccaaggtcgctttggtagattggaacttggaggctggagttaaatgcaaagctgcactcgacgaacaatttgagcctcaaaaaaccctctttgtgcagtgtgacgttgctgaccaaaagcaactcagggacacattcaggaaggtcgtagaccatttcggacgcctcgatatactcgttaataatgccggggtaaacaacgaaaagaactgggaacaaacattgcaaatcaacctggtaagtgtcattagcggaacttatctgggtcttgattatatgagcaagcagaacgggggcgagggcgggatcattatcaacatgtcaagtcttgccggattgatgccagttgctcagcagcctgtttactgtgccagcaagcacggtattattgggtttacccggagtgccgccatggccgcaaatcttatgaagagtggggtaagactgaatgttatctgcccaggtttcgtagataccccaatcctggagagcatcgagaaggaggaaaatatgggacaatacattgaatataaagatcaaatcaaggctatgatgaagttctacggggttctgcatccatccacaattgccaacgggctcattaatctgattgaggacgacgccttgaacggagctataatgaaaatcacagcttccaaaggcattcacttccaagattatgatatatcacccttgcttgtcaaggctcctctgacaagt

SEQ ID NO: 110 - DNA, 807 bp, Mus musculus
  source 1..807: mol_type = other DNA
atgcatgtcaatggcaaggtagcactcgtgactggggctgcacagggtatcgggaaagcttttgccgaggccctgttgctgcatggcgccaaggtcgctttggtagattggaacttggaggctggagttaaatgcaaagctgcactcgacgaacaatttgagcctcaaaaaaccctctttgtgcagtgtgacgttgctgaccaaaagcaactcagggacacattcaggaaggtcgtagaccatttcggacgcctcgatatactcgttaataatgccggggtaaacaacgaaaagaactgggaacaaacattgcaaatcaacctggtaagtgtcattagcggaacttatctgggtcttgattatatgagcaagcagaacgggggcgagggcgggatcattatcaacatgtcaagtcttgccggattgatgccagttgctcagcagcctgtttactgtgccagcaagcacggtattattgggtttacccggagtgccgccatggccgcaaatcttatgaagagtggggtaagactgaatgttatctgcccaggtttcgtagataccccaatcctggagagcatcgagaaggaggaaaatatgggacaatacattgaatataaagatcaaatcaaggctatgatgaagttctacggggttctgcatccatccacaattgccaacgggctcattaatctgattgaggacgacgccttgaacggagctataatgaaaatcacagcttccaaaggcattcacttccaagattatgatatatcacccttgcttgtcaaggctcctctgacaagt

SEQ ID NO: 111 - skipped

SEQ ID NO: 112 - protein, 22 aa, Homo sapiens
MCHQQLVISWFSLVFLASPLVA

SEQ ID NO: 113 - protein, 14 aa, synthetic construct
MQLLSCIALILALV

SEQ ID NO: 114 - protein, 20 aa, Homo sapiens
MYRMQLLSCIALSLALVTNS

SEQ ID NO: 115 - protein, 15 aa, Homo sapiens
MNLLLILTFVAAAVA

SEQ ID NO: 116 - protein, 17 aa, unidentified
  source 1..17: note = Gaussia Luciferase
MGVKVLFALICIAVAEA

SEQ ID NO: 117 - protein, 24 aa, Homo sapiens
MPMGSLQPLATLYLLGMLVASCLG

SEQ ID NO: 118 - protein, 21 aa, Mus musculus
METDTLLLWVLLLWVPGSTGD

SEQ ID NO: 119 - protein, 22 aa, Homo sapiens
MDMRVPAQLLGLLLLWLRGARC

SEQ ID NO: 120 - protein, 16 aa, synthetic construct
MKCLLYLAFLFIGVNC

SEQ ID NO: 121 - protein, 28 aa, Homo sapiens
MNIKGSPWKGSLLLLLVSNLLLCQSVAP

SEQ ID NO: 122 - protein, 18 aa, Homo sapiens
MKWVTFISLLFLFSSAYS

SEQ ID NO: 123 - protein, 19 aa, synthetic construct
MTRLTVLALLAGLLASSRA

SEQ ID NO: 124 - protein, 17 aa, Homo sapiens
MRAWIFFLLCLAGRALA

SEQ ID NO: 125 - protein, 16 aa, Homo sapiens
MPLLLLLPLLWAGALA

SEQ ID NO: 126 - protein, 29 aa, Homo sapiens
MNSFSTSAFGPVAFSLGLLLVLPAAFPAP

SEQ ID NO: 127 - protein, 20 aa, Homo sapiens
MTSKLAVALLAAFLISAALC

SEQ ID NO: 128 - protein, 23 aa, Homo sapiens
MKVSAALLCLLLIAATFIPQGLA

SEQ ID NO: 129 - protein, 26 aa, Homo sapiens
MGAAARTLRLALGLLLLATLLRPADA

SEQ ID NO: 130 - protein, 21 aa, Homo sapiens
MSPLLRRLLLAALLQLAPAQA

SEQ ID NO: 131 - protein, 21 aa, Homo sapiens
MNNLLCCALVFLDISIKWTTQ

SEQ ID NO: 132 - protein, 23 aa, Homo sapiens
MQMSPALTCLVLGLALVFGEGSA

SEQ ID NO: 133 - protein, 34 aa, Homo sapiens
MARAALSAAPSNPRLLRVALLLLLLVAAGRRAAG

SEQ ID NO: 134 - protein, 21 aa, Homo sapiens
MNAKVVVVLVLVLTALCLSDG

SEQ ID NO: 135 - protein, 22 aa, Homo sapiens
MERIVICLMVIFLGTLVHKSSS

SEQ ID NO: 136 - protein, 21 aa, synthetic construct
MALPVTALLLPLALLLHAARP

SEQ ID NO: 137 - protein, 21 aa, synthetic construct
MALPVTALLLPLALLLHAARP

SEQ ID NO: 138 - protein, 21 aa, synthetic construct
MLLVTSLLLCELPHPAFLLIP

SEQ ID NO: 139 - DNA, 63 bp, synthetic construct
  source 1..63: mol_type = other DNA
atggccttaccagtgaccgccttgctcctgccgctggccttgctgctccacgccgccaggccg

SEQ ID NO: 140 - DNA, 63 bp, synthetic construct
  source 1..63: mol_type = other DNA
atggcgctcccggtgacagcacttctcttgcctcttgccctgctgttgcatgccgcgcgccca

SEQ ID NO: 141 - DNA, 63 bp, synthetic construct
  source 1..63: mol_type = other DNA
atgttgctcgtgacatccctcttgctttgtgagttgcctcatcccgcattcctgctcatccca

SEQ ID NO: 142 - protein, 10 aa, unidentified
  source 1..10: note = Hepatitis C virus
DEMEECSQHL

SEQ ID NO: 143 - protein, 9 aa, unidentified
  source 1..9: note = Hepatitis C virus
EDVVPCSMG

SEQ ID NO: 144 - protein, 99 aa, Human immunodeficiency virus 1
PQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNF

SEQ ID NO: 145 - protein, 19 aa, synthetic construct
EDANSEPLFAERKDABCYL

SEQ ID NO: 146 - protein, 4 aa, synthetic construct
YVAD

SEQ ID NO: 147 - protein, 5 aa, synthetic construct
VDVAD

SEQ ID NO: 148 - protein, 4 aa, synthetic construct
DEVD

SEQ ID NO: 149 - protein, 4 aa, synthetic construct
VEHD

SEQ ID NO: 150 - protein, 4 aa, synthetic construct
LGHD

SEQ ID NO: 151 - protein, 5 aa, synthetic construct
LQTDG

SEQ ID NO: 152 - protein, 8 aa, synthetic construct
EVNLDAEF

SEQ ID NO: 153 - protein, 7 aa, synthetic construct
PQGIAGQ

SEQ ID NO: 154 - protein, 7 aa, unidentified
  source 1..7: note = Tobacco Etch virus
ENLYFQS

SEQ ID NO: 155 - protein, 6 aa, synthetic construct
  MOD_RES 1: note = Abz is linked to Histidine
  MOD_RES 6: note = Lys(Dnp)
HPFHLK

SEQ ID NO: 156 - protein, 1306 aa, synthetic construct
MGAASGRRGPGLLLPLPLLLLLPPQPALALDPGLQPGNFSADEAGAQLFAQSYNSSAEQVLFQSVAASWAHDTNITAENARRQEEAALLSQEFAEAWGQKAKELYEPIWQNFTDPQLRRIIGAVRTLGSANLPLAKRQQYNALLSNMSRIYSTAKVCLPNKTATCWSLDPDLTNILASSRSYAMLLFAWEGWHNAAGIPLKPLYEDFTALSNEAYKQDGFTDTGAYWRSWYNSPTFEDDLEHLYQQLEPLYLNLHAFVRRALHRRYGDRYINLRGPIPAHLLGDMWAQSWENIYDMVVPFPDKPNLDVTSTMLQQGWNATHMFRVAEEFFTSLELSPMPPEFWEGSMLEKPADGREVVCHASAWDFYNRKDFRIKQCTRVTMDQLSTVHHEMGHIQYYLQYKDLPVSLRRGANPGFHEAIGDVLALSVSTPEHLHKIGLLDRVTNDTESDINYLLKMALEKIAFLPFGYLVDQWRWGVFSGRTPPSRYNFDWWYLRTKYQGICPPVTRNETHFDAGAKFHVPNVTPYIRYFVSFVLQFQFHEALCKEAGYEGPLHQCDIYRSTKAGAKLRKVLQAGSSRPWQEVLKDMVGLDALDAQPLLKYFQPVTQWLQEQNQQNGEVLGWPEYQWHPPLPDNYPEGIDLVTDEAEASKFVEEYDRTSQVVWNEYAEANWNYNTNITTETSKILLQKNMQIANHTLKYGTQARKFDVNQLQNTTIKRIIKKVQDLERAALPAQELEEYNKILLDMETTYSVATVCHPNGSCLQLEPDLTNVMATSRKYEDLLWAWEGWRDKAGRAILQFYPKYVELINQAARLNGYVDAGDSWRSMYETPSLEQDLERLFQELQPLYLNLHAYVRRALHRHYGAQHINLEGPIPAHLLGNMWAQTWSNIYDLVVPFPSAPSMDTTEAMLKQGWTPRRMFKEADDFFTSLGLLPVPPEFWNKSMLEKPTDGREVVCHASAWDFYNGKDFRIKQCTTVNLEDLVVAHHEMGHIQYFMQYKDLPVALREGANPGFHEAIGDVLALSVSTPKHLHSLNLLSSEGGSDEHDINFLMKMALDKIAFIPFSYLVDQWRWRVFDGSITKENYNQEWWSLRLKYQGLCPPVPRTQGDFDPGAKFHIPSSVPYIRYFVSFIIQFQFHEALCQAAGHTGPLHKCDIYQSKEAGQRLATAMKLGFSRPWPEAMQLITGQPNMSASAMLSYFKPLLDWLRTENELHGEKLGWPQYNWTPNSARSEGPLPDSGRVSFLGLDLDAQQARVGQWLLLFLGIALLVATLGLSQRLFSIRHRSLHRHSHGPQFGSEVELRHS

SEQ ID NO: 157 - protein, 619 aa, Dengue virus
SGVLWDTPSPPEVERAVLDDGIYRIMQRGLLGRSQVGVGVFQDGVFHTMWHVTRGAVLMYQGKRLEPSWASVKKDLISYGGGWRFQGSWNTGEEVQVIAVEPGKNPKNVQTAPGTFKTPEGEVGAIALDFKPGTSGSPIVNREGKIVGLYGNGVVTTSGTYVSAIAQAKASQEGPLPEIEDEVFRKRNLTIMDLHPGSGKTRRYLPAIVREAIRRNVRTLILAPTRVVASEMAEALKGMPIRYQTTAVKSEHTGKEIVDLMCHATFTMRLLSPVRVPNYNMIIMDEAHFTDPASIARRGYISTRVGMGEAAAIFMTATPPGSVEAFPQSNAVIQDEERDIPERSWNSGYEWITDFPGKTVWFVPSIKSGNDIANCLRKNGKRVIQLSRKTFDTEYQKTKNNDWDYVVTTDISEMGANFRADRVIDPRRCLKPVILKDGPERVILAGPMPVTVASAAQRRGRIGRNQNKEGDQYVYMGQPLNNDEDHAHWTEAKMLLDNINTPEGIIPALFEPEREKSAAIDGEYRLRGEARKTFVELMRRGDLPVWLSYKVASEGFQYSDRRWCFDGERNNQVLEENMDVEMWTKEGERKKLRPRWLDARTYSDPLALREFKEFAAGRR

SEQ ID NO: 158 - protein, 618 aa, Dengue virus
AGVLWDVPSPPPVGKAELEDGAYRIKQKGILGYSQIGAGVYKEGTFHTMWHVTRGAVLMHKGKRIEPSWADVKKDLISYGGGWKLEGEWKEGEEVQVLALEPGKNPRAVQTKPGLFKTNAGTIGAVSLDFSPGTSGSPIIDKKGKVVGLYGNGVVTRSGAYVSAIAQTEKSIEDNPEIEDDIFRKRKLTIMDLHPGAGKTKRYLPAIVREAIKRGLRTLILAPTRVVAAEMEEALRGLPIRYQTPAIRAEHTGREIVDLMCHATFTMRLLSPVRVPNYNLIIMDEAHFTDPASIAARGYISTRVEMGEAAGIFMTATPPGSRDPFPQSNAPIMDEEREIPERSWSSGHEWVTDFKGKTVWFVPSIKAGNDIAACLRKNGKKVIQLSRKTFDSEYVKTRTNDWDFVVTTDISEMGANFKAERVIDPRRCMKPVILTDGEERVILAGPMPVTHSSAAQRRGRIGRNPKNENDQYIYMGEPLENDEDCAHWKEAKMLLDNINTPEGIIPSMFEPEREKVDAIDGEYRLRGEARKTFVDLMRRGDLPVWLAYRVAAEGINYADRRWCFDGIKNNQILEENVEVEIWTKEGERKKLKPRWLDAKIYSDPLALKEFKEFAAGRK

SEQ ID NO: 159 - protein, 619 aa, Dengue virus
SGVLWDVPSPPETQKAELEEGVYRIKQQGIFGKTQVGVGVQKEGVFHTMWHVTRGAVLTHNGKRLEPNWASVKKDLISYGGGWRLSAQWQKGEEVQVIAVEPGKNPKNFQTMPGIFQTTTGEIGAIALDFKPGTSGSPIINREGKVVGLYGNGVVTKNGGYVSGIAQTNAEPDGPTPELEEEMFKKRNLTIMDLHPGSGKTRKYLPAIVREAIKRRLRTLILAPTRVVAAEMEEALKGLPIRYQTTATKSEHTGREIVDLMCHATFTMRLLSPVRVPNYNLIIMDEAHFTDPASIAARGYISTRVGMGEAAAIFMTATPPGTADAFPQSNAPIQDEERDIPERSWNSGNEWITDFVGKTVWFVPSIKAGNDIANCLRKNGKKVIQLSRKTFDTEYQKTKLNDWDFVVTTDISEMGANFKADRVIDPRRCLKPVILTDGPERVILAGPMPVTVASAAQRRGRVGRNPQKENDQYIFMGQPLNKDEDHAHWTEAKMLLDNINTPEGIIPALFEPEREKSAAIDGEYRLKGESRKTFVELMRRGDLPVWLAHKVASEGIKYTDRKWCFDGERNNQILEENMDVEIWTKEGEKKKLRPRWLDARTYSDPLALKEFKDFAAGRK

SEQ ID NO: 160 - protein, 618 aa, Dengue virus
SGALWDVPSPAATQKAALSEGVYRIMQRGLFGKTQVGVGIHIEGVFHTMWHVTRGSVICHETGRLEPSWADVRNDMISYGGGWRLGDKWDKEEDVQVLAIEPGKNPKHVQTKPGLFKTLTGEIGAVTLDFKPGTSGSPIINRKGKVIGLYGNGVVTKSGDYVSAITQAERIGEPDYEVDEDIFRKKRLTIMDLHPGAGKTKRILPSIVREALKRRLRTLILAPTRVVAAEMEEALRGLPIRYQTPAVKSEHTGREIVDLMCHATFTTRLLSSTRVPNYNLIVMDEAHFTDPSSVAARGYISTRVEMGEAAAIFMTATPPGTTDPFPQSNSPIEDIEREIPERSWNTGFDWITDYQGKTVWFVPSIKAGNDIANCLRKSGKKVIQLSRKTFDTEYPKTKLTDWDFVVTTDISEMGANFRAGRVIDPRRCLKPVILPDGPERVILAGPIPVTPASAAQRRGRIGRNPAQEDDQYVFSGDPLKNDEDHAHWTEAKMLLDNIYTPEGIIPTLFGPEREKTQAIDGEFRLRGEQRKTFVELMRRGDLPVWLSYKVASAGISYKDREWCFTGERNNQILEENMEVEIWTREGEKKKLRPKWLDARVYADPMALKDFKEFASGRK

SEQ ID NO: 161 - protein, 64 aa, Homo sapiens
LQMLPESEDEESYDTESEFTEFTEDELPYDDGSLQMLPESEDEESYDTESEFTEFTEDELPYDD

SEQ ID NO: 162 - protein, 57 aa, Homo sapiens
EIKDKEEVQRKRQKLMPNFSDSFGGGSGAGAGGGGMFGSGGGGGGTGSTGPGYSFPH

SEQ ID NO: 163 - protein, 86 aa, synthetic construct
IDDENGSVILQDDDYDDGNNHIPFEDDDVYNYNDNDDDDERIEFEDDDDDDDDSIDNDSVMDRKQPHKAEDESEDVEDVERVSKKD

SEQ ID NO: 164 - protein, 41 aa, synthetic construct
PESMREEYRKEGSKRIKCPDCEPFCNKRGSPESMREEYRKE

SEQ ID NO: 165 - protein, 68 aa, synthetic construct
RSYSPTSPNYSPTSPSGSYSPTSPNYSPTSPSGGSRSYSPTSPNYSPTSPSGSYSPTSPNYSPTSPSG

SEQ ID NO: 166 - protein, 59 aa, unidentified
  source 1..59: note = Influenza A virus
PESMREEYRKEGSSLLTEVETPGSPESMREEYRKEGSSLLTEVETPGSPESMREEYRKE

SEQ ID NO: 167 - protein, 51 aa, synthetic construct
  source 1..51: note = Influenza A virus
LIEEVRHRLKTTENSGSLIEEVRHRLKTTENSGSLIEEVRHRLKTTENSGS

SEQ ID NO: 168 - protein, 37 aa, synthetic construct
FPPEVEEQDDGTLPMSCAQESGMDRHPAACASARINV

SEQ ID NO: 169 - protein, 40 aa, Mus musculus
SHGFPPEVEEQAAGTLPMSCAQESGMDRHPAACASARINV

SEQ ID NO: 170 - DNA, 65 bp, synthetic construct
  source 1..65: mol_type = other DNA
taggcgtgtacggtgggaggcctatataagcagagctcgtttagtgaaccgtcagatcgcctgga

SEQ ID NO: 171 - DNA, 25 bp, synthetic construct
  source 1..25: mol_type = other DNA
tctagagggtatataatgggggcca

SEQ ID NO: 172 - DNA, 63 bp, synthetic construct
  source 1..63: mol_type = other DNA
ttcgcatattaaggtgacgcgtgtggcctcgaacaccgagcgaccctgcagcgacccgcttaa

SEQ ID NO: 173 - DNA, 659 bp, synthetic construct
  source 1..659: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 174 - DNA, 251 bp, synthetic construct
  source 1..251: mol_type = other DNA
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 175 - protein, 60 aa, synthetic construct
FNVLMVHKRSHTGERPLQCEICGFTCRQKGNLLRHIKLHTGEKPFKCHLCNYACQRRDAL

SEQ ID NO: 176 - protein, 4 aa, synthetic construct
PRAE

SEQ ID NO: 177 - skipped

SEQ ID NO: 178 - protein, 9 aa, synthetic construct
  VARIANT 5: note = X can be A, Y, P, S, or F
  VARIANT 6: note = X can be V, L, S, I, Y, T, or A
PRAEXXKGG

SEQ ID NO: 179 - protein, 9 aa, synthetic construct
PRAEAVKGG

SEQ ID NO: 180 - protein, 9 aa, synthetic construct
PRAEALKGG

SEQ ID NO: 181 - protein, 9 aa, synthetic construct
PRAEYSKGG

SEQ ID NO: 182 - protein, 9 aa, synthetic construct
PRAEPIKGG

SEQ ID NO: 183 - protein, 9 aa, synthetic construct
PRAEAYKGG

SEQ ID NO: 184 - protein, 9 aa, synthetic construct
PRAESSKGG

SEQ ID NO: 185 - protein, 9 aa, synthetic construct
PRAEFTKGG

SEQ ID NO: 186 - protein, 9 aa, synthetic construct
PRAEAAKGG

SEQ ID NO: 187 - protein, 9 aa, synthetic construct
DEPHYSQRR

SEQ ID NO: 188 - protein, 10 aa, synthetic construct
PPLGPIFNPG

SEQ ID NO: 189 - protein, 9 aa, synthetic construct
PLAQAYRSS

SEQ ID NO: 190 - protein, 10 aa, synthetic construct
TPIDSSFNPD

SEQ ID NO: 191 - protein, 10 aa, synthetic construct
VTPEPIFSLI

SEQ ID NO: 192 - protein, 21 aa, synthetic construct
PFFFCCFIAVAMGIRFIIMVA

SEQ ID NO: 193 - DNA, 63 bp, synthetic construct
  source 1..63: mol_type = other DNA
cccttcttcttctgttgctttatcgccgtggccatgggcatccgcttcatcattatggtggcc

SEQ ID NO: 194-197 - skipped (4 entries)

SEQ ID NO: 198 - protein, 14 aa, synthetic construct
ITQGLAVSTISSFF

SEQ ID NO: 199 - protein, 135 aa, synthetic construct
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 200 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 201 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 202 - protein, 217 aa, Homo sapiens
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIR

SEQ ID NO: 203 - protein, 541 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 204 - protein, 46 aa, synthetic construct
LLPSWAITLISVNGIFVICCLTYCFAPRCRERRRNERLRRESVRPV

SEQ ID NO: 205 - protein, 21 aa, Homo sapiens
IYIWAPLAGTCGVLLLSLVIT

SEQ ID NO: 206 - protein, 27 aa, Homo sapiens
IYIWAPLAGTCGVLLLSLVITLYCNHR

SEQ ID NO: 207 - protein, 28 aa, Homo sapiens
IYIWAPLAGTCGVLLLSLVITLYCNHRN

SEQ ID NO: 208 - protein, 12 aa, synthetic construct
GGCKJSGGCKJS

SEQ ID NO: 209 - protein, 86 aa, synthetic construct
AAAFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 210 - protein, 190 aa, synthetic construct
AATMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDEPHYSQRRLLPSWAITLISVNGIFVICCLTYCFAPRCRERRRNERLRRESVRPV

SEQ ID NO: 211 - protein, 191 aa, synthetic construct
AATMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSVTPEPIFSLILLPSWAITLISVNGIFVICCLTYCFAPRCRERRRNERLRRESVRPV

SEQ ID NO: 212 - protein, 217 aa, synthetic construct
AATMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSDEPHYSQRRSGGGGSGGGGSGGGGSGGGGSGGGSLQLLPSWAITLISVNGIFVICCLTYCFAPRCRERRRNERLRRESVRPV

SEQ ID NO: 213 - protein, 218 aa, synthetic construct
AATMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSVTPEPIFSLISGGGGSGGGGSGGGGSGGGGSGGGSLQLLPSWAITLISVNGIFVICCLTYCFAPRCRERRRNERLRRESVRPV

SEQ ID NO: 214 - protein, 217 aa, synthetic construct
AATMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGDEPHYSQRRGGGSGGGGSGGGSLQLLPSWAITLISVNGIFVICCLTYCFAPRCRERRRNERLRRESVRPV

SEQ ID NO: 215 - protein, 218 aa, synthetic construct
AATMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGVTPEPIFSLIGGGSGGGGSGGGSLQLLPSWAITLISVNGIFVICCLTYCFAPRCRERRRNERLRRESVRPV

SEQ ID NO: 216 - protein, 217 aa, synthetic construct
AATMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGGGGSGGGGSGGGSLQDEPHYSQRRLLPSWAITLISVNGIFVICCLTYCFAPRCRERRRNERLRRESVRPV

SEQ ID NO: 217 - protein, 218 aa, synthetic construct
AATMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGGGGSGGGGSGGGSLQVTPEPIFSLILLPSWAITLISVNGIFVICCLTYCFAPRCRERRRNERLRRESVRPV

SEQ ID NO: 218 - DNA, 140 bp, synthetic construct
  source 1..140: mol_type = other DNA
gggactttccactggggactttccactggggactttccactggggactttccactggggactttccactcctgcaggagctggcgcgccagacgctagcggggggctataaaagggggtgggggcgttcgtcctcactct

SEQ ID NO: 219 - protein, 44 aa, synthetic construct
GSGQCTNYALLKLAGDVESNPGPGSGEGRGSLLTCGDVEENPGP

SEQ ID NO: 220 - DNA, 132 bp, synthetic construct
  source 1..132: mol_type = other DNA
ggtagcggccagtgtaccaactacgccctgctgaaactggccggcgacgtggaatctaatcctggacctggatctggcgagggacgcgggagtctactgacgtgtggagacgtggaggaaaaccctggacct

SEQ ID NO: 221 - protein, 41 aa, synthetic construct
QCTNYALLKLAGDVESNPGPGSGEGRGSLLTCGDVEENPGP

SEQ ID NO: 222 - DNA, 123 bp, synthetic construct
  source 1..123: mol_type = other DNA
cagtgtaccaactacgccctgctgaaactggccggcgacgtggaatctaatcctggacctggatctggcgagggacgcgggagtctactgacgtgtggagacgtggaggaaaaccctggacct

SEQ ID NO: 223 - DNA, 123 bp, synthetic construct
  source 1..123: mol_type = other DNA
cagtgcacaaattatgcactgctgaagctcgccggggatgtcgagagtaacccaggacctggaagcggagaaggtcgtggtagtctactaacgtgtggtgatgtagaagaaaatcctggacct

SEQ ID NO: 224 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 225 - DNA, 342 bp, Homo sapiens
  source 1..342: mol_type = other DNA
aattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagagaagaacatcaaagagtttctgcagagcttcgtccacatcgtgcagatgttcatcaacacctca

SEQ ID NO: 226 - protein, 215 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGVTPEPIFSLIGGGSGGGGSGGGSLQLLPSWAITLISVNGIFVICCLTYCFAPRCRERRRNERLRRESVRPV

SEQ ID NO: 227 - DNA, 645 bp, synthetic construct
  source 1..645: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagagaagaacatcaaagagtttctgcagagcttcgtccacatcgtgcagatgttcatcaacacctcatcaggcggcggtggtagtggaggcggaggctcaggcgtgacccctgagcctatcttcagcctgatcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtg

SEQ ID NO: 228 - protein, 18 aa, Homo sapiens
MDWTWILFLVAAATRVHS

SEQ ID NO: 229 - DNA, 54 bp, Homo sapiens
  source 1..54: mol_type = other DNA
atggactggacttggatactctttctggtcgctgccgccacacgggtgcactct

SEQ ID NO: 230 - protein, 12 aa, synthetic construct
SGGGGSGGGGSG

SEQ ID NO: 231 - protein, 15 aa, synthetic construct
GGGSGGGGSGGGSLQ

SEQ ID NO: 232 - skipped

SEQ ID NO: 233 - protein, 6 aa, synthetic construct
GGSGGS

SEQ ID NO: 234 - protein, 9 aa, synthetic construct
GGSGGSGGS

SEQ ID NO: 235 - protein, 12 aa, synthetic construct
GGSGGSGGSGGS

SEQ ID NO: 236 - protein, 15 aa, synthetic construct
GGSGGSGGSGGSGGS

SEQ ID NO: 237 - protein, 4 aa, synthetic construct
GGGS

SEQ ID NO: 238 - protein, 8 aa, synthetic construct
GGGSGGGS

SEQ ID NO: 239 - protein, 12 aa, synthetic construct
GGGSGGGSGGGS

SEQ ID NO: 240 - protein, 16 aa, synthetic construct
GGGSGGGSGGGSGGGS

SEQ ID NO: 241 - protein, 20 aa, synthetic construct
GGGSGGGSGGGSGGGSGGGS

SEQ ID NO: 242 - protein, 5 aa, synthetic construct
GGGGS

SEQ ID NO: 243 - protein, 10 aa, synthetic construct
GGGGSGGGGS

SEQ ID NO: 244 - protein, 15 aa, synthetic construct
GGGGSGGGGSGGGGS

SEQ ID NO: 245 - protein, 20 aa, synthetic construct
GGGGSGGGGSGGGGSGGGGS

SEQ ID NO: 246 - protein, 25 aa, synthetic construct
GGGGSGGGGSGGGGSGGGGSGGGGS

SEQ ID NO: 247 - protein, 18 aa, synthetic construct
GSTSGSGKPGSGEGSTKG

SEQ ID NO: 248 - protein, 20 aa, synthetic construct
EAAAKEAAAKEAAAKEAAAK

SEQ ID NO: 249 - DNA, 54 bp, synthetic construct
  source 1..54: mol_type = other DNA
ggctctacatctggctctggcaaacctggaagcggcgagggatctaccaagggc

SEQ ID NO: 250 - protein, 37 aa, synthetic construct
SGGGGSGGGGSGVTPEPIFSLIGGGSGGGGSGGGSLQ

SEQ ID NO: 251 - DNA, 111 bp, synthetic construct
  source 1..111: mol_type = other DNA
tcaggcggcggtggtagtggaggcggaggctcaggcgtgacccctgagcctatcttcagcctgatcggcggaggttccggaggtggcggttccggcggaggatctcttcaa

SEQ ID NO: 252 - DNA, 138 bp, synthetic construct
  source 1..138: mol_type = other DNA
ttgctgcctagctgggccatcacactgatctccgtgaacggcatcttcgtgatctgctgcctgacctactgcttcgcccctagatgcagagagcggagaagaaacgagcggctgagaagagaaagcgtgcggcctgtg

SEQ ID NO: 253 - DNA, 45 bp, synthetic construct
  source 1..45: mol_type = other DNA
ggaggcggaggatctggtggcggaggaagtggcggaggcggttct

SEQ ID NO: 254 - DNA, 15 bp, synthetic construct
  source 1..15: mol_type = other DNA
ggcggcggtggctct

SEQ ID NO: 255 - DNA, 15 bp, synthetic construct
  source 1..15: mol_type = other DNA
ggtggcggcggatcc

SEQ ID NO: 256 - DNA, 30 bp, synthetic construct
  source 1..30: mol_type = other DNA
ggaggcggaggatctggtggtggtggatct

SEQ ID NO: 257 - DNA, 30 bp, synthetic construct
  source 1..30: mol_type = other DNA
ggtggcggaggaagtggcggcggaggctct

SEQ ID NO: 258 - DNA, 30 bp, synthetic construct
  source 1..30: mol_type = other DNA
ggcggtggcggatctggcggaggtggcagt

SEQ ID NO: 259 - protein, 21 aa, synthetic construct
VIGILVAVILLLLLLLLLFLI

SEQ ID NO: 260 - DNA, 63 bp, synthetic construct
  source 1..63: mol_type = other DNA
gtgatcggcattctggtcgccgtgatcctgctcctgttgctcctgctgcttctgttcctgatc

SEQ ID NO: 261 - DNA, 81 bp, synthetic construct
  source 1..81: mol_type = other DNA
atctatatctgggcccctctggctggcacatgcggagttctgctgctcagcctggtcatcaccctgtactgcaaccacaga

SEQ ID NO: 262 - protein, 42 aa, synthetic construct
AAAIEVMYPPPYLDNEKSNGTIIHVKGKHLCPSPLFPGPSKP

SEQ ID NO: 263 - protein, 12 aa, synthetic construct
ESKYGPPCPSCP

SEQ ID NO: 264 - protein, 12 aa, synthetic construct
ESKYGPPAPSAP

SEQ ID NO: 265 - protein, 12 aa, synthetic construct
ESKYGPPCPPCP

SEQ ID NO: 266 - protein, 12 aa, synthetic construct
EPKSCDKTHTCP

SEQ ID NO: 267 - protein, 86 aa, synthetic construct; identical to SEQ ID NO: 209

SEQ ID NO: 268 - protein, 132 aa, synthetic construct
ACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEADAEC

SEQ ID NO: 269 - protein, 34 aa, synthetic construct
ACPTGLYTHSGECCKACNLGEGVAQPCGANQTVC

SEQ ID NO: 270 - protein, 20 aa, synthetic construct
AVGQDTQEVIVVPHSLPFKV

SEQ ID NO: 271 - protein, 45 aa, synthetic construct
TTTPAPRPPTPAPTIALQPLSLRPEACRPAAGGAVHTRGLDFACD

SEQ ID NO: 272 - protein, 66 aa, synthetic construct
ALSNSIMYFSHFVPVFLPAKPTTTPAPRPPTPAPTIASQPLSLRPEACRPAAGGAVHTRGLDFACD

SEQ ID NO: 273 - protein, 55 aa, synthetic construct
FVPVFLPAKPTTTPAPRPPTPAPTIALQPLSLRPEACRPAAGGAVHTRGLDFACD

SEQ ID NO: 274 - DNA, 126 bp, synthetic construct
  source 1..126: mol_type = other DNA
gcagcagctatcgaggtgatgtatcctccgccctacctggataatgaaaagagtaatgggactatcattcatgtaaaagggaagcatctttgtccttctccccttttccccggtccgtctaaacct

SEQ ID NO: 275 - DNA, 36 bp, synthetic construct
  source 1..36: mol_type = other DNA
gaaagcaagtacggtccaccttgccctagctgtccg

SEQ ID NO: 276 - DNA, 36 bp, synthetic construct
  source 1..36: mol_type = other DNA
gaatccaagtacggccccccagcgcctagtgcccca

SEQ ID NO: 277 - DNA, 36 bp, synthetic construct
  source 1..36: mol_type = other DNA
gaatctaaatatggcccgccatgcccgccttgccca

SEQ ID NO: 278 - DNA, 36 bp, synthetic construct
  source 1..36: mol_type = other DNA
gaaccgaagtcttgtgataaaactcatacgtgcccg

SEQ ID NO: 279 - DNA, 258 bp, synthetic construct
  source 1..258: mol_type = other DNA
gctgctgctttcgtacccgtgttcctccctgctaagcctacgactacccccgcaccgagaccacccacgccagcacccacgattgctagccagccccttagtttgcgaccagaagcttgtcggcctgctgctggtggcgcggtacatacccgcggccttgattttgcttgcgatatatatatctgggcgcctctggccggaacatgcggggtcctcctcctttctctggttattactctctactgtaatcacaggaat

SEQ ID NO: 280 - DNA, 396 bp, synthetic construct
  source 1..396: mol_type = other DNA
gcctgcccgaccgggctctacactcatagcggggaatgttgtaaggcatgtaacttgggtgagggcgtcgcacagccctgcggagctaaccaaacagtgtgcgaaccctgcctcgatagtgtgacgttctctgatgttgtatcagctacagagccttgcaaaccatgtactgagtgcgttggacttcagtcaatgagcgctccatgtgtggaggcagatgatgcggtctgtcgatgtgcttacggatactaccaagacgagacaacagggcggtgcgaggcctgtagagtttgtgaggcgggctccgggctggtgttttcatgtcaagacaagcaaaatacggtctgtgaagagtgccctgatggcacctactcagacgaagcagatgcagaatgc

SEQ ID NO: 281 - DNA, 102 bp, synthetic construct
  source 1..102: mol_type = other DNA
gcctgccctacaggactctacacgcatagcggtgagtgttgtaaagcatgcaacctcggggaaggtgtagcccagccatgcggggctaaccaaaccgtttgc

SEQ ID NO: 282 - DNA, 60 bp, synthetic construct
  source 1..60: mol_type = other DNA
gctgtgggccaggacacgcaggaggtcatcgtggtgccacactccttgccctttaaggtg

SEQ ID NO: 283 - DNA, 135 bp, synthetic construct
  source 1..135: mol_type = other DNA
acaacaacacccgcacctcggcctccaactccagctccaacaattgcactgcaacccctgagtctgaggcccgaggcctgtaggccagcagctggcggagctgttcacactagaggcctggactttgcctgtgac

SEQ ID NO: 284 - DNA, 198 bp, synthetic construct
  source 1..198: mol_type = other DNA
gccctgagcaacagcatcatgtacttcagccacttcgtgcccgtgtttctgcccgccaagcctacaacaacccctgctcctagaccacctacaccagctcctacaatcgccagccagcctctgtctctgaggcccgaagcttgtagaccagctgctggcggagccgtgcatacaagaggactggattttgcctgcgac

SEQ ID NO: 285 - protein, 168 aa, synthetic construct
LRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 286 - DNA, 504 bp, synthetic construct
  source 1..504: mol_type = other DNA
ctgcggcacagaaggcagggcaagcactggacaagcacccagagaaaggccgactttcagcatcctgctggcgccgttggacctgagcctacagatagaggactgcagtggcggtctagccctgccgctgatgcccaagaggaaaatctttacgccgccgtgaagcacacccagcctgaggatggcgtggaaatggacaccagatctccccacgatgaggaccctcaggccgtgacatacgcagaagtgaagcactccagacctcggagagagatggcaagccctccatctcctctgagcggcgagttcctggacaccaaagacagacaggccgaagaggacagacagatggataccgaagccgccgcttctgaagccccacaggatgtgacatatgcccagctgcatagcctgacactgcggagagaagccacagagcctccaccttctcaagaaggcccatctcctgccgtgccttccatctatgccactctggccattcac

SEQ ID NO: 287 - protein, 41 aa, synthetic construct
RSKRSRLLHSDYMNMTPRRPGPTRKHYQPYAPPRDFAAYRS

SEQ ID NO: 288 - DNA, 123 bp, synthetic construct
  source 1..123: mol_type = other DNA
agaagcaagcggagcagactgctgcacagcgactacatgaacatgacccctagacggcccggacctaccagaaagcactaccagccttacgctcctcctagagatttcgccgcctaccggtcc

SEQ ID NO: 289 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 290 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 291 - protein, 5 aa, synthetic construct
RYDMH

SEQ ID NO: 292 - protein, 7 aa, synthetic construct
GFTFSRY

SEQ ID NO: 293 - protein, 7 aa, synthetic construct
GFTLSRY

SEQ ID NO: 294 - protein, 7 aa, synthetic construct
GFSFSRY

SEQ ID NO: 295 - protein, 7 aa, synthetic construct
GFSLSRY

SEQ ID NO: 296 - protein, 16 aa, synthetic construct
VIWGNGNTHYHSALKS

SEQ ID NO: 297 - protein, 5 aa, synthetic construct
WGNGN

SEQ ID NO: 298 - protein, 4 aa, synthetic construct
RIKD

SEQ ID NO: 299 - protein, 16 aa, synthetic construct
KSSQSLVASDENTYLN

SEQ ID NO: 300 - protein, 7 aa, synthetic construct
QVSKLDS

SEQ ID NO: 301 - protein, 9 aa, synthetic construct
LQGIHLPWT

SEQ ID NO: 302 - protein, 112 aa, synthetic construct
EVQLVESGGGLVQPGGSLRLSCAASGFTFSRYDMHWVRQAPGKGLEWVSVIWGNGNTHYHSALKSRFTISRDNSKNTLYLQMNSLRAEDTAVYYCTLRIKDWGQGTMVTVSS

SEQ ID NO: 303 - protein, 112 aa, synthetic construct
EVQLVESGGGLVQPGGSLRLSCAASGFTLSRYDMHWVRQAPGKGLEWVSVIWGNGNTHYHSALKSRFTISRDNSKNTLYLQMNSLRAEDTAVYYCTLRIKDWGQGTMVTVSS

SEQ ID NO: 304 - protein, 112 aa, synthetic construct
EVQLVESGGGLVQPGGSLRLSCAASGFSFSRYDMHWVRQAPGKGLEWVSVIWGNGNTHYHSALKSRFTISRDNSKNTLYLQMNSLRAEDTAVYYCTLRIKDWGQGTMVTVSS

SEQ ID NO: 305 - protein, 112 aa, synthetic construct
EVQLVESGGGLVQPGGSLRLSCAASGFSLSRYDMHWVRQAPGKGLEWVSVIWGNGNTHYHSALKSRFTISRDNSKNTLYLQMNSLRAEDTAVYYCTLRIKDWGQGTMVTVSS

SEQ ID NO: 306 - protein, 112 aa, synthetic construct
QVQLVESGGGVVQPGRSLRLSCAASGFTFSRYDMHWVRQAPGKGLEWVAVIWGNGNTHYHSALKSRFTISRDNSKNTLYLQMNSLRAEDTAVYYCTLRIKDWGQGTMVTVSS

SEQ ID NO: 307 - protein, 112 aa, synthetic construct
QVQLVESGGGVVQPGRSLRLSCAASGFTLSRYDMHWVRQAPGKGLEWVAVIWGNGNTHYHSALKSRFTISRDNSKNTLYLQMNSLRAEDTAVYYCTLRIKDWGQGTMVTVSS

SEQ ID NO: 308 - protein, 112 aa, synthetic construct
QVQLVESGGGVVQPGRSLRLSCAASGFSFSRYDMHWVRQAPGKGLEWVAVIWGNGNTHYHSALKSRFTISRDNSKNTLYLQMNSLRAEDTAVYYCTLRIKDWGQGTMVTVSS

SEQ ID NO: 309 - protein, 112 aa, synthetic construct
QVQLVESGGGVVQPGRSLRLSCAASGFSLSRYDMHWVRQAPGKGLEWVAVIWGNGNTHYHSALKSRFTISRDNSKNTLYLQMNSLRAEDTAVYYCTLRIKDWGQGTMVTVSS

SEQ ID NO: 310 - protein, 112 aa, synthetic construct
DVVMTQSPLSLPVTLGQPASISCKSSQSLVASDENTYLNWFQQRPGQSPRRLIYQVSKLDSGVPDRFSGSGSGTDFTLKISRVEAEDVGVYYCLQGIHLPWTFGQGTKLEIK

SEQ ID NO: 311 - protein, 239 aa, synthetic construct
EVQLVESGGGLVQPGGSLRLSCAASGFTFSRYDMHWVRQAPGKGLEWVSVIWGNGNTHYHSALKSRFTISRDNSKNTLYLQMNSLRAEDTAVYYCTLRIKDWGQGTMVTVSSGGGGSGGGGSGGGGSDVVMTQSPLSLPVTLGQPASISCKSSQSLVASDENTYLNWFQQRPGQSPRRLIYQVSKLDSGVPDRFSGSGSGTDFTLKISRVEAEDVGVYYCLQGIHLPWTFGQGTKLEIK

SEQ ID NO: 312 - protein, 717 aa, synthetic construct
GAGGTGCAGCTGGTTGAATCTGGCGGAGGACTGGTTCAGCCTGGCGGATCTCTGAGACTGTCTTGTGCCGCCAGCGGCTTCACCTTCAGCAGATACGATATGCACTGGGTCCGACAGGCCCCTGGCAAAGGACTTGAATGGGTGTCCGTGATCTGGGGCAACGGCAACACACACTACCACAGCGCCCTGAAGTCCCGGTTCACCATCTCCAGAGACAACAGCAAGAACACCCTGTACCTGCAGATGAACAGCCTGAGAGCCGAGGACACCGCCGTGTACTACTGCACCCTGAGAATCAAGGATTGGGGCCAGGGCACCATGGTCACCGTTTCTTCTGGAGGCGGAGGATCTGGTGGCGGAGGAAGTGGCGGAGGCGGTTCTGACGTGGTCATGACACAGAGCCCTCTGAGCCTGCCTGTGACACTGGGACAGCCTGCCAGCATCAGCTGCAAGTCTAGCCAGTCTCTGGTGGCCAGCGACGAGAACACCTACCTGAACTGGTTCCAGCAGAGGCCCGGACAGTCTCCTAGACGGCTGATCTACCAGGTGTCCAAGCTGGATAGCGGCGTGCCCGATAGATTTTCTGGCAGCGGCTCTGGCACCGACTTCACCCTGAAGATCAGCAGAGTGGAAGCCGAGGACGTGGGCGTGTACTACTGTCTGCAAGGCATCCATCTGCCTTGGACCTTTGGCCAGGGCACAAAGCTGGAAATCAAG

SEQ ID NO: 313 - protein, 118 aa, synthetic construct
EVQLVQSGAEVKKPGASVKVSCKASGYTFTSYYMHWARQAPGQGLEWMGIINPSGGSTSYAQKFQGRVTMTRDTSTSTVYMELSSLRSEDTAVYYCARVVAAAVADYWGQGTLVTVSS

SEQ ID NO: 314 - protein, 112 aa, synthetic construct
DVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQSLQTPFTFGPGTKVDIK

SEQ ID NO: 315 - protein, 122 aa, synthetic construct
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSS

SEQ ID NO: 316 - protein, 107 aa, synthetic construct
DIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIK

SEQ ID NO: 317 - protein, 119 aa, synthetic construct
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGVGAHDAFDIWGQGTTVTVSS

SEQ ID NO: 318 - protein, 112 aa, synthetic construct
DVVMTQSPLSLPVTPGEPASISCRSSQSLLHSNGNNYLDWYLQKPGQSPQLLIYLGSNRASGVPDRFSGSGSDTDFTLQISRVEAEDVGVYYCMQGTHPAISFGQGTRLEIK

SEQ ID NO: 319 - protein, 118 aa, synthetic construct
QVQLQQPGAELVKPGASLKLSCKSSGYTFTSYWMHWVRQRPGHGLEWIGEIDPSDSYKDYNQKFKDKATLTVDRSSNTAYMHLSSLTSDDSAVYYCARAITTTPFDFWGQGTTLTVSS

SEQ ID NO: 320 - protein, 108 aa, synthetic construct
DIVLTQSPATLSVTPGDSVSLSCRASQSISNNLHWYQQKSHESPRLLIKYASQSISGIPSRFSGSGSGTDFTLSINSVETEDFGVYFCQQSNTWPYTFGGGTKLEIKR

SEQ ID NO: 321 - protein, 124 aa, synthetic construct
QVTLKESGPTLVKPTETLTLTCTLSGFSLNNARMGVSWIRQPPGKCLEWLAHIFSNDEKSYSTSLKNRLTISKDSSKTQVVLTMTNVDPVDTATYYCARIVGYGSGWYGFFDYWGQGTLVTVSS

SEQ ID NO: 322 - protein, 107 aa, synthetic construct
DIQMTQSPSSLSASVGDRVTITCRASQGIRNDLGWYQQKPGKAPKRLIYAASTLQSGVPSRFSGSGSGTEFTLTISSLQPEDFATYYCLQHNSYPLTFGCGTKVEIK

SEQ ID NO: 323 - protein, 124 aa, synthetic construct
QVTLKESGPVLVKPTETLTLTCTVSGFSLRNARMAVSWIRQPPGKTLEWLAHIFSNDEKSYSTSLKSRLTISKDTSKSQVVLTMTNMDPVDTATYYCARIVGYGSGWYGYFDYWGQGTLVTVSS

SEQ ID NO: 324 - protein, 107 aa, synthetic construct
DIQMTQSPSSVSASVGDRVTITCRASQDIRYDLAWYQQKPGKAPKRLIYAASSLQSGVPSRFSGSGSGTEFTLTISSLQPEDFATYYCLQHNFYPLTFGGGTKVEIK

SEQ ID NO: 325 - protein, 116 aa, synthetic construct
EVQLVESGGGLVQPGGSLRLSCAASGFTFSSYGMHWVRQAPGKGLEWVAVISYDGSNKYYADSVKGRFTISRDNSKNTLYLQMNSLRAEDTAVYYCANLAPWAAYWGQGTLVTVSS

SEQ ID NO: 326 - protein, 112 aa, synthetic construct
EIVLTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPHTFGQGTKLEIK

SEQ ID NO: 327 - protein, 123 aa, synthetic construct
QVQLKQSGPGLVQPSQSLSITCTVSGFSLTNYGLHWVRQSPGKGLEWLGVIWSGGSTDYNAAFISRLSISKDNSKSQVFFKMNSLQADDTAIYYCARKGGIYYANHYYAMDYWGQGTSVTVSS

SEQ ID NO: 328 - protein, 114 aa, synthetic construct
DIVMTQSPSSLSVSAGEKVTMSCKSSQSLLNSGNQKNYMAWYQQKPGQPPKLLIYGASTRESGVPDRFTGSGSGTDFTLTISSVQAEDLAVYYCQNDHSYPLTFGAGTKLELKR

SEQ ID NO: 329 - protein, 116 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 330 - protein, 111 aa, synthetic construct
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 331 - protein, 116 aa, synthetic construct [sequence omitted]

SEQ ID NO: 332 - protein, 112 aa, synthetic construct
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKR

SEQ ID NO: 333 - DNA, 354 bp, synthetic construct
  source 1..354: mol_type = other DNA
gaagtgcaacttgttcagagcggggcagaagttaagaagccaggcgcttccgtcaaggtgagttgcaaggcaagtggatacacctttacgagttattatatgcactgggcacggcaggcccctggtcagggcctcgaatggatggggattataaatccttctggcgggtcaaccagctacgcacaaaaatttcaaggtcgggtgacaatgacgcgcgacacgtcaacgagtacagtgtatatggaattgtctagcctgaggtccgaggatactgctgtctattattgtgctcgcgtggtcgctgctgctgtggcagactactggggtcagggtacacttgtgacggtaagcagc

SEQ ID NO: 334 - DNA, 336 bp, synthetic construct
  source 1..336: mol_type = other DNA
gacgtagttatgacacagtctccactgtcattgccagtaacaccaggtgagcccgcctccatctcatgtagatcctcccaatctctccttcattcaaacgggtataattatctcgactggtatttgcagaaaccgggccagagccctcaactgctcatctatttggggagcaaccgggcctctggtgtccctgatagattctccgggagtggatcaggtacggattttacactgaagatcagcagggtggaagcagaagatgttggtgtgtattactgtatgcaatcactccagaccccgtttacctttgggcctggaacaaaggtagatattaaa

SEQ ID NO: 335 - DNA, 366 bp, synthetic construct
  source 1..366: mol_type = other DNA
gaggttcaactggtacaaagcggagccgaggtaaagaaaccagggagtagcgtcaaagtgtcctgcaaagcctcaggcggcacattcagtagctatgctatttcatgggtacgccaagcaccaggacaggggctggagtggatgggcgggattatccccatcttcggtacggcaaactatgcacaaaagttccagggacgagtcaccatcacggctgataagtccacctccaccgcctatatggagctgagttcccttcggagcgaggatactgctgtgtattattgtgccacgttcgcactgttcggttttcgggagcaggcgtttgatatttggggacaaggcacaacggtcacggtcagttca

SEQ ID NO: 336 - DNA, 321 bp, synthetic construct
  source 1..321: mol_type = other DNA
gacattcagatgacccagagtccctcttcattgagtgcgagcgtcggtgatcgggttacgataacctgtagggcctcccaaagtatatcatcatatttgaactggtaccaacagaaacctgggaaagcgccgaagctccttatctatgctgccagctctttgcaaagcggtgtgccctcacggttctccggtagtgggtccgggaccgacttcactttgaccatcagcagccttcagccagaggatcttgccacttattactgccagcaatcttatagcacaccgtttacattcggtccaggcacaaaggtagacattaag

SEQ ID NO: 337 - DNA, 357 bp, synthetic construct
  source 1..357: mol_type = other DNA
gaggtacagcttgtgcagagtggagcagaagttaaaaaacccggagcttccgtgaaggtaagctgcaaggcttcaggatatacatttactagctactacatgcactgggtccgccaagctccgggccaaggccttgaatggatgggcatcataaatcccagtggaggctcaacgagctatgcacaaaagttccaagggcgcgttaccatgacgcgcgacaccagcacgtccaccgtctatatggaactctcaagtttgcgatctgaagatacggctgtctactattgcgcacgaggggtcggagcgcatgacgccttcgacatctggggacaagggactacagtaactgtgtcaagc

SEQ ID NO: 338 - DNA, 147 bp, synthetic construct
  source 1..147: mol_type = other DNA
gatgttgttatgacacagtctcccctctctttgcctgttacgcctggcgagcccgcctctatttcttgtcgatctagtcagagcctgctgcattctaatggaaacaactatttggactggtacttgcaaaagccgggtcaaagtccc

SEQ ID NO: 339 - DNA, 354 bp, synthetic construct
  source 1..354: mol_type = other DNA
caagtccaacttcagcagccaggcgctgagttggttaaaccgggcgcaagcctcaaacttagttgcaagtcatccggatatactttcacgtcttattggatgcattgggtacgacaaagacctggtcacggcctcgaatggattggcgaaatcgacccgtcagacagctacaaggattacaaccagaaattcaaagataaggcaacacttactgtggatcgctcaagtaacacggcttacatgcacctctcttcactcacgtctgacgacagtgcggtgtattattgcgcccgcgctattacaacaacccctttcgatttctggggccagggtactacgctcacagtctcatcc

SEQ ID NO: 340 - DNA, 324 bp, synthetic construct
  source 1..324: mol_type = other DNA
gatatcgtcctcacccaatccccggctactttgagtgtaacaccaggcgacagcgtgtcactgtcatgccgagcctcccagtcaatcagcaataatctgcattggtatcaacagaaatcacacgaatccccccgacttttgataaagtatgcgtcacagtccatatcaggcattcccagtaggttttcaggcagtggttcaggtactgacttcaccctctccattaactctgtagaaacagaggactttggcgtctacttctgtcagcaatccaacacctggccttatacattcggcggcggcactaagctggaaattaagaga

SEQ ID NO: 341 - DNA, 372 bp, synthetic construct
  source 1..372: mol_type = other DNA
caagtaacccttaaagagtccggccccactttggttaaacctactgaaacacttacactcacatgcacattgtccggcttttcactcaacaacgcaaggatgggtgtgtcctggattcgccagccccctggaaaatgtttggaatggctcgctcatatatttagcaacgacgagaaaagttactcaacttcactcaagaaccgcctcactattagcaaagattcctccaaaacccaagtagttctgacaatgacgaatgtagacccagtcgatactgcaacttactattgcgcacgaatagtcggttacgggagtggctggtatgggtttttcgactattggggacagggcactcttgtaacagtaagtagc

SEQ ID NO: 342 - DNA, 321 bp, synthetic construct
  source 1..321: mol_type = other DNA
gacatccagatgactcaatctccatctagcctctcagcgtctgtgggcgatcgagtcaccatcacctgtagggcttcccagggtataaggaatgatttgggctggtaccagcaaaaaccgggtaaggctccgaaacgactgatatacgcagcttctacgttgcaatccggggtgccatccagatttagtggcagcgggagcggtactgagtttacgctgactatctcctcacttcagccagaggatttcgccacgtactattgtctgcaacacaactcctatccgctgaccttcgggtgcgggacaaaggtggaaattaaa

SEQ ID NO: 343 - DNA, 372 bp, synthetic construct
  source 1..372: mol_type = other DNA
caagtgaccttgaaggagtcagggccagtgttggtaaaacctactgagactctcacgttgacatgcacggtatcaggtttcagcctgaggaacgctcggatggccgtcagttggatacgccagccgccaggcaaaactcttgaatggttggcgcacatattcagtaacgacgagaaatcttactctacatcccttaagtctcgcctcaccatttctaaagacacatccaaatcacaagtggtactcacgatgacaaacatggaccctgttgacactgctacatattattgtgctaggatagtgggctacggtagcggatggtacggttattttgattactggggacaagggacgcttgttacggtgtcctca

SEQ ID NO: 344 - skipped

SEQ ID NO: 345 - DNA, 321 bp, synthetic construct
  source 1..321: mol_type = other DNA
gacattcagatgacccagtctccgtccagcgttagcgcaagcgtgggggatagagtcactattacgtgtagagccagtcaagatatacggtacgatcttgcttggtatcagcaaaaaccgggaaaagccccgaagagacttatatatgcagcttcctccttgcaaagcggggtcccatcccggtttagtggtagtggttccggaacagagttcacgctgactatttcatcactgcaacccgaagattttgccacctactactgccttcaacacaatttctatcctcttaccttcggcggaggtactaaggtagagattaag

SEQ ID NO: 346 - DNA, 354 bp, synthetic construct
  source 1..354: mol_type = other DNA
gaagtacagttggttgagagtggtggaggactcgttcaacctggcggtagtttgcgactcagctgcgcggcttccggtttcaccttctcatcctatgggatgcactgggtcagacaagcccctggaaagggcctcgaatgggttgctgtgattagctatgacggctctaataaatactatgcagatagtgtaaaggggagatttacgatttctcgcgataatagcaaaaatacgctgtacctgcaaatggaaaccaacagcctgcgagcggaagatacggcggtttattactgcgcgaatcttgccccgtgggcagcatactggggacaggggacgttggtgacggtaagcagt

SEQ ID NO: 347 - DNA, 348 bp, synthetic construct
  source 1..348: mol_type = other DNA
gagattgtgctcacccagtctccactcagccttcctgtaacgcccggtgagcctgcctctatatcatgccgaagttcccaaagccttctgcactcaaacggctataactacttggactggtacctccagaagcccggccaaagtcctcaactgttgatatacctggggtccaaccgggcatcaggagtacctgatagattctcaggaagtgggtcaggaaccgacttcacgctgaaaattagtcgcgtagaggcggaagatgtaggtgtgtattactgtatgcaggcgttgcaaacaccgcacacttttggacagggaaccaaactggaaataaagaccagtagtggt

SEQ ID NO: 348 - DNA, 369 bp, synthetic construct
  source 1..369: mol_type = other DNA
caggtccaactgaaacaaagcggtcccggtcttgtccagccctcccaatctctcagtattacttgcactgtgtcaggtttcagcctcacgaactacggtctgcattgggtccgccagtctccaggaaaaggcctggagtggctcggtgttatctggagtggtggaagtacggattacaatgctgcctttatctctcggctcagtatctccaaagataactctaagtcccaagtctttttcaaaatgaactctttgcaggcagatgatacggccatatactattgcgcacgcaagggtgggatctactatgcaaaccactattacgcgatggactactggggccaaggcacgagtgttaccgtgtcaagc

SEQ ID NO: 349 - DNA, 342 bp, synthetic construct
  source 1..342: mol_type = other DNA
gacatagtgatgactcagtctccgtcctctctttccgtgagtgcgggcgaaaaggttaccatgtcctgcaaaagttcacagtcacttctcaattctggcaaccaaaaaaattacatggcatggtatcaacagaaaccaggtcagccgccaaagctcctcatatatggtgcatcaacgcgagagtcaggcgtacctgacaggtttaccggatctggcagcggtacagactttactcttaccatatcaagtgtgcaggcagaggacttggcggtatactattgtcaaaacgatcatagttaccctcttacatttggcgcgggcactaaactggagctgaaacgc

SEQ ID NO: 350 - DNA, 348 bp, synthetic construct
  source 1..348: mol_type = other DNA
caggttcagctggttcagtctggcgccgaagtgaagaaacctggcagcagcgtgaaggtgtcctgcaaggccagcggctacacctttaccgactacaacatgcactgggtccgacaggcccctggacaaggacttgagtggatcggctacatctacccctacaatggcggcaccggctacaaccagaagttcaagagcaaggccaccatcaccgccgacgagagcacaaacaccgcctacatggaactgagcagcctgagaagcgaggacaccgccgtgtactactgtgccagaggcagacccgccatggattattggggacagggcaccctggtcaccgtttctagc

SEQ ID NO: 351 - DNA, 333 bp, synthetic construct
  source 1..333: mol_type = other DNA
gatatccagatgacacagagccccagcagcctgtctgccagcgtgggagatagagtgaccatcacctgtagagccagcgagagcgtggacaactacggcatcagcttcatgaactggttccagcagaagcccggcaaggcccctaagctgctgatctacgccgccagcaatcaaggcagcggagtgcctagcagattttccggctctggcagcggcaccgatttcaccctgacaatctctagcctccagcctgacgacttcgccacctactactgccagcagagcaaagaggtgccctggacattcggccagggcacaaaggtggaaatcaag

SEQ ID NO: 352 - DNA, 348 bp, synthetic construct
  source 1..348: mol_type = other DNA
gaagtgcagctggttcagtctggcgccgaagtgaagaaacctggcagcagcgtgaaggtgtcctgcaaggccagcggctacaccatcaccgacagcaacatccactgggtccgacaggctccaggccagtctcttgagtggatcggctacatctacccctacaacggcggcaccgactacaaccagaagttcaagaaccgggccacactgaccgtggacaaccctaccaataccgcctacatggaactgagcagcctgcggagcgaggacaccgccttttactactgcgtgaacggcaacccctggctggcctattggggacagggaacactggtcacagtgtctagc

SEQ ID NO: 353 - DNA, 336 bp, synthetic construct
  source 1..336: mol_type = other DNA
gatattcagctgacacagagccccagcacactgtctgcctctgtgggcgacagagtgaccatcacctgtagagccagcgagagcctggacaactacggcatcagatttctgacctggttccagcagaagcccggcaaggctcctaagctgctgatgtacgccgccagcaatcaaggcagcggagtgcctagcagattttccggctctggcagcggcacagagttcaccctgacaatctctagcctccagcctgacgacttcgccacctactactgccagcagaccaaagaggtgccctggtcctttggacagggcaccaaggtggaagtgaagcgg

SEQ ID NO: 354 - protein, 10 aa, synthetic construct
GYTFTSYYMH

SEQ ID NO: 355 - protein, 17 aa, synthetic construct
IINPSGGSTSYAQKFQG

SEQ ID NO: 356 - protein, 9 aa, synthetic construct
VVAAAVADY

SEQ ID NO: 357 - protein, 16 aa, synthetic construct
RSSQSLLHSNGYNYLD

SEQ ID NO: 358 - protein, 6 aa, synthetic construct
LGSNRA

SEQ ID NO: 359 - protein, 9 aa, synthetic construct
MQSLQTPFT

SEQ ID NO: 360 - protein, 10 aa, synthetic construct
GGTFSSYAIS

SEQ ID NO: 361 - protein, 17 aa, synthetic construct
GIIPIFGTANYAQKFQG

SEQ ID NO: 362 - protein, 13 aa, synthetic construct
FALFGFREQAFDI

SEQ ID NO: 363 - protein, 11 aa, synthetic construct
RASQSISSYLN

SEQ ID NO: 364 - protein, 7 aa, synthetic construct
AASSLQS

SEQ ID NO: 365 - protein, 9 aa, synthetic construct
QQSYSTPFT

SEQ ID NO: 366 - protein, 10 aa, synthetic construct
GYTFTSYYMH

SEQ ID NO: 367 - protein, 17 aa, synthetic construct
IINPSGGSTSYAQKFQG

SEQ ID NO: 368 - protein, 10 aa, synthetic construct
GVGAHDAFDI

SEQ ID NO: 369 - protein, 16 aa, synthetic construct
RSSQSLLHSNGNNYLD

SEQ ID NO: 370 - protein, 6 aa, synthetic construct
LGSNRA

SEQ ID NO: 371 - protein, 9 aa, synthetic construct
MQGTHPAIS

SEQ ID NO: 372 - protein, 5 aa, synthetic construct
SYWMH

SEQ ID NO: 373 - protein, 17 aa, synthetic construct
EIDPSDSYKDYNQKFKD

SEQ ID NO: 374 - protein, 9 aa, synthetic construct
AITTTPFDF

SEQ ID NO: 375 - protein, 11 aa, synthetic construct
RASQSISNNLH

SEQ ID NO: 376 - protein, 7 aa, synthetic construct
YASQSIS

SEQ ID NO: 377 - protein, 9 aa, synthetic construct
QQSNTWPYT

SEQ ID NO: 378 - protein, 7 aa, synthetic construct
NARMGVS

SEQ ID NO: 379 - protein, 16 aa, synthetic construct
HIFSNDEKSYSTSLKN

SEQ ID NO: 380 - protein, 14 aa, synthetic construct
IVGYGSGWYGFFDY

SEQ ID NO: 381 - protein, 11 aa, synthetic construct
RASQGIRNDLG

SEQ ID NO: 382 - protein, 7 aa, synthetic construct
AASTLQS

SEQ ID NO: 383 - protein, 9 aa, synthetic construct
LQHNSYPLT

SEQ ID NO: 384 - protein, 7 aa, synthetic construct
NARMAVS

SEQ ID NO: 385 - protein, 16 aa, synthetic construct
HIFSNDEKSYSTSLKS

SEQ ID NO: 386 - protein, 14 aa, synthetic construct
IVGYGSGWYGYFDY

SEQ ID NO: 387 - protein, 11 aa, synthetic construct
RASQDIRYDLA

SEQ ID NO: 388 - protein, 7 aa, synthetic construct
AASSLQS

SEQ ID NO: 389 - protein, 9 aa, synthetic construct
LQHNFYPLT

SEQ ID NO: 390 - protein, 8 aa, synthetic construct
GFTFSSYG

SEQ ID NO: 391 - protein, 8 aa, synthetic construct
ISYDGSNK

SEQ ID NO: 392 - protein, 10 aa, synthetic construct
ANLAPWAAYW

SEQ ID NO: 393 - protein, 11 aa, synthetic construct
QSLLHSNGYNY

SEQ ID NO: 394 - skipped

SEQ ID NO: 395 - protein, 9 aa, synthetic construct
MQALQTPHT

SEQ ID NO: 396 - protein, 5 aa, synthetic construct
NYGLH

SEQ ID NO: 397 - protein, 16 aa, synthetic construct
VIWSGGSTDYNAAFIS

SEQ ID NO: 398 - protein, 15 aa, synthetic construct
KGGIYYANHYYAMDY

SEQ ID NO: 399 - protein, 17 aa, synthetic construct
KSSQSLLNSGNQKNYMA

SEQ ID NO: 400 - protein, 7 aa, synthetic construct
GASTRES

SEQ ID NO: 401 - protein, 9 aa, synthetic construct
QNDHSYPLT

SEQ ID NO: 402 - protein, 5 aa, synthetic construct
DYNMH

SEQ ID NO: 403 - protein, 19 aa, synthetic construct
YIYPYNGGTGYNQKFKSKA

SEQ ID NO: 404 - protein, 10 aa, synthetic construct
GRPAMDYWGQ

SEQ ID NO: 405 - protein, 15 aa, synthetic construct
RASESVDNYGISFMN

SEQ ID NO: 406 - protein, 7 aa, synthetic construct
AASNQGS

SEQ ID NO: 407 - protein, 9 aa, synthetic construct
QQSKEVPWT

SEQ ID NO: 408 - protein, 8 aa, synthetic construct
GYTITDSN

SEQ ID NO: 409 - protein, 8 aa, synthetic construct
IYPYNGGT

SEQ ID NO: 410 - protein, 9 aa, synthetic construct
VNGNPWLAY

SEQ ID NO: 411 - protein, 10 aa, synthetic construct
ESLDNYGIRF

SEQ ID NO: 412 - skipped

SEQ ID NO: 413 - protein, 9 aa, synthetic construct
QQTKEVPWS

SEQ ID NO: 414 - protein, 752 aa, synthetic construct
MLLLVTSLLLCELPHPAFLLIPEVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGSTSGSGKPGSGEGSTKGDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIKALSNSIMYFSHFVPVFLPAKPTTTPAPRPPTPAPTIASQPLSLRPEACRPAAGGAVHTRGLDFACDIYIWAPLAGTCGVLLLSLVITLYCNHRRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 415 - protein, 754 aa, synthetic construct
MLLLVTSLLLCELPHPAFLLIPEVQLVQSGAEVKKPGSSVKVSCKASGGTFSSYAISWVRQAPGQGLEWMGGIIPIFGTANYAQKFQGRVTITADKSTSTAYMELSSLRSEDTAVYYCATFALFGFREQAFDIWGQGTTVTVSSGGGGSGGGGSQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKGGGGSGGGGSDIQMTQSPSSLSASVGDRVTITCRASQSISSYLNWYQQKPGKAPKLLIYAASSLQSGVPSRFSGSGSGTDFTLTISSLQPEDLATYYCQQSYSTPFTFGPGTKVDIKALSNSIMYFSHFVPVFLPAKPTTTPAPRPPTPAPTIASQPLSLRPEACRPAAGGAVHTRGLDFACDIYIWAPLAGTCGVLLLSLVITLYCNHRRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 416 - protein, 63 aa, synthetic construct
ATGGCTCTGCCCGTGACAGCTTTGCTGCTGCCTTTGGCACTGCTGCTGCATGCTGCTAGACCA

SEQ ID NO: 417 - protein, 52 aa, synthetic construct
HPSDPLELVVSGPSGGPSSPTTGPTSTSGPEDQPLTPTGSDPQSGLGRHLGV

SEQ ID NO: 418 - DNA, 156 bp, synthetic construct
  source 1..156: mol_type = other DNA
cacccatccgatcctctcgagctggtggtttctggaccttctggcggccctagcagccctacaacaggacctacaagcacaagcggccctgaggaccaacctctgacaccaacaggcagcgatcctcagtctggactggggagacatctgggcgtt

SEQ ID NO: 419 - protein, 505 aa, synthetic construct
MALPVTALLLPLALLLHAARPEVQLVESGGGLVQPGGSLRLSCAASGFTFSRYDMHWVRQAPGKGLEWVSVIWGNGNTHYHSALKSRFTISRDNSKNTLYLQMNSLRAEDTAVYYCTLRIKDWGQGTMVTVSSGGGGSGGGGSGGGGSDVVMTQSPLSLPVTLGQPASISCKSSQSLVASDENTYLNWFQQRPGQSPRRLIYQVSKLDSGVPDRFSGSGSGTDFTLKISRVEAEDVGVYYCLQGIHLPWTFGQGTKLEIKNGAAHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 420 - DNA, 1515 bp, synthetic construct
  source 1..1515: mol_type = other DNA
atggctctgcccgtgacagctttgctgctgcctttggcactgctgctgcatgctgctagaccagaggtgcagctggttgaatctggcggaggactggttcagcctggcggatctctgagactgtcttgtgccgccagcggcttcaccttcagcagatacgatatgcactgggtccgacaggcccctggcaaaggacttgaatgggtgtccgtgatctggggcaacggcaacacacactaccacagcgccctgaagtcccggttcaccatctccagagacaacagcaagaacaccctgtacctgcagatgaacagcctgagagccgaggacaccgccgtgtactactgcaccctgagaatcaaggattggggccagggcaccatggtcaccgtttcttctggaggcggaggatctggtggcggaggaagtggcggaggcggttctgacgtggtcatgacacagagccctctgagcctgcctgtgacactgggacagcctgccagcatcagctgcaagtctagccagtctctggtggccagcgacgagaacacctacctgaactggttccagcagaggcccggacagtctcctagacggctgatctaccaggtgtccaagctggatagcggcgtgcccgatagattttctggcagcggctctggcaccgacttcaccctgaagatcagcagagtggaagccgaggacgtgggcgtgtactactgtctgcaaggcatccatctgccttggacctttggccagggcacaaagctggaaatcaagaatggcgctgcacacccatccgatcctctcgagctggtggtttctggaccttctggcggccctagcagccctacaacaggacctacaagcacaagcggccctgaggaccaacctctgacaccaacaggcagcgatcctcagtctggactggggagacatctgggcgttgtgatcggcattctggtcgccgtgatcctgctcctgttgctcctgctgcttctgttcctgatcctgcggcacagaaggcagggcaagcactggacaagcacccagagaaaggccgactttcagcatcctgctggcgccgttggacctgagcctacagatagaggactgcagtggcggtctagccctgccgctgatgcccaagaggaaaatctttacgccgccgtgaagcacacccagcctgaggatggcgtggaaatggacaccagatctccccacgatgaggaccctcaggccgtgacatacgcagaagtgaagcactccagacctcggagagagatggcaagccctccatctcctctgagcggcgagttcctggacaccaaagacagacaggccgaagaggacagacagatggataccgaagccgccgcttctgaagccccacaggatgtgacatatgcccagctgcatagcctgacactgcggagagaagccacagagcctccaccttctcaagaaggcccatctcctgccgtgccttccatctatgccactctggccattcac

SEQ ID NO: 421 - protein, 498 aa, synthetic construct
MALPVTALLLPLALLLHAARPEVQLVESGGGLVQPGGSLRLSCAASGFTFSRYDMHWVRQAPGKGLEWVSVIWGNGNTHYHSALKSRFTISRDNSKNTLYLQMNSLRAEDTAVYYCTLRIKDWGQGTMVTVSSGGGGSGGGGSGGGGSDVVMTQSPLSLPVTLGQPASISCKSSQSLVASDENTYLNWFQQRPGQSPRRLIYQVSKLDSGVPDRFSGSGSGTDFTLKISRVEAEDVGVYYCLQGIHLPWTFGQGTKLEIKNGAATTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 422 - DNA, 1494 bp, synthetic construct
  source 1..1494: mol_type = other DNA
atggctctgcccgtgacagctttgctgctgcctttggcactgctgctgcatgctgctagaccagaggtgcagctggttgaatctggcggaggactggttcagcctggcggatctctgagactgtcttgtgccgccagcggcttcaccttcagcagatacgatatgcactgggtccgacaggcccctggcaaaggacttgaatgggtgtccgtgatctggggcaacggcaacacacactaccacagcgccctgaagtcccggttcaccatctccagagacaacagcaagaacaccctgtacctgcagatgaacagcctgagagccgaggacaccgccgtgtactactgcaccctgagaatcaaggattggggccagggcaccatggtcaccgtttcttctggaggcggaggatctggtggcggaggaagtggcggaggcggttctgacgtggtcatgacacagagccctctgagcctgcctgtgacactgggacagcctgccagcatcagctgcaagtctagccagtctctggtggccagcgacgagaacacctacctgaactggttccagcagaggcccggacagtctcctagacggctgatctaccaggtgtccaagctggatagcggcgtgcccgatagattttctggcagcggctctggcaccgacttcaccctgaagatcagcagagtggaagccgaggacgtgggcgtgtactactgtctgcaaggcatccatctgccttggacctttggccagggcacaaagctggaaatcaagaatggcgctgcaacaacaacacccgcacctcggcctccaactccagctccaacaattgcactgcaacccctgagtctgaggcccgaggcctgtaggccagcagctggcggagctgttcacactagaggcctggactttgcctgtgacgtgatcggcattctggtcgccgtgatcctgctcctgttgctcctgctgcttctgttcctgatcctgcggcacagaaggcagggcaagcactggacaagcacccagagaaaggccgactttcagcatcctgctggcgccgttggacctgagcctacagatagaggactgcagtggcggtctagccctgccgctgatgcccaagaggaaaatctttacgccgccgtgaagcacacccagcctgaggatggcgtggaaatggacaccagatctccccacgatgaggaccctcaggccgtgacatacgcagaagtgaagcactccagacctcggagagagatggcaagccctccatctcctctgagcggcgagttcctggacaccaaagacagacaggccgaagaggacagacagatggataccgaagccgccgcttctgaagccccacaggatgtgacatatgcccagctgcatagcctgacactgcggagagaagccacagagcctccaccttctcaagaaggcccatctcctgccgtgccttccatctatgccactctggccattcac

SEQ ID NO: 423 - protein, 22 aa, synthetic construct
MLLLVTSLLLCELPHPAFLLIP

SEQ ID NO: 424 - DNA, 66 bp, synthetic construct
  source 1..66: mol_type = other DNA
atgctgctgctggttacatctctgctgctgtgcgagctgccccatcctgcctttctgcttattcct

SEQ ID NO: 425 - DNA, 11857 bp, synthetic construct
  source 1..11857: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctggatccgcgtcaagtggagcaaggcaggtggacagtctcgaggccgccaccatggactggacttggatactctttctggtcgctgccgccacacgggtgcactctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagagaagaacatcaaagagtttctgcagagcttcgtccacatcgtgcagatgttcatcaacacctcatcaggcggcggtggtagtggaggcggaggctcaggcgtgacccctgagcctatcttcagcctgatcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtgggtagcggccagtgtaccaactacgccctgctgaaactggccggcgacgtggaatctaatcctggacctggatctggcgagggacgcgggagtctactgacgtgtggagacgtggaggaaaaccctggacctatgctgctgctggttacatctctgctgctgtgcgagctgccccatcctgcctttctgcttattcctgaagttcagctggttcagagcggagccgaagtgaagaaacctggcagcagcgtgaaggtgtcctgcaaagcttctggcggcaccttcagcagctacgccatctcttgggttcgacaggcacctggacagggcctcgagtggatgggaggaatcatccctatcttcggcaccgccaattacgcccagaaatttcagggaagagtgaccatcaccgccgacaagagcaccagcaccgcctacatggaactgagcagcctgagaagcgaggataccgctgtgtattactgcgccacattcgccctgttcggcttcagagagcaggccttcgatatctggggccagggcacaaccgtgacagtttcatctggcggcggtggctctcaggttcagcttgtgcaatctggcgctgaagtgaaaaagcccggctcctccgtgaaagtgtcttgtaaagccagcggctacacctttaccgactacaatatgcattgggtccgccaggcaccaggccagggacttgaatggatcggctacatctacccctacaacggcggcaccggctacaaccagaagttcaagtccaaggccacaatcacagccgacgagtccaccaatactgcttatatggaactgtctagccttcggagcgaggacacagccgtgtactattgcgctagaggcagacccgccatggactattggggacagggaaccctggtcacagtgtccagcggctctacatctggctctggcaaacctggaagcggcgagggatctaccaagggcgacatccagatgacacagagccctagtagcctgagcgccagcgtgggagacagagtgacaattacctgtcgggccagcgagagcgtggacaactacggcatcagcttcatgaactggttccagcagaagcctggcaaggcccctaagctgctgatctatgccgccagcaatcaaggcagcggagtgccctcaagattcagcggaagcggctccggcaccgactttaccctgacaatcagctccctgcagcctgacgacttcgccacctactactgccagcagagcaaagaggtgccctggacattcggacagggcaccaaggtggaaatcaaaggtggcggcggatccgatattcagatgactcagtccccaagcagcctgtcagcctccgtgggcgatagagtcaccattacatgcagagccagccagtccatctccagctacctgaactggtatcagcaaaaacccggaaaggctccaaagctcctcatctacgccgcttctagcctgcagtctggcgtgccatctagattctctggctccggaagcgggaccgacttcacactgaccatatcttctctgcagcccgaagatctggccacgtactattgtcagcagtcctacagcacccctttcaccttcggacccggaacaaaggtggacattaaggccctgagcaacagcatcatgtacttcagccacttcgtgcccgtgtttctgcccgccaagcctacaacaacccctgctcctagaccacctacaccagctcctacaatcgccagccagcctctgtctctgaggcccgaagcttgtagaccagctgctggcggagccgtgcatacaagaggactggattttgcctgcgacatctatatctgggcccctctggctggcacatgcggagttctgctgctcagcctggtcatcaccctgtactgcaaccacagaagaagcaagcggagcagactgctgcacagcgactacatgaacatgacccctagacggcccggacctaccagaaagcactaccagccttacgctcctcctagagatttcgccgcctaccggtccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggttctggccagtgcacaaattatgcactgctgaagctcgccggggatgtcgagagtaacccaggacctggaagcggagaaggtcgtggtagtctactaacgtgtggtgatgtagaagaaaatcctggacctatggctctgcccgtgacagctttgctgctgcctttggcactgctgctgcatgctgctagaccagaggtgcagctggttgaatctggcggaggactggttcagcctggcggatctctgagactgtcttgtgccgccagcggcttcaccttcagcagatacgatatgcactgggtccgacaggcccctggcaaaggacttgaatgggtgtccgtgatctggggcaacggcaacacacactaccacagcgccctgaagtcccggttcaccatctccagagacaacagcaagaacaccctgtacctgcagatgaacagcctgagagccgaggacaccgccgtgtactactgcaccctgagaatcaaggattggggccagggcaccatggtcaccgtttcttctggaggcggaggatctggtggcggaggaagtggcggaggcggttctgacgtggtcatgacacagagccctctgagcctgcctgtgacactgggacagcctgccagcatcagctgcaagtctagccagtctctggtggccagcgacgagaacacctacctgaactggttccagcagaggcccggacagtctcctagacggctgatctaccaggtgtccaagctggatagcggcgtgcccgatagattttctggcagcggctctggcaccgacttcaccctgaagatcagcagagtggaagccgaggacgtgggcgtgtactactgtctgcaaggcatccatctgccttggacctttggccagggcacaaagctggaaatcaagaatggcgctgcaacaacaacacccgcacctcggcctccaactccagctccaacaattgcactgcaacccctgagtctgaggcccgaggcctgtaggccagcagctggcggagctgttcacactagaggcctggactttgcctgtgacgtgatcggcattctggtcgccgtgatcctgctcctgttgctcctgctgcttctgttcctgatcctgcggcacagaaggcagggcaagcactggacaagcacccagagaaaggccgactttcagcatcctgctggcgccgttggacctgagcctacagatagaggactgcagtggcggtctagccctgccgctgatgcccaagaggaaaatctttacgccgccgtgaagcacacccagcctgaggatggcgtggaaatggacaccagatctccccacgatgaggaccctcaggccgtgacatacgcagaagtgaagcactccagacctcggagagagatggcaagccctccatctcctctgagcggcgagttcctggacaccaaagacagacaggccgaagaggacagacagatggataccgaagccgccgcttctgaagccccacaggatgtgacatatgcccagctgcatagcctgacactgcggagagaagccacagagcctccaccttctcaagaaggcccatctcctgccgtgccttccatctatgccactctggccattcactaatgaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 426 - DNA, 11878 bp, synthetic construct
  source 1..11878: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctggatccgcgtcaagtggagcaaggcaggtggacagtctcgaggccgccaccatggactggacttggatactctttctggtcgctgccgccacacgggtgcactctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagagaagaacatcaaagagtttctgcagagcttcgtccacatcgtgcagatgttcatcaacacctcatcaggcggcggtggtagtggaggcggaggctcaggcgtgacccctgagcctatcttcagcctgatcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtgggtagcggccagtgtaccaactacgccctgctgaaactggccggcgacgtggaatctaatcctggacctggatctggcgagggacgcgggagtctactgacgtgtggagacgtggaggaaaaccctggacctatgctgctgctggttacatctctgctgctgtgcgagctgccccatcctgcctttctgcttattcctgaagttcagctggttcagagcggagccgaagtgaagaaacctggcagcagcgtgaaggtgtcctgcaaagcttctggcggcaccttcagcagctacgccatctcttgggttcgacaggcacctggacagggcctcgagtggatgggaggaatcatccctatcttcggcaccgccaattacgcccagaaatttcagggaagagtgaccatcaccgccgacaagagcaccagcaccgcctacatggaactgagcagcctgagaagcgaggataccgctgtgtattactgcgccacattcgccctgttcggcttcagagagcaggccttcgatatctggggccagggcacaaccgtgacagtttcatctggcggcggtggctctcaggttcagcttgtgcaatctggcgctgaagtgaaaaagcccggctcctccgtgaaagtgtcttgtaaagccagcggctacacctttaccgactacaatatgcattgggtccgccaggcaccaggccagggacttgaatggatcggctacatctacccctacaacggcggcaccggctacaaccagaagttcaagtccaaggccacaatcacagccgacgagtccaccaatactgcttatatggaactgtctagccttcggagcgaggacacagccgtgtactattgcgctagaggcagacccgccatggactattggggacagggaaccctggtcacagtgtccagcggctctacatctggctctggcaaacctggaagcggcgagggatctaccaagggcgacatccagatgacacagagccctagtagcctgagcgccagcgtgggagacagagtgacaattacctgtcgggccagcgagagcgtggacaactacggcatcagcttcatgaactggttccagcagaagcctggcaaggcccctaagctgctgatctatgccgccagcaatcaaggcagcggagtgccctcaagattcagcggaagcggctccggcaccgactttaccctgacaatcagctccctgcagcctgacgacttcgccacctactactgccagcagagcaaagaggtgccctggacattcggacagggcaccaaggtggaaatcaaaggtggcggcggatccgatattcagatgactcagtccccaagcagcctgtcagcctccgtgggcgatagagtcaccattacatgcagagccagccagtccatctccagctacctgaactggtatcagcaaaaacccggaaaggctccaaagctcctcatctacgccgcttctagcctgcagtctggcgtgccatctagattctctggctccggaagcgggaccgacttcacactgaccatatcttctctgcagcccgaagatctggccacgtactattgtcagcagtcctacagcacccctttcaccttcggacccggaacaaaggtggacattaaggccctgagcaacagcatcatgtacttcagccacttcgtgcccgtgtttctgcccgccaagcctacaacaacccctgctcctagaccacctacaccagctcctacaatcgccagccagcctctgtctctgaggcccgaagcttgtagaccagctgctggcggagccgtgcatacaagaggactggattttgcctgcgacatctatatctgggcccctctggctggcacatgcggagttctgctgctcagcctggtcatcaccctgtactgcaaccacagaagaagcaagcggagcagactgctgcacagcgactacatgaacatgacccctagacggcccggacctaccagaaagcactaccagccttacgctcctcctagagatttcgccgcctaccggtccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggttctggccagtgcacaaattatgcactgctgaagctcgccggggatgtcgagagtaacccaggacctggaagcggagaaggtcgtggtagtctactaacgtgtggtgatgtagaagaaaatcctggacctatggctctgcccgtgacagctttgctgctgcctttggcactgctgctgcatgctgctagaccagaggtgcagctggttgaatctggcggaggactggttcagcctggcggatctctgagactgtcttgtgccgccagcggcttcaccttcagcagatacgatatgcactgggtccgacaggcccctggcaaaggacttgaatgggtgtccgtgatctggggcaacggcaacacacactaccacagcgccctgaagtcccggttcaccatctccagagacaacagcaagaacaccctgtacctgcagatgaacagcctgagagccgaggacaccgccgtgtactactgcaccctgagaatcaaggattggggccagggcaccatggtcaccgtttcttctggaggcggaggatctggtggcggaggaagtggcggaggcggttctgacgtggtcatgacacagagccctctgagcctgcctgtgacactgggacagcctgccagcatcagctgcaagtctagccagtctctggtggccagcgacgagaacacctacctgaactggttccagcagaggcccggacagtctcctagacggctgatctaccaggtgtccaagctggatagcggcgtgcccgatagattttctggcagcggctctggcaccgacttcaccctgaagatcagcagagtggaagccgaggacgtgggcgtgtactactgtctgcaaggcatccatctgccttggacctttggccagggcacaaagctggaaatcaagaatggcgctgcacacccatccgatcctctcgagctggtggtttctggaccttctggcggccctagcagccctacaacaggacctacaagcacaagcggccctgaggaccaacctctgacaccaacaggcagcgatcctcagtctggactggggagacatctgggcgttgtgatcggcattctggtcgccgtgatcctgctcctgttgctcctgctgcttctgttcctgatcctgcggcacagaaggcagggcaagcactggacaagcacccagagaaaggccgactttcagcatcctgctggcgccgttggacctgagcctacagatagaggactgcagtggcggtctagccctgccgctgatgcccaagaggaaaatctttacgccgccgtgaagcacacccagcctgaggatggcgtggaaatggacaccagatctccccacgatgaggaccctcaggccgtgacatacgcagaagtgaagcactccagacctcggagagagatggcaagccctccatctcctctgagcggcgagttcctggacaccaaagacagacaggccgaagaggacagacagatggataccgaagccgccgcttctgaagccccacaggatgtgacatatgcccagctgcatagcctgacactgcggagagaagccacagagcctccaccttctcaagaaggcccatctcctgccgtgccttccatctatgccactctggccattcactaatgaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 427 - DNA, 11863 bp, synthetic construct
  source 1..11863: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctggatccgcgtcaagtggagcaaggcaggtggacagtctcgaggccgccaccatggactggacttggatactctttctggtcgctgccgccacacgggtgcactctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagagaagaacatcaaagagtttctgcagagcttcgtccacatcgtgcagatgttcatcaacacctcatcaggcggcggtggtagtggaggcggaggctcaggcgtgacccctgagcctatcttcagcctgatcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtgggtagcggccagtgtaccaactacgccctgctgaaactggccggcgacgtggaatctaatcctggacctggatctggcgagggacgcgggagtctactgacgtgtggagacgtggaggaaaaccctggacctatgctgctgctggttacatctctgctgctgtgcgagctgccccatcctgcctttctgcttattcctgaagtgcagctggttcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacaagagcacaagcaccgcctacatggaactgagcagcctgagaagcgaggacaccgccgtgtactactgcgccacatttgccctgttcggcttcagagagcaggccttcgatatctggggccagggcacaaccgtgaccgtttctagcggaggcggaggatctggtggtggtggatctcaggttcagctggtgcagagcggagctgaagtgaaaaagccaggctcctccgtgaaagtctcttgcaaggccagcggctacacctttaccgactacaacatgcactgggtccgacaagctccaggccagggacttgagtggatcggctacatctacccctacaacggcggcaccggctacaaccagaagttcaagagcaaggccacaatcacagccgacgagtccaccaacacagcttatatggaactgtctagcctgcgctccgaggatacagctgtgtactattgtgccaggggcagacccgccatggactattggggacagggaaccctggtcacagtgtcatctggtggcggaggaagtggcggcggaggctctgatattcagatgactcagagccccagcagcctgtctgcctctgtgggagacagagtgacaattacctgccgggccagcgagagcgtggacaattacggcatcagcttcatgaactggttccagcagaagcccggcaaggcccctaagctgctgatctacgccgccagcaatcaaggcagcggagtgcctagcagattttccggctctggcagcggcaccgatttcaccctgaccatcagtagcctgcagcctgacgacttcgccacctactactgccagcagagcaaagaggtgccctggacctttggacagggcaccaaggtggaaatcaaaggcggtggcggatctggcggaggtggcagtgatatccaaatgacccagtctccatccagcctgagcgccagcgttggcgatagagtcacaatcacctgtagagccagccagagcatcagctcctacctgaattggtatcagcagaaaccaggcaaagctcccaaactcctgatctatgctgcctccagcctgcagagtggcgtgccatctagattcagcggcagcggaagcggcacagactttacactgacaatatcttccctgcagccagaggacctggccacatattattgccagcagtcctacagcacccctttcaccttcggacccggaacaaaggtggacattaaggccctgagcaacagcatcatgtacttcagccacttcgtgcccgtgtttctgcccgccaagcctacaacaacccctgctcctagaccacctacaccagctcctacaatcgccagccagcctctgtctctgaggcccgaagcttgtagaccagctgctggcggagccgtgcatacaagaggactggattttgcctgcgacatctatatctgggcccctctggctggcacatgcggagttctgctgctcagcctggtcatcaccctgtactgcaaccacagaagaagcaagcggagcagactgctgcacagcgactacatgaacatgacccctagacggcccggacctaccagaaagcactaccagccttacgctcctcctagagatttcgccgcctaccggtccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggttctggccagtgcacaaattatgcactgctgaagctcgccggggatgtcgagagtaacccaggacctggaagcggagaaggtcgtggtagtctactaacgtgtggtgatgtagaagaaaatcctggacctatggctctgcccgtgacagctttgctgctgcctttggcactgctgctgcatgctgctagaccagaggtgcagctggttgaatctggcggaggactggttcagcctggcggatctctgagactgtcttgtgccgccagcggcttcaccttcagcagatacgatatgcactgggtccgacaggcccctggcaaaggacttgaatgggtgtccgtgatctggggcaacggcaacacacactaccacagcgccctgaagtcccggttcaccatctccagagacaacagcaagaacaccctgtacctgcagatgaacagcctgagagccgaggacaccgccgtgtactactgcaccctgagaatcaaggattggggccagggcaccatggtcaccgtttcttctggaggcggaggatctggtggcggaggaagtggcggaggcggttctgacgtggtcatgacacagagccctctgagcctgcctgtgacactgggacagcctgccagcatcagctgcaagtctagccagtctctggtggccagcgacgagaacacctacctgaactggttccagcagaggcccggacagtctcctagacggctgatctaccaggtgtccaagctggatagcggcgtgcccgatagattttctggcagcggctctggcaccgacttcaccctgaagatcagcagagtggaagccgaggacgtgggcgtgtactactgtctgcaaggcatccatctgccttggacctttggccagggcacaaagctggaaatcaagaatggcgctgcaacaacaacacccgcacctcggcctccaactccagctccaacaattgcactgcaacccctgagtctgaggcccgaggcctgtaggccagcagctggcggagctgttcacactagaggcctggactttgcctgtgacgtgatcggcattctggtcgccgtgatcctgctcctgttgctcctgctgcttctgttcctgatcctgcggcacagaaggcagggcaagcactggacaagcacccagagaaaggccgactttcagcatcctgctggcgccgttggacctgagcctacagatagaggactgcagtggcggtctagccctgccgctgatgcccaagaggaaaatctttacgccgccgtgaagcacacccagcctgaggatggcgtggaaatggacaccagatctccccacgatgaggaccctcaggccgtgacatacgcagaagtgaagcactccagacctcggagagagatggcaagccctccatctcctctgagcggcgagttcctggacaccaaagacagacaggccgaagaggacagacagatggataccgaagccgccgcttctgaagccccacaggatgtgacatatgcccagctgcatagcctgacactgcggagagaagccacagagcctccaccttctcaagaaggcccatctcctgccgtgccttccatctatgccactctggccattcactaatgaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 428 - DNA, 11884 bp, synthetic construct
  source 1..11884: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctggatccgcgtcaagtggagcaaggcaggtggacagtctcgaggccgccaccatggactggacttggatactctttctggtcgctgccgccacacgggtgcactctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagagaagaacatcaaagagtttctgcagagcttcgtccacatcgtgcagatgttcatcaacacctcatcaggcggcggtggtagtggaggcggaggctcaggcgtgacccctgagcctatcttcagcctgatcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtgggtagcggccagtgtaccaactacgccctgctgaaactggccggcgacgtggaatctaatcctggacctggatctggcgagggacgcgggagtctactgacgtgtggagacgtggaggaaaaccctggacctatgctgctgctggttacatctctgctgctgtgcgagctgccccatcctgcctttctgcttattcctgaagtgcagctggttcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacaagagcacaagcaccgcctacatggaactgagcagcctgagaagcgaggacaccgccgtgtactactgcgccacatttgccctgttcggcttcagagagcaggccttcgatatctggggccagggcacaaccgtgaccgtttctagcggaggcggaggatctggtggtggtggatctcaggttcagctggtgcagagcggagctgaagtgaaaaagccaggctcctccgtgaaagtctcttgcaaggccagcggctacacctttaccgactacaacatgcactgggtccgacaagctccaggccagggacttgagtggatcggctacatctacccctacaacggcggcaccggctacaaccagaagttcaagagcaaggccacaatcacagccgacgagtccaccaacacagcttatatggaactgtctagcctgcgctccgaggatacagctgtgtactattgtgccaggggcagacccgccatggactattggggacagggaaccctggtcacagtgtcatctggtggcggaggaagtggcggcggaggctctgatattcagatgactcagagccccagcagcctgtctgcctctgtgggagacagagtgacaattacctgccgggccagcgagagcgtggacaattacggcatcagcttcatgaactggttccagcagaagcccggcaaggcccctaagctgctgatctacgccgccagcaatcaaggcagcggagtgcctagcagattttccggctctggcagcggcaccgatttcaccctgaccatcagtagcctgcagcctgacgacttcgccacctactactgccagcagagcaaagaggtgccctggacctttggacagggcaccaaggtggaaatcaaaggcggtggcggatctggcggaggtggcagtgatatccaaatgacccagtctccatccagcctgagcgccagcgttggcgatagagtcacaatcacctgtagagccagccagagcatcagctcctacctgaattggtatcagcagaaaccaggcaaagctcccaaactcctgatctatgctgcctccagcctgcagagtggcgtgccatctagattcagcggcagcggaagcggcacagactttacactgacaatatcttccctgcagccagaggacctggccacatattattgccagcagtcctacagcacccctttcaccttcggacccggaacaaaggtggacattaaggccctgagcaacagcatcatgtacttcagccacttcgtgcccgtgtttctgcccgccaagcctacaacaacccctgctcctagaccacctacaccagctcctacaatcgccagccagcctctgtctctgaggcccgaagcttgtagaccagctgctggcggagccgtgcatacaagaggactggattttgcctgcgacatctatatctgggcccctctggctggcacatgcggagttctgctgctcagcctggtcatcaccctgtactgcaaccacagaagaagcaagcggagcagactgctgcacagcgactacatgaacatgacccctagacggcccggacctaccagaaagcactaccagccttacgctcctcctagagatttcgccgcctaccggtccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggttctggccagtgcacaaattatgcactgctgaagctcgccggggatgtcgagagtaacccaggacctggaagcggagaaggtcgtggtagtctactaacgtgtggtgatgtagaagaaaatcctggacctatggctctgcccgtgacagctttgctgctgcctttggcactgctgctgcatgctgctagaccagaggtgcagctggttgaatctggcggaggactggttcagcctggcggatctctgagactgtcttgtgccgccagcggcttcaccttcagcagatacgatatgcactgggtccgacaggcccctggcaaaggacttgaatgggtgtccgtgatctggggcaacggcaacacacactaccacagcgccctgaagtcccggttcaccatctccagagacaacagcaagaacaccctgtacctgcagatgaacagcctgagagccgaggacaccgccgtgtactactgcaccctgagaatcaaggattggggccagggcaccatggtcaccgtttcttctggaggcggaggatctggtggcggaggaagtggcggaggcggttctgacgtggtcatgacacagagccctctgagcctgcctgtgacactgggacagcctgccagcatcagctgcaagtctagccagtctctggtggccagcgacgagaacacctacctgaactggttccagcagaggcccggacagtctcctagacggctgatctaccaggtgtccaagctggatagcggcgtgcccgatagattttctggcagcggctctggcaccgacttcaccctgaagatcagcagagtggaagccgaggacgtgggcgtgtactactgtctgcaaggcatccatctgccttggacctttggccagggcacaaagctggaaatcaagaatggcgctgcacacccatccgatcctctcgagctggtggtttctggaccttctggcggccctagcagccctacaacaggacctacaagcacaagcggccctgaggaccaacctctgacaccaacaggcagcgatcctcagtctggactggggagacatctgggcgttgtgatcggcattctggtcgccgtgatcctgctcctgttgctcctgctgcttctgttcctgatcctgcggcacagaaggcagggcaagcactggacaagcacccagagaaaggccgactttcagcatcctgctggcgccgttggacctgagcctacagatagaggactgcagtggcggtctagccctgccgctgatgcccaagaggaaaatctttacgccgccgtgaagcacacccagcctgaggatggcgtggaaatggacaccagatctccccacgatgaggaccctcaggccgtgacatacgcagaagtgaagcactccagacctcggagagagatggcaagccctccatctcctctgagcggcgagttcctggacaccaaagacagacaggccgaagaggacagacagatggataccgaagccgccgcttctgaagccccacaggatgtgacatatgcccagctgcatagcctgacactgcggagagaagccacagagcctccaccttctcaagaaggcccatctcctgccgtgccttccatctatgccactctggccattcactaatgaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 429 - DNA, 11884 bp, synthetic construct
  source 1..11884: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctggatccgcgtcaagtggagcaaggcaggtggacagtctcgaggccgccaccatggactggacttggatactctttctggtcgctgccgccacacgggtgcactctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagagaagaacatcaaagagtttctgcagagcttcgtccacatcgtgcagatgttcatcaacacctcatcaggcggcggtggtagtggaggcggaggctcaggcgtgacccctgagcctatcttcagcctgatcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtgggtagcggccagtgtaccaactacgccctgctgaaactggccggcgacgtggaatctaatcctggacctggatctggcgagggacgcgggagtctactgacgtgtggagacgtggaggaaaaccctggacctatggctctgcccgtgacagctttgctgctgcctttggcactgctgctgcatgctgctagaccagaggtgcagctggttgaatctggcggaggactggttcagcctggcggatctctgagactgtcttgtgccgccagcggcttcaccttcagcagatacgatatgcactgggtccgacaggcccctggcaaaggacttgaatgggtgtccgtgatctggggcaacggcaacacacactaccacagcgccctgaagtcccggttcaccatctccagagacaacagcaagaacaccctgtacctgcagatgaacagcctgagagccgaggacaccgccgtgtactactgcaccctgagaatcaaggattggggccagggcaccatggtcaccgtttcttctggaggcggaggatctggtggcggaggaagtggcggaggcggttctgacgtggtcatgacacagagccctctgagcctgcctgtgacactgggacagcctgccagcatcagctgcaagtctagccagtctctggtggccagcgacgagaacacctacctgaactggttccagcagaggcccggacagtctcctagacggctgatctaccaggtgtccaagctggatagcggcgtgcccgatagattttctggcagcggctctggcaccgacttcaccctgaagatcagcagagtggaagccgaggacgtgggcgtgtactactgtctgcaaggcatccatctgccttggacctttggccagggcacaaagctggaaatcaagaatggcgctgcacacccatccgatcctctcgagctggtggtttctggaccttctggcggccctagcagccctacaacaggacctacaagcacaagcggccctgaggaccaacctctgacaccaacaggcagcgatcctcagtctggactggggagacatctgggcgttgtgatcggcattctggtcgccgtgatcctgctcctgttgctcctgctgcttctgttcctgatcctgcggcacagaaggcagggcaagcactggacaagcacccagagaaaggccgactttcagcatcctgctggcgccgttggacctgagcctacagatagaggactgcagtggcggtctagccctgccgctgatgcccaagaggaaaatctttacgccgccgtgaagcacacccagcctgaggatggcgtggaaatggacaccagatctccccacgatgaggaccctcaggccgtgacatacgcagaagtgaagcactccagacctcggagagagatggcaagccctccatctcctctgagcggcgagttcctggacaccaaagacagacaggccgaagaggacagacagatggataccgaagccgccgcttctgaagccccacaggatgtgacatatgcccagctgcatagcctgacactgcggagagaagccacagagcctccaccttctcaagaaggcccatctcctgccgtgccttccatctatgccactctggccattcacggttctggccagtgcacaaattatgcactgctgaagctcgccggggatgtcgagagtaacccaggacctggaagcggagaaggtcgtggtagtctactaacgtgtggtgatgtagaagaaaatcctggacctatgctgctgctggttacatctctgctgctgtgcgagctgccccatcctgcctttctgcttattcctgaagtgcagctggttcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacaagagcacaagcaccgcctacatggaactgagcagcctgagaagcgaggacaccgccgtgtactactgcgccacatttgccctgttcggcttcagagagcaggccttcgatatctggggccagggcacaaccgtgaccgtttctagcggaggcggaggatctggtggtggtggatctcaggttcagctggtgcagagcggagctgaagtgaaaaagccaggctcctccgtgaaagtctcttgcaaggccagcggctacacctttaccgactacaacatgcactgggtccgacaagctccaggccagggacttgagtggatcggctacatctacccctacaacggcggcaccggctacaaccagaagttcaagagcaaggccacaatcacagccgacgagtccaccaacacagcttatatggaactgtctagcctgcgctccgaggatacagctgtgtactattgtgccaggggcagacccgccatggactattggggacagggaaccctggtcacagtgtcatctggtggcggaggaagtggcggcggaggctctgatattcagatgactcagagccccagcagcctgtctgcctctgtgggagacagagtgacaattacctgccgggccagcgagagcgtggacaattacggcatcagcttcatgaactggttccagcagaagcccggcaaggcccctaagctgctgatctacgccgccagcaatcaaggcagcggagtgcctagcagattttccggctctggcagcggcaccgatttcaccctgaccatcagtagcctgcagcctgacgacttcgccacctactactgccagcagagcaaagaggtgccctggacctttggacagggcaccaaggtggaaatcaaaggcggtggcggatctggcggaggtggcagtgatatccaaatgacccagtctccatccagcctgagcgccagcgttggcgatagagtcacaatcacctgtagagccagccagagcatcagctcctacctgaattggtatcagcagaaaccaggcaaagctcccaaactcctgatctatgctgcctccagcctgcagagtggcgtgccatctagattcagcggcagcggaagcggcacagactttacactgacaatatcttccctgcagccagaggacctggccacatattattgccagcagtcctacagcacccctttcaccttcggacccggaacaaaggtggacattaaggccctgagcaacagcatcatgtacttcagccacttcgtgcccgtgtttctgcccgccaagcctacaacaacccctgctcctagaccacctacaccagctcctacaatcgccagccagcctctgtctctgaggcccgaagcttgtagaccagctgctggcggagccgtgcatacaagaggactggattttgcctgcgacatctatatctgggcccctctggctggcacatgcggagttctgctgctcagcctggtcatcaccctgtactgcaaccacagaagaagcaagcggagcagactgctgcacagcgactacatgaacatgacccctagacggcccggacctaccagaaagcactaccagccttacgctcctcctagagatttcgccgcctaccggtccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatgaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 430 - protein, 501 aa, synthetic construct
MALPVTALLLPLALLLHAARPEVQLVESGGGLVQPGGSLRLSCAASGFTFSRYDMHWVRQAPGKGLEWVSVIWGNGNTHYHSALKSRFTISRDNSKNTLYLQMNSLRAEDTAVYYCTLRIKDWGQGTMVTVSSGGGGSGGGGSGGGGSDVVMTQSPLSLPVTLGQPASISCKSSQSLVASDENTYLNWFQQRPGQSPRRLIYQVSKLDSGVPDRFSGSGSGTDFTLKISRVEAEDVGVYYCLQGIHLPWTFGQGTKLEIKHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 431 - protein, 494 aa, synthetic construct
MALPVTALLLPLALLLHAARPEVQLVESGGGLVQPGGSLRLSCAASGFTFSRYDMHWVRQAPGKGLEWVSVIWGNGNTHYHSALKSRFTISRDNSKNTLYLQMNSLRAEDTAVYYCTLRIKDWGQGTMVTVSSGGGGSGGGGSGGGGSDVVMTQSPLSLPVTLGQPASISCKSSQSLVASDENTYLNWFQQRPGQSPRRLIYQVSKLDSGVPDRFSGSGSGTDFTLKISRVEAEDVGVYYCLQGIHLPWTFGQGTKLEIKTTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 432 - DNA, 11833 bp, synthetic construct
  source 1..11833: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctggatccgcgtcaagtggagcaaggcaggtggacagtctcgaggccgccaccatggactggacttggatactctttctggtcgctgccgccacacgggtgcactctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagagaagaacatcaaagagtttctgcagagcttcgtccacatcgtgcagatgttcatcaacacctcatcaggcggcggtggtagtggaggcggaggctcaggcgtgacccctgagcctatcttcagcctgatcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtgggtagcggccagtgtaccaactacgccctgctgaaactggccggcgacgtggaatctaatcctggacctggatctggcgagggacgcgggagtctactgacgtgtggagacgtggaggaaaaccctggacctatggctctgcccgtgacagctttgctgctgcctttggcactgctgctgcatgctgctagaccagaggtgcagctggttgaatctggcggaggactggttcagcctggcggatctctgagactgtcttgtgccgccagcggcttcaccttcagcagatacgatatgcactgggtccgacaggcccctggcaaaggacttgaatgggtgtccgtgatctggggcaacggcaacacacactaccacagcgccctgaagtcccggttcaccatctccagagacaacagcaagaacaccctgtacctgcagatgaacagcctgagagccgaggacaccgccgtgtactactgcaccctgagaatcaaggattggggccagggcaccatggtcaccgtttcttctggaggcggaggatctggtggcggaggaagtggcggaggcggttctgacgtggtcatgacacagagccctctgagcctgcctgtgacactgggacagcctgccagcatcagctgcaagtctagccagtctctggtggccagcgacgagaacacctacctgaactggttccagcagaggcccggacagtctcctagacggctgatctaccaggtgtccaagctggatagcggcgtgcccgatagattttctggcagcggctctggcaccgacttcaccctgaagatcagcagagtggaagccgaggacgtgggcgtgtactactgtctgcaaggcatccatctgccttggacctttggccagggcacaaagctggaaatcaagaatggcgctgcacacccatccgatcctctcgagctggtggtttctggaccttctggcggccctagcagccctacaacaggacctacaagcacaagcggccctgaggaccaacctctgacaccaacaggcagcgatcctcagtctggactggggagacatctgggcgttgtgatcggcattctggtcgccgtgatcctgctcctgttgctcctgctgcttctgttcctgatcctgcggcacagaaggcagggcaagcactggacaagcacccagagaaaggccgactttcagcatcctgctggcgccgttggacctgagcctacagatagaggactgcagtggcggtctagccctgccgctgatgcccaagaggaaaatctttacgccgccgtgaagcacacccagcctgaggatggcgtggaaatggacaccagatctccccacgatgaggaccctcaggccgtgacatacgcagaagtgaagcactccagacctcggagagagatggcaagccctccatctcctctgagcggcgagttcctggacaccaaagacagacaggccgaagaggacagacagatggataccgaagccgccgcttctgaagccccacaggatgtgacatatgcccagctgcatagcctgacactgcggagagaagccacagagcctccaccttctcaagaaggcccatctcctgccgtgccttccatctatgccactctggccattcacggttctggccagtgcacaaattatgcactgctgaagctcgccggggatgtcgagagtaacccaggacctggaagcggagaaggtcgtggtagtctactaacgtgtggtgatgtagaagaaaatcctggacctatgctgctgctggttacatctctgctgctgtgcgagctgccccatcctgcctttctgcttattcctgaagtgcagctggttcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacaagagcacaagcaccgcctacatggaactgagcagcctgagaagcgaggacaccgccgtgtactactgcgccacatttgccctgttcggcttcagagagcaggccttcgatatctggggccagggcacaaccgtgaccgtttctagcggaggcggaggatctggtggtggtggatctcaggttcagctggtgcagagcggagctgaagtgaaaaagccaggctcctccgtgaaagtctcttgcaaggccagcggctacacctttaccgactacaacatgcactgggtccgacaagctccaggccagggacttgagtggatcggctacatctacccctacaacggcggcaccggctacaaccagaagttcaagagcaaggccacaatcacagccgacgagtccaccaacacagcttatatggaactgtctagcctgcgctccgaggatacagctgtgtactattgtgccaggggcagacccgccatggactattggggacagggaaccctggtcacagtgtcatctggtggcggaggaagtggcggcggaggctctgatattcagatgactcagagccccagcagcctgtctgcctctgtgggagacagagtgacaattacctgccgggccagcgagagcgtggacaattacggcatcagcttcatgaactggttccagcagaagcccggcaaggcccctaagctgctgatctacgccgccagcaatcaaggcagcggagtgcctagcagattttccggctctggcagcggcaccgatttcaccctgaccatcagtagcctgcagcctgacgacttcgccacctactactgccagcagagcaaagaggtgccctggacctttggacagggcaccaaggtggaaatcaaaggcggtggcggatctggcggaggtggcagtgatatccaaatgacccagtctccatccagcctgagcgccagcgttggcgatagagtcacaatcacctgtagagccagccagagcatcagctcctacctgaattggtatcagcagaaaccaggcaaagctcccaaactcctgatctatgctgcctccagcctgcagagtggcgtgccatctagattcagcggcagcggaagcggcacagactttacactgacaatatcttccctgcagccagaggacctggccacatattattgccagcagtcctacagcacccctttcaccttcggacccggaacaaaggtggacattaaggccctgagcaacagcatcatgtacttcagccacttcgtgcccgtgtttctgcccgccaagcctacaacaacccctgctcctagaccacctacaccagctcctacaatcgccagccagcctctgtctctgaggcccgaagcttgtagaccagctgctggcggagccgtgcatacaagaggactggattttgcctgcgacatctatatctgggcccctctggctggcacatgcggagttctgctgctcagcctggtcatcaccctgtactgcaaccacagaagaagcaagcggagcagactgctgcacagcgactacatgaacatgacccctagacggcccggacctaccagaaagcactaccagccttacgctcctcctagagatttcgccgcctaccggtccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatgaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 433 - DNA, 11795 bp, synthetic construct
  source 1..11795: mol_type = other DNA
actacaacatgcactgggtccgacaagctccaggccagggacttgagtggatcggctacatctacccctacaacggcggcaccggctacaaccagaagttcaagagcaaggccacaatcacagccgacgagtccaccaacacagcttatatggaactgtctagcctgcgctccgaggatacagctgtgtactattgtgccaggggcagacccgccatggactattggggacagggaaccctggtcacagtgtcatctggtggcggaggaagtggcggcggaggctctgatattcagatgactcagagccccagcagcctgtctgcctctgtgggagacagagtgacaattacctgccgggccagcgagagcgtggacaattacggcatcagcttcatgaactggttccagcagaagcccggcaaggcccctaagctgctgatctacgccgccagcaatcaaggcagcggagtgcctagcagattttccggctctggcagcggcaccgatttcaccctgaccatcagtagcctgcagcctgacgacttcgccacctactactgccagcagagcaaagaggtgccctggacctttggacagggcaccaaggtggaaatcaaaggcggtggcggatctggcggaggtggcagtgatatccaaatgacccagtctccatccagcctgagcgccagcgttggcgatagagtcacaatcacctgtagagccagccagagcatcagctcctacctgaattggtatcagcagaaaccaggcaaagctcccaaactcctgatctatgctgcctccagcctgcagagtggcgtgccatctagattcagcggcagcggaagcggcacagactttacactgacaatatcttccctgcagccagaggacctggccacatattattgccagcagtcctacagcacccctttcaccttcggacccggaacaaaggtggacattaaggccctgagcaacagcatcatgtacttcagccacttcgtgcccgtgtttctgcccgccaagcctacaacaacccctgctcctagaccacctacaccagctcctacaatcgccagccagcctctgtctctgaggcccgaagcttgtagaccagctgctggcggagccgtgcatacaagaggactggattttgcctgcgacatctatatctgggcccctctggctggcacatgcggagttctgctgctcagcctggtcatcaccctgtactgcaaccacagaagaagcaagcggagcagactgctgcacagcgactacatgaacatgacccctagacggcccggacctaccagaaagcactaccagccttacgctcctcctagagatttcgccgcctaccggtccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatgaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctggatccgcgtcaagtggagcaaggcaggtggacagtctcgaggccgccaccatggactggacttggatactctttctggtcgctgccgccacacgggtgcactctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagagaagaacatcaaagagtttctgcagagcttcgtccacatcgtgcagatgttcatcaacacctcatcaggcggcggtggtagtggaggcggaggctcaggcgtgacccctgagcctatcttcagcctgatcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtgggtagcggccagtgtaccaactacgccctgctgaaactggccggcgacgtggaatctaatcctggacctggatctggcgagggacgcgggagtctactgacgtgtggagacgtggaggaaaaccctggacctatggctctgcccgtgacagctttgctgctgcctttggcactgctgctgcatgctgctagaccagaggtgcagctggttgaatctggcggaggactggttcagcctggcggatctctgagactgtcttgtgccgccagcggcttcaccttcagcagatacgatatgcactgggtccgacaggcccctggcaaaggacttgaatgggtgtccgtgatctggggcaacggcaacacacactaccacagcgccctgaagtcccggttcaccatctccagagacaacagcaagaacaccctgtacctgcagatgaacagcctgagagccgaggacaccgccgtgtactactgcaccctgagaatcaaggattggggccagggcaccatggtcaccgtttcttctggaggcggaggatctggtggcggaggaagtggcggaggcggttctgacgtggtcatgacacagagccctctgagcctgcctgtgacactgggacagcctgccagcatcagctgcaagtctagccagtctctggtggccagcgacgagaacacctacctgaactggttccagcagaggcccggacagtctcctagacggctgatctaccaggtgtccaagctggatagcggcgtgcccgatagattttctggcagcggctctggcaccgacttcaccctgaagatcagcagagtggaagccgaggacgtgggcgtgtactactgtctgcaaggcatccatctgccttggacctttggccagggcacaaagctggaaatcaagaatggcgctgcacacccatccgatcctctcgagctggtggtttctggaccttctggcggccctagcagccctacaacaggacctacaagcacaagcggccctgaggaccaacctctgacaccaacaggcagcgatcctcagtctggactggggagacatctgggcgttgtgatcggcattctggtcgccgtgatcctgctcctgttgctcctgctgcttctgttcctgatcctgcggcacagaaggcagggcaagcactggacaagcacccagagaaaggccgactttcagcatcctgctggcgccgttggacctgagcctacagatagaggactgcagtggcggtctagccctgccgctgatgcccaagaggaaaatctttacgccgccgtgaagcacacccagcctgaggatggcgtggaaatggacaccagatctccccacgatgaggaccctcaggccgtgacatacgcagaagtgaagcactccagacctcggagagagatggcaagccctccatctcctctgagcggcgagttcctggacaccaaagacagacaggccgaagaggacagacagatggataccgaagccgccgcttctgaagccccacaggatgtgacatatgcccagctgcatagcctgacactgcggagagaagccacagagcctccaccttctcaagaaggcccatctcctgccgtgccttccatctatgccactctggccattcacggttctggccagtgcacaaattatgcactgctgaagctcgccggggatgtcgagagtaacccaggacctggaagcggagaaggtcgtggtagtctactaacgtgtggtgatgtagaagaaaatcctggacctatgctgctgctggttacatctctgctgctgtgcgagctgccccatcctgcctttctgcttattcctgaagtgcagctggttcagtctggcgccgaagtgaagaaacctggcagcagcgtgaaggtgtcctgcaaagcttctggcggcaccttcagcagctacgccatctcttgggttcgacaggcccctggacaaggcctggaatggatgggaggcatcatccccatcttcggcaccgccaattacgcccagaaattccagggcagagtgaccatcaccgccgacaagagcacaagcaccgcctacatggaactgagcagcctgagaagcgaggacaccgccgtgtactactgcgccacatttgccctgttcggcttcagagagcaggccttcgatatctggggccagggcacaaccgtgaccgtttctagcggaggcggaggatctggtggtggtggatctcaggttcagctggtgcagagcggagctgaagtgaaaaagccaggctcctccgtgaaagtctcttgcaaggccagcggctacacctttaccg

SEQ ID NO: 434 - skipped

SEQ ID NO: 435 - protein, 97 aa, synthetic construct
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 436 - protein, 41 aa, synthetic construct
AVSLSKMLKKRSPLTTGVYVKMPPTEPECEKQFQPYFIPIN

SEQ ID NO: 437 - protein, 111 aa, synthetic construct
RRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 438 - protein, 84 aa, synthetic construct
HLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 439 - protein, 41 aa, synthetic construct
AVSLSKMLKKRSPLTTGVGVKMPPTEPECEKQFQPYFIPIN

SEQ ID NO: 440 - protein, 70 aa, synthetic construct
KEPASPLDKCHYTKDNGQFDQSAKQLNLEAYTIEQETALISNKNGKPKRQQRKPNPPLNLDSYIVGQNDM

SEQ ID NO: 441 - protein, 82 aa, synthetic construct
LTRKKKALRIHSVEGDLRRKSAGQEEWSPSAPSPPGSCVQAEAAPAGLCGEQRGEDCAELHDYFNVLSYRSLGNCSFFTETG

SEQ ID NO: 442 - protein, 70 aa, synthetic construct
MDNQGVIYSDLNLPPNPKRQQRKPKGNKNSILATEQEITYAELNLQKASQDFQGNDKTYHCKDLPSAPEK

SEQ ID NO: 443 - protein, 217 aa, synthetic construct [sequence omitted]

SEQ ID NO: 444 - protein, 481 aa, synthetic construct [sequence omitted]

SEQ ID NO: 445 - protein, 412 aa, synthetic construct
MGDGAVKQGFLYLQQQQTFGKKWRRFGASLYGGSDCALARLELQEGPEKPRRCEAARKVIRLSDCLRVAEAGGEASSPRDTSAFFLETKERLYLLAAPAAERGDWVQAICLLAFPGQRKELSGPEGKQSRPCMEENELYSSAVTVGPHKEFAVTMRPTEASERCHLRGSYTLRAGESALELWGGPEPGTQLYDWPYRFLRRFGRDKVTFSFEAGRRCVSGEGNFEFETRQGNEIFLALEEAISAQKNAAPATPQPQPATIPASLPRPDSPYSRPHDSLPPPSPTTPVPAPRPRGQEGEYAVPFDAVARSLGKNFRGILAVPPQLLADPLYDSIEETLPPRPDHIYDEPEGVAALSLYDSPQEPRGEAWRRQATADRDPAGLQHVQPAGQDFSASGWQPGTEYDNVVLKKGPK

SEQ ID NO: 446 - protein, 268 aa, synthetic construct
PAPAERPLPNPEGLDSDFLAVLSDYPSPDISPPIFRRGEKLRVISDEGGWWKAISLSTGRESYIPGICVARVYHGWLFEGLGRDKAEELLQLPDTKVGSFMIRESETKKGFYSLSVRHRQVKHYRIFRLPNNWYYISPRLTFQCLEDLVNHYSEVADGLCCVLTTPCLTQSTAAPAVRASSSPVTLRQKTVDWRRVSRLQEDPEGTENPLGVDESLFSYGLRESIASYLSLTSEDNTSFDRKKKSISLMYGGSKRKSSFFSSPPYFED

SEQ ID NO: 447 - protein, 239 aa, synthetic construct
PAPAERPLPNPEGLDSDFLAVLSDYPSPDISPPIFRRGEKLRVISDEGGWWKAISLSTGRESYIPGICVARVYHGWLFEGLGRDKAEELLQLPDTKVGSFMIRESETKKGFYSLSVRHRQVKHYRIFRLPNNWYYISPRLTFQCLEDLVNHYSEVADGLCCVLTTPCLTQSTAAPAVRASSSPVTLRQKTVDWRRVSRLQEDPEGTENPLGVDESLFSYGLRESIASYLSLTSEDNTSF

SEQ ID NO: 448 - protein, 254 aa, synthetic construct
RKSLPSPSLSSSVQGQGPVTMEAERSKATAVALGSFPAGGPAELSLRLGEPLTIVSEDGDWWTVLSEVSGREYNIPSVHVAKVSHGWLYEGLSREKAEELLLLPGNPGGAFLIRESQTRRGSYSLSVRLSRPASWDRIRHYRIHCLDNGWLYISPRLTFPSLQALVDHYSELADDICCLLKEPCVLQRAGPLPGKDIPLPVTVQRTPLNWKELDSSLLFSEAATGEESLLSEGLRESLSFYISLNDEAVSLDDA

SEQ ID NO: 449 - protein, 61 aa, synthetic construct
KVNGCRKYKLNKTESTPVVEEDEMQPYASYTEKNNPLYDTTNKVKASEALQSEVDTDLHTL

SEQ ID NO: 450 - protein, 110 aa, synthetic construct
RIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 451 - protein, 84 aa, synthetic construct
HRWCSNKKNAAVMDQESAGNRTANSEDSDEQDPQEVTYTQLNHCVFTQRKITRPSQRPKTPPTDIIVYTELPNAESRSKVVSCP

SEQ ID NO: 452 - protein, 41 aa, synthetic construct
MTDSVIYSMLELPTATQAQNDYGPQQKSSSSRPSCSCLGSG

SEQ ID NO: 453 - protein, 101 aa, synthetic construct
HRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 454 - protein, 116 aa, synthetic construct
LRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 455 - protein, 167 aa, synthetic construct
RRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 456 - protein, 168 aa, synthetic construct
QHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 457 - protein, 141 aa, synthetic construct
KLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 458 - protein, 126 aa, synthetic construct
KILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 459 - DNA, 291 bp, synthetic construct
  source 1..291: mol_type = other DNA
tgtagcagagccgccagaggaacaatcggcgccagaagaacaggccagcctctgaaagaggacccctctgccgttcctgtgttcagcgtggactatggcgagctggatttccagtggcgggaaaagacacccgagcctccagtgccttgtgtgcctgagcagacagagtacgccaccatcgtgttccctagcggcatgggcacatctagccctgccagaagaggatctgccgacggacctagatctgcccagcctcttagacctgaggacggccactgttcttggcctctt

SEQ ID NO: 460 - DNA, 291 bp, synthetic construct
  source 1..291: mol_type = other DNA
tgtagccgagcggccagaggcacaatcggggcaagacgaacaggacagccgctcaaagaggaccccagtgcggtccccgttttctccgtggattacggagaactggatttccagtggcgggagaagacaccagagcccccggtgccctgcgtgccggagcagactgagtacgccacgattgtgtttccctctggaatggggacttcatcccccgctaggcgcggctcagctgatggcccaagatccgctcaaccgttgcggccagaggacgggcattgcagttggcctctg

SEQ ID NO: 461 - DNA, 123 bp, synthetic construct
  source 1..123: mol_type = other DNA
gccgtgtctctgagcaagatgctgaagaagcggagccctctgaccaccggcgtgtacgtgaaaatgcctcctaccgagcctgagtgcgagaagcagttccagccttacttcatccccatcaac

SEQ ID NO: 462 - DNA, 333 bp, synthetic construct
  source 1..333: mol_type = other DNA
aggagacatcaggggaagcagaatgaactcagcgatacagcagggcgagaaattaatttggtagacgcgcatctgaagtccgaacagacagaggcttctactagacagaactcccaagttttgttgagtgagacggggatctatgataatgatcccgatctgtgttttagaatgcaggagggtagtgaagtctactcaaacccgtgcctggaagaaaataagcccggcattgtttacgctagtttgaatcattctgtaataggcccgaactccagactggctcgcaatgtgaaggaggccccaactgagtatgcgtccatttgcgtgcggtct

SEQ ID NO: 463 - DNA, 333 bp, synthetic construct
  source 1..333: mol_type = other DNA
agaagacatcaggggaagcagaatgaactcagcgatacagcagggcgagaaattaatttggtagacgcgcatctgaagtccgaacagacagaggcttctactagacagaactcccaagttttgttgagtgagacggggatctatgataatgatcccgatctgtgttttagaatgcaggagggtagtgaagtctactcaaacccgtgcctggaagaaaataagcccggcattgtttacgctagtttgaatcattctgtaataggcccgaactccagactggctcgcaatgtgaaggaggccccaactgagtatgcgtccatttgcgtgcggtct

SEQ ID NO: 464 - DNA, 333 bp, synthetic construct
  source 1..333: mol_type = other DNA
agaaggcaccagggaaagcagaacgagctgagcgataccgccggcagagaaatcaacctggtggacgcccacctgaaaagcgagcagacagaggccagcaccagacagaatagccaggtgctgctgagcgagacaggcatctacgacaacgaccccgacctgtgcttccggatgcaagagggaagcgaggtgtacagcaacccctgcctggaagagaacaagcccggcatcgtgtacgctagcctgaaccactctgtgatcggccccaattccagactggcccggaacgtgaaagaggcccctacagagtacgccagcatctgcgtcagaagc

SEQ ID NO: 465 - DNA, 504 bp, synthetic construct
  source 1..504: mol_type = other DNA
ttgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcac

SEQ ID NO: 466 - DNA, 504 bp, synthetic construct
  source 1..504: mol_type = other DNA
ctgcggcacagacggcagggcaagcactggacaagcacacagagaaaggccgactttcagcaccctgctggtgccgttggacctgagcctacagatagaggactgcagtggcggtctagccctgccgctgatgctcaagaggaaaacctgtacgccgccgtgaagcacacccaacctgaagatggcgtggaaatggacaccagatctccccacgatgaggaccctcaggccgtgacatatgccgaagtgaagcactcccggcctcggagagaaatggctagccctccaagtcctctgagcggcgagttcctggacaccaaggatagacaggccgaagaggaccggcagatggatacagaagctgccgcatctgaggccccacaggatgtgacttatgcccagctgcacagcctgacactgcggagagaagccacagagcctccaccttctcaagagggcccatctccagccgtgcctagcatctatgccacactggccatccac

SEQ ID NO: 467 - DNA, 123 bp, synthetic construct
  source 1..123: mol_type = other DNA
gccgtgtcacttagtaagatgctgaagaagaggtcaccactgacgacaggggttggagtgaagatgccacccacagaacccgaatgtgagaagcaattccagccttatttcattccaataaat

SEQ ID NO: 468 - DNA, 252 bp, synthetic construct
  source 1..252: mol_type = other DNA
catctgtggtgttctaataagaagaatgctgctgtgatggatcaagagcccgctggtaacagaacggccaacagtgaagatagcgatgagcaggacccagaagaagtgacctacgcccaactcgaccactgtgtttttacgcagcggaaaatcactcgaccctctcaacgacccaaaacgccgcctacggacaccatactctacaccgaactgccgaacgccaaaccacggtccaaggtggtatcatgtccg

SEQ ID NO: 469 - DNA, 504 bp, synthetic construct
  source 1..504: mol_type = other DNA
ctgcggcacagaaggcagggcaagcactggacaagcacccagagaaaggccgattttcagcaccctgctggcgccgttggacctgagcctacagatagaggactgcagtggcggtctagccctgctgccgatgctcaagaggaaaacctgtacgccgccgtgaagcacacccaacctgaagatggcgtggaaatggacaccagatctccccacgatgaggaccctcaggccgtgacatacgctgaagtgaagcactcccggcctcggagagaaatggctagccctccaagtcctctgagcggcgagttcctggacaccaaggatagacaggccgaagaggaccggcagatggatacagaagctgccgcctctgaagccccacaggatgtgacatatgcccagctgcatagcctgacactgcggagagaagccacagagcctccaccttctcaagagggcccatctccagccgtgcctagcatctatgccacactggccattcac

SEQ ID NO: 470 - DNA, 210 bp, synthetic construct
  source 1..210: mol_type = other DNA
aaggagcctgcgtccccgttggataaatgccactatactaaggataacggtcagttcgatcagagtgcaaagcaacttaacttggaggcttacactatagagcaagaaacagcgctgataagtaataagaacggtaagccaaagcgacagcagaggaaacccaatcctccgcttaacttggatagctacatcgtcgggcaaaatgacatg

SEQ ID NO: 471 - DNA, 246 bp, synthetic construct
  source 1..246: mol_type = other DNA
ctgaccagaaagaagaaggccctgagaatccacagcgtggaaggcgacctgcggagaaagtctgccggacaagaagagtggtcccctagcgctccatctccacctggatcttgtgtgcaggccgaagcagctcctgctggactgtgtggcgaacagagaggcgaagattgcgccgagctgcacgactacttcaacgtgctgagctacagaagcctgggcaactgcagcttcttcaccgagacagga

SEQ ID NO: 472 - DNA, 210 bp, synthetic construct
  source 1..210: mol_type = other DNA
atggacaaccagggcgtgatctacagcgacctgaacctgcctcctaatcctaagcggcagcagagaaagcccaagggcaacaagaacagcatcctggccaccgagcaagagatcacctacgccgagctgaatctgcagaaggccagccaggacttccagggcaacgacaagacctaccactgcaaggacctgcctagcgctcccgagaag

SEQ ID NO: 473 - DNA, 210 bp, synthetic construct
  source 1..210: mol_type = other DNA
atggacaaccagggcgtcatctacagcgacctgaacctgcctcctaatccaaagcggcagcagcggaagcccaagggcaacaagaatagcatcctggccaccgagcaagagatcacctacgccgagctgaatctgcagaaggccagccaggatttccagggcaacgacaagacctaccactgcaaggacctgcctagcgctcctgagaaa

SEQ ID NO: 474 - DNA, 651 bp, synthetic construct
  source 1..651: mol_type = other DNA
atggaagccattgccaaatacgacttcaaggccaccgccgacgacgagctgagcttcaagagaggcgacatcctgaaggtgctgaacgaggaatgcgaccagaactggtacaaggccgagctgaacggcaaggacggcttcatccccaagaactacatcgagatgaagccccatccatggttcttcggcaagatccccagagccaaggccgaagagatgctgagcaagcagagacacgacggcgcctttctgatccgggaatctgaatctgcccctggcgacttcagcctgagcgtgaagttcggcaacgacgtgcagcacttcaaggtcctgagagatggcgccggaaagtacttcctgtgggtcgtgaagtttaacagcctgaacgagctggtggactaccacagatccaccagcgtgtcccggaaccagcagatcttcctgcgggacatcgaacaggtgccacagcagccaacatacgtgcaggccctgttcgacttcgaccctcaagaggatggcgagctgggctttagacggggcgatttcatccacgtgatggacaacagcgaccccaactggtggaagggcgcttgtcatggacagaccggcatgttccccagaaactacgtgacccctgtgaaccggaacgtg

SEQ ID NO: 475 - DNA, 1443 bp, synthetic construct
  source 1..1443: mol_type = other DNA
atggatggcgccgtgatggaaggccctctgtttctccagagccagagattcggcaccaagcggtggcggaaaacatgggccgttctgtaccctgcctctcctcatggcgtggcccggctggaatttttcgatcacaagggctctagcagcggcggaggcagaggatctagtagacggctggactgcaaagtgatccggctggccgagtgtgtgtctgtggctcctgtgaccgtggaaacccctcctgaacctggcgccacagccttcagactggatacagcccagagaagccatctgctggccgccgatgctccttcttctgctgcttgggtgcagaccctgtgccggaacgcttttcctaaaggcagctggacactggcccctaccgacaatcctcctaagctgagcgccctggaaatgctggaaaacagcctgtacagccccacctgggagggctctcagttttgggtcaccgtgcagagaacagaggccgccgaaagatgtggcctgcacggctcttatgtgctgagagtggaagccgagagactgaccctgctgacagtgggagcccagtctcagatcctggaacctctgctgagctggccctacacactgctgagaagatacggccgggacaaagtgatgttcagcttcgaggccggcagaagatgtccttctggccctggcacattcacatttcagacagcccagggcaacgacatcttccaggctgtggaaaccgccatccacagacagaaggcccagggaaaagccggccagggacacgatgttctgagagccgattctcacgagggcgaagtggccgagggaaagcttccttctccacctggacctcaagagctgctggatagccctcctgctctgtatgccgagcctctggacagcctgagaatcgccccttgtccaagccaggactctctgtacagcgatcccctggatagcacatctgcccaagctggcgaaggcgtgcagaggaagaagcccctgtactgggatctgtacgagcacgctcagcagcaactgctgaaggccaagctgacagaccccaaagaggaccccatctacgacgagcctgaaggacttgctccagtgccacctcagggcctgtacgatctgcctagagagcctaaagacgcctggtggtgtcaggccagagtgaaagaggaaggctacgagctgccttacaaccccgccaccgatgattatgccgtgcctcctccaagaagcaccaaaccactgctggccccaaagcctcagggacctgcttttcctgagcctggaacagccacaggcagcggcatcaagagccacaatagcgccctgtatagccaggtgcagaaaagcggcgccagcggctcttgggattgtggacttagcagagtgggcaccgacaagaccggcgtgaagtctgagggaagcaca

SEQ ID NO: 476 - DNA, 1236 bp, synthetic construct
  source 1..1236: mol_type = other DNA
atgggagatggcgccgtgaagcagggctttctgtatctccagcagcagcagaccttcggcaagaagtggcggagatttggcgcctctctgtacggcggctctgattgtgctctggcccgactggaactgcaagagggacctgagaagcccagaagatgcgaggccgccagaaaagtgatccggctgagcgattgtctgagagtggctgaagcaggcggcgaagccagctctcctagagataccagcgcattcttcctggaaacaaaagagcggctgtacctgctggccgctcctgctgctgaaagaggcgattgggtccaagccatctgcctgctggcttttcccggccagagaaaagagctgtctggccctgagggcaagcagagcaggccttgcatggaagagaacgagctgtactccagcgccgtgacagtgggccctcacaaagaatttgccgtgaccatgaggcccaccgaggccagcgaaagatgtcacctgagaggcagctacaccctgagagccggcgaatctgctctggaactttggggaggacctgagcctggcacacagctgtacgactggccctacagattcctgcggagattcggccgggacaaagtgaccttcagctttgaggctggccgcagatgtgtgtccggcgagggcaatttcgagttcgagacaagacagggcaacgagatcttcctggctctggaagaggccatcagcgcccagaaaaatgctgcccctgctacacctcagcctcagcctgctacaatccctgcctctctgcccagacctgacagcccttatagcagaccccacgactctctgcctccaccttctccaacaacacccgtgcctgctcctagacctagaggacaagagggcgagtacgccgtgccttttgatgccgtggctagaagcctgggcaagaacttcagaggcatcctggctgtgcctccacagctgctggctgaccctctgtacgacagcatcgaggaaaccctgcctccaagacctgaccacatctacgacgagcctgaaggcgtggcagccctgtctctgtatgactcccctcaagagcctagaggcgaagcctggcgtagacaggctaccgccgatagagatcctgccggactgcaacatgtgcagccagccggccaggatttttctgcctctggatggcagccaggcaccgagtacgataacgtggtgctgaagaagggccccaag

SEQ ID NO: 477 - DNA, 804 bp, synthetic construct
  source 1..804: mol_type = other DNA
ccagccccagcggagcgaccgctgccaaaccctgaagggctcgacagtgactttttggctgtcctctccgactatcctagtcccgatatcagccccccgatattcaggcgcggtgaaaaactccgagtcatcagcgatgaggggggttggtggaaagccatcagcctgagtaccggacgagagtcatacattcctggaatatgtgtagcgagggtgtaccacggttggctgttcgagggtctgggaagagataaggccgaggaacttctccaactcccagatacaaaagtcggtagttttatgattcgggaaagtgaaactaaaaaggggttctatagcctctcagttcggcataggcaggtcaagcattatcggatatttcgcttgcctaacaactggtactacataagtccccgactcacattccaatgcctggaggacctcgtgaatcactattcagaagttgcagatggcctctgctgcgtactcacgacaccctgccttacccagagtacagcggccccggctgttcgggcatcttccagcccagtaacactcaggcaaaaaactgtggattggcgcagagtctcacgccttcaggaagatcctgagggtacggaaaatccgctcggtgtggacgagtcactgttctcctatggcttgagggaatctatagcgtcttacctttccttgacgtctgaagataatacgtctttcgatcgcaaaaaaaaatcaatatctcttatgtatggcggaagcaaaaggaaatcatcattcttttcctctccaccatatttcgaagat

SEQ ID NO: 478 - DNA, 717 bp, synthetic construct
  source 1..717: mol_type = other DNA
ccagccccagcggagcgaccgctgccaaaccctgaagggctcgacagtgactttttggctgtcctctccgactatcctagtcccgatatcagccccccgatattcaggcgcggtgaaaaactccgagtcatcagcgatgaggggggttggtggaaagccatcagcctgagtaccggacgagagtcatacattcctggaatatgtgtagcgagggtgtaccacggttggctgttcgagggtctgggaagagataaggccgaggaacttctccaactcccagatacaaaagtcggtagttttatgattcgggaaagtgaaactaaaaaggggttctatagcctctcagttcggcataggcaggtcaagcattatcggatatttcgcttgcctaacaactggtactacataagtccccgactcacattccaatgcctggaggacctcgtgaatcactattcagaagttgcagatggcctctgctgcgtactcacgacaccctgccttacccagagtacagcggccccggctgttcgggcatcttccagcccagtaacactcaggcaaaaaactgtggattggcgcagagtctcacgccttcaggaagatcctgagggtacggaaaatccgctcggtgtggacgagtcactgttctcctatggcttgagggaatctatagcgtcttacctttccttgacgtctgaagataatacgtctttc

SEQ ID NO: 479 - DNA, 762 bp, synthetic construct
  source 1..762: mol_type = other DNA
agaaaatccctccccagtccaagcctgtctagtagcgttcagggtcagggcccagttactatggaagcggaacgatccaaggctaccgcagttgctctgggttcattcccggctggagggccagcggaactctccttgcgcctgggtgagccacttaccatcgtttctgaggacggagattggtggacggttctttctgaagtatctgggagagaatacaacataccgtcagttcatgtcgcaaaagtttcacacggttggctttacgagggactcagcagggaaaaagcggaggaattgttgcttttgcccggcaatcctggcggagcatttttgataagagagagtcagacccggagaggcagttattccctgtcagttagactcagcagacctgcgagttgggataggattcggcactacaggattcactgccttgataacggctggctgtatatatctcctcgcctgacattccctagcctccaagcgttggttgatcattactctgaactggcagacgatatctgctgcctgctcaaggaaccgtgcgtcctccagagggcaggcccacttcctggcaaggatattccacttccagtcacggttcaaagaacccccctcaattggaaggagctggatagctctctcctcttttccgaggccgctacaggtgaagaatctctgttgtctgaaggattgagagagagtctctccttctacatctctctgaatgatgaagcagtgtcattggacgacgca

SEQ ID NO: 480 - DNA, 183 bp, synthetic construct
  source 1..183: mol_type = other DNA
aaagtgaacggctgccggaagtacaagctgaacaagaccgagagcacccctgtggtggaagaggacgagatgcagccttacgccagctacaccgagaagaacaaccctctgtacgacaccaccaacaaagtgaaggccagcgaggccctccagagcgaggttgacacagatctgcacaccctg

SEQ ID NO: 481 - DNA, 252 bp, synthetic construct
  source 1..252: mol_type = other DNA
caccggtggtgcagcaacaagaaaaacgccgccgtgatggaccaagagagcgccggaaatagaaccgccaacagcgaggatagcgacgagcaggaccctcaagaagtgacctacacacagctgaaccactgcgtgttcacccagcggaagatcaccagacctagccagcggcctaagacaccacctaccgacatcatcgtgtacaccgagctgcccaacgccgagagcagatccaaggtcgtgtcctgtcct

SEQ ID NO: 482 - DNA, 123 bp, synthetic construct
  source 1..123: mol_type = other DNA
atgaccgacagcgtgatctacagcatgctcgagctgcctacagccacacaggcccagaatgattacggccctcagcagaagtccagctccagcagacctagctgtagctgtcttggctccggc

SEQ ID NO: 483 - DNA, 303 bp, synthetic construct
  source 1..303: mol_type = other DNA
caccggcagaaccagatcaagcagggccctcctagaagcaaggacgaggaacagaagcctcagcagaggcctgatctggccgtggacgtgctggaaagaacagccgataaggccaccgtgaacggcctgcctgagaaggacagagagacagacacatctgccctggccgctggcagctcccaagaagtgacatacgcccagctggaccactgggccctgacacagagaactgccagagctgtgtcccctcagagcaccaaacctatggccgagagcatcacctatgccgccgtggccagacat

SEQ ID NO: 484 - DNA, 348 bp, synthetic construct
  source 1..348: mol_type = other DNA
ctgcggcacagaaggcagggcaagcactggacaagcacccagagaaaggccgattttcagcaccctgctggcgccgttggacctgagcctacagatagaggactgcagtggcggtctagccctgctgccgatgctcaagaggaaaacctgtacgccgccgtgaaggacacccaacctgaagatggcgtggaaatggacaccagagccgccgcatctgaagcccctcaggatgtgacatacgcccagctgcatagcctgacactgaggcggaaagccacagagcctccacctagccaagagagagagcctcctgccgagcctagcatctatgccacactggccattcac

SEQ ID NO: 485 - DNA, 501 bp, synthetic construct
  source 1..501: mol_type = other DNA
cggagacagagacacagcaagcacagaaccagcgaccagagaaagaccgacttccagaggcctgctggcgccgctgagacagagcctaaagatagaggactgctgcggagaagcagccctgctgccgatgtgcaagaggaaaatctgtacgccgccgtgaaggacacccagagcgaggatagagtggaactggacagccagtctcctcacgacgaagatcctcaggccgtgacatacgcccctgtgaagcacagcagccctcggagagaaatggcctctccaccttctagcctgagcggcgagttcctggacaccaaggacagacaggtggaagaggaccggcagatggatacagaagctgccgcctctgaagccagccaggatgtgacatatgcccagctgcatagcctgacactgcggcggaaagctacagagcctcctccatctcaagagggcgagcctccagccgagcctagcatctatgccacactggccattcac

SEQ ID NO: 486 - DNA, 504 bp, synthetic construct
  source 1..504: mol_type = other DNA
cagcattggagacagggaaagcacagaacactggcccagcggcaggccgatttccaaagacctccaggtgccgccgaacctgagcctaaagatggtggcctgcagcggagatcttctcctgctgccgatgtgcagggcgagaatttttgtgccgccgtgaagaacacccagcctgaggatggcgtggaaatggacaccagacagagccctcacgacgaggatccacaggccgtgacatacgccaaagtgaagcacagccggcctcggagagaaatggctagccctccaagtcctctgagcggcgagttcctggacaccaaagacagacaggccgaagaggaccggcagatggatacagaagctgccgcctctgaagcccctcaggatgtgacatatgcccagctgcatagcttcaccctgcggcagaaagccacagagcctccaccttctcaagagggcgcttctccagccgagccatctgtgtatgccacactggccattcac

SEQ ID NO: 487 - DNA, 423 bp, synthetic construct
  source 1..423: mol_type = other DNA
aagctgcagcggagatggaagagaacacagagccagcagggcctgcaagagaatagcagcggccagagcttcttcgtgcggaacaagaaagtgcggagagcccctctgtctgagggacctcatagcctgggctgttacaaccccatgatggaagatggcatcagctacaccacactgcggttccccgagatgaacatccctagaacaggcgacgccgagagcagcgaaatgcaaagacctcctcctgactgcgacgacaccgtgacatatagcgccctgcacaagagacaagtgggcgactacgagaacgtgatccctgacttccctgaggacgagggcatccactacagcgagctgatccagtttggcgtgggcgaaagaccccaggctcaagagaatgtggactacgtgatcctgaagcac

SEQ ID NO: 488 - DNA, 378 bp, synthetic construct
  source 1..378: mol_type = other DNA
aagatcctgcctaagcggcgcacccagaccgagactcctagacctagattcagccggcacagcaccatcctggactacatcaacgtggtgcctaccgccggaccactggctcagaagagaaaccagaaggccacacctaacagccccagaacacctcttccacctggcgcaccttctccagagagcaagaagaaccagaagaagcagtaccagctgcctagcttccccgagcctaagagcagtacacaggcccctgagagccaagagtcccaagaggaactgcactacgccacactgaacttccccggcgtcagacctagacctgaggccagaatgcctaagggcacccaggccgattacgccgaagtgaagtttcaa

SEQ ID NO: 489 - protein, 512 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 490 - protein, 460 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 491 - protein, 511 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDVLIGVSVAFVLLLFLLLFLLLRRQRHSKHRTSDQRKTDFQRPAGAAETEPKDRGLLRRSSPAADVQEENLYAAVKDTQSEDRVELDSQSPHDEDPQAVTYAPVKHSSPRREMASPPSSLSGEFLDTKDRQVEEDRQMDTEAAASEASQDVTYAQLHSLTLRRKATEPPPSQEGEPPAEPSIYATLAIH

SEQ ID NO: 492 - protein, 512 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDVLIGVLVVSILLLSLLLFLLLQHWRQGKHRTLAQRQADFQRPPGAAEPEPKDGGLQRRSSPAADVQGENFCAAVKNTQPEDGVEMDTRQSPHDEDPQAVTYAKVKHSRPRREMASPPSPLSGEFLDTKDRQAEEDRQMDTEAAASEAPQDVTYAQLHSFTLRQKATEPPPSQEGASPAEPSVYATLAIH

SEQ ID NO: 493 - protein, 426 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDILIGTSVVIILFILLFFLLHRWCSNKKNAAVMDQESAGNRTANSEDSDEQDPQEVTYTQLNHCVFTQRKITRPSQRPKTPPTDIIVYTELPNAESRSKVVSCP

SEQ ID NO: 494 - protein, 427 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDILIGTSVVIILFILLLFFLLHLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 495 - protein, 596 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIHHLWCSNKKNAAVMDQEPAGNRTANSEDSDEQDPEEVTYAQLDHCVFTQRKITRPSQRPKTPPTDTILYTELPNAKPRSKVVSCP

SEQ ID NO: 496 - protein, 427 aa, synthetic construct; identical to SEQ ID NO: 494

SEQ ID NO: 497 - protein, 512 aa, synthetic construct; identical to SEQ ID NO: 489

SEQ ID NO: 498 - protein, 455 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 499 - protein, 483 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 500 - protein, 470 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDGAFLGIGITALLFLCLALIIMKILPKRRTQTETPRPRFSRHSTILDYINVVPTAGPLAQKRNQKATPNSPRTPLPPGAPSPESKKNQKKQYQLPSFPEPKSSTQAPESQESQEELHYATLNFPGVRPRPEARMPKGTQADYAEVKFQ

SEQ ID NO: 501 - protein, 385 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDVAIALGLLTAVLLSVLLYQWIMTDSVIYSMLELPTATQAQNDYGPQQKSSSSRPSCSCLGSG

SEQ ID NO: 502 - protein, 445 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDILIGVSVVFLFCLLLLVLFCLHRQNQIKQGPPRSKDEEQKPQQRPDLAVDVLERTADKATVNGLPEKDRETDTSALAAGSSQEVTYAQLDHWALTQRTARAVSPQSTKPMAESITYAAVARH

SEQ ID NO: 503 - protein, 414 aa, synthetic construct
MALPVTALLLPLALLLHAARPQVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSSGGGGSGGGGSGGGGSDIVMTQTPPSLSVALGQSVSISCKSSQSLVASDENTYLNWLLQSPGRSPKRLIYQVSKLDSGVPDRFSGSGSEKDFTLKISRVEAEDLGVYYCLQGIHLPWTFGGGTKLELKGKPIPNPLLGLDSTNGAATTTPAPRPPTPAPTIALQPLSLRPEACRPAAGGAVHTRGLDFACDVIGILVAVILLLLLLLLLFLIMDNQGVIYSDLNLPPNPKRQQRKPKGNKNSILATEQEITYAELNLQKASQDFQGNDKTYHCKDLPSAPEK

SEQ ID NO: 504 - protein, 10 aa, synthetic construct
GSGSGSGSGG

SEQ ID NO: 505 - protein, 17 aa, synthetic construct
AEAAAKEAAAKEAAAKA

SEQ ID NO: 506 - protein, 4 aa, synthetic construct
KEGS

SEQ ID NO: 507 - protein, 8 aa, synthetic construct
EPLFAERK

SEQ ID NO: 508 - protein, 5 aa, synthetic construct
  VARIANT 2: note = X can be G or A
GXELR

SEQ ID NO: 509 - protein, 6 aa, synthetic construct
  VARIANT 4..5: note = X can be any amino acid
DSGXXS

SEQ ID NO: 510 - DNA, 366 bp, synthetic construct
  source 1..366: mol_type = other DNA
gaagttcagctggttcagagcggagccgaagtgaagaaacctggcagcagcgtgaaggtgtcctgcaaagcttctggcggcaccttcagcagctacgccatctcttgggttcgacaggcacctggacagggcctcgagtggatgggaggaatcatccctatcttcggcaccgccaattacgcccagaaatttcagggaagagtgaccatcaccgccgacaagagcaccagcaccgcctacatggaactgagcagcctgagaagcgaggataccgctgtgtattactgcgccacattcgccctgttcggcttcagagagcaggccttcgatatctggggccagggcacaaccgtgacagtttcatct

SEQ ID NO: 511 - DNA, 348 bp, synthetic construct
  source 1..348: mol_type = other DNA
caggttcagcttgtgcaatctggcgctgaagtgaaaaagcccggctcctccgtgaaagtgtcttgtaaagccagcggctacacctttaccgactacaatatgcattgggtccgccaggcaccaggccagggacttgaatggatcggctacatctacccctacaacggcggcaccggctacaaccagaagttcaagtccaaggccacaatcacagccgacgagtccaccaatactgcttatatggaactgtctagccttcggagcgaggacacagccgtgtactattgcgctagaggcagacccgccatggactattggggacagggaaccctggtcacagtgtccagc

SEQ ID NO: 512 - DNA, 333 bp, synthetic construct
  source 1..333: mol_type = other DNA
gacatccagatgacacagagccctagtagcctgagcgccagcgtgggagacagagtgacaattacctgtcgggccagcgagagcgtggacaactacggcatcagcttcatgaactggttccagcagaagcctggcaaggcccctaagctgctgatctatgccgccagcaatcaaggcagcggagtgccctcaagattcagcggaagcggctccggcaccgactttaccctgacaatcagctccctgcagcctgacgacttcgccacctactactgccagcagagcaaagaggtgccctggacattcggacagggcaccaaggtggaaatcaaa

SEQ ID NO: 513 - DNA, 321 bp, synthetic construct
  source 1..321: mol_type = other DNA
gatattcagatgactcagtccccaagcagcctgtcagcctccgtgggcgatagagtcaccattacatgcagagccagccagtccatctccagctacctgaactggtatcagcaaaaacccggaaaggctccaaagctcctcatctacgccgcttctagcctgcagtctggcgtgccatctagattctctggctccggaagcgggaccgacttcacactgaccatatcttctctgcagcccgaagatctggccacgtactattgtcagcagtcctacagcacccctttcaccttcggacccggaacaaaggtggacattaag

SEQ ID NO: 514 - DNA, 336 bp, synthetic construct
  source 1..336: mol_type = other DNA
gaggtgcagctggttgaatctggcggaggactggttcagcctggcggatctctgagactgtcttgtgccgccagcggcttcaccttcagcagatacgatatgcactgggtccgacaggcccctggcaaaggacttgaatgggtgtccgtgatctggggcaacggcaacacacactaccacagcgccctgaagtcccggttcaccatctccagagacaacagcaagaacaccctgtacctgcagatgaacagcctgagagccgaggacaccgccgtgtactactgcaccctgagaatcaaggattggggccagggcaccatggtcaccgtttcttct

SEQ ID NO: 515 - DNA, 336 bp, synthetic construct
  source 1..336: mol_type = other DNA
gacgtggtcatgacacagagccctctgagcctgcctgtgacactgggacagcctgccagcatcagctgcaagtctagccagtctctggtggccagcgacgagaacacctacctgaactggttccagcagaggcccggacagtctcctagacggctgatctaccaggtgtccaagctggatagcggcgtgcccgatagattttctggcagcggctctggcaccgacttcaccctgaagatcagcagagtggaagccgaggacgtgggcgtgtactactgtctgcaaggcatccatctgccttggacctttggccagggcacaaagctggaaatcaag

SEQ ID NO: 516 - DNA, 16 bp, synthetic construct
  source 1..16: mol_type = other DNA
ggcggcggtggctctc

SEQ ID NO: 517 - DNA, 16 bp, synthetic construct
  source 1..16: mol_type = other DNA
ggtggcggcggatccg

SEQ ID NO: 518 - DNA, 336 bp, synthetic construct
  source 1..336: mol_type = other DNA
agagtgaagtttagcagaagcgccgacgctcccgcctataagcagggccagaatcagctgtacaacgagctgaatctggggcgaagagaagagtacgacgtgctggacaaaagaagaggcagggaccctgagatgggcggcaagcccagaagaaagaaccctcaagagggcctgtataatgagctgcagaaagacaagatggccgaggcctacagcgagatcggaatgaagggcgagcgtcggagaggcaagggacacgatggactgtatcagggcctgagcaccgccaccaaggatacctacgatgccctgcacatgcaggccctgcctccaaga

SEQ ID NO: 519 - DNA, 123 bp, synthetic construct
  source 1..123: mol_type = other DNA
cagtgcacaaactacgcacttttaaaactcgcaggggatgtggagtctaatcctggccccggaagcggagaaggtcgtggtagtctactaacgtgtggtgatgtagaagaaaatcctggacct

SEQ ID NO: 520 - DNA, 336 bp, synthetic construct
  source 1..336: mol_type = other DNA
gatatcgtgatgacccagacacctcctagcctgtctgtggctctgggccagtctgtgtccatcagctgcaagagcagccagagcctggtggcctccgacgagaacacctacctgaattggctgctgcagagccccggcagaagccccaagagactgatctaccaggtgtccaagctggacagcggcgtgcccgatagattttctggcagcggcagcgagaaggacttcaccctgaagatctccagagtggaagccgaggacctgggcgtgtactactgtctgcaaggcatccatctgccttggacctttggaggcggcacaaagctggaactgaaa

SEQ ID NO: 521 - DNA, 135 bp, synthetic construct
  source 1..135: mol_type = other DNA
accacgacgccagcgccgcgaccaccaacaccggcgcccaccatcgcgttgcagcccctgtccctgcgcccagaggcgtgccggccagcggcggggggcgcagtgcacacgagggggctggacttcgcctgtgat

SEQ ID NO: 522 - DNA, 63 bp, synthetic construct
  source 1..63: mol_type = other DNA
gttatagggatcctggtggctgtcatactcctcttgctcctcttgttgctgctttttttgata

SEQ ID NO: 523 - DNA, 336 bp, synthetic construct
  source 1..336: mol_type = other DNA
caggtgcagctgaaagagtctggacctggactggtgcagcccagccaaacactgagcctgacctgtaccgtgtccggcttcagcctgagcagatacgacatgcactgggtccgacagcctccaggacaaggcttggaatggatgggcgtgatctggggcaacggcaacacacactatcacagcgccctgaagtcccggctgagcatcagcagagataccagcaagagccaggtgttcctgaagatgaacagcctgcagaccgaggacaccgccatctatttctgcaccctgcggatcaaggattggggccctggcacaatggtcaccgtttctagc

SEQ ID NO: 524 - protein, 112 aa, synthetic construct
QVQLKESGPGLVQPSQTLSLTCTVSGFSLSRYDMHWVRQPPGQGLEWMGVIWGNGNTHYHSALKSRLSISRDTSKSQVFLKMNSLQTEDTAIYFCTLRIKDWGPGTMVTVSS

SEQ ID NO: 525 - DNA, 336 bp, synthetic construct
  source 1..336: mol_type = other DNA
caggtccagctgaaagaaagcggccctggactggtgcagcctagccaaacactgagcctgacctgtaccgtgtccggctttagcctgtccagatacgatatgcattgggtccgccagccaccaggacagggactcgaatggatgggagtgatctggggcaacggcaacacccactaccacagcgccctgaagtccagactgagcatctccagagacaccagcaagtcccaggtgttcctgaagatgaacagcctgcagaccgaagataccgccatctatttctgcaccctgcggatcaaggattggggcccaggcacaatggtcacagtttcttcc

SEQ ID NO: 526 - DNA, 45 bp, synthetic construct
  source 1..45: mol_type = other DNA
ggcggaggcggttctggcggaggcggtagcggcggaggcggctct

SEQ ID NO: 527 - DNA, 336 bp, synthetic construct
  source 1..336: mol_type = other DNA
gatattgtgatgacacaaacccctccaagcctgagcgtggcactgggacagtctgtgtccatcagctgcaagagcagccagtcactggtggccagcgacgagaacacatacctgaattggctgctgcagagcccaggcagaagcccaaaacggctgatctatcaggtgtccaagctggacagcggagtgcccgatagattcagcggatccggctctgagaaggactttaccctgaagatcagtcgcgtggaagccgaagatctgggcgtctactactgtctgcaaggcatccatctgccttggacctttggcggagggaccaaactggaactgaag